(19) United States
(12) Patent Application Publication
(10) Pub. No.: US 2017/0281641 A1
(43) Pub. Date: Oct. 5, 2017

(54) CYCLIC UREA COMPOUNDS AS TROPOMYOSIN-RELATED KINASE (TRK) iNHIBITORS

(71) Applicant: GENZYME CORPORATION, Cambridge, MA (US)

(72) Inventors: Kerry DONAHUE, Littleton, MA (US); C. Michael PHILBROOK, Boston, MA (US); Dominick BLASIOLI, North Chelmsford, MA (US); John L. KANE, Maynard, MA (US); Carl FLANNERY, Acton, MA (US)

(21) Appl. No.: 15/508,738

(22) PCT Filed: Sep. 2, 2015
(86) PCT No.: PCT/US2015/048047

§ 371 Date: Mar. 3, 2017

Related U.S. Application Data
(60) Provisional application No. 62/045,231, filed on Sep. 3, 2014.

(51) Int. Cl. A61K 31/55; A61K 31/4439; A61K 31/5377; A61K 31/496; A61K 31/444; A61K 31/4545; A61K 31/497; A61K 31/4245; A61K 31/4178; A61K 31/422; A61K 31/454; A61K 31/427; A61K 31/506; A61K 31/517; A61K 31/4709

(52) U.S. Cl. CPC A61K 31/55; A61K 31/4709; A61K 31/4439; A61K 31/5377; A61K 31/496; A61K 31/444; A61K 31/4545; A61K 31/497; A61K 31/4245; A61K 31/4178; A61K 31/422; A61K 31/454; A61K 31/427; A61K 31/506; A61K 31/517

(57) ABSTRACT

Tropomyosin-related kinase inhibitors (Trk inhibitors) are small molecule compounds useful in the treatment of disease. Trk inhibitors can be used as pharmaceutical agents and in pharmaceutical compositions. Trk inhibitors are useful in the treatment of inflammatory diseases, autoimmune disease, defects of bone metabolism and/or cancer, and are particularly useful in the treatment of osteoarthritis (OA), pain, and pain associated with OA. Trk inhibitors are also useful for inhibiting tropomyosin-related kinase A (TrkA), tropomyosin-receptor kinase B (TrkB), tropomyosin-receptor kinase C (TrkC), and/or c-FMS (the cellular receptor for colony stimulating factor-1 (CSF-1)).

### CROSS-REFERENCE TO RELATED APPLICATIONS

This application claims the benefit of priority to U.S. Provisional Application No. 62/045,231 filed Sep. 3, 2014, the disclosure of which is incorporated herein in its entirety.

### BACKGROUND OF THE INVENTION

### Field of the Invention

This invention relates to cyclic urea compounds as tropomyosin-related kinase inhibitors. This invention also relates to pharmaceutical compositions comprising cyclic urea compounds and to the use of cyclic urea compounds and pharmaceutical compositions comprising cyclic urea compounds to treat disease. The cyclic urea compounds of the invention function by inhibiting the activity of a protein kinase, specifically tropomyosin-related kinase (Trk). This invention further relates to the use of cyclic urea compounds to treat inflammatory diseases and/or defects of bone metabolism. The cyclic urea compounds of the present invention can be used to treat osteoarthritis (OA), to treat pain, to treat pain associated with OA, and to inhibit tropomyosin-related kinases including tropomyosin-related kinase A (TrkA), tropomyosin-related kinase B (TrkB), and tropomyosin-related kinase C (TrkC).

### BRIEF SUMMARY OF THE INVENTION

The present invention relates to cyclic urea compounds of Formula (I):

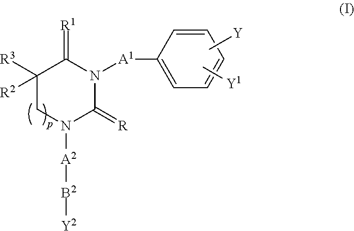

wherein:
p is 0, 1 or 2;
R and R¹, which may be identical or different, are O or NH;
R² and R³, which may be identical or different, are hydrogen, alkyl, alkenyl, alkynyl, cycloalkyl, aryl and heteroaryl that are optionally substituted, or R² and R³ taken together with the carbon atom to which they are attached form 3- to 10-membered carbocyclyl that is optionally being substituted or 3- to 10-membered heterocyclyl containing one or more hetero atoms chosen from O, S, N and NR⁷ that is optionally being substituted;
A¹ is single bond, alkyl, alkenyl or alkynyl;
Y and Y¹, which may be identical or different, are such that one of Y and Y¹ is —OCF₃, —O—F₂—CHF₂, —O—CHF₂, —O—CH₂—CF₃, —SO₂NR⁵R⁶, —SF₅ and —S(O)ₙ-alkyl and the other of Y and Y¹ is —OCF₃, —O—F₂—CHF₂, —O—CHF₂, —O—CH₂—CF₃, SO₂NR⁵R⁶, —SF₅, —S(O)ₙ-alkyl, hydrogen, halogen, hydroxyl, alkoxy, nitro, —CN, —NR⁵R⁶, optionally substituted alkyl, optionally substituted aryl, optionally substituted heteroaryl, —CF₃, —O-alkenyl, —O-alkynyl, —O-cycloalkyl, —S(O)ₙ-alkenyl, —S(O)ₙ-alkynyl, —S(O)ₙ-cycloalkyl, —CONR⁵R⁶, or free, salified or esterified carboxyl, or the

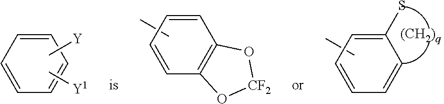

that is optionally substituted with one or more alkyl that are optionally substituted, wherein
   q is 2, 3 or 4;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
A², which may be identical to or different from A¹, is defined as A¹ or is CO or SO₂,
B² is saturated or unsaturated, 3- to 10-membered monocyclic or bicyclic heterocyclyl containing one or more hetero atoms chosen from O, S, N and NR⁷ that is optionally substituted with one or more identical or different substituents defined as Y², wherein
   R⁷ is hydrogen, alkyl, cycloalkyl, phenyl, acyl, —S(O)₂Alk, —S(O)₂Aryl, —S(O)₂heteroaryl or —S(O)₂NR⁵R⁶,
   Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹⁰—CS—NR⁵R⁶ or —NR¹⁰—COOR¹³, all of which are optionally substituted;
all the alkyl, alkenyl, alkynyl and alkoxy above are linear or branched and contain not more than 6 carbon atoms,
all the cycloalkyl and heterocyclyl above contain not more than 7 carbon atoms,
all the aryl and heteroaryl above contain not more than 10 carbon atoms,
all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²,
all the aryl and heteroaryl above are optionally substituted with one or more substituents selected from the group consisting of alkyl, alkoxy and alkylenedioxy,
all the cyclic radicals above, and also the ring formed by R⁵ and R⁶ with the nitrogen atom to which they are attached, are optionally substituted with one or more substituents selected from the group consisting of oxo and thioxo,
n is 0 to 2, and wherein
   R⁸ is alkyl, alkenyl, cycloalkyl, cycloalkylalkyl, heterocyclyl, heterocyclylalkyl, aryl, arylalkyl, heteroaryl and heteroarylalkyl,
   R⁹ is defined as R⁸ or is hydrogen,
   R¹⁰ is hydrogen or alkyl,
   R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl, or R¹¹ and R¹² taken together with the nitrogen atom to which they are attached form 5- to 7-membered cyclic radical containing one or more hetero atoms chosen from O, S, N and NR⁷, preferably a cyclic amine, and
   R¹³, which may be identical to or different to R⁵ or R⁶, is defined as R⁵ or R⁶, or
      a racemic, enantiomeric or diastereoisomeric isomer form of the compound of Formula (I), addition salt with mineral or organic acid or with mineral or organic base thereof, with the proviso:
a) when p is 0, R and R¹ are oxygen, A¹ is single bond or alkyl, Y and Y¹, which may be identical or different, are at least one is —OCF₃ or —S-alk, A² is single bond or alkyl and B² is an optionally substituted heterocyclyl, then R² and R³ are not one hydrogen and the other imidazolylalkyl;
b) when p is 0, R and R¹ are oxygen, A¹ is single bond or alkyl, Y and Y¹, which may be identical or different, are at least one is —OCF₃, —SO-Alk, —S(O)₂-alk or —SO₂NH₂, A² is CH₂ and B² is an optionally substituted heterocyclyl, then R² and R³ are not one hydrogen and the other alkyl optionally interrupted with O, S or N-alk; always substituted with a hydroxamate (—CO—NHOH);
c) when p is 0, R and R¹ are oxygen, A¹ is a single bond or alkyl, Y and Y¹, which may be identical or different, are at least one is —S(O)ₙ-alk, A² is single bond and B² is an optionally substituted 5- or 6-membered aromatic heterocyclyl, then R² and R³ are not selected from the group consisting of hydrogen, alkyl, arylalkyl, aryl and heteroaryl; or
d) when p is 0 to 2, R and R¹ are oxygen, A¹ is single bond, Y and Y¹, which may be identical or different, are one is —SO₂Alk or SO₂NH₂ and the other is NR⁵R⁶, A² is single bond or alkylene and B² is optionally substituted 5- to 10-membered heterocyclyl, then R² and R³ are not both hydrogen.

The present invention further relates to methods of treating inflammatory diseases and/or defects of bone metabolism in a patient in need thereof comprising administering to the patient a compound according to Formula (I).

The present invention further relates to methods of treating osteoarthritis in a patient in need thereof comprising administering to the patient a compound according to Formula (I).

The present invention further relates to methods of treating pain in a patient in need thereof comprising administering to the patient a compound according to Formula (I).

The present invention further relates to methods of treating pain associated with osteoarthritis in a patient in need thereof comprising administering to the patient a compound according to Formula (I).

The present invention relates to methods of inhibiting tropomyosin-receptor kinase in a patient comprising administering to the patient a compound according to Formula (I). The invention further relates to methods of inhibiting tropomyosin-receptor kinase A, methods of inhibiting tropomyosin-receptor kinase B, and methods of inhibiting tropomyosin-receptor kinase C in a patient comprising administering to the patient a compound according to Formula (I).

### DETAILED DESCRIPTION OF THE INVENTION

This invention relates to tropomyosin-related kinase inhibitors (Trk inhibitors). This invention also relates to pharmaceutical compositions comprising Trk inhibitors and to the use of Trk inhibitors and pharmaceutical compositions comprising Trk inhibitors to treat disease. This invention further relates to the use of Trk inhibitors to treat inflammatory diseases, autoimmune disease, defects of bone metabolism and cancer. The Trk inhibitors of the present invention can be used to treat osteoarthritis (OA), to treat pain associated with OA, and to inhibit tropomyosin-related kinase A (TrkA), tropomyosin-receptor kinase B (TrkB), tropomyosin-receptor kinase C (TrkC), and to inhibit c-FMS (the cellular receptor for colony stimulating factor-1 (CSF-1)).

Tropomyosin-related kinases (Trk) are high affinity receptors activated by soluble growth factors called neutrophins (NT). TrkA, also known as neurotrophic tyrosine kinase receptor type 1, is activated by nerve growth factor (NGF). TrkB is activated by brain derived growth factor and NT-4/5. TrkC is activated by NT3. The activation of Trk leads to the activation of downstream kinases that are implicated in cell signaling, including cell proliferation, survival, angiogenesis and metastasis. Trk have been implicated in a number of diseases, including OA.

OA is a prevalent and debilitating joint disease characterized by chronic pain and destruction of articular cartilage. Recent clinical trials have confirmed a role for blocking NGF in OA knee pain, demonstrating significant pain relief and high responder rates in patients treated by intravenous infusion with anti-NGF blocking antibodies (Lane, 2010, N Engl J Med). However, this modality may lead to an increased risk for adverse events due to systemic inhibition of NGF signaling (FDA Arthritis Advisory Committee Meeting to Discuss Safety Issues Related to the Anti-Nerve Growth Factor Agents; http://www.fda.gov/AdvisorvCommittees/Calendar/ucm286556.htm) Accordingly, a novel approach toward targeting NGF-mediated OA pain has been adopted through the development of Trk inhibitors, specifically TrkA inhibitors, the high-affinity receptor for NGF (Nicol, 2007, Molecular Interv). The Trk inhibitors of the present invention are delivered locally and thereby avoid the systemic distribution observed with intravenous anti-NGF administration. This treatment strategy provides enhanced dosing convenience, as well greater safety by allowing for the maintenance of physiologically necessary NGF signaling (i.e. sensory/sympathetic nerve maintenance, angiogenesis) at non-local sites.

As used above, and throughout the description of the invention, the following terms, unless otherwise indicated, shall be understood to have the following meanings:

The term “Hal”, “Halo” or halogen denotes fluorine, chlorine, bromine or iodine atoms.

The term “alkyl”, “alk”, “Alk” or “ALK” denotes a linear or branched radical containing not more than 12 carbon atoms, chosen from methyl, ethyl, propyl, isopropyl, butyl, isobutyl, sec-butyl, tert-butyl, pentyl, isopentyl, sec-pentyl, tert-pentyl, neopentyl, hexyl, isohexyl, sec-hexyl, tert-hexyl, heptyl, octyl, nonyl, decyl, undecyl and dodecyl radicals, and also the linear or branched positional isomers thereof.

Mention is made more particularly of alkyl radical containing not more than 6 carbon atoms, and especially methyl, ethyl, propyl, isopropyl, n-butyl, isobutyl, tert-butyl, linear or branched pentyl and linear or branched hexyl.

The term “alkenyl” denotes a linear or branched radical containing not more than 12 carbon atoms and preferably 4 carbon atoms, chosen, for example, from the following values: ethenyl or vinyl, propenyl or allyl, 1-propenyl, n-butenyl, i-butenyl, 3-methyl-2-butenyl, n-pentenyl, hexenyl, heptenyl, octenyl, cyclohexylbutenyl and decenyl, and also the linear or branched positional isomers thereof, more particularly is allyl or butenyl.

The term “alkynyl” denotes a linear or branched radical containing not more than 12 carbon atoms and preferably 4 carbon atoms, chosen, for example, from the following values: ethynyl, propynyl or propargyl, butynyl, n-butynyl, i-butynyl, 3-methyl-2-butynyl, pentynyl or hexynyl, and also the linear or branched positional isomers thereof; more particularly is propargyl.

The term “alkoxy” denotes a linear or branched radical containing not more than 12 carbon atoms and preferably 6 carbon atoms chosen, for example, from methoxy, ethoxy, propoxy, isopropoxy, linear, secondary or tertiary butoxy, pentoxy, hexoxy and heptoxy radicals, and also the linear or branched positional isomers thereof.

The term “alkoxycarbonyl” or alkyl-O—CO— denotes a linear or branched radical containing not more than 12 carbon atoms, in which the alkyl radical has the meaning given above: examples include methoxycarbonyl and ethoxycarbonyl radicals.

The term “alkylenedioxy” or —O-alkylene-O— denotes a linear or branched radical containing not more than 12 carbon atoms, in which the alkylene radical has the meaning given above: examples include methylenedioxy and ethylenedioxy radicals.

The term “alkylsulfinyl” or alkyl-SO— denotes a linear or branched radical containing not more than 12 carbon atoms, in which the alkyl radical has the meaning given above and preferably contains 4 carbon atoms.

The term “alkylsulfonyl” or alkyl-SO₂— denotes a linear or branched radical containing not more than 12 carbon atoms, in which the alkyl radical has the meaning given above and preferably contains 4 carbon atoms.

The term “alkylsulfonylcarbamoyl” or alkyl-SO₂—NH—C(═O)— denotes a linear or branched radical containing not more than 12 carbon atoms, in which the alkyl radical has the meaning given above and preferably contains 4 carbon atoms.

The term “alkylthio” or alkyl-S— denotes a linear or branched radical containing not more than 12 carbon atoms and especially is methylthio, ethylthio, isopropylthio and heptylthio radicals.

The term “cycloalkyl” denotes a 3- to 10-membered monocyclic or bicyclic carbocyclic or carbocyclyl radical and especially denotes cyclopropyl, cyclobutyl, cyclopentyl and cyclohexyl radicals.

The term “—O-cycloalkyl” denotes a radical in which the cycloalkyl radical has the meaning given above.

The term “cycloalkenyl” denotes a 3- to 10-membered monocyclic or bicyclic nonaromatic carbocyclic or carbocyclyl radical containing at least one double bond, and especially denotes cyclobutenyl, cyclopentenyl and cyclohexenyl radicals.

The term “cycloalkylalkyl” denotes a radical in which cycloalkyl and alkyl are chosen from the values indicated above: this radical thus denotes, for example, cyclopropylmethyl, cyclopentylmethyl, cyclohexylmethyl and cycloheptylmethyl radicals.

The term “acyl” or rad-CO— denotes a linear or branched radical containing not more than 12 carbon atoms, in which the rad is hydrogen, alkyl, cycloalkyl, cycloalkenyl, cycloalkyl, heterocyclyl or aryl radical, these radicals having the values indicated above and being optionally substituted as indicated: examples include the formyl, acetyl, propionyl, butyryl or benzoyl radicals, valeryl, hexanoyl, acryloyl, crotonoyl or carbamoyl.

The term “acyloxy” means acyl-O— radical in which acyl has the meaning given above: examples include acetoxy or propionyloxy radicals.

The term “acylamino” means acyl-NH— radical in which acyl has the meaning given above.

The term “aryl” denotes unsaturated monocyclic radical or unsaturated radical consisting of fused carbocyclic rings. Examples of such aryl radicals that may be mentioned include phenyl or naphthyl radicals. Mention is made more particularly of the phenyl radical.

The term “arylalkyl” means radical resulting from the combination of the optionally substituted alkyl radicals mentioned above and the optionally substituted aryl radicals also mentioned above: examples include benzyl, phenylethyl, 2-phenethyl, triphenylmethyl or naphthalenemethyl radicals.

The term “heterocyclic” denotes a saturated carbocyclic radical (heterocyclyl) or unsaturated carbocyclic radical (heteroaryl) which is at least 6-membered, interrupted with one or more hetero atoms, which may be identical or different, chosen from oxygen, nitrogen and sulfur atoms.

Heterocyclyl radical that may especially be mentioned include dioxolane, dioxane, dithiolane, thiooxolane, thiooxane, oxiranyl, oxolanyl, dioxolanyl, piperazinyl, piperidyl, pyrrolidyl, imidazolidinyl, pyrazolidinyl, morpholinyl, or tetrahydrofuryl, tetrahydrothienyl, chromanyl, dihydrobenzofuranyl, indolinyl, piperidyl, perhydropyranyl, pyrindolinyl, tetrahydroquinolyl, tetrahydroisoquinolyl and thioazolidinyl radicals, all these radicals being optionally substituted.

Among the heterocyclyl radical that may especially be mentioned are optionally substituted piperazinyl, optionally substituted piperidyl, optionally substituted pyrrolidinyl, imidazolidinyl, pyrazolidinyl, morpholinyl and thioazolidinyl radicals: mention may also be made more particularly of optionally substituted morpholinyl, pyrrolidyl and piperazinyl radicals.

The term “heterocyclylalkyl” means radical in which the heterocyclyl and alkyl residues have the above meanings.

Among the 5-membered heteroaryl radicals that may be mentioned are furyl radicals such as 2-furyl, thienyl radicals such as 2-thienyl and 3-thienyl, and pyrrolyl, diazolyl, thiazolyl, thiadiazolyl, thiatriazolyl, isothiazolyl, oxazolyl, oxadiazolyl, 3- or 4-isoxazolyl, imidazolyl, pyrazolyl and isoxazolyl radicals.

Among the 6-membered heteroaryl radicals that may especially be mentioned are pyridyl radicals such as 2-pyridyl, 3-pyridyl and 4-pyridyl, and pyrimidyl, pyrimidinyl, pyridazinyl, pyrazinyl and tetrazolyl radicals.

Among the fused heteroaryl radicals containing at least one hetero atom chosen from sulfur, nitrogen and oxygen, examples include benzothienyl such as 3-benzothienyl, benzofuryl, benzofuranyl, benzopyrrolyl, benzimidazolyl, benzoxazolyl, thionaphthyl, indolyl, purinyl, quinolyl, isoquinolyl and naphthyridinyl.

Among the fused heteroaryl radicals that may be mentioned more particularly are benzothienyl, benzofuranyl, indolyl, quinolyl, benzimidazolyl, benzothiazolyl, furyl, imidazolyl, indolizinyl, isoxazolyl, isoquinolyl, isothiazolyl, oxadiazolyl, pyrazinyl, pyridazinyl, pyrazolyl, pyridyl, pyrimidinyl, pyrrolyl, quinazolinyl, 1.3.4-thiadiazolyl, thiazolyl and thienyl radicals and triazolyl groups, these radicals optionally being substituted as indicated for the heteroaryl radicals;

The term “cyclic amine” denotes a 3- to 8-membered cycloalkyl radical in which one carbon atom is replaced with a nitrogen atom, the cycloalkyl radical having the meaning given above and also possibly containing one or more other hetero atoms chosen from O, S, SO₂, N and NR7 with R7 as defined above; examples of such cyclic amines that may be mentioned include pyrrolidyl, piperidyl, morpholinyl, piperazinyl, indolinyl, pyrindolinyl and tetrahydroquinolyl radicals.

The term “patient” denotes a human being or other mammal.

The term “prodrug” denotes a compound that may be converted in vivo via metabolic mechanisms (such as hydrolysis) into a product of Formula (I). For example, an ester of a compound of Formula (I) containing a hydroxyl group may be converted by hydrolysis in vivo into its parent molecule. Alternatively, an ester of a compound of Formula (I) containing a carboxyl group may be converted by in vivo hydrolysis into its parent molecule.

Examples of esters of the compound of Formula (I) containing a hydroxyl group that may be mentioned include the acetates, citrates, lactates, tartrates, malonates, oxalates, salicylates, propionates, succinates, fumarates, maleates, methylenebis-β-hydroxynaphthoates, gentisates, isethionates, di-p-tolyltartrates, methanesulfonates, ethanesulfonates, benzenesulfonates, p-toluenesulfonates, cyclohexylsulfamates and quinates.

Esters of products of Formula (I) that are particularly useful, containing a hydroxyl group, may be prepared from acid residues such as those described by Bundgaard et. al., J. Med. Chem., 1989, 32, page 2503-2507: these esters especially include substituted (aminomethyl)benzoates, dialkylaminomethylbenzoates in which the two alkyl groups may be linked together or may be interrupted with an oxygen atom or with an optionally substituted nitrogen atom, i.e., an alkylated nitrogen atom, or (morpholinomethyl)benzoates, e.g., 3- or 4-(morpholinomethyl)benzoates, and (4-alkylpiperazin-1-yl)benzoates, e.g., 3- or 4-(4-alkylpiperazin-1-yl)benzoates.

The carboxyl radical of the compound of Formula (I) may be salified or esterified with various groups known to those skilled in the art, among which nonlimiting examples include the following compounds:

among the salification compounds, mineral bases such as, for example, one equivalent of sodium, potassium, lithium, calcium, magnesium or ammonium, or organic bases such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine or N-methylglucamine;

among the esterification compounds, alkyl radicals to form alkoxycarbonyl groups such as, for example, methoxycarbonyl, ethoxycarbonyl, tert-butoxycarbonyl or benzyloxycarbonyl, these alkyl radicals possibly being substituted with radicals chosen, for example, from halogen atoms and hydroxyl, alkoxy, acyl, acyloxy, alkylthio, amino or aryl radicals, such as, for example, in chloromethyl, hydroxypropyl, methoxymethyl, propionyloxymethyl, methylthiomethyl, dimethylaminoethyl, benzyl or phenethyl groups.

The term “esterified carboxyl” means, for example, radical such as alkyloxycarbonyl radical, for example methoxycarbonyl, ethoxycarbonyl, propoxycarbonyl, butyl or tert-butyloxycarbonyl, cyclobutyloxycarbonyl, cyclopentyloxycarbonyl or cyclohexyloxycarbonyl.

Mention may also be made of radicals formed with readily cleavable ester residues, such as methoxymethyl or ethoxymethyl radicals; acyloxyalkyl radicals such as pivaloyloxymethyl, pivaloyloxyethyl, acetoxymethyl or acetoxyethyl; alkyloxycarbonyloxyalkyl radicals such as methoxycarbonyloxy methyl or ethyl radicals, and isopropyloxycarbonyloxy methyl or ethyl radicals.

A list of such ester radicals may be found, for example, in European patent EP 0 034 536.

The term “amidated carboxyl” means radical of the type —CONR5R6 as defined above: also intended are the radicals NCOR6R7 in which the radicals R6 and R7, which may be identical or different, are a hydrogen atom or an alkyl radical containing from 1 to 4 carbon atoms such as methyl, ethyl, propyl, isopropyl, butyl, isobutyl, sec-butyl or tert-butyl radicals and especially amino, alkylamino and dialkylamino radicals.

The term “alkylamino” means linear or branched methylamino, ethylamino, propylamino or butylamino radical. Alkyl radicals containing not more than 4 carbon atoms are preferred, the alkyl radicals possibly being chosen from the alkyl radicals mentioned above.

The term “dialkylamino” means, for example, dimethylamino, diethylamino and methylethylamino radical. As previously, alkyl radicals containing not more than 4 carbon atoms, chosen from the list indicated above, are preferred.

The radicals NR5R6 or NR6R7 may also form a heterocyclyl which may or may not comprise an additional hetero atom. Mention may be made of pyrrolyl, imidazolyl, indolyl, piperidyl, morpholinyl and piperazinyl radicals. The piperidyl, morpholinyl and piperazinyl radicals are preferred.

The term “salified carboxyl” means the salt formed, for example, with one equivalent of sodium, potassium, lithium, calcium, magnesium or ammonium. Mention may also be made of the salts formed with organic bases such as methylamine, propylamine, trimethylamine, diethylamine and triethylamine. The sodium salt is preferred.

When the compound of Formula (I) comprise an amino radical that may be salified with an acid, it is clearly understood that these acid salts also form part of the invention. Mention may be made of the salts obtained, for example, with hydrochloric acid or methanesulfonic acid.

The addition salts with mineral or organic acids of the compound of Formula (I) may be, for example, the salts formed with hydrochloric acid, hydrobromic acid, hydriodic acid, nitric acid, sulfuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulfonic acids such as, for example, methanesulfonic acid, ethanesulfonic acid or propanesulfonic acid, alkyldisulfonic acids such as, for example, methanedisulfonic acid or alpha,beta-ethanedisulfonic acid, arylmonosulfonic acids such as benzenesulfonic acid, and aryldisulfonic acids.

It may be recalled that stereoisomerism may be defined in its broad sense as the isomerism of compounds having the same structural formulae but whose various groups are arranged differently in space, especially such as in monosubstituted cyclohexanes whose substituent may be in an axial or equatorial position, and the various possible rotational conformations of ethane derivatives. However, there is another type of stereoisomerism, due to the different spatial arrangements of fixed substituents, either on double bonds or on rings, which is often referred to as geometrical isomerism or cis-trans (E and Z) isomerism. The term “stereoisomer” is used in the present patent application in its broadest sense and thus relates to all the compounds indicated above.

The Trk inhibitors of the present invention are cyclic urea compounds. The cyclic urea compounds are small molecules. The Trk inhibitors of the present invention can be administered, for example, orally, intravenously, intra-peritonally, intra-articularly and are particularly useful when administered locally to the site of desired action.

This invention relates to methods of inhibiting Trk with cyclic urea compounds and methods of treating disease by with cyclic urea compounds. The invention also pertains to methods of treating OA, methods of treating pain, and method of treating pain associated with OA with cyclic urea compounds. The cyclic urea compounds and pharmaceutical compositions comprising cyclic urea compounds can be administered in multiple dosage forms, including an injection for local delivery. The cyclic urea compounds are the active pharmaceutical ingredient in pharmaceutical compositions comprising cyclic urea compounds; the cyclic urea compounds can also be co-administered and/or co-formulated with other active ingredients for the treatment of disease, including OA and pain associated with OA.

The present invention relates to a compound with the structure of Formula (I):

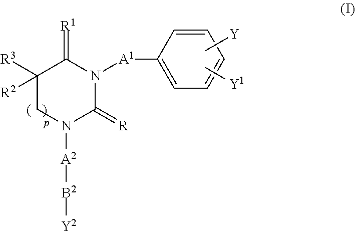

wherein:
p is 0, 1 or 2;
R and R¹, which may be identical or different, are O or NH;
R² and R³, which may be identical or different, are hydrogen, alkyl, alkenyl, alkynyl, cycloalkyl, aryl and heteroaryl that are optionally substituted, or R² and R³ taken together with the carbon atom to which they are attached form 3- to 10-membered carbocyclyl that is optionally being substituted or 3- to 10-membered heterocyclyl containing one or more hetero atoms chosen from O, S, N and NR⁷ that is optionally being substituted;
A¹ is single bond, alkyl, alkenyl or alkynyl;
Y and Y¹, which may be identical or different, are such that one of Y and Y is —OCF₃, —O—F₂—CHF₂, —O—CHF₂, —O—CH₂—CF₃, —SO₂NR⁵R⁶, —SF₅ and —S(O)ₙ-alkyl and the other of Y and Y¹ is —OCF₃, —O—F₂—CHF₂, —O—CHF₂, —O—CH₂—CF₃, SO₂NR⁵R⁶, —SF₅, —S(O)ₙ-alkyl, hydrogen, halogen, hydroxyl, alkoxy, nitro, —CN, —NR⁵R⁶, optionally substituted alkyl, optionally substituted aryl, optionally substituted heteroaryl, —CF₃, —O-alkenyl, —O-alkynyl, —O-cycloalkyl, —S(O)ₙ-alkenyl, —S(O)ₙ-alkynyl, —S(O)ₙ-cycloalkyl, —CONR⁵R⁶, or free, salified or esterified carboxyl, or the

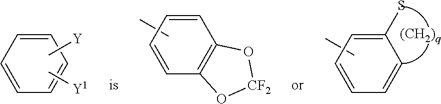

that is optionally substituted with one or more alkyl that are optionally substituted, wherein
   q is 2, 3 or 4;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
A², which may be identical to or different from A¹, is defined as A¹ or is CO or SO₂,
B² is saturated or unsaturated, 3- to 10-membered monocyclic or bicyclic heterocyclyl containing one or more hetero atoms chosen from O, S, N and NR⁷ that is optionally substituted with one or more identical or different substituents defined as Y², wherein
   R⁷ is hydrogen, alkyl, cycloalkyl, phenyl, acyl, —S(O)₂Alk, —S(O)₂Aryl, —S(O)₂heteroaryl or —S(O)₂NR⁵R⁶,
   Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹⁰—CS—NR⁵R⁶ or —NR¹⁰—COOR¹³, all of which are optionally substituted;
all the alkyl, alkenyl, alkynyl and alkoxy above are linear or branched and contain not more than 6 carbon atoms,
all the cycloalkyl and heterocyclyl above contain not more than 7 carbon atoms,
all the aryl and heteroaryl above contain not more than 10 carbon atoms,
all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²,
all the aryl and heteroaryl above are optionally substituted with one or more substituents selected from the group consisting of alkyl, alkoxy and alkylenedioxy,
all the cyclic radicals above, and also the ring formed by R⁵ and R⁶ with the nitrogen atom to which they are attached, are optionally substituted with one or more substituents selected from the group consisting of oxo and thioxo,
n is 0 to 2, and wherein
   R⁸ is alkyl, alkenyl, cycloalkyl, cycloalkylalkyl, heterocyclyl, heterocyclylalkyl, aryl, arylalkyl, heteroaryl and heteroarylalkyl,
   R⁹ is defined as R⁸ or is hydrogen,
   R¹⁰ is hydrogen or alkyl,
   R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl, or R¹¹ and R¹² taken together with the nitrogen atom to which they are attached form 5- to 7-membered cyclic radical containing one or more hetero atoms chosen from O, S, N and NR⁷, preferably a cyclic amine, and
   R¹³, which may be identical to or different to R⁵ or R⁶, is defined as R⁵ or R⁶, or
      a racemic, enantiomeric or diastereoisomeric isomer form of the compound of Formula (I), addition salt with mineral or organic acid or with mineral or organic base thereof, with the proviso:
a) when p is 0, R and R¹ are oxygen, A¹ is single bond or alkyl, Y and Y¹, which may be identical or different, are at least one is —OCF₃ or —S-alk, A² is single bond or alkyl and B² is an optionally substituted heterocyclyl, then R² and R³ are not one hydrogen and the other imidazolylalkyl;
b) when p is 0, R and R¹ are oxygen, A¹ is single bond or alkyl, Y and Y¹, which may be identical or different, are at least one is —OCF₃, —SO-Alk, —S(O)₂-alk or —SO₂NH₂, A² is CH₂ and B² is an optionally substituted heterocyclyl, then R² and R³ are not one hydrogen and the other alkyl optionally interrupted with O, S or N-alk; always substituted with a hydroxamate (—CO—NHOH);
c) when p is 0, R and R¹ are oxygen, A¹ is a single bond or alkyl, Y and Y¹, which may be identical or different, are at least one is —S(O)ₙ-alk, A² is single bond and B² is an optionally substituted 5- or 6-membered aromatic heterocyclyl, then R² and R³ are not selected from the group consisting of hydrogen, alkyl, arylalkyl, aryl and heteroaryl; or
d) when p is 0 to 2, R and R¹ are oxygen, A¹ is single bond, Y and Y¹, which may be identical or different, are one is —SO₂Alk or SO₂NH₂ and the other is NR⁵R⁶, A² is single bond or alkylene and B² is optionally substituted 5- to 10-membered heterocyclyl, then R² and R³ are not both hydrogen.

In a preferred embodiment, the present invention relates to a compound of Formula (I), with the structure of Formula (II):

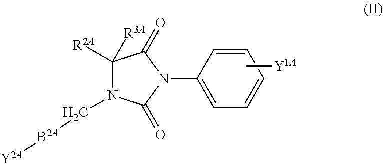

wherein:
Y^{1A} is —OCF₃, —S(O)ₙ—CF₃ and —SO₂CHF₂;
B^{2A} is 4-quinolyl and 4-pyridyl optionally substituted with one or more radicals chosen from the definition of Y^{2A};
Y^{2A} is defined as Y²;
R^{2A} and R^{3A} taken together with the carbon atom to which they are attached form a C₃-C₁₀ cycloalkyl or heterocyclyl,
all the alkyl and phenyl above are optionally substituted with one or more radicals chosen from halogen, —OH, alk, —O-alk, —OCF₃, —S(O)ₙ—CF₃, —CF₃, —NH₂, —NH-Alk and —N(Alk)₂ and
Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹⁰—CS—NR⁵R⁶ or —NR¹⁰—COOR¹³, all of which are optionally substituted;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
R⁷ is hydrogen, alkyl, cycloalkyl, phenyl, acyl, —S(O)₂Alk, —S(O)₂Aryl, —S(O)₂heteroaryl or —S(O)₂NR⁵R⁶,
except for Y^{1A}, B^{2A}, R^{2A} and R^{3A}, all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²,
except for B^{2A}, R^{2A} and R^{3A}, all the cyclic radicals above, and also the ring formed by R⁵ and R⁶ with the nitrogen atom to which they are attached, are optionally substituted with one or more substituents selected from the group consisting of oxo and thioxo,
R⁸ is alkyl, alkenyl, cycloalkyl, cycloalkylalkyl, heterocyclyl, heterocyclylalkyl, aryl, arylalkyl, heteroaryl and heteroarylalkyl,
R⁹ is defined as R⁸ or is hydrogen,
R¹⁰ is hydrogen or alkyl,
R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl,
or R¹¹ and R¹² taken together with the nitrogen atom to which they are attached form 5- to 7-membered cyclic radical containing one or more hetero atoms chosen from O, S, N and NR⁷, R¹³, which may be identical to or different to R⁵ or R⁶, is defined as R⁵ or R⁶, and
n is O to 2; or
a racemic, enantiomeric or diastereoisomeric isomer form of said compound, or addition salt with a mineral or organic acid or with mineral or organic base thereof.

In a preferred embodiment, the present invention relates to a compound of Formula (I), wherein the compound is 1-(4-((5,5-dimethyl-2,4-dioxo-3-(4-((trifluoromethyl)thio)phenyl)imidazolidin-1-yl)methyl)pyridin-2-yl)-3-(3-(dimethylamino)propyl)urea or 5,5-dimethyl-1-((2-(pyridin-2-ylamino)pyridin-4-yl)methyl)-3-(4-((trifluoromethyl)thio)phenyl)imidazolidine-2,4-dione.

In a preferred embodiment, the present invention relates to a compound of Formula (I), methods of treating osteoarthritis, methods of treating pain, and methods of treating pain associated with osteoarthritis comprising administering a compound of Formula (I), and methods of inhibiting tropoymyosin receptor kinase A, methods of inhibiting tropoymyosin receptor kinase B, and methods of inhibiting tropoymyosin receptor kinase C, wherein B² is a pyridyl radical or a quinolyl radical.

In another preferred embodiment, the present invention relates to methods of treating osteoarthritis, methods of treating pain, and methods of treating pain associated with osteoarthritis comprising administering a compound of Formula (I), and methods of inhibiting tropoymyosin receptor kinase A, methods of inhibiting tropoymyosin receptor kinase B, and methods of inhibiting tropoymyosin receptor kinase C with 1-(4-((5,5-dimethyl-2,4-dioxo-3-(4-((trifluoromethyl)thio)phenyl)imidazolidin-1-yl)methyl)pyridin-2-yl)-3-(3-(dimethylamino)propyl)urea or 5,5-dimethyl-1-((2-(pyridin-2-ylamino)pyridin-4-yl)methyl)-3-(4-((trifluoromethyl)thio)phenyl)imidazolidine-2,4-dione.

The compounds of Table 1 may be synthesized according to the processes described in the examples for obtaining the compound of Formula (I), and especially according to the operating conditions described for the preparation of Example 1-61 to Example 1-63.

TABLE 1
Compounds of Formula (I)
Wherein B² is a Pyridyl Radical or a Quinolyl Radical

| | |
|---|---|
| 5,5-dimethyl-1-(2-methylpyrid-4-ylmethyl)-3-(4- | 5,5-dimethyl-1-(2-ethylpyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(2-isopropylpyrid-4-ylmethyl)-3-(4- | 5,5-dimethyl-1-(2-tert-butylpyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(2-isobutylpyrid-4-ylmethyl)-3-(4- | 5,5-dimethyl-1-(2-cyclohexylpyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(2-phenylpyrid-4-ylmethyl)-3-(4- | 5,5-dimethyl-1-[2-(4-chlorophenyl)pyrid-4-ylmethyl]- |
| trifluoromethoxyphenyl)imidazolidine-2,4-dione | 3-(4-trifluoromethoxyphenyl)imidazolidine-2,4-dione |
| 5,5-dimethyl-1-[2-(3-chlorophenyl)pyrid-4- | 5,5-dimethyl-1-[2-(2-chlorophenyl)pyrid-4-ylmethyl]- |
| ylmethyl]-3-(4- | 3-(4-trifluoromethoxyphenyl)imidazolidine-2,4-dione |
| trifluoromethoxyphenyl)imidazolidine-2,4-dione | |
| 5,5-dimethyl-1-[2-(4-fluorophenyl)pyrid-4- | 5,5-dimethyl-1-[2-(4-methoxyphenyl)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-(2-benzylpyrid-4-ylmethyl)-3-(4- | 5,5-dimethyl-1-(2,2′-bipyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(2-hydroxymethylpyrid-4-ylmethyl)- | 5,5-dimethyl-1-(2-chloropyrid-4-ylmethyl)-3-(4- |
| 3-(4-trifluoromethoxyphenyl)imidazolidine-2,4- | trifluoromethoxyphenyl)imidazolidine-2,4-dione |
| dione | |
| 5,5-dimethyl-1-(2-fluoropyrid-4-ylmethyl)-3-(4- | 5,5-dimethyl-1-(2-bromopyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(2-aminopyrid-4-ylmethyl)-3-(4- | 5,5-dimethyl-1-(2-methylaminopyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(2-ethylaminopyrid-4-ylmethyl)-3- | 5,5-dimethyl-1-(2-isopropylaminopyrid-4-ylmethyl)-3- |
| (4-trifluoromethoxyphenyl)imidazolidine-2,4-dione | (4-trifluoromethoxyphenyl)imidazolidine-2,4-dione |
| 5,5-dimethyl-1-(2-phenylaminopyrid-4-ylmethyl)-3- | 5,5-dimethyl-1-(2-cyclohexylaminopyrid-4-ylmethyl)- |
| (4-trifluoromethoxyphenyl)imidazolidine-2,4-dione | 3-(4-trifluoromethoxyphenyl)imidazolidine-2,4-dione |
| 5,5-dimethyl-1-(2-diethylaminopyrid-4-ylmethyl)-3- | 5,5-dimethyl-1-(2-N-cyclohexyl-N-methylaminopyrid- |
| (4-trifluoromethoxyphenyl)imidazolidine-2,4-dione | 4-ylmethyl)-3-(4- |
| | trifluoromethoxyphenyl)imidazolidine-2,4-dione |
| 5,5-dimethyl-1-(2-piperidinopyrid-4-ylmethyl)-3-(4- | 5,5-dimethyl-1-(2-morpholinopyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-[2-(4-methylpiperazino)pyrid-4- | 5,5-dimethyl-1-[2-(1H-pyrazol-1-yl)pyrid-4-ylmethyl]- |
| ylmethyl]-3-(4- | 3-(4-trifluoromethoxyphenyl)imidazolidine-2,4-dione |
| trifluoromethoxyphenyl)imidazolidine-2,4-dione | |
| 5,5-dimethyl-1-[2-(2-benzoylhydrazino)pyrid-4- | 5,5-dimethyl-1-(2-benzoylaminopyrid-4-ylmethyl)-3- |
| ylmethyl]-3-(4- | (4-trifluoromethoxyphenyl)imidazolidine-2,4-dione |
| trifluoromethoxyphenyl)imidazolidine-2,4-dione | |
| 5,5-dimethyl-1-[2-(4-chlorobenzoylamino)pyrid-4- | 5,5-dimethyl-1-[2-(3-chlorobenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-[2-(2-chlorobenzoylamino)pyrid-4- | 5,5-dimethyl-1-[2-(4-fluorobenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-[2-(4-methoxybenzoylamino)pyrid- | 5,5-dimethyl-1-(2-acetamidopyrid-4-ylmethyl)-3-(4- |
| 4-yl-methyl]-3-(4- | trifluoromethoxyphenyl)imidazolidine-2,4-dione |
| trifluoromethoxyphenyl)imidazolidine-2,4-dione | |
| 5,5-dimethyl-1-(2-phenylacetamidopyrid-4- | 5,5-dimethyl-1-[2-(4- |
| ylmethyl)-3-(4- | methylpiperazino)acetamidopyrid-4-ylmethyl]-3-(4- |
| trifluoromethoxyphenyl)imidazolidine-2,4-dione | trifluoromethoxyphenyl)imidazolidine-2,4-dione |
| 5,5-dimethyl-1-(2-cyclohexanylcarboxamidopyrid- | 5,5-dimethyl-1-[2-(1-methyl-4-piperidinyl)carbonyl- |
| 4-ylmethyl)-3-(4- | aminopyrid-4-ylmethyl]-3-(4-trifluoromethoxyphenyl)- |
| trifluoromethoxyphenyl)imidazolidine-2,4-dione | imidazolidine-2,4-dione |
| 5,5-dimethyl-1-[2-(2-furanylcarbonyl)aminopyrid-4- | 5,5-dimethyl-1-(2-ethoxycarbonylaminopyrid-4- |
| ylmethyl]-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-(2-isopropoxycarbonylaminopyrid- | 5,5-dimethyl-1-(2-butoxycarbonylaminopyrid-4- |
| 4-ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-[2-(2-diethylaminoethoxy- | 5,5-dimethyl-1-(2-phenoxycarbonylaminopyrid-4-yl- |
| carbonylamino)pyrid-4-ylmethyl]-3-(4- | methyl)-3-(4-trifluoromethoxyphenyl)imidazolidine- |
| trifluoromethoxyphenyl)imidazolidine-2,4-dione | 2,4-dione |
| 5,5-dimethyl-1-[2-(4- | 5,5-dimethyl-1-(2-anilinocarbonylaminopyrid-4- |
| chloroanilino)carbonylaminopyrid-4-ylmethyl]-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-(2-benzylaminocarbonylaminopyrid- | 5,5-dimethyl-1-[2-(2- |
| 4-ylmethyl)-3-(4- | pyridylamino)carbonylaminopyrid-4-ylmethyl]-3-(4- |
| trifluoromethoxyphenyl)imidazolidine-2,4-dione | trifluoromethoxyphenyl)imidazolidine-2,4-dione |
| 5,5-dimethyl-1-(2-ureidopyrid-4-ylmethyl)-3-(4- | 5,5-dimethyl-1-(2-benzylaminosulfonylaminopyrid-4- |
| trifluoromethoxyphenyl)imidazolidine-2,4-dione | ylmethyl)-3-(4-trifluoromethoxyphenyl)imidazolidine- |
| | 2,4-dione |
| 5,5-dimethyl-1-(2-methylaminosulfonylaminopyrid- | 5,5-dimethyl-1-(2-aminosulfonylaminopyrid-4- |
| 4-ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-(2-morpholinosulfonylaminopyrid-4- | 5,5-dimethyl-1-(2-dimethylaminosulfonylaminopyrid- |
| ylmethyl)-3-(4- | 4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(2-phenylsulfonylaminopyrid-4- | 5,5-dimethyl-1-[2-(2-thienyl)sulfonylaminopyrid-4-yl- |
| ylmethyl)-3-(4- | methyl]-3-(4-trifluoromethoxyphenyl)imidazolidine- |
| trifluoromethoxyphenyl)imidazolidine-2,4-dione | 2,4-dione |
| 5,5-dimethyl-1-(2-methylsulfonylaminopyrid-4- | 5,5-dimethyl-1-[2-(4-chlorophenyl)sulfonylaminopyrid- |
| ylmethyl)-3-(4- | 4-ylmethyl]-3-(4- |
| trifluoromethoxyphenyl)imidazolidine-2,4-dione | trifluoromethoxyphenyl)imidazolidine-2,4-dione |
| 5,5-dimethyl-1-(2-benzylsulfonylaminopyrid-4- | 5,5-dimethyl-1-(2-hydroxycarbonylpyrid-4-ylmethyl)- |
| ylmethyl)-3-(4- | 3-(4-trifluoromethoxyphenyl)imidazolidine-2,4-dione |
| trifluoromethoxyphenyl)imidazolidine-2,4-dione | |
| 5,5-dimethyl-1-(2-ethoxycarbonylpyrid-4-ylmethyl)- | 5,5-dimethyl-1-(2-tert-butoxycarbonylpyrid-4- |
| 3-(4-trifluoromethoxyphenyl)imidazolidine-2,4- | ylmethyl)-3-(4-trifluoromethoxyphenyl)imidazolidine- |
| dione | 2,4-dione |
| 5,5-dimethyl-1-(2-phenoxycarbonyrpyrid-4- | 5,5-dimethyl-1-[2-(4-methylpiperazinocarbonyl)pyrid- |
| ylmethyl)-3-(4- | 4-ylmethyl]-3-(4- |
| trifluoromethoxyphenyl)imidazolidine-2,4-dione | trifluoromethoxyphenyl)imidazolidine-2,4-dione |
| 5,5-dimethyl-1-[2-(methylamino)carbonyrpyrid-4- | 5,5-dimethyl-1-[2-(dimethylamino)carbonylpyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-(2-piperidinocarbonylpyrid-4- | 5,5-dimethyl-1-(2-morpholinocarbonylpyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-[2-(4- | 5,5-dimethyl-1-(2-phenylaminocarbonylpyrid-4- |
| phenylpiperazinocarbonyl)pyrid-4-ylmethyl]-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-[2- | 5,5-dimethyl-1-[2-(4- |
| (methylphenylamino)carbonylpyrid-4-ylmethyl]-3- | methoxyphenyl)methylaminocarbonylpyrid-4- |
| (4-trifluoromethoxyphenyl)imidazolidine-2,4-dione | ylmethyl]-3-(4-trifluoromethoxyphenyl)imidazolidine- |
| | 2,4-dione |
| 5,5-dimethyl-1-[2-(4- | 5,5-dimethyl-1-[2-(4-morpholinylamino)carbonylpyrid- |
| chlorophenyl)methylaminocarbonylpyrid-4- | 4-ylmethyl]-3-(4- |
| ylmethyl]-3-(4-trifluoromethoxyphenyl)imidazol- | trifluoromethoxyphenyl)imidazolidine-2,4-dione |
| idine-2,4-dione | |
| 5,5-dimethyl-1-(2-aminocarbonylpyrid-4-ylmethyl)- | 5,5-dimethyl-1-(2-phenoxypyrid-4-ylmethyl)-3-(4- |
| 3-(4-trifluoromethoxyphenyl)imidazolidine-2,4- | trifluoromethoxyphenyl)imidazolidine-2,4-dione |
| dione | |
| 5,5-dimethyl-1-(2-methoxypyrid-4-ylmethyl)-3-(4- | 5,5-dimethyl-1-(2-ethoxypyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-[2-(1-pyrrolidinyl)ethoxypyrid-4-yl- | 5,5-dimethyl-1-(2-benzyloxypyrid-4-ylmethyl)-3-(4- |
| methyl]-3-(4-trifluoromethoxyphenyl)imidazolidine- | trifluoromethoxyphenyl)imidazolidine-2,4-dione |
| 2,4-dione | |
| 5,5-dimethyl-1-(2-benzoyloxypyrid-4-ylmethyl)-3- | 5,5-dimethyl-1-[2-(4,5-dihydro-1H-imidazol-2- |
| (4-trifluoromethoxyphenyl)imidazolidine-2,4-dione | yl)pyrid-4-ylmethyl]-3-(4- |
| | trifluoromethoxyphenyl)imidazolidine-2,4-dione |
| 5,5-dimethyl-1-[2-(1H-imidazol-2-yl)pyrid-4- | 5,5-dimethyl-1-[2-(1H-tetrazol-5-yl)pyrid-4-ylmethyl]- |
| ylmethyl]-3-(4- | 3-(4-trifluoromethoxyphenyl)imidazolidine-2,4-dione |
| trifluoromethoxyphenyl)imidazolidine-2,4-dione | |
| 5,5-dimethyl-1-(2-amidinopyrid-4-ylmethyl)-3-(4- | 5,5-dimethyl-1-(2-dimethylaminomethylpyrid-4- |
| trifluoromethoxyphenyl)imidazolidine-2,4-dione | ylmethyl)-3-(4-trifluoromethoxyphenyl)imidazolidine- |
| | 2,4-dione |
| 5,5-dimethyl-1-(2-aminomethylpyrid-4-ylmethyl)-3- | 5,5-dimethyl-1-(3-methylpyrid-4-ylmethyl)-3-(4- |
| (4-trifluoromethoxyphenyl)imidazolidine-2,4-dione | trifluoromethoxyphenyl)imidazolidine-2,4-dione |
| 5,5-dimethyl-1-(3-ethylpyrid-4-ylmethyl)-3-(4- | 5,5-dimethyl-1-(3-isopropylpyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(3-tert-butylpyrid-4-ylmethyl)-3-(4- | 5,5-dimethyl-1-(3-isobutylpyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(3-cyclohexylpyrid-4-ylmethyl)-3- | 5,5-dimethyl-1-(3-phenylpyrid-4-ylmethyl)-3-(4- |
| (4-trifluoromethoxyphenyl)imidazolidine-2,4-dione | trifluoromethoxyphenyl)imidazolidine-2,4-dione |
| 5,5-dimethyl-1-[3-(4-chlorophenyl)pyrid-4- | 5,5-dimethyl-1-[3-(3-chlorophenyl)pyrid-4-ylmethyl]- |
| ylmethyl]-3-(4- | 3-(4-trifluoromethoxyphenyl)imidazolidine-2,4-dione |
| trifluoromethoxyphenyl)imidazolidine-2,4-dione | |
| 5,5-dimethyl-1-[3-(2-chlorophenyl)pyrid-4- | 5,5-dimethyl-1-[3-(4-fluorophenyl)pyrid-4-ylmethyl]-3- |
| ylmethyl]-3-(4- | (4-trifluoromethoxyphenyl)imidazolidine-2,4-dione |
| trifluoromethoxyphenyl)imidazolidine-2,4-dione | |
| 5,5-dimethyl-1-[3-(4-methoxyphenyl)pyrid-4- | 5,5-dimethyl-1-(3-benzylpyrid-4-ylmethyl)-3-(4- |
| ylmethyl]-3-(4- | trifluoromethoxyphenyl)imidazolidine-2,4-dione |
| trifluoromethoxyphenyl)imidazolidine-2,4-dione | |
| 5,5-dimethyl-1-(3,2′-bipyrid-4-ylmethyl)-3-(4- | 5,5-dimethyl-1-(3-hydroxymethylpyrid-4-ylmethyl)-3- |
| trifluoromethoxyphenyl)imidazolidine-2,4-dione | (4-trifluoromethoxyphenyl)imidazolidine-2,4-dione |
| 5,5-dimethyl-1-(3-chloropyrid-4-ylmethyl)-3-(4- | 5,5-dimethyl-1-(3-fluoropyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(3-bromopyrid-4-ylmethyl)-3-(4- | 5,5-dimethyl-1-(3-aminopyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-(3-methylaminopyrid-4-ylmethyl)-3- | 5,5-dimethyl-1-(3-ethylaminopyrid-4-ylmethyl)-3-(4- |
| (4-trifluoromethoxyphenyl)imidazolidine-2,4-dione | trifluoromethoxyphenyl)imidazolidine-2,4-dione |
| 5,5-dimethyl-1-(3-isopropylaminopyrid-4- | 5,5-dimethyl-1-(3-phenylaminopyrid-4-ylmethyl)-3-(4- |
| ylmethyl)-3-(4- | trifluoromethoxyphenyl)imidazolidine-2,4-dione |
| trifluoromethoxyphenyl)imidazolidine-2,4-dione | |
| 5,5-dimethyl-1-(3-cyclohexylaminopyrid-4- | 5,5-dimethyl-1-(3-diethylaminopyrid-4-ylmethyl)-3-(4- |
| ylmethyl)-3-(4- | trifluoromethoxyphenyl)imidazolidine-2,4-dione |
| trifluoromethoxyphenyl)imidazolidine-2,4-dione | |
| 5,5-dimethyl-1-(3-N-cyclohexyl-N- | 5,5-dimethyl-1-(3-piperidinopyrid-4-ylmethyl)-3-(4- |
| methylaminopyrid-4-ylmethyl)-3-(4- | trifluoromethoxyphenyl)imidazolidine-2,4-dione |
| trifluoromethoxyphenyl)imidazolidine-2,4-dione | |
| 5,5-dimethyl-1-(3-morpholinopyrid-4-ylmethyl)-3- | 5,5-dimethyl-1-[3-(4-methylpiperazino)pyrid-4- |
| (4-trifluoromethoxyphenyl)imidazolidine-2,4-dione | ylmethyl]-3-(4-trifluoromethoxyphenyl)imidazolidine- |
| | 2,4-dione |
| 5,5-dimethyl-1-[3-(1H-pyrazol-1-yl)pyrid-4- | 5,5-dimethyl-1-[3-(2-benzoylhydrazino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-(3-benzoylaminopyrid-4-ylmethyl)- | 5,5-dimethyl-1-[3-(4-chlorobenzoylamino)pyrid-4- |
| 3-(4-trifluoromethoxyphenyl)imidazolidine-2,4- | ylmethyl]-3-(4-trifluoromethoxyphenyl)imidazolidine- |
| dione | 2,4-dione |
| 5,5-dimethyl-1-[3-(3-chlorobenzoylamino)pyrid-4- | 5,5-dimethyl-1-[3-(2-chlorobenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-[3-(4-fluorobenzoylamino)pyrid-4- | 5,5-dimethyl-1-[3-(4-methoxybenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-(3-acetamidopyrid-4-ylmethyl)-3-(4- | 5,5-dimethyl-1-(3-phenylacetamidopyrid-4-ylmethyl)- |
| trifluoromethoxyphenyl)imidazolidine-2,4-dione | 3-(4-trifluoromethoxyphenyl)imidazolidine-2,4-dione |
| 5,5-dimethyl-1-[3-(4- | 5,5-dimethyl-1-(3-cyclohexanylcarboxamidopyrid-4- |
| methylpiperazino)acetamidopyrid-4-ylmethyl]-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-[3-(1-methyl-4-piperidinyl)carbonyl- | 5,5-dimethyl-1-[3-(2-furanylcarbonyl)aminopyrid-4- |
| aminopyrid-4-ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-(3-ethoxycarbonylaminopyrid-4- | 5,5-dimethyl-1-(3-isopropoxycarbonylaminopyrid-4-yl- |
| ylmethyl)-3-(4- | methyl)-3-(4-trifluoromethoxyphenyl)imidazolidine- |
| trifluoromethoxyphenyl)imidazolidine-2,4-dione | 2,4-dione |
| 5,5-dimethyl-1-(3-butoxycarbonylaminopyrid-4- | 5,5-dimethyl-1-[3-(2-diethylaminoethoxycarbonyl- |
| ylmethyl)-3-(4- | amino)pyrid-4-ylmethyl]-3-(4- |
| trifluoromethoxyphenyl)imidazolidine-2,4-dione | trifluoromethoxyphenyl)imidazolidine-2,4-dione |
| 5,5-dimethyl-1-(3-phenoxycarbonylaminopyrid-4- | 5,5-dimethyl-1-[3-(4- |
| ylmethyl)-3-(4- | chloroanilino)carbonylaminopyrid-4-ylmethyl]-3-(4- |
| trifluoromethoxyphenyl)imidazolidine-2,4-dione | trifluoromethoxyphenyl)imidazolidine-2,4-dione |
| 5,5-dimethyl-1-(3-anilinocarbonylaminopyrid-4- | 5,5-dimethyl-1-(3-benzylaminocarbonylaminopyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-[3-(2- | 5,5-dimethyl-1-(3-ureidopyrid-4-ylmethyl)-3-(4- |
| pyridylamino)carbonylaminopyrid-4-ylmethyl]-3-(4- | trifluoromethoxyphenyl)imidazolidine-2,4-dione |
| trifluoromethoxyphenyl)imidazolidine-2,4-dione | |
| 5,5-dimethyl-1-(3-benzylaminosulfonylaminopyrid- | 5,5-dimethyl-1-(3-methylaminosulfonylaminopyrid-4- |
| 4-ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-(3-aminosulfonylaminopyrid-4- | 5,5-dimethyl-1-(3-morpholinosulfonylaminopyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-(3- | 5,5-dimethyl-1-(3-anilinosulfonylaminopyrid-4- |
| dimethylaminosulfonylaminopyrid-4-ylmethyl)-3- | ylmethyl)-3-(4-trifluoromethoxyphenyl)imidazolidine- |
| (4-trifluoromethoxyphenyl)imidazolidine-2,4-dione | 2,4-dione |
| 5,5-dimethyl-1-(3-phenylsulfonylaminopyrid-4- | 5,5-dimethyl-1-[3-(2-thienyl)sulfonylaminopyrid-4- |
| ylmethyl)-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-(3-methylsulfonylaminopyrid-4- | 5,5-dimethyl-1-[3-(4-chlorophenyl)sulfonylaminopyrid- |
| ylmethyl)-3-(4- | 4-ylmethyl]-3-(4- |
| trifluoromethoxyphenyl)imidazolidine-2,4-dione | trifluoromethoxyphenyl)imidazolidine-2,4-dione |
| 5,5-dimethyl-1-(3-benzylsulfonylaminopyrid-4- | 5,5-dimethyl-1-(3-hydroxycarbonylpyrid-4-ylmethyl)- |
| ylmethyl)-3-(4- | 3-(4-trifluoromethoxyphenyl)imidazolidine-2,4-dione |
| trifluoromethoxyphenyl)imidazolidine-2,4-dione | |
| 5,5-dimethyl-1-(3-ethoxycarbonylpyrid-4-ylmethyl)- | 5,5-dimethyl-1-(3-tert-butoxycarbonylpyrid-4- |
| 3-(4-trifluoromethoxyphenyl)imidazolidine-2,4- | ylmethyl)-3-(4-trifluoromethoxyphenyl)imidazolidine- |
| dione | 2,4-dione |
| 5,5-dimethyl-1-(3-phenoxycarbonylpyrid-4- | 5,5-dimethyl-1-[3-(4-methylpiperazinocarbonyl)pyrid- |
| ylmethyl)-3-(4- | 4-ylmethyl]-3-(4- |
| trifluoromethoxyphenyl)imidazolidine-2,4-dione | trifluoromethoxyphenyl)imidazolidine-2,4-dione |
| 5,5-dimethyl-1-[3-(methylamino)carbonylpyrid-4- | 5,5-dimethyl-1-[3-(dimethylamino)carbonylpyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5,5-dimethyl-1-(3-piperidinocarbonylpyrid-4- | 5,5-dimethyl-1-(3-morpholinocarbonylpyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-[3-(4- | 5,5-dimethyl-1-(3-phenylaminocarbonylpyrid-4- |
| phenylpiperazinocarbonyl)pyrid-4-ylmethyl]-3-(4- | ylmethyl)-3-(4-trifluoromethoxyphenyl)imidazolidine- |
| trifluoromethoxyphenyl)imidazolidine-2,4-dione | 2,4-dione |
| 5,5-dimethyl-1-[3- | 5,5-dimethyl-1-[3-(4- |
| (methylphenylamino)carbonylpyrid-4-ylmethyl]-3- | methoxyphenyl)methylaminocarbonylpyrid-4- |
| (4-trifluoromethoxyphenyl)imidazolidine-2,4-dione | ylmethyl]-3-(4-trifluoromethoxyphenyl)imidazolidine- |
| | 2,4-dione |
| 5,5-dimethyl-1-[3-(4- | 5,5-dimethyl-1-[3-(4-morpholinylamino)carbonylpyrid- |
| chlorophenyl)methylaminocarbonyrpyrid-4- | 4-ylmethyl]-3-(4- |
| ylmethyl]-3-(4-trifluoromethoxyphenyl)imidazol- | trifluoromethoxyphenyl)imidazolidine-2,4-dione |
| idine-2,4-dione | |
| 5,5-dimethyl-1-(3-aminocarbonylpyrid-4-ylmethyl)- | 5,5-dimethyl-1-(3-phenoxypyrid-4-ylmethyl)-3-(4- |
| 3-(4-trifluoromethoxyphenyl)imidazolidine-2,4- | trifluoromethoxyphenyl)imidazolidine-2,4-dione |
| dione | |
| 5,5-dimethyl-1-(3-methoxypyrid-4-ylmethyl)-3-(4- | 5,5-dimethyl-1-(3-ethoxypyrid-4-ylmethyl)-3-(4- |
| trifluoromethoxyphenyl)imidazolidine-2,4-dione | trifluoromethoxyphenyl)imidazolidine-2,4-dione |
| 5,5-dimethyl-1-[3-(1-pyrrolidinyl)ethoxypyrid-4-yl- | 5,5-dimethyl-1-(3-benzyloxypyrid-4-ylmethyl)-3-(4- |
| methyl]-3-(4-trifluoromethoxyphenyl)imidazolidine- | trifluoromethoxyphenyl)imidazolidine-2,4-dione |
| 2,4-dione | |
| 5,5-dimethyl-1-(3-benzoyloxypyrid-4-ylmethyl)-3- | 5,5-dimethyl-1-[3-(4,5-dihydro-1H-imidazol-2- |
| (4-trifluoromethoxyphenyl)imidazolidine-2,4-dione | yl)pyrid-4-ylmethyl]-3-(4- |
| | trifluoromethoxyphenyl)imidazolidine-2,4-dione |
| 5,5-dimethyl-1-[3-(1H-imidazol-2-yl)pyrid-4- | 5,5-dimethyl-1-[3-(1H-tetrazol-5-yl)pyrid-4-ylmethyl]- |
| ylmethyl]-3-(4- | 3-(4-trifluoromethoxyphenyl)imidazolidine-2,4-dione |
| trifluoromethoxyphenyl)imidazolidine-2,4-dione | |
| 5,5-dimethyl-1-(3-amidinopyrid-4-ylmethyl)-3-(4- | 5,5-dimethyl-1-(3-dimethylaminomethylpyrid-4- |
| trifluoromethoxyphenyl)imidazolidine-2,4-dione | ylmethyl)-3-(4-trifluoromethoxyphenyl)imidazolidine- |
| | 2,4-dione |
| 5,5-dimethyl-1-(3-aminomethylpyrid-4-ylmethyl)-3- | 4-(2-methylpyridin)-4-ylmethyl-6-(4- |
| (4-trifluoromethoxyphenyl)imidazolidine-2,4-dione | trifluoromethoxyphenyl)-4,6-diazaspiro[2.4]heptane- |
| | 5,7-dione |
| 4-(2-ethylpyridin)-4-ylmethyl-6-(4- | 4-(2-isopropylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-tert-butylpyridin)-4-ylmethyl-6-(4- | 4-(2-isobutylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-cyclohexylpyridin)-4-ylmethyl-6-(4- | 4-(2-phenylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(4-chlorophenyl)pyridin]-4-ylmethyl-6-(4- | 4-[2-(3-chlorophenyl)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(2-chlorophenyl)pyridin]-4-ylmethyl-6-(4- | 4-[2-(4-fluorophenyl)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(4-methoxyphenyl)pyridin]-4-ylmethyl-6-(4- | 4-(2-benzylpyridin)-4-ylmethyl-6-(4-trifluoromethoxy- |
| trifluoromethoxyphenyl)-4,6- | phenyl)-4,6-diazaspiro[2.4]heptane-5,7-dione |
| diazaspiro[2.4]heptane-5,7-dione | |
| 4-(2,2′-bipyridin)-4-ylmethyl-6-(4- | 4-(2-hydroxymethylpyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(2-chloropyridin)-4-ylmethyl-6-(4- | 4-(2-fluoropyridin)-4-ylmethyl-6-(4-trifluoromethoxy- |
| trifluoromethoxyphenyl)-4,6- | phenyl)-4,6-diazaspiro[2.4]heptane-5,7-dione |
| diazaspiro[2.4]heptane-5,7-dione | |
| 4-(2-bromopyridin)-4-ylmethyl-6-(4- | 4-(2-aminopyridin)-4-ylmethyl-6-(4-trifluoromethoxy- |
| trifluoromethoxyphenyl)-4,6- | phenyl)-4,6-diazaspiro[2.4]heptane-5,7-dione |
| diazaspiro[2.4]heptane-5,7-dione | |
| 4-(2-methylaminopyridin)-4-ylmethyl-6-(4-trifluoro- | 4-(2-ethylaminopyridin)-4-ylmethyl-6-(4-trifluoro- |
| methoxyphenyl)-4,6-diazaspiro[2.4]heptane-5,7- | methoxyphenyl)-4,6-diazaspiro[2.4]heptane-5,7-dione |
| dione | |
| 4-(2-isopropylaminopyridin)-4-ylmethyl-6-(4- | 4-(2-phenylaminopyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(2-cyclohexylaminopyridin)-4-ylmethyl-6-(4- | 4-(2-diethylaminopyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(2-N-cyclohexyl-N-methylaminopyridin)-4- | 4-(2-piperidinopyridin)-4-ylmethyl-6-(4-trifluoro- |
| ylmethyl-6-(4-trifluoromethoxyphenyl)-4,6- | methoxyphenyl)-4,6-diazaspiro[2.4]heptane-5,7-dione |
| diazaspiro[2.4]heptane-5,7-dione | |
| 4-(2-morpholinopyridin)-4-ylmethyl-6-(4-trifluoro- | 4-[2-(4-methylpiperazino)pyridin]-4-ylmethyl-6-(4- |
| methoxyphenyl)-4,6-diazaspiro[2.4]heptane-5,7- | trifluoromethoxyphenyl)-4,6-diazaspiro[2.4]heptane- |
| dione | 5,7-dione |
| 4-[2-(1H-pyrazol-1-yl)pyridin]-4-ylmethyl-6-(4- | 4-[2-(2-benzoylhydrazino)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-benzoylaminopyridin)-4-ylmethyl-6-(4- | 4-[2-(4-chlorobenzoylamino)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(3-chlorobenzoylamino)pyridin]-4-ylmethyl-6- | 4-[2-(2-chlorobenzoylamino)pyridin]-4-ylmethyl-6-(4- |
| (4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(4-fluorobenzoylamino)pyridin]-4-ylmethyl-6- | 4-[2-(4-methoxybenzoylamino)pyridin]-4-ylmethyl-6- |
| (4-trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(2-acetamidopyridin)-4-ylmethyl-6-(4- | 4-(2-phenylacetamidopyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(4-methylpiperazino)acetamidopyridin]-4- | 4-(2-cyclohexanylcarboxamidopyridin)-4-ylmethyl-6- |
| ylmethyl-6-(4-trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-[2-(1-methyl-4- | 4-[2-(2-furanylcarbonyl)aminopyridin]-4-ylmethyl-6- |
| piperidinyl)carbonylaminopyridin]-4-ylmethyl-6-(4- | (4-trifluoromethoxyphenyl)-4,6-diazaspiro[2.4]heptane- |
| trifluoromethoxyphenyl)-4,6- | 5,7-dione |
| diazaspiro[2.4]heptane-5,7-dione | |
| 4-(2-ethoxycarbonylaminopyridin)-4-ylmethyl-6-(4- | 4-(2-isopropoxycarbonylaminopyridin)-4-ylmethyl-6- |
| trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(2-butoxycarbonylaminopyridin)-4-ylmethyl-6-(4- | 4-[2-(2-diethylaminoethoxycarbonylamino)pyridin]-4- |
| trifluoromethoxyphenyl)-4,6- | ylmethyl-6-(4-trifluoromethoxyphenyl)-4,6- |
| diazaspiro[2.4]heptane-5,7-dione | diazaspiro[2.4]heptane-5,7-dione |
| 4-(2-phenoxycarbonylaminopyridin)-4-ylmethyl-6- | 4-[2-(4-chloroanilino)carbonylaminopyridin]-4- |
| (4-trifluoromethoxyphenyl)-4,6- | ylmethyl)-6-(4-trifluoromethoxyphenyl)-4,6-diaza- |
| diazaspiro[2.4]heptane-5,7-dione | spiro[2.4]heptane-5,7-dione |
| 4-(2-anilinocarbonylaminopyridin)-4-ylmethyl-6-(4- | 4-(2-benzylaminocarbonylaminopyridin)-4-ylmethyl-6- |
| trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-[2-(2-pyridylamino)carbonylaminopyridin]-4- | 4-(2-ureidopyridin)-4-ylmethyl-6-(4-trifluoromethoxy- |
| ylmethyl-6-(4-trifluoromethoxyphenyl)-4,6- | phenyl)-4,6-diazaspiro[2.4]heptane-5,7-dione |
| diazaspiro[2.4]heptane-5,7-dione | |
| 4-(2-benzylaminosulfonylaminopyridin)-4-ylmethyl- | 4-(2-methylaminosulfonylaminopyridin)-4-ylmethyl-6- |
| 6-(4-trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(2-aminosulfonylaminopyridin)-4-ylmethyl-6-(4- | 4-(2-morpholinosulfonylaminopyridin)-4-ylmethyl-6- |
| trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(2-dimethylaminosulfonylaminopyridin)-4- | 4-(2-anilinosulfonylaminopyridin)-4-ylmethyl-6-(4- |
| ylmethyl-6-(4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-phenylsulfonylaminopyridin)-4-ylmethyl-6-(4- | 4-[2-(2-thienyl)sulfonylaminopyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-methylsulfonylaminopyridin)-4-ylmethyl-6-(4- | 4-[2-(4-chlorophenyl)sulfonylaminopyridin]-4- |
| trifluoromethoxyphenyl)-4,6- | ylmethyl-6-(4-trifluoromethoxyphenyl)-4,6- |
| diazaspiro[2.4]heptane-5,7-dione | diazaspiro[2.4]heptane-5,7-dione |
| 4-(2-benzylsulfonylaminopyridin)-4-ylmethyl-6-(4- | 4-(2-hydroxycarbonylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-ethoxycarbonylpyridin)-4-ylmethyl-6-(4- | 4-(2-tert-butoxycarbonylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-phenoxycarbonylpyridin)-4-ylmethyl-6-(4- | 4-[2-(4-methylpiperazinocarbonyl)pyridin]-4-ylmethyl- |
| trifluoromethoxyphenyl)-4,6- | 6-(4-trifluoromethoxyphenyl)-4,6- |
| diazaspiro[2.4]heptane-5,7-dione | diazaspiro[2.4]heptane-5,7-dione |
| 4-[2-(methylamino)carbonylpyridin]-4-ylmethyl-6- | 4-[2-(dimethylamino)carbonylpyridin]-4-ylmethyl-6-(4- |
| (4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-piperidinocarbonylpyridin)-4-ylmethyl-6-(4- | 4-(2-morpholinocarbonylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(4-phenylpiperazinocarbonyl)pyridin]-4- | 4-(2-phenylaminocarbonylpyridin)-4-ylmethyl-6-(4- |
| ylmethyl-6-(4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[2-(methylphenylamino)carbonylpyridin]-4- | 4-[2-(4-methoxyphenyl)methylaminocarbonylpyridin]- |
| ylmethyl-6-(4-trifluoromethoxyphenyl)-4,6- | 4-ylmethyl-6-(4-trifluoromethoxyphenyl)-4,6-diaza- |
| diazaspiro[2.4]heptane-5,7-dione | spiro[2.4]heptane-5,7-dione |
| 4-[2-(4-chlorophenyl)methylaminocarbonylpyridin]- | 4-[2-(4-morpholinylamino)carbonylpyridin]-4- |
| 4-ylmethyl-6-(4-trifluoromethoxyphenyl)-4,6-diaza- | ylmethyl-6-(4-trifluoromethoxyphenyl)-4,6- |
| spiro[2.4]heptane-5,7-dione | diazaspiro[2.4]heptane-5,7-dione |
| 4-(2-aminocarbonylpyridin)-4-ylmethyl-6-(4- | 4-(2-phenoxypyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-methoxypyridin)-4-ylmethyl-6-(4-trifluoro- | 4-(2-ethoxypyridin)-4-ylmethyl-6-(4-trifluoro- |
| methoxyphenyl)-4,6-diazaspiro[2.4]heptane-5,7- | methoxyphenyl)-4,6-diazaspiro[2.4]heptane-5,7-dione |
| dione | |
| 4-[2-(1-pyrrolidinyl)ethoxypyridin]-4-ylmethyl-6- | 4-(2-benzyloxypyridin)-4-ylmethyl-6-(4-trifluoro- |
| (4-trifluoromethoxyphenyl)-4,6- | methoxyphenyl)-4,6-diazaspiro[2.4]heptane-5,7-dione |
| diazaspiro[2.4]heptane-5,7-dione | |
| 4-(2-benzoyloxypyridin)-4-ylmethyl-6-(4-trifluoro- | 4-[2-(4,5-dihydro-1H-imidazol-2-yl)pyridin]-4- |
| methoxyphenyl)-4,6-diazaspiro[2.4]heptane-5,7- | ylmethyl-6-(4-trifluoromethoxyphenyl)-4,6- |
| dione | diazaspiro[2.4]heptane-5,7-dione |
| 4-[2-(1H-imidazol-2-yl)pyridin]-4-ylmethyl-6-(4- | 4-[2-(1H-tetrazol-5-yl)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(2-amidinopyridin)-4-ylmethyl-6-(4-trifluoro- | 4-(2-dimethylaminomethylpyridin)-4-ylmethyl-6-(4- |
| methoxyphenyl)-4,6-diazaspiro[2.4]heptane-5,7- | trifluoromethoxyphenyl)-4,6-diazaspiro[2.4]heptane- |
| dione | 5,7-dione |
| 4-(2-aminomethylpyridin)-4-ylmethyl-6-(4- | 4-(3-methylpyridin)-4-ylmethyl-6-(4-trifluoromethoxy- |
| trifluoromethoxyphenyl)-4,6- | phenyl)-4,6-diazaspiro[2.4]heptane-5,7-dione |
| diazaspiro[2.4]heptane-5,7-dione | |
| 4-(3-ethylpyridin)-4-ylmethyl-6-(4- | 4-(3-isopropylpyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(3-tert-butylpyridin)-4-ylmethyl-6-(4- | 4-(3-isobutylpyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(3-cyclohexylpyridin)-4-ylmethyl-6-(4-trifluoro- | 4-(3-phenylpyridin)-4-ylmethyl-6-(4-trifluoro- |
| methoxyphenyl)-4,6-diazaspiro[2.4]heptane-5,7- | methoxyphenyl)-4,6-diazaspiro[2.4]heptane-5,7-dione |
| dione | |
| 4-[3-(4-chlorophenyl)pyridin]-4-ylmethyl-6-(4- | 4-[3-(3-chlorophenyl)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(2-chlorophenyl)pyridin]-4-ylmethyl-6-(4- | 4-[3-(4-fluorophenyl)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(4-methoxyphenyl)pyridin]-4-ylmethyl-6-(4- | 4-(3-benzylpyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(3,2′-bipyridin)-4-ylmethyl-6-(4- | 4-(3-hydroxymethylpyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(3-chloropyridin)-4-ylmethyl-6-(4- | 4-(3-fluoropyridin)-4-ylmethyl-6-(4-trifluoromethoxy- |
| trifluoromethoxyphenyl)-4,6- | phenyl)-4,6-diazaspiro[2.4]heptane-5,7-dione |
| diazaspiro[2.4]heptane-5,7-dione | |
| 4-(3-bromopyridin)-4-ylmethyl-6-(4- | 4-(3-aminopyridin)-4-ylmethyl-6-(4-trifluoromethoxy- |
| trifluoromethoxyphenyl)-4,6- | phenyl)-4,6-diazaspiro[2.4]heptane-5,7-dione |
| diazaspiro[2.4]heptane-5,7-dione | |
| 4-(3-methylaminopyridin)-4-ylmethyl-6-(4- | 4-(3-ethylaminopyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(3-isopropylaminopyridin)-4-ylmethyl-6-(4- | 4-(3-phenylaminopyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(3-cyclohexylaminopyridin)-4-ylmethyl-6-(4- | 4-(3-diethylaminopyridin)-4-ylmethyl-6-(4-trifluoro- |
| trifluoromethoxyphenyl)-4,6- | methoxyphenyl)-4,6-diazaspiro[2.4]heptane-5,7-dione |
| diazaspiro[2.4]heptane-5,7-dione | |
| 4-(3-N-cyclohexyl-N-methylaminopyridin)-4- | 4-(3-piperidinopyridin)-4-ylmethyl-6-(4-trifluoro- |
| ylmethyl-6-(4-trifluoromethoxyphenyl)-4,6- | methoxyphenyl)-4,6-diazaspiro[2.4]heptane-5,7-dione |
| diazaspiro[2.4]heptane-5,7-dione | |
| 4-(3-morpholinopyridin)-4-ylmethyl-6-(4- | 4-[3-(4-methylpiperazino)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(1H-pyrazol-1-yl)pyridin]-4-ylmethyl-6-(4- | 4-[3-(2-benzoylhydrazino)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-benzoylaminopyridin)-4-ylmethyl-6-(4- | 4-[3-(4-chlorobenzoylamino)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(3-chlorobenzoylamino)pyridin]-4-ylmethyl-6- | 4-[3-(2-chlorobenzoylamino)pyridin]-4-ylmethyl-6-(4- |
| (4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(4-fluorobenzoylamino)pyridin]-4-ylmethyl-6- | 4-[3-(4-methoxybenzoylamino)pyridin]-4-ylmethyl-6- |
| (4-trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(3-acetamidopyridin)-4-ylmethyl-6-(4- | 4-(3-phenylacetamidopyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(4-methylpiperazino)acetamidopyridin]-4- | 4-(3-cyclohexanylcarboxamidopyridin)-4-ylmethyl-6- |
| ylmethyl-6-(4-trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-[3-(1-methyl-4- | 4-[3-(2-furanylcarbonyl)aminopyridin]-4-ylmethyl-6- |
| piperidinyl)carbonylaminopyridin]-4-ylmethyl-6-(4- | (4-trifluoromethoxyphenyl)-4,6-diazaspiro[2.4]heptane- |
| trifluoromethoxyphenyl)-4,6- | 5,7-dione |
| diazaspiro[2.4]heptane-5,7-dione | |
| 4-(3-ethoxycarbonylaminopyridin)-4-ylmethyl-6-(4- | 4-(3-isopropoxycarbonylaminopyridin)-4-ylmethyl-6- |
| trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(3-butoxycarbonylaminopyridin)-4-ylmethyl-6-(4- | 4-[3-(2-diethylaminoethoxycarbonylamino)pyridin]-4- |
| trifluoromethoxyphenyl)-4,6- | ylmethyl-6-(4-trifluoromethoxyphenyl)-4,6- |
| diazaspiro[2.4]heptane-5,7-dione | diazaspiro[2.4]heptane-5,7-dione |
| 4-(3-phenoxycarbonylaminopyridin)-4-ylmethyl-6- | 4-[3-(4-chloroanilino)carbonylaminopyridin]-4- |
| (4-trifluoromethoxyphenyl)-4,6- | ylmethyl)-6-(4-trifluoromethoxyphenyl)-4,6- |
| diazaspiro[2.4]heptane-5,7-dione | diazaspiro[2.4]heptane-5,7-dione |
| 4-(3-anilinocarbonylaminopyridin)-4-ylmethyl-6-(4- | 4-(3-benzylaminocarbonylaminopyridin)-4-ylmethyl-6- |
| trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-[3-(2-pyridylamino)carbonylaminopyridin]-4- | 4-(3-ureidopyridin)-4-ylmethyl-6-(4- |
| ylmethyl-6-(4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-benzylaminosulfonylaminopyridin)-4-ylmethyl- | 4-(3-methylaminosulfonylaminopyridin)-4-ylmethyl-6- |
| 6-(4-trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(3-aminosulfonylaminopyridin)-4-ylmethyl-6-(4- | 4-(3-morpholinosulfonylaminopyridin)-4-ylmethyl-6- |
| trifluoromethoxyphenyl)-4,6- | (4-trifluoromethoxyphenyl)-4,6-diazaspiro[2.4]heptane- |
| diazaspiro[2.4]heptane-5,7-dione | 5,7-dione |
| 4-(3-dimethylaminosulfonylaminopyridin)-4- | 4-(3-anilinosulfonylaminopyridin)-4-ylmethyl-6-(4- |
| ylmethyl-6-(4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-phenylsulfonylaminopyridin)-4-ylmethyl-6-(4- | 4-[3-(2-thienyl)sulfonylaminopyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-methylsulfonylaminopyridin)-4-ylmethyl-6-(4- | 4-[3-(4-chlorophenyl)sulfonylaminopyridin]-4- |
| trifluoromethoxyphenyl)-4,6- | ylmethyl-6-(4-trifluoromethoxyphenyl)-4,6- |
| diazaspiro[2.4]heptane-5,7-dione | diazaspiro[2.4]heptane-5,7-dione |
| 4-(3-benzylsulfonylaminopyridin)-4-ylmethyl-6-(4- | 4-(3-hydroxycarbonylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-ethoxycarbonylpyridin)-4-ylmethyl-6-(4- | 4-(3-tert-butoxycarbonylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-phenoxycarbonylpyridin)-4-ylmethyl-6-(4- | 4-[3-(4-methylpiperazinocarbonyl)pyridin]-4-ylmethyl- |
| trifluoromethoxyphenyl)-4,6- | 6-(4-trifluoromethoxyphenyl)-4,6- |
| diazaspiro[2.4]heptane-5,7-dione | diazaspiro[2.4]heptane-5,7-dione |
| 4-[3-(methylamino)carbonylpyridin]-4-ylmethyl-6- | 4-[3-(dimethylamino)carbonylpyridin]-4-ylmethyl-6-(4- |
| (4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-piperidinocarbonylpyridin)-4-ylmethyl-6-(4- | 4-(3-morpholinocarbonylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(4-phenylpiperazinocarbonyl)pyridin]-4- | 4-(3-phenylaminocarbonylpyridin)-4-ylmethyl-6-(4- |
| ylmethyl-6-(4-trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-[3-(methylphenylamino)carbonylpyridin]-4- | 4-[3-(4-methoxyphenyl)methylaminocarbonylpyridin]- |
| ylmethyl-6-(4-trifluoromethoxyphenyl)-4,6- | 4-ylmethyl-6-(4-trifluoromethoxyphenyl)-4,6- |
| diazaspiro[2.4]heptane-5,7-dione | diazaspiro[2.4]heptane-5,7-dione |
| 4-[3-(4-chlorophenyl)methylaminocarbonylpyridin]- | 4-[3-(4-morpholinylamino)carbonylpyridin]-4- |
| 4-ylmethyl-6-(4-trifluoromethoxyphenyl)-4,6- | ylmethyl-6-(4-trifluoromethoxyphenyl)-4,6- |
| diazaspiro[2.4]heptane-5,7-dione | diazaspiro[2.4]heptane-5,7-dione |
| 4-(3-aminocarbonylpyridin)-4-ylmethyl-6-(4- | 4-(3-phenoxypyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-methoxypyridin)-4-ylmethyl-6-(4- | 4-(3-ethoxypyridin)-4-ylmethyl-6-(4-trifluoromethoxy- |
| trifluoromethoxyphenyl)-4,6- | phenyl)-4,6-diazaspiro[2.4]heptane-5,7-dione |
| diazaspiro[2.4]heptane-5,7-dione | |
| 4-[3-(1-pyrrolidinyl)ethoxypyridin]-4-ylmethyl-6- | 4-(3-benzyloxypyridin)-4-ylmethyl-6-(4-trifluoro- |
| (4-trifluoromethoxyphenyl)-4,6- | methoxyphenyl)-4,6-diazaspiro[2.4]heptane-5,7-dione |
| diazaspiro[2.4]heptane-5,7-dione | |
| 4-(3-benzoyloxypyridin)-4-ylmethyl-6-(4-trifluoro- | 4-[3-(4,5-dihydro-1H-imidazol-2-yl)pyridin]-4- |
| methoxyphenyl)-4,6-diazaspiro[2.4]heptane-5,7- | ylmethyl-6-(4-trifluoromethoxyphenyl)-4,6- |
| dione | diazaspiro[2.4]heptane-5,7-dione |
| 4-[3-(1H-imidazol-2-yl)pyridin]-4-ylmethyl-6-(4- | 4-[3-(1H-tetrazol-5-yl)pyridin]-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-amidinopyridin)-4-ylmethyl-6-(4- | 4-(3-dimethylaminomethylpyridin)-4-ylmethyl-6-(4- |
| trifluoromethoxyphenyl)-4,6- | trifluoromethoxyphenyl)-4,6-diazaspiro[2.4]heptane- |
| diazaspiro[2.4]heptane-5,7-dione | 5,7-dione |
| 4-(3-aminomethylpyridin)-4-ylmethyl-6-(4-trifluoro- | 5-methyl-1-(2-methylpyrid-4-ylmethyl)-3-(4-trifluoro- |
| methoxyphenyl)-4,6-diazaspiro[2.4]heptane-5,7- | methoxyphenyl)imidazolidine-2,4-dione |
| dione | |
| 5-methyl-1-(2-ethylpyrid-4-ylmethyl)-3-(4-trifluoro- | 5-methyl-1-(2-isopropylpyrid-4-ylmethyl)-3-(4- |
| methoxyphenyl)imidazolidine-2,4-dione | trifluoromethoxyphenyl)imidazolidine-2,4-dione |
| 5-methyl-1-(2-tert-butylpyrid-4-ylmethyl)-3-(4- | 5-methyl-1-(2-isobutylpyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-(2-cyclohexylpyrid-4-ylmethyl)-3-(4- | 5-methyl-1-(2-phenylpyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-[2-(4-chlorophenyl)pyrid-4-ylmethyl]-3- | 5-methyl-1-[2-(3-chlorophenyl)pyrid-4-ylmethyl]-3-(4- |
| (4-trifluoromethoxyphenyl)imidazolidine-2,4-dione | trifluoromethoxyphenyl)imidazolidine-2,4-dione |
| 5-methyl-1-[2-(2-chlorophenyl)pyrid-4-ylmethyl]-3- | 5-methyl-1-[2-(4-fluorophenyl)pyrid-4-ylmethyl]-3-(4- |
| (4-trifluoromethoxyphenyl)imidazolidine-2,4-dione | trifluoromethoxyphenyl)imidazolidine-2,4-dione |
| 5-methyl-1-[2-(4-methoxyphenyl)pyrid-4-ylmethyl]- | 5-methyl-1-(2-benzylpyrid-4-ylmethyl)-3-(4-trifluoro- |
| 3-(4-trifluoromethoxyphenyl)imidazolidine-2,4- | methoxyphenyl)imidazolidine-2,4-dione |
| dione | |
| 5-methyl-1-(2,2′-bipyrid-4-ylmethyl)-3-(4- | 5-methyl-1-(2-hydroxymethylpyrid-4-ylmethyl)-3-(4- |
| trifluoromethoxyphenyl)imidazolidine-2,4-dione | trifluoromethoxyphenyl)imidazolidine-2,4-dione |
| 5-methyl-1-(2-chloropyrid-4-ylmethyl)-3-(4- | 5-methyl-1-(2-fluoropyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-(2-bromopyrid-4-ylmethyl)-3-(4- | 5-methyl-1-(2-aminopyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-(2-methylaminopyrid-4-ylmethyl)-3-(4- | 5-methyl-1-(2-ethylaminopyrid-4-ylmethyl)-3-(4- |
| trifluoromethoxyphenyl)imidazolidine-2,4-dione | trifluoromethoxyphenyl)imidazolidine-2,4-dione |
| 5-methyl-1-(2-isopropylaminopyrid-4-ylmethyl)-3- | 5-methyl-1-(2-phenylaminopyrid-4-ylmethyl)-3-(4- |
| (4-trifluoromethoxyphenyl)imidazolidine-2,4-dione | trifluoromethoxyphenyl)imidazolidine-2,4-dione |
| 5-methyl-1-(2-cyclohexylaminopyrid-4-ylmethyl)-3- | 5-methyl-1-(2-diethylaminopyrid-4-ylmethyl)-3-(4- |
| (4-trifluoromethoxyphenyl)imidazolidine-2,4-dione | trifluoromethoxyphenyl)imidazolidine-2,4-dione |
| 5-methyl-1-(2-N-cyclohexyl-N-methylaminopyrid- | 5-methyl-1-(2-piperidinopyrid-4-ylmethyl)-3-(4- |
| 4-ylmethyl)-3-(4- | trifluoromethoxyphenyl)imidazolidine-2,4-dione |
| trifluoromethoxyphenyl)imidazolidine-2,4-dione | |
| 5-methyl-1-(2-morpholinopyrid-4-ylmethyl)-3-(4- | 5-methyl-1-[2-(4-methylpiperazino)pyrid-4-ylmethyl]- |
| trifluoromethoxyphenyl)imidazolidine-2,4-dione | 3-(4-trifluoromethoxyphenyl)imidazolidine-2,4-dione |
| 5-methyl-1-[2-(1H-pyrazol-1-yl)pyrid-4-ylmethyl]- | 5-methyl-1-[2-(2-benzoylhydrazino)pyrid-4-ylmethyl]- |
| 3-(4-trifluoromethoxyphenyl)imidazolidine-2,4- | 3-(4-trifluoromethoxyphenyl)imidazolidine-2,4-dione |
| dione | |
| 5-methyl-1-(2-benzoylaminopyrid-4-ylmethyl)-3-(4- | 5-methyl-1-[2-(4-chlorobenzoylamino)pyrid-4- |
| trifluoromethoxyphenyl)imidazolidine-2,4-dione | ylmethyl]-3-(4-trifluoromethoxyphenyl)imidazolidine- |
| | 2,4-dione |
| 5-methyl-1-[2-(3-chlorobenzoylamino)pyrid-4- | 5-methyl-1-[2-(2-chlorobenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-[2-(4-fluorobenzoylamino)pyrid-4- | 5-methyl-1-[2-(4-methoxybenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(2-acetamidopyrid-4-ylmethyl)-3-(4- | 5-methyl-1-(2-phenylacetamidopyrid-4-ylmethyl)-3-(4- |
| trifluoromethoxyphenyl)imidazolidine-2,4-dione | trifluoromethoxyphenyl)imidazolidine-2,4-dione |
| 5-methyl-1-[2-(4-methylpiperazino)acetamidopyrid- | 5-methyl-1-(2-cyclohexanylcarboxamidopyrid-4- |
| 4-ylmethyl]-3-(4- | ylmethyl)-3-(4-trifluoromethoxyphenyl)imidazolidine- |
| trifluoromethoxyphenyl)imidazolidine-2,4-dione | 2,4-dione |
| 5-methyl-1-[2-(1-methyl-4- | 5-methyl-1-[2-(2-furanylcarbonyl)aminopyrid-4-yl- |
| piperidinyl)carbonylaminopyrid-4-ylmethyl]-3-(4- | methyl]-3-(4-trifluoromethoxyphenyl)imidazolidine- |
| trifluoromethoxyphenyl)imidazolidine-2,4-dione | 2,4-dione |
| 5-methyl-1-(2-ethoxycarbonylaminopyrid-4- | 5-methyl-1-(2-isopropoxycarbonylaminopyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5-methyl-1-(2-butoxycarbonylaminopyrid-4- | 5-methyl-1-[2-(2- |
| ylmethyl)-3-(4- | diethylaminoethoxycarbonylamino)pyrid-4-ylmethyl]- |
| trifluoromethoxyphenyl)imidazolidine-2,4-dione | 3-(4-trifluoromethoxyphenyl)imidazolidine-2,4-dione |
| 5-methyl-1-(2-phenoxycarbonylaminopyrid-4- | 5-methyl-1-[2-(4-chloroanilino)carbonylaminopyrid-4- |
| ylmethyl)-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(2-anilinocarbonylaminopyrid-4- | 5-methyl-1-(2-benzylaminocarbonylaminopyrid-4-yl- |
| ylmethyl)-3-(4- | methyl)-3-(4-trifluoromethoxyphenyl)imidazolidine- |
| trifluoromethoxyphenyl)imidazolidine-2,4-dione | 2,4-dione |
| 5-methyl-1-[2-(2- | 5-methyl-1-(2-ureidopyrid-4-ylmethyl)-3-(4-trifluoro- |
| pyridylamino)carbonylaminopyrid-4-ylmethyl]-3-(4- | methoxyphenyl)imidazolidine-2,4-dione |
| trifluoromethoxyphenyl)imidazolidine-2,4-dione | |
| 5-methyl-1-(2-benzylaminosulfonylaminopyrid-4- | 5-methyl-1-(2-methylaminosulfonylaminopyrid-4-yl- |
| ylmethyl)-3-(4- | methyl)-3-(4-trifluoromethoxyphenyl)imidazolidine- |
| trifluoromethoxyphenyl)imidazolidine-2,4-dione | 2,4-dione |
| 5-methyl-1-(2-aminosulfonylaminopyrid-4- | 5-methyl-1-(2-morpholinosulfonylaminopyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5-methyl-1-(2-dimethylaminosulfonylaminopyrid-4- | 5-methyl-1-(2-anilinosulfonylaminopyrid-4-ylmethyl)- |
| ylmethyl)-3-(4- | 3-(4-trifluoromethoxyphenyl)imidazolidine-2,4-dione |
| trifluoromethoxyphenyl)imidazolidine-2,4-dione | |
| 5-methyl-1-(2-phenylsulfonylaminopyrid-4- | 5-methyl-1-[2-(2-thienyl)sulfonylaminopyrid-4-yl- |
| ylmethyl)-3-(4- | methyl]-3-(4-trifluoromethoxyphenyl)imidazolidine- |
| trifluoromethoxyphenyl)imidazolidine-2,4-dione | 2,4-dione |
| 5-methyl-1-(2-methylsulfonylaminopyrid-4- | 5-methyl-1-[2-(4-chlorophenyl)sulfonylaminopyrid-4- |
| ylmethyl)-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(2-benzylsulfonylaminopyrid-4- | 5-methyl-1-(2-hydroxycarbonylpyrid-4-ylmethyl)-3-(4- |
| ylmethyl)-3-(4- | trifluoromethoxyphenyl)imidazolidine-2,4-dione |
| trifluoromethoxyphenyl)imidazolidine-2,4-dione | |
| 5-methyl-1-(2-ethoxycarbonylpyrid-4-ylmethyl)-3- | 5-methyl-1-(2-tert-butoxycarbonylpyrid-4-ylmethyl)-3- |
| (4-trifluoromethoxyphenyl)imidazolidine-2,4-dione | (4-trifluoromethoxyphenyl)imidazolidine-2,4-dione |
| 5-methyl-1-(2-phenoxycarbonylpyrid-4-ylmethyl)-3- | 5-methyl-1-[2-(4-methylpiperazinocarbonyl)pyrid-4-yl- |
| (4-trifluoromethoxyphenyl)imidazolidine-2,4-dione | methyl]-3-(4-trifluoromethoxyphenyl)imidazolidine- |
| | 2,4-dione |
| 5-methyl-1-[2-(methylamino)carbonylpyrid-4- | 5-methyl-1-[2-(dimethylamino)carbonylpyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(2-piperidinocarbonylpyrid-4-ylmethyl)- | 5-methyl-1-(2-morpholinocarbonylpyrid-4-ylmethyl)-3- |
| 3-(4-trifluoromethoxyphenyl)imidazolidine-2,4- | (4-trifluoromethoxyphenyl)imidazolidine-2,4-dione |
| dione | |
| 5-methyl-1-[2-(4-phenylpiperazinocarbonyl)pyrid-4- | 5-methyl-1-(2-phenylaminocarbonylpyrid-4-ylmethyl)- |
| ylmethyl]-3-(4- | 3-(4-trifluoromethoxyphenyl)imidazolidine-2,4-dione |
| trifluoromethoxyphenyl)imidazolidine-2,4-dione | |
| 5-methyl-1-[2-(methylphenylamino)carbonylpyrid- | 5-methyl-1-[2-(4- |
| 4-ylmethyl]-3-(4- | methoxyphenyl)methylaminocarbonylpyrid-4- |
| trifluoromethoxyphenyl)imidazolidine-2,4-dione | ylmethyl]-3-(4-trifluoromethoxyphenyl)imidazolidine- |
| | 2,4-dione |
| 5-methyl-1-[2-(4- | 5-methyl-1-[2-(4-morpholinylamino)carbonylpyrid-4- |
| chlorophenyl)methylaminocarbonylpyrid-4- | ylmethyl]-3-(4-trifluoromethoxyphenyl)imidazolidine- |
| ylmethyl]-3-(4- | 2,4-dione |
| trifluoromethoxyphenyl)imidazolidine-2,4-dione | |
| 5-methyl-1-(2-aminocarbonylpyrid-4-ylmethyl)-3- | 5-methyl-1-(2-phenoxypyrid-4-ylmethyl)-3-(4- |
| (4-trifluoromethoxyphenyl)imidazolidine-2,4-dione | trifluoromethoxyphenyl)imidazolidine-2,4-dione |
| 5-methyl-1-(2-methoxypyrid-4-ylmethyl)-3-(4- | 5-methyl-1-(2-ethoxypyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-[2-(1-pyrrolidinyl)ethoxypyrid-4- | 5-methyl-1-(2-benzyloxypyrid-4-ylmethyl)-3-(4- |
| ylmethyl]-3-(4- | trifluoromethoxyphenyl)imidazolidine-2,4-dione |
| trifluoromethoxyphenyl)imidazolidine-2,4-dione | |
| 5-methyl-1-(2-benzoyloxypyrid-4-ylmethyl)-3-(4- | 5-methyl-1-[2-(4,5-dihydro-1H-imidazol-2-yl)pyrid-4- |
| trifluoromethoxyphenyl)imidazolidine-2,4-dione | ylmethyl]-3-(4-trifluoromethoxyphenyl)imidazolidine- |
| | 2,4-dione |
| 5-methyl-1-[2-(1H-imidazol-2-yl)pyrid-4-ylmethyl]- | 5-methyl-1-[2-(1H-tetrazol-5-yl)pyrid-4-ylmethyl]-3- |
| 3-(4-trifluoromethoxyphenyl)imidazolidine-2,4- | (4-trifluoromethoxyphenyl)imidazolidine-2,4-dione |
| dione | |
| 5-methyl-1-(2-amidinopyrid-4-ylmethyl)-3-(4- | 5-methyl-1-(2-dimethylaminomethylpyrid-4-ylmethyl)- |
| trifluoromethoxyphenyl)imidazolidine-2,4-dione | 3-(4-trifluoromethoxyphenyl)imidazolidine-2,4-dione |
| 5-methyl-1-(2-aminomethylpyrid-4-ylmethyl)-3-(4- | 5-methyl-1-(3-methylpyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-(3-ethylpyrid-4-ylmethyl)-3-(4-trifluoro- | 5-methyl-1-(3-isopropylpyrid-4-ylmethyl)-3-(4- |
| methoxyphenyl)imidazolidine-2,4-dione | trifluoromethoxyphenyl)imidazolidine-2,4-dione |
| 5-methyl-1-(3-tert-butylpyrid-4-ylmethyl)-3-(4- | 5-methyl-1-(3-isobutylpyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-(3-cyclohexylpyrid-4-ylmethyl)-3-(4- | 5-methyl-1-(3-phenylpyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-[3-(4-chlorophenyl)pyrid-4-ylmethyl]-3- | 5-methyl-1-[3-(3-chlorophenyl)pyrid-4-ylmethyl]-3-(4- |
| (4-trifluoromethoxyphenyl)imidazolidine-2,4-dione | trifluoromethoxyphenyl)imidazolidine-2,4-dione |
| 5-methyl-1-[3-(2-chlorophenyl)pyrid-4-ylmethyl]-3- | 5-methyl-1-[3-(4-fluorophenyl)pyrid-4-ylmethyl]-3-(4- |
| (4-trifluoromethoxyphenyl)imidazolidine-2,4-dione | trifluoromethoxyphenyl)imidazolidine-2,4-dione |
| 5-methyl-1-[3-(4-methoxyphenyl)pyrid-4-ylmethyl]- | 5-methyl-1-(3-benzylpyrid-4-ylmethyl)-3-(4-trifluoro- |
| 3-(4-trifluoromethoxyphenyl)imidazolidine-2,4- | methoxyphenyl)imidazolidine-2,4-dione |
| dione | |
| 5-methyl-1-(3,2′-bipyrid-4-ylmethyl)-3-(4- | 5-methyl-1-(3-hydroxymethylpyrid-4-ylmethyl)-3-(4- |
| trifluoromethoxyphenyl)imidazolidine-2,4-dione | trifluoromethoxyphenyl)imidazolidine-2,4-dione |
| 5-methyl-1-(3-chloropyrid-4-ylmethyl)-3-(4- | 5-methyl-1-(3-fluoropyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-(3-bromopyrid-4-ylmethyl)-3-(4- | 5-methyl-1-(3-aminopyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-(3-methylaminopyrid-4-ylmethyl)-3-(4- | 5-methyl-1-(3-ethylaminopyrid-4-ylmethyl)-3-(4- |
| trifluoromethoxyphenyl)imidazolidine-2,4-dione | trifluoromethoxyphenyl)imidazolidine-2,4-dione |
| 5-methyl-1-(3-isopropylaminopyrid-4-ylmethyl)-3- | 5-methyl-1-(3-phenylaminopyrid-4-ylmethyl)-3-(4- |
| (4-trifluoromethoxyphenyl)imidazolidine-2,4-dione | trifluoromethoxyphenyl)imidazolidine-2,4-dione |
| 5-methyl-1-(3-cyclohexylaminopyrid-4-ylmethyl)-3- | 5-methyl-1-(3-diethylaminopyrid-4-ylmethyl)-3-(4- |
| (4-trifluoromethoxyphenyl)imidazolidine-2,4-dione | trifluoromethoxyphenyl)imidazolidine-2,4-dione |
| 5-methyl-1-(3-N-cyclohexyl-N-methylaminopyrid- | 5-methyl-1-(3-piperidinopyrid-4-ylmethyl)-3-(4- |
| 4-ylmethyl)-3-(4- | trifluoromethoxyphenyl)imidazolidine-2,4-dione |
| trifluoromethoxyphenyl)imidazolidine-2,4-dione | |
| 5-methyl-1-(3-morpholinopyrid-4-ylmethyl)-3-(4- | 5-methyl-1-[3-(4-methylpiperazino)pyrid-4-ylmethyl]- |
| trifluoromethoxyphenyl)imidazolidine-2,4-dione | 3-(4-trifluoromethoxyphenyl)imidazolidine-2,4-dione |
| 5-methyl-1-[3-(1H-pyrazol-1-yl)pyrid-4-ylmethyl]- | 5-methyl-1-[3-(2-benzoylhydrazino)pyrid-4-ylmethyl]- |
| 3-(4-trifluoromethoxyphenyl)imidazolidine-2,4- | 3-(4-trifluoromethoxyphenyl)imidazolidine-2,4-dione |
| dione | |
| 5-methyl-1-(3-benzoylaminopyrid-4-ylmethyl)-3-(4- | 5-methyl-1-[3-(4-chlorobenzoylamino)pyrid-4- |
| trifluoromethoxyphenyl)imidazolidine-2,4-dione | ylmethyl]-3-(4-trifluoromethoxyphenyl)imidazolidine- |
| | 2,4-dione |
| 5-methyl-1-[3-(3-chlorobenzoylamino)pyrid-4- | 5-methyl-1-[3-(2-chlorobenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-[3-(4-fluorobenzoylamino)pyrid-4- | 5-methyl-1-[3-(4-methoxybenzoylamino)pyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(3-acetamidopyrid-4-ylmethyl)-3-(4- | 5-methyl-1-(3-phenylacetamidopyrid-4-ylmethyl)-3-(4- |
| trifluoromethoxyphenyl)imidazolidine-2,4-dione | trifluoromethoxyphenyl)imidazolidine-2,4-dione |
| 5-methyl-1-[3-(4-methylpiperazino)acetamidopyrid- | 5-methyl-1-(3-cyclohexanylcarboxamidopyrid-4- |
| 4-ylmethyl]-3-(4- | ylmethyl)-3-(4-trifluoromethoxyphenyl)imidazolidine- |
| trifluoromethoxyphenyl)imidazolidine-2,4-dione | 2,4-dione |
| 5-methyl-1-[3-(1-methyl-4- | 5-methyl-1-[3-(2-furanylcarbonyl)aminopyrid-4-yl- |
| piperidinyl)carbonylaminopyrid-4-ylmethyl]-3-(4- | methyl]-3-(4-trifluoromethoxyphenyl)imidazolidine- |
| trifluoromethoxyphenyl)imidazolidine-2,4-dione | 2,4-dione |
| 5-methyl-1-(3-ethoxycarbonylaminopyrid-4- | 5-methyl-1-(3-isopropoxycarbonylaminopyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5-methyl-1-(3-butoxycarbonylaminopyrid-4- | 5-methyl-1-[3-(2- |
| ylmethyl)-3-(4- | diethylaminoethoxycarbonylamino)pyrid-4-ylmethyl]- |
| trifluoromethoxyphenyl)imidazolidine-2,4-dione | 3-(4-trifluoromethoxyphenyl)imidazolidine-2,4-dione |
| 5-methyl-1-(3-phenoxycarbonylaminopyrid-4- | 5-methyl-1-[3-(4-chloroanilino)carbonylaminopyrid-4- |
| ylmethyl)-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(3-anilinocarbonylaminopyrid-4- | 5-methyl-1-(3-benzylaminocarbonylaminopyrid-4-yl- |
| ylmethyl)-3-(4- | methyl)-3-(4-trifluoromethoxyphenyl)imidazolidine- |
| trifluoromethoxyphenyl)imidazolidine-2,4-dione | 2,4-dione |
| 5-methyl-1-[3-(2- | 5-methyl-1-(3-ureidopyrid-4-ylmethyl)-3-(4-trifluoro- |
| pyridylamino)carbonylaminopyrid-4-ylmethyl]-3-(4- | methoxyphenyl)imidazolidine-2,4-dione |
| trifluoromethoxyphenyl)imidazolidine-2,4-dione | |
| 5-methyl-1-(3-benzylaminosulfonylaminopyrid-4- | 5-methyl-1-(3-methylaminosulfonylaminopyrid-4-yl- |
| ylmethyl)-3-(4- | methyl)-3-(4-trifluoromethoxyphenyl)imidazolidine- |
| trifluoromethoxyphenyl)imidazolidine-2,4-dione | 2,4-dione |
| 5-methyl-1-(3-aminosulfonylaminopyrid-4- | 5-methyl-1-(3-morpholinosulfonylaminopyrid-4- |
| ylmethyl)-3-(4- | ylmethyl)-3-(4-trifluoromethoxyphenyl)imidazolidine- |
| trifluoromethoxyphenyl)imidazolidine-2,4-dione | 2,4-dione |
| 5-methyl-1-(3-dimethylaminosulfonylaminopyrid-4- | 5-methyl-1-(3-anilinosulfonylaminopyrid-4-ylmethyl)- |
| ylmethyl)-3-(4- | 3-(4-trifluoromethoxyphenyl)imidazolidine-2,4-dione |
| trifluoromethoxyphenyl)imidazolidine-2,4-dione | |
| 5-methyl-1-(3-phenylsulfonylaminopyrid-4- | 5-methyl-1-[3-(2-thienyl)sulfonylaminopyrid-4- |
| ylmethyl)-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(3-methylsulfonylaminopyrid-4- | 5-methyl-1-[3-(4-chlorophenyl)sulfonylaminopyrid-4- |
| ylmethyl)-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(3-benzylsulfonylaminopyrid-4- | 5-methyl-1-(3-hydroxycarbonylpyrid-4-ylmethyl)-3-(4- |
| ylmethyl)-3-(4- | trifluoromethoxyphenyl)imidazolidine-2,4-dione |
| trifluoromethoxyphenyl)imidazolidine-2,4-dione | |
| 5-methyl-1-(3-ethoxycarbonylpyrid-4-ylmethyl)-3- | 5-methyl-1-(3-tert-butoxycarbonylpyrid-4-ylmethyl)-3- |
| (4-trifluoromethoxyphenyl)imidazolidine-2,4-dione | (4-trifluoromethoxyphenyl)imidazolidine-2,4-dione |
| 5-methyl-1-(3-phenoxycarbonylpyrid-4-ylmethyl)-3- | 5-methyl-1-[3-(4-methylpiperazinocarbonyl)pyrid-4-yl- |
| (4-trifluoromethoxyphenyl)imidazolidine-2,4-dione | methyl]-3-(4-trifluoromethoxyphenyl)imidazolidine- |
| | 2,4-dione |
| 5-methyl-1-[3-(methylamino)carbonylpyrid-4- | 5-methyl-1-[3-(dimethylamino)carbonylpyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 5-methyl-1-(3-piperidinocarbonylpyrid-4-ylmethyl)- | 5-methyl-1-(3-morpholinocarbonylpyrid-4-ylmethyl)-3- |
| 3-(4-trifluoromethoxyphenyl)imidazolidine-2,4- | (4-trifluoromethoxyphenyl)imidazolidine-2,4-dione |
| dione | |
| 5-methyl-1-[3-(4-phenylpiperazinocarbonyl)pyrid-4- | 5-methyl-1-(3-phenylaminocarbonylpyrid-4-ylmethyl)- |
| ylmethyl]-3-(4- | 3-(4-trifluoromethoxyphenyl)imidazolidine-2,4-dione |
| trifluoromethoxyphenyl)imidazolidine-2,4-dione | |
| 5-methyl-1-[3-(methylphenylamino)carbonylpyrid- | 5-methyl-1-[3-(4- |
| 4-ylmethyl]-3-(4- | methoxyphenyl)methylaminocarbonylpyrid-4- |
| trifluoromethoxyphenyl)imidazolidine-2,4-dione | ylmethyl]-3-(4-trifluoromethoxyphenyl)imidazolidine- |
| | 2,4-dione |
| 5-methyl-1-[3-(4- | 5-methyl-1-[3-(4-morpholinylamino)carbonylpyrid-4- |
| chlorophenyl)methylaminocarbonyrpyrid-4- | ylmethyl]-3-(4-trifluoromethoxyphenyl)imidazolidine- |
| ylmethyl]-3-(4- | 2,4-dione |
| trifluoromethoxyphenyl)imidazolidine-2,4-dione | |
| 5-methyl-1-(3-aminocarbonylpyrid-4-ylmethyl)-3- | 5-methyl-1-(3-phenoxypyrid-4-ylmethyl)-3-(4- |
| (4-trifluoromethoxyphenyl)imidazolidine-2,4-dione | trifluoromethoxyphenyl)imidazolidine-2,4-dione |
| 5-methyl-1-(3-methoxypyrid-4-ylmethyl)-3-(4- | 5-methyl-1-(3-ethoxypyrid-4-ylmethyl)-3-(4-trifluoro- |
| trifluoromethoxyphenyl)imidazolidine-2,4-dione | methoxyphenyl)imidazolidine-2,4-dione |
| 5-methyl-1-[3-(1-pyrrolidinyl)ethoxypyrid-4- | 5-methyl-1-(3-benzyloxypyrid-4-ylmethyl)-3-(4- |
| ylmethyl]-3-(4- | trifluoromethoxyphenyl)imidazolidine-2,4-dione |
| trifluoromethoxyphenyl)imidazolidine-2,4-dione | |
| 5-methyl-1-(3-benzoyloxypyrid-4-ylmethyl)-3-(4- | 5-methyl-1-[3-(4,5-dihydro-1H-imidazol-2-yl)pyrid-4- |
| trifluoromethoxyphenyl)imidazolidine-2,4-dione | ylmethyl]-3-(4-trifluoromethoxyphenyl)imidazolidine- |
| | 2,4-dione |
| 5-methyl-1-[3-(1H-imidazol-2-yl)pyrid-4-ylmethyl]- | 5-methyl-1-[3-(1H-tetrazol-5-yl)pyrid-4-ylmethyl]-3- |
| 3-(4-trifluoromethoxyphenyl)imidazolidine-2,4- | (4-trifluoromethoxyphenyl)imidazolidine-2,4-dione |
| dione | |
| 5-methyl-1-(3-amidinopyrid-4-ylmethyl)-3-(4- | 5-methyl-1-(3-dimethylaminomethylpyrid-4-ylmethyl)- |
| trifluoromethoxyphenyl)imidazolidine-2,4-dione | 3-(4-trifluoromethoxyphenyl)imidazolidine-2,4-dione |
| 5-methyl-1-(3-aminomethylpyrid-4-ylmethyl)-3-(4- | 1-(2-methylpyrid-4-ylmethyl)-3-(4-trifluoromethoxy- |
| trifluoromethoxyphenyl)imidazolidine-2,4-dione | phenyl)imidazolidine-2,4-dione |
| 1-(2-ethylpyrid-4-ylmethyl)-3-(4-trifluoromethoxy- | 1-(2-isopropylpyrid-4-ylmethyl)-3-(4-trifluoromethoxy- |
| phenyl)imidazolidine-2,4-dione | phenyl)imidazolidine-2,4-dione |
| 1-(2-tert-butylpyrid-4-ylmethyl)-3-(4- | 1-(2-isobutylpyrid-4-ylmethyl)-3-(4-trifluoromethoxy- |
| trifluoromethoxyphenyl)imidazolidine-2,4-dione | phenyl)imidazolidine-2,4-dione |
| 1-(2-cyclohexylpyrid-4-ylmethyl)-3-(4-trifluoro- | 1-(2-phenylpyrid-4-ylmethyl)-3-(4-trifluoromethoxy- |
| methoxyphenyl)imidazolidine-2,4-dione | phenyl)imidazolidine-2,4-dione |
| 1-[2-(4-chlorophenyl)pyrid-4-ylmethyl]-3-(4- | 1-[2-(3-chlorophenyl)pyrid-4-ylmethyl]-3-(4-trifluoro- |
| trifluoromethoxyphenyl)imidazolidine-2,4-dione | methoxyphenyl)imidazolidine-2,4-dione |
| 1-[2-(2-chlorophenyl)pyrid-4-ylmethyl]-3-(4- | 1-[2-(4-fluorophenyl)pyrid-4-ylmethyl]-3-(4-trifluoro- |
| trifluoromethoxyphenyl)imidazolidine-2,4-dione | methoxyphenyl)imidazolidine-2,4-dione |
| 1-[2-(4-methoxyphenyl)pyrid-4-ylmethyl]-3-(4- | 1-(2-benzylpyrid-4-ylmethyl)-3-(4-trifluoromethoxy- |
| trifluoromethoxyphenyl)imidazolidine-2,4-dione | phenyl)imidazolidine-2,4-dione |
| 1-(2,2′-bipyrid-4-ylmethyl)-3-(4-trifluoromethoxy- | 1-(2-hydroxymethylpyrid-4-ylmethyl)-3-(4-trifluoro- |
| phenyl)imidazolidine-2,4-dione | methoxyphenyl)imidazolidine-2,4-dione |
| 1-(2-chloropyrid-4-ylmethyl)-3-(4-trifluoromethoxy- | 1-(2-fluoropyrid-4-ylmethyl)-3-(4-trifluoromethoxy- |
| phenyl)imidazolidine-2,4-dione | phenyl)imidazolidine-2,4-dione |
| 1-(2-bromopyrid-4-ylmethyl)-3-(4- | 1-(2-aminopyrid-4-ylmethyl)-3-(4-trifluoromethoxy- |
| trifluoromethoxyphenyl)imidazolidine-2,4-dione | phenyl)imidazolidine-2,4-dione |
| 1-(2-methylaminopyrid-4-ylmethyl)-3-(4-trifluoro- | 1-(2-ethylaminopyrid-4-ylmethyl)-3-(4- |
| methoxyphenyl)imidazolidine-2,4-dione | trifluoromethoxyphenyl)imidazolidine-2,4-dione |
| 1-(2-isopropylaminopyrid-4-ylmethyl)-3-(4- | 1-(2-phenylaminopyrid-4-ylmethyl)-3-(4-trifluoro- |
| trifluoromethoxyphenyl)imidazolidine-2,4-dione | methoxyphenyl)imidazolidine-2,4-dione |
| 1-(2-cyclohexylaminopyrid-4-ylmethyl)-3-(4- | 1-(2-diethylaminopyrid-4-ylmethyl)-3-(4-trifluoro- |
| trifluoromethoxyphenyl)imidazolidine-2,4-dione | methoxyphenyl)imidazolidine-2,4-dione |
| 1-(2-N-cyclohexyl-N-methylaminopyrid-4- | 1-(2-piperidinopyrid-4-ylmethyl)-3-(4- |
| ylmethyl)-3-(4- | trifluoromethoxyphenyl)imidazolidine-2,4-dione |
| trifluoromethoxyphenyl)imidazolidine-2,4-dione | |
| 1-(2-morpholinopyrid-4-ylmethyl)-3-(4- | 1-[2-(4-methylpiperazino)pyrid-4-ylmethyl]-3-(4- |
| trifluoromethoxyphenyl)imidazolidine-2,4-dione | trifluoromethoxyphenyl)imidazolidine-2,4-dione |
| 1-[2-(1H-pyrazol-1-yl)pyrid-4-ylmethyl]-3-(4- | 1-[2-(2-benzoylhydrazino)pyrid-4-ylmethyl]-3-(4- |
| trifluoromethoxyphenyl)imidazolidine-2,4-dione | trifluoromethoxyphenyl)imidazolidine-2,4-dione |
| 1-(2-benzoylaminopyrid-4-ylmethyl)-3-(4-trifluoro- | 1-[2-(4-chlorobenzoylamino)pyrid-4-ylmethyl]-3-(4- |
| methoxyphenyl)imidazolidine-2,4-dione | trifluoromethoxyphenyl)imidazolidine-2,4-dione |
| 1-[2-(3-chlorobenzoylamino)pyrid-4-ylmethyl]-3-(4- | 1-[2-(2-chlorobenzoylamino)pyrid-4-ylmethyl]-3-(4- |
| trifluoromethoxyphenyl)imidazolidine-2,4-dione | trifluoromethoxyphenyl)imidazolidine-2,4-dione |
| 1-[2-(4-fluorobenzoylamino)pyrid-4-ylmethyl]-3-(4- | 1-[2-(4-methoxybenzoylamino)pyrid-4-ylmethyl]-3-(4- |
| trifluoromethoxyphenyl)imidazolidine-2,4-dione | trifluoromethoxyphenyl)imidazolidine-2,4-dione |
| 1-(2-acetamidopyrid-4-ylmethyl)-3-(4- | 1-(2-phenylacetamidopyrid-4-ylmethyl)-3-(4-trifluoro- |
| trifluoromethoxyphenyl)imidazolidine-2,4-dione | methoxyphenyl)imidazolidine-2,4-dione |
| 1-[2-(4-methylpiperazino)acetamidopyrid-4- | 1-(2-cyclohexanylcarboxamidopyrid-4-ylmethyl)-3-(4- |
| ylmethyl]-3-(4- | trifluoromethoxyphenyl)imidazolidine-2,4-dione |
| trifluoromethoxyphenyl)imidazolidine-2,4-dione | |
| 1-[2-(1-methyl-4-piperidinyl)carbonylaminopyrid-4- | 1-[2-(2-furanylcarbonyl)aminopyrid-4-ylmethyl]-3-(4- |
| ylmethyl]-3-(4- | trifluoromethoxyphenyl)imidazolidine-2,4-dione |
| trifluoromethoxyphenyl)imidazolidine-2,4-dione | |
| 1-(2-ethoxycarbonylaminopyrid-4-ylmethyl)-3-(4- | 1-(2-isopropoxycarbonylaminopyrid-4-ylmethyl)-3-(4- |
| trifluoromethoxyphenyl)imidazolidine-2,4-dione | trifluoromethoxyphenyl)imidazolidine-2,4-dione |
| 1-(2-butoxycarbonylaminopyrid-4-ylmethyl)-3-(4- | 1-[2-(2-diethylaminoethoxycarbonylamino)pyrid-4-yl- |
| trifluoromethoxyphenyl)imidazolidine-2,4-dione | methyl]-3-(4-trifluoromethoxyphenyl)imidazolidine- |
| | 2,4-dione |
| 1-(2-phenoxycarbonylaminopyrid-4-ylmethyl)-3-(4- | 1-[2-(4-chloroanilino)carbonylaminopyrid-4-ylmethyl]- |
| trifluoromethoxyphenyl)imidazolidine-2,4-dione | 3-(4-trifluoromethoxyphenyl)imidazolidine-2,4-dione |
| 1-(2-anilinocarbonylaminopyrid-4-ylmethyl)-3-(4- | 1-(2-benzylaminocarbonylaminopyrid-4-ylmethyl)-3- |
| trifluoromethoxyphenyl)imidazolidine-2,4-dione | (4-trifluoromethoxyphenyl)imidazolidine-2,4-dione |
| 1-[2-(2-pyridylamino)carbonylaminopyrid-4- | 1-(2-ureidopyrid-4-ylmethyl)-3-(4-trifluoromethoxy- |
| ylmethyl]-3-(4- | phenyl)imidazolidine-2,4-dione |
| trifluoromethoxyphenyl)imidazolidine-2,4-dione | |
| 1-(2-benzylaminosulfonylaminopyrid-4-ylmethyl)- | 1-(2-methylaminosulfonylaminopyrid-4-ylmethyl)-3- |
| 3-(4-trifluoromethoxyphenyl)imidazolidine-2,4- | (4-trifluoromethoxyphenyl)imidazolidine-2,4-dione |
| dione | |
| 1-(2-aminosulfonylaminopyrid-4-ylmethyl)-3-(4- | 1-(2-morpholinosulfonylaminopyrid-4-ylmethyl)-3-(4- |
| trifluoromethoxyphenyl)imidazolidine-2,4-dione | trifluoromethoxyphenyl)imidazolidine-2,4-dione |
| 1-(2-dimethylaminosulfonylaminopyrid-4- | 1-(2-anilinosulfonylaminopyrid-4-ylmethyl)-3-(4- |
| ylmethyl)-3-(4- | trifluoromethoxyphenyl)imidazolidine-2,4-dione |
| trifluoromethoxyphenyl)imidazolidine-2,4-dione | |
| 1-(2-phenylsulfonylaminopyrid-4-ylmethyl)-3-(4- | 1-[2-(2-thienyl)sulfonylaminopyrid-4-ylmethyl]-3-(4- |
| trifluoromethoxyphenyl)imidazolidine-2,4-dione | trifluoromethoxyphenyl)imidazolidine-2,4-dione |
| 1-(2-methylsulfonylaminopyrid-4-ylmethyl)-3-(4- | 1-[2-(4-chlorophenyl)sulfonylaminopyrid-4-ylmethyl]- |
| trifluoromethoxyphenyl)imidazolidine-2,4-dione | 3-(4-trifluoromethoxyphenyl)imidazolidine-2,4-dione |
| 1-(2-benzylsulfonylaminopyrid-4-ylmethyl)-3-(4- | 1-(2-hydroxycarbonylpyrid-4-ylmethyl)-3-(4-trifluoro- |
| trifluoromethoxyphenyl)imidazolidine-2,4-dione | methoxyphenyl)imidazolidine-2,4-dione |
| 1-(2-ethoxycarbonylpyrid-4-ylmethyl)-3-(4- | 1-(2-tert-butoxycarbonylpyrid-4-ylmethyl)-3-(4- |
| trifluoromethoxyphenyl)imidazolidine-2,4-dione | trifluoromethoxyphenyl)imidazolidine-2,4-dione |
| 1-(2-phenoxycarbonylpyrid-4-ylmethyl)-3-(4- | 1-[2-(4-methylpiperazinocarbonyl)pyrid-4-ylmethyl]-3- |
| trifluoromethoxyphenyl)imidazolidine-2,4-dione | (4-trifluoromethoxyphenyl)imidazolidine-2,4-dione |
| 1-[2-(methylamino)carbonylpyrid-4-ylmethyl]-3-(4- | 1-[2-(dimethylamino)carbonylpyrid-4-ylmethyl]-3-(4- |
| trifluoromethoxyphenyl)imidazolidine-2,4-dione | trifluoromethoxyphenyl)imidazolidine-2,4-dione |
| 1-(2-piperidinocarbonylpyrid-4-ylmethyl)-3-(4- | 1-(2-morpholinocarbonylpyrid-4-ylmethyl)-3-(4- |
| trifluoromethoxyphenyl)imidazolidine-2,4-dione | trifluoromethoxyphenyl)imidazolidine-2,4-dione |
| 1-[2-(4-phenylpiperazinocarbonyl)pyrid-4- | 1-(2-phenylaminocarbonylpyrid-4-ylmethyl)-3-(4- |
| ylmethyl]-3-(4- | trifluoromethoxyphenyl)imidazolidine-2,4-dione |
| trifluoromethoxyphenyl)imidazolidine-2,4-dione | |
| 1-[2-(methylphenylamino)carbonylpyrid-4- | 1-[2-(4-methoxyphenyl)methylaminocarbonylpyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 1-[2-(4-chlorophenyl)methylaminocarbonylpyrid-4- | 1-[2-(4-morpholinylamino)carbonylpyrid-4-ylmethyl]- |
| ylmethyl]-3-(4- | 3-(4-trifluoromethoxyphenyl)imidazolidine-2,4-dione |
| trifluoromethoxyphenyl)imidazolidine-2,4-dione | |
| 1-(2-aminocarbonylpyrid-4-ylmethyl)-3-(4-trifluoro- | 1-(2-phenoxypyrid-4-ylmethyl)-3-(4-trifluoromethoxy- |
| methoxyphenyl)imidazolidine-2,4-dione | phenyl)imidazolidine-2,4-dione |
| 1-(2-methoxypyrid-4-ylmethyl)-3-(4- | 1-(2-ethoxypyrid-4-ylmethyl)-3-(4-trifluoromethoxy- |
| trifluoromethoxyphenyl)imidazolidine-2,4-dione | phenyl)imidazolidine-2,4-dione |
| 1-[2-(1-pyrrolidinyl)ethoxypyrid-4-ylmethyl]-3-(4- | 1-(2-benzyloxypyrid-4-ylmethyl)-3-(4- |
| trifluoromethoxyphenyl)imidazolidine-2,4-dione | trifluoromethoxyphenyl)imidazolidine-2,4-dione |
| 1-(2-benzoyloxypyrid-4-ylmethyl)-3-(4- | 1-[2-(4,5-dihydro-1H-imidazol-2-yl)pyrid-4-ylmethyl]- |
| trifluoromethoxyphenyl)imidazolidine-2,4-dione | 3-(4-trifluoromethoxyphenyl)imidazolidine-2,4-dione |
| 1-[2-(1H-imidazol-2-yl)pyrid-4-ylmethyl]-3-(4- | 1-[2-(1H-imidazol-2-yl)pyrid-4-ylmethyl]-3-(4- |
| trifluoromethoxyphenyl)imidazolidine-2,4-dione | trifluoromethoxyphenyl)imidazolidine-2,4-dione |
| 1-[2-(1H-tetrazol-5-yl)pyrid-4-ylmethyl]-3-(4- | 1-(2-amidinopyrid-4-ylmethyl)-3-(4- |
| trifluoromethoxyphenyl)imidazolidine-2,4-dione | trifluoromethoxyphenyl)imidazolidine-2,4-dione |
| 1-(2-dimethylaminomethylpyrid-4-ylmethyl)-3-(4- | 1-(2-aminomethylpyrid-4-ylmethyl)-3-(4-trifluoro- |
| trifluoromethoxyphenyl)imidazolidine-2,4-dione | methoxyphenyl)imidazolidine-2,4-dione |
| 1-(3-methylpyrid-4-ylmethyl)-3-(4- | 1-(3-ethylpyrid-4-ylmethyl)-3-(4-trifluoromethoxy- |
| trifluoromethoxyphenyl)imidazolidine-2,4-dione | phenyl)imidazolidine-2,4-dione |
| 1-(3-isopropylpyrid-4-ylmethyl)-3-(4- | 1-(3-tert-butylpyrid-4-ylmethyl)-3-(4- |
| trifluoromethoxyphenyl)imidazolidine-2,4-dione | trifluoromethoxyphenyl)imidazolidine-2,4-dione |
| 1-(3-isobutylpyrid-4-ylmethyl)-3-(4- | 1-(3-cyclohexylpyrid-4-ylmethyl)-3-(4- |
| trifluoromethoxyphenyl)imidazolidine-2,4-dione | trifluoromethoxyphenyl)imidazolidine-2,4-dione |
| 1-(3-phenylpyrid-4-ylmethyl)-3-(4- | 1-[3-(4-chlorophenyl)pyrid-4-ylmethyl]-3-(4-trifluoro- |
| trifluoromethoxyphenyl)imidazolidine-2,4-dione | methoxyphenyl)imidazolidine-2,4-dione |
| 1-[3-(3-chlorophenyl)pyrid-4-ylmethyl]-3-(4- | 1-[3-(2-chlorophenyl)pyrid-4-ylmethyl]-3-(4- |
| trifluoromethoxyphenyl)imidazolidine-2,4-dione | trifluoromethoxyphenyl)imidazolidine-2,4-dione |
| 1-[3-(4-fluorophenyl)pyrid-4-ylmethyl]-3-(4- | 1-(3-benzylpyrid-4-ylmethyl)-3-(4-trifluoromethoxy- |
| trifluoromethoxyphenyl)imidazolidine-2,4-dione | phenyl)imidazolidine-2,4-dione |
| 1-(3,2′-bipyrid-4-ylmethyl)-3-(4-trifluoromethoxy- | 1-(3-hydroxymethylpyrid-4-ylmethyl)-3-(4-trifluoro- |
| phenyl)imidazolidine-2,4-dione | methoxyphenyl)imidazolidine-2,4-dione |
| 1-(3-chloropyrid-4-ylmethyl)-3-(4-trifluoromethoxy- | 1-(3-fluoropyrid-4-ylmethyl)-3-(4-trifluoromethoxy- |
| phenyl)imidazolidine-2,4-dione | phenyl)imidazolidine-2,4-dione |
| 1-(3-bromopyrid-4-ylmethyl)-3-(4- | 1-(3-aminopyrid-4-ylmethyl)-3-(4-trifluoromethoxy- |
| trifluoromethoxyphenyl)imidazolidine-2,4-dione | phenyl)imidazolidine-2,4-dione |
| 1-(3-methylaminopyrid-4-ylmethyl)-3-(4-trifluoro- | 1-(3-ethylaminopyrid-4-ylmethyl)-3-(4-trifluoro- |
| methoxyphenyl)imidazolidine-2,4-dione | methoxyphenyl)imidazolidine-2,4-dione |
| 1-(3-isopropylaminopyrid-4-ylmethyl)-3-(4- | 1-(3-phenylaminopyrid-4-ylmethyl)-3-(4-trifluoro- |
| trifluoromethoxyphenyl)imidazolidine-2,4-dione | methoxyphenyl)imidazolidine-2,4-dione |
| 1-(3-cyclohexylaminopyrid-4-ylmethyl)-3-(4- | 1-(3-diethylaminopyrid-4-ylmethyl)-3-(4-trifluoro- |
| trifluoromethoxyphenyl)imidazolidine-2,4-dione | methoxyphenyl)imidazolidine-2,4-dione |
| 1-(3-N-cyclohexyl-N-methylaminopyrid-4- | 1-(3-piperidinopyrid-4-ylmethyl)-3-(4-trifluoro- |
| ylmethyl)-3-(4- | methoxyphenyl)imidazolidine-2,4-dione |
| trifluoromethoxyphenyl)imidazolidine-2,4-dione | |
| 1-(3-morpholinopyrid-4-ylmethyl)-3-(4-trifluoro- | 1-[3-(4-methylpiperazino)pyrid-4-ylmethyl]-3-(4- |
| methoxyphenyl)imidazolidine-2,4-dione | trifluoromethoxyphenyl)imidazolidine-2,4-dione |
| 1-[3-(1H-pyrazol-1-yl)pyrid-4-ylmethyl]-3-(4- | 1-[3-(2-benzoylhydrazino)pyrid-4-ylmethyl]-3-(4- |
| trifluoromethoxyphenyl)imidazolidine-2,4-dione | trifluoromethoxyphenyl)imidazolidine-2,4-dione |
| 1-(3-benzoylaminopyrid-4-ylmethyl)-3-(4-trifluoro- | 1-[3-(4-chlorobenzoylamino)pyrid-4-ylmethyl]-3-(4- |
| methoxyphenyl)imidazolidine-2,4-dione | trifluoromethoxyphenyl)imidazolidine-2,4-dione |
| 1-[3-(3-chlorobenzoylamino)pyrid-4-ylmethyl]-3-(4- | 1-[3-(2-chlorobenzoylamino)pyrid-4-ylmethyl]-3-(4- |
| trifluoromethoxyphenyl)imidazolidine-2,4-dione | trifluoromethoxyphenyl)imidazolidine-2,4-dione |
| 1-[3-(4-fluoroberizoylamino)pyrid-4-ylmethyl]-3-(4- | 1-[3-(4-methoxybenzoylamino)pyrid-4-ylmethyl]-3-(4- |
| trifluoromethoxyphenyl)imidazolidine-2,4-dione | trifluoromethoxyphenyl)imidazolidine-2,4-dione |
| 1-(3-acetamidopyrid-4-ylmethyl)-3-(4-trifluoro- | 1-(3-phenylacetamidopyrid-4-ylmethyl)-3-(4-trifluoro- |
| methoxyphenyl)imidazolidine-2,4-dione | methoxyphenyl)imidazolidine-2,4-dione |
| 1-[3-(4-methylpiperazino)acetamidopyrid-4- | 1-(3-cyclohexanylcarboxamidopyrid-4-ylmethyl)-3-(4- |
| ylmethyl]-3-(4- | trifluoromethoxyphenyl)imidazolidine-2,4-dione |
| trifluoromethoxyphenyl)imidazolidine-2,4-dione | |
| 1-[3-(1-methyl-4-piperidiriyl)carbonylaminopyrid-4- | 1-[3-(2-furanylcarbonyl)aminopyrid-4-ylmethyl]-3-(4- |
| ylmethyl]-3-(4- | trifluoromethoxyphenyl)imidazolidine-2,4-dione |
| trifluoromethoxyphenyl)imidazolidine-2,4-dione | |
| 1-(3-ethoxycarbonylaminopyrid-4-ylmethyl)-3-(4- | 1-(3-isopropoxycarbonylaminopyrid-4-ylmethyl)-3-(4- |
| trifluoromethoxyphenyl)imidazolidine-2,4-dione | trifluoromethoxyphenyl)imidazolidine-2,4-dione |
| 1-(3-butoxycarbonylaminopyrid-4-ylmethyl)-3-(4- | 1-[3-(2-diethylaminoethoxycarbonylamino)pyrid-4- |
| trifluoromethoxyphenyl)imidazolidine-2,4-dione | ylmethyl]-3-(4-trifluoromethoxyphenyl)imidazolidine- |
| | 2,4-dione |
| 1-(3-phenoxycarbonylaminopyrid-4-ylmethyl)-3-(4- | 1-[3-(4-chloroanilino)carbonylaminopyrid-4-ylmethyl]- |
| trifluoromethoxyphenyl)imidazolidine-2,4-dione | 3-(4-trifluoromethoxyphenyl)imidazolidine-2,4-dione |
| 1-(3-anilinocarbonylaminopyrid-4-ylmethyl)-3-(4- | 1-(3-benzylaminocarbonylaminopyrid-4-ylmethyl)-3- |
| trifluoromethoxyphenyl)imidazolidine-2,4-dione | (4-trifluoromethoxyphenyl)imidazolidine-2,4-dione |
| 1-[3-(2-pyridylamino)carbonylaminopyrid-4- | 1-(3-ureidopyrid-4-ylmethyl)-3-(4-trifluoromethoxy- |
| ylmethyl]-3-(4- | phenyl)imidazolidine-2,4-dione |
| trifluoromethoxyphenyl)imidazolidine-2,4-dione | |
| 1-(3-benzylaminosulfonylaminopyrid-4-ylmethyl)- | 1-(3-methylaminosulfonylaminopyrid-4-ylmethyl)-3- |
| 3-(4-trifluoromethoxyphenyl)imidazolidine-2,4- | (4-trifluoromethoxyphenyl)imidazolidine-2,4-dione |
| dione | |
| 1-(3-aminosulfonylaminopyrid-4-ylmethyl)-3-(4- | 1-(3-morpholinosulfonylaminopyrid-4-ylmethyl)-3-(4- |
| trifluoromethoxyphenyl)imidazolidine-2,4-dione | trifluoromethoxyphenyl)imidazolidine-2,4-dione |
| 1-(3-dimethylaminosulfonylaminopyrid-4- | 1-(3-anilinosulfonylaminopyrid-4-ylmethyl)-3-(4- |
| ylmethyl)-3-(4- | trifluoromethoxyphenyl)imidazolidine-2,4-dione |
| trifluoromethoxyphenyl)imidazolidine-2,4-dione | |
| 1-(3-phenylsulfonylaminopyrid-4-ylmethyl)-3-(4- | 1-[3-(2-thienyl)sulfonylaminopyrid-4-ylmethyl]-3-(4- |
| trifluoromethoxyphenyl)imidazolidine-2,4-dione | trifluoromethoxyphenyl)imidazolidine-2,4-dione |
| 1-(3-methylsulfonylaminopyrid-4-ylmethyl)-3-(4- | 1-[3-(4-chlorophenyl)sulfonylaminopyrid-4-ylmethyl]- |
| trifluoromethoxyphenyl)imidazolidine-2,4-dione | 3-(4-trifluoromethoxyphenyl)imidazolidine-2,4-dione |
| 1-(3-benzylsulfonylaminopyrid-4-ylmethyl)-3-(4- | 1-(3-hydroxycarbonylpyrid-4-ylmethyl)-3-(4-trifluoro- |
| trifluoromethoxyphenyl)imidazolidine-2,4-dione | methoxyphenyl)imidazolidine-2,4-dione |
| 1-(3-ethoxycarbonylpyrid-4-ylmethyl)-3-(4- | 1-(3-tert-butoxycarbonylpyrid-4-ylmethyl)-3-(4- |
| trifluoromethoxyphenyl)imidazolidine-2,4-dione | trifluoromethoxyphenyl)imidazolidine-2,4-dione |
| 1-(3-phenoxycarbonylpyrid-4-ylmethyl)-3-(4- | 1-[3-(4-methylpiperazinocarbonyl)pyrid-4-ylmethyl]-3- |
| trifluoromethoxyphenyl)imidazolidine-2,4-dione | (4-trifluoromethoxyphenyl)imidazolidine-2,4-dione |
| 1-[3-(methylamino)carbonylpyrid-4-ylmethyl]-3-(4- | 1-[3-(dimethylamino)carbonylpyrid-4-ylmethyl]-3-(4- |
| trifluoromethoxyphenyl)imidazolidine-2,4-dione | trifluoromethoxyphenyl)imidazolidine-2,4-dione |
| 1-(3-piperidinocarbonylpyrid-4-ylmethyl)-3-(4- | 1-(3-morpholinocarbonylpyrid-4-ylmethyl)-3-(4- |
| trifluoromethoxyphenyl)imidazolidine-2,4-dione | trifluoromethoxyphenyl)imidazolidine-2,4-dione |
| 1-[3-(4-phenylpiperazinocarbonyl)pyrid-4- | 1-(3-phenylaminocarbonylpyrid-4-ylmethyl)-3-(4- |
| ylmethyl]-3-(4- | trifluoromethoxyphenyl)imidazolidine-2,4-dione |
| trifluoromethoxyphenyl)imidazolidine-2,4-dione | |
| 1-[3-(methylphenylamino)carbonylpyrid-4- | 1-[3-(4-methoxyphenyl)methylaminocarbonylpyrid-4- |
| ylmethyl]-3-(4- | ylmethyl]-3-(4-trifluoromethoxyphenyl)imidazolidine- |
| trifluoromethoxyphenyl)imidazolidine-2,4-dione | 2,4-dione |
| 1-[3-(4-chlorophenyl)methylaminocarbonylpyrid-4- | 1-[3-(4-morpholinylamino)carbonylpyrid-4-ylmethyl]- |
| ylmethyl]-3-(4- | 3-(4-trifluoromethoxyphenyl)imidazolidine-2,4-dione |
| trifluoromethoxyphenyl)imidazolidine-2,4-dione | |
| 1-(3-aminocarbonylpyrid-4-ylmethyl)-3-(4-trifluoro- | 1-(3-phenoxypyrid-4-ylmethyl)-3-(4-trifluoromethoxy- |
| methoxyphenyl)imidazolidine-2,4-dione | phenyl)imidazolidine-2,4-dione |
| 1-(3-methoxypyrid-4-ylmethyl)-3-(4-trifluoro- | 1-(3-ethoxypyrid-4-ylmethyl)-3-(4-trifluoromethoxy- |
| methoxyphenyl)imidazolidine-2,4-dione | phenyl)imidazolidine-2,4-dione |
| 1-[3-(1-pyrrolidinyl)ethoxypyrid-4-ylmethyl]-3-(4- | 1-(3-benzyloxypyrid-4-ylmethyl)-3-(4- |
| trifluoromethoxyphenyl)imidazolidine-2,4-dione | trifluoromethoxyphenyl)imidazolidine-2,4-dione |
| 1-(3-benzoyloxypyrid-4-ylmethyl)-3-(4- | 1-[3-(4,5-dihydro-1H-imidazol-2-yl)pyrid-4-ylmethyl]- |
| trifluoromethoxyphenyl)imidazolidine-2,4-dione | 3-(4-trifluoromethoxyphenyl)imidazolidine-2,4-dione |
| 1-[3-(1H-imidazol-2-yl)pyrid-4-ylmethyl]-3-(4- | 1-[3-(1H-tetrazol-5-yl)pyrid-4-ylmethyl]-3-(4-trifluoro- |
| trifluoromethoxyphenyl)imidazolidine-2,4-dione | methoxyphenyl)imidazolidine-2,4-dione |
| 1-(3-amidinopyrid-4-ylmethyl)-3-(4- | 1-(3-dimethylaminomethylpyridin)-4-ylmethyl]-3-(4- |
| trifluoromethoxyphenyl)imidazolidine-2,4-dione | trifluoromethoxyphenyl)imidazolidine-2,4-dione |
| 1-(3-aminomethylpyrid-4-ylmethyl)-3-(4- | 5,5-dimethyl-1-(2-methylpyrid-4-ylmethyl)-3-(4- |
| trifluoromethoxyphenyl)imidazolidine-2,4-dione | trifluoromethanesulfanylphenyl)imidazolidine-2,4- |
| | dione |
| 5,5-dimethyl-1-(2-ethylpyrid-4-ylmethyl)-3-(4- | 5,5-dimethyl-1-(2-isopropylpyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-tert-butylpyrid-4-ylmethyl)-3-(4- | 5,5-dimethyl-1-(2-isobutylpyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-cyclohexylpyrid-4-ylmethyl)-3- | 5,5-dimethyl-1-(2-phenylpyrid-4-ylmethyl)-3-(4- |
| (4-trifluoromethanesulfanylphenyl)imidazolidine- | trifluoromethanesulfanylphenyl)imidazolidine-2,4- |
| 2,4-dione | dione |
| 5,5-dimethyl-1-[2-(4-chlorophenyl)pyrid-4- | 5,5-dimethyl-1-[2-(3-chlorophenyl)pyrid-4-ylmethyl]- |
| ylmethyl]-3-(4- | 3-(4-trifluoromethanesulfanylphenyl)imidazolidine-2,4- |
| trifluoromethanesulfanylphenyl)imidazolidine-2,4- | dione |
| dione | |
| 5,5-dimethyl-1-[2-(4-fluorophenyl)pyrid-4- | 5,5-dimethyl-1-[2-(4-methoxyphenyl)pyrid-4- |
| ylmethyl]-3-(4- | ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-benzylpyrid-4-ylmethyl)-3-(4- | 5,5-dimethyl-1-(2,2′-bipyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-hydroxymethylpyrid-4-ylmethyl)- | 5,5-dimethyl-1-(2-chloropyrid-4-ylmethyl)-3-(4- |
| 3-(4-trifluoromethanesulfanylphenyl)imidazolidine- | trifluoromethanesulfanylphenyl)imidazolidine-2,4- |
| 2,4-dione | dione |
| 5,5-dimethyl-1-(2-fluoropyrid-4-ylmethyl)-3-(4- | 5,5-dimethyl-1-(2-bromopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-aminopyrid-4-ylmethyl)-3-(4- | 5,5-dimethyl-1-(2-methylaminopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-ethylaminopyrid-4-ylmethyl)-3- | 5,5-dimethyl-1-(2-isopropylaminopyrid-4-ylmethyl)-3- |
| (4-trifluoromethanesulfanylphenyl)imidazolidine- | (4-trifluoromethanesulfanylphenyl)imidazolidine-2,4- |
| 2,4-dione | dione |
| 5,5-dimethyl-1-(2-phenylaminopyrid-4-ylmethyl)-3- | 5,5-dimethyl-1-(2-cyclohexylaminopyrid-4-ylmethyl)- |
| (4-trifluoromethanesulfanylphenyl)imidazolidine- | 3-(4-trifluoromethanesulfanylphenyl)imidazolidine-2,4- |
| 2,4-dione | dione |
| 5,5-dimethyl-1-(2-diethylaminopyrid-4-ylmethyl)-3- | 5,5-dimethyl-1-(2-N-cyclohexyl-N-methylaminopyrid- |
| (4-trifluoromethanesulfanylphenyl)imidazolidine- | 4-ylmethyl)-3-(4-trifluoromethanesulfanylphenyl)imid- |
| 2,4-dione | azolidine-2,4-dione |
| 5,5-dimethyl-1-(2-piperidinopyrid-4-ylmethyl)-3-(4- | 5,5-dimethyl-1-(2-morpholinopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(4-methylpiperazino)pyrid-4-yl- | 5,5-dimethyl-1-[2-(1H-pyrazol-1-yl)pyrid-4-ylmethyl]- |
| methyl]-3-(4- | 3-(4-trifluoromethanesulfanylphenyl)imidazolidine-2,4- |
| trifluoromethanesulfanylphenyl)imidazolidine-2,4- | dione |
| dione | |
| 5,5-dimethyl-1-[2-(2-benzoylhydrazino)pyrid-4-yl- | 5,5-dimethyl-1-(2-benzoylaminopyrid-4-ylmethyl)-3- |
| methyl]-3-(4- | (4-trifluoromethanesulfanylphenyl)imidazolidine-2,4- |
| trifluoromethanesulfanylphenyl)imidazolidine-2,4- | dione |
| dione | |
| 5,5-dimethyl-1-[2-(4-chlorobenzoylamino)pyrid-4- | 5,5-dimethyl-1-[2-(3-chlorobenzoylamino)pyrid-4-yl- |
| ylmethyl]-3-(4- | methyl]-3-(4-trifluoromethanesulfanylphenyl)imidazol- |
| trifluoromethanesulfanylphenyl)imidazolidine-2,4- | idine-2,4-dione |
| dione | |
| 5,5-dimethyl-1-[2-(2-chlorobenzoylamino)pyrid-4- | 5,5-dimethyl-1-[2-(4-fluorobenzoylamino)pyrid-4- |
| ylmethyl]-3-(4- | ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(4-methoxybenzoylamino)pyrid- | 5,5-dimethyl-1-(2-acetamidopyrid-4-ylmethyl)-3-(4- |
| 4-ylmethyl]-3-(4- | trifluoromethanesulfanylphenyl)imidazolidine-2,4- |
| trifluoromethanesulfanylphenyl)imidazolidine-2,4- | dione |
| dione | |
| 5,5-dimethyl-1-(2-phenylacetamidopyrid-4- | 5,5-dimethyl-1-[2-(4- |
| ylmethyl)-3-(4- | methylpiperazino)acetamidopyrid-4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-cyclohexanylcarboxamidopyrid- | 5,5-dimethyl-1-[2-(1-methyl-4-piperidinyl)carbonyl- |
| 4-ylmethyl)-3-(4- | aminopyrid-4-ylmethyl]-3-(4-trifluoromethanesulfanyl- |
| trifluoromethanesulfanylphenyl)imidazolidine-2,4- | phenyl)imidazolidine-2,4-dione |
| dione | |
| 5,5-dimethyl-1-[2-(2-furanylcarbonyl)aminopyrid-4- | 5,5-dimethyl-1-(2-ethoxycarbonylaminopyrid-4- |
| ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-isopropoxycarbonylaminopyrid- | 5,5-dimethyl-1-(2-butoxycarbonylaminopyrid-4- |
| 4-ylmethyl)-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(2- | 5,5-dimethyl-1-(2-phenoxycarbonylaminopyrid-4-yl- |
| diethylaminoethoxycarbonylamino)pyrid-4- | methyl)-3-(4-trifluoromethanesulfanylphenyl)imidazol- |
| ylmethyl]-3-(4-trifluoromethanesulfanylphenyl)- | idine-2,4-dione |
| imidazolidine-2,4-dione | |
| 5,5-dimethyl-1-[2-(4- | 5,5-dimethyl-1-(2-anilinocarbonylaminopyrid-4-yl- |
| chloroanilino)carbonylaminopyrid-4-ylmethyl]-3-(4- | methyl)-3-(4-trifluoromethanesulfanylphenyl)imidazol- |
| trifluoromethanesulfanylphenyl)imidazolidine-2,4- | idine-2,4-dione |
| dione | |
| 5,5-dimethyl-1-(2-benzylaminocarbonylaminopyrid- | 5,5-dimethyl-1-[2-(2- |
| 4-ylmethyl)-3-(4- | pyridylamino)carbonylaminopyrid-4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-ureidopyrid-4-ylmethyl)-3-(4- | 5,5-dimethyl-1-(2-benzylaminosulfonylaminopyrid-4- |
| trifluoromethanesulfanylphenyl)imidazolidine-2,4- | ylmethyl)-3-(4- |
| dione | trifluoromethanesulfanylphenyl)imidazolidine-2,4- |
| | dione |
| 5,5-dimethyl-1-(2-methylaminosulfonylaminopyrid- | 5,5-dimethyl-1-(2-aminosulfonylaminopyrid-4- |
| 4-ylmethyl)-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-morpholinosulfonylaminopyrid-4- | 5,5-dimethyl-1-(2-dimethylaminosulfonylaminopyrid- |
| ylmethyl)-3-(4- | 4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-anilinosulfonylaminopyrid-4-yl- | 5,5-dimethyl-1-(2-phenylsulfonylaminopyrid-4- |
| methyl)-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(2-thienyl)sulfonylaminopyrid-4- | 5,5-dimethyl-1-(2-methylsulfonylaminopyrid-4- |
| ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(4- | 5,5-dimethyl-1-(2-benzylsulfonylaminopyrid-4- |
| chlorophenyl)sulfonylaminopyrid-4-ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-hydroxycarbonylpyrid-4- | 5,5-dimethyl-1-(2-ethoxycarbonylpyrid-4-ylmethyl)-3- |
| ylmethyl)-3-(4- | (4-trifluoromethanesulfanylphenyl)imidazolidine-2,4- |
| trifluoromethanesulfanylphenyl)imidazolidine-2,4- | dione |
| dione | |
| 5,5-dimethyl-1-(2-tert-butoxycarbonylpyrid-4- | 5,5-dimethyl-1-(2-phenoxycarbonylpyrid-4-ylmethyl)- |
| ylmethyl)-3-(4- | 3-(4-trifluoromethanesulfanylphenyl)imidazolidine-2,4- |
| trifluoromethanesulfanylphenyl)imidazolidine-2,4- | dione |
| dione | |
| 5,5-dimethyl-1-[2-(4- | 5,5-dimethyl-1-[2-(methylamino)carbonylpyrid-4-yl- |
| methylpiperazinocarbonyl)pyrid-4-ylmethyl]-3-(4- | methyl]-3-(4-trifluoromethanesulfanylphenyl)imidazol- |
| trifluoromethanesulfanylphenyl)imidazolidine-2,4- | idine-2,4-dione |
| dione | |
| 5,5-dimethyl-1-[2-(dimethylamino)carbonylpyrid-4- | 5,5-dimethyl-1-(2-piperidinocarbonylpyrid-4- |
| ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-morpholinocarbonylpyrid-4- | 5,5-dimethyl-1-[2-(4-phenylpiperazinocarbonyl)pyrid- |
| ylmethyl)-3-(4- | 4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-phenylaminocarbonylpyrid-4- | 5,5-dimethyl-1-[2-(methylphenylamino)carbonylpyrid- |
| ylmethyl)-3-(4- | 4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(4- | 5,5-dimethyl-1-[2-(4- |
| methoxyphenyl)methylaminocarbonylpyrid-4- | chlorophenyl)methylaminocarbonylpyrid-4-ylmethyl]- |
| ylmethyl]-3-(4-trifluoromethanesulfanylphenyl)- | 3-(4-trifluoromethanesulfanylphenyl)imidazolidine-2,4- |
| imidazolidine-2,4-dione | dione |
| 5,5-dimethyl-1-[2-(4- | 5,5-dimethyl-1-(2-aminocarbonylpyrid-4-ylmethyl)-3- |
| morpholinylamino)carbonylpyrid-4-ylmethyl]-3-(4- | (4-trifluoromethanesulfanylphenyl)imidazolidine-2,4- |
| trifluoromethanesulfanylphenyl)imidazolidine-2,4- | dione |
| dione | |
| 5,5-dimethyl-1-(2-phenoxypyrid-4-ylmethyl)-3-(4- | 5,5-dimethyl-1-(2-methoxypyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(2-ethoxypyrid-4-ylmethyl)-3-(4- | 5,5-dimethyl-1-[2-(1-pyrrolidinyl)ethoxypyrid-4- |
| trifluoromethanesulfanylphenyl)imidazolidine-2,4- | ylmethyl]-3-(4-trifluoromethanesulfanylphenyl)- |
| dione | imidazolidine-2,4-dione |
| 5,5-dimethyl-1-(2-benzyloxypyrid-4-ylmethyl)-3-(4- | 5,5-dimethyl-1-(2-benzoyloxypyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(4,5-dihydro-1H-imidazol-2- | 5,5-dimethyl-1-[2-(1H-imidazol-2-yl)pyrid-4- |
| yl)pyrid-4-ylmethyl]-3-(4- | ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[2-(1H-tetrazol-5-yl)pyrid-4- | 5,5-dimethyl-1-(2-amidinopyrid-4-ylmethyl)-3-(4- |
| ylmethyl]-3-(4- | trifluoromethanesulfanylphenyl)imidazolidine-2,4- |
| trifluoromethanesulfanylphenyl)imidazolidine-2,4- | dione |
| dione | |
| 5,5-dimethyl-1-(2-dimethylaminomethylpyrid-4- | 5,5-dimethyl-1-(2-aminomethylpyrid-4-ylmethyl)-3-(4- |
| ylmethyl)-3-(4- | trifluoromethanesulfanylphenyl)imidazolidine-2,4- |
| trifluoromethanesulfanylphenyl)imidazolidine-2,4- | dione |
| dione | |
| 5,5-dimethyl-1-(3-methylpyrid-4-ylmethyl)-3-(4- | 5,5-dimethyl-1-(3-ethylpyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-isopropylpyrid-4-ylmethyl)-3-(4- | 5,5-dimethyl-1-(3-tert-butylpyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-isobutylpyrid-4-ylmethyl)-3-(4- | 5,5-dimethyl-1-(3-cyclohexylpyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-phenylpyrid-4-ylmethyl)-3-(4- | 5,5-dimethyl-1-[3-(4-chlorophenyl)pyrid-4-ylmethyl]- |
| trifluoromethanesulfanylphenyl)imidazolidine-2,4- | 3-(4-trifluoromethanesulfanylphenyl)imidazolidine-2,4- |
| dione | dione |
| 5,5-dimethyl-1-[3-(3-chlorophenyl)pyrid-4- | 5,5-dimethyl-1-[3-(2-chlorophenyl)pyrid-4-ylmethyl]- |
| ylmethyl]-3-(4- | 3-(4-trifluoromethanesulfanylphenyl)imidazolidine-2,4- |
| trifluoromethanesulfanylphenyl)imidazolidine-2,4- | dione |
| dione | |
| 5,5-dimethyl-1-[3-(4-fluorophenyl)pyrid-4- | 5,5-dimethyl-1-[3-(4-methoxyphenyl)pyrid-4- |
| ylmethyl]-3-(4- | ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-benzylpyrid-4-ylmethyl)-3-(4- | 5,5-dimethyl-1-(3,2′-bipyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-hydroxymethylpyrid-4-ylmethyl)- | 5,5-dimethyl-1-(3-chloropyrid-4-ylmethyl)-3-(4- |
| 3-(4-trifluoromethanesulfanylphenyl)imidazolidine- | trifluoromethanesulfanylphenyl)imidazolidine-2,4- |
| 2,4-dione | dione |
| 5,5-dimethyl-1-(3-fluoropyrid-4-ylmethyl)-3-(4- | 5,5-dimethyl-1-(3-bromopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-aminopyrid-4-ylmethyl)-3-(4- | 5,5-dimethyl-1-(3-methylaminopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-ethylaminopyrid-4-ylmethyl)-3- | 5,5-dimethyl-1-(3-isopropylaminopyrid-4-ylmethyl)-3- |
| (4-trifluoromethanesulfanylphenyl)imidazolidine- | (4-trifluoromethanesulfanylphenyl)imidazolidine-2,4- |
| 2,4-dione | dione |
| 5,5-dimethyl-1-(3-phenylaminopyrid-4-ylmethyl)-3- | 5,5-dimethyl-1-(3-cyclohexylaminopyrid-4-ylmethyl)- |
| (4-trifluoromethanesulfanylphenyl)imidazolidine- | 3-(4-trifluoromethanesulfanylphenyl)imidazolidine-2,4- |
| 2,4-dione | dione |
| 5,5-dimethyl-1-(3-diethylaminopyrid-4-ylmethyl)-3- | 5,5-dimethyl-1-(3-N-cyclohexyl-N-methylaminopyrid- |
| (4-trifluoromethanesulfanylphenyl)imidazolidine- | 4-ylmethyl)-3-(4- |
| 2,4-dione | trifluoromethanesulfanylphenyl)imidazolidine-2,4- |
| | dione |
| 5,5-dimethyl-1-(3-piperidinopyrid-4-ylmethyl)-3-(4- | 5,5-dimethyl-1-(3-morpholinopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[3-(4-methylpiperazino)pyrid-4-yl- | 5,5-dimethyl-1-[3-(1H-pyrazol-1-yl)pyrid-4-ylmethyl]- |
| methyl]-3-(4- | 3-(4-trifluoromethanesulfanylphenyl)imidazolidine-2,4- |
| trifluoromethanesulfanylphenyl)imidazolidine-2,4- | dione |
| dione | |
| 5,5-dimethyl-1-[3-(2-benzoylhydrazino)pyrid-4-yl- | 5,5-dimethyl-1-(3-benzoylaminopyrid-4-ylmethyl)-3- |
| methyl]-3-(4- | (4-trifluoromethanesulfanylphenyl)imidazolidine-2,4- |
| trifluoromethanesulfanylphenyl)imidazolidine-2,4- | dione |
| dione | |
| 5,5-dimethyl-1-[3-(4-chlorobenzoylamino)pyrid-4- | 5,5-dimethyl-1-[3-(3-chlorobenzoylamino)pyrid-4-yl- |
| ylmethyl]-3-(4- | methyl]-3-(4-trifluoromethanesulfanylphenyl)imidazol- |
| trifluoromethanesulfanylphenyl)imidazolidine-2,4- | idine-2,4-dione |
| dione | |
| 5,5-dimethyl-1-[3-(2-chlorobenzoylamino)pyrid-4- | 5,5-dimethyl-1-[3-(4-fluorobenzoylamino)pyrid-4-yl- |
| ylmethyl]-3-(4- | methyl]-3-(4-trifluoromethanesulfanylphenyl)imidazol- |
| trifluoromethanesulfanylphenyl)imidazolidine-2,4- | idine-2,4-dione |
| dione | |
| 5,5-dimethyl-1-[3-(4-methoxybenzoylamino)pyrid- | 5,5-dimethyl-1-(3-acetamidopyrid-4-ylmethyl)-3-(4- |
| 4-ylmethyl]-3-(4- | trifluoromethanesulfanylphenyl)imidazolidine-2,4- |
| trifluoromethanesulfanylphenyl)imidazolidine-2,4- | dione |
| dione | |
| 5,5-dimethyl-1-(3-phenylacetamidopyrid-4- | 5,5-dimethyl-1-[3-(4- |
| ylmethyl)-3-(4- | methylpiperazino)acetamidopyrid-4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-cyclohexanylcarboxamidopyrid- | 5,5-dimethyl-1-[3-(1-methyl-4-piperidinyl)carbonyl- |
| 4-ylmethyl)-3-(4- | aminopyrid-4-ylmethyl]-3-(4-trifluoromethanesulfanyl- |
| trifluoromethanesulfanylphenyl)imidazolidine-2,4- | phenyl)imidazolidine-2,4-dione |
| dione | |
| 5,5-dimethyl-1-[3-(2-furanylcarbonyl)aminopyrid-4- | 5,5-dimethyl-1-(3-ethoxycarbonylaminopyrid-4- |
| ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-isopropoxycarbonylaminopyrid- | 5,5-dimethyl-1-(3-butoxycarbonylaminopyrid-4- |
| 4-ylmethyl)-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[3-(2- | 5,5-dimethyl-1-(3-phenoxycarbonylaminopyrid-4-yl- |
| diethylaminoethoxycarbonylamino)pyrid-4- | methyl)-3-(4-trifluoromethanesulfanylphenyl)imidazol- |
| ylmethyl]-3-(4-trifluoromethanesulfanylphenyl)- | idine-2,4-dione |
| imidazolidine-2,4-dione | |
| 5,5-dimethyl-1-[3-(4- | 5,5-dimethyl-1-(3-anilinocarbonylaminopyrid-4-yl- |
| chloroanilino)carbonylaminopyrid-4-ylmethyl]-3-(4- | methyl)-3-(4-trifluoromethanesulfanylphenyl)imidazol- |
| trifluoromethanesulfanylphenyl)imidazolidine-2,4- | idine-2,4-dione |
| dione | |
| 5,5-dimethyl-1-(3-benzylaminocarbonylaminopyrid- | 5,5-dimethyl-1-[3-(2- |
| 4-ylmethyl)-3-(4- | pyridylamino)carbonylaminopyrid-4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-ureidopyrid-4-ylmethyl)-3-(4- | 5,5-dimethyl-1-(3-benzylaminosulfonylaminopyrid-4- |
| trifluoromethanesulfanylphenyl)imidazolidine-2,4- | ylmethyl)-3-(4- |
| dione | trifluoromethanesulfanylphenyl)imidazolidine-2,4- |
| | dione |
| 5,5-dimethyl-1-(3-methylaminosulfonylaminopyrid- | 5,5-dimethyl-1-(3-aminosulfonylaminopyrid-4- |
| 4-ylmethyl)-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-morpholinosulfonylaminopyrid-4- | 5,5-dimethyl-1-(3-dimethylaminosulfonylaminopyrid- |
| ylmethyl)-3-(4- | 4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-anilinosulfonylaminopyrid-4-yl- | 5,5-dimethyl-1-(3-phenylsulfonylaminopyrid-4- |
| methyl)-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[3-(2-thienyl)sulfonylaminopyrid-4- | 5,5-dimethyl-1-(3-methylsulfonylaminopyrid-4- |
| ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[3-(4- | 5,5-dimethyl-1-(3-benzylsulfonylaminopyrid-4- |
| chlorophenyl)sulfonylaminopyrid-4-ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-hydroxycarbonylpyrid-4- | 5,5-dimethyl-1-(3-ethoxycarbonylpyrid-4-ylmethyl)-3- |
| ylmethyl)-3-(4- | (4-trifluoromethanesulfanylphenyl)imidazolidine-2,4- |
| trifluoromethanesulfanylphenyl)imidazolidine-2,4- | dione |
| dione | |
| 5,5-dimethyl-1-(3-tert-butoxycarbonylpyrid-4- | 5,5-dimethyl-1-(3-phenoxycarbonylpyrid-4-ylmethyl)- |
| ylmethyl)-3-(4- | 3-(4-trifluoromethanesulfanylphenyl)imidazolidine-2,4- |
| trifluoromethanesulfanylphenyl)imidazolidine-2,4- | dione |
| dione | |
| 5,5-dimethyl-1-[3-(4- | 5,5-dimethyl-1-[3-(methylamino)carbonylpyrid-4-yl- |
| methylpiperazinocarbonyl)pyrid-4-ylmethyl]-3-(4- | methyl]-3-(4-trifluoromethanesulfanylphenyl)imidazol- |
| trifluoromethanesulfanylphenyl)imidazolidine-2,4- | idine-2,4-dione |
| dione | |
| 5,5-dimethyl-1-[3-(dimethylamino)carbonylpyrid-4- | 5,5-dimethyl-1-(3-piperidinocarbonylpyrid-4- |
| ylmethyl]-3-(4- | ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-morpholinocarbonylpyrid-4- | 5,5-dimethyl-1-[3-(4-phenylpiperazinocarbonyl)pyrid- |
| ylmethyl)-3-(4- | 4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-phenylaminocarbonylpyrid-4- | 5,5-dimethyl-1-[3-(methylphenylamino)carbonylpyrid- |
| ylmethyl)-3-(4- | 4-ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[3-(4- | 5,5-dimethyl-1-[3-(4- |
| methoxyphenyl)methylaminocarbonylpyrid-4- | chlorophenyl)methylaminocarbonylpyrid-4-ylmethyl]- |
| ylmethyl]-3-(4-trifluoromethanesulfanylphenyl)- | 3-(4-trifluoromethanesulfanylphenyl)imidazolidine-2,4- |
| imidazolidine-2,4-dione | dione |
| 5,5-dimethyl-1-[3-(4- | 5,5-dimethyl-1-(3-aminocarbonylpyrid-4-ylmethyl)-3- |
| morpholinylamino)carbonylpyrid-4-ylmethyl]-3-(4- | (4-trifluoromethanesulfanylphenyl)imidazolidine-2,4- |
| trifluoromethanesulfanylphenyl)imidazolidine-2,4- | dione |
| dione | |
| 5,5-dimethyl-1-(3-phenoxypyrid-4-ylmethyl)-3-(4- | 5,5-dimethyl-1-(3-methoxypyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-(3-ethoxypyrid-4-ylmethyl)-3-(4- | 5,5-dimethyl-1-[3-(1-pyrrolidinyl)ethoxypyrid-4-yl- |
| trifluoromethanesulfanylphenyl)imidazolidine-2,4- | methyl]-3-(4-trifluoromethanesulfanylphenyl)imidazol- |
| dione | idine-2,4-dione |
| 5,5-dimethyl-1-(3-benzyloxypyrid-4-ylmethyl)-3-(4- | 5,5-dimethyl-1-(3-benzoyloxypyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[3-(4,5-dihydro-1H-imidazol-2- | 5,5-dimethyl-1-[3-(1H-imidazol-2-yl)pyrid-4- |
| yl)pyrid-4-ylmethyl]-3-(4- | ylmethyl]-3-(4- |
| trifluoromethanesulfanylphenyl)imidazolidine-2,4- | trifluoromethanesulfanylphenyl)imidazolidine-2,4- |
| dione | dione |
| 5,5-dimethyl-1-[3-(1H-tetrazol-5-yl)pyrid-4- | 5,5-dimethyl-1-(3-amidinopyrid-4-ylmethyl)-3-(4- |
| ylmethyl]-3-(4- | trifluoromethanesulfanylphenyl)imidazolidine-2,4- |
| trifluoromethanesulfanylphenyl)imidazolidine-2,4- | dione |
| dione | |
| 5,5-dimethyl-1-(3-dimethylaminomethylpyrid-4-yl- | 5,5-dimethyl-1-(3-aminomethylpyrid-4-ylmethyl)-3-(4- |
| methyl)-3-(4- | trifluoromethanesulfanylphenyl)imidazolidine-2,4- |
| trifluoromethanesulfanylphenyl)imidazolidine-2,4- | dione |
| dione | |
| 4-(2-methylpyridin)-4-ylmethyl-6-(4- | 4-(2-ethylpyridin)-4-ylmethyl-6-(4-trifluoromethane- |
| trifluoromethanesulfanylphenyl)-4,6- | sulfanylphenyl)-4,6-diazaspiro[2.4]heptane-5,7-dione |
| diazaspiro[2.4]heptane-5,7-dione | |
| 4-(2-isopropylpyridin)-4-ylmethyl-6-(4-trifluoro- | 4-(2-tert-butylpyridin)-4-ylmethyl-6-(4-trifluoro- |
| methanesulfanylphenyl)-4,6-diazaspiro[2.4]heptane- | methane sulfanylphenyl)-4,6-diazaspiro[2.4]heptane- |
| 5,7-dione | 5,7-dione |
| 4-(2-isobutylpyridin)-4-ylmethyl-6-(4- | 4-(2-cyclohexylpyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(2-phenylpyridin)-4-ylmethyl-6-(4-trifluoro- | 4-[2-(4-chlorophenyl)pyridin]-4-ylmethyl-6-(4- |
| methanesulfanylphenyl)-4,6-diazaspiro[2.4]heptane- | trifluoromethanesulfanylphenyl)-4,6-diazaspiro[2.4]- |
| 5,7-dione | heptane-5,7-dione |
| 4-[2-(3-chlorophenyl)pyridin]-4-ylmethyl-6-(4- | 4-[2-(2-chlorophenyl)pyridin]-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(4-fluorophenyl)pyridin]-4-ylmethyl-6-(4- | 4-[2-(4-methoxyphenyl)pyridin]-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(2-benzylpyridin)-4-ylmethyl-6-(4-trifluoro- | 4-(2,2′-bipyridin)-4-ylmethyl-6-(4-trifluoromethane- |
| methanesulfanylphenyl)-4,6-diazaspiro[2.4]heptane- | sulfanylphenyl)-4,6-diazaspiro[2.4]heptane-5,7-dione |
| 5,7-dione | |
| 4-(2-hydroxymethylpyridin)-4-ylmethyl-6-(4- | 4-(2-chloropyridin)-4-ylmethyl-6-(4-trifluoromethane- |
| trifluoromethanesulfanylphenyl)-4,6- | sulfanylphenyl)-4,6-diazaspiro[2.4]heptane-5,7-dione |
| diazaspiro[2.4]heptane-5,7-dione | |
| 4-(2-fluoropyridin)-4-ylmethyl-6-(4-trifluoro- | 4-(2-bromopyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(2-aminopyridin)-4-ylmethyl-6-(4-trifluoro- | 4-(2-methylaminopyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(2-ethylaminopyridin)-4-ylmethyl-6-(4-trifluoro- | 4-(2-isopropylaminopyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(2-phenylaminopyridin)-4-ylmethyl-6-(4-trifluoro- | 4-(2-cyclohexylaminopyridin)-4-ylmethyl-6-(4- |
| methanesulfanylphenyl)-4,6-diazaspiro[2.4]heptane- | trifluoromethanesulfanylphenyl)-4,6- |
| 5,7-dione | diazaspiro[2.4]heptane-5,7-dione |
| 4-(2-diethylaminopyridin)-4-ylmethyl-6-(4-trifluoro- | 4-(2-N-cyclohexyl-N-methylaminopyridin)-4-ylmethyl- |
| methanesulfanylphenyl)-4,6-diazaspiro[2.4]heptane- | 6-(4-trifluoromethanesulfanylphenyl)-4,6- |
| 5,7-dione | diazaspiro[2.4]heptane-5,7-dione |
| 4-(2-piperidinopyridin)-4-ylmethyl-6-(4-trifluoro- | 4-(2-morpholinopyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-[2-(4-methylpiperazino)pyridin]-4-ylmethyl-6-(4- | 4-[2-(1H-pyrazol-1-yl)pyridin]-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(2-benzoylhydrazino)pyridin]-4-ylmethyl-6-(4- | 4-(2-benzoylaminopyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-[2-(4-chlorobenzoylamino)pyridin]-4-ylmethyl-6- | 4-[2-(3-chlorobenzoylamino)pyridin]-4-ylmethyl-6-(4- |
| (4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(2-chlorobenzoylamino)pyridin]-4-ylmethyl-6- | 4-[2-(4-fluorobenzoylamino)pyridin]-4-ylmethyl-6-(4- |
| (4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(4-methoxybenzoylamino)pyridin]-4-ylmethyl- | 4-(2-acetamidopyridin)-4-ylmethyl-6-(4-trifluoro- |
| 6-(4-trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(2-phenylacetamidopyridin)-4-ylmethyl-6-(4- | 4-[2-(4-methylpiperazino)acetamidopyridin]-4- |
| trifluoromethanesulfanylphenyl)-4,6- | ylmethyl-6-(4-trifluoromethanesulfanylphenyl)-4,6- |
| diazaspiro[2.4]heptane-5,7-dione | diazaspiro[2.4]heptane-5,7-dione |
| 4-(2-cyclohexanylcarboxamidopyridin)-4-ylmethyl- | 4-[2-(1-methyl-4-piperidinyl)carbonylaminopyridin]-4- |
| 6-(4-trifluoromethanesulfanylphenyl)-4,6- | ylmethyl-6-(4-trifluoromethanesulfanylphenyl)-4,6- |
| diazaspiro[2.4]heptane-5,7-dione | diazaspiro[2.4]heptane-5,7-dione |
| 4-[2-(2-furanylcarbonyl)aminopyridin]-4-ylmethyl- | 4-(2-ethoxycarbonylaminopyridin)-4-ylmethyl-6-(4- |
| 6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(2-isopropoxycarbonylaminopyridin)-4-ylmethyl- | 4-(2-butoxycarbonylaminopyridin)-4-ylmethyl-6-(4- |
| 6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(2-diethylaminoethoxycarbonylamino)pyridin]- | 4-(2-phenoxycarbonylaminopyridin)-4-ylmethyl-6-(4- |
| 4-ylmethyl-6-(4-trifluoromethanesulfanylphenyl)- | trifluoromethanesulfanylphenyl)-4,6-diazaspiro[2.4]- |
| 4,6-diazaspiro[2.4]heptane-5,7-dione | heptane-5,7-dione |
| 4-[2-(4-chloroanilino)carbonylaminopyridin]-4-yl- | 4-(2-anilinocarbonylaminopyridin)-4-ylmethyl-6-(4- |
| methyl)-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(2-benzylaminocarbonylaminopyridin)-4- | 4-[2-(2-pyridylamino)carbonylaminopyridin]-4- |
| ylmethyl-6-(4-trifluoromethanesulfanylphenyl)-4,6- | ylmethyl-6-(4-trifluoromethanesulfanylphenyl)-4,6- |
| diazaspiro[2.4]heptane-5,7-dione | diazaspiro[2.4]heptane-5,7-dione |
| 4-(2-ureidopyridin)-4-ylmethyl-6-(4-trifluoro- | 4-(2-benzylaminosulfonylaminopyridin)-4-ylmethyl-6- |
| methanesulfanylphenyl)-4,6-diazaspiro[2.4]heptane- | (4-trifluoromethanesulfanylphenyl)-4,6-diazaspiro[2.4]- |
| 5,7-dione | heptane-5,7-dione |
| 4-(2-methylaminosulfonylaminopyridin)-4- | 4-(2-aminosulfonylaminopyridin)-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(2-morpholinosulfonylaminopyridin)-4-ylmethyl- | 4-(2-dimethylaminosulfonylaminopyridin)-4-ylmethyl- |
| 6-(4-trifluoromethanesulfanylphenyl)-4,6- | 6-(4-trifluoromethanesulfanylphenyl)-4,6- |
| diazaspiro[2.4]heptane-5,7-dione | diazaspiro[2.4]heptane-5,7-dione |
| 4-(2-anilinosulfonylaminopyridin)-4-ylmethyl-6-(4- | 4-(2-phenylsulfonylaminopyridin)-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(2-thienyl)sulfonylaminopyridin]-4-ylmethyl-6- | 4-(2-methylsulfonylaminopyridin)-4-ylmethyl-6-(4- |
| (4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(4-chlorophenyl)sulfonylaminopyridin]-4- | 4-(2-benzylsulfonylaminopyridin)-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(2-hydroxycarbonylpyridin)-4-ylmethyl-6-(4- | 4-(2-ethoxycarbonylpyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(2-tert-butoxycarbonylpyridin)-4-ylmethyl-6-(4- | 4-(2-phenoxycarbonylpyridin)-4-ylmethyl-6-(4- |
| trifluoromethanesulfanylphenyl)-4,6-diazaspiro[2.4] | trifluoromethanesulfanylphenyl)-4,6- |
| heptane-5,7-dione | diazaspiro[2.4]heptane-5,7-dione |
| 4-[2-(4-methylpiperazinocarbonyl)pyridin]-4- | 4-[2-(methylamino)carbonylpyridin]-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(dimethylamino)carbonylpyridin]-4-ylmethyl- | 4-(2-piperidinocarbonylpyridin)-4-ylmethyl-6-(4- |
| 6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(2-morpholinocarbonylpyridin)-4-ylmethyl-6-(4- | 4-[2-(4-phenylpiperazinocarbonyl)pyridin]-4-ylmethyl- |
| trifluoromethanesulfanylphenyl)-4,6- | 6-(4-trifluoromethanesulfanylphenyl)-4,6- |
| diazaspiro[2.4]heptane-5,7-dione | diazaspiro[2.4]heptane-5,7-dione |
| 4-(2-phenylaminocarbonylpyridin)-4-ylmethyl-6-(4- | 4-[2-(methylphenylamino)carbonylpyridin]-4-ylmethyl- |
| trifluoromethanesulfanylphenyl)-4,6- | 6-(4-trifluoromethanesulfanylphenyl)-4,6- |
| diazaspiro[2.4]heptane-5,7-dione | diazaspiro[2.4]heptane-5,7-dione |
| 4-[2-(4- | 4-[2-(4-chlorophenyl)methylaminocarbonylpyridin]-4- |
| methoxyphenyl)methylaminocarbonylpyridin]-4- | ylmethyl-6-(4-trifluoromethanesulfanylphenyl)-4,6- |
| ylmethyl-6-(4-trifluoromethanesulfanylphenyl)-4,6- | diazaspiro[2.4]heptane-5,7-dione |
| diazaspiro[2.4]heptane-5,7-dione | |
| 4-[2-(4-morpholinylamino)carbonylpyridin]-4- | 4-(2-aminocarbonylpyridin)-4-ylmethyl-6-(4-trifluoro- |
| ylmethyl-6-(4-trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(2-phenoxypyridin)-4-ylmethyl-6-(4-trifluoro- | 4-(2-methoxypyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(2-ethoxypyridin)-4-ylmethyl-6-(4-trifluoro- | 4-[2-(1-pyrrolidinyl)ethoxypyridin]-4-ylmethyl-6-(4- |
| methanesulfanylphenyl)-4,6-diazaspiro[2.4]heptane- | trifluoromethanesulfanylphenyl)-4,6-diazaspiro[2.4]- |
| 5,7-dione | heptane-5,7-dione |
| 4-(2-benzyloxypyridin)-4-ylmethyl-6-(4-trifluoro- | 4-(2-benzoyloxypyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-[2-(4,5-dihydro-1H-imidazol-2-yl)pyridin]-4- | 4-[2-(1H-imidazol-2-yl)pyridin]-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[2-(1H-tetrazol-5-yl)pyridin]-4-ylmethyl-6-(4- | 4-(2-amidinopyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(2-dimethylaminomethylpyridin)-4-ylmethyl-6-(4- | 4-(2-aminomethylpyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(3-methylpyridin)-4-ylmethyl-6-(4-trifluoro- | 4-(3-ethylpyridin)-4-ylmethyl-6-(4-trifluoromethane- |
| methanesulfanylphenyl)-4,6-diazaspiro[2.4]heptane- | sulfanylphenyl)-4,6-diazaspiro[2.4]heptane-5,7-dione |
| 5,7-dione | |
| 4-(3-isopropylpyridin)-4-ylmethyl-6-(4-trifluoro- | 4-(3-tert-butylpyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(3-isobutylpyridin)-4-ylmethyl-6-(4-trifluoro- | 4-(3-cyclohexylpyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(3-phenylpyridin)-4-ylmethyl-6-(4-trifluoro- | 4-[3-(4-chlorophenyl)pyridin]-4-ylmethyl-6-(4- |
| methanesulfanylphenyl)-4,6-diazaspiro[2.4]heptane- | trifluoromethanesulfanylphenyl)-4,6-diazaspiro[2.4]- |
| 5,7-dione | heptane-5,7-dione |
| 4-[3-(3-chlorophenyl)pyridin]-4-ylmethyl-6-(4- | 4-[3-(2-chlorophenyl)pyridin]-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(4-fluorophenyl)pyridin]-4-ylmethyl-6-(4- | 4-[3-(4-methoxyphenyl)pyridin]-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(3-benzylpyridin)-4-ylmethyl-6-(4-trifluoro- | 4-(3,2′-bipyridin)-4-ylmethyl-6-(4-trifluoromethane- |
| methanesulfanylphenyl)-4,6-diazaspiro[2.4]heptane- | sulfanylphenyl)-4,6-diazaspiro[2.4]heptane-5,7-dione |
| 5,7-dione | |
| 4-(3-hydroxymethylpyridin)-4-ylmethyl-6-(4- | 4-(3-chloropyridin)-4-ylmethyl-6-(4-trifluoromethane- |
| trifluoromethanesulfanylphenyl)-4,6- | sulfanylphenyl)-4,6-diazaspiro[2.4]heptane-5,7-dione |
| diazaspiro[2.4]heptane-5,7-dione | |
| 4-(3-fluoropyridin)-4-ylmethyl-6-(4-trifluoro- | 4-(3-bromopyridin)-4-ylmethyl-6-(4-trifluoromethane- |
| methanesulfanylphenyl)-4,6-diazaspiro[2.4]heptane- | sulfanylphenyl)-4,6-diazaspiro[2.4]heptane-5,7-dione |
| 5,7-dione | |
| 4-(3-aminopyridin)-4-ylmethyl-6-(4-trifluoro- | 4-(3-methylaminopyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(3-ethylaminopyridin)-4-ylmethyl-6-(4-trifluoro- | 4-(3-isopropylaminopyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(3-phenylaminopyridin)-4-ylmethyl-6-(4-trifluoro- | 4-(3-cyclohexylaminopyridin)-4-ylmethyl-6-(4- |
| methanesulfanylphenyl)-4,6-diazaspiro[2.4]heptane- | trifluoromethanesulfanylphenyl)-4,6- |
| 5,7-dione | diazaspiro[2.4]heptane-5,7-dione |
| 4-(3-diethylaminopyridin)-4-ylmethyl-6-(4-trifluoro- | 4-(3-N-cyclohexyl-N-methylaminopyridin)-4-ylmethyl- |
| methanesulfanylphenyl)-4,6-diazaspiro[2.4]heptane- | 6-(4-trifluoromethanesulfanylphenyl)-4,6- |
| 5,7-dione | diazaspiro[2.4]heptane-5,7-dione |
| 4-(3-piperidinopyridin)-4-ylmethyl-6-(4-trifluoro- | 4-(3-morpholinopyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-[3-(4-methylpiperazino)pyridin]-4-ylmethyl-6-(4- | 4-[3-(1H-pyrazol-1-yl)pyridin]-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(2-benzoylhydrazino)pyridin]-4-ylmethyl-6-(4- | 4-(3-benzoylaminopyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-[3-(4-chlorobenzoylamino)pyridin]-4-ylmethyl-6- | 4-[3-(3-chlorobenzoylamino)pyridin]-4-ylmethyl-6-(4- |
| (4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(2-chlorobenzoylamino)pyridin]-4-ylmethyl-6- | 4-[3-(4-fluorobenzoylamino)pyridin]-4-ylmethyl-6-(4- |
| (4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(4-methoxybenzoylamino)pyridin]-4-ylmethyl- | 4-(3-acetamidopyridin)-4-ylmethyl-6-(4-trifluoro- |
| 6-(4-trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(3-phenylacetamidopyridin)-4-ylmethyl-6-(4- | 4-[3-(4-methylpiperazino)acetamidopyridin]-4- |
| trifluoromethanesulfanylphenyl)-4,6- | ylmethyl-6-(4-trifluoromethanesulfanylphenyl)-4,6- |
| diazaspiro[2.4]heptane-5,7-dione | diazaspiro[2.4]heptane-5,7-dione |
| 4-(3-cyclohexanylcarboxamidopyridin)-4-ylmethyl- | 4-[3-(1-methyl-4-piperidinyl)carbonylaminopyridin]-4- |
| 6-(4-trifluoromethanesulfanylphenyl)-4,6- | ylmethyl-6-(4-trifluoromethanesulfanylphenyl)-4,6- |
| diazaspiro[2.4]heptane-5,7-dione | diazaspiro[2.4]heptane-5,7-dione |
| 4-[3-(2-furanylcarbonyl)aminopyridin]-4-ylmethyl- | 4-(3-ethoxycarbonylaminopyridin)-4-ylmethyl-6-(4- |
| 6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(3-isopropoxycarbonylaminopyridin)-4-ylmethyl- | 4-(3-butoxycarbonylaminopyridin)-4-ylmethyl-6-(4- |
| 6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(2-diethylaminoethoxycarbonylamino)pyridin]- | 4-(3-phenoxycarbonylaminopyridin)-4-ylmethyl-6-(4- |
| 4-ylmethyl-6-(4-trifluoromethanesulfanylphenyl)- | trifluoromethanesulfanylphenyl)-4,6-diazaspiro[2.4]- |
| 4,6-diazaspiro[2.4]heptane-5,7-dione | heptane-5,7-dione |
| 4-[3-(4-chloroanilino)carbonylaminopyridin]-4-yl- | 4-(3-anilinocarbonylaminopyridin)-4-ylmethyl-6-(4- |
| methyl)-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(3-benzylaminocarbonylaminopyridin)-4- | 4-[3-(2-pyridylamino)carbonylaminopyridin]-4- |
| ylmethyl-6-(4-trifluoromethanesulfanylphenyl)-4,6- | ylmethyl-6-(4-trifluoromethanesulfanylphenyl)-4,6- |
| diazaspiro[2.4]heptane-5,7-dione | diazaspiro[2.4]heptane-5,7-dione |
| 4-(3-ureidopyridin)-4-ylmethyl-6-(4-trifluoro- | 4-(3-benzylaminosulfonylaminopyridin)-4-ylmethyl-6- |
| methanesulfanylphenyl)-4,6-diazaspiro[2.4]heptane- | (4-trifluoromethanesulfanylphenyl)-4,6-diazaspiro[2.4]- |
| 5,7-dione | heptane-5,7-dione |
| 4-(3-methylaminosulfonylaminopyridin)-4- | 4-(3-aminosulfonylaminopyridin)-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(3-morpholinosulfonylaminopyridin)-4-ylmethyl- | 4-(3-dimethylaminosulfonylaminopyridin)-4-ylmethyl- |
| 6-(4-trifluoromethanesulfanylphenyl)-4,6- | 6-(4-trifluoromethanesulfanylphenyl)-4,6- |
| diazaspiro[2.4]heptane-5,7-dione | diazaspiro[2.4]heptane-5,7-dione |
| 4-(3-anilinosulfonylaminopyridin)-4-ylmethyl-6-(4- | 4-(3-phenylsulfonylaminopyridin)-4-ylmethyl-6-(4- |
| trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(2-thienyl)sulfonylaminopyridin]-4-ylmethyl-6- | 4-(3-methylsulfonylaminopyridin)-4-ylmethyl-6-(4- |
| (4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(4-chlorophenyl)sulfonylaminopyridin]-4- | 4-(3-benzylsulfonylaminopyridin)-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(3-hydroxycarbonylpyridin)-4-ylmethyl-6-(4- | 4-(3-ethoxycarbonylpyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(3-tert-butoxycarbonylpyridin)-4-ylmethyl-6-(4- | 4-(3-phenoxycarbonylpyridin)-4-ylmethyl-6-(4- |
| trifluoromethanesulfanylphenyl)-4,6- | trifluoromethanesulfanylphenyl)-4,6- |
| diazaspiro[2.4]heptane-5,7-dione | diazaspiro[2.4]heptane-5,7-dione |
| 4-[3-(4-methylpiperazinocarbonyl)pyridin]-4- | 4-[3-(methylamino)carbonylpyridin]-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(dimethylamino)carbonylpyridin]-4-ylmethyl- | 4-(3-piperidinocarbonylpyridin)-4-ylmethyl-6-(4- |
| 6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-(3-morpholinocarbonylpyridin)-4-ylmethyl-6-(4- | 4-[3-(4-phenylpiperazinocarbonyl)pyridin]-4-ylmethyl- |
| trifluoromethanesulfanylphenyl)-4,6- | 6-(4-trifluoromethanesulfanylphenyl)-4,6- |
| diazaspiro[2.4]heptane-5,7-dione | diazaspiro[2.4]heptane-5,7-dione |
| 4-(3-phenylaminocarbonylpyridin)-4-ylmethyl-6-(4- | 4-[3-(methylphenylamino)carbonylpyridin]-4-ylmethyl- |
| trifluoromethanesulfanylphenyl)-4,6- | 6-(4-trifluoromethanesulfanylphenyl)-4,6- |
| diazaspiro[2.4]heptane-5,7-dione | diazaspiro[2.4]heptane-5,7-dione |
| 4-[3-(4- | 4-[3-(4-chlorophenyl)methylaminocarbonylpyridin]-4- |
| methoxyphenyl)methylaminocarbonylpyridin]-4- | ylmethyl-6-(4-trifluoromethanesulfanylphenyl)-4,6- |
| ylmethyl-6-(4-trifluoromethanesulfanylphenyl)-4,6- | diazaspiro[2.4]heptane-5,7-dione |
| diazaspiro[2.4]heptane-5,7-dione | |
| 4-[3-(4-morpholinylamino)carbonylpyridin]-4- | 4-(3-aminocarbonylpyridin)-4-ylmethyl-6-(4-trifluoro- |
| ylmethyl-6-(4-trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(3-phenoxypyridin)-4-ylmethyl-6-(4-trifluoro- | 4-(3-methoxypyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-(3-ethoxypyridin)-4-ylmethyl-6-(4-trifluoro- | 4-[3-(1-pyrrolidinyl)ethoxypyridin]-4-ylmethyl-6-(4- |
| methanesulfanylphenyl)-4,6-diazaspiro[2.4]heptane- | trifluoromethanesulfanylphenyl)-4,6-diazaspiro[2.4]- |
| 5,7-dione | heptane-5,7-dione |
| 4-(3-benzyloxypyridin)-4-ylmethyl-6-(4-trifluoro- | 4-(3-benzoyloxypyridin)-4-ylmethyl-6-(4-trifluoro- |
| methanesulfanylphenyl)-4,6-diazaspiro[2.4]heptane- | methanesulfanylphenyl)-4,6-diazaspiro[2.4]heptane- |
| 5,7-dione | 5,7-dione |
| 4-[3-(4,5-dihydro-1H-imidazol-2-yl)pyridin]-4- | 4-[3-(1H-imidazol-2-yl)pyridin]-4-ylmethyl-6-(4- |
| ylmethyl-6-(4-trifluoromethanesulfanylphenyl)-4,6- | trifluoromethanesulfanylphenyl)-4,6-diazaspiro[2.4]- |
| diazaspiro[2.4]heptane-5,7-dione | heptane-5,7-dione |
| 4-[3-(1H-tetrazol-5-yl)pyridin]-4-ylmethyl-6-(4- | 4-(3-amidinopyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 4-(3-dimethylaminomethylpyridin)-4-ylmethyl-6-(4- | 4-(3-aminomethylpyridin)-4-ylmethyl-6-(4-trifluoro- |
| trifluoromethanesulfanylphenyl)-4,6- | methanesulfanylphenyl)-4,6-diazaspiro[2.4]heptane- |
| diazaspiro[2.4]heptane-5,7-dione | 5,7-dione |
| 5-methyl-1-(2-methylpyrid-4-ylmethyl)-3-(4- | 5-methyl-1-(2-ethylpyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-(2-isopropylpyrid-4-ylmethyl)-3-(4- | 5-methyl-1-(2-tert-butylpyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5-methyl-1-(2-isobutylpyrid-4-ylmethyl)-3-(4- | 5-methyl-1-(2-cyclohexylpyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5-methyl-1-(2-phenylpyrid-4-ylmethyl)-3-(4- | 5-methyl-1-[2-(4-chlorophenyl)pyrid-4-ylmethyl]-3-(4- |
| trifluoromethanesulfanylphenyl)imidazolidine-2,4- | trifluoromethanesulfanylphenyl)imidazolidine-2,4- |
| dione | dione |
| 5-methyl-1-[2-(3-chlorophenyl)pyrid-4-ylmethyl]-3- | 5-methyl-1-[2-(2-chlorophenyl)pyrid-4-ylmethyl]-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-[2-(4-methoxyphenyl)pyrid-4-ylmethyl]- | 5-methyl-1-(2-benzylpyrid-4-ylmethyl)-3-(4-trifluoro- |
| 3-(4-trifluoromethanesulfanylphenyl)imidazolidine- | methanesulfanylphenyl)imidazolidine-2,4-dione |
| 2,4-dione | |
| 5-methyl-1-(2,2′-bipyrid-4-ylmethyl)-3-(4-trifluoro- | 5-methyl-1-(2-hydroxymethylpyrid-4-ylmethyl)-3-(4- |
| methanesulfanylphenyl)imidazolidine-2,4-dione | trifluoromethanesulfanylphenyl)imidazolidine-2,4- |
| | dione |
| 5-methyl-1-(2-chloropyrid-4-ylmethyl)-3-(4- | 5-methyl-1-(2-fluoropyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-(2-bromopyrid-4-ylmethyl)-3-(4- | 5-methyl-1-(2-aminopyrid-4-ylmethyl)-3-(4-trifluoro- |
| trifluoromethanesulfanylphenyl)imidazolidine-2,4- | methanesulfanylphenyl)imidazolidine-2,4-dione |
| dione; | |
| 5-methyl-1-(2-methylaminopyrid-4-ylmethyl)-3-(4- | 5-methyl-1-(2-ethylaminopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5-methyl-1-(2-isopropylaminopyrid-4-ylmethyl)-3- | 5-methyl-1-(2-phenylaminopyrid-4-ylmethyl)-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-(2-cyclohexylaminopyrid-4-ylmethyl)-3- | 5-methyl-1-(2-diethylaminopyrid-4-ylmethyl)-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-(2-N-cyclohexyl-N-methylaminopyrid- | 5-methyl-1-(2-piperidinopyrid-4-ylmethyl)-3-(4- |
| 4-ylmethyl)-3-(4-trifluoro- | trifluoromethanesulfanylphenyl)imidazolidine-2,4- |
| methanesulfanylphenyl)imidazolidine-2,4-dione | dione |
| 5-methyl-1-(2-morpholinopyrid-4-ylmethyl)-3-(4- | 5-methyl-1-[2-(4-methylpiperazino)pyrid-4-ylmethyl]- |
| trifluoromethanesulfanylphenyl)imidazolidine-2,4- | 3-(4-trifluoromethanesulfanylphenyl)imidazolidine-2,4- |
| dione | dione |
| 5-methyl-1-[2-(1H-pyrazol-1-yl)pyrid-4-ylmethyl]- | 5-methyl-1-[2-(2-benzoylhydrazino)pyrid-4-ylmethyl]- |
| 3-(4-trifluoromethanesulfanylphenyl)imidazolidine- | 3-(4-trifluoromethanesulfanylphenyl)imidazolidine-2,4- |
| 2,4-dione | dione |
| 5-methyl-1-(2-benzoylaminopyrid-4-ylmethyl)-3-(4- | 5-methyl-1-[2-(4-chlorobenzoylamino)pyrid-4- |
| trifluoromethanesulfanylphenyl)imidazolidine-2,4- | ylmethyl]-3-(4-trifluoro- |
| dione | methanesulfanylphenyl)imidazolidine-2,4-dione |
| 5-methyl-1-[2-(3-chlorobenzoylamino)pyrid-4- | 5-methyl-1-[2-(2-chlorobenzoylamino)pyrid-4- |
| ylmethyl]-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-[2-(4-fluorobenzoylamino)pyrid-4- | 5-methyl-1-[2-(4-methoxybenzoylamino)pyrid-4- |
| ylmethyl]-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(2-acetamidopyrid-4-ylmethyl)-3-(4- | 5-methyl-1-(2-phenylacetamidopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5-methyl-1-[2-(4-methylpiperazino)acetamidopyrid- | 5-methyl-1-(2-cyclohexanylcarboxamidopyrid-4- |
| 4-ylmethyl]-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-[2-(1-methyl-4- | 5-methyl-1-[2-(2-furanylcarbonyl)aminopyrid-4- |
| piperidinyl)carbonylaminopyrid-4-ylmethyl]-3-(4- | ylmethyl]-3-(4-trifluoro- |
| trifluoromethanesulfanylphenyl)imidazolidine-2,4- | methanesulfanylphenyl)imidazolidine-2,4-dione |
| dione | |
| 5-methyl-1-(2-ethoxycarbonylaminopyrid-4- | 5-methyl-1-(2-isopropoxycarbonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-(2-butoxycarbonylaminopyrid-4- | 5-methyl-1-[2-(2- |
| ylmethyl)-3-(4-trifluoro- | diethylaminoethoxycarbonylamino)pyrid-4-ylmethyl]- |
| methanesulfanylphenyl)imidazolidine-2,4-dione | 3-(4-trifluoromethanesulfanylphenyl)imidazolidine-2,4- |
| | dione |
| 5-methyl-1-(2-phenoxycarbonylaminopyrid-4- | 5-methyl-1-[2-(4-chloroanilino)carbonylaminopyrid-4- |
| ylmethyl)-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(2-anilinocarbonylaminopyrid-4- | 5-methyl-1-(2-benzylaminocarbonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-[2-(2- | 5-methyl-1-(2-ureidopyrid-4-ylmethyl)-3-(4-trifluoro- |
| pyridylamino)carbonylaminopyrid-4-ylmethyl]-3-(4- | methanesulfanylphenyl)imidazolidine-2,4-dione |
| trifluoromethanesulfanylphenyl)imidazolidine-2,4- | |
| dione | |
| 5-methyl-1-(2-benzylaminosulfonylaminopyrid-4- | 5-methyl-1-(2-methylaminosulfonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-(2-aminosulfonylaminopyrid-4- | 5-methyl-1-(2-morpholinosulfonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-(2-dimethylaminosulfonylaminopyrid-4- | 5-methyl-1-(2-anilinosulfonylaminopyrid-4-ylmethyl)- |
| ylmethyl)-3-(4-trifluoro- | 3-(4-trifluoromethanesulfanylphenyl)imidazolidine-2,4- |
| methanesulfanylphenyl)imidazolidine-2,4-dione | dione |
| 5-methyl-1-(2-phenylsulfonylaminopyrid-4- | 5-methyl-1-[2-(2-thienyl)sulfonylaminopyrid-4- |
| ylmethyl)-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(2-methylsulfonylaminopyrid-4- | 5-methyl-1-[2-(4-chlorophenyl)sulfonylaminopyrid-4- |
| ylmethyl)-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(2-benzylsulfonylaminopyrid-4- | 5-methyl-1-(2-hydroxycarbonylpyrid-4-ylmethyl)-3-(4- |
| ylmethyl)-3-(4-trifluoro- | trifluoromethanesulfanylphenyl)imidazolidine-2,4- |
| methanesulfanylphenyl)imidazolidine-2,4-dione | dione |
| 5-methyl-1-(2-ethoxycarbonylpyrid-4-ylmethyl)-3- | 5-methyl-1-(2-tert-butoxycarbonylpyrid-4-ylmethyl)-3- |
| (4-trifluoromethanesulfanylphenyl)imidazolidine- | (4-trifluoromethanesulfanylphenyl)imidazolidine-2,4- |
| 2,4-dione | dione |
| 5-methyl-1-(2-phenoxycarbonylpyrid-4-ylmethyl)-3- | 5-methyl-1-[2-(4-methylpiperazinocarbonyl)pyrid-4- |
| (4-trifluoromethanesulfanylphenyl)imidazolidine- | ylmethyl]-3-(4-trifluoro- |
| 2,4-dione | methanesulfanylphenyl)imidazolidine-2,4-dione |
| 5-methyl-1-[2-(methylamino)carbonylpyrid-4- | 5-methyl-1-[2-(dimethylamino)carbonylpyrid-4- |
| ylmethyl]-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(2-piperidinocarbonylpyrid-4-ylmethyl)- | 5-methyl-1-(2-morpholinocarbonylpyrid-4-ylmethyl)-3- |
| 3-(4-trifluoromethanesulfanylphenyl)imidazolidine- | (4-trifluoromethanesulfanylphenyl)imidazolidine-2,4- |
| 2,4-dione | dione |
| 5-methyl-1-[2-(4-phenylpiperazinocarbonyl)pyrid-4- | 5-methyl-1-(2-phenylaminocarbonylpyrid-4-ylmethyl)- |
| ylmethyl]-3-(4-trifluoro- | 3-(4-trifluoromethanesulfanylphenyl)imidazolidine-2,4- |
| methanesulfanylphenyl)imidazolidine-2,4-dione | dione |
| 5-methyl-1-[2-(methylphenylamino)carbonylpyrid- | 5-methyl-1-[2-(4- |
| 4-ylmethyl]-3-(4-trifluoro- | methoxyphenyl)methylaminocarbonylpyrid-4- |
| methanesulfanylphenyl)imidazolidine-2,4-dione | ylmethyl]-3-(4-trifluoro- |
| | methanesulfanylphenyl)imidazolidine-2,4-dione |
| 5-methyl-1-[2-(4- | 5-methyl-1-[2-(4-morpholinylamino)carbonylpyrid-4- |
| chlorophenyl)methylaminocarbonylpyrid-4- | ylmethyl]-3-(4-trifluoro- |
| ylmethyl]-3-(4-trifluoro- | methanesulfanylphenyl)imidazolidine-2,4-dione |
| methanesulfanylphenyl)imidazolidine-2,4-dione | |
| 5-methyl-1-(2-aminocarbonylpyrid-4-ylmethyl)-3- | 5-methyl-1-(2-phenoxypyrid-4-ylmethyl)-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-(2-methoxypyrid-4-ylmethyl)-3-(4- | 5-methyl-1-(2-ethoxypyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-[2-(1-pyrrolidinyl)ethoxypyrid-4- | 5-methyl-1-(2-benzyloxypyrid-4-ylmethyl)-3-(4- |
| ylmethyl]-3-(4-trifluoro- | trifluoromethanesulfanylphenyl)imidazolidine-2,4- |
| methanesulfanylphenyl)imidazolidine-2,4-dione | dione |
| 5-methyl-1-(2-benzoyloxypyrid-4-ylmethyl)-3-(4- | 5-methyl-1-[2-(4,5-dihydro-1H-imidazol-2-yl)pyrid-4- |
| trifluoromethanesulfanylphenyl)imidazolidine-2,4- | ylmethyl]-3-(4-trifluoro- |
| dione | methanesulfanylphenyl)imidazolidine-2,4-dione |
| 5-methyl-1-[2-(1H-imidazol-2-yl)pyrid-4-ylmethyl]- | 5-methyl-1-[2-(1H-tetrazol-5-yl)pyrid-4-ylmethyl]-3- |
| 3-(4-trifluoromethanesulfanylphenyl)imidazolidine- | (4-trifluoromethanesulfanylphenyl)imidazolidine-2,4- |
| 2,4-dione | dione |
| 5-methyl-1-(2-amidinopyrid-4-ylmethyl)-3-(4- | 5-methyl-1-(2-dimethylaminomethylpyrid-4-ylmethyl)- |
| trifluoromethanesulfanylphenyl)imidazolidine-2,4- | 3-(4-trifluoromethanesulfanylphenyl)imidazolidine-2,4- |
| dione | dione |
| 5-methyl-1-(2-aminomethylpyrid-4-ylmethyl)-3-(4- | 5-methyl-1-(3-methylpyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-(3-ethylpyrid-4-ylmethyl)-3-(4-trifluoro- | 5-methyl-1-(3-isopropylpyrid-4-ylmethyl)-3-(4- |
| methanesulfanylphenyl)imidazolidine-2,4-dione | trifluoromethanesulfanylphenyl)imidazolidine-2,4- |
| | dione |
| 5-methyl-1-(3-tert-butylpyrid-4-ylmethyl)-3-(4- | 5-methyl-1-(3-isobutylpyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-(3-cyclohexylpyrid-4-ylmethyl)-3-(4- | 5-methyl-1-(3-phenylpyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-[3-(4-chlorophenyl)pyrid-4-ylmethyl]-3- | 5-methyl-1-[3-(3-chlorophenyl)pyrid-4-ylmethyl]-3-(4- |
| (4-trifluormethanesulfanylphenyl)imidazolidine-2,4- | trifluoromethanesulfanylphenyl)imidazolidine-2,4- |
| dione | dione |
| 5-methyl-1-[3-(2-chlorophenyl)pyrid-4-ylmethyl]-3- | 5-methyl-1-[3-(4-fluorophenyl)pyrid-4-ylmethyl]-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-[3-(4-methoxyphenyl)pyrid-4-ylmethyl]- | 5-methyl-1-(3-benzylpyrid-4-ylmethyl)-3-(4-trifluoro- |
| 3-(4-trifluoromethanesulfanylphenyl)imidazolidine- | methanesulfanylphenyl)imidazolidine-2,4-dione |
| 2,4-dione | |
| 5-methyl-1-(3,2′-bipyrid-4-ylmethyl)-3-(4-trifluoro- | 5-methyl-1-(3-hydroxymethylpyrid-4-ylmethyl)-3-(4- |
| methanesulfanylphenyl)imidazolidine-2,4-dione | trifluoromethanesulfanylphenyl)imidazolidine-2,4- |
| | dione |
| 5-methyl-1-(3-chloropyrid-4-ylmethyl)-3-(4- | 5-methyl-1-(3-fluoropyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-(3-bromopyrid-4-ylmethyl)-3-(4- | 5-methyl-1-(3-aminopyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-(3-methylaminopyrid-4-ylmethyl)-3-(4- | 5-methyl-1-(3-ethylaminopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5-methyl-1-(3-isopropylaminopyrid-4-ylmethyl)-3- | 5-methyl-1-(3-phenylaminopyrid-4-ylmethyl)-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-(3-cyclohexylaminopyrid-4-ylmethyl)-3- | 5-methyl-1-(3-diethylaminopyrid-4-ylmethyl)-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-(3-N-cyclohexyl-N-methylaminopyrid- | 5-methyl-1-(3-piperidinopyrid-4-ylmethyl)-3-(4- |
| 4-ylmethyl)-3-(4-trifluoro- | trifluoromethanesulfanylphenyl)imidazolidine-2,4- |
| methanesulfanylphenyl)imidazolidine-2,4-dione | dione |
| 5-methyl-1-(3-morpholinopyrid-4-ylmethyl)-3-(4- | 5-methyl-1-[3-(4-methylpiperazino)pyrid-4-ylmethyl]- |
| trifluoromethanesulfanylphenyl)imidazolidine-2,4- | 3-(4-trifluoromethanesulfanylphenyl)imidazolidine-2,4- |
| dione | dione |
| 5-methyl-1-[3-(1H-pyrazol-1-yl)pyrid-4-ylmethyl]- | 5-methyl-1-[3-(2-benzoylhydrazino)pyrid-4-ylmethyl]- |
| 3-(4-trifluoromethanesulfanylphenyl)imidazolidine- | 3-(4-trifluoromethanesulfanylphenyl)imidazolidine-2,4- |
| 2,4-dione | dione |
| 5-methyl-1-(3-benzoylaminopyrid-4-ylmethyl)-3-(4- | 5-methyl-1-[3-(4-chlorobenzoylamino)pyrid-4- |
| trifluoromethanesulfanylphenyl)imidazolidine-2,4- | ylmethyl]-3-(4-trifluoro- |
| dione | methanesulfanylphenyl)imidazolidine-2,4-dione |
| 5-methyl-1-[3-(3-chlorobenzoylamino)pyrid-4- | 5-methyl-1-[3-(2-chlorobenzoylamino)pyrid-4- |
| ylmethyl]-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-[3-(4-fluorobenzoylamino)pyrid-4- | 5-methyl-1-[3-(4-methoxybenzoylamino)pyrid-4- |
| ylmethyl]-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(3-acetamidopyrid-4-ylmethyl)-3-(4- | 5-methyl-1-(3-phenylacetamidopyrid-4-ylmethyl)-3-(4- |
| trifluoromethanesulfanylphenyl)imidazolidine-2,4- | trifluoromethanesulfanylphenyl)imidazolidine-2,4- |
| dione | dione |
| 5-methyl-1-[3-(4-methylpiperazino)acetamidopyrid- | 5-methyl-1-(3-cyclohexanylcarboxamidopyrid-4- |
| 4-ylmethyl]-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-[3-(1-methyl-4- | 5-methyl-1-[3-(2-furanylcarbonyl)aminopyrid-4- |
| piperidinyl)carbonylaminopyrid-4-ylmethyl]-3-(4- | ylmethyl]-3-(4-trifluoro- |
| trifluoromethanesulfanylphenyl)imidazolidine-2,4- | methanesulfanylphenyl)imidazolidine-2,4-dione |
| dione | |
| 5-methyl-1-(3-ethoxycarbonylaminopyrid-4- | 5-methyl-1-(3-isopropoxycarbonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-(3-butoxycarbonylaminopyrid-4- | 5-methyl-1-[3-(2- |
| ylmethyl)-3-(4-trifluoro- | diethylaminoethoxycarbonylamino)pyrid-4-ylmethyl]- |
| methanesulfanylphenyl)imidazolidine-2,4-dione | 3-(4-trifluoromethanesulfanylphenyl)imidazolidine-2,4- |
| | dione |
| 5-methyl-1-(3-phenoxycarbonylaminopyrid-4- | 5-methyl-1-[3-(4-chloroanilino)carbonylaminopyrid-4- |
| ylmethyl)-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(3-anilinocarbonylaminopyrid-4- | 5-methyl-1-(3-benzylaminocarbonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-[3-(2- | 5-methyl-1-(3-ureidopyrid-4-ylmethyl)-3-(4-trifluoro- |
| pyridylamino)carbonylaminopyrid-4-ylmethyl]-3-(4- | methanesulfanylphenyl)imidazolidine-2,4-dione |
| trifluoromethanesulfanylphenyl)imidazolidine-2,4- | |
| dione | |
| 5-methyl-1-(3-benzylaminosulfonylaminopyrid-4- | 5-methyl-1-(3-methylaminosulfonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-(3-aminosulfonylaminopyrid-4- | 5-methyl-1-(3-morpholinosulfonylaminopyrid-4- |
| ylmethyl)-3-(4-trifluoro- | ylmethyl)-3-(4-trifluoro- |
| methanesulfanylphenyl)imidazolidine-2,4-dione | methanesulfanylphenyl)imidazolidine-2,4-dione |
| 5-methyl-1-(3-dimethylaminosulfonylaminopyrid-4- | 5-methyl-1-(3-anilinosulfonylaminopyrid-4-ylmethyl)- |
| ylmethyl)-3-(4-trifluoro- | 3-(4-trifluoromethanesulfanylphenyl)imidazolidine-2,4- |
| methanesulfanylphenyl)imidazolidine-2,4-dione | dione |
| 5-methyl-1-(3-phenylsulfonylaminopyrid-4- | 5-methyl-1-[3-(2-thienyl)sulfonylaminopyrid-4- |
| ylmethyl)-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(3-methylsulfonylaminopyrid-4- | 5-methyl-1-[3-(4-chlorophenyl)sulfonylaminopyrid-4- |
| ylmethyl)-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(3-benzylsulfonylaminopyrid-4- | 5-methyl-1-(3-hydroxycarbonylpyrid-4-ylmethyl)-3-(4- |
| ylmethyl)-3-(4-trifluoro- | trifluoromethanesulfanylphenyl)imidazolidine-2,4- |
| methanesulfanylphenyl)imidazolidine-2,4-dione | dione |
| 5-methyl-1-(3-ethoxycarbonylpyrid-4-ylmethyl)-3- | 5-methyl-1-(3-tert-butoxycarbonylpyrid-4-ylmethyl)-3- |
| (4-trifluoromethanesulfanylphenyl)imidazolidine- | (4-trifluoromethanesulfanylphenyl)imidazolidine-2,4- |
| 2,4-dione | dione |
| 5-methyl-1-(3-phenoxycarbonylpyrid-4-ylmethyl)-3- | 5-methyl-1-[3-(4-methylpiperazinocarbonyl)pyrid-4- |
| (4-trifluoromethanesulfanylphenyl)imidazolidine- | ylmethyl]-3-(4-trifluoro- |
| 2,4-dione | methanesulfanylphenyl)imidazolidine-2,4-dione |
| 5-methyl-1-[3-(methylamino)carbonylpyrid-4- | 5-methyl-1-[3-(dimethylamino)carbonylpyrid-4- |
| ylmethyl]-3-(4-trifluoro- | ylmethyl]-3-(4-trifluoro- |
| methanesulfanylphenyl)imidazolidine-2,4-dione | methanesulfanylphenyl)imidazolidine-2,4-dione |
| 5-methyl-1-(3-piperidinocarbonylpyrid-4-ylmethyl)- | 5-methyl-1-(3-morpholinocarbonylpyrid-4-ylmethyl)-3- |
| 3-(4-trifluoromethanesulfanylphenyl)imidazolidine- | (4-trifluoromethanesulfanylphenyl)imidazolidine-2,4- |
| 2,4-dione | dione |
| 5-methyl-1-[3-(4-phenylpiperazinocarbonyl)pyrid-4- | 5-methyl-1-(3-phenylaminocarbonylpyrid-4-ylmethyl)- |
| ylmethyl]-3-(4-trifluoro- | 3-(4-trifluoromethanesulfanylphenyl)imidazolidine-2,4- |
| methanesulfanylphenyl)imidazolidine-2,4-dione | dione |
| 5-methyl-1-[3-(methylphenylamino)carbonylpyrid- | 5-methyl-1-[3-(4- |
| 4-ylmethyl]-3-(4-trifluoro- | methoxyphenyl)methylaminocarbonylpyrid-4- |
| methanesulfanylphenyl)imidazolidine-2,4-dione | ylmethyl]-3-(4-trifluoro- |
| | methanesulfanylphenyl)imidazolidine-2,4-dione |
| 5-methyl-1-[3-(4- | 5-methyl-1-[3-(4-morpholinylamino)carbonylpyrid-4- |
| chlorophenyl)methylaminocarbonylpyrid-4- | ylmethyl]-3-(4-trifluoro- |
| ylmethyl]-3-(4-trifluoro- | methanesulfanylphenyl)imidazolidine-2,4-dione |
| methanesulfanylphenyl)imidazolidine-2,4-dione | |
| 5-methyl-1-(3-aminocarbonylpyrid-4-ylmethyl)-3- | 5-methyl-1-(3-phenoxypyrid-4-ylmethyl)-3-(4- |
| (4-trifluoromethanesulfanylphenyl)imidazolidine- | trifluoromethanesulfanylphenyl)imidazolidine-2,4- |
| 2,4-dione | dione |
| 5-methyl-1-(3-methoxypyrid-4-ylmethyl)-3-(4- | 5-methyl-1-(3-ethoxypyrid-4-ylmethyl)-3-(4-trifluoro- |
| trifluoromethanesulfanylphenyl)imidazolidine-2,4- | methanesulfanylphenyl)imidazolidine-2,4-dione |
| dione | |
| 5-methyl-1-[3-(1-pyrrolidinyl)ethoxypyrid-4- | 5-methyl-1-(3-benzyloxypyrid-4-ylmethyl)-3-(4- |
| ylmethyl]-3-(4-trifluoro- | trifluoromethanesulfanylphenyl)imidazolidine-2,4- |
| methanesulfanylphenyl)imidazolidine-2,4-dione | dione |
| 5-methyl-1-(3-benzoyloxypyrid-4-ylmethyl)-3-(4- | 5-methyl-1-[3-(4,5-dihydro-1H-imidazol-2-yl)pyrid-4- |
| trifluoromethanesulfanylphenyl)imidazolidine-2,4- | ylmethyl]-3-(4-trifluoro- |
| dione | methanesulfanylphenyl)imidazolidine-2,4-dione |
| 5-methyl-1-[3-(1H-imidazol-2-yl)pyrid-4-ylmethyl]- | 1-[2-(4-fluorophenyl)pyrid-4-ylmethyl]-3-(4-trifluoro- |
| 3-(4-trifluoromethanesulfanylphenyl)imidazolidine- | methanesulfonylphenyl)imidazolidine-2,4-dione |
| 2,4-dione | |
| 5-methyl-1-[3-(1H-tetrazol-5-yl)pyrid-4-ylmethyl]- | 1-[2-(4-methoxyphenyl)pyrid-4-ylmethyl]-3-(4- |
| 3-(4-trifluoromethanesulfanylphenyl)imidazolidine- | trifluoromethanesulfonylphenyl)imidazolidine-2,4- |
| 2,4-dione | dione |
| 5-methyl-1-(3-amidinopyrid-4-ylmethyl)-3-(4- | 1-(2-benzylpyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 5-methyl-1-(3-dimethylaminomethylpyrid-4- | 1-(2,2′-bipyrid-4-ylmethyl)-3-(4-trifluoro- |
| ylmethyl)-3-(4-trifluoro- | methanesulfonylphenyl)imidazolidine-2,4-dione |
| methanesulfanylphenyl)imidazolidine-2,4-dione | |
| 5-methyl-1-(3-aminomethylpyrid-4-ylmethyl)-3-(4- | 1-(2-hydroxymethylpyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-methylpyrid-4-ylmethyl)-3-(4-trifluoro- | 1-(2-chloropyrid-4-ylmethyl)-3-(4-trifluoro- |
| methanesulfanylphenyl)imidazolidine-2,4-dione | methanesulfonylphenyl)imidazolidine-2,4-dione |
| 1-(2-ethylpyrid-4-ylmethyl)-3-(4-trifluoro- | 1-(2-fluoropyrid-4-ylmethyl)-3-(4-trifluoro- |
| methanesulfanylphenyl)imidazolidine-2,4-dione | methanesulfonylphenyl)imidazolidine-2,4-dione |
| 1-(2-isopropylpyrid-4-ylmethyl)-3-(4-trifluoro- | 1-(2-bromopyrid-4-ylmethyl)-3-(4-trifluoro- |
| methanesulfanylphenyl)imidazolidine-2,4-dione | methanesulfonylphenyl)imidazolidine-2,4-dione |
| 1-(2-tert-butylpyrid-4-ylmethyl)-3-(4-trifluoro- | 1-(2-aminopyrid-4-ylmethyl)-3-(4-trifluoro- |
| methanesulfanylphenyl)imidazolidine-2,4-dione | methanesulfonylphenyl)imidazolidine-2,4-dione |
| 1-(2-isobutylpyrid-4-ylmethyl)-3-(4-trifluoro- | 1-(2-methylaminopyrid-4-ylmethyl)-3-(4-trifluoro- |
| methanesulfanylphenyl)imidazolidine-2,4-dione | methanesulfonylphenyl)imidazolidine-2,4-dione |
| 1-(2-cyclohexylpyrid-4-ylmethyl)-3-(4-trifluoro- | 1-(2-ethylaminopyrid-4-ylmethyl)-3-(4-trifluoro- |
| methanesulfanylphenyl)imidazolidine-2,4-dione | methanesulfonylphenyl)imidazolidine-2,4-dione |
| 1-(2-phenylpyrid-4-ylmethyl)-3-(4-trifluoro- | 1-(2-isopropylaminopyrid-4-ylmethyl)-3-(4-trifluoro- |
| methanesulfanylphenyl)imidazolidine-2,4-dione | methanesulfonylphenyl)imidazolidine-2,4-dione |
| 1-[2-(4-chlorophenyl)pyrid-4-ylmethyl]-3-(4- | 1-(2-phenylaminopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[2-(3-chlorophenyl)pyrid-4-ylmethyl]-3-(4- | 1-(2-cyclohexylaminopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[2-(2-chlorophenyl)pyrid-4-ylmethyl]-3-(4- | 1-(2-diethylaminopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[2-(4-fluorophenyl)pyrid-4-ylmethyl]-3-(4- | 1-(2-N-cyclohexyl-N-methylaminopyrid-4-ylmethyl)-3- |
| trifluoromethanesulfanylphenyl)imidazolidine-2,4- | (4-trifluoromethanesulfonylphenyl)imidazolidine-2,4- |
| dione | dione |
| 1-[2-(4-methoxyphenyl)pyrid-4-ylmethyl]-3-(4- | 1-(2-piperidinopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-benzylpyrid-4-ylmethyl)-3-(4-trifluoro- | 1-(2-morpholinopyrid-4-ylmethyl)-3-(4-trifluoro- |
| methanesulfanylphenyl)imidazolidine-2,4-dione | methanesulfonylphenyl)imidazolidine-2,4-dione |
| 1-(2,2′-bipyrid-4-ylmethyl)-3-(4-trifluoro- | 1-[2-(4-methylpiperazino)diethylaminopyrid-4- |
| methanesulfanylphenyl)imidazolidine-2,4-dione | ylmethyl]-3-(4-trifluoro- |
| | methanesulfonylphenyl)imidazolidine-2,4-dione |
| 1-(2-hydroxymethylpyrid-4-ylmethyl)-3-(4- | 1-[2-(1H-pyrazol-1-yl)pyrid-4-ylmethyl]-3-(4-trifluoro- |
| trifluoromethanesulfanylphenyl)imidazolidine-2,4- | methanesulfonylphenyl)imidazolidine-2,4-dione |
| dione | |
| 1-(2-chloropyrid-4-ylmethyl)-3-(4-trifluoro- | 1-[2-(2-benzoylhydrazino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(2-fluoropyrid-4-ylmethyl)-3-(4-trifluoro- | 1-(2-benzoylaminopyrid-4-ylmethyl)-3-(4-trifluoro- |
| methanesulfanylphenyl)imidazolidine-2,4-dione | methanesulfonylphenyl)imidazolidine-2,4-dione |
| 1-(2-bromopyrid-4-ylmethyl)-3-(4-trifluoro- | 1-[2-(4-chlorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(2-aminopyrid-4-ylmethyl)-3-(4-trifluoro- | 1-[2-(3-chlorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(2-methylaminopyrid-4-ylmethyl)-3-(4-trifluoro- | 1-[2-(2-chlorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(2-ethylaminopyrid-4-ylmethyl)-3-(4-trifluoro- | 1-[2-(4-fluorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(2-isopropylaminopyrid-4-ylmethyl)-3-(4- | 1-[2-(4-methoxybenzoylamino)pyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-(2-phenylaminopyrid-4-ylmethyl)-3-(4-trifluoro- | 1-(2-acetamidopyrid-4-ylmethyl)-3-(4-trifluoro- |
| methanesulfanylphenyl)imidazolidine-2,4-dione | methanesulfonylphenyl)imidazolidine-2,4-dione |
| 1-(2-cyclohexylaminopyrid-4-ylmethyl)-3-(4- | 1-(2-phenylacetamidopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-diethylaminopyrid-4-ylmethyl)-3-(4-trifluoro- | 1-[2-(4-methylpiperazino)acetamidopyrid-4-ylmethyl]- |
| methanesulfanylphenyl)imidazolidine-2,4-dione | 3-(4-trifluoromethanesulfonylphenyl)imidazolidine-2,4- |
| | dione |
| 1-(2-N-cyclohexyl-N-methylaminopyrid-4- | 1-(2-cyclohexanylcarboxamidopyrid-4-ylmethyl)-3-(4- |
| ylmethyl)-3-(4-trifluoro- | trifluoromethanesulfonylphenyl)imidazolidine-2,4- |
| methanesulfanylphenyl)imidazolidine-2,4-dione | dione |
| 1-(2-piperidinopyrid-4-ylmethyl)-3-(4-trifluoro- | 1-[2-(1-methyl-4-piperidinyl)carbonylaminopyrid-4- |
| methanesulfanylphenyl)imidazolidine-2,4-dione | ylmethyl]-3-(4-trifluoro- |
| | methanesulfonylphenyl)imidazolidine-2,4-dione |
| 1-(2-morpholinopyrid-4-ylmethyl)-3-(4-trifluoro- | 1-[2-(2-furanylcarbonyl)aminopyrid-4-ylmethyl]-3-(4- |
| methanesulfanylphenyl)imidazolidine-2,4-dione | trifluoromethanesulfonylphenyl)imidazolidine-2,4- |
| | dione |
| 1-[2-(4-methylpiperazino)pyrid-4-ylmethyl]-3-(4- | 1-(2-ethoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[2-(1H-pyrazol-1-yl)pyrid-4-ylmethyl]-3-(4- | 1-(2-isopropoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[2-(2-benzoylhydrazino)pyrid-4-ylmethyl]-3-(4- | 1-(2-butoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-(2-benzoylaminopyrid-4-ylmethyl)-3-(4-trifluoro- | 1-[2-(2-diethylaminoethoxycarbonylamino)pyrid-4- |
| methanesulfanylphenyl)imidazolidine-2,4-dione | ylmethyl]-3-(4-trifluoro- |
| | methanesulfonylphenyl)imidazolidine-2,4-dione |
| 1-[2-(4-chlorobenzoylamino)pyrid-4-ylmethyl]-3-(4- | 1-(2-phenoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[2-(3-chlorobenzoylamino)pyrid-4-ylmethyl]-3-(4- | 1-[2-(4-chloroanilino)carbonylaminopyrid-4- |
| trifluoromethanesulfanylphenyl)imidazolidine-2,4- | ylmethyl)]-3-(4-trifluoro- |
| dione | methanesulfonylphenyl)imidazolidine-2,4-dione |
| 1-[2-(2-chlorobenzoylamino)pyrid-4-ylmethyl]-3-(4- | 1-(2-anilinocarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[2-(4-fluorobenzoylamino)pyrid-4-ylmethyl]-3-(4- | 1-(2-benzylaminocarbonylaminopyrid-4-ylmethyl)-3- |
| trifluoromethanesulfanylphenyl)imidazolidine-2,4- | (4-trifluoromethanesulfonylphenyl)imidazolidine-2,4- |
| dione | dione |
| 1-[2-(4-methoxybenzoylamino)pyrid-4-ylmethyl]-3- | 1-[2-(2-pyridylamino)carbonylaminopyrid-4- |
| (4-trifluoromethanesulfanylphenyl)imidazolidine- | ylmethyl)]-3-(4-trifluoro- |
| 2,4-dione | methanesulfonylphenyl)imidazolidine-2,4-dione |
| 1-(2-acetamidopyrid-4-ylmethyl)-3-(4-trifluoro- | 1-(2-ureidopyrid-4-ylmethyl)-3-(4-trifluoro- |
| methanesulfanylphenyl)imidazolidine-2,4-dione | methanesulfonylphenyl)imidazolidine-2,4-dione |
| 1-(2-phenylacetamidopyrid-4-ylmethyl)-3-(4- | 1-(2-benzylaminosulfonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[2-(4-methylpiperazino)acetamidopyrid-4- | 1-(2-methylaminosulfonylaminopyrid-4-ylmethyl)-3- |
| ylmethyl]-3-(4-trifluoro- | (4-trifluoromethanesulfonylphenyl)imidazolidine-2,4- |
| methanesulfanylphenyl)imidazolidine-2,4-dione | dione |
| 1-(2-cyclohexanylcarboxamidopyrid-4-ylmethyl)-3- | 1-(2-aminosulfonylaminopyrid-4-ylmethyl)-3-(4- |
| (4-trifluoromethanesulfanylphenyl)imidazolidine- | trifluoromethanesulfonylphenyl)imidazolidine-2,4- |
| 2,4-dione | dione |
| 1-[2-(1-methyl-4-piperidinyl)carbonylaminopyrid-4- | 1-(2-morpholinosulfonylaminopyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-[2-(2-furanylcarbonyl)aminopyrid-4-ylmethyl]-3- | 1-(2-dimethylaminosulfonylaminopyrid-4-ylmethyl)-3- |
| (4-trifluoromethanesulfanylphenyl)imidazolidine- | (4-trifluoromethanesulfonylphenyl)imidazolidine-2,4- |
| 2,4-dione | dione |
| 1-(2-ethoxycarbonylaminopyrid-4-ylmethyl)-3-(4- | 1-(2-anilinosulfonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-(2-isopropoxycarbonylaminopyrid-4-ylmethyl)-3- | 1-(2-phenylsulfonylaminopyrid-4-ylmethyl)-3-(4- |
| (4-trifluoromethanesulfanylphenyl)imidazolidine- | trifluoromethanesulfonylphenyl)imidazolidine-2,4- |
| 2,4-dione | dione |
| 1-(2-butoxycarbonylaminopyrid-4-ylmethyl)-3-(4- | 1-[2-(2-thienyl)sulfonylaminopyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-[2-(2-diethylaminoethoxycarbonylamino)pyrid-4- | 1-(2-methylsulfonylaminopyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-(2-phenoxycarbonylaminopyrid-4-ylmethyl)-3-(4- | 1-[2-(4-chlorophenyl)sulfonylaminopyrid-4-ylmethyl]- |
| trifluoromethanesulfanylphenyl)imidazolidine-2,4- | 3-(4-trifluoromethanesulfonylphenyl)imidazolidine-2,4- |
| dione | dione |
| 1-[2-(4-chloroanilino)carbonylaminopyrid-4- | 1-(2-benzylsulfonylaminopyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-(2-anilinocarbonylaminopyrid-4-ylmethyl)-3-(4- | 1-(2-hydroxycarbonylpyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-benzylaminocarbonylaminopyrid-4-ylmethyl)- | 1-(2-ethoxycarbonylpyrid-4-ylmethyl)-3-(4-trifluoro- |
| 3-(4-trifluoromethanesulfanylphenyl)imidazolidine- | methanesulfonylphenyl)imidazolidine-2,4-dione |
| 2,4-dione | |
| 1-[2-(2-pyridylamino)carbonylaminopyrid-4- | 1-(2-tert-butoxycarbonylpyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-(2-ureidopyrid-4-ylmethyl)-3-(4-trifluoro- | 1-(2-phenoxycarbonylpyrid-4-ylmethyl)-3-(4-trifluoro- |
| methanesulfanylphenyl)imidazolidine-2,4-dione | methanesulfonylphenyl)imidazolidine-2,4-dione |
| 1-(2-benzylaminosulfonylaminopyrid-4-ylmethyl)- | 1-[2-(4-methylpiperazinocarbonyl)pyrid-4-ylmethyl]-3- |
| 3-(4-trifluoromethanesulfanylphenyl)imidazolidine- | (4-trifluoromethanesulfonylphenyl)imidazolidine-2,4- |
| 2,4-dione | dione |
| 1-(2-methylaminosulfonylaminopyrid-4-ylmethyl)- | 1-[2-(methylamino)carbonylpyrid-4-ylmethyl]-3-(4- |
| 3-(4-trifluoromethanesulfanylphenyl)imidazolidine- | trifluoromethanesulfonylphenyl)imidazolidine-2,4- |
| 2,4-dione | dione |
| 1-(2-aminosulfonylaminopyrid-4-ylmethyl)-3-(4- | 1-[2-(dimethylamino)carbonylpyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-(2-morpholinosulfonylaminopyrid-4-ylmethyl)-3- | 1-(2-piperidinocarbonylpyrid-4-ylmethyl)-3-(4- |
| (4-trifluoromethanesulfanylphenyl)imidazolidine- | trifluoromethanesulfonylphenyl)imidazolidine-2,4- |
| 2,4-dione | dione |
| 1-(2-dimethylaminosulfonylaminopyrid-4- | 1-(2-morpholinocarbonylpyrid-4-ylmethyl)-3-(4- |
| ylmethyl)-3-(4-trifluoro- | trifluoromethanesulfonylphenyl)imidazolidine-2,4- |
| methanesulfanylphenyl)imidazolidine-2,4-dione | dione |
| 1-(2-anilinosulfonylaminopyrid-4-ylmethyl)-3-(4- | 1-[2-(4-phenylpiperazinocarbonyl)pyrid-4-ylmethyl]-3- |
| trifluoromethanesulfanylphenyl)imidazolidine-2,4- | (4-trifluoromethanesulfonylphenyl)imidazolidine-2,4- |
| dione | dione |
| 1-(2-phenylsulfonylaminopyrid-4-ylmethyl)-3-(4- | 1-(2-phenylaminocarbonylpyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[2-(2-thienyl)sulfonylaminopyrid-4-ylmethyl]-3- | 1-[2-(methylphenylamino)carbonylpyrid-4-ylmethyl]-3- |
| (4-trifluoromethanesulfanylphenyl)imidazolidine- | (4-trifluoromethanesulfonylphenyl)imidazolidine-2,4- |
| 2,4-dione | dione |
| 1-(2-methylsulfonylaminopyrid-4-ylmethyl)-3-(4- | 1-[2-(4-methoxyphenyl)methylaminocarbonylpyrid-4- |
| trifluoromethanesulfanylphenyl)imidazolidine-2,4- | ylmethyl]-3-(4-trifluoro- |
| dione | methanesulfonylphenyl)imidazolidine-2,4-dione |
| 1-[2-(4-chlorophenyl)sulfonylaminopyrid-4- | 1-[2-(4-chlorophenyl)methylaminocarbonylpyrid-4- |
| ylmethyl]-3-(4-trifluoro- | ylmethyl]-3-(4-trifluoro- |
| methanesulfanylphenyl)imidazolidine-2,4-dione | methanesulfonylphenyl)imidazolidine-2,4-dione |
| 1-(2-benzylsulfonylaminopyrid-4-ylmethyl)-3-(4- | 1-[2-(4-morpholinylamino)carbonylpyrid-4-ylmethyl]- |
| trifluoromethanesulfanylphenyl)imidazolidine-2,4- | 3-(4-trifluoromethanesulfonylphenyl)imidazolidine-2,4- |
| dione | dione |
| 1-(2-hydroxycarbonylpyrid-4-ylmethyl)-3-(4- | 1-(2-aminocarbonylpyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-ethoxycarbonylpyrid-4-ylmethyl)-3-(4- | 1-(2-phenoxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-tert-butoxycarbonylpyrid-4-ylmethyl)-3-(4- | 1-(2-methoxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-phenoxycarbonylpyrid-4-ylmethyl)-3-(4- | 1-(2-ethoxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[2-(4-methylpiperazinocarbonyl)pyrid-4- | 1-[2-(1-pyrrolidinyl)ethoxypyrid-4-ylmethyl]-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-[2-(methylamino)carbonylpyrid-4-ylmethyl]-3-(4- | 1-(2-benzyloxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[2-(dimethylamino)carbonylpyrid-4-ylmethyl]-3- | 1-(2-benzoyloxypyrid-4-ylmethyl)-3-(4-trifluoro- |
| (4-trifluoromethanesulfanylphenyl)imidazolidine- | methanesulfonylphenyl)imidazolidine-2,4-dione |
| 2,4-dione | |
| 1-(2-piperidinocarbonylpyrid-4-ylmethyl)-3-(4- | 1-[2-(4,5-dihydro-1H-imidazol-2-yl)pyrid-4-ylmethyl]- |
| trifluoromethanesulfanylphenyl)imidazolidine-2,4- | 3-(4-trifluoromethanesulfonylphenyl)imidazolidine-2,4- |
| dione | dione |
| 1-(2-morpholinocarbonylpyrid-4-ylmethyl)-3-(4- | 1-[2-(1H-imidazol-2-yl)pyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-[2-(4-phenylpiperazinocarbonyl)pyrid-4- | 1-[2-(1H-tetrazol-5-yl)pyrid-4-ylmethyl]-3-(4-trifluoro- |
| ylmethyl]-3-(4-trifluoro- | methanesulfonylphenyl)imidazolidine-2,4-dione |
| methanesulfanylphenyl)imidazolidine-2,4-dione | |
| 1-(2-phenylaminocarbonylpyrid-4-ylmethyl)-3-(4- | 1-(2-amidinopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[2-(methylphenylamino)carbonylpyrid-4- | 1-(2-dimethylaminomethylpyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-[2-(4-methoxyphenyl)methylaminocarbonylpyrid- | 1-(2-aminomethylpyrid-4-ylmethyl)-3-(4-trifluoro- |
| 4-ylmethyl]-3-(4-trifluoro- | methanesulfonylphenyl)imidazolidine-2,4-dione |
| methanesulfanylphenyl)imidazolidine-2,4-dione | |
| 1-[2-(4-chlorophenyl)methylaminocarbonylpyrid-4- | 1-(3-methylpyrid-4-ylmethyl)-3-(4-trifluoro- |
| ylmethyl]-3-(4-trifluoro- | methanesulfonylphenyl)imidazolidine-2,4-dione |
| methanesulfanylphenyl)imidazolidine-2,4-dione | |
| 1-[2-(4-morpholinylamino)carbonylpyrid-4- | 1-(3-ethylpyrid-4-ylmethyl)-3-(4-trifluoro- |
| ylmethyl]-3-(4-trifluoro- | methanesulfbnylphenyl)imidazolidine-2,4-dione |
| methanesulfanylphenyl)imidazolidine-2,4-dione | |
| 1-(2-aminocarbonylpyrid-4-ylmethyl)-3-(4-trifluoro- | 1-(3-isopropylpyrid-4-ylmethyl)-3-(4-trifluoro- |
| methanesulfanylphenyl)imidazolidine-2,4-dione | methanesulfonylphenyl)imidazolidine-2,4-dione |
| 1-(2-phenoxypyrid-4-ylmethyl)-3-(4-trifluoro- | 1-(3-tert-butylpyrid-4-ylmethyl)-3-(4-trifluoro- |
| methanesulfanylphenyl)imidazolidine-2,4-dione | methanesulfonylphenyl)imidazolidine-2,4-dione |
| 1-(2-methoxypyrid-4-ylmethyl)-3-(4-trifluoro- | 1-(3-isobutylpyrid-4-ylmethyl)-3-(4-trifluoro- |
| methanesulfanylphenyl)imidazolidine-2,4-dione | methanesulfonylphenyl)imidazolidine-2,4-dione |
| 1-(2-ethoxypyrid-4-ylmethyl)-3-(4-trifluoro- | 1-(3-cyclohexylpyrid-4-ylmethyl)-3-(4-trifluoro- |
| methanesulfanylphenyl)imidazolidine-2,4-dione | methanesulfonylphenyl)imidazolidine-2,4-dione |
| 1-[2-(1-pyrrolidinyl)ethoxypyrid-4-ylmethyl]-3-(4- | 1-(3-phenylpyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-benzyloxypyrid-4-ylmethyl)-3-(4-trifluoro- | 1-[3-(4-chlorophenyl)pyrid-4-ylmethyl]-3-(4-trifluoro- |
| methanesulfanylphenyl)imidazolidine-2,4-dione | methanesulfonylphenyl)imidazolidine-2,4-dione |
| 1-(2-benzoyloxypyrid-4-ylmethyl)-3-(4-trifluoro- | 1-[3-(3-chlorophenyl)pyrid-4-ylmethyl]-3-(4-trifluoro- |
| methanesulfanylphenyl)imidazolidine-2,4-dione | methanesulfonylphenyl)imidazolidine-2,4-dione |
| 1-[2-(4,5-dihydro-1H-imidazol-2-yl)pyrid-4- | 1-[3-(2-chlorophenyl)pyrid-4-ylmethyl]-3-(4-trifluoro- |
| ylmethyl]-3-(4-trifluoro- | methanesulfonylphenyl)imidazolidine-2,4-dione |
| methanesulfanylphenyl)imidazolidine-2,4-dione | |
| 1-[2-(1H-imidazol-2-yl)pyrid-4-ylmethyl]-3-(4- | 1-[3-(4-fluorophenyl)pyrid-4-ylmethyl]-3-(4-trifluoro- |
| trifluoromethanesulfanylphenyl)imidazolidine-2,4- | methanesulfonylphenyl)imidazolidine-2,4-dione |
| dione | |
| 1-[2-(1H-tetrazol-5-yl)pyrid-4-ylmethyl]-3-(4- | 1-[3-(4-methoxyphenyl)pyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-(2-amidinopyrid-4-ylmethyl)-3-(4-trifluoro- | 1-(3-benzylpyrid-4-ylmethyl)-3-(4-trifluoro- |
| methanesulfanylphenyl)imidazolidine-2,4-dione | methanesulfonylphenyl)imidazolidine-2,4-dione |
| 1-(2-dimethylaminomethylpyrid-4-ylmethyl)-3-(4- | 1-(3,2′-bipyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(2-aminomethylpyrid-4-ylmethyl)-3-(4-trifluoro- | 1-(3-hydroxymethylpyrid-4-ylmethyl)-3-(4-trifluoro- |
| methanesulfanylphenyl)imidazolidine-2,4-dione | methanesulfonylphenyl)imidazolidine-2,4-dione |
| 1-(3-methylpyrid-4-ylmethyl)-3-(4-trifluoro- | 1-(3-chloropyrid-4-ylmethyl)-3-(4-trifluoro- |
| methanesulfanylphenyl)imidazolidine-2,4-dione | methanesulfonylphenyl)imidazolidine-2,4-dione |
| 1-(3-ethylpyrid-4-ylmethyl)-3-(4-trifluoro- | 1-(3-fluoropyrid-4-ylmethyl)-3-(4-trifluoro- |
| methanesulfanylphenyl)imidazolidine-2,4-dione | methanesulfonylphenyl)imidazolidine-2,4-dione |
| 1-(3-isopropylpyrid-4-ylmethyl)-3-(4-trifluoro- | 1-(3-bromopyrid-4-ylmethyl)-3-(4-trifluoro- |
| methanesulfanylphenyl)imidazolidine-2,4-dione | methanesulfonylphenyl)imidazolidine-2,4-dione |
| 1-(3-tert-butylpyrid-4-ylmethyl)-3-(4-trifluoro- | 1-(3-aminopyrid-4-ylmethyl)-3-(4-trifluoro- |
| methanesulfanylphenyl)imidazolidine-2,4-dione | methanesulfonylphenyl)imidazolidine-2,4-dione |
| 1-(3-isobutylpyrid-4-ylmethyl)-3-(4-trifluoro- | 1-(3-methylaminopyrid-4-ylmethyl)-3-(4-trifluoro- |
| methanesulfanylphenyl)imidazolidine-2,4-dione | methanesulfonylphenyl)imidazolidine-2,4-dione |
| 1-(3-cyclohexylpyrid-4-ylmethyl)-3-(4-trifluoro- | 1-(3-ethylaminopyrid-4-ylmethyl)-3-(4-trifluoro- |
| methanesulfanylphenyl)imidazolidine-2,4-dione | methanesulfonylphenyl)imidazolidine-2,4-dione |
| 1-(3-phenylpyrid-4-ylmethyl)-3-(4-trifluoro- | 1-(3-isopropylaminopyrid-4-ylmethyl)-3-(4-trifluoro- |
| methanesulfanylphenyl)imidazolidine-2,4-dione | methanesulfonylphenyl)imidazolidine-2,4-dione |
| 1-[3-(4-chlorophenyl)pyrid-4-ylmethyl]-3-(4- | 1-(3-phenylaminopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[3-(3-chlorophenyl)pyrid-4-ylmethyl]-3-(4- | 1-(3-cyclohexylaminopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[3-(2-chlorophenyl)pyrid-4-ylmethyl]-3-(4- | 1-(3-diethylaminopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[3-(4-fluorophenyl)pyrid-4-ylmethyl]-3-(4- | 1-(3-N-cyclohexyl-N-methylaminopyrid-4-ylmethyl)-3- |
| trifluoromethanesulfanylphenyl)imidazolidine-2,4- | (4-trifluoromethanesulfonylphenyl)imidazolidine-2,4- |
| dione | dione |
| 1-[3-(4-methoxyphenyl)pyrid-4-ylmethyl]-3-(4- | 1-(3-piperidinopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(3-benzylpyrid-4-ylmethyl)-3-(4-trifluoro- | 1-(3-morpholinopyrid-4-ylmethyl)-3-(4-trifluoro- |
| methanesulfanylphenyl)imidazolidine-2,4-dione | methanesulfonylphenyl)imidazolidine-2,4-dione |
| 1-(3,2′-bipyrid-4-ylmethyl)-3-(4-trifluoro- | 1-[3-(4-methylpiperazino)diethylaminopyrid-4- |
| methanesulfanylphenyl)imidazolidine-2,4-dione | ylmethyl]-3-(4-trifluoro- |
| | methanesulfonylphenyl)imidazolidine-2,4-dione |
| 1-(3-hydroxymethylpyrid-4-ylmethyl)-3-(4- | 1-[3-(1H-pyrazol-1-yl)pyrid-4-ylmethyl]-3-(4-trifluoro- |
| trifluoromethanesulfanylphenyl)imidazolidine-2,4- | methanesulfonylphenyl)imidazolidine-2,4-dione |
| dione | |
| 1-(3-chloropyrid-4-ylmethyl)-3-(4-trifluoro- | 1-[3-(2-benzoylhydrazino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(3-fluoropyrid-4-ylmethyl)-3-(4-trifluoro- | 1-(3-benzoylaminopyrid-4-ylmethyl)-3-(4-trifluoro- |
| methanesulfanylphenyl)imidazolidine-2,4-dione | methanesulfonylphenyl)imidazolidine-2,4-dione |
| 1-(3-bromopyrid-4-ylmethyl)-3-(4-trifluoro- | 1-[3-(4-chlorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(3-aminopyrid-4-ylmethyl)-3-(4-trifluoro- | 1-[3-(3-chlorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(3-methylaminopyrid-4-ylmethyl)-3-(4-trifluoro- | 1-[3-(2-chlorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(3-ethylaminopyrid-4-ylmethyl)-3-(4-trifluoro- | 1-[3-(4-fluorobenzoylamino)pyrid-4-ylmethyl]-3-(4- |
| methanesulfanylphenyl)imidazolidine-2,4-dione | trifluoromethanesulfonylphenyl)imidazolidine-2,4- |
| | dione |
| 1-(3-isopropylaminopyrid-4-ylmethyl)-3-(4- | 1-[3-(4-methoxybenzoylamino)pyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-(3-phenylaminopyrid-4-ylmethyl)-3-(4-trifluoro- | 1-(3-acetamidopyrid-4-ylmethyl)-3-(4-trifluoro- |
| methanesulfanylphenyl)imidazolidine-2,4-dione | methanesulfonylphenyl)imidazolidine-2,4-dione |
| 1-(3-cyclohexylaminopyrid-4-ylmethyl)-3-(4- | 1-(3-phenylacetamidopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(3-diethylaminopyrid-4-ylmethyl)-3-(4-trifluoro- | 1-[3-(4-methylpiperazino)acetamidopyrid-4-ylmethyl]- |
| methanesulfanylphenyl)imidazolidine-2,4-dione | 3-(4-trifluoromethanesulfonylphenyl)imidazolidine-2,4- |
| | dione |
| 1-(3-N-cyclohexyl-N-methylaminopyrid-4- | 1-(3-cyclohexanylcarboxamidopyrid-4-ylmethyl)-3-(4- |
| ylmethyl)-3-(4-trifluoro- | trifluoromethanesulfonylphenyl)imidazolidine-2,4- |
| methanesulfanylphenyl)imidazolidine-2,4-dione | dione |
| 1-(3-piperidinopyrid-4-ylmethyl)-3-(4-trifluoro- | 1-[3-(1-methyl-4-piperidinyl)carbonylaminopyrid-4- |
| methanesulfanylphenyl)imidazolidine-2,4-dione | ylmethyl]-3-(4-trifluoro- |
| | methanesulfonylphenyl)imidazolidine-2,4-dione |
| 1-(3-morpholinopyrid-4-ylmethyl)-3-(4-trifluoro- | 1-[3-(2-furanylcarbonyl)aminopyrid-4-ylmethyl]-3-(4- |
| methanesulfanylphenyl)imidazolidine-2,4-dione | trifluoromethanesulfonylphenyl)imidazolidine-2,4- |
| | dione |
| 1-[3-(4-methylpiperazino)pyrid-4-ylmethyl]-3-(4- | 1-(3-ethoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[3-(1H-pyrazol-1-yl)pyrid-4-ylmethyl]-3-(4- | 1-(3-isopropoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[3-(2-benzoylhydrazino)pyrid-4-ylmethyl]-3-(4- | 1-(3-butoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-(3-benzoylaminopyrid-4-ylmethyl)-3-(4-trifluoro- | 1-[3-(2-diethylaminoethoxycarbonylamino)pyrid-4- |
| methanesulfanylphenyl)imidazolidine-2,4-dione | ylmethyl]-3-(4-trifluoro- |
| | methanesulfonylphenyl)imidazolidine-2,4-dione |
| 1-[3-(4-chlorobenzoylamino)pyrid-4-ylmethyl]-3-(4- | 1-(3-phenoxycarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[3-(3-chlorobenzoylamino)pyrid-4-ylmethyl]-3-(4- | 1-[3-(4-chloroanilino)carbonylaminopyrid-4- |
| trifluoromethanesulfanylphenyl)imidazolidine-2,4- | ylmethyl)]-3-(4-trifluoro- |
| dione | methanesulfonylphenyl)imidazolidine-2,4-dione |
| 1-[3-(2-chlorobenzoylamino)pyrid-4-ylmethyl]-3-(4- | 1-(3-anilinocarbonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[3-(4-fluorobenzoylamino)pyrid-4-ylmethyl]-3-(4- | 1-(3-benzylaminocarbonylaminopyrid-4-ylmethyl)-3- |
| trifluoromethanesulfanylphenyl)imidazolidine-2,4- | (4-trifluoromethanesulfonylphenyl)imidazolidine-2,4- |
| dione | dione |
| 1-[3-(4-methoxybenzoylamino)pyrid-4-ylmethyl]-3- | 1-[3-(2-pyridylamino)carbonylaminopyrid-4- |
| (4-trifluoromethanesulfanylphenyl)imidazolidine- | ylmethyl)]-3-(4-trifluoro- |
| 2,4-dione | methanesulfonylphenyl)imidazolidine-2,4-dione |
| 1-(3-acetamidopyrid-4-ylmethyl)-3-(4-trifluoro- | 1-(3-ureidopyrid-4-ylmethyl)-3-(4-trifluoro- |
| methanesulfanylphenyl)imidazolidine-2,4-dione | methanesulfonylphenyl)imidazolidine-2,4-dione |
| 1-(3-phenylacetamidopyrid-4-ylmethyl)-3-(4- | 1-(3-benzylaminosulfonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[3-(4-methylpiperazino)acetamidopyrid-4- | 1-(3-methylaminosulfonylaminopyrid-4-ylmethyl)-3- |
| ylmethyl]-3-(4-trifluoro- | (4-trifluoromethanesulfonylphenyl)imidazolidine-2,4- |
| methanesulfanylphenyl)imidazolidine-2,4-dione | dione |
| 1-(3-cyclohexanylcarboxamidopyrid-4-ylmethyl)-3- | 1-(3-aminosulfonylaminopyrid-4-ylmethyl)-3-(4- |
| (4-trifluoromethanesulfanylphenyl)imidazolidine- | trifluoromethanesulfonylphenyl)imidazolidine-2,4- |
| 2,4-dione | dione |
| 1-[3-(1-methyl-4-piperidinyl)carbonylaminopyrid-4- | 1-(3-morpholinosulfonylaminopyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-[3-(2-furanylcarbonyl)aminopyrid-4-ylmethyl]-3- | 1-(3-dimethylaminosulfonylaminopyrid-4-ylmethyl)-3- |
| (4-trifluoromethanesulfanylphenyl)imidazolidine- | (4-trifluoromethanesulfonylphenyl)imidazolidine-2,4- |
| 2,4-dione | dione |
| 1-(3-ethoxycarbonylaminopyrid-4-ylmethyl)-3-(4- | 1-(3-anilinosulfonylaminopyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-(3-isopropoxycarbonylaminopyrid-4-ylmethyl)-3- | 1-(3-phenylsulfonylaminopyrid-4-ylmethyl)-3-(4- |
| (4-trifluoromethanesulfanylphenyl)imidazolidine- | trifluoromethanesulfonylphenyl)imidazolidine-2,4- |
| 2,4-dione | dione |
| 1-(3-butoxycarbonylaminopyrid-4-ylmethyl)-3-(4- | 1-[3-(2-thienyl)sulfonylaminopyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-[3-(2-diethylaminoethoxycarbonylamino)pyrid-4- | 1-(3-methylsulfonylaminopyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-(3-phenoxycarbonylaminopyrid-4-ylmethyl)-3-(4- | 1-[3-(4-chlorophenyl)sulfonylaminopyrid-4-ylmethyl]- |
| trifluoromethanesulfanylphenyl)imidazolidine-2,4- | 3-(4-trifluoromethanesulfonylphenyl)imidazolidine-2,4- |
| dione | dione |
| 1-[3-(4-chloroanilino)carbonylaminopyrid-4- | 1-(3-benzylsulfonylaminopyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-(3-anilinocarbonylaminopyrid-4-ylmethyl)-3-(4- | 1-(3-hydroxycarbonylpyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(3-benzylaminocarbonylaminopyrid-4-ylmethyl)- | 1-(3-ethoxycarbonylpyrid-4-ylmethyl)-3-(4-trifluoro- |
| 3-(4-trifluoromethanesulfanylphenyl)imidazolidine- | methanesulfonylphenyl)imidazolidine-2,4-dione |
| 2,4-dione | |
| 1-[3-(2-pyridylamino)carbonylaminopyrid-4- | 1-(3-tert-butoxycarbonylpyrid-4-ylmethyl)-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-(3-ureidopyrid-4-ylmethyl)-3-(4-trifluoro- | 1-(3-phenoxycarbonylpyrid-4-ylmethyl)-3-(4-trifluoro- |
| methanesulfanylphenyl)imidazolidine-2,4-dione | methanesulfonylphenyl)imidazolidine-2,4-dione |
| 1-(3-benzylaminosulfonylaminopyrid-4-ylmethyl)- | 1-[3-(4-methylpiperazinocarbonyl)pyrid-4-ylmethyl]-3- |
| 3-(4-trifluoromethanesulfanylphenyl)imidazolidine- | (4-trifluoromethanesulfonylphenyl)imidazolidine-2,4- |
| 2,4-dione | dione |
| 1-(3-methylaminosulfonylaminopyrid-4-ylmethyl)- | 1-[3-(methylamino)carbonylpyrid-4-ylmethyl]-3-(4- |
| 3-(4-trifluoromethanesulfanylphenyl)imidazolidine- | trifluoromethanesulfonylphenyl)imidazolidine-2,4- |
| 2,4-dione | dione |
| 1-(3-aminosulfonylaminopyrid-4-ylmethyl)-3-(4- | 1-[3-(dimethylamino)carbonylpyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-(3-morpholinosulfonylaminopyrid-4-ylmethyl)-3- | 1-(3-piperidinocarbonylpyrid-4-ylmethyl)-3-(4- |
| (4-trifluoromethanesulfanylphenyl)imidazolidine- | trifluoromethanesulfonylphenyl)imidazolidine-2,4- |
| 2,4-dione | dione |
| 1-(3-dimethylaminosulfonylaminopyrid-4- | 1-(3-morpholinocarbonylpyrid-4-ylmethyl)-3-(4- |
| ylmethyl)-3-(4-trifluoro- | trifluoromethanesulfonylphenyl)imidazolidine-2,4- |
| methanesulfanylphenyl)imidazolidine-2,4-dione | dione |
| 1-(3-anilinosulfonylaminopyrid-4-ylmethyl)-3-(4- | 1-[3-(4-phenylpiperazinocarbonyl)pyrid-4-ylmethyl]-3- |
| trifluoromethanesulfanylphenyl)imidazolidine-2,4- | (4-trifluoromethanesulfonylphenyl)imidazolidine-2,4- |
| dione | dione |
| 1-(3-phenylsulfonylaminopyrid-4-ylmethyl)-3-(4- | 1-(3-phenylaminocarbonylpyrid-4-ylmethyl)-3-(4- |
| trifluoromethanesulfanylphenyl)imidazolidine-2,4- | trifluoromethanesulfonylphenyl)imidazolidine-2,4- |
| dione | dione |
| 1-[3-(2-thienyl)sulfonylaminopyrid-4-ylmethyl]-3- | 1-[3-(methylphenylamino)carbonylpyrid-4-ylmethyl]-3- |
| (4-trifluoromethanesulfanylphenyl)imidazolidine- | (4-trifluoromethanesulfonylphenyl)imidazolidine-2,4- |
| 2,4-dione | dione |
| 1-(3-methylsulfonylaminopyrid-4-ylmethyl)-3-(4- | 1-[3-(4-methoxyphenyl)methylaminocarbonylpyrid-4- |
| trifluoromethanesulfanylphenyl)imidazolidine-2,4- | ylmethyl]-3-(4-trifluoro- |
| dione | methanesulfonylphenyl)imidazolidine-2,4-dione |
| 1-[3-(4-chlorophenyl)sulfonylaminopyrid-4- | 1-[3-(4-chlorophenyl)methylaminocarbonylpyrid-4- |
| ylmethyl]-3-(4-trifluoro- | ylmethyl]-3-(4-trifluoro- |
| methanesulfanylphenyl)imidazolidine-2,4-dione | methanesulfonylphenyl)imidazolidine-2,4-dione |
| 1-(3-benzylsulfonylaminopyrid-4-ylmethyl)-3-(4- | 1-[3-(4-morpholinylamino)carbonylpyrid-4-ylmethyl]- |
| trifluoromethanesulfanylphenyl)imidazolidine-2,4- | 3-(4-trifluoromethanesulfonylphenyl)imidazolidine-2,4- |
| dione | dione |
| 1-(3-hydroxycarbonylpyrid-4-ylmethyl)-3-(4- | 1-(3-aminocarbonylpyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(3-ethoxycarbonylpyrid-4-ylmethyl)-3-(4- | 1-(3-phenoxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(3-tert-butoxycarbonylpyrid-4-ylmethyl)-3-(4- | 1-(3-methoxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-(3-phenoxycarbonylpyrid-4-ylmethyl)-3-(4- | 1-(3-ethoxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[3-(4-methylpiperazinocarbonyl)pyrid-4- | 1-[3-(1-pyrrolidinyl)ethoxypyrid-4-ylmethyl]-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-[3-(methylamino)carbonylpyrid-4-ylmethyl]-3-(4- | 1-(3-benzyloxypyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[3-(dimethylamino)carbonylpyrid-4-ylmethyl]-3- | 1-(3-benzoyloxypyrid-4-ylmethyl)-3-(4-trifluoro- |
| (4-trifluoromethanesulfanylphenyl)imidazolidine- | methanesulfonylphenyl)imidazolidine-2,4-dione |
| 2,4-dione | |
| 1-(3-piperidinocarbonylpyrid-4-ylmethyl)-3-(4- | 1-[3-(4,5-dihydro-1H-imidazol-2-yl)pyrid-4-ylmethyl]- |
| trifluoromethanesulfanylphenyl)imidazolidine-2,4- | 3-(4-trifluoromethanesulfonylphenyl)imidazolidine-2,4- |
| dione | dione |
| 1-(3-morpholinocarbonylpyrid-4-ylmethyl)-3-(4- | 1-[3-(1H-imidazol-2-yl)pyrid-4-ylmethyl]-3-(4- |
| trifluoromethanesulfanylphenyl)imidazolidine-2,4- | trifluoromethanesulfonylphenyl)imidazolidine-2,4- |
| dione | dione |
| 1-[3-(4-phenylpiperazinocarbonyl)pyrid-4- | 1-[3-(1H-tetrazol-5-yl)pyrid-4-ylmethyl]-3-(4-trifluoro- |
| ylmethyl]-3-(4-trifluoro- | methanesulfonylphenyl)imidazolidine-2,4-dione |
| methanesulfanylphenyl)imidazolidine-2,4-dione | |
| 1-(3-phenylaminocarbonylpyrid-4-ylmethyl)-3-(4- | 1-(3-amidinopyrid-4-ylmethyl)-3-(4-trifluoro- |
| trifluoromethanesulfanylphenyl)imidazolidine-2,4- | methanesulfonylphenyl)imidazolidine-2,4-dione |
| dione | |
| 1-[3-(methylphenylamino)carbonylpyrid-4- | 1-(3-dimethylaminomethylpyridin)-4-ylmethyl]-3-(4- |
| ylmethyl]-3-(4-trifluoro- | trifluoromethanesulfonylphenyl)imidazolidine-2,4- |
| methanesulfanylphenyl)imidazolidine-2,4-dione | dione |
| 1-[3-(4-methoxyphenyl)methylaminocarbonylpyrid- | 1-(3-aminomethylpyrid-4-ylmethyl)-3-(4-trifluoro- |
| 4-ylmethyl]-3-(4-trifluoro- | methanesulfonylphenyl)imidazolidine-2,4-dione |
| methanesulfanylphenyl)imidazolidine-2,4-dione | |
| 1-[3-(4-chlorophenyl)methylaminocarbonylpyrid-4- | 5,5-dimethyl-1-(2-methylquinolin-4-ylmethyl)-3-(4- |
| ylmethyl]-3-(4-trifluoro- | trifluoromethoxyphenyl)imidazolidine-2,4-dione |
| methanesulfanylphenyl)imidazolidine-2,4-dione | |
| 1-[3-(4-morpholinylamino)carbonylpyrid-4- | 5,5-dimethyl-1-(2-methoxyquinolin-4-ylmethyl)-3-(4- |
| ylmethyl]-3-(4-trifluoro- | trifluoromethoxyphenyl)imidazolidine-2,4-dione |
| methanesulfanylphenyl)imidazolidine-2,4-dione | |
| 1-(3-aminocarbonylpyrid-4-ylmethyl)-3-(4-trifluoro- | 5,5-dimethyl-1-(2-chloroquinolin-4-ylmethyl)-3-(4- |
| methanesulfanylphenyl)imidazolidine-2,4-dione | trifluoromethoxyphenyl)imidazolidine-2,4-dione |
| 1-(3-phenoxypyrid-4-ylmethyl)-3-(4-trifluoro- | 5,5-dimethyl-1-(2-fluoroquinolin-4-ylmethyl)-3-(4- |
| methanesulfanylphenyl)imidazolidine-2,4-dione | trifluoromethoxyphenyl)imidazolidine-2,4-dione |
| 1-(3-methoxypyrid-4-ylmethyl)-3-(4-trifluoro- | 5,5-dimethyl-1-(2-aminoquinolin-4-ylmethyl)-3-(4- |
| methanesulfanylphenyl)imidazolidine-2,4-dione | trifluoromethoxyphenyl)imidazolidine-2,4-dione |
| 1-(3-ethoxypyrid-4-ylmethyl)-3-(4-trifluoro- | 5,5-dimethyl-1-(2-methylaminoquinolin-4-ylmethyl)-3- |
| methanesulfanylphenyl)imidazolidine-2,4-dione | (4-trifluoromethoxyphenyl)imidazolidine-2,4-dione |
| 1-[3-(1-pyrrolidinyl)ethoxypyrid-4-ylmethyl]-3-(4- | 5,5-dimethyl-1-(2-dimethylaminoquinolin-4-ylmethyl)- |
| trifluoromethanesulfanylphenyl)imidazolidine-2,4- | 3-(4-trifluoromethoxyphenyl)imidazolidine-2,4-dione |
| dione | |
| 1-(3-benzyloxypyrid-4-ylmethyl)-3-(4-trifluoro- | 5,5-dimethyl-1-(2-piperidinoquinolin-4-ylmethyl)-3-(4- |
| methanesulfanylphenyl)imidazolidine-2,4-dione | trifluoromethoxyphenyl)imidazolidine-2,4-dione |
| 1-(3-benzoyloxypyrid-4-ylmethyl)-3-(4-trifluoro- | 5,5-dimethyl-1-(3-methylquinolin-4-ylmethyl)-3-(4- |
| methanesulfanylphenyl)imidazolidine-2,4-dione | trifluoromethoxyphenyl)imidazolidine-2,4-dione |
| 1-[3-(4,5-dihydro-1H-imidazol-2-yl)pyrid-4- | 5,5-dimethyl-1-(3-methoxyquinolin-4-ylmethyl)-3-(4- |
| ylmethyl]-3-(4-trifluoro- | trifluoromethoxyphenyl)imidazolidine-2,4-dione |
| methanesulfanylphenyl)imidazolidine-2,4-dione | |
| 1-[3-(1H-imidazol-2-yl)pyrid-4-ylmethyl]-3-(4- | 5,5-dimethyl-1-(3-chloroquinolin-4-ylmethyl)-3-(4- |
| trifluoromethanesulfanylphenyl)imidazolidine-2,4- | trifluoromethoxyphenyl)imidazolidine-2,4-dione |
| dione | |
| 1-[3-(1H-tetrazol-5-yl)pyrid-4-ylmethyl]-3-(4- | 5,5-dimethyl-1-(3-fluoroquinolin-4-ylmethyl)-3-(4- |
| trifluoromethanesulfanylphenyl)imidazolidine-2,4- | trifluoromethoxyphenyl)imidazolidine-2,4-dione |
| dione | |
| 1-(3-amidinopyrid-4-ylmethyl)-3-(4-trifluoro- | 5,5-dimethyl-1-(3-aminoquinolin-4-ylmethyl)-3-(4- |
| methanesulfanylphenyl)imidazolidine-2,4-dione | trifluoromethoxyphenyl)imidazolidine-2,4-dione |
| 1-(3-dimethylaminomethylpyridin)-4-ylmethyl]-3- | 5,5-dimethyl-1-(3-methylaminoquinolin-4-ylmethyl)-3- |
| (4-trifluoromethanesulfanylphenyl)imidazolidine- | (4-trifluoromethoxyphenyl)imidazolidine-2,4-dione |
| 2,4-dione | |
| 1-(3-aminomethylpyrid-4-ylmethyl)-3-(4-trifluoro- | 5,5-dimethyl-1-(3-dimethylaminoquinolin-4-ylmethyl)- |
| methanesulfanylphenyl)imidazolidine-2,4-dione | 3-(4-trifluoromethoxyphenyl)imidazolidine-2,4-dione |
| 5,5-dimethyl-1-(2-methylpyrid-4-ylmethyl)-3-(4- | 5,5-dimethyl-1-(3-piperidinoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-ethylpyrid-4-ylmethyl)-3-(4- | 5,5-dimethyl-1-(5-methylquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-isopropylpyrid-4-ylmethyl)-3-(4- | 5,5-dimethyl-1-(5-methoxyquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-tert-butylpyrid-4-ylmethyl)-3-(4- | 5,5-dimethyl-1-(5-chloroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-isobutylpyrid-4-ylmethyl)-3-(4- | 5,5-dimethyl-1-(5-fluoroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-cyclohexylpyrid-4-ylmethyl)-3- | 5,5-dimethyl-1-(5-aminoquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(2-phenylpyrid-4-ylmethyl)-3-(4- | 5,5-dimethyl-1-(5-methylaminoquinolin-4-ylmethyl)-3- |
| trifluoromethanesulfonylphenyl)imidazolidine-2,4- | (4-trifluoromethoxyphenyl)imidazolidine-2,4-dione |
| dione | |
| 5,5-dimethyl-1-[2-(4-chlorophenyl)pyrid-4- | 5,5-dimethyl-1-(5-dimethylaminoquinolin-4-ylmethyl)- |
| ylmethyl]-3-(4-trifluoro- | 3-(4-trifluoromethoxyphenyl)imidazolidine-2,4-dione |
| methanesulfonylphenyl)imidazolidine-2,4-dione | |
| 5,5-dimethyl-1-[2-(3-chlorophenyl)pyrid-4- | 5,5-dimethyl-1-(5-piperidinoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[2-(2-chlorophenyl)pyrid-4- | 5,5-dimethyl-1-(6-methylquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[2-(4-fluorophenyl)pyrid-4- | 5,5-dimethyl-1-(6-methoxyquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[2-(4-methoxyphenyl)pyrid-4- | 5,5-dimethyl-1-(6-chloroquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-(2-benzylpyrid-4-ylmethyl)-3-(4- | 5,5-dimethyl-1-(6-fluoroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2,2′-bipyrid-4-ylmethyl)-3-(4- | 5,5-dimethyl-1-(6-aminoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-hydroxymethylpyrid-4-ylmethyl)- | 5,5-dimethyl-1-(6-methylaminoquinolin-4-ylmethyl)-3- |
| 3-(4-trifluoromethanesulfonylphenyl)imidazolidine- | (4-trifluoromethoxyphenyl)imidazolidine-2,4-dione |
| 2,4-dione | |
| 5,5-dimethyl-1-(2-chloropyrid-4-ylmethyl)-3-(4- | 5,5-dimethyl-1-(6-dimethylaminoquinolin-4-ylmethyl)- |
| trifluoromethanesulfonylphenyl)imidazolidine-2,4- | 3-(4-trifluoromethoxyphenyl)imidazolidine-2,4-dione |
| dione | |
| 5,5-dimethyl-1-(2-fluoropyrid-4-ylmethyl)-3-(4- | 5,5-dimethyl-1-(6-piperidinoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-bromopyrid-4-ylmethyl)-3-(4- | 5,5-dimethyl-1-(7-methylquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-aminopyrid-4-ylmethyl)-3-(4- | 5,5-dimethyl-1-(7-methoxyquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-methylaminopyrid-4-ylmethyl)-3- | 5,5-dimethyl-1-(7-chloroquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(2-ethylaminopyrid-4-ylmethyl)-3- | 5,5-dimethyl-1-(7-fluoroquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(2-isopropylaminopyrid-4- | 5,5-dimethyl-1-(7-aminoquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(2-phenylaminopyrid-4-ylmethyl)-3- | 5,5-dimethyl-1-(7-methylaminoquinolin-4-ylmethyl)-3- |
| (4-trifluoromethanesulfonylphenyl)imidazolidine- | (4-trifluoromethoxyphenyl)imidazolidine-2,4-dione |
| 2,4-dione | |
| 5,5-dimethyl-1-(2-cyclohexylaminopyrid-4- | 5,5-dimethyl-1-(7-dimethylaminoquinolin-4-ylmethyl)- |
| ylmethyl)-3-(4-trifluoro- | 3-(4-trifluoromethoxyphenyl)imidazolidine-2,4-dione |
| methanesulfonylphenyl)imidazolidine-2,4-dione | |
| 5,5-dimethyl-1-(2-diethylaminopyrid-4-ylmethyl)-3- | 5,5-dimethyl-1-(7-piperidinoquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(2-N-cyclohexyl-N- | 5,5-dimethyl-1-(8-methylquinolin-4-ylmethyl)-3-(4- |
| methylaminopyrid-4-ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(2-piperidinopyrid-4-ylmethyl)-3-(4- | 5,5-dimethyl-1-(8-methoxyquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-morpholinopyrid-4-ylmethyl)-3- | 5,5-dimethyl-1-(8-chloroquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-[2-(4- | 5,5-dimethyl-1-(8-fluoroquinolin-4-ylmethyl)-3-(4- |
| methylpiperazino)diethylaminopyrid-4-ylmethyl]-3- | trifluoromethoxyphenyl)imidazolidine-2,4-dione |
| (4-trifluoromethanesulfonylphenyl)imidazolidine- | |
| 2,4-dione | |
| 5,5-dimethyl-1-[2-(1H-pyrazol-1-yl)pyrid-4- | 5,5-dimethyl-1-(8-aminoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[2-(2-benzoylhydrazino)pyrid-4- | 5,5-dimethyl-1-(8-methylaminoquinolin-4-ylmethyl)-3- |
| ylmethyl]-3-(4-trifluoro- | (4-trifluoromethoxyphenyl)imidazolidine-2,4-dione |
| methanesulfonylphenyl)imidazolidine-2,4-dione | |
| 5,5-dimethyl-1-(2-benzoylaminopyrid-4-ylmethyl)- | 5,5-dimethyl-1-(8-dimethylaminoquinolin-4-ylmethyl)- |
| 3-(4-trifluoromethanesulfonylphenyl)imidazolidine- | 3-(4-trifluoromethoxyphenyl)imidazolidine-2,4-dione |
| 2,4-dione | |
| 5,5-dimethyl-1-[2-(4-chlorobenzoylamino)pyrid-4- | 5,5-dimethyl-1-(8-piperidinoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[2-(3-chlorobenzoylamino)pyrid-4- | 4-(2-methylquinolin)-4-ylmethyl-6-(4- |
| ylmethyl]-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(2-chlorobenzoylamino)pyrid-4- | 4-(2-methoxyquinolin)-4-ylmethyl-6-(4- |
| ylmethyl]-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(4-fluorobenzoylamino)pyrid-4- | 4-(2-chloroquinolin)-4-ylmethyl-6-(4- |
| ylmethyl]-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(4-methoxybenzoylamino)pyrid- | 4-(2-fluoroquinolin)-4-ylmethyl-6-(4- |
| 4-ylmethyl]-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-acetamidopyrid-4-ylmethyl)-3-(4- | 4-(2-aminoquinolin)-4-ylmethyl-6-(4- |
| trifluoromethanesulfonylphenyl)imidazolidine-2,4- | trifluoromethoxyphenyl)-4,6-diazaspiro[2.4]heptane- |
| dione | 5,7-dione |
| 5,5-dimethyl-1-(2-phenylacetamidopyrid-4- | 4-(2-methylaminoquinolin)-4-ylmethyl-6-(4-trifluoro- |
| ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-[2-(4- | 4-(2-dimethylaminoquinolin)-4-ylmethyl-6-(4-trifluoro- |
| methylpiperazino)acetamidopyrid-4-ylmethyl]-3-(4- | methoxyphenyl)-4,6-diazaspiro[2.4]heptane-5,7-dione |
| trifluoromethanesulfonylphenyl)imidazolidine-2,4- | |
| dione | |
| 5,5-dimethyl-1-(2-cyclohexanylcarboxamidopyrid- | 4-(2-piperidinoquinolin)-4-ylmethyl-6-(4-trifluoro- |
| 4-ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-[2-(1-methyl-4- | 4-(3-methylquinolin)-4-ylmethyl-6-(4- |
| piperidinyl)carbonylaminopyrid-4-ylmethyl]-3-(4- | trifluoromethoxyphenyl)-4,6-diazaspiro[2.4]heptane- |
| trifluoromethanesulfonylphenyl)imidazolidine-2,4- | 5,7-dione |
| dione | |
| 5,5-dimethyl-1-[2-(2-furanylcarbonyl)aminopyrid-4- | 4-(3-methoxyquinolin)-4-ylmethyl-6-(4- |
| ylmethyl]-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-ethoxycarbonylaminopyrid-4- | 4-(3-chloroquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-isopropoxycarbonylaminopyrid- | 4-(3-fluoroquinolin)-4-ylmethyl-6-(4- |
| 4-ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-butoxycarbonylaminopyrid-4- | 4-(3-aminoquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(2- | 4-(3-methylaminoquinolin)-4-ylmethyl-6-(4-trifluoro- |
| diethylaminoethoxycarbonylamino)pyrid-4- | methoxyphenyl)-4,6-diazaspiro[2.4]heptane-5,7-dione |
| ylmethyl]-3-(4-trifluoro- | |
| methanesulfonylphenyl)imidazolidine-2,4-dione | |
| 5,5-dimethyl-1-(2-phenoxycarbonylaminopyrid-4- | 4-(3-dimethylaminoquinolin)-4-ylmethyl-6-(4-trifluoro- |
| ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-[2-(4- | 4-(3-piperidinoquinolin)-4-ylmethyl-6-(4-trifluoro- |
| chloroanilino)carbonylaminopyrid-4-ylmethyl)]-3- | methoxyphenyl)-4,6-diazaspiro[2.4]heptane-5,7-dione |
| (4-trifluoromethanesulfonylphenyl)imidazolidine- | |
| 2,4-dione | |
| 5,5-dimethyl-1-(2-anilinocarbonylaminopyrid-4- | 4-(5-methylquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-benzylaminocarbonylaminopyrid- | 4-(5-methoxyquinolin)-4-ylmethyl-6-(4- |
| 4-ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(2- | 4-(5-chloroquinolin)-4-ylmethyl-6-(4- |
| pyridylamino)carbonylaminopyrid-4-ylmethyl)]-3- | trifluoromethoxyphenyl)-4,6-diazaspiro[2.4]heptane- |
| (4-trifluoromethanesulfonylphenyl)imidazolidine- | 5,7-dione |
| 2,4-dione | |
| 5,5-dimethyl-1-(2-ureidopyrid-4-ylmethyl)-3-(4- | 4-(5-fluoroquinolin)-4-ylmethyl-6-(4- |
| trifluoromethanesulfonylphenyl)imidazolidine-2,4- | trifluoromethoxyphenyl)-4,6-diazaspiro[2.4]heptane- |
| dione | 5,7-dione |
| 5,5-dimethyl-1-(2-benzylaminosulfonylaminopyrid- | 4-(5-aminoquinolin)-4-ylmethyl-6-(4- |
| 4-ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-methylaminosulfonylaminopyrid- | 4-(5-methylaminoquinolin)-4-ylmethyl-6-(4-trifluoro- |
| 4-ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-(2-aminosulfonylaminopyrid-4- | 4-(5-dimethylaminoquinolin)-4-ylmethyl-6-(4-trifluoro- |
| ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-(2-morpholinosulfonylaminopyrid-4- | 4-(5-piperidinoquinolin)-4-ylmethyl-6-(4-trifluoro- |
| ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-(2- | 4-(6-methylquinolin)-4-ylmethyl-6-(4- |
| dimethylaminosulfonylaminopyrid-4-ylmethyl)-3- | trifluoromethoxyphenyl)-4,6-diazaspiro[2.4]heptane- |
| (4-trifluoromethanesulfonylphenyl)imidazolidine- | 5,7-dione |
| 2,4-dione | |
| 5,5-dimethyl-1-(2-anilinosulfonylaminopyrid-4- | 4-(6-methoxyquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-phenylsulfonylaminopyrid-4- | 4-(6-chloroquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(2-thienyl)sulfonylaminopyrid-4- | 4-(6-fluoroquinolin)-4-ylmethyl-6-(4- |
| ylmethyl]-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-methylsulfonylaminopyrid-4- | 4-(6-aminoquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(4- | 4-(6-methylaminoquinolin)-4-ylmethyl-6-(4-trifluoro- |
| chlorophenyl)sulfonylaminopyrid-4-ylmethyl]-3-(4- | methoxyphenyl)-4,6-diazaspiro[2.4]heptane-5,7-dione |
| trifluoromethanesulfonylphenyl)imidazolidine-2,4- | |
| dione | |
| 5,5-dimethyl-1-(2-benzylsulfonylaminopyrid-4- | 4-(6-dimethylaminoquinolin)-4-ylmethyl-6-(4-trifluoro- |
| ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-(2-hydroxycarbonylpyrid-4- | 4-(6-piperidinoquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-ethoxycarbonylpyrid-4-ylmethyl)- | 4-(7-methylquinolin)-4-ylmethyl-6-(4- |
| 3-(4-trifluoromethanesulfonylphenyl)imidazolidine- | trifluoromethoxyphenyl)-4,6-diazaspiro[2.4]heptane- |
| 2,4-dione | 5,7-dione |
| 5,5-dimethyl-1-(2-tert-butoxycarbonylpyrid-4- | 4-(7-methoxyquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-(2-phenoxycarbonylpyrid-4- | 4-(7-chloroquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(4- | 4-(7-fluoroquinolin)-4-ylmethyl-6-(4- |
| methylpiperazinocarbonyl)pyrid-4-ylmethyl]-3-(4- | trifluoromethoxyphenyl)-4,6-diazaspiro[2.4]heptane- |
| trifluoromethanesulfonylphenyl)imidazolidine-2,4- | 5,7-dione |
| dione | |
| 5,5-dimethyl-1-[2-(methylamino)carbonylpyrid-4- | 4-(7-aminoquinolin)-4-ylmethyl-6-(4- |
| ylmethyl]-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2-(dimethylamino)carbonylpyrid-4- | 4-(7-methylaminoquinolin)-4-ylmethyl-6-(4-trifluoro- |
| ylmethyl]-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-(2-piperidinocarbonylpyrid-4- | 4-(7-dimethylaminoquinolin)-4-ylmethyl-6-(4-trifluoro- |
| ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-(2-morpholinocarbonylpyrid-4- | 4-(7-piperidinoquinolin)-4-ylmethyl-6-(4-trifluoro- |
| ylmethyl)-3-(4-trifluoro- | methoxyphenyl)-4,6-diazaspiro[2.4]heptane-5,7-dione |
| methanesulfonylphenyl)imidazolidine-2,4-dione | |
| 5,5-dimethyl-1-[2-(4- | 4-(8-methylquinolin)-4-ylmethyl-6-(4- |
| phenylpiperazinocarbonyl)pyrid-4-ylmethyl]-3-(4- | trifluoromethoxyphenyl)-4,6-diazaspiro[2.4]heptane- |
| trifluoromethanesulfonylphenyl)imidazolidine-2,4- | 5,7-dione |
| dione | |
| 5,5-dimethyl-1-(2-phenylaminocarbonylpyrid-4- | 4-(8-methoxyquinolin)-4-ylmethyl-6-(4- |
| ylmethyl)-3-(4-trifluoro- | trifluoromethoxyphenyl)-4,6-diazaspiro[2.4]heptane- |
| methanesulfonylphenyl)imidazolidine-2,4-dione | 5,7-dione |
| 5,5-dimethyl-1-[2- | 4-(8-chloroquinolin)-4-ylmethyl-6-(4- |
| (methylphenylamino)carbonylpyrid-4-ylmethyl]-3- | trifluoromethoxyphenyl)-4,6-diazaspiro[2.4]heptane- |
| (4-trifluoromethanesulfonylphenyl)imidazolidine- | 5,7-dione |
| 2,4-dione | |
| 5,5-dimethyl-1-[2-(4- | 4-(8-fluoroquinolin)-4-ylmethyl-6-(4- |
| methoxyphenyl)methylaminocarbonylpyrid-4- | trifluoromethoxyphenyl)-4,6-diazaspiro[2.4]heptane- |
| ylmethyl]-3-(4-trifluoro- | 5,7-dione |
| methanesulfonylphenyl)imidazolidine-2,4-dione | |
| 5,5-dimethyl-1-[2-(4- | 4-(8-aminoquinolin)-4-ylmethyl-6-(4- |
| chlorophenyl)methylaminocarbonylpyrid-4- | trifluoromethoxyphenyl)-4,6-diazaspiro[2.4]heptane- |
| ylmethyl]-3-(4-trifluoro- | 5,7-dione |
| methanesulfonylphenyl)imidazolidine-2,4-dione | |
| 5,5-dimethyl-1-[2-(4- | 4-(8-methylaminoquinolin)-4-ylmethyl-6-(4-trifluoro- |
| morpholinylamino)carbonylpyrid-4-ylmethyl]-3-(4- | methoxyphenyl)-4,6-diazaspiro[2.4]heptane-5,7-dione |
| trifluoromethanesulfonylphenyl)imidazolidine-2,4- | |
| dione | |
| 5,5-dimethyl-1-(2-aminocarbonylpyrid-4-ylmethyl)- | 4-(8-dimethylaminoquinolin)-4-ylmethyl-6-(4-trifluoro- |
| 3-(4-trifluoromethanesulfonylphenyl)imidazolidine- | methoxyphenyl)-4,6-diazaspiro[2.4]heptane-5,7-dione |
| 2,4-dione | |
| 5,5-dimethyl-1-(2-phenoxypyrid-4-ylmethyl)-3-(4- | 4-(8-piperidinoquinolin)-4-ylmethyl-6-(4-trifluoro- |
| trifluoromethanesulfonylphenyl)imidazolidine-2,4- | methoxyphenyl)-4,6-diazaspiro[2.4]heptane-5,7-dione |
| dione | |
| 5,5-dimethyl-1-(2-methoxypyrid-4-ylmethyl)-3-(4- | 5-methyl-1-(2-methylquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-ethoxypyrid-4-ylmethyl)-3-(4- | 5-methyl-1-(2-methoxyquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-[2-(1-pyrrolidinyl)ethoxypyrid-4- | 5-methyl-1-(2-chloroquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-(2-benzyloxypyrid-4-ylmethyl)-3-(4- | 5-methyl-1-(2-fluoroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-benzoyloxypyrid-4-ylmethyl)-3- | 5-methyl-1-(2-aminoquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-[2-(4,5-dihydro-1H-imidazol-2- | 5-methyl-1-(2-methylaminoquinolin-4-ylmethyl)-3-(4- |
| yl)pyrid-4-ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[2-(1H-imidazol-2-yl)pyrid-4- | 5-methyl-1-(2-dimethylaminoquinolin-4-ylmethyl)-3- |
| ylmethyl]-3-(4-trifluoro- | (4-trifluoromethoxyphenyl)imidazolidine-2,4-dione |
| methanesulfonylphenyl)imidazolidine-2,4-dione | |
| 5,5-dimethyl-1-[2-(1H-tetrazol-5-yl)pyrid-4- | 5-methyl-1-(2-piperidinoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-(2-amidinopyrid-4-ylmethyl)-3-(4- | 5-methyl-1-(3-methylquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(2-dimethylaminomethylpyrid-4- | 5-methyl-1-(3-methoxyquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(2-aminomethylpyrid-4-ylmethyl)-3- | 5-methyl-1-(3-chloroquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(3-methylpyrid-4-ylmethyl)-3-(4- | 5-methyl-1-(3-fluoroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-ethylpyrid-4-ylmethyl)-3-(4- | 5-methyl-1-(3-aminoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-isopropylpyrid-4-ylmethyl)-3-(4- | 5-methyl-1-(3-methylaminoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-tert-butylpyrid-4-ylmethyl)-3-(4- | 5-methyl-1-(3-dimethylaminoquinolin-4-ylmethyl)-3- |
| trifluoromethanesulfonylphenyl)imidazolidine-2,4- | (4-trifluoromethoxyphenyl)imidazolidine-2,4-dione |
| dione | |
| 5,5-dimethyl-1-(3-isobutylpyrid-4-ylmethyl)-3-(4- | 5-methyl-1-(3-piperidinoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-cyclohexylpyrid-4-ylmethyl)-3- | 5-methyl-1-(5-methylquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(3-phenylpyrid-4-ylmethyl)-3-(4- | 5-methyl-1-(5-methoxyquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-[3-(4-chlorophenyl)pyrid-4- | 5-methyl-1-(5-chloroquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(3-chlorophenyl)pyrid-4- | 5-methyl-1-(5-fluoroquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(2-chlorophenyl)pyrid-4- | 5-methyl-1-(5-aminoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(4-fluorophenyl)pyrid-4- | 5-methyl-1-(5-methylaminoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(4-methoxyphenyl)pyrid-4- | 5-methyl-1-(5-dimethylaminoquinolin-4-ylmethyl)-3- |
| ylmethyl]-3-(4-trifluoro- | (4-trifluoromethoxyphenyl)imidazolidine-2,4-dione |
| methanesulfonylphenyl)imidazolidine-2,4-dione | |
| 5,5-dimethyl-1-(3-benzylpyrid-4-ylmethyl)-3-(4- | 5-methyl-1-(5-piperidinoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3,2′-bipyrid-4-ylmethyl)-3-(4- | 5-methyl-1-(6-methylquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-hydroxymethylpyrid-4-ylmethyl)- | 5-methyl-1-(6-methoxyquinolin-4-ylmethyl)-3-(4- |
| 3-(4-trifluoromethanesulfonylphenyl)imidazolidine- | trifluoromethoxyphenyl)imidazolidine-2,4-dione |
| 2,4-dione | |
| 5,5-dimethyl-1-(3-chloropyrid-4-ylmethyl)-3-(4- | 5-methyl-1-(6-chloroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-fluoropyrid-4-ylmethyl)-3-(4- | 5-methyl-1-(6-fluoroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-bromopyrid-4-ylmethyl)-3-(4- | 5-methyl-1-(6-aminoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-aminopyrid-4-ylmethyl)-3-(4- | 5-methyl-1-(6-methylaminoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-methylaminopyrid-4-ylmethyl)-3- | 5-methyl-1-(6-dimethylaminoquinolin-4-ylmethyl)-3- |
| (4-trifluoromethanesulfonylphenyl)imidazolidine- | (4-trifluoromethoxyphenyl)imidazolidine-2,4-dione |
| 2,4-dione | |
| 5,5-dimethyl-1-(3-ethylaminopyrid-4-ylmethyl)-3- | 5-methyl-1-(6-piperidinoquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(3-isopropylaminopyrid-4- | 5-methyl-1-(7-methylquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-phenylaminopyrid-4-ylmethyl)-3- | 5-methyl-1-(7-methoxyquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(3-cyclohexylaminopyrid-4- | 5-methyl-1-(7-chloroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-diethylaminopyrid-4-ylmethyl)-3- | 5-methyl-1-(7-fluoroquinolin-4-ylmethyl)-3-(4- |
| (4-trifluoromethanesulfonylphenyl)imidazolidine- | trifluoromethoxyphenyl)imidazolidine-2,4-dione |
| 2,4-dione | |
| 5,5-dimethyl-1-(3-N-cyclohexyl-N- | 5-methyl-1-(7-aminoquinolin-4-ylmethyl)-3-(4- |
| methylaminopyrid-4-ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-piperidinopyrid-4-ylmethyl)-3-(4- | 5-methyl-1-(7-methylaminoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-morpholinopyrid-4-ylmethyl)-3- | 5-methyl-1-(7-dimethylaminoquinolin-4-ylmethyl)-3- |
| (4-trifluoromethanesulfonylphenyl)imidazolidine- | (4-trifluoromethoxyphenyl)imidazolidine-2,4-dione |
| 2,4-dione | |
| 5,5-dimethyl-1-[3-(4- | 5-methyl-1-(7-piperidinoquinolin-4-ylmethyl)-3-(4- |
| methylpiperazino)diethylaminopyrid-4-ylmethyl]-3- | trifluoromethoxyphenyl)imidazolidine-2,4-dione |
| (4-trifluoromethanesulfonylphenyl)imidazolidine- | |
| 2,4-dione | |
| 5,5-dimethyl-1-[3-(1H-pyrazol-1-yl)pyrid-4- | 5-methyl-1-(8-methylquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(2-benzoylhydrazino)pyrid-4- | 5-methyl-1-(8-methoxyquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-(3-benzoylaminopyrid-4-ylmethyl)- | 5-methyl-1-(8-chloroquinolin-4-ylmethyl)-3-(4- |
| 3-(4-trifluoromethanesulfonylphenyl)imidazolidine- | trifluoromethoxyphenyl)imidazolidine-2,4-dione |
| 2,4-dione | |
| 5,5-dimethyl-1-[3-(4-chlorobenzoylamino)pyrid-4- | 5-methyl-1-(8-fluoroquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(3-chlorobenzoylamino)pyrid-4- | 5-methyl-1-(8-aminoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(2-chlorobenzoylamino)pyrid-4- | 5-methyl-1-(8-methylaminoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(4-fluorobenzoylamino)pyrid-4- | 5-methyl-1-(8-dimethylaminoquinolin-4-ylmethyl)-3- |
| ylmethyl]-3-(4-trifluoro- | (4-trifluoromethoxyphenyl)imidazolidine-2,4-dione |
| methanesulfonylphenyl)imidazolidine-2,4-dione | |
| 5,5-dimethyl-1-[3-(4-methoxybenzoylamino)pyrid- | 5-methyl-1-(8-piperidinoquinolin-4-ylmethyl)-3-(4- |
| 4-ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-(3-acetamidopyrid-4-ylmethyl)-3-(4- | 1-(2-methylquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-phenylacetamidopyrid-4- | 1-(2-methoxyquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-[3-(4- | 1-(2-chloroquinolin-4-ylmethyl)-3-(4- |
| methylpiperazino)acetamidopyrid-4-ylmethyl]-3-(4- | trifluoromethoxyphenyl)imidazolidine-2,4-dione |
| trifluoromethanesulfonylphenyl)imidazolidine-2,4- | |
| dione | |
| 5,5-dimethyl-1-(3-cyclohexanylcarboxamidopyrid- | 1-(2-fluoroquinolin-4-ylmethyl)-3-(4- |
| 4-ylmethyl)-3-(4-trifluoro- | trifluoromethoxyphenyl)imidazolidine-2,4-dione |
| methanesulfonylphenyl)imidazolidine-2,4-dion | |
| 5,5-dimethyl-1-[3-(1-methyl-4- | 1-(2-aminoquinolin-4-ylmethyl)-3-(4- |
| piperidinyl)carbonylaminopyrid-4-ylmethyl]-3-(4- | trifluoromethoxyphenyl)imidazolidine-2,4-dione |
| trifluoromethanesulfonylphenyl)imidazolidine-2,4- | |
| dione | |
| 5,5-dimethyl-1-[3-(2-furanylcarbonyl)aminopyrid-4- | 1-(2-methylaminoquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-(3-ethoxycarbonylaminopyrid-4- | 1-(2-dimethylaminoquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-isopropoxycarbonylaminopyrid- | 1-(2-piperidinoquinolin-4-ylmethyl)-3-(4- |
| 4-ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-butoxycarbonylaminopyrid-4- | 1-(3-methylquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-[3-(2- | 1-(3-methoxyquinolin-4-ylmethyl)-3-(4- |
| diethylaminoethoxycarbonylamino)pyrid-4- | trifluoromethoxyphenyl)imidazolidine-2,4-dione |
| ylmethyl]-3-(4-trifluoro- | |
| methanesulfonylphenyl)imidazolidine-2,4-dione | |
| 5,5-dimethyl-1-(3-phenoxycarbonylaminopyrid-4- | 1-(3-chloroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-[3-(4- | 1-(3-fluoroquinolin-4-ylmethyl)-3-(4- |
| chloroanilino)carbonylaminopyrid-4-ylmethyl)]-3- | trifluoromethoxyphenyl)imidazolidine-2,4-dione |
| (4-trifluoromethanesulfonylphenyl)imidazolidine- | |
| 2,4-dione | |
| 5,5-dimethyl-1-(3-anilinocarbonylaminopyrid-4- | 1-(3-aminoquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-benzylaminocarbonylaminopyrid- | 1-(3-methylaminoquinolin-4-ylmethyl)-3-(4-trifluoro- |
| 4-ylmethyl)-3-(4-trifluoro- | methoxyphenyl)imidazolidine-2,4-dione |
| methanesulfonylphenyl)imidazolidine-2,4-dione | |
| 5,5-dimethyl-1-[3-(2- | 1-(3-dimethylaminoquinolin-4-ylmethyl)-3-(4-trifluoro- |
| pyridylamino)carbonylaminopyrid-4-ylmethyl)]-3- | methoxyphenyl)imidazolidine-2,4-dione |
| (4-trifluoromethanesulfonylphenyl)imidazolidine- | |
| 2,4-dione | |
| 5,5-dimethyl-1-(3-ureidopyrid-4-ylmethyl)-3-(4- | 1-(3-piperidinoquinolin-4-ylmethyl)-3-(4-trifluoro- |
| trifluoromethanesulfonylphenyl)imidazolidine-2,4- | methoxyphenyl)imidazolidine-2,4-dione |
| dione | |
| 5,5-dimethyl-1-(3-benzylaminosulfonylaminopyrid- | 1-(5-methylquinolin-4-ylmethyl)-3-(4- |
| 4-ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-methylaminosulfonylaminopyrid- | 1-(5-methoxyquinolin-4-ylmethyl)-3-(4- |
| 4-ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-aminosulfonylaminopyrid-4- | 1-(5-chloroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-morpholinosulfonylaminopyrid-4- | 1-(5-fluoroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3- | 1-(5-aminoquinolin-4-ylmethyl)-3-(4- |
| dimethylaminosulfonylaminopyrid-4-ylmethyl)-3- | trifluoromethoxyphenyl)imidazolidine-2,4-dione |
| (4-trifluoromethanesulfonylphenyl)imidazolidine- | |
| 2,4-dione | |
| 5,5-dimethyl-1-(3-anilinosulfonylaminopyrid-4- | 1-(5-methylaminoquinolin-4-ylmethyl)-3-(4-trifluoro- |
| ylmethyl)-3-(4-trifluoro- | methoxyphenyl)imidazolidine-2,4-dione |
| methanesulfonylphenyl)imidazolidine-2,4-dione | |
| 5,5-dimethyl-1-(3-phenylsulfonylaminopyrid-4- | 1-(5-dimethylaminoquinolin-4-ylmethyl)-3-(4-trifluoro- |
| ylmethyl)-3-(4-trifluoro- | methoxyphenyl)imidazolidine-2,4-dione |
| methanesulfonylphenyl)imidazolidine-2,4-dione | |
| 5,5-dimethyl-1-[3-(2-thienyl)sulfonylaminopyrid-4- | 1-(5-piperidinoquinolin-4-ylmethyl)-3-(4-trifluoro- |
| ylmethyl]-3-(4-trifluoro- | methoxyphenyl)imidazolidine-2,4-dione |
| methanesulfonylphenyl)imidazolidine-2,4-dione | |
| 5,5-dimethyl-1-(3-methylsulfonylaminopyrid-4- | 1-(6-methylquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-[3-(4- | 1-(6-methoxyquinolin-4-ylmethyl)-3-(4- |
| chlorophenyl)sulfonylaminopyrid-4-ylmethyl]-3-(4- | trifluoromethoxyphenyl)imidazolidine-2,4-dione |
| trifluoromethanesulfonylphenyl)imidazolidine-2,4- | |
| dione | |
| 5,5-dimethyl-1-(3-benzylsulfonylaminopyrid-4- | 1-(6-chloroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-hydroxycarbonylpyrid-4- | 1-(6-fluoroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-ethoxycarbonylpyrid-4-ylmethyl)- | 1-(6-aminoquinolin-4-ylmethyl)-3-(4- |
| 3-(4-trifluoromethanesulfonylphenyl)imidazolidine- | trifluoromethoxyphenyl)imidazolidine-2,4-dione |
| 2,4-dione | |
| 5,5-dimethyl-1-(3-tert-butoxycarbonylpyrid-4- | 1-(6-methylaminoquinolin-4-ylmethyl)-3-(4-trifluoro- |
| ylmethyl)-3-(4-trifluoro- | methoxyphenyl)imidazolidine-2,4-dione |
| methanesulfonylphenyl)imidazolidine-2,4-dione | |
| 5,5-dimethyl-1-(3-phenoxycarbonylpyrid-4- | 1-(6-dimethylaminoquinolin-4-ylmethyl)-3-(4-trifluoro- |
| ylmethyl)-3-(4-trifluoro- | methoxyphenyl)imidazolidine-2,4-dione |
| methanesulfonylphenyl)imidazolidine-2,4-dione | |
| 5,5-dimethyl-1-[3-(4- | 1-(6-piperidinoquinolin-4-ylmethyl)-3-(4-trifluoro- |
| methylpiperazinocarbonyl)pyrid-4-ylmethyl]-3-(4- | methoxyphenyl)imidazolidine-2,4-dione |
| trifluoromethanesulfonylphenyl)imidazolidine-2,4- | |
| dione | |
| 5,5-dimethyl-1-[3-(methylamino)carbonylpyrid-4- | 1-(7-methylquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-[3-(dimethylamino)carbonylpyrid-4- | 1-(7-methoxyquinolin-4-ylmethyl)-3-(4- |
| ylmethyl]-3-(4-trifluoro- | trifluoromethoxyphenyl)imidazolidine-2,4-dione |
| methanesulfonylphenyl)imidazolidine-2,4-dione | |
| 5,5-dimethyl-1-(3-piperidinocarbonylpyrid-4- | 1-(7-chloroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-(3-morpholinocarbonylpyrid-4- | 1-(7-fluoroquinolin-4-ylmethyl)-3-(4- |
| ylmethyl)-3-(4-trifluoro- | trifluoromethoxyphenyl)imidazolidine-2,4-dione |
| methanesulfonylphenyl)imidazolidine-2,4-dione | |
| 5,5-dimethyl-1-[3-(4- | 1-(7-aminoquinolin-4-ylmethyl)-3-(4- |
| phenylpiperazinocarbonyl)pyrid-4-ylmethyl]-3-(4- | trifluoromethoxyphenyl)imidazolidine-2,4-dione |
| trifluoromethanesulfonylphenyl)imidazolidine-2,4- | |
| dione | |
| 5,5-dimethyl-1-(3-phenylaminocarbonylpyrid-4- | 1-(7-methylaminoquinolin-4-ylmethyl)-3-(4-trifluoro- |
| ylmethyl)-3-(4-trifluoro- | methoxyphenyl)imidazolidine-2,4-dione |
| methanesulfonylphenyl)imidazolidine-2,4-dione | |
| 5,5-dimethyl-1-[3- | 1-(7-dimethylaminoquinolin-4-ylmethyl)-3-(4-trifluoro- |
| (methylphenylamino)carbonylpyrid-4-ylmethyl]-3- | methoxyphenyl)imidazolidine-2,4-dione |
| (4-trifluoromethanesulfonylphenyl)imidazolidine- | |
| 2,4-dione | |
| 5,5-dimethyl-1-[3-(4- | 1-(7-piperidinoquinolin-4-ylmethyl)-3-(4-trifluoro- |
| methoxyphenyl)methylaminocarbonylpyrid-4- | methoxyphenyl)imidazolidine-2,4-dione |
| ylmethyl]-3-(4-trifluoro- | |
| methanesulfonylphenyl)imidazolidine-2,4-dione | |
| 5,5-dimethyl-1-[3-(4- | 1-(8-methylquinolin-4-ylmethyl)-3-(4- |
| chlorophenyl)methylaminocarbonylpyrid-4- | trifluoromethoxyphenyl)imidazolidine-2,4-dione |
| ylmethyl]-3-(4-trifluoro- | |
| methanesulfonylphenyl)imidazolidine-2,4-dione | |
| 5,5-dimethyl-1-[3-(4- | 1-(8-methoxyquinolin-4-ylmethyl)-3-(4- |
| morpholinylamino)carbonylpyrid-4-ylmethyl]-3-(4- | trifluoromethoxyphenyl)imidazolidine-2,4-dione |
| trifluoromethanesulfonylphenyl)imidazolidine-2,4- | |
| dione | |
| 5,5-dimethyl-1-(3-aminocarbonylpyrid-4-ylmethyl)- | 1-(8-chloroquinolin-4-ylmethyl)-3-(4- |
| 3-(4-trifluoromethanesulfonylphenyl)imidazolidine- | trifluoromethoxyphenyl)imidazolidine-2,4-dione |
| 2,4-dione | |
| 5,5-dimethyl-1-(3-phenoxypyrid-4-ylmethyl)-3-(4- | 1-(8-fluoroquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-methoxypyrid-4-ylmethyl)-3-(4- | 1-(8-aminoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-ethoxypyrid-4-ylmethyl)-3-(4- | 1-(8-methylaminoquinolin-4-ylmethyl)-3-(4-trifluoro- |
| trifluoromethanesulfonylphenyl)imidazolidine-2,4- | methoxyphenyl)imidazolidine-2,4-dione |
| dione | |
| 5,5-dimethyl-1-[3-(1-pyrrolidinyl)ethoxypyrid-4- | 1-(8-dimethylaminoquinolin-4-ylmethyl)-3-(4-trifluoro- |
| ylmethyl]-3-(4-trifluoro- | methoxyphenyl)imidazolidine-2,4-dione |
| methanesulfonylphenyl)imidazolidine-2,4-dione | |
| 5,5-dimethyl-1-(3-benzyloxypyrid-4-ylmethyl)-3-(4- | 1-(8-piperidinoquinolin-4-ylmethyl)-3-(4- |
| trifluoromethanesulfonylphenyl)imidazolidine-2,4- | trifluoromethoxyphenyl)imidazolidine-2,4-dione |
| dione | |
| 5,5-dimethyl-1-(3-benzoyloxypyrid-4-ylmethyl)-3- | 5,5-dimethyl-1-(2-methylquinolin-4-ylmethyl)-3-(4- |
| (4-trifluoromethanesulfonylphenyl)imidazolidine- | trifluoromethanesulfanylphenyl)imidazolidine-2,4- |
| 2,4-dione | dione |
| 5,5-dimethyl-1-[3-(4,5-dihydro-1H-imidazol-2- | 5,5-dimethyl-1-(2-methoxyquinolin-4-ylmethyl)-3-(4- |
| yl)pyrid-4-ylmethyl]-3-(4-trifluoro- | trifluoromethanesulfanylphenyl)imidazolidine-2,4- |
| methanesulfonylphenyl)imidazolidine-2,4-dione | dione |
| 5,5-dimethyl-1-[3-(1H-imidazol-2-yl)pyrid-4- | 5,5-dimethyl-1-(2-chloroquinolin-4-ylmethyl)-3-(4- |
| ylmethyl]-3-(4-trifluoro- | trifluoromethanesulfanylphenyl)imidazolidine-2,4- |
| methanesulfonylphenyl)imidazolidine-2,4-dione | dione |
| 5,5-dimethyl-1-[3-(1H-tetrazol-5-yl)pyrid-4- | 5,5-dimethyl-1-(2-fluoroquinolin-4-ylmethyl)-3-(4- |
| ylmethyl]-3-(4-trifluoro- | trifluoromethanesulfanylphenyl)imidazolidine-2,4- |
| methanesulfonylphenyl)imidazolidine-2,4-dione | dione |
| 5,5-dimethyl-1-(3-amidinopyrid-4-ylmethyl)-3-(4- | 5,5-dimethyl-1-(2-aminoquinolin-4-ylmethyl)-3- |
| trifluoromethanesulfonylphenyl)imidazolidine-2,4- | (4-trifluoromethanesulfanylphenyl)imidazolidine-2,4- |
| dione | dione |
| 5,5-dimethyl-1-(3-dimethylaminomethylpyrid-4- | 5,5-dimethyl-1-(2-methylaminoquinolin-4-ylmethyl)-3- |
| ylmethyl)-3-(4-trifluoro- | (4-trifluoromethanesulfanylphenyl)imidazolidine-2,4- |
| methanesulfonylphenyl)imidazolidine-2,4-dione | dione |
| 5,5-dimethyl-1-(3-aminomethylpyrid-4-ylmethyl)-3- | 5,5-dimethyl-1-(2-dimethylaminoquinolin-4-ylmethyl)- |
| (4-trifluoromethanesulfonylphenyl)imidazolidine- | 3-(4-trifluoromethanesulfanylphenyl)imidazolidine-2,4- |
| 2,4-dione | dione |
| 4-(2-methylpyridin)-4-ylmethyl-6-(4-trifluoro- | 5,5-dimethyl-1-(2-piperidinoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-ethylpyridin)-4-ylmethyl-6-(4-trifluoro- | 5,5-dimethyl-1-(3-methylquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-isopropylpyridin)-4-ylmethyl-6-(4-trifluoro- | 5,5-dimethyl-1-(3-methoxyquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-tert-butylpyridin)-4-ylmethyl-6-(4-trifluoro- | 5,5-dimethyl-1-(3-chloroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-isobutylpyridin)-4-ylmethyl-6-(4-trifluoro- | 5,5-dimethyl-1-(3-fluoroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-cyclohexylpyridin)-4-ylmethyl-6-(4-trifluoro- | 5,5-dimethyl-1-(3-aminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-phenylpyridin)-4-ylmethyl-6-(4-trifluoro- | 5,5-dimethyl-1-(3-methylaminoquinolin-4-ylmethyl)-3- |
| methanesulfonylphenyl)-4,6-diazaspiro[2.4]heptane- | (4-trifluoromethanesulfanylphenyl)imidazolidine-2,4- |
| 5,7-dione | dione |
| 4-[2-(4-chlorophenyl)pyridin]-4-ylmethyl-6-(4- | 5,5-dimethyl-1-(3-dimethylaminoquinolin-4-ylmethyl)- |
| trifluoromethanesulfonylphenyl)-4,6- | 3-(4-trifluoromethanesulfanylphenyl)imidazolidine-2,4- |
| diazaspiro[2.4]heptane-5,7-dione | dione |
| 4-[2-(3-chlorophenyl)pyridin]-4-ylmethyl-6-(4- | 5,5-dimethyl-1-(3-piperidinoquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(2-chlorophenyl)pyridin]-4-ylmethyl-6-(4- | 5,5-dimethyl-1-(5-methylquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(4-fluorophenyl)pyridin]-4-ylmethyl-6-(4- | 5,5-dimethyl-1-(5-methoxyquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(4-methoxyphenyl)pyridin]-4-ylmethyl-6-(4- | 5,5-dimethyl-1-(5-chloroquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-(2-benzylpyridin)-4-ylmethyl-6-(4-trifluoro- | 5,5-dimethyl-1-(5-fluoroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2,2′-bipyrid-4-ylmethyl-6-(4-trifluoro- | 5,5-dimethyl-1-(5-aminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-hydroxymethylpyridin)-4-ylmethyl-6-(4- | 5,5-dimethyl-1-(5-methylaminoquinolin-4-ylmethyl)-3- |
| trifluoromethanesulfonylphenyl)-4,6- | (4-trifluoromethanesulfanylphenyl)imidazolidine-2,4- |
| diazaspiro[2.4]heptane-5,7-dione | dione |
| 4-(2-chloropyrid-4-ylmethyl-6-(4-trifluoro- | 5,5-dimethyl-1-(5-dimethylaminoquinolin-4-ylmethyl)- |
| methanesulfonylphenyl)-4,6-diazaspiro[2.4]heptane- | 3-(4-trifluoromethanesulfanylphenyl)imidazolidine-2,4- |
| 5,7-dione | dione |
| 4-(2-fluoropyrid-4-ylmethyl-6-(4-trifluoro- | 5,5-dimethyl-1-(5-piperidinoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-bromopyrid-4-ylmethyl-6-(4-trifluoro- | 5,5-dimethyl-1-(6-methylquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-aminopyrid-4-ylmethyl-6-(4-trifluoro- | 5,5-dimethyl-1-(6-methoxyquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-methylaminopyrid-4-ylmethyl-6-(4-trifluoro- | 5,5-dimethyl-1-(6-chloroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-ethylaminopyrid-4-ylmethyl-6-(4-trifluoro- | 5,5-dimethyl-1-(6-fluoroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-isopropylaminopyrid-4-ylmethyl-6-(4-trifluoro- | 5,5-dimethyl-1-(6-aminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-phenylaminopyrid-4-ylmethyl-6-(4-trifluoro- | 5,5-dimethyl-1-(6-methylaminoquinolin-4-ylmethyl)-3- |
| methanesulfonylphenyl)-4,6-diazaspiro[2.4]heptane- | (4-trifluoromethanesulfanylphenyl)imidazolidine-2,4- |
| 5,7-dione | dione |
| 4-(2-cyclohexylaminopyrid-4-ylmethyl-6-(4- | 5,5-dimethyl-1-(6-dimethylaminoquinolin-4-ylmethyl)- |
| trifluoromethanesulfonylphenyl)-4,6- | 3-(4-trifluoromethanesulfanylphenyl)imidazolidine-2,4- |
| diazaspiro[2.4]heptane-5,7-dione | dione |
| 4-(2-diethylaminopyrid-4-ylmethyl-6-(4-trifluoro- | 5,5-dimethyl-1-(6-piperidinoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-N-cyclohexyl-N-methylaminopyrid-4- | 5,5-dimethyl-1-(7-methylquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-(2-piperidinopyrid-4-ylmethyl-6-(4-trifluoro- | 5,5-dimethyl-1-(7-methoxyquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-morpholinopyrid-4-ylmethyl-6-(4-trifluoro- | 5,5-dimethyl-1-(7-chloroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-[2-(4-methylpiperazino)diethylaminopyrid-4- | 5,5-dimethyl-1-(7-fluoroquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-[2-(1H-pyrazol-1-yl)pyridin]-4-ylmethyl-6-(4- | 5,5-dimethyl-1-(7-aminoquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(2-benzoylhydrazino)pyrid-4-ylmethyl-6-(4- | 5,5-dimethyl-1-(7-methylaminoquinolin-4-ylmethyl)-3- |
| trifluoromethanesulfonylphenyl)-4,6- | (4-trifluoromethanesulfanylphenyl)imidazolidine-2,4- |
| diazaspiro[2.4]heptane-5,7-dione | dione |
| 4-(2-benzoylaminopyrid-4-ylmethyl-6-(4-trifluoro- | 5,5-dimethyl-1-(7-dimethylaminoquinolin-4-ylmethyl)- |
| methanesulfonylphenyl)-4,6-diazaspiro[2.4]heptane- | 3-(4-trifluoromethanesulfanylphenyl)imidazolidine-2,4- |
| 5,7-dione | dione |
| 4-[2-(4-chlorobenzoylamino)pyrid-4-ylmethyl-6-(4- | 5,5-dimethyl-1-(7-piperidinoquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(3-chlorobenzoylamino)pyrid-4-ylmethyl-6-(4- | 5,5-dimethyl-1-(8-methylquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(2-chlorobenzoylamino)pyrid-4-ylmethyl-6-(4- | 5,5-dimethyl-1-(8-methoxyquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(4-fluorobenzoylamino)pyrid-4-ylmethyl-6-(4- | 5,5-dimethyl-1-(8-chloroquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[2-(4-methoxybenzoylamino)pyrid-4-ylmethyl-6- | 5,5-dimethyl-1-(8-fluoroquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-(2-acetamidopyrid-4-ylmethyl-6-(4-trifluoro- | 5,5-dimethyl-1-(8-aminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-phenylacetamidopyrid-4-ylmethyl-6-(4- | 5,5-dimethyl-1-(8-methylaminoquinolin-4-ylmethyl)-3- |
| trifluoromethanesulfonylphenyl)-4,6- | (4-trifluoromethanesulfanylphenyl)imidazolidine-2,4- |
| diazaspiro[2.4]heptane-5,7-dione | dione |
| 4-[2-(4-methylpiperazino)acetamidopyrid-4- | 5,5-dimethyl-1-(8-dimethylaminoquinolin-4-ylmethyl)- |
| ylmethyl-6-(4-trifluoromethanesulfonylphenyl)-4,6- | 3-(4-trifluoromethanesulfanylphenyl)imidazolidine-2,4- |
| diazaspiro[2.4]heptane-5,7-dione | dione |
| 4-(2-cyclohexanylcarboxamidopyrid-4-ylmethyl-6- | 5,5-dimethyl-1-(8-piperidinoquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-[2-(1-methyl-4-piperidinyl)carbonylaminopyrid-4- | 4-(2-methylquinolin)-4-ylmethyl-6-(4-trifluoro- |
| ylmethyl-6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(2-furanylcarbonyl)aminopyrid-4-ylmethyl-6- | 4-(2-methoxyquinolin)-4-ylmethyl-6-(4-trifluoro- |
| (4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-ethoxycarbonylaminopyrid-4-ylmethyl-6-(4- | 4-(2-chloroquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-isopropoxycarbonylaminopyrid-4-ylmethyl-6- | 4-(2-fluoroquinolin)-4-ylmethyl-6-(4-trifluoro- |
| (4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-butoxycarbonylaminopyrid-4-ylmethyl-6-(4- | 4-(2-aminoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(2-diethylaminoethoxycarbonylamino)pyrid-4- | 4-(2-methylaminoquinolin)-4-ylmethyl-6-(4-trifluoro- |
| ylmethyl-6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-phenoxycarbonylaminopyrid-4-ylmethyl-6-(4- | 4-(2-dimethylaminoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(4-chloroanilino)carbonylaminopyrid-4- | 4-(2-piperidinoquinolin)-4-ylmethyl-6-(4-trifluoro- |
| ylmethyl)-6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-anilinocarbonylaminopyrid-4-ylmethyl-6-(4- | 4-(3-methylquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-benzylaminocarbonylaminopyrid-4-ylmethyl-6- | 4-(3-methoxyquinolin)-4-ylmethyl-6-(4-trifluoro- |
| (4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(2-pyridylamino)carbonylaminopyrid-4- | 4-(3-chloroquinolin)-4-ylmethyl-6-(4-trifluoromethane- |
| ylmethyl)-6-(4-trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-(2-ureidopyrid-4-ylmethyl-6-(4-trifluoro- | 4-(3-fluoroquinolin)-4-ylmethyl-6-(4-trifluoromethane- |
| methanesulfonylphenyl)-4,6-diazaspiro[2.4]heptane- | sulfanylphenyl)-4,6-diazaspiro[2.4]heptane-5,7-dione |
| 5,7-dione | |
| 4-(2-benzylaminosulfonylaminopyrid-4-ylmethyl-6- | 4-(3-aminoquinolin)-4-ylmethyl-6-(4-trifluoromethane- |
| (4-trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-(2-methylaminosulfonylaminopyrid-4-ylmethyl-6- | 4-(3-methylaminoquinolin)-4-ylmethyl-6-(4-trifluoro- |
| (4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-aminosulfonylaminopyrid-4-ylmethyl-6-(4- | 4-(3-dimethylaminoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-morpholinosulfonylaminopyrid-4-ylmethyl-6- | 4-(3-piperidinoquinolin)-4-ylmethyl-6-(4-trifluoro- |
| (4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-dimethylaminosulfonylaminopyrid-4-ylmethyl- | 4-(5-methylquinolin)-4-ylmethyl-6-(4- |
| 6-(4-trifluoromethanesulfonylphenyl)-4,6- | trifluoromethanesulfanylphenyl)-4,6- |
| diazaspiro[2.4]heptane-5,7-dione | diazaspiro[2.4]heptane-5,7-dione |
| 4-(2-anilinosulfonylaminopyrid-4-ylmethyl-6-(4- | 4-(5-methoxyquinolin)-4-ylmethyl-6-(4- |
| trifluoromethanesulfonylphenyl)-4,6- | trifluoromethanesulfanylphenyl)-4,6- |
| diazaspiro[2.4]heptane-5,7-dione | diazaspiro[2.4]heptane-5,7-dione |
| 4-(2-phenylsulfonylaminopyrid-4-ylmethyl-6-(4- | 4-(5-chloroquinolin)-4-ylmethyl-6-(4-trifluoromethane- |
| trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-[2-(2-thienyl)sulfonylaminopyrid-4-ylmethyl-6-(4- | 4-(5-fluoroquinolin)-4-ylmethyl-6-(4-trifluoromethane- |
| trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-(2-methylsulfonylaminopyrid-4-ylmethyl-6-(4- | 4-(5-aminoquinolin)-4-ylmethyl-6-(4-trifluoromethane- |
| trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-[2-(4-chlorophenyl)sulfonylaminopyrid-4- | 4-(5-methylaminoquinolin)-4-ylmethyl-6-(4-trifluoro- |
| ylmethyl-6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-benzylsulfonylaminopyrid-4-ylmethyl-6-(4- | 4-(5-dimethylaminoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-hydroxycarbonylpyridin)-4-ylmethyl-6-(4- | 4-(5-piperidinoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-ethoxycarbonylpyridin)-4-ylmethyl-6-(4- | 4-(6-methylquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-tert-butoxycarbonylpyridin)-4-ylmethyl-6-(4- | 4-(6-methoxyquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-phenoxycarbonylpyridin)-4-ylmethyl-6-(4- | 4-(6-chloroquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(4-methylpiperazinocarbonyl)pyridin]-4- | 4-(6-fluoroquinolin)-4-ylmethyl-6-(4-trifluoro- |
| ylmethyl-6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(methylamino)carbonylpyridin)-4-ylmethyl-6- | 4-(6-aminoquinolin)-4-ylmethyl-6-(4-trifluoromethane- |
| (4-trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-[2-(dimethylamino)carbonylpyridin)-4-ylmethyl- | 4-(6-methylaminoquinolin)-4-ylmethyl-6-(4-trifluoro- |
| 6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-piperidinocarbonylpyridin)-4-ylmethyl-6-(4- | 4-(6-dimethylaminoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-morpholinocarbonylpyridin)-4-ylmethyl-6-(4- | 4-(6-piperidinoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(4-phenylpiperazinocarbonyl)pyridin]-4- | 4-(7-methylquinolin)-4-ylmethyl-6-(4- |
| ylmethyl-6-(4-trifluoromethanesulfonylphenyl)-4,6- | trifluoromethanesulfanylphenyl)-4,6- |
| diazaspiro[2.4]heptane-5,7-dione | diazaspiro[2.4]heptane-5,7-dione |
| 4-(2-phenylaminocarbonylpyridin)-4-ylmethyl-6-(4- | 4-(7-methoxyquinolin)-4-ylmethyl-6-(4- |
| trifluoromethanesulfonylphenyl)-4,6- | trifluoromethanesulfanylphenyl)-4,6- |
| diazaspiro[2.4]heptane-5,7-dione | diazaspiro[2.4]heptane-5,7-dione |
| 4-[2-(methylphenylamino)carbonylpyridin)-4- | 4-(7-chloroquinolin)-4-ylmethyl-6-(4-trifluoromethane- |
| ylmethyl-6-(4-trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-[2-(4- | 4-(7-fluoroquinolin)-4-ylmethyl-6-(4-trifluoromethane- |
| methoxyphenyl)methylaminocarbonylpyridin)-4- | sulfanylphenyl)-4,6-diazaspiro[2.4]heptane-5,7-dione |
| ylmethyl-6-(4-trifluoromethanesulfonylphenyl)-4,6- | |
| diazaspiro[2.4]heptane-5,7-dione | |
| 4-[2-(4-chlorophenyl)methylaminocarbonylpyridin)- | 4-(7-aminoquinolin)-4-ylmethyl-6-(4-trifluoromethane- |
| 4-ylmethyl-6-(4-trifluoromethanesulfonylphenyl)- | sulfanylphenyl)-4,6-diazaspiro[2.4]heptane-5,7-dione |
| 4,6-diazaspiro[2.4]heptane-5,7-dione | |
| 4-[2-(4-morpholinylamino)carbonylpyridin)-4- | 4-(7-methylaminoquinolin)-4-ylmethyl-6-(4-trifluoro- |
| ylmethyl-6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-aminocarbonylpyridin)-4-ylmethyl-6-(4- | 4-(7-dimethylaminoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-phenoxypyrid-4-ylmethyl-6-(4-trifluoro- | 4-(7-piperidinoquinolin)-4-ylmethyl-6-(4-trifluoro- |
| methanesulfonylphenyl)-4,6-diazaspiro[2.4]heptane- | methanesulfanylphenyl)-4,6-diazaspiro[2.4]heptane- |
| 5,7-dione | 5,7-dione |
| 4-(2-methoxypyrid-4-ylmethyl-6-(4-trifluoro- | 4-(8-methylquinolin)-4-ylmethyl-6-(4- |
| methanesulfonylphenyl)-4,6-diazaspiro[2.4]heptane- | trifluoromethanesulfanylphenyl)-4,6- |
| 5,7-dione | diazaspiro[2.4]heptane-5,7-dione |
| 4-(2-ethoxypyrid-4-ylmethyl-6-(4-trifluoro- | 4-(8-methoxyquinolin)-4-ylmethyl-6-(4- |
| methanesulfonylphenyl)-4,6-diazaspiro[2.4]heptane- | trifluoromethanesulfanylphenyl)-4,6- |
| 5,7-dione | diazaspiro[2.4]heptane-5,7-dione |
| 4-[2-(1-pyrrolidinyl)ethoxypyrid-4-ylmethyl-6-(4- | 4-(8-chloroquinolin)-4-ylmethyl-6-(4-trifluoromethane- |
| trifluoromethanesulfonylphenyl)-4,6- | sulfanylphenyl)-4,6-diazaspiro[2.4]heptane-5,7-dione |
| diazaspiro[2.4]heptane-5,7-dione | |
| 4-(2-benzyloxypyrid-4-ylmethyl-6-(4-trifluoro- | 4-(8-fluoroquinolin)-4-ylmethyl-6-(4-trifluoromethane- |
| methanesulfonylphenyl)-4,6-diazaspiro[2.4]heptane- | sulfanylphenyl)-4,6-diazaspiro[2.4]heptane-5,7-dione |
| 5,7-dione | |
| 4-(2-benzoyloxypyrid-4-ylmethyl-6-(4-trifluoro- | 4-(8-aminoquinolin)-4-ylmethyl-6-(4-trifluoromethane- |
| methanesulfonylphenyl)-4,6-diazaspiro[2.4]heptane- | sulfanylphenyl)-4,6-diazaspiro[2.4]heptane-5,7-dione |
| 5,7-dione | |
| 4-[2-(4,5-dihydro-1H-imidazol-2-yl)pyridin]-4- | 4-(8-methylaminoquinolin)-4-ylmethyl-6-(4-trifluoro- |
| ylmethyl-6-(4-trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(1H-imidazol-2-yl)pyridin]-4-ylmethyl-6-(4- | 4-(8-dimethylaminoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-[2-(1H-tetrazol-5-yl)pyridin]-4-ylmethyl-6-(4- | 4-(8-piperidinoquinolin)-4-ylmethyl-6-(4-trifluoro- |
| trifluoromethanesulfonylphenyl)-4,6- | methanesulfanylphenyl)-4,6-diazaspiro[2.4]heptane- |
| diazaspiro[2.4]heptane-5,7-dione | 5,7-dione |
| 4-(2-amidinopyrid-4-ylmethyl-6-(4-trifluoro- | 5-methyl-1-(2-methylquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(2-dimethylaminomethylpyridin)-4-ylmethyl-6-(4- | 5-methyl-1-(2-methoxyquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-(2-aminomethylpyridin)-4-ylmethyl-6-(4-trifluoro- | 5-methyl-1-(2-chloroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-methylpyridin)-4-ylmethyl-6-(4-trifluoro- | 5-methyl-1-(2-fluoroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-ethylpyridin)-4-ylmethyl-6-(4-trifluoro- | 5-methyl-1-(2-aminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-isopropylpyridin)-4-ylmethyl-6-(4-trifluoro- | 5-methyl-1-(2-methylaminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-tert-butylpyridin)-4-ylmethyl-6-(4-trifluoro- | 5-methyl-1-(2-dimethylaminoquinolin-4-ylmethyl)-3- |
| methanesulfonylphenyl)-4,6-diazaspiro[2.4]heptane- | (4-trifluoromethanesulfanylphenyl)imidazolidine-2,4- |
| 5,7-dione | dione |
| 4-(3-isobutylpyridin)-4-ylmethyl-6-(4-trifluoro- | 5-methyl-1-(2-piperidinoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-cyclohexylpyridin)-4-ylmethyl-6-(4-trifluoro- | 5-methyl-1-(3-methylquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-phenylpyridin)-4-ylmethyl-6-(4-trifluoro- | 5-methyl-1-(3-methoxyquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-[3-(4-chlorophenyl)pyridin]-4-ylmethyl-6-(4- | 5-methyl-1-(3-chloroquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(3-chlorophenyl)pyridin]-4-ylmethyl-6-(4- | 5-methyl-1-(3-fluoroquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(2-chlorophenyl)pyridin]-4-ylmethyl-6-(4- | 5-methyl-1-(3-aminoquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(4-fluorophenyl)pyridin]-4-ylmethyl-6-(4- | 5-methyl-1-(3-methylaminoquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(4-methoxyphenyl)pyridin]-4-ylmethyl-6-(4- | 5-methyl-1-(3-dimethylaminoquinolin-4-ylmethyl)-3- |
| trifluoromethanesulfonylphenyl)-4,6- | (4-trifluoromethanesulfanylphenyl)imidazolidine-2,4- |
| diazaspiro[2.4]heptane-5,7-dione | dione |
| 4-(3-benzylpyridin)-4-ylmethyl-6-(4-trifluoro- | 5-methyl-1-(3-piperidinoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3,2′-bipyrid-4-ylmethyl-6-(4-trifluoro- | 5-methyl-1-(5-methylquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-hydroxymethylpyridin)-4-ylmethyl-6-(4- | 5-methyl-1-(5-methoxyquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-(3-chloropyrid-4-ylmethyl-6-(4-trifluoro- | 5-methyl-1-(5-chloroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-fluoropyrid-4-ylmethyl-6-(4-trifluoro- | 5-methyl-1-(5-fluoroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-bromopyrid-4-ylmethyl-6-(4-trifluoro- | 5-methyl-1-(5-aminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-aminopyrid-4-ylmethyl-6-(4-trifluoro- | 5-methyl-1-(5-methylaminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-methylaminopyrid-4-ylmethyl-6-(4-trifluoro- | 5-methyl-1-(5-dimethylaminoquinolin-4-ylmethyl)-3- |
| methanesulfonylphenyl)-4,6-diazaspiro[2.4]heptane- | (4-trifluoromethanesulfanylphenyl)imidazolidine-2,4- |
| 5,7-dione | dione |
| 4-(3-ethylaminopyrid-4-ylmethyl-6-(4-trifluoro- | 5-methyl-1-(5-piperidinoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-isopropylaminopyrid-4-ylmethyl-6-(4-trifluoro- | 5-methyl-1-(6-methylquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-phenylaminopyrid-4-ylmethyl-6-(4-trifluoro- | 5-methyl-1-(6-methoxyquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-cyclohexylaminopyrid-4-ylmethyl-6-(4- | 5-methyl-1-(6-chloroquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-(3-diethylaminopyrid-4-ylmethyl-6-(4-trifluoro- | 5-methyl-1-(6-fluoroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-N-cyclohexyl-N-methylaminopyrid-4- | 5-methyl-1-(6-aminoquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-(3-piperidinopyrid-4-ylmethyl-6-(4-trifluoro- | 5-methyl-1-(6-methylaminoquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-morpholinopyrid-4-ylmethyl-6-(4-trifluoro- | 5-methyl-1-(6-dimethylaminoquinolin-4-ylmethyl)-3- |
| methanesulfonylphenyl)-4,6-diazaspiro[2.4]heptane- | (4-trifluoromethanesulfanylphenyl)imidazolidine-2,4- |
| 5,7-dione | dione |
| 4-[3-(4-methylpiperazino)diethylaminopyrid-4- | 5-methyl-1-(6-piperidinoquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-[3-(1H-pyrazol-1-yl)pyridin]-4-ylmethyl-6-(4- | 5-methyl-1-(7-methylquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(2-benzoylhydrazino)pyrid-4-ylmethyl-6-(4- | 5-methyl-1-(7-methoxyquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-(3-benzoylaminopyrid-4-ylmethyl-6-(4-trifluoro- | 5-methyl-1-(7-chloroquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-[3-(4-chlorobenzoylamino)pyrid-4-ylmethyl-6-(4- | 5-methyl-1-(7-fluoroquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(3-chlorobenzoylamino)pyrid-4-ylmethyl-6-(4- | 5-methyl-1-(7-aminoquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(2-chlorobenzoylamino)pyrid-4-ylmethyl-6-(4- | 5-methyl-1-(7-methylaminoquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(4-fluorobenzoylamino)pyrid-4-ylmethyl-6-(4- | 5-methyl-1-(7-dimethylaminoquinolin-4-ylmethyl)-3- |
| trifluoromethanesulfonylphenyl)-4,6- | (4-trifluoromethanesulfanylphenyl)imidazolidine-2,4- |
| diazaspiro[2.4]heptane-5,7-dione | dione |
| 4-[3-(4-methoxybenzoylamino)pyrid-4-ylmethyl-6- | 5-methyl-1-(7-piperidinoquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-(3-acetamidopyrid-4-ylmethyl-6-(4-trifluoro- | 5-methyl-1-(8-methylquinolin-4-ylmethyl)-3-(4- |
| methanesulfonylphenyl)-4,6-diazaspiro[2.4]heptane- | trifluoromethanesulfanylphenyl)imidazolidine-2,4- |
| 5,7-dione | dione |
| 4-(3-phenylacetamidopyrid-4-ylmethyl-6-(4- | 5-methyl-1-(8-methoxyquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(4-methylpiperazino)acetamidopyrid-4- | 5-methyl-1-(8-chloroquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-(3-cyclohexanylcarboxamidopyrid-4-ylmethyl-6- | 5-methyl-1-(8-fluoroquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-[3-(1-methyl-4-piperidinyl)carbonylaminopyrid-4- | 5-methyl-1-(8-aminoquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-[3-(2-furanylcarbonyl)aminopyrid-4-ylmethyl-6- | 5-methyl-1-(8-methylaminoquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-(3-ethoxycarbonylaminopyrid-4-ylmethyl-6-(4- | 5-methyl-1-(8-dimethylaminoquinolin-4-ylmethyl)-3- |
| trifluoromethanesulfonylphenyl)-4,6- | (4-trifluoromethanesulfanylphenyl)imidazolidine-2,4- |
| diazaspiro[2.4]heptane-5,7-dione | dione |
| 4-(3-isopropoxycarbonylaminopyrid-4-ylmethyl-6- | 5-methyl-1-(8-piperidinoquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-(3-butoxycarbonylaminopyrid-4-ylmethyl-6-(4- | 1-(2-methylquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(2-diethylaminoethoxycarbonylamino)pyrid-4- | 1-(2-methoxyquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-(3-phenoxycarbonylaminopyrid-4-ylmethyl-6-(4- | 1-(2-chloroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-[3-(4-chloroanilino)carbonylaminopyrid-4- | 1-(2-fluoroquinolin-4-ylmethyl)-3-(4-trifluoromethane- |
| ylmethyl)-6-(4-trifluoromethanesulfonylphenyl)-4,6- | sulfanylphenyl)imidazolidine-2,4-dione |
| diazaspiro[2.4]heptane-5,7-dione | |
| 4-(3-anilinocarbonylaminopyrid-4-ylmethyl-6-(4- | 1-(2-aminoquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-benzylaminocarbonylaminopyrid-4-ylmethyl-6- | 1-(2-methylaminoquinolin-4-ylmethyl)-3-(4-trifluoro- |
| (4-trifluoromethanesulfonylphenyl)-4,6- | methanesulfanylphenyl)imidazolidine-2,4-dione |
| diazaspiro[2.4]heptane-5,7-dione | |
| 4-[3-(2-pyridylamino)carbonylaminopyrid-4- | 1-(2-dimethylaminoquinolin-4-ylmethyl)-3-(4-trifluoro- |
| ylmethyl)-6-(4-trifluoromethanesulfonylphenyl)-4,6- | methanesulfanylphenyl)imidazolidine-2,4-dione |
| diazaspiro[2.4]heptane-5,7-dione | |
| 4-(3-ureidopyrid-4-ylmethyl-6-(4-trifluoro- | 1-(2-piperidinoquinolin-4-ylmethyl)-3-(4-trifluoro- |
| methanesulfonylphenyl)-4,6-diazaspiro[2.4]heptane- | methanesulfanylphenyl)imidazolidine-2,4-dione |
| 5,7-dione | |
| 4-(3-benzylaminosulfonylaminopyrid-4-ylmethyl-6- | 1-(3-methylquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-(3-methylaminosulfonylaminopyrid-4-ylmethyl-6- | 1-(3-methoxyquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-(3-aminosulfonylaminopyrid-4-ylmethyl-6-(4- | 1-(3-chloroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-morpholinosulfonylaminopyrid-4-ylmethyl-6- | 1-(3-fluoroquinolin-4-ylmethyl)-3-(4-trifluoromethane- |
| (4-trifluoromethanesulfonylphenyl)-4,6- | sulfanylphenyl)imidazolidine-2,4-dione |
| diazaspiro[2.4]heptane-5,7-dione | |
| 4-(3-dimethylaminosulfonylaminopyrid-4-ylmethyl- | 1-(3-aminoquinolin-4-ylmethyl)-3-(4-trifluoromethane- |
| 6-(4-trifluoromethanesulfonylphenyl)-4,6- | sulfanylphenyl)imidazolidine-2,4-dione |
| diazaspiro[2.4]heptane-5,7-dione | |
| 4-(3-anilinosulfonylaminopyrid-4-ylmethyl-6-(4- | 1-(3-methylaminoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-(3-phenylsulfonylaminopyrid-4-ylmethyl-6-(4- | 1-(3-dimethylaminoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-[3-(2-thienyl)sulfonylaminopyrid-4-ylmethyl-6-(4- | 1-(3-piperidinoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-(3-methylsulfonylaminopyrid-4-ylmethyl-6-(4- | 1-(5-methylquinolin-4-ylmethyl)-3-(4- |
| trifluoromethanesulfonylphenyl)-4,6- | trifluoromethanesulfanylphenyl)imidazolidine-2,4- |
| diazaspiro[2.4]heptane-5,7-dione | dione |
| 4-[3-(4-chlorophenyl)sulfonylaminopyrid-4- | 1-(5-methoxyquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-(3-benzylsulfonylaminopyrid-4-ylmethyl-6-(4- | 1-(5-chloroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-hydroxycarbonylpyridin)-4-ylmethyl-6-(4- | 1-(5-fluoroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-ethoxycarbonylpyridin)-4-ylmethyl-6-(4- | 1-(5-aminoquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-tert-butoxycarbonylpyridin)-4-ylmethyl-6-(4- | 1-(5-methylaminoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-(3-phenoxycarbonylpyridin)-4-ylmethyl-6-(4- | 1-(5-dimethylaminoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-[3-(4-methylpiperazinocarbonyl)pyridin]-4- | 1-(5-piperidinoquinolin-4-ylmethyl)-3-(4-trifluoro- |
| ylmethyl-6-(4-trifluoromethanesulfonylphenyl)-4,6- | methanesulfanylphenyl)imidazolidine-2,4-dione |
| diazaspiro[2.4]heptane-5,7-dione | |
| 4-[3-(methylamino)carbonylpyridin)-4-ylmethyl-6- | 1-(6-methylquinolin-4-ylmethyl)-3-(4- |
| (4-trifluoromethanesulfonylphenyl)-4,6- | trifluoromethanesulfanylphenyl)imidazolidine-2,4- |
| diazaspiro[2.4]heptane-5,7-dione | dione |
| 4-[3-(dimethylamino)carbonylpyridin)-4-ylmethyl- | 1-(6-methoxyquinolin-4-ylmethyl)-3-(4- |
| 6-(4-trifluoromethanesulfonylphenyl)-4,6- | trifluoromethanesulfanylphenyl)imidazolidine-2,4- |
| diazaspiro[2.4]heptane-5,7-dione | dione |
| 4-(3-piperidinocarbonylpyridin)-4-ylmethyl-6-(4- | 1-(6-chloroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-morpholinocarbonylpyridin)-4-ylmethyl-6-(4- | 1-(6-fluoroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-[3-(4-phenylpiperazinocarbonyl)pyridin]-4- | 1-(6-aminoquinolin-4-ylmethyl)-3-(4-trifluoromethane- |
| ylmethyl-6-(4-trifluoromethanesulfonylphenyl)-4,6- | sulfanylphenyl)imidazolidine-2,4-dione |
| diazaspiro[2.4]heptane-5,7-dione | |
| 4-(3-phenylaminocarbonylpyridin)-4-ylmethyl-6-(4- | 1-(6-methylaminoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-[3-(methylphenylamino)carbonylpyridin)-4- | 1-(6-dimethylaminoquinolin-4-ylmethyl)-3-(4-trifluoro- |
| ylmethyl-6-(4-trifluoromethanesulfonylphenyl)-4,6- | methanesulfanylphenyl)imidazolidine-2,4-dione |
| diazaspiro[2.4]heptane-5,7-dione | |
| 4-[3-(4- | 1-(6-piperidinoquinolin-4-ylmethyl)-3-(4-trifluoro- |
| methoxyphenyl)methylaminocarbonylpyridin)-4- | methanesulfanylphenyl)imidazolidine-2,4-dione |
| ylmethyl-6-(4-trifluoromethanesulfonylphenyl)-4,6- | |
| diazaspiro[2.4]heptane-5,7-dione | |
| 4-[3-(4-chlorophenyl)methylaminocarbonylpyridin)- | 1-(7-methylquinolin-4-ylmethyl)-3-(4- |
| 4-ylmethyl-6-(4-trifluoromethanesulfonylphenyl)- | trifluoromethanesulfanylphenyl)imidazolidine-2,4- |
| 4,6-diazaspiro[2.4]heptane-5,7-dione | dione |
| 4-[3-(4-morpholinylamino)carbonylpyridin)-4- | 1-(7-methoxyquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-(3-aminocarbonylpyridin)-4-ylmethyl-6-(4- | 1-(7-chloroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-phenoxypyrid-4-ylmethyl-6-(4-trifluoro- | 1-(7-fluoroquinolin-4-ylmethyl)-3-(4-trifluoromethane- |
| methanesulfonylphenyl)-4,6-diazaspiro[2.4]heptane- | sulfanylphenyl)imidazolidine-2,4-dione |
| 5,7-dione | |
| 4-(3-methoxypyrid-4-ylmethyl-6-(4-trifluoro- | 1-(7-aminoquinolin-4-ylmethyl)-3-(4-trifluoromethane- |
| methanesulfonylphenyl)-4,6-diazaspiro[2.4]heptane- | sulfanylphenyl)imidazolidine-2,4-dione |
| 5,7-dione | |
| 4-(3-ethoxypyrid-4-ylmethyl-6-(4-trifluoro- | 1-(7-methylaminoquinolin-4-ylmethyl)-3-(4-trifluoro- |
| methanesulfonylphenyl)-4,6-diazaspiro[2.4]heptane- | methanesulfanylphenyl)imidazolidine-2,4-dione |
| 5,7-dione | |
| 4-[3-(1-pyrrolidinyl)ethoxypyrid-4-ylmethyl-6-(4- | 1-(7-dimethylaminoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-(3-benzyloxypyrid-4-ylmethyl-6-(4-trifluoro- | 1-(7-piperidinoquinolin-4-ylmethyl)-3-(4-trifluoro- |
| methanesulfonylphenyl)-4,6-diazaspiro[2.4]heptane- | methanesulfanylphenyl)imidazolidine-2,4-dione |
| 5,7-dione | |
| 4-(3-benzoyloxypyrid-4-ylmethyl-6-(4-trifluoro- | 1-(8-methylquinolin-4-ylmethyl)-3-(4-trifluoro- |
| methanesulfonylphenyl)-4,6-diazaspiro[2.4]heptane- | methanesulfanylphenyl)imidazolidine-2,4-dione |
| 5,7-dione | |
| 4-[3-(4,5-dihydro-1H-imidazol-2-yl)pyridin]-4- | 1-(8-methoxyquinolin-4-ylmethyl)-3-(4- |
| ylmethyl-6-(4-trifluoromethanesulfonylphenyl)-4,6- | trifluoromethanesulfanylphenyl)imidazolidine-2,4- |
| diazaspiro[2.4]heptane-5,7-dione | dione |
| 4-[3-(1H-imidazol-2-yl)pyridin]-4-ylmethyl-6-(4- | 1-(8-chloroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-[3-(1H-tetrazol-5-yl)pyridin]-4-ylmethyl-6-(4- | 1-(8-fluoroquinolin-4-ylmethyl)-3-(4-trifluoromethane- |
| trifluoromethanesulfonylphenyl)-4,6- | sulfanylphenyl)imidazolidine-2,4-dione |
| diazaspiro[2.4]heptane-5,7-dione | |
| 4-(3-amidinopyrid-4-ylmethyl-6-(4-trifluoro- | 1-(8-aminoquinolin-4-ylmethyl)-3-(4-trifluoromethane- |
| methanesulfonylphenyl)-4,6-diazaspiro[2.4]heptane- | sulfanylphenyl)imidazolidine-2,4-dione |
| 5,7-dione | |
| 4-(3-dimethylaminomethylpyridin)-4-ylmethyl-6-(4- | 1-(8-methylaminoquinolin-4-ylmethyl)-3-(4-trifluoro- |
| trifluoromethanesulfonylphenyl)-4,6- | methanesulfanylphenyl)imidazolidine-2,4-dione |
| diazaspiro[2.4]heptane-5,7-dione | |
| 4-(3-aminomethylpyridin)-4-ylmethyl-6-(4-trifluoro- | 1-(8-dimethylaminoquinolin-4-ylmethyl)-3-(4-trifluoro- |
| methanesulfonylphenyl)-4,6-diazaspiro[2.4]heptane- | methanesulfanylphenyl)imidazolidine-2,4-dione |
| 5,7-dione | |
| 5-methyl-1-(2-methylpyrid-4-ylmethyl)-3-(4- | 1-(8-piperidinoquinolin-4-ylmethyl)-3-(4-trifluoro- |
| trifluoromethanesulfonylphenyl)imidazolidine-2,4- | methanesulfanylphenyl)imidazolidine-2,4-dione |
| dione | |
| 5-methyl-1-(2-ethylpyrid-4-ylmethyl)-3-(4-trifluoro- | 5,5-dimethyl-1-(2-methylquinolin-4-ylmethyl)-3-(4- |
| methanesulfonylphenyl)imidazolidine-2,4-dione | trifluoromethanesulfonylphenyl)imidazolidine-2,4- |
| | dione |
| 5-methyl-1-(2-isopropylpyrid-4-ylmethyl)-3-(4- | 5,5-dimethyl-1-(2-methoxyquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-tert-butylpyrid-4-ylmethyl)-3-(4- | 5,5-dimethyl-1-(2-chloroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-isobutylpyrid-4-ylmethyl)-3-(4- | 5,5-dimethyl-1-(2-fluoroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-cyclohexylpyrid-4-ylmethyl)-3-(4- | 5,5-dimethyl-1-(2-aminoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-phenylpyrid-4-ylmethyl)-3-(4- | 5,5-dimethyl-1-(2-methylaminoquinolin-4-ylmethyl)-3- |
| trifluoromethanesulfonylphenyl)imidazolidine-2,4- | (4-trifluoromethanesulfonylphenyl)imidazolidine-2,4- |
| dione | dione |
| 5-methyl-1-[2-(4-chlorophenyl)pyrid-4-ylmethyl]-3- | 5,5-dimethyl-1-(2-dimethylaminoquinolin-4-ylmethyl)- |
| (4-trifluoromethanesulfonylphenyl)imidazolidine- | 3-(4-trifluoromethanesulfonylphenyl)imidazolidine-2,4- |
| 2,4-dione | dione |
| 5-methyl-1-[2-(3-chlorophenyl)pyrid-4-ylmethyl]-3- | 5,5-dimethyl-1-(2-piperidinoquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[2-(2-chlorophenyl)pyrid-4-ylmethyl]-3- | 5,5-dimethyl-1-(3-methylquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[2-(4-fluorophenyl)pyrid-4-ylmethyl]-3- | 5,5-dimethyl-1-(3-methoxyquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[2-(4-methoxyphenyl)pyrid-4-ylmethyl]- | 5,5-dimethyl-1-(3-chloroquinolin-4-ylmethyl)-3-(4- |
| 3-(4-trifluoromethanesulfonylphenyl)imidazolidine- | trifluoromethanesulfonylphenyl)imidazolidine-2,4- |
| 2,4-dione | dione |
| 5-methyl-1-(2-benzylpyrid-4-ylmethyl)-3-(4- | 5,5-dimethyl-1-(3-fluoroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2,2′-bipyrid-4-ylmethyl)-3-(4-trifluoro- | 5,5-dimethyl-1-(3-aminoquinolin-4-ylmethyl)-3-(4- |
| methanesulfonylphenyl)imidazolidine-2,4-dione | trifluoromethanesulfonylphenyl)imidazolidine-2,4- |
| | dione |
| 5-methyl-1-(2-hydroxymethylpyrid-4-ylmethyl)-3- | 5,5-dimethyl-1-(3-methylaminoquinolin-4-ylmethyl)-3- |
| (4-trifluoromethanesulfonylphenyl)imidazolidine- | (4-trifluoromethanesulfonylphenyl)imidazolidine-2,4- |
| 2,4-dione | dione |
| 5-methyl-1-(2-chloropyrid-4-ylmethyl)-3-(4- | 5,5-dimethyl-1-(3-dimethylaminoquinolin-4-ylmethyl)- |
| trifluoromethanesulfonylphenyl)imidazolidine-2,4- | 3-(4-trifluoromethanesulfonylphenyl)imidazolidine-2,4- |
| dione | dione |
| 5-methyl-1-(2-fluoropyrid-4-ylmethyl)-3-(4- | 5,5-dimethyl-1-(3-piperidinoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-bromopyrid-4-ylmethyl)-3-(4- | 5,5-dimethyl-1-(5-methylquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-aminopyrid-4-ylmethyl)-3-(4- | 5,5-dimethyl-1-(5-methoxyquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-methylaminopyrid-4-ylmethyl)-3-(4- | 5,5-dimethyl-1-(5-chloroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-ethylaminopyrid-4-ylmethyl)-3-(4- | 5,5-dimethyl-1-(5-fluoroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-isopropylaminopyrid-4-ylmethyl)-3- | 5,5-dimethyl-1-(5-aminoquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-(2-phenylaminopyrid-4-ylmethyl)-3-(4- | 5,5-dimethyl-1-(5-methylaminoquinolin-4-ylmethyl)-3- |
| trifluoromethanesulfonylphenyl)imidazolidine-2,4- | (4-trifluoromethanesulfonylphenyl)imidazolidine-2,4- |
| dione | dione |
| 5-methyl-1-(2-cyclohexylaminopyrid-4-ylmethyl)-3- | 5,5-dimethyl-1-(5-dimethylaminoquinolin-4-ylmethyl)- |
| (4-trifluoromethanesulfonylphenyl)imidazolidine- | 3-(4-trifluoromethanesulfonylphenyl)imidazolidine-2,4- |
| 2,4-dione | dione |
| 5-methyl-1-(2-diethylaminopyrid-4-ylmethyl)-3-(4- | 5,5-dimethyl-1-(5-piperidinoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-N-cyclohexyl-N-methylaminopyrid- | 5,5-dimethyl-1-(6-methylquinolin-4-ylmethyl)-3-(4- |
| 4-ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(2-piperidinopyrid-4-ylmethyl)-3-(4- | 5,5-dimethyl-1-(6-methoxyquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-morpholinopyrid-4-ylmethyl)-3-(4- | 5,5-dimethyl-1-(6-chloroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-[2-(4- | 5,5-dimethyl-1-(6-fluoroquinolin-4-ylmethyl)-3-(4- |
| methylpiperazino)diethylaminopyrid-4-ylmethyl]-3- | trifluoromethanesulfonylphenyl)imidazolidine-2,4- |
| (4-trifluoromethanesulfonylphenyl)imidazolidine- | dione |
| 2,4-dione | |
| 5-methyl-1-[2-(1H-pyrazol-1-yl)pyrid-4-ylmethyl]- | 5,5-dimethyl-1-(6-aminoquinolin-4-ylmethyl)-3-(4- |
| 3-(4-trifluoromethanesulfonylphenyl)imidazolidine- | trifluoromethanesulfonylphenyl)imidazolidine-2,4- |
| 2,4-dione | dione |
| 5-methyl-1-[2-(2-benzoylhydrazino)pyrid-4- | 5,5-dimethyl-1-(6-methylaminoquinolin-4-ylmethyl)-3- |
| ylmethyl]-3-(4-trifluoro- | (4-trifluoromethanesulfonylphenyl)imidazolidine-2,4- |
| methanesulfonylphenyl)imidazolidine-2,4-dione | dione |
| 5-methyl-1-(2-benzoylaminopyrid-4-ylmethyl)-3-(4- | 5,5-dimethyl-1-(6-dimethylaminoquinolin-4-ylmethyl)- |
| trifluoromethanesulfonylphenyl)imidazolidine-2,4- | 3-(4-trifluoromethanesulfonylphenyl)imidazolidine-2,4- |
| dione | dione |
| 5-methyl-1-[2-(4-chlorobenzoylamino)pyrid-4- | 5,5-dimethyl-1-(6-piperidinoquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[2-(3-chlorobenzoylamino)pyrid-4- | 5,5-dimethyl-1-(7-methylquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[2-(2-chlorobenzoylamino)pyrid-4- | 5,5-dimethyl-1-(7-methoxyquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[2-(4-fluorobenzoylamino)pyrid-4- | 5,5-dimethyl-1-(7-chloroquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[2-(4-methoxybenzoylamino)pyrid-4- | 5,5-dimethyl-1-(7-fluoroquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(2-acetamidopyrid-4-ylmethyl)-3-(4- | 5,5-dimethyl-1-(7-aminoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(2-phenylacetamidopyrid-4-ylmethyl)-3- | 5,5-dimethyl-1-(7-methylaminoquinolin-4-ylmethyl)-3- |
| (4-trifluoromethanesulfonylphenyl)imidazolidine- | (4-trifluoromethanesulfonylphenyl)imidazolidine-2,4- |
| 2,4-dione | dione |
| 5-methyl-1-[2-(4-methylpiperazino)acetamidopyrid- | 5,5-dimethyl-1-(7-dimethylaminoquinolin-4-ylmethyl)- |
| 4-ylmethyl]-3-(4-trifluoro- | 3-(4-trifluoromethanesulfonylphenyl)imidazolidine-2,4- |
| methanesulfonylphenyl)imidazolidine-2,4-dione | dione |
| 5-methyl-1-(2-cyclohexanylcarboxamidopyrid-4- | 5,5-dimethyl-1-(7-piperidinoquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[2-(1-methyl-4- | 5,5-dimethyl-1-(8-methylquinolin-4-ylmethyl)-3-(4- |
| piperidinyl)carbonylaminopyrid-4-ylmethyl]-3-(4- | trifluoromethanesulfonylphenyl)imidazolidine-2,4- |
| trifluoromethanesulfonylphenyl)imidazolidine-2,4- | dione |
| dione | |
| 5-methyl-1-[2-(2-furanylcarbonyl)aminopyrid-4- | 5,5-dimethyl-1-(8-methoxyquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(2-ethoxycarbonylaminopyrid-4- | 5,5-dimethyl-1-(8-chloroquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(2-isopropoxycarbonylaminopyrid-4- | 5,5-dimethyl-1-(8-fluoroquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(2-butoxycarbonylaminopyrid-4- | 5,5-dimethyl-1-(8-aminoquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[2-(2- | 5,5-dimethyl-1-(8-methylaminoquinolin-4-ylmethyl)-3- |
| diethylaminoethoxycarbonylamino)pyrid-4- | (4-trifluoromethanesulfonylphenyl)imidazolidine-2,4- |
| ylmethyl]-3-(4-trifluoro- | dione |
| methanesulfonylphenyl)imidazolidine-2,4-dione | |
| 5-methyl-1-(2-phenoxycarbonylaminopyrid-4- | 5,5-dimethyl-1-(8-dimethylaminoquinolin-4-ylmethyl)- |
| ylmethyl)-3-(4-trifluoro- | 3-(4-trifluoromethanesulfonylphenyl)imidazolidine-2,4- |
| methanesulfonylphenyl)imidazolidine-2,4-dione | dione |
| 5-methyl-1-[2-(4-chloroanilino)carbonylaminopyrid- | 5,5-dimethyl-1-(8-piperidinoquinolin-4-ylmethyl)-3-(4- |
| 4-ylmethyl)]-3-(4-trifluoro- | trifluoromethanesulfonylphenyl)imidazolidine-2,4- |
| methanesulfonylphenyl)imidazolidine-2,4-dione | dione |
| 5-methyl-1-(2-anilinocarbonylaminopyrid-4- | 4-(2-methylquinolin)-4-ylmethyl-6-(4- |
| ylmethyl)-3-(4-trifluoro- | trifluoromethanesulfonylphenyl)-4,6- |
| methanesulfonylphenyl)imidazolidine-2,4-dione | diazaspiro[2.4]heptane-5,7-dione |
| 5-methyl-1-(2-benzylaminocarbonylaminopyrid-4- | 4-(2-methoxyquinolin)-4-ylmethyl-6-(4- |
| ylmethyl)-3-(4-trifluoro- | trifluoromethanesulfonylphenyl)-4,6- |
| methanesulfonylphenyl)imidazolidine-2,4-dione | diazaspiro[2.4]heptane-5,7-dione |
| 5-methyl-1-[2-(2- | 4-(2-chloroquinolin)-4-ylmethyl-6-(4-trifluoromethane- |
| pyridylamino)carbonylaminopyrid-4-ylmethyl)]-3- | sulfonylphenyl)-4,6-diazaspiro[2.4]heptane-5,7-dione |
| (4-trifluoromethanesulfonylphenyl)imidazolidine- | |
| 2,4-dione | |
| 5-methyl-1-(2-ureidopyrid-4-ylmethyl)-3-(4- | 4-(2-fluoroquinolin)-4-ylmethyl-6-(4-trifluoromethane- |
| trifluoromethanesulfonylphenyl)imidazolidine-2,4- | sulfonylphenyl)-4,6-diazaspiro[2.4]heptane-5,7-dione |
| dione | |
| 5-methyl-1-(2-benzylaminosulfonylaminopyrid-4- | 4-(2-aminoquinolin)-4-ylmethyl-6-(4-trifluoromethane- |
| ylmethyl)-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-(2-methylaminosulfonylaminopyrid-4- | 4-(2-methylaminoquinolin)-4-ylmethyl-6-(4-trifluoro- |
| ylmethyl)-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-(2-aminosulfonylaminopyrid-4- | 4-(2-dimethylaminoquinolin)-4-ylmethyl-6-(4-trifluoro- |
| ylmethyl)-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-(2-morpholinosulfonylaminopyrid-4- | 4-(2-piperidinoquinolin)-4-ylmethyl-6-(4-trifluoro- |
| ylmethyl)-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-(2-dimethylaminosulfonylaminopyrid-4- | 4-(3-methylquinolin)-4-ylmethyl-6-(4- |
| ylmethyl)-3-(4-trifluoro- | trifluoromethanesulfonylphenyl)-4,6- |
| methanesulfonylphenyl)imidazolidine-2,4-dione | diazaspiro[2.4]heptane-5,7-dione |
| 5-methyl-1-(2-anilinosulfonylaminopyrid-4- | 4-(3-methoxyquinolin)-4-ylmethyl-6-(4- |
| ylmethyl)-3-(4-trifluoro- | trifluoromethanesulfonylphenyl)-4,6- |
| methanesulfonylphenyl)imidazolidine-2,4-dione | diazaspiro[2.4]heptane-5,7-dione |
| 5-methyl-1-(2-phenylsulfonylaminopyrid-4- | 4-(3-chloroquinolin)-4-ylmethyl-6-(4-trifluoromethane- |
| ylmethyl)-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-[2-(2-thienyl)sulfonylaminopyrid-4- | 4-(3-fluoroquinolin)-4-ylmethyl-6-(4-trifluoromethane- |
| ylmethyl]-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-(2-methylsulfonylaminopyrid-4- | 4-(3-aminoquinolin)-4-ylmethyl-6-(4-trifluoromethane- |
| ylmethyl)-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-[2-(4-chlorophenyl)sulfonylaminopyrid- | 4-(3-methylaminoquinolin)-4-ylmethyl-6-(4-trifluoro- |
| 4-ylmethyl]-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-(2-benzylsulfonylaminopyrid-4- | 4-(3-dimethylaminoquinolin)-4-ylmethyl-6-(4-trifluoro- |
| ylmethyl)-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-(2-hydroxycarbonylpyrid-4-ylmethyl)-3- | 4-(3-piperidinoquinolin)-4-ylmethyl-6-(4-trifluoro- |
| (4-trifluoromethanesulfonylphenyl)imidazolidine- | methanesulfonylphenyl)-4,6-diazaspiro[2.4]heptane- |
| 2,4-dione | 5,7-dione |
| 5-methyl-1-(2-ethoxycarbonylpyrid-4-ylmethyl)-3- | 4-(5-methylquinolin)-4-ylmethyl-6-(4- |
| (4-trifluoromethanesulfonylphenyl)imidazolidine- | trifluoromethanesulfonylphenyl)-4,6- |
| 2,4-dione | diazaspiro[2.4]heptane-5,7-dione |
| 5-methyl-1-(2-tert-butoxycarbonylpyrid-4- | 4-(5-methoxyquinolin)-4-ylmethyl-6-(4- |
| ylmethyl)-3-(4-trifluoro- | trifluoromethanesulfonylphenyl)-4,6- |
| methanesulfonylphenyl)imidazolidine-2,4-dione | diazaspiro[2.4]heptane-5,7-dione |
| 5-methyl-1-(2-phenoxycarbonylpyrid-4-ylmethyl)-3- | 4-(5-chloroquinolin)-4-ylmethyl-6-(4-trifluoromethane- |
| (4-trifluoromethanesulfonylphenyl)imidazolidine- | sulfonylphenyl)-4,6-diazaspiro[2.4]heptane-5,7-dione |
| 2,4-dione | |
| 5-methyl-1-[2-(4-methylpiperazinocarbonyl)pyrid-4- | 4-(5-fluoroquinolin)-4-ylmethyl-6-(4-trifluoromethane- |
| ylmethyl]-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-[2-(methylamino)carbonylpyrid-4- | 4-(5-aminoquinolin)-4-ylmethyl-6-(4-trifluoromethane- |
| ylmethyl]-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-[2-(dimethylamino)carbonylpyrid-4- | 4-(5-methylaminoquinolin)-4-ylmethyl-6-(4-trifluoro- |
| ylmethyl]-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-(2-piperidinocarbonylpyrid-4-ylmethyl)- | 4-(5-dimethylaminoquinolin)-4-ylmethyl-6-(4-trifluoro- |
| 3-(4-trifluoromethanesulfonylphenyl)imidazolidine- | methanesulfonylphenyl)-4,6-diazaspiro[2.4]heptane- |
| 2,4-dione | 5,7-dione |
| 5-methyl-1-(2-morpholinocarbonylpyrid-4- | 4-(5-piperidinoquinolin)-4-ylmethyl-6-(4-trifluoro- |
| ylmethyl)-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-[2-(4-phenylpiperazinocarbonyl)pyrid-4- | 4-(6-methylquinolin)-4-ylmethyl-6-(4- |
| ylmethyl]-3-(4-trifluoro- | trifluoromethanesulfonylphenyl)-4,6- |
| methanesulfonylphenyl)imidazolidine-2,4-dione | diazaspiro[2.4]heptane-5,7-dione |
| 5-methyl-1-(2-phenylaminocarbonylpyrid-4- | 4-(6-methoxyquinolin)-4-ylmethyl-6-(4-trifluoro- |
| ylmethyl)-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-[2-(methylphenylamino)carbonylpyrid- | 4-(6-chloroquinolin)-4-ylmethyl-6-(4-trifluoromethane- |
| 4-ylmethyl]-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-[2-(4- | 4-(6-fluoroquinolin)-4-ylmethyl-6-(4-trifluoromethane- |
| methoxyphenyl)methylaminocarbonylpyrid-4- | sulfonylphenyl)-4,6-diazaspiro[2.4]heptane-5,7-dione |
| ylmethyl]-3-(4-trifluoro- | |
| methanesulfonylphenyl)imidazolidine-2,4-dione | |
| 5-methyl-1-[2-(4- | 4-(6-aminoquinolin)-4-ylmethyl-6-(4-trifluoromethane- |
| chlorophenyl)methylaminocarbonylpyrid-4- | sulfonylphenyl)-4,6-diazaspiro[2.4]heptane-5,7-dione |
| ylmethyl]-3-(4-trifluoro- | |
| methanesulfonylphenyl)imidazolidine-2,4-dione | |
| 5-methyl-1-[2-(4-morpholinylamino)carbonylpyrid- | 4-(6-methylaminoquinolin)-4-ylmethyl-6-(4-trifluoro- |
| 4-ylmethyl]-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-(2-aminocarbonylpyrid-4-ylmethyl)-3- | 4-(6-dimethylaminoquinolin)-4-ylmethyl-6-(4-trifluoro- |
| (4-trifluoromethanesulfonylphenyl)imidazolidine- | methanesulfonylphenyl)-4,6-diazaspiro[2.4]heptane- |
| 2,4-dione | 5,7-dione |
| 5-methyl-1-(2-phenoxypyrid-4-ylmethyl)-3-(4- | 4-(6-piperidinoquinolin)-4-ylmethyl-6-(4-trifluoro- |
| trifluoromethanesulfonylphenyl)imidazolidine-2,4- | methanesulfonylphenyl)-4,6-diazaspiro[2.4]heptane- |
| dione | 5,7-dione |
| 5-methyl-1-(2-methoxypyrid-4-ylmethyl)-3-(4- | 4-(7-methylquinolin)-4-ylmethyl-6-(4- |
| trifluoromethanesulfonylphenyl)imidazolidine-2,4- | trifluoromethanesulfonylphenyl)-4,6- |
| dione | diazaspiro[2.4]heptane-5,7-dione |
| 5-methyl-1-(2-ethoxypyrid-4-ylmethyl)-3-(4- | 4-(7-methoxyquinolin)-4-ylmethyl-6-(4- |
| trifluoromethanesulfonylphenyl)imidazolidine-2,4- | trifluoromethanesulfonylphenyl)-4,6- |
| dione | diazaspiro[2.4]heptane-5,7-dione |
| 5-methyl-1-[2-(1-pyrrolidinyl)ethoxypyrid-4- | 4-(7-chloroquinolin)-4-ylmethyl-6-(4-trifluoromethane- |
| ylmethyl]-3-(4-trifluoro- | sulfonylphenyl)-4,6-diazaspiro[2.4]heptane-5,7-dione |
| methanesulfonylphenyl)imidazolidine-2,4-dione | |
| 5-methyl-1-(2-benzyloxypyrid-4-ylmethyl)-3-(4- | 4-(7-fluoroquinolin)-4-ylmethyl-6-(4-trifluoromethane- |
| trifluoromethanesulfonylphenyl)imidazolidine-2,4- | sulfonylphenyl)-4,6-diazaspiro[2.4]heptane-5,7-dione |
| dione | |
| 5-methyl-1-(2-benzoyloxypyrid-4-ylmethyl)-3-(4- | 4-(7-aminoquinolin)-4-ylmethyl-6-(4-trifluoromethane- |
| trifluoromethanesulfonylphenyl)imidazolidine-2,4- | sulfonylphenyl)-4,6-diazaspiro[2.4]heptane-5,7-dione |
| dione | |
| 5-methyl-1-[2-(4,5-dihydro-1H-imidazol-2-yl)pyrid- | 4-(7-methylaminoquinolin)-4-ylmethyl-6-(4-trifluoro- |
| 4-ylmethyl]-3-(4-trifluoro- | methanesulfonylphenyl)-4,6-diazaspiro[2.4]heptane- |
| methanesulfonylphenyl)imidazolidine-2,4-dione | 5,7-dione |
| 5-methyl-1-[2-(1H-imidazol-2-yl)pyrid-4-ylmethyl]- | 4-(7-dimethylaminoquinolin)-4-ylmethyl-6-(4-trifluoro- |
| 3-(4-trifluoromethanesulfonylphenyl)imidazolidine- | methanesulfonylphenyl)-4,6-diazaspiro[2.4]heptane- |
| 2,4-dione | 5,7-dione |
| 5-methyl-1-[2-(1H-tetrazol-5-yl)pyrid-4-ylmethyl]- | 4-(7-piperidinoquinolin)-4-ylmethyl-6-(4-trifluoro- |
| 3-(4-trifluoromethanesulfonylphenyl)imidazolidine- | methanesulfonylphenyl)-4,6-diazaspiro[2.4]heptane- |
| 2,4-dione | 5,7-dione |
| 5-methyl-1-(2-amidinopyrid-4-ylmethyl)-3-(4- | 4-(8-methylquinolin)-4-ylmethyl-6-(4- |
| trifluoromethanesulfonylphenyl)imidazolidine-2,4- | trifluoromethanesulfonylphenyl)-4,6- |
| dione | diazaspiro[2.4]heptane-5,7-dione |
| 5-methyl-1-(2-dimethylaminomethylpyrid-4- | 4-(8-methoxyquinolin)-4-ylmethyl-6-(4- |
| ylmethyl)-3-(4-trifluoro- | trifluoromethanesulfonylphenyl)-4,6- |
| methanesulfonylphenyl)imidazolidine-2,4-dione | diazaspiro[2.4]heptane-5,7-dione |
| 5-methyl-1-(2-aminomethylpyrid-4-ylmethyl)-3-(4- | 4-(8-chloroquinolin)-4-ylmethyl-6-(4-trifluoromethane- |
| trifluoromethanesulfonylphenyl)imidazolidine-2,4- | sulfonylphenyl)-4,6-diazaspiro[2.4]heptane-5,7-dione |
| dione | |
| 5-methyl-1-(3-methylpyrid-4-ylmethyl)-3-(4- | 4-(8-fluoroquinolin)-4-ylmethyl-6-(4-trifluoromethane- |
| trifluoromethanesulfonylphenyl)imidazolidine-2,4- | sulfonylphenyl)-4,6-diazaspiro[2.4]heptane-5,7-dione |
| dione | |
| 5-methyl-1-(3-ethylpyrid-4-ylmethyl)-3-(4-trifluoro- | 4-(8-aminoquinolin)-4-ylmethyl-6-(4-trifluoromethane- |
| methanesulfonylphenyl)imidazolidine-2,4-dione | sulfonylphenyl)-4,6-diazaspiro[2.4]heptane-5,7-dione |
| 5-methyl-1-(3-isopropylpyrid-4-ylmethyl)-3-(4- | 4-(8-methylaminoquinolin)-4-ylmethyl-6-(4-trifluoro- |
| trifluoromethanesulfonylphenyl)imidazolidine-2,4- | methanesulfonylphenyl)-4,6-diazaspiro[2.4]heptane- |
| dione | 5,7-dione |
| 5-methyl-1-(3-tert-butylpyrid-4-ylmethyl)-3-(4- | 4-(8-dimethylaminoquinolin)-4-ylmethyl-6-(4-trifluoro- |
| trifluoromethanesulfonylphenyl)imidazolidine-2,4- | methanesulfonylphenyl)-4,6-diazaspiro[2.4]heptane- |
| dione | 5,7-dione |
| 5-methyl-1-(3-isobutylpyrid-4-ylmethyl)-3-(4- | 4-(8-piperidinoquinolin)-4-ylmethyl-6-(4-trifluoro- |
| trifluoromethanesulfonylphenyl)imidazolidine-2,4- | methanesulfonylphenyl)-4,6-diazaspiro[2.4]heptane- |
| dione | 5,7-dione |
| 5-methyl-1-(3-cyclohexylpyrid-4-ylmethyl)-3-(4- | 5-methyl-1-(2-methylquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-phenylpyrid-4-ylmethyl)-3-(4- | 5-methyl-1-(2-methoxyquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-[3-(4-chlorophenyl)pyrid-4-ylmethyl]-3- | 5-methyl-1-(2-chloroquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[3-(3-chlorophenyl)pyrid-4-ylmethyl]-3- | 5-methyl-1-(2-fluoroquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[3-(2-chlorophenyl)pyrid-4-ylmethyl]-3- | 5-methyl-1-(2-aminoquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[3-(4-fluorophenyl)pyrid-4-ylmethyl]-3- | 5-methyl-1-(2-methylaminoquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[3-(4-methoxyphenyl)pyrid-4-ylmethyl]- | 5-methyl-1-(2-dimethylaminoquinolin-4-ylmethyl)-3- |
| 3-(4-trifluoromethanesulfonylphenyl)imidazolidine- | (4-trifluoromethanesulfonylphenyl)imidazolidine-2,4- |
| 2,4-dione | dione |
| 5-methyl-1-(3-benzylpyrid-4-ylmethyl)-3-(4- | 5-methyl-1-(2-piperidinoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3,2′-bipyrid-4-ylmethyl)-3-(4-trifluoro- | 5-methyl-1-(3-methylquinolin-4-ylmethyl)-3-(4- |
| methanesulfonylphenyl)imidazolidine-2,4-dione | trifluoromethanesulfonylphenyl)imidazolidine-2,4- |
| | dione |
| 5-methyl-1-(3-hydroxymethylpyrid-4-ylmethyl)-3- | 5-methyl-1-(3-methoxyquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-(3-chloropyrid-4-ylmethyl)-3-(4- | 5-methyl-1-(3-chloroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-fluoropyrid-4-ylmethyl)-3-(4- | 5-methyl-1-(3-fluoroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-bromopyrid-4-ylmethyl)-3-(4- | 5-methyl-1-(3-aminoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-aminopyrid-4-ylmethyl)-3-(4- | 5-methyl-1-(3-methylaminoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-methylaminopyrid-4-ylmethyl)-3-(4- | 5-methyl-1-(3-dimethylaminoquinolin-4-ylmethyl)-3- |
| trifluoromethanesulfonylphenyl)imidazolidine-2,4- | (4-trifluoromethanesulfonylphenyl)imidazolidine-2,4- |
| dione | dione |
| 5-methyl-1-(3-ethylaminopyrid-4-ylmethyl)-3-(4- | 5-methyl-1-(3-piperidinoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-isopropylaminopyrid-4-ylmethyl)-3- | 5-methyl-1-(5-methylquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-(3-phenylaminopyrid-4-ylmethyl)-3-(4- | 5-methyl-1-(5-methoxyquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-cyclohexylaminopyrid-4-ylmethyl)-3- | 5-methyl-1-(5-chloroquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-(3-diethylaminopyrid-4-ylmethyl)-3-(4- | 5-methyl-1-(5-fluoroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-N-cyclohexyl-N-methylaminopyrid- | 5-methyl-1-(5-aminoquinolin-4-ylmethyl)-3-(4- |
| 4-ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(3-piperidinopyrid-4-ylmethyl)-3-(4- | 5-methyl-1-(5-methylaminoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-morpholinopyrid-4-ylmethyl)-3-(4- | 5-methyl-1-(5-dimethylaminoquinolin-4ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-[3-(4- | 5-methyl-1-(5-piperidinoquinolin-4-ylmethyl)-3-(4- |
| methylpiperazino)diethylaminopyrid-4-ylmethyl]-3- | trifluoromethanesulfonylphenyl)imidazolidine-2,4- |
| (4-trifluoromethanesulfonylphenyl)imidazolidine- | dione |
| 2,4-dione | |
| 5-methyl-1-[3-(1H-pyrazol-1-yl)pyrid-4-ylmethyl]- | 5-methyl-1-(6-methylquinolin-4-ylmethyl)-3-(4- |
| 3-(4-trifluoromethanesulfonylphenyl)imidazolidine- | trifluoromethanesulfonylphenyl)imidazolidine-2,4- |
| 2,4-dione | dione |
| 5-methyl-1-[3-(2-benzoylhydrazino)pyrid-4- | 5-methyl-1-(6-methoxyquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-benzoylaminopyrid-4-ylmethyl)-3-(4- | 5-methyl-1-(6-chloroquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-[3-(4-chlorobenzoylamino)pyrid-4- | 5-methyl-1-(6-fluoroquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[3-(3-chlorobenzoylamino)pyrid-4- | 5-methyl-1-(6-aminoquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[3-(2-chlorobenzoylamino)pyrid-4- | 5-methyl-1-(6-methylaminoquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[3-(4-fluorobenzoylamino)pyrid-4- | 5-methyl-1-(6-dimethylaminoquinolin-4-ylmethyl)-3- |
| ylmethyl]-3-(4-trifluoro- | (4-trifluoromethanesulfonylphenyl)imidazolidine-2,4- |
| methanesulfonylphenyl)imidazolidine-2,4-dione | dione |
| 5-methyl-1-[3-(4-methoxybenzoylamino)pyrid-4- | 5-methyl-1-(6-piperidinoquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-acetamidopyrid-4-ylmethyl)-3-(4- | 5-methyl-1-(7-methylquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-phenylacetamidopyrid-4-ylmethyl)-3- | 5-methyl-1-(7-methoxyquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[3-(4-methylpiperazino)acetamidopyrid- | 5-methyl-1-(7-chloroquinolin-4-ylmethyl)-3-(4- |
| 4-ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-cyclohexanylcarboxamidopyrid-4- | 5-methyl-1-(7-fluoroquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[3-(1-methyl-4- | 5-methyl-1-(7-aminoquinolin-4-ylmethyl)-3-(4- |
| piperidinyl)carbonylaminopyrid-4-ylmethyl]-3-(4- | trifluoromethanesulfonylphenyl)imidazolidine-2,4- |
| trifluoromethanesulfonylphenyl)imidazolidine-2,4- | dione |
| dione | |
| 5-methyl-1-[3-(2-furanylcarbonyl)aminopyrid-4- | 5-methyl-1-(7-methylaminoquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-ethoxycarbonylaminopyrid-4- | 5-methyl-1-(7-dimethylaminoquinolin-4-ylmethyl)-3- |
| ylmethyl)-3-(4-trifluoro- | (4-trifluoromethanesulfonylphenyl)imidazolidine-2,4- |
| methanesulfonylphenyl)imidazolidine-2,4-dione | dione |
| 5-methyl-1-(3-isopropoxycarbonylaminopyrid-4- | 5-methyl-1-(7-piperidinoquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(3-butoxycarbonylaminopyrid-4- | 5-methyl-1-(8-methylquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[3-(2- | 5-methyl-1-(8-methoxyquinolin-4-ylmethyl)-3-(4- |
| diethylaminoethoxycarbonylamino)pyrid-4- | trifluoromethanesulfonylphenyl)imidazolidine-2,4- |
| ylmethyl]-3-(4-trifluoro- | dione |
| methanesulfonylphenyl)imidazolidine-2,4-dione | |
| 5-methyl-1-(3-phenoxycarbonylaminopyrid-4- | 5-methyl-1-(8-chloroquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[3-(4-chloroanilino)carbonylaminopyrid- | 5-methyl-1-(8-fluoroquinolin-4-ylmethyl)-3-(4- |
| 4-ylmethyl)]-3-(4-trifluoro- | trifluoromethanesulfonylphenyl)imidazolidine-2,4- |
| methanesulfonylphenyl)imidazolidine-2,4-dione | dione |
| 5-methyl-1-(3-anilinocarbonylaminopyrid-4- | 5-methyl-1-(8-aminoquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(3-benzylaminocarbonylaminopyrid-4- | 5-methyl-1-(8-methylaminoquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[3-(2- | 5-methyl-1-(8-dimethylaminoquinolin-4-ylmethyl)-3- |
| pyridylamino)carbonylaminopyrid-4-ylmethyl)]-3- | (4-trifluoromethanesulfonylphenyl)imidazolidine-2,4- |
| (4-trifluoromethanesulfonylphenyl)imidazolidine- | dione |
| 2,4-dione | |
| 5-methyl-1-(3-ureidopyrid-4-ylmethyl)-3-(4- | 5-methyl-1-(8-piperidinoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-benzylaminosulfonylaminopyrid-4- | 1-(2-methylquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(3-methylaminosulfonylaminopyrid-4- | 1-(2-methoxyquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(3-aminosulfonylaminopyrid-4- | 1-(2-chloroquinolin-4-ylmethyl)-3-(4-trifluoromethane- |
| ylmethyl)-3-(4-trifluoro- | sulfonylphenyl)imidazolidine-2,4-dione |
| methanesulfonylphenyl)imidazolidine-2,4-dione | |
| 5-methyl-1-(3-morpholinosulfonylaminopyrid-4- | 1-(2-fluoroquinolin-4-ylmethyl)-3-(4-trifluoromethane- |
| ylmethyl)-3-(4-trifluoro- | sulfonylphenyl)imidazolidine-2,4-dione |
| methanesulfonylphenyl)imidazolidine-2,4-dione | |
| 5-methyl-1-(3-dimethylaminosulfonylaminopyrid-4- | 1-(2-aminoquinolin-4-ylmethyl)-3-(4-trifluoromethane- |
| ylmethyl)-3-(4-trifluoro- | sulfonylphenyl)imidazolidine-2,4-dione |
| methanesulfonylphenyl)imidazolidine-2,4-dione | |
| 5-methyl-1-(3-anilinosulfonylaminopyrid-4- | 1-(2-methylaminoquinolin-4-ylmethyl)-3-(4-trifluoro- |
| ylmethyl)-3-(4-trifluoro- | methanesulfonylphenyl)imidazolidine-2,4-dione |
| methanesulfonylphenyl)imidazolidine-2,4-dione | |
| 5-methyl-1-(3-phenylsulfonylaminopyrid-4- | 1-(2-dimethylaminoquinolin-4-ylmethyl)-3-(4-trifluoro- |
| ylmethyl)-3-(4-trifluoro- | methanesulfonylphenyl)imidazolidine-2,4-dione |
| methanesulfonylphenyl)imidazolidine-2,4-dione | |
| 5-methyl-1-[3-(2-thienyl)sulfonylaminopyrid-4- | 1-(2-piperidinoquinolin-4-ylmethyl)-3-(4-trifluoro- |
| ylmethyl]-3-(4-trifluoro- | methanesulfonylphenyl)imidazolidine-2,4-dione |
| methanesulfonylphenyl)imidazolidine-2,4-dione | |
| 5-methyl-1-(3-methylsulfonylaminopyrid-4- | 1-(3-methylquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[3-(4-chlorophenyl)sulfonylaminopyrid- | 1-(3-methoxyquinolin-4-ylmethyl)-3-(4- |
| 4-ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-benzylsulfonylaminopyrid-4- | 1-(3-chloroquinolin-4-ylmethyl)-3-(4-trifluoromethane- |
| ylmethyl)-3-(4-trifluoro- | sulfonylphenyl)imidazolidine-2,4-dione |
| methanesulfonylphenyl)imidazolidine-2,4-dione | |
| 5-methyl-1-(3-hydroxycarbonylpyrid-4-ylmethyl)-3- | 1-(3-fluoroquinolin-4-ylmethyl)-3-(4-trifluoromethane- |
| (4-trifluoromethanesulfonylphenyl)imidazolidine- | sulfonylphenyl)imidazolidine-2,4-dione |
| 2,4-dione | |
| 5-methyl-1-(3-ethoxycarbonylpyrid-4-ylmethyl)-3- | 1-(3-aminoquinolin-4-ylmethyl)-3-(4-trifluoromethane- |
| (4-trifluoromethanesulfonylphenyl)imidazolidine- | sulfonylphenyl)imidazolidine-2,4-dione |
| 2,4-dione | |
| 5-methyl-1-(3-tert-butoxycarbonylpyrid-4- | 1-(3-methylaminoquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-(3-phenoxycarbonylpyrid-4-ylmethyl)-3- | 1-(3-dimethylaminoquinolin-4-ylmethyl)-3-(4- |
| (4-trifluoromethanesulfonylphenyl)imidazolidine- | trifluoromethanesulfonylphenyl)imidazolidine-2,4- |
| 2,4-dione | dione |
| 5-methyl-1-[3-(4-methylpiperazinocarbonyl)pyrid-4- | 1-(3-piperidinoquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[3-(methylamino)carbonylpyrid-4- | 1-(5-methylquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[3-(dimethylamino)carbonylpyrid-4- | 1-(5-methoxyquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-piperidinocarbonylpyrid-4-ylmethyl)- | 1-(5-chloroquinolin-4-ylmethyl)-3-(4-trifluoromethane- |
| 3-(4-trifluoromethanesulfonylphenyl)imidazolidine- | sulfonylphenyl)imidazolidine-2,4-dione |
| 2,4-dione | |
| 5-methyl-1-(3-morpholinocarbonylpyrid-4- | 1-(5-fluoroquinolin-4-ylmethyl)-3-(4-trifluoromethane- |
| ylmethyl)-3-(4-trifluoro- | sulfonylphenyl)imidazolidine-2,4-dione |
| methanesulfonylphenyl)imidazolidine-2,4-dione | |
| 5-methyl-1-[3-(4-phenylpiperazinocarbonyl)pyrid-4- | 1-(5-aminoquinolin-4-ylmethyl)-3-(4-trifluoromethane- |
| ylmethyl]-3-(4-trifluoro- | sulfonylphenyl)imidazolidine-2,4-dione |
| methanesulfonylphenyl)imidazolidine-2,4-dione | |
| 5-methyl-1-(3-phenylaminocarbonylpyrid-4- | 1-(5-methylaminoquinolin-4-ylmethyl)-3-(4- |
| ylmethyl)-3-(4-trifluoro- | trifluoromethanesulfonylphenyl)imidazolidine-2,4- |
| methanesulfonylphenyl)imidazolidine-2,4-dione | dione |
| 5-methyl-1-[3-(methylphenylamino)carbonylpyrid- | 1-(5-dimethylaminoquinolin-4-ylmethyl)-3-(4- |
| 4-ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[3-(4- | 1-(5-piperidinoquinolin-4-ylmethyl)-3-(4- |
| methoxyphenyl)methylaminocarbonylpyrid-4- | trifluoromethanesulfonylphenyl)imidazolidine-2,4- |
| ylmethyl]-3-(4-trifluoro- | dione |
| methanesulfonylphenyl)imidazolidine-2,4-dione | |
| 5-methyl-1-[3-(4- | 1-(6-methylquinolin-4-ylmethyl)-3-(4- |
| chlorophenyl)methylaminocarbonylpyrid-4- | trifluoromethanesulfonylphenyl)imidazolidine-2,4- |
| ylmethyl]-3-(4-trifluoro- | dione |
| methanesulfonylphenyl)imidazolidine-2,4-dione | |
| 5-methyl-1-[3-(4-morpholinylamino)carbonylpyrid- | 1-(6-methoxyquinolin-4-ylmethyl)-3-(4- |
| 4-ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-aminocarbonylpyrid-4-ylmethyl)-3- | 1-(6-chloroquinolin-4-ylmethyl)-3-(4-trifluoromethane- |
| (4-trifluoromethanesulfonylphenyl)imidazolidine- | sulfonylphenyl)imidazolidine-2,4-dione |
| 2,4-dione | |
| 5-methyl-1-(3-phenoxypyrid-4-ylmethyl)-3-(4- | 1-(6-fluoroquinolin-4-ylmethyl)-3-(4-trifluoromethane- |
| trifluoromethanesulfonylphenyl)imidazolidine-2,4- | sulfonylphenyl)imidazolidine-2,4-dione |
| dione | |
| 5-methyl-1-(3-methoxypyrid-4-ylmethyl)-3-(4- | 1-(6-aminoquinolin-4-ylmethyl)-3-(4-trifluoromethane- |
| trifluoromethanesulfonylphenyl)imidazolidine-2,4- | sulfonylphenyl)imidazolidine-2,4-dione |
| dione | |
| 5-methyl-1-(3-ethoxypyrid-4-ylmethyl)-3-(4- | 1-(6-methylaminoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-[3-(1-pyrrolidinyl)ethoxypyrid-4- | 1-(6-dimethylaminoquinolin-4-ylmethyl)-3-(4- |
| ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-(3-benzyloxypyrid-4-ylmethyl)-3-(4- | 1-(6-piperidinoquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-(3-benzoyloxypyrid-4-ylmethyl)-3-(4- | 1-(7-methylquinolin-4-ylmethyl)-3-(4- |
| trifluoromethanesulfonylphenyl)imidazolidine-2,4- | trifluoromethanesulfonylphenyl)imidazolidine-2,4- |
| dione | dione |
| 5-methyl-1-[3-(4,5-dihydro-1H-imidazol-2-yl)pyrid- | 1-(7-methoxyquinolin-4-ylmethyl)-3-(4- |
| 4-ylmethyl]-3-(4-trifluoro- | trifluoromethanesulfonylphenyl)imidazolidine-2,4- |
| methanesulfonylphenyl)imidazolidine-2,4-dione | dione |
| 5-methyl-1-[3-(1H-imidazol-2-yl)pyrid-4-ylmethyl]- | 1-(7-chloroquinolin-4-ylmethyl)-3-(4-trifluoromethane- |
| 3-(4-trifluoromethanesulfonylphenyl)imidazolidine- | sulfonylphenyl)imidazolidine-2,4-dione |
| 2,4-dione | |
| 5-methyl-1-[3-(1H-tetrazol-5-yl)pyrid-4-ylmethyl]- | 1-(7-fluoroquinolin-4-ylmethyl)-3-(4-trifluoromethane- |
| 3-(4-trifluoromethanesulfonylphenyl)imidazolidine- | sulfonylphenyl)imidazolidine-2,4-dione |
| 2,4-dione | |
| 5-methyl-1-(3-amidinopyrid-4-ylmethyl)-3-(4- | 1-(7-aminoquinolin-4-ylmethyl)-3-(4-trifluoromethane- |
| trifluoromethanesulfonylphenyl)imidazolidine-2,4- | sulfonylphenyl)imidazolidine-2,4-dione |
| dione | |
| 5-methyl-1-(3-dimethylaminomethylpyrid-4- | 1-(7-methylaminoquinolin-4-ylmethyl)-3-(4-trifluoro- |
| ylmethyl)-3-(4-trifluoro- | methanesulfonylphenyl)imidazolidine-2,4-dione |
| methanesulfonylphenyl)imidazolidine-2,4-dione | |
| 5-methyl-1-(3-aminomethylpyrid-4-ylmethyl)-3-(4- | 1-(7-dimethylaminoquinolin-4-ylmethyl)-3-(4-trifluoro- |
| trifluoromethanesulfonylphenyl)imidazolidine-2,4- | methanesulfonylphenyl)imidazolidine-2,4-dione |
| dione | |
| 1-(2-methylpyrid-4-ylmethyl)-3-(4-trifluoro- | 1-(7-piperidinoquinolin-4-ylmethyl)-3-(4-trifluoro- |
| methanesulfonylphenyl)imidazolidine-2,4-dione | methanesulfonylphenyl)imidazolidine-2,4-dione |
| 1-(2-ethylpyrid-4-ylmethyl)-3-(4-trifluoro- | 1-(8-methylquinolin-4-ylmethyl)-3-(4- |
| methanesulfonylphenyl)imidazolidine-2,4-dione | trifluoromethanesulfonylphenyl)imidazolidine-2,4- |
| | dione |
| 1-(2-isopropylpyrid-4-ylmethyl)-3-(4-trifluoro- | 1-(8-methoxyquinolin-4-ylmethyl)-3-(4- |
| methanesulfonylphenyl)imidazolidine-2,4-dione | trifluoromethanesulfonylphenyl)imidazolidine-2,4- |
| | dione |
| 1-(2-tert-butylpyrid-4-ylmethyl)-3-(4-trifluoro- | 1-(8-chloroquinolin-4-ylmethyl)-3-(4-trifluoromethane- |
| methanesulfonylphenyl)imidazolidine-2,4-dione | sulfonylphenyl)imidazolidine-2,4-dione |
| 1-(2-isobutylpyrid-4-ylmethyl)-3-(4-trifluoro- | 1-(8-fluoroquinolin-4-ylmethyl)-3-(4-trifluoro- |
| methanesulfonylphenyl)imidazolidine-2,4-dione | methanesulfonylphenyl)imidazolidine-2,4-dione |
| 1-(2-cyclohexylpyrid-4-ylmethyl)-3-(4-trifluoro- | 1-(8-aminoquinolin-4-ylmethyl)-3-(4-trifluoromethane- |
| methanesulfonylphenyl)imidazolidine-2,4-dione | sulfonylphenyl)imidazolidine-2,4-dione |
| 1-(2-phenylpyrid-4-ylmethyl)-3-(4-trifluoro- | 1-(8-methylaminoquinolin-4-ylmethyl)-3-(4-trifluoro- |
| methanesulfonylphenyl)imidazolidine-2,4-dione | methanesulfonylphenyl)imidazolidine-2,4-dione |
| 1-[2-(4-chlorophenyl)pyrid-4-ylmethyl]-3-(4- | 1-(8-dimethylaminoquinolin-4-ylmethyl)-3-(4-trifluoro- |
| trifluoromethanesulfonylphenyl)imidazolidine-2,4- | methanesulfonylphenyl)imidazolidine-2,4-dione |
| dione | |
| 1-[2-(3-chlorophenyl)pyrid-4-ylmethyl]-3-(4- | 1-(8-piperidinoquinolin-4-ylmethyl)-3-(4-trifluoro- |
| trifluoromethanesulfonylphenyl)imidazolidine-2,4- | methanesulfonylphenyl)imidazolidine-2,4-dione |
| dione | |
| 1-[2-(2-chlorophenyl)pyrid-4-ylmethyl]-3-(4- | |
| trifluoromethanesulfonylphenyl)imidazolidine-2,4- | |
| dione | |

In vitro and in vivo effects of the cyclic urea compounds and methods of preparing the preferred cyclic urea compounds of the invention are described in the Examples.

### EXAMPLES

### Example 1: Synthesis of Cyclic Urea Compounds

The compound of Formula (I) according to the present invention may be prepared by application or adaptation of known methods and especially of the methods described in the literature such as, for example, those described by R. C. Larock in: Comprehensive Organic Transformations, VCH publishers, 1989.

In the reactions described below, it may be necessary to protect reactive functional groups such as, for example, hydroxyl, amino, imino, thio or carboxyl groups, when these groups are desired in the final product but when their participation is not desired in the reactions for synthesizing the compound of Formula (I). Conventional protecting groups may be used in accordance with the usual standard practices, for instance those described, for example, by T. W. Greene and P. G. M. Wuts in “Protective Groups in Organic Chemistry” John Wiley and Sons, 1991.

The products of Formula (II) used at the start of the invention may be obtained by the action of phosgene when X is an oxygen atom, or of thiophosgene when X is a sulfur atom, on the corresponding amine of Formula (A), i.e., the aminophenyl derivative bearing the substituents Y and Y^{1′} as defined above.

A product of this type is also described in French patent 2 329 276.

The products of Formula (III) or (III′) are known or may be prepared from the corresponding cyanohydrin according to the process described in the publication: J. Am. Chem. Soc. (1953), 75, 4841.

The products of Formula (III) may be obtained by the action of a product of formula Y²—B²-A²-Hal on 2-cyano-2-aminopropane under the conditions stated above for the action of Y²—B²-A²-Hal on the products of Formula (IV). An example of a preparation of this type is described in Jilek et al. Collect. Czech. Chem. Comm. 54(8) 2248 (1989).

The products of Formula (IV′) are described in French patent 2 329 276.

The starting materials of Formulae (V) and (VI), on which a process that is the subject of the invention is performed, to obtain the compound of Formula (I), are known and commercially available or may be prepared according to methods known to those skilled in the art.

The preparation of products of Formula (VI) is described especially in the following publications:
Zhur. Préklad. Khim. 28, 969-75 (1955) (CA 50, 4881a, 1956);
Tetrahedron 43, 1753 (1987);
J. Org. Chem. 52, 2407 (1987);
Zh. Org. Khim. 21, 2006 (1985);
J. Fluor. Chem. 17, 345 (1981)
   or in:
German patent DRP 637 318 (1935);
European patent EP 0 130 875;
Japanese patent JP 81 121 524.

The products of Formula (VI) that are hydantoin derivatives are widely used and cited in the literature, for instance in the following articles:
J. Pharm. Pharmacol., 67, Vol. 19(4), p. 209-16 (1967);
Khim. Farm. Zh., 67, Vol. 1 (5) p. 51-2;
German patent 2 217 914;
European patent 0 091 596;
J. Chem. Soc. Perkin. Trans. 1, p. 219-21 (1974).

The compound of Formula (I) of the present patent application as defined above, for which p is 0 and which thus constitute hydantoin derivatives, may be synthesized according to the process indicated above and especially according to the general scheme below which describes this synthesis on a solid support. The protocol that follows this scheme gives the operating conditions for such a synthesis of the compound of Formula (I) of the present patent application on a solid support.

The experimental section below more particularly gives an illustration of such a synthesis on a solid support according to the above protocol with the preparation of Example 1-1 to Example 1-56 of the present patent application.

Such a synthesis may be performed according to the general protocol below. Rink resin, protected with an Fmoc group, is deprotected with a 20% solution of piperidine in DMF. The resulting amine resin is coupled with an amino acid protected with an Fmoc group, in the presence of diisopropylaminecarbodiimide (DIC) and hydroxybenzotriazole (HOBt). The supported N-Fmoc amino acid is then deprotected with a 20% solution of piperidine in DMF. The free amine is reacted with an aldehyde dissolved in a 50/50 mixture of THF and triethyl orthoformate (TEOF) to give a Schiff's base, which is reduced with sodium cyanoborohydride. The resulting amine is coupled with an isocyanate or an isothiocyanate to give the corresponding urea or thiourea. When the isocyanate is not commercially available, it may be prepared from the corresponding amine by reaction with ⅓ equivalent of triphosgene in the presence of 2 equivalents of pyridine. The product is then cleaved with a 95% trifluoroacetic acid/water mixture. The urea thus released cyclizes to give the expected hydantoinine. In certain cases, the cleavage solution must be heated to 80° C. to obtain complete cyclization.

The compound of Formula (I) of the present patent application as defined above, for which p is 1 and which thus constitute dihydrouracil derivatives, may be synthesized according to the process indicated above and especially according to the general scheme below which describes this synthesis on a solid support. The protocol that follows this scheme gives the operating conditions for such a synthesis of the compound of Formula (I) of the present patent application on a solid support.

The experimental section below more particularly gives an illustration of such a synthesis on a solid support according to the above protocol with the preparation of Example 1-5 of the present patent application.

For the synthesis of the dihydrouracils on a solid support, the protocol that follows may be used.

Wang polystyrene resin (1.7 mmol/g) is used, for example, which resin is treated with a mixture of β-amino acid, 2,6-dichlorobenzoyl chloride and pyridine in DMF. After washing, the resin is treated with a 10% solution of piperidine in DMF. The resulting free amine is reacted with an aldehyde in a mixture of THF/trimethyl orthoformate (TMOF). The resulting Schiff's base is reduced with sodium cyanoborohydride in a mixture of methanol, THF and acetic acid. The secondary amine obtained is acylated with phosgene and the resulting carbamoyl chloride is treated with a primary amine to give the corresponding urea. Cyclization to the dihydrouracil and cleavage of the final product are performed by treating with a strong base such as diazabicycloundecene (DBU).

The compound of Formula (I) of the present patent application may thus be synthesized on a solid support as described above or in liquid phase according to the process indicated below: the experimental section of the present patent application gives an illustration of such a liquid-phase synthesis with the preparation of Example 1-57 to Example 1-62. For this liquid-phase synthetic process, two routes A and B may be performed, each involving two steps.

Route A:

Step A: The alkylation of the amino ester may be performed by reductive amination with an aromatic or heterocyclic aldehyde according to the general process described in Advanced Organic Reaction, March, third edition, page 798-800. In particular, the formation of the Schiff s base (intermediate) may be performed using an amino ester optionally in salt form, an aldehyde and optionally a dehydrating agent (for example magnesium sulfate) in a solvent, for instance dichloromethane or dichloroethane, at a temperature of between 0° C. and the reflux point of the solvent. The imine formed may be isolated. The imine formed is reduced with a metal hydride, for instance sodium borohydride, in a solvent, for instance an alcohol (for example ethanol or methanol), at a temperature of between 0° C. and the reflux point of the solvent.
Step B: The amino ester obtained is coupled with an isocyanate in a solvent, for instance THF or dichloromethane, with or without the presence of a base (for example triethylamine) or an acid (for example trifluoroacetic acid), at a temperature of between 0° C. and the reflux point of the solvent. When the isocyanates are not commercially available, they are prepared from the corresponding amines and triphosgene or diphosgene or phosgene in the presence of a base (for example pyridine or triethylamine) according to the general procedure described in Advanced Organic Reaction, March, third edition, page 370.

Route B:

Step A: The formation of the isocyanate may be performed by coupling an aromatic or heterocyclic amine with diphosgene in the presence of activated plant charcoal, in a solvent, for instance toluene, at a temperature of between −40° C. and the reflux point of the solvent. The isocyanate formed is not isolated, and may react with the amino ester or its salt in the same solvent in the presence of a base, for instance triethylamine, at a temperature of between 0° C. and the reflux point of the solvent, to give the 3-arylimidazolidine-2,4-dione derivative.
Step B: The coupling of this derivative with an alkyl halide is performed in the presence of a base, for instance potassium tert-butoxide or sodium hydride, in a solvent, for instance THF or DMF, at a temperature of between 0° C. and the reflux point of the solvent.

The compound of Formula (I) of the present patent application which constitute Example 1-201 to Example 1-207 of the present patent application were prepared as indicated below in the experimental section and as indicated in the following schemes.

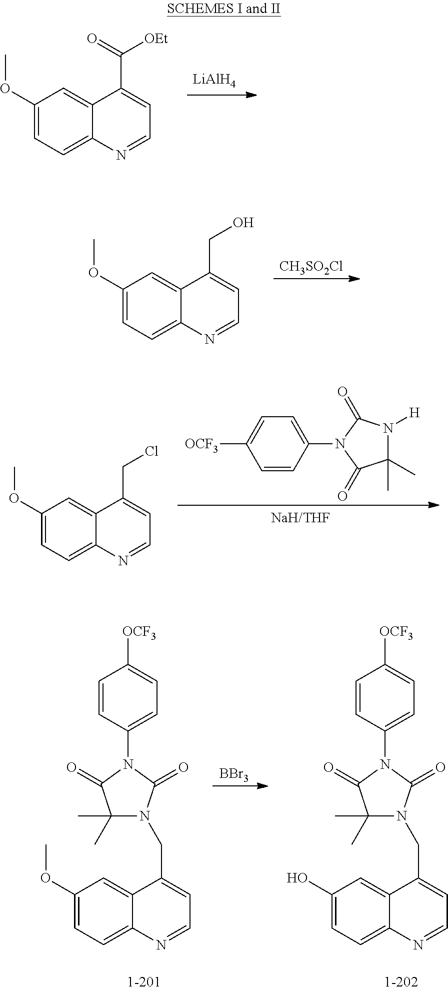

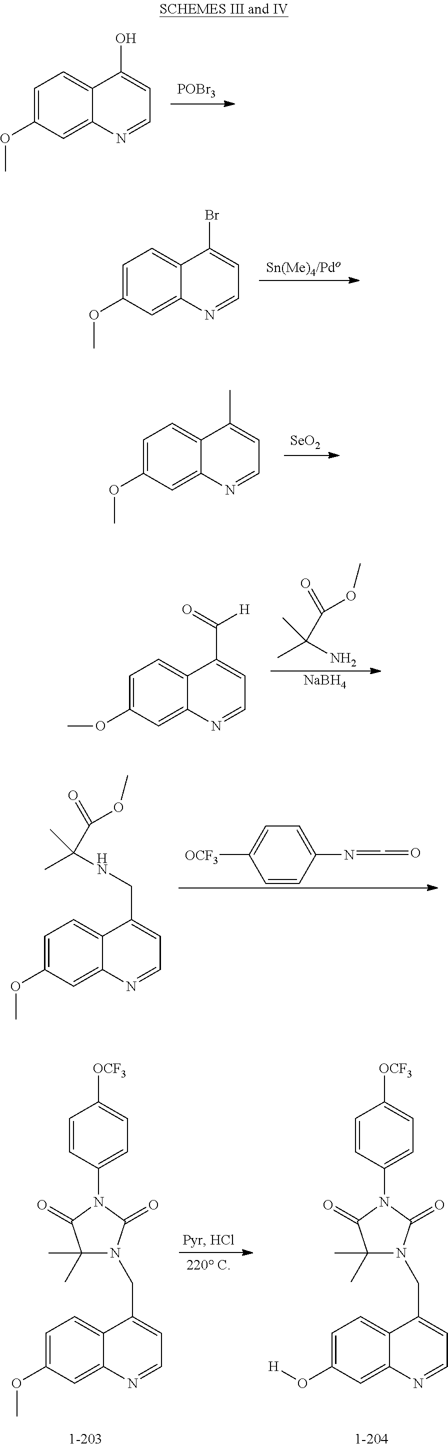

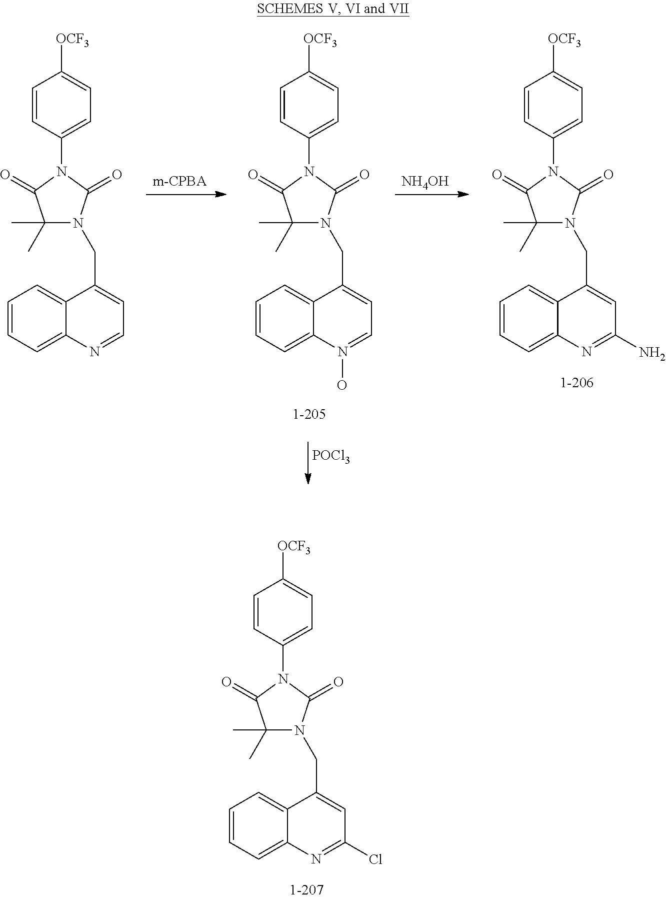

The synthesis of the compound of Formula (I) of the present patent application which constitute the products of Example 1-208 to Example 1-243 were performed using route B. The alkyl halide may be prepared from the corresponding carboxylic acids.

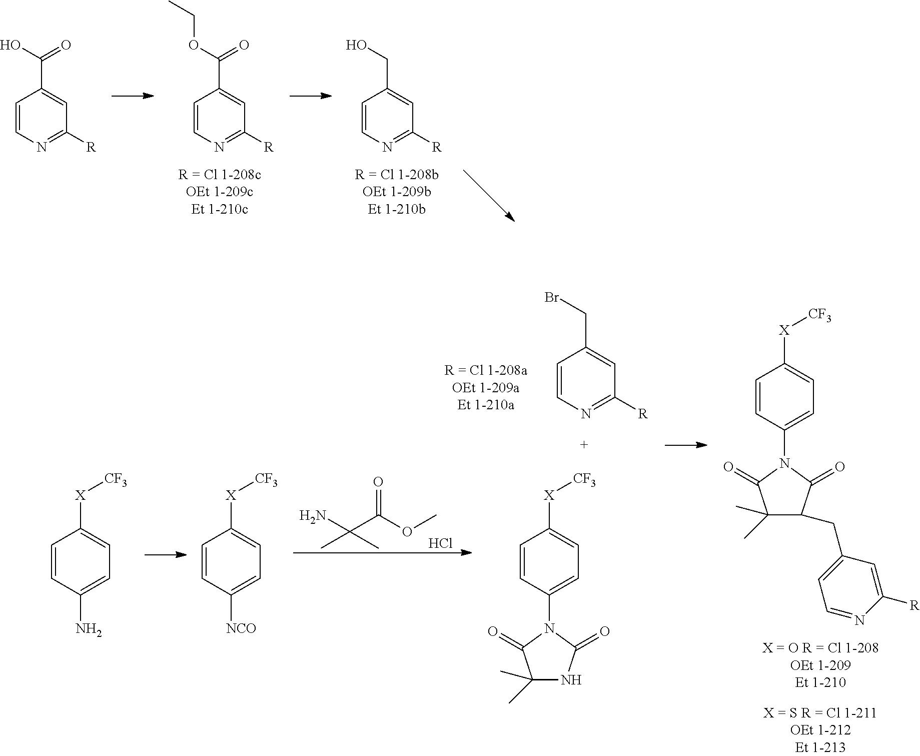

The ethyl carboxylates were prepared by esterification of the carboxylic acids in ethanol in the presence of sulfuric acid, adopting the conditions described in Synthesis 2000, 1138.

The reduction of the ethyl carboxylates in alcohol was performed in ethanol in the presence of sodium borohydride, adopting the conditions described in Synthesis 2000, 1665.

The conversion of the alcohols thus obtained into alkyl halides was performed using dibromotriphenylphosphorane as halogenating agent, adopting the conditions described in J. Heterocyclic Chem., 30, 631 (1993).

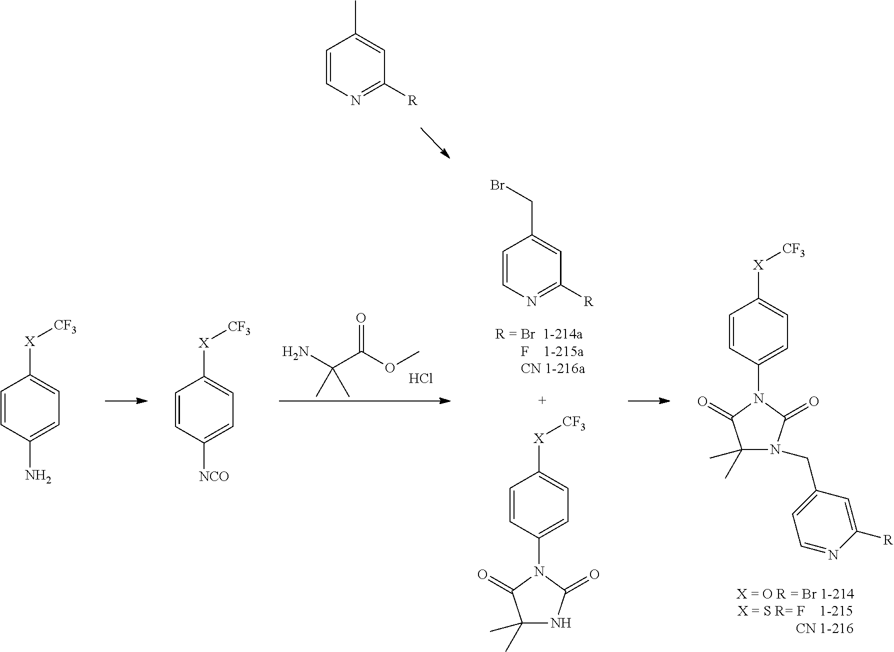

The alkyl halides may also be prepared by free-radical bromination of the corresponding methylenes in the presence of N-bromosuccinimide and benzoyl peroxide in carbon tetrachloride, adopting the conditions described in J. Heterocyclic Chem., 30, 631 (1993).

Thus, we functionalized the pyridine nucleus in position −2 with a bromine or fluorine atom or with a nitrile group.

The latter compound will also allow us to prepare various carbonyl compounds.

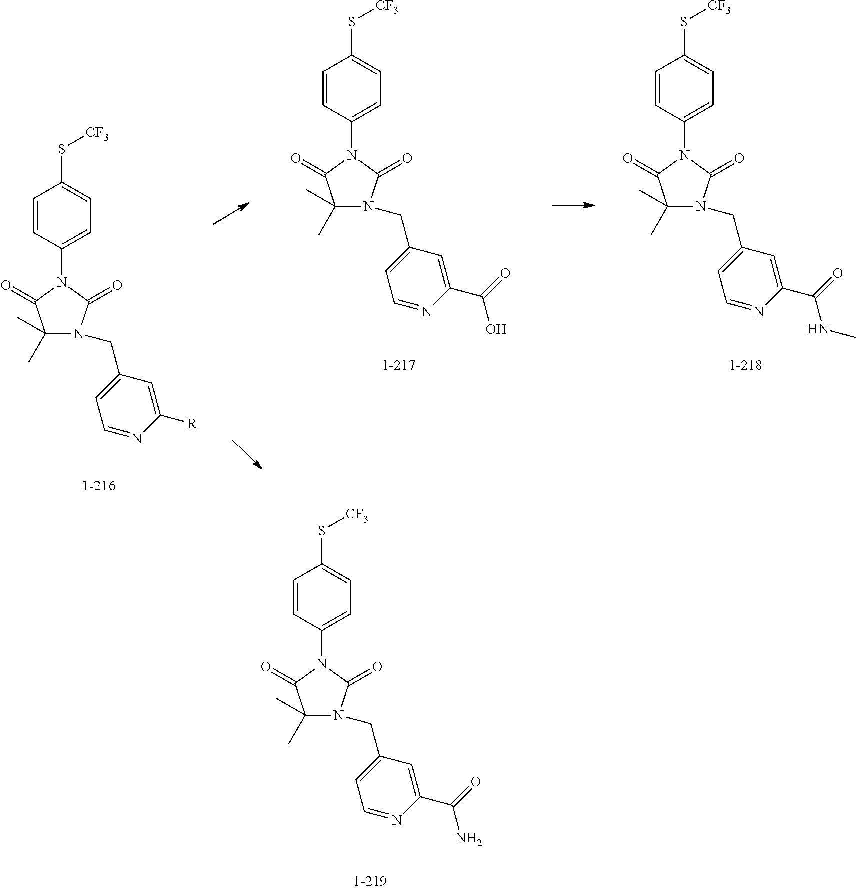

Acidic hydrolysis of the nitrile in the presence of sulfuric acid, adopting the conditions described in J. Med. Chem. 1991, 34, 281-290, led to the carboxamide in dimeric form. Hydrolysis of the nitrile under milder conditions allowed the expected carboxamide to be obtained.

Acidic hydrolysis of the nitrile in the presence of 5N hydrochloric acid, adopting the conditions described in J. Heterocyclic Chem., 30, 631 (1993), gave the corresponding carboxylic acid.

The amide could be obtained from the carboxylic acid using 1-(3-dimethylaminopropyl)-3-ethylcarbodiimide hydrochloride as coupling agent in dichloromethane, adopting the conditions described in J. Am. Chem. Soc., 95, 875, (1973).

Various amino analogs were obtained by nucleophilic substitution of the chloro intermediate with amines under irradiation in a microwave oven, inspired by the conditions described in Tetrahedron 2002, 58, 1125.

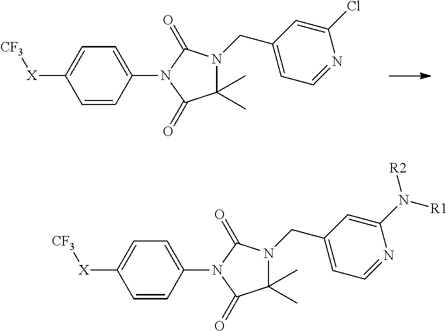

| | | Example | | Example | | Example |
|---|---|---|---|---|---|---|
| X | NR¹R² | No. | NR¹R² | No. | NR¹R² | No. |
| O | 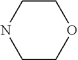 | 1-220 | | | | |
| S | 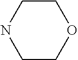 | 1-221 | 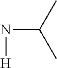 | 1-225 | 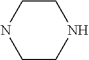 | 1-229 |
| | 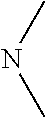 | 1-222 | 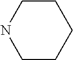 | 1-226 | 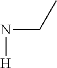 | 1-230 |
| | 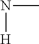 | 1-223 | 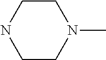 | 1-227 | 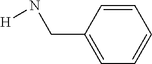 | 1-231 |
| | 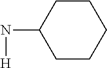 | 1-224 | 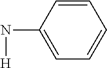 | 1-228 | 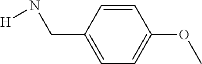 | 1-232 |

The amino analog was obtained by deprotection of the p-methoxybenzylamine group in the presence of trifluoroacetic acid, inspired by the conditions described in J. Chem. Soc., Perkin Trans. 1, 2002, 428-433.

This amino derivative allowed access to other chemical functions, for instance the amide function, the carbamate function or the sulfonamide function.

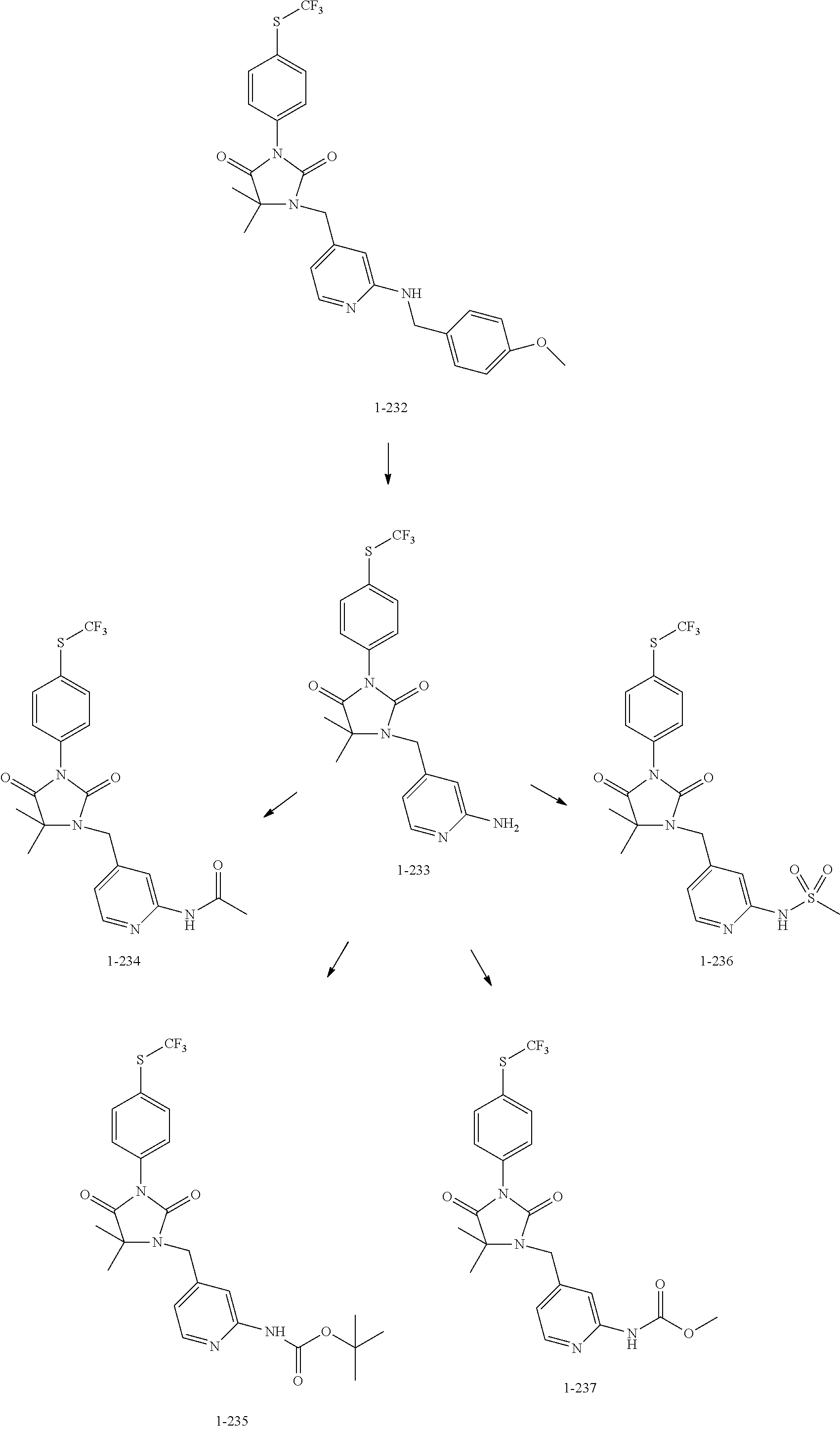 [SMILES: COc1ccc(CNc2cc(CN3C(=O)N(c4ccc(SC(F)(F)F)cc4)C(=O)C3(C)C)ccn2)cc1 ; CC(=O)Nc1cc(CN2C(=O)N(c3ccc(SC(F)(F)F)cc3)C(=O)C2(C)C)ccn1 ; CC1(C)C(=O)N(c2ccc(SC(F)(F)F)cc2)C(=O)N1Cc1ccnc(N)c1 ; CC1(C)C(=O)N(c2ccc(SC(F)(F)F)cc2)C(=O)N1Cc1ccnc(NS(C)(=O)=O)c1 ; CC(C)(C)OC(=O)Nc1cc(CN2C(=O)N(c3ccc(SC(F)(F)F)cc3)C(=O)C2(C)C)ccn1 ; COC(=O)Nc1cc(CN2C(=O)N(c3ccc(SC(F)(F)F)cc3)C(=O)C2(C)C)ccn1]

The acetamido derivative was obtained by acylation of the amino derivative in the presence of acetic anhydride, inspired by the conditions described in Tetrahedron Lett. 2002, 43, 3121. We also prepared the methyl and tert-butyl carbamates inspired by the conditions described in J. Heterocyclic Chem., 22, 313 (1985) and in J. Org. Chem. 2002, 67, 4965. The amino derivative was also sulfonylated with mesyl chloride, inspired by the conditions described in J. Med. Chem., 1985, 28, 824.

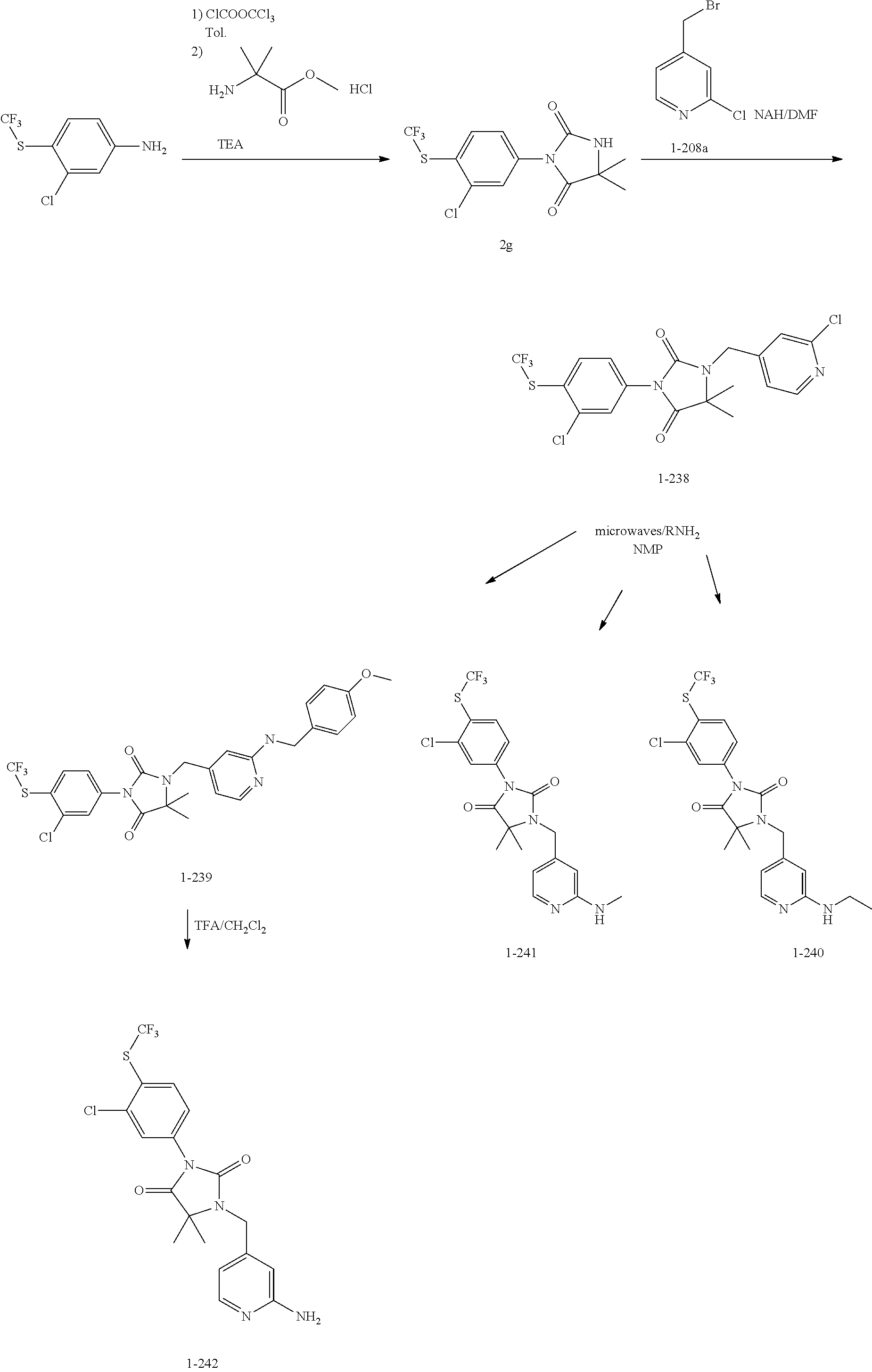

The above conditions were also used for the synthesis of the compounds containing a disubstituted phenyl group.

The hydantoin containing a pyridine nucleus disubstituted in position −2.6 with a bromine atom was prepared using the above conditions, starting with 2,6-dibromo-4-(hydroxymethyl)pyridine described in Synthesis 2000, 1665.

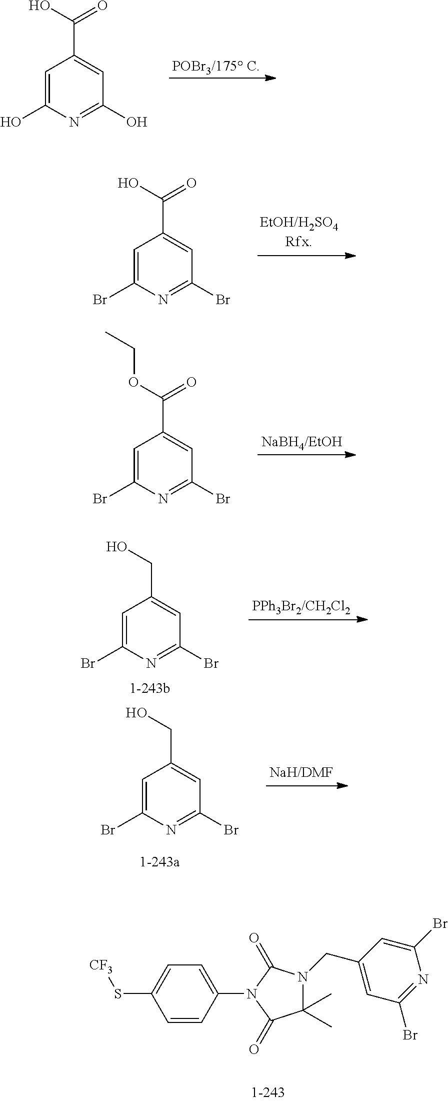

The products may be purified as follows:

### Purification by LC/MS

The products may be purified by LC/MS using a Waters FractionLynx system composed of a Waters model 600 gradient pump, a Waters model 515 regeneration pump, a Waters Reagent Manager dilution pump, a Waters model 2700 autoinjector, two Rheodyne LabPro model valves, a Waters model 996 diode array detector, a Waters model ZMD mass spectrometer and a Gilson model 204 fraction collector. The system was controlled by the Waters FractionLynx software. The separation was performed alternately on two Waters Symmetry columns (C₁₈, 5 μM, 19×50 mm, catalog reference 186000210), one column undergoing regeneration with a 95/5 (v/v) water/acetonitrile mixture containing 0.07% (v/v) trifluoroacetic acid, while the other column was performing separation. The columns were eluted using a linear gradient of from 5% to 95% of acetonitrile containing 0.07% (v/v) of trifluoroacetic acid in water containing 0.07% (v/v) trifluoroacetic acid, at a flow rate of 10 ml/minute. At the separation column outlet, one-thousandth of the effluent is separated by an LC Packing Accurate, diluted with methyl alcohol, at a flow rate of 0.5 ml/minute, and sent to the detectors, in a proportion of 75% to the diode array detector and the remaining 25% to the mass spectrometer. The rest of the effluent (999/1000) is sent to the fraction collector, where the flow is discarded as long as the mass of the expected product has not been detected by the FractionLynx software. The molecular formulae of the expected products are supplied to the FractionLynx software, which initiates the collection of the product when the mass signal detected corresponds to the ion [M+H]⁺ and/or to [M+Na]⁺. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, collection is also initiated when the mass signal of the ion [M+2H]⁺⁺ and/or [M+Na+H]⁺⁺ is detected. The products were collected in tared glass tubes. After collection, the solvents were evaporated off, in a Savant AES 2000 or Genevac HT8 centrifuge evaporator and the product masses were determined by weighing the tubes after evaporating off the solvents.

The LC/MS analyses were performed on a Micromass LCT model machine connected to an HP 1100 machine. The abundance of the products was measured using an HP G1315A diode array detector over a wavelength range from 200-600 nm and a Sedex 65 light scattering detector. The acquisition of the mass spectra was performed over a range from 180 to 800. The data were analyzed using the Micromass MassLynx software. The separation was performed on a Hypersil BDS C18, 3 μm column (50×4.6 mm), eluting with a linear gradient of from 5% to 90% of acetonitrile containing 0.05% (v/v) trifluoroacetic acid (TFA) in water containing 0.05% (v/v) TFA over 3.5 minutes at a flow rate of 1 ml/minute. The total analysis time, including the column reequilibration time, is 7 minutes.

The products of Example 1-244 to Example 1-255 of the present invention were prepared as indicated in the experimental section and according to the general synthetic route of the scheme below:

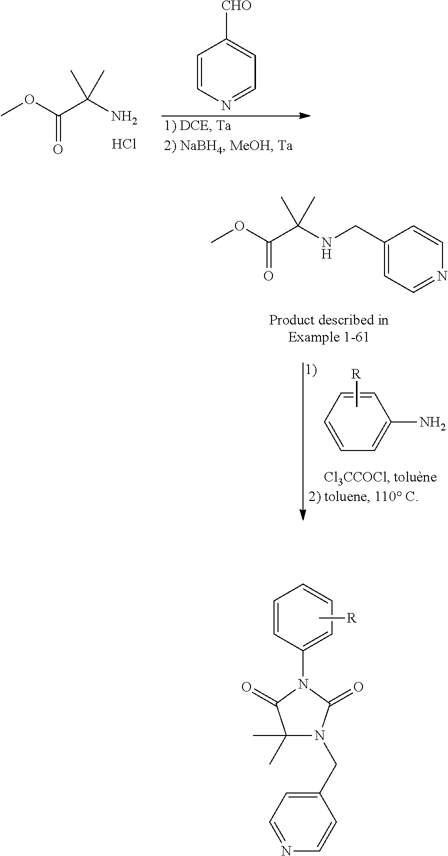

The products of Example 1-268 to Example 1-275 of the present invention were prepared according to reaction Schemes 1 and 2 indicated below, in which the FIGS. 1 to 8 correspond, respectively, to Example 1-268 to Example 1-275: the products of Example 1-268 to Example 1-273 (i.e. products 1 to 6) were prepared according to Scheme 1 and the two thiohydantoin compounds of Example 1-274 and Example 1-275 (i.e. products 7 and 8) were prepared according to Scheme 2.

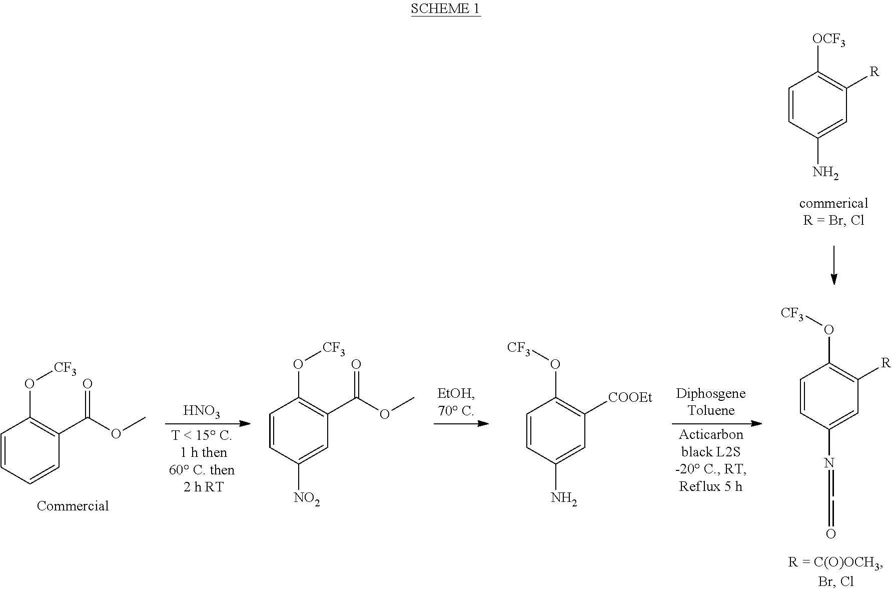

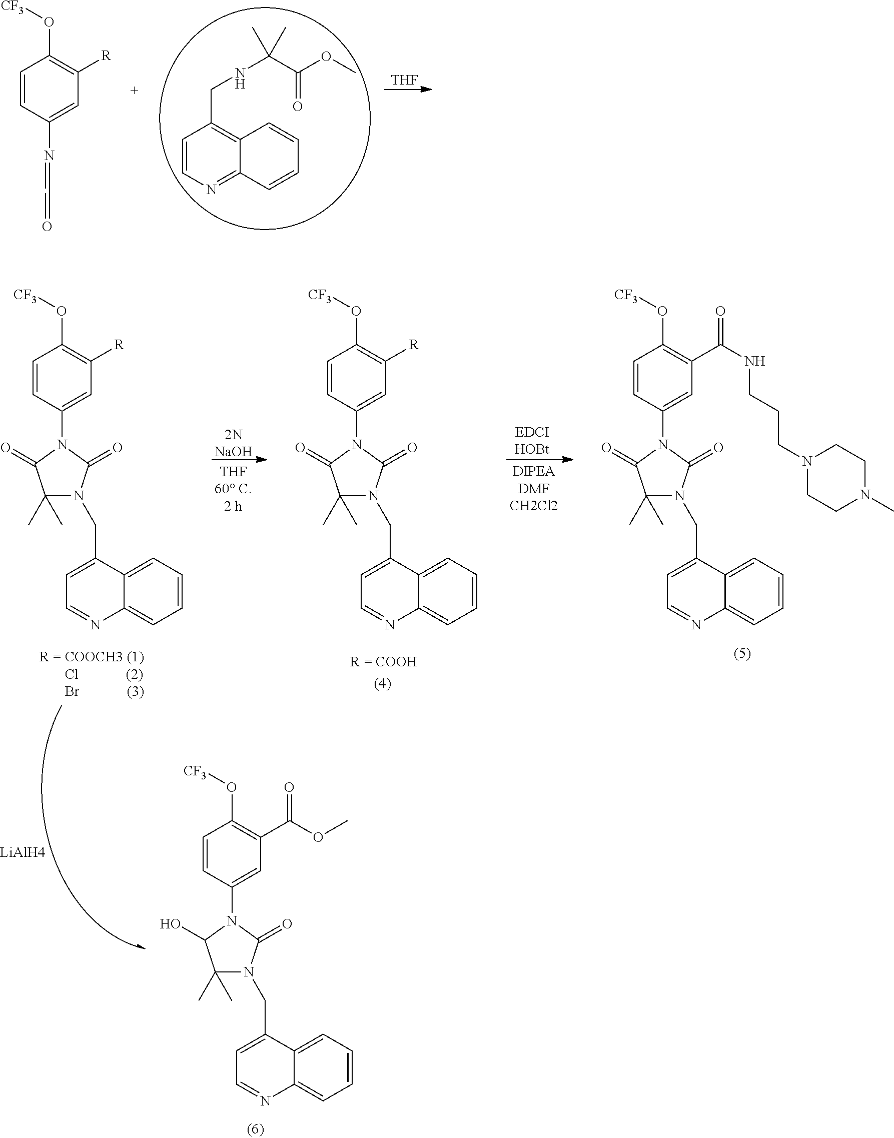

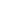

The nitro compound is prepared by nitration of methyl 2-trifluoromethoxybenzoate by nitration (fuming nitric acid) by controlling the temperature according to the conditions described in patent PCT Int. Appl. (2000), 564: WO 0069810.

The corresponding amine is prepared by reduction of the nitro function in the presence of SnCl₂ in ethanol, according to the same patent.

The isocyanate is prepared by reacting diphosgene dissolved in toluene at −20° C. under the usual known conditions. The isocyanate is reacted with the quinoline derivative prepared according to the known methods, in order to prepare the desired hydantoin.

The acid is obtained by saponification using 2N sodium hydroxide in THF at 60° C.

The amide is prepared by coupling the desired amine using EDCI as coupling agent (standard coupling conditions).

The alcohol is obtained by reducing the ester in THF in the presence of LiAlH₄.

The halo derivatives R═Cl and R═Br are prepared from the commercial anilines according to the same synthetic scheme.

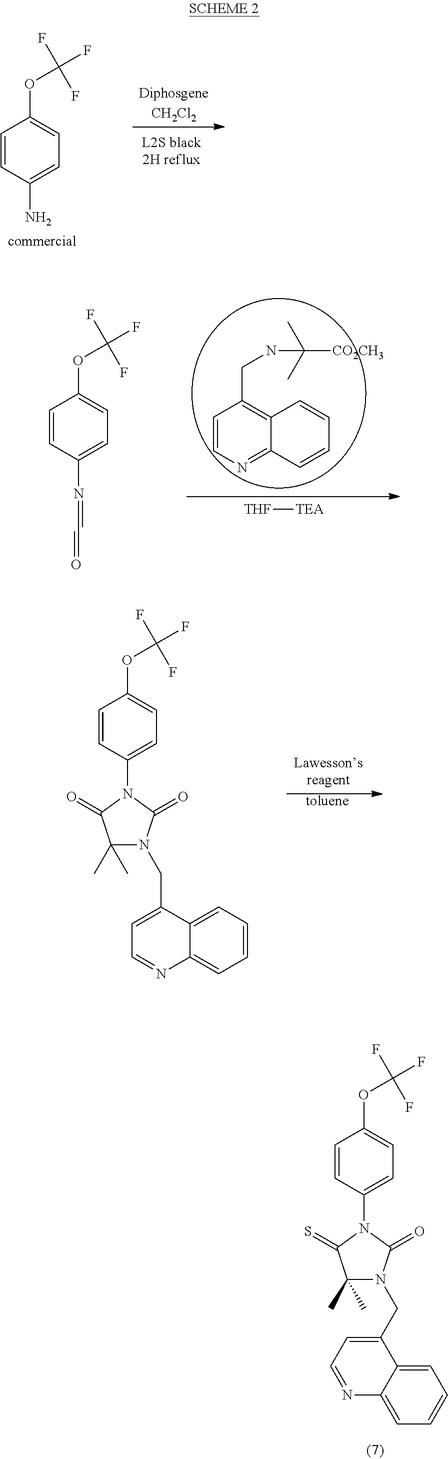

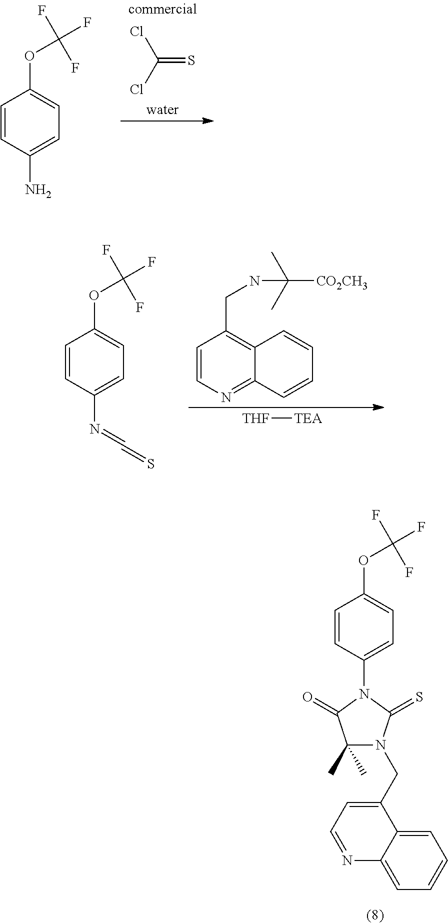

The examples whose preparation follows illustrate the present invention without, however, limiting it.

### Example 1-1: (S)-5-methyl-1-quinolin-4-ylmethyl-3-(4-trifluoromethanesulfonylphenyl)imidazolidine-2,4-dione trifluoroacetate

2 g (1.02 mmol) of Polystyrene AM RAM (Rink resin) (0.51 mmol/g) are suspended in 20 ml of DMF in a 50 ml syringe fitted with a sinter. After agitation for 10 minutes, the DMF is filtered and replaced with 10 ml of a 20% solution of piperidine in DMF. After agitation for one hour at room temperature, the solution is filtered and the resin washed successively with 3×10 ml of DMF, 2×10 ml of methanol and 3×10 ml of DMF. A solution of 0.94 g of Fmoc-Ala(OH) (3 mmol), 0.41 g of HOBt (3 mmol) and 0.48 ml of DIC (3 mmol) in 10 ml of DMF is added to the resin. The syringe is agitated overnight at room temperature and the resin is then washed successively with 5×10 ml of DMF, 3×10 ml of MeOH and 5×10 ml of DCM. Next, 10 ml of a 20% solution of piperidine in DMF are introduced into the syringe. After agitation for 1 hour, the solution is filtered and the resin washed with 5×10 ml of DMF, 2×10 ml of MeOH, 3×10 ml of DCM and 3×10 ml of THF. Next, a solution of 0.79 g of quinoline-4-carboxaldehyde (5.1 mmol) in 10 ml of a 50/50 THF/TEOF mixture is added to the resin. After agitation overnight at room temperature, the solution is filtered and the resin washed with 10×10 ml of THF. 0.63 g of sodium cyanoborohydride in a mixture of 1.5 ml of MeOH, 3.5 ml of dichloroethane and 0.1 ml of acetic acid is then added to the resin. The resin is agitated overnight and then, after filtration, washed with 10×10 ml of DCM, 3×10 ml of MeOH and 5×10 ml of DCM.

In parallel, a solution of 0.563 g of 4-(trifluoromethanesulfonyl)aniline (2.5 mmol) is treated with 0.25 g of triphosgene (0.83 mmol), followed by 0.23 ml of pyridine (2.5 mmol) at 0° C. under nitrogen. After the temperature has warmed gradually to room temperature, the reaction is stirred for 2 hours and a further 0.23 ml of pyridine in 1 ml of DMF is added to the mixture. The solution obtained is transferred into the syringe, which is agitated for 2 hours. The solution is then filtered and the resin washed with 5×10 ml of DCM, 3×10 ml of MeOH and 5×10 ml of DCM. Finally, the resin is treated with 5 ml of a 95% solution of trifluoroacetic acid in water. The mixture is agitated for 2 hours and then filtered. The resin is washed with 2 ml of MeOH, followed by 2 ml of DCM. The combined filtrates are evaporated under vacuum. 280 mg of crude product are thus obtained. After purification by preparative LC-MS, 240 mg (overall yield=41%) of expected product are isolated in the form of a white solid.

EIMS ([M+H]+: 464

Retention time (RT)=3.12 min (YMC basic S5 column; 2-85% ACN/H2O gradient over 7 min)

1H NMR (300 MHz) (CDCl3): 1.59 (d, 3H); 4.11 (t, 1H); 5.01 and 5.55 (AB, 2H); 7.70 (d, 1H); 7.89 (m, 1H); 7.99 (m, 3H); 8.15 (m, 2H); 8.30 (d, 1H), 8.50 (d, 1H); 9.22 (d, 1H).

### Example 1-2: (S)-4-methyl-3-quinol-4-ylmethyl-5-thioxo-1-(4-trifluoro-methanesulfonylphenyl)imidazolidin-2-one trifluoroacetate

Resin 3, 0.036 mmol, prepared according to Example 1-1, is used for the preparation of the compound.

33 mg of thiocarbonyldiimidazole (0.18 mmol) are added to a solution of 41 mg of 4-(trifluoromethanesulfonyl)aniline (0.18 mmol) in 3 ml of DCM. The reaction mixture is stirred for 2 hours at room temperature and then added directly to the resin. After stirring for 2 hours, the solution is filtered and the resin is then washed with 5×2 ml of DCM, 3×5 ml of MeOH and 5×2 ml of DCM.

Finally, 2 ml of a 95% solution of TFA in water are added to the resin. After stirring for 2 hours, the mixture is filtered and the resin washed with 1 ml of MeOH and 1 ml of DCM. The combined filtrates are heated at 60° C. for 2 hours and then concentrated under vacuum. After purification by preparative LC-MS, 1.8 mg of expected product are isolated.

EIMS [(M+H]+): 480

RT=4.72 min (YMC basic S5 column; 2-85% ACN/H2O gradient over 7 min)

### Example 1-3: (S)-5-methyl-1-pyrid-4-ylmethyl-3-(4-trifluoromethanesulfonylphenyl)imidazolidine-2,4-dione trifluoroacetate

The compound is prepared from 0.025 mmol of resin, 0.075 mmol of N-Fmoc-L-Ala(OH), 0.125 mmol of 4-pyridinecarboxaldehyde and 0.0625 mmol of 4-(trifluoro-methanesulfonyl)aniline, in the same way as in Example 1-1. After purification by preparative LC-MS, 5.6 mg of expected product are obtained.

EIMS ([M+H]+): 414

RT=2.72 min

### Example 1-4: (S)-5-methyl-1-pyrid-4-ylmethyl-3-(4-trifluoromethylsulfanylphenyl)imidazolidine-2,4-dione trifluoroacetate

The compound is prepared from 0.025 mmol of resin, 0.075 mmol of N-Fmoc-L-Ala(OH), 0.125 mmol of 4-pyridinecarboxaldehyde and 0.0625 mmol of 4-(trifluoro-methanethio)aniline, in the same way as in Example 1-1. After purification by preparative LC-MS, 1.3 mg of expected product are obtained.

EIMS ([M+H]+): 382

RT=2.83 min

### Example 1-5: (S)-5-methyl-1-quinol-4-ylmethyl-3-(4-trifluoromethylsulfanylphenyl)imidazolidine-2,4-dione trifluoroacetate

The compound is prepared from 0.025 mmol of resin, 0.075 mmol of N-Fmoc-L-Ala(OH), 0.125 mmol of 4-quinolinecarboxaldehyde and 0.0625 mmol of 4-(trifluoro-methanethio)aniline, in the same way as in Example 1-1. After purification by preparative LC-MS, 0.6 mg of expected product is obtained.

EIMS ([M+H]+): 432

RT=3.14 min

### Example 1-6: 1-quinol-4-ylmethyl-3-(4-trifluoromethanesulfonylphenyl)imidazolidine-2,4-dione trifluoroacetate

The compound is prepared from 4 mmol of resin, 12 mmol of N-Fmoc-Gly(OH), 20 mmol of 4-quinolinecarboxaldehyde, and 10 mmol of 4-(trifluoromethanesulfonyl)aniline, in the same way as in Example 1-1. After purification by preparative LC-MS, 1 g of expected product is obtained.

EIMS ([M+H]+): 450

RT=3.20 min

### Example 1-7: 5,5-dimethyl-1-quinol-4-ylmethyl-3-(4-trifluoromethanesulfonylphenyl)imidazolidine-2,4-dione trifluoroacetate

The compound is prepared from 0.25 mmol of resin, 0.75 mmol of Fmoc-AIB-(OH), 1.25 mmol of 4-quinolinecarboxaldehyde and 0.625 mmol of 4-(trifluoromethane-sulfonyl)aniline, in the same way as in Example 1-1. After purification by preparative LC-MS, 22 mg of expected product are obtained.

EIMS ([M+H]+): 478

RT=4.26 min

### Example 1-8: (R)-5-methyl-1-quinol-4-ylmethyl-3-(4-trifluoromethanesulfonylphenyl)imidazolidine-2,4-dione trifluoroacetate

The compound is prepared from 0.04 mmol of resin, 0.12 mmol of N-Fmoc-D-Ala(OH), 0.20 mmol of 4-quinolinecarboxaldehyde and 0.10 mmol of 4-(trifluoromethanesulfonyl)aniline, in the same way as in Example 1-1. After purification by preparative LC-MS, 10 mg of expected product are obtained.

EIMS ([M+H]+): 464

RT=4.36 min

### Example 1-9: (R)-5-methyl-1-quinol-4-ylmethyl-3-(4-trifluoromethylsulfanylphenyl)imidazolidine-2,4-dione trifluoroacetate

The compound is prepared from 0.04 mmol of resin, 0.12 mmol of N-Fmoc-D-Ala(OH), 0.20 mmol of 4-quinolinecarboxaldehyde and 0.10 mmol of 4-(trifluoromethanethio)aniline, in the same way as in Example 1-1. After purification by preparative LC-MS, 11 mg of expected product are obtained.

EIMS ([M+H]+): 432

RT=4.50 min

### Example 1-10: (R)-5-methyl-1-pyrid-4-ylmethyl-3-(4-trifluoromethanesulfonylphenyl)imidazolidine-2,4-dione trifluoroacetate

The compound is prepared from 0.28 mmol of resin, 0.84 mmol of N-Fmoc-D-Ala(OH), 1.4 mmol of 4-pyridinecarboxaldehyde and 0.70 mmol of 4-(trifluoromethane-sulfonyl)aniline, in the same way as in Example 1-1. After purification by preparative LC-MS, 105 mg of expected product are obtained.

EIMS ([M+H]+): 414

RT=2.40 min

### Example 1-11: (R)-5-methyl-1-pyrid-4-ylmethyl-3-(4-trifluoromethylsulfanylphenyl)imidazolidine-2,4-dione trifluoroacetate

The compound is prepared from 0.28 mmol of resin, 0.84 mmol of N-Fmoc-D-Ala(OH), 1.4 mmol of 4-pyridinecarboxaldehyde and 0.70 mmol of 4-(trifluoromethanethio)aniline, in the same way as in Example 1-1. After purification by preparative LC-MS, 91 mg of expected product are obtained.

EIMS ([M+H]+): 382

RT=2.52 min

### Example 1-12: (S)-5-methyl-1-(3-methylpyrid-4-ylmethyl)-3-(4-trifluoromethylsulfanylphenyl)imidazolidine-2,4-dione trifluoroacetate

The compound is prepared from 0.04 mmol of resin, 0.12 mmol of N-Fmoc-L-Ala(OH), 0.20 mmol of 3-methyl-4-pyridinecarboxaldehyde and 0.10 mmol of 4-(trifluoromethanethio)aniline, in the same way as in Example 1-1. After purification by preparative LC-MS, 17 mg of expected product are obtained.

EIMS ([M+H]+): 396

RT=4.20 min

### Example 1-13: (S)-5-methyl-1-(3-methylpyrid-4-ylmethyl)-3-(4-trifluoromethanesulfonylphenyl)imidazolidine-2,4-dione trifluoroacetate

The compound is prepared from 0.04 mmol of resin, 0.12 mmol of N-Fmoc-L-Ala(OH), 0.20 mmol of 3-methyl-4-pyridinecarboxaldehyde, and 0.10 mmol of 4-(trifluoromethanesulfonyl)aniline, in the same way as in Example 1-1. After purification by preparative LC-MS, 16 mg of expected product are obtained.

EIMS ([M+H]+): 428

RT=4.07 min

### Example 1-14: (S)-4-methyl-3-pyrid-4-ylmethyl-5-thioxo-1-(4-trifluoromethylsulfanylphenyl)imidazolidin-2-one trifluoroacetate

The compound is prepared from 0.04 mmol of resin, 0.12 mmol of N-Fmoc-L-Ala(OH), 0.20 mmol of 4-pyridinecarboxaldehyde and 0.10 mmol of 4-(trifluoromethanethio)aniline, in the same way as in Example 1-2. After purification by preparative LC-MS, 1.7 mg of expected product are obtained.

EIMS ([M+H]+): 398

RT=4.51 min

### Example 1-15: (S)-4-methyl-3-pyrid-4-ylmethyl-5-thioxo-1-(4-trifluoromethanesulfonylphenyl)imidazolidin-2-one trifluoroacetate

The compound is prepared from 0.04 mmol of resin, 0.12 mmol of N-Fmoc-L-Ala(OH), 0.20 mmol of 4-pyridinecarboxaldehyde and 0.10 mmol of 4-(trifluoro-methanesulfonyl)aniline, in the same way as in Example 1-2. After purification by preparative LC-MS, 2.2 mg of expected product are obtained.

EIMS ([M+H]+): 430

RT=4.34 min

### Example 1-16: (R)-4-methyl-3-(3-methylpyrid-4-ylmethyl)-5-thioxo-1-(4-trifluoro-methylsulfanylphenyl)imidazolidin-2-one trifluoroacetate

The compound is prepared from 0.04 mmol of resin, 0.12 mmol of N-Fmoc-D-Ala(OH), 0.20 mmol of 3-methyl-4-pyridinecarboxaldehyde and 0.10 mmol of 4-(trifluoromethanethio)aniline, in the same way as in Example 1-2. After purification by preparative LC-MS, 1.9 mg of expected product are obtained.

EIMS ([M+H]+): 412

RT=4.60 min

### Example 1-17: (R)-4-methyl-3-(3-methylpyrid-4-ylmethyl)-5-thioxo-1-(4-trifluoro-methylsulfonylphenyl)imidazolidin-2-one trifluoroacetate

The compound is prepared from 0.04 mmol of resin, 0.12 mmol of N-Fmoc-D-Ala(OH), 0.20 mmol of 3-methyl-4-pyridinecarboxaldehyde and 0.10 mmol of 4-(trifluoromethanesulfonyl)aniline, in the same way as in Example 1-2. After purification by preparative LC-MS, 4.5 mg of expected product are obtained.

EIMS ([M+H]+): 444

RT=4.41 min

### Example 1-18: (R)-5-methyl-1-(3-methylpyrid-4-ylmethyl)-3-(4-trifluoromethylsulfanylphenyl)imidazolidine-2,4-dione trifluoroacetate

The compound is prepared from 0.04 mmol of resin, 0.12 mmol of N-Fmoc-D-Ala(OH), 0.20 mmol of 3-methyl-4-pyridinecarboxaldehyde and 0.10 mmol of 4-(trifluoromethanethio)aniline, in the same way as in Example 1-1. After purification by preparative LC-MS, 14 mg of expected product are obtained.

EIMS ([M+H]+): 396

RT=4.22 min

### Example 1-19: (R)-5-methyl-1-(3-methylpyrid-4-ylmethyl)-3-(4-trifluoromethane-sulfonylphenyl)imidazolidine-2,4-dione trifluoroacetate

The compound is prepared from 0.04 mmol of resin, 0.12 mmol of N-Fmoc-D-Ala(OH), 0.20 mmol of 3-methyl-4-pyridinecarboxaldehyde and 0.10 mmol of 4-(trifluoromethanesulfonyl)aniline, in the same way as in Example 1-1. After purification by preparative LC-MS, 6.3 mg of expected product are obtained.

EIMS ([M+H]+): 428

RT=4.10 min

### Example 1-20: (R)-4-methyl-3-quinol-4-ylmethyl-5-thioxo-1-(4-trifluoromethylsulfanylphenyl)imidazolidin-2-one trifluoroacetate

The compound is prepared from 0.04 mmol of resin, 0.12 mmol of N-Fmoc-D-Ala(OH), 0.20 mmol of 4-quinolinecarboxaldehyde and 0.10 mmol of 4-(trifluoromethanethio)aniline, in the same way as in Example 1-2. After purification by preparative LC-MS, 0.4 mg of expected product is obtained.

EIMS ([M+H]+): 448

RT=4.89 min

### Example 1-21: (R)-5-isopropyl-1-quinol-4-ylmethyl-3-(4-trifluoromethylsulfanylphenyl)imidazolidine-2,4-dione trifluoroacetate

The compound is prepared from 0.05 mmol of resin, 0.15 mmol of N-Fmoc-D-Val(OH), 0.25 mmol of 4-quinolinecarboxaldehyde and 0.125 mmol of 4-(trifluoromethanethio)aniline, in the same way as in Example 1-1. After purification by preparative LC-MS, 0.3 mg of expected product is obtained.

RT=4.01 min

EIMS ([M+H]+): 460

### Example 1-22: (R)-5-isopropyl-1-quinol-4-ylmethyl-3-(4-trifluoromethane-sulfonylphenyl)imidazolidine-2,4-dione trifluoroacetate

The compound is prepared from 0.05 mmol of resin, 0.15 mmol of N-Fmoc-D-Val(OH), 0.25 mmol of 4-quinolinecarboxaldehyde and 0.125 mmol of 4-(trifluoromethanesulfonyl)aniline, in the same way as in Example 1-1. After purification by preparative LC-MS, 1 mg of expected product is obtained.

EIMS ([M+H]+): 492

RT=3.91 min

### Example 1-23: (R)-5-Benzyl-1-quinol-4-ylmethyl-3-(4-trifluoromethylsulfanylphenyl)imidazolidine-2,4-dione trifluoroacetate

The compound is prepared from 0.05 mmol of resin, 0.15 mmol of N-Fmoc-D-Phe(OH), 0.25 mmol of 4-quinolinecarboxaldehyde and 0.125 mmol of 4-(trifluoro-methanethio)aniline, in the same way as in Example 1-1. After purification by preparative LC-MS, 8.9 mg of expected product are obtained.

EIMS ([M+H]+): 508

RT=4.11 min

### Example 1-24: (R)-5-Benzyl-1-quinol-4-ylmethyl-3-(4-trifluoromethanesulfonylphenyl)imidazolidine-2,4-dione trifluoroacetate

The compound is prepared from 0.05 mmol of resin, 0.15 mmol of N-Fmoc-D-Phe(OH), 0.25 mmol of 4-quinolinecarboxaldehyde and 0.125 mmol of 4-(trifluoromethanesulfonyl)aniline, in the same way as in Example 1-1. After purification by preparative LC-MS, 5.9 mg of expected product are obtained.

EIMS ([M+H]+): 540

RT=4.01 min

### Example 1-25: (R)-5-Benzyl-1-pyrid-4-ylmethyl-3-(4-trifluoromethanesulfonylphenyl)imidazolidine-2,4-dione trifluoroacetate

The compound is prepared from 0.05 mmol of resin, 0.15 mmol of N-Fmoc-D-Phe(OH), 0.25 mmol of 4-pyridinecarboxaldehyde and 0.125 mmol of 4-(trifluoromethanesulfonyl)aniline, in the same way as in Example 1-1. After purification by preparative LC-MS, 11.1 mg of expected product are obtained.

EIMS ([M+H]+): 490

RT=3.76 min

### Example 1-26: (R)-5-isobutyl-1-quinol-4-ylmethyl-3-(4-trifluoromethanesulfonylphenyl)imidazolidine-2,4-dione trifluoroacetate

The compound is prepared from 0.05 mmol of resin, 0.15 mmol of N-Fmoc-D-Leu(OH), 0.25 mmol of 4-quinolinecarboxaldehyde and 0.125 mmol of 4-(trifluoromethane-sulfonyl)aniline, in the same way as in Example 1-1. After purification by preparative LC-MS, 5.9 mg of expected product are obtained.

RT=4.02 min

EIMS ([M+H]+): 506

### Example 1-27: (R)-5-(4-hydroxybenzyl)-1-quinol-4-ylmethyl-3-(4-trifluoromethyl-sulfanylphenyl)imidazolidine-2,4-dione trifluoroacetate

The compound is prepared from 0.05 mmol of resin, 0.15 mmol of N-Fmoc-D-Tyr(OH), 0.25 mmol of 4-quinolinecarboxaldehyde and 0.125 mmol of 4-(trifluoromethanethio)aniline, in the same way as in Example 1-1. After purification by preparative LC-MS, 5.1 mg of expected product are obtained.

EIMS ([M+H]+): 524

RT=3.75 min

### Example 1-28: (R)-5-(4-hydroxybenzyl)-1-quinol-4-ylmethyl-3-(4-trifluoromethanesulfonylphenyl) imidazolidine-2,4-dione trifluoroacetate

The compound is prepared from 0.05 mmol of resin, 0.15 mmol of N-Fmoc-D-Tyr(OH), 0.25 mmol of 4-quinolinecarboxaldehyde and 0.125 mmol of 4-(trifluoro-methanesulfonyl)aniline, in the same way as in Example 1-1. After purification by preparative LC-MS, 1.8 mg of expected product are obtained.

EIMS ([M+H]+): 556

RT=3.66 min

### Example 1-29: (R)-5-(4-hydroxybenzyl)-1-pyrid-4-ylmethyl-3-(4-trifluoromethane-sulfonylphenyl)imidazolidine-2,4-dione trifluoroacetate

The compound is prepared from 0.05 mmol of resin, 0.15 mmol of N-Fmoc-D-Tyr(OH), 0.25 mmol of 4-pyridinecarboxaldehyde and 0.125 mmol of 4-(trifluoromethanesulfonyl)aniline, in the same way as in Example 1-1. After purification by preparative LC-MS, 0.7 mg of expected product is obtained.

EIMS ([M+H]+): 506

RT=3.48 min

### Example 1-30: (R)-5-(1-hydroxyethyl)-1-quinol-4-ylmethyl-3-(4-trifluoromethyl-sulfanylphenyl)imidazolidine-2,4-dione trifluoroacetate

The compound is prepared from 0.05 mmol of resin, 0.15 mmol of N-Fmoc-D-Thr(OH), 0.25 mmol of 4-quinolinecarboxaldehyde and 0.125 mmol of 4-(trifluoromethanethio)aniline, in the same way as in Example 1-1. After purification by preparative LC-MS, 4.2 mg of expected product are obtained.

EIMS ([M+H]+): 462

RT=3.52 min

### Example 1-31: (R)-5-(1-hydroxyethyl)-1-quinol-4-ylmethyl-3-(4-trifluoromethane-sulfonylphenyl)imidazolidine-2,4-dione trifluoroacetate

The compound is prepared from 0.05 mmol of resin, 0.15 mmol of N-Fmoc-D-Thr(OH), 0.25 mmol of 4-quinolinecarboxaldehyde and 0.125 mmol of 4-(trifluoro-methanesulfonyl)aniline, in the same way as in Example 1-1. After purification by preparative LC-MS, 3.4 mg of expected product are obtained.

EIMS ([M+H]+): 494

RT=3.43 min

### Example 1-32: 4-quinol-4-ylmethyl-6-(4-trifluoromethylsulfanylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 4-quinolinecarboxaldehyde and 0.125 mmol of 4-(trifluoromethane-thio)aniline, in the same way as in Example 1-1. After purification by preparative LC-MS, 7.5 mg of expected product are obtained.

EIMS ([M+H]+): 444

RT=3.68 min

### Example 1-33: 4-quinol-4-ylmethyl-6-(4-trifluoromethanesulfonylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 4-quinolinecarboxaldehyde and 0.125 mmol of 4-(trifluoromethane-sulfonyl)aniline, in the same way as in Example 1-1. After purification by preparative LC-MS, 3.8 mg of expected product are obtained.

EIMS ([M+H]+): 476

RT=3.60 min

### Example 1-34: 4-pyrid-4-ylmethyl-6-(4-trifluoromethylsulfanylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 4-pyridinecarboxaldehyde and 0.125 mmol of 4-(trifluoromethane-thio)aniline, in the same way as in Example 1-1. After purification by preparative LC-MS, 1.7 mg of expected product are obtained.

EIMS ([M+H]+): 394

RT=3.43 min

### Example 1-35: 4-pyrid-4-ylmethyl-6-(4-trifluoromethanesulfonylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 4-pyridinecarboxaldehyde, and 0.125 mmol of 4-(trifluoromethane-sulfonyl)aniline, in the same way as in Example 1-1. After purification by preparative LC-MS, 1.4 mg of expected product are obtained.

EIMS ([M+H]+): 426

RT=3.35 min

### Example 1-36: (R)-5-benzo[b]thiophen-3-ylmethyl-1-quinol-4-ylmethyl-3-(4-trifluoromethanesulfonylphenyl)imidazolidine-2,4-dione trifluoroacetate

The compound is prepared from 0.05 mmol of resin, 0.15 mmol of N-Fmoc-D-benzothienylAla(OH), 0.25 mmol of 4-quinolinecarboxaldehyde and 0.125 mmol of 4-(trifluoromethanesulfonyl)aniline, in the same way as in Example 1-1. After purification by preparative LC-MS, 5 mg of expected product are obtained.

EIMS ([M+H]+): 596

RT=4.12 min

### Example 1-37: (R)-5-benzo[b]thiophen-3-ylmethyl-1-pyrid-4-ylmethyl-3-(4-trifluoromethylsulfanylphenyl)imidazolidine-2,4-dione trifluoroacetate

The compound is prepared from 0.05 mmol of resin, 0.15 mmol of N-Fmoc-D-benzothienylAla(OH), 0.25 mmol of 4-pyridinecarboxaldehyde and 0.125 mmol of 4-(trifluoromethanethio)aniline, in the same way as in Example 1-1. After purification by preparative LC-MS, 1.4 mg of expected product are obtained.

EIMS ([M+H]+): 514

RT=3.35 min

### Example 1-38: (R)-5-benzo[b]thiophen-3-ylmethyl-1-pyrid-4-ylmethyl-3-(4-trifluoromethanesulfonylphenyl)imidazolidine-2,4-dione trifluoroacetate

The compound is prepared from 0.05 mmol of resin, 0.15 mmol of N-Fmoc-D-benzothienylAla(OH), 0.25 mmol of 4-pyridinecarboxaldehyde and 0.125 mmol of 4-(trifluoromethanesulfonyl)aniline, in the same way as in Example 1-1. After purification by preparative LC-MS, 8.5 mg of expected product are obtained.

EIMS ([M+H]+): 546

RT=3.90 min

### Example 1-39: (S)-5-pyrid-2-ylmethyl-1-quinol-4-ylmethyl-3-(4-trifluoro-methylsulfanylphenyl)imidazolidine-2,4-dione trifluoroacetate

The compound is prepared from 0.05 mmol of resin, 0.15 mmol of N-Fmoc-D-2-pyridine-Ala(OH), 0.25 mmol of 4-quinolinecarboxaldehyde and 0.125 mmol of 4-(tri-fluoromethanethio)aniline, in the same way as in Example 1-1. After purification by preparative LC-MS, 7.5 mg of expected product are obtained.

EIMS ([M+H]+): 509

RT=3.47 min

### Example 1-40: (S)-5-pyrid-2-ylmethyl-1-quinol-4-ylmethyl-3-(4-trifluoro-methanesulfonylphenyl)imidazolidine-2,4-dione trifluoroacetate

The compound is prepared from 0.05 mmol of resin, 0.15 mmol of N-Fmoc-D-2-pyridine-Ala(OH), 0.25 mmol of 4-quinolinecarboxaldehyde and 0.125 mmol of 4-(trifluoromethanesulfonyl)aniline, in the same way as in Example 1-1. After purification by preparative LC-MS, 1.6 mg of expected product are obtained.

EIMS ([M+H]+): 541

RT=3.41 min

### Example 1-41: (R)-1-(3-hydroxypyrid-4-ylmethyl)-5-methyl-3-(4-trifluoro-methylsulfanylphenyl)imidazolidine-2,4-dione trifluoroacetate

The compound is prepared from 0.05 mmol of resin, 0.15 mmol of N-Fmoc-D-Ala(OH), 0.25 mmol of 3-hydroxy-4-pyridinecarboxaldehyde and 0.125 mmol of 4-(trifluoromethanethio)aniline, in the same way as in Example 1-1. After purification by preparative LC-MS, 8.3 mg of expected product are obtained.

EIMS ([M+H]+): 398

RT=4.26 min

### Example 1-42: 5,5-dimethyl-1-quinol-4-ylmethyl-3-(4-trifluoromethoxyphenyl)imidazolidine-2,4-dione trifluoroacetate

The compound is prepared from 0.05 mmol of resin, 0.15 mmol of Fmoc-AIB-(OH), 0.25 mmol of 4-quinolinecarboxaldehyde and 0.125 mmol of 4-(trifluoromethoxy) aniline, in the same way as in Example 1-1. After purification by preparative LC-MS, 5.5 mg of expected product are obtained.

EIMS ([M+H]+): 430

RT=4.33 min

### Example 1-43: 5,5-dimethyl-1-quinol-4-ylmethyl-3-(4-trifluoromethylsulfanylphenyl)imidazolidine-2,4-dione trifluoroacetate

The compound is prepared from 0.05 mmol of resin, 0.15 mmol of Fmoc-AIB-(OH), 0.25 mmol of 4-quinolinecarboxaldehyde, and 0.125 mmol of 4-(trifluoro-methanethio)aniline, in the same way as in Example 1-1. After purification by preparative LC-MS, 9.4 mg of expected product are obtained.

EIMS ([M+H]+): 446

RT=4.58 min

### Example 1-44: 5,5-dimethyl-1-(3-methylpyrid-4-ylmethyl)-3-(4-trifluoromethoxyphenyl)imidazolidine-2,4-dione trifluoroacetate

The compound is prepared from 0.05 mmol of resin, 0.15 mmol of Fmoc-AIB-(OH), 0.25 mmol of 3-methyl-4-pyridinecarboxaldehyde and 0.125 mmol of 4-(trifluoro-methoxy)aniline, in the same way as in Example 1-1. After purification by preparative LC-MS, 5.5 mg of expected product are obtained.

EIMS ([M+H]+): 394

RT=4.06 min

### Example 1-45: 5,5-dimethyl-1-(3-methylpyrid-4-ylmethyl)-3-(4-trifluoromethylsulfanylphenyl)imidazolidine-2,4-dione trifluoroacetate

The compound is prepared from 0.05 mmol of resin, 0.15 mmol of Fmoc-AIB-(OH), 0.25 mmol of 3-methyl-4-pyridinecarboxaldehyde and 0.125 mmol of 4-(trifluoro-methanethio)aniline, in the same way as in Example 1-1. After purification by preparative LC-MS, 13.1 mg of expected product are obtained.

EIMS ([M+H]+): 410

RT=4.30 min

### Example 1-46: 5,5-dimethyl-1-(3-methylpyrid-4-ylmethyl)-3-(4-trifluoromethane-sulfonylphenyl)imidazolidine-2,4-dione trifluoroacetate

The compound is prepared from 0.05 mmol of resin, 0.15 mmol of Fmoc-AIB-(OH), 0.25 mmol of 3-methyl-4-pyridinecarboxaldehyde and 0.125 mmol of 4-(trifluoro-methanesulfonyl)aniline, in the same way as in Example 1-1. After purification by preparative LC-MS, 9.6 mg of expected product are obtained.

EIMS ([M+H]+): 442

RT=4.18 min

### Example 1-47: 1-(3-hydroxypyrid-4-ylmethyl)-5,5-dimethyl-3-(4-trifluoromethoxyphenyl)imidazolidine-2,4-dione trifluoroacetate

The compound is prepared from 0.05 mmol of resin, 0.15 mmol of Fmoc-AIB-(OH), 0.25 mmol of 3-hydroxy-4-pyridinecarboxaldehyde and 0.125 mmol of 4-(trifluoro-methoxy)aniline, in the same way as in Example 1-1. After purification by preparative LC-MS, 6.9 mg of expected product are obtained.

EIMS ([M+H]+): 394

RT=4.15 min

### Example 1-48: 1-(3-hydroxypyrid-4-ylmethyl)-5,5-dimethyl-3-(4-trifluoromethyl-sulfanylphenyl)imidazolidine-2,4-dione trifluoroacetate

The compound is prepared from 0.05 mmol of resin, 0.15 mmol of Fmoc-AIB-(OH), 0.25 mmol of 3-hydroxy-4-pyridinecarboxaldehyde and 0.125 mmol of 4-(trifluoromethanethio)aniline, in the same way as in Example 1-1. After purification by preparative LC-MS, 9.7 mg of expected product are obtained.

EIMS ([M+H]+): 412

RT=4.39 min

### Example 1-49: 1-(3-hydroxypyrid-4-ylmethyl)-5,5-dimethyl-3-(4-trifluoromethane-sulfonylphenyl)imidazolidine-2,4-dione trifluoroacetate

The compound is prepared from 0.05 mmol of resin, 0.15 mmol of Fmoc-AIB-(OH), 0.25 mmol of 3-hydroxy-4-pyridinecarboxaldehyde and 0.125 mmol of 4-(trifluoromethanesulfonyl)aniline, in the same way as in Example 1-1. After purification by preparative LC-MS, 15.2 mg of expected product are obtained.

EIMS ([M+H]+): 444

RT=4.30 min

### Example 1-50: 4-quinol-4-ylmethyl-6-(4-trifluoromethoxyphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 4-quinolinecarboxaldehyde and 0.125 mmol of 4-(trifluoromethoxy)-aniline, in the same way as in Example 1-1. After purification by preparative LC-MS, 4.1 mg of expected product are obtained.

EIMS ([M+H]+): 428

RT=4.24 min

### Example 1-51: 4-(3-methylpyrid-4-ylmethyl)-6-(4-trifluoromethoxyphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 4-pyridinecarboxaldehyde and 0.125 mmol of 4-(trifluoromethoxy)-aniline, in the same way as in Example 1-1. After purification by preparative LC-MS, 3 mg of expected product are obtained.

EIMS ([M+H]+): 392

RT=3.95 min

### Example 1-52: 4-(3-methylpyrid-4-ylmethyl)-6-(4-trifluoromethylsulfanylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 3-methyl-4-pyridinecarboxaldehyde and 0.125 mmol of 4-(trifluoro-methanethio)aniline, in the same way as in Example 1-1. After purification by preparative LC-MS, 5 mg of expected product are obtained.

EIMS ([M+H]+): 408

RT=4.24 min

### Example 1-53: of 4-(3-methylpyrid-4-ylmethyl)-6-(4-trifluoromethanesulfonylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 3-methyl-4-pyridinecarboxaldehyde and 0.125 mmol of 4-(trifluoro-methanesulfonyl)aniline, in the same way as in Example 1-1. After purification by preparative LC-MS, 1.9 mg of expected product are obtained.

EIMS ([M+H]+): 440

RT=4.11 min

### Example 1-54: 4-(3-hydroxypyrid-4-ylmethyl)-6-(4-trifluoromethoxyphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 3-hydroxy-4-pyridinecarboxaldehyde and 0.125 mmol of 4-(trifluoromethoxy)aniline, in the same way as in Example 1-1. After purification by preparative LC-MS, 5.5 mg of expected product are obtained.

EIMS ([M+H]+): 394

RT=4.04 min

### Example 1-55: 4-(3-hydroxypyrid-4-ylmethyl)-6-(4-trifluoromethylsulfanylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 3-hydroxy-4-pyridinecarboxaldehyde and 0.125 mmol of 4-(trifluoromethanethio)aniline, in the same way as in Example 1-1. After purification by preparative LC-MS, 0.7 mg of expected product is obtained.

EIMS ([M+H]+): 410

RT=4.34 min

### Example 1-56: 4-(3-hydroxypyrid-4-ylmethyl)-6-(4-trifluoromethanesulfonylphenyl)-4,6-diazaspiro[2.4]heptane-5,7-dione trifluoroacetate

The compound is prepared from 0.05 mmol of resin, 0.15 mmol of Fmoc-ACPC-(OH), 0.25 mmol of 3-hydroxy-4-pyridinecarboxaldehyde and 0.125 mmol of 4-(trifluoromethanesulfonyl)aniline, in the same way as in Example 1-1. After purification by preparative LC-MS, 2.5 mg of expected product are obtained.

EIMS ([M+H]+): 442

RT=4.17 min

### Example 1-57: 5-methyl-1-quinol-4-ylmethyl-3-(4-trifluoromethanesulfonylphenyl)dihydropyrimidine-2,4-dione

588 mg (1 mmol) of Wang polystyrene resin (1.7 mmol/g) are washed with 2×5 ml of DMF, 1×5 ml of DCM and then treated with a solution of 0.49 g of N-Fmoc-3-amino-2-(R,S)-methylpropionic acid (1.5 mmol), 0.24 g of pyridine (3 mmol) in 5 ml of DMF, immediately followed by 0.31 g of 2,6-dichlorobenzoyl chloride (dropwise addition to control the exothermicity). The reaction mixture is stirred overnight at room temperature. The mixture is filtered and the resin is then washed with 1×5 ml of DMF, 1×5 ml of DCM and 2×5 ml of DMF and then treated with 5 ml of a 10% solution of piperidine in DMF. The resin is then washed with 2×5 ml of DMF, 1×5 ml of DCM, 1×5 ml of DMF, 1×5 ml of DMF, 4×5 ml of DCM, 4×5 ml of MeOH and dried under vacuum. 0.94 g of 4-quinolinecarboxaldehyde (6 mol) in 16 ml of a 50/50 mixture of THF/TMOF is added to the resin, which is stirred overnight. The resin is then washed 3 times with 5 ml of the same THF/TMOF mixture and then treated with 12 ml of a 1M solution of sodium cyanoborohydride in THF (12 mmol) in the presence of 1.2 ml of MeOH and 0.12 ml of acetic acid. After stirring overnight at room temperature, the resin is washed with 1×5 ml of THF, 4×5 ml of a 30% solution of acetic acid in DMF, 1×5 ml of MeOH, 1×5 ml of THF, 1×5 ml of DMF, 1×5 ml of THF, 1×5 ml of MeOH and dried under vacuum.

In parallel, 0.121 g of triphosgene (0.41 mmol) in 1 ml of DCM is added dropwise to a solution of 0.281 g of 4-(trifluoromethanesulfonyl)aniline (1.25 mmol) and 0.1 g of pyridine (1.25 mmol) in 2 ml of DCM. After stirring for 15 minutes at room temperature, the same amount of pyridine is added, followed by the resin previously prepared. The mixture is stirred overnight and then filtered. The resin is washed with 1×5 ml of MeOH, 1×5 ml of THF, 1×5 ml of MeOH, 1×5 ml of DMF, 1×5 ml of THF, 1×5 ml of MeOH and 3×5 ml of THF and dried under vacuum. The resin is then treated with 154 mg of DBU (1 mmol) in 5 ml of DCM and stirred overnight. Finally, the dihydrouracil is obtained by treating the resin with 5 ml of 2% solution of acetic acid in THF. After purification by preparative HPLC, 90 mg of expected product are isolated.

EIMS ([M+H]+): 477

RT=1.83 min (20-100% ACN/H2O gradient over 5 minutes)

### Example 1-58: (S)-5-Methyl-1-quinol-4-ylmethyl-3-(4-trifluoromethanesulfanylphenyl)imidazolidine-2,4-dione

This example describes a novel preparation of Example 1-5 above.

A mixture of 0.71 g of ethyl (S)-2-[(quinol-4-ylmethyl)amino]propanoate and 1.42 g of 4-(trifluoromethanesulfanylphenyl) isocyanate in 15 ml of THF is stirred for 15 hours at room temperature under an argon atmosphere. After evaporating off the solvent under reduced pressure, 20 ml of dichloromethane are added. The precipitate is filtered off. The filtrate is concentrated under reduced pressure and the residue is purified by flash chromatography (SiO2, CH2Cl2 and then CH2Cl2/MeOH, 95/5 by volume as eluent, Ar). 0.69 g of (S)-5-methyl-1-quinol-4-ylmethyl-3-(4-trifluoromethanesulfanylphenyl)imidazolidine-2,4-dione is isolated in the form of a white powder.

[α]D=−33.1°+/−0.8° (MeOH)

Mass: EI m/z=431 M+. base peak

m/z=143 [C10H9N]+.

1H NMR spectrum (400 MHz, (CD3)2SO d6, δ in ppm): 1.42 (d, J=5.5 Hz: 3H); 4.35 (q, J=5.5 Hz: 1H); 5.08 (d, J=17 Hz: 1H); 5.25 (d, J=17 Hz: 1H); 7.65 (d, J=5 Hz: 1H); from 7.65 to 7.75 (mt: 1H); 7.70 (d, J=8.5 Hz: 2H); 7.83 (broad t, J=8 Hz: 1H); 7.89 (d, J=8.5 Hz: 2H); 8.10 (broad d, J=8.5 Hz: 1H); 8.25 (broad d, J=8.5 Hz: 1H); 8.90 (d, J=5 Hz: 1H).

### Ethyl (S)-2-[(quinol-4-ylmethyl)amino]propanoate (P-31397-073-1)

A mixture of 2 g of L-alanine ethyl ester in hydrochloride form and 1.83 ml of triethylamine in 30 ml of dichloromethane is stirred at room temperature for 10 minutes. Next, 2.05 g of quinoline-4-carbaldehyde are added. The reaction medium is stirred at room temperature for 15 hours and then concentrated under reduced pressure. 35 ml of ethanol are then added; the solution is cooled to 0° C. and 0.49 g of sodium borohydride is then added portionwise. Stirring is continued for 15 hours at room temperature. The precipitate formed is filtered off; the filtrate is concentrated under reduced pressure. The residue is purified by flash chromatography on a column (SiO2, CH2Cl2/MeOH, 95/5 by volume as eluent, Ar). 0.71 g of ethyl (S)-2-[(quinol-4-ylmethyl)amino]propanoate is obtained in the form of a pink oil.

Mass: EI m/z=258 M+.

m/z=185 [M-COOCH2CH3]+ base peak

m/z=142 [C10H8N]+

1H NMR spectrum (300 MHz, (CD3)2SO d6, δ in ppm): 1.22 (t, J=7 Hz: 3H); 1.28 (d, J=7 Hz: 3H); 2.72 (unresolved peak: 1H); 3.42 (mt: 1H); from 4.00 to 4.20 (mt: 1H); 4.13 (q, J=7 Hz: 2H); 4.27 (broad d, J=16 Hz: 1H); 7.55 (broad d, J=5 Hz: 1H); 7.64 (ddd, J=8.5-7.5 and 1 Hz: 1H); 7.77 (ddd, J=8.5-7.5 and 1 Hz: 1H); 8.04 (broad d, J=8.5 Hz: 1H); 8.22 (broad d, J=8.5 Hz: 1H); 8.86 (d, J=5 Hz: 1H).

### Example 1-59: 5,5-Dimethyl-1-quinol-4-ylmethyl-3-(4-trifluoromethoxyphenyl)imidazolidine-2,4-dione

This example describes a novel preparation of Example 1-42 above.

The product is prepared according to the procedure described in Example 1-58 with 600 mg of methyl 2-methyl-2-[(quinol-4-ylmethyl)amino]propanoate instead of ethyl (S)-2-[(quinol-4-ylmethyl)amino]propanoate used in Example 1-58 and 1.114 g of 4-(trifluoromethoxyphenyl) isocyanate instead of 4-(trifluoromethanesulfanylphenyl) isocyanate used in Example 1-58. After purification by flash chromatography on a column (SiO2, CH2Cl2 as eluent, Ar) and then a second purification by flash chromatography on a column (SiO2, 60/40 cyclohexane/EtOAc by volume as eluent, Ar), 710 mg of the desired product are obtained.

Mass: EI m/z=429 M+. base peak

m/z=414 [M-CH3]+

m/z=359 [M-C4H6O]+.

1H NMR spectrum (300 MHz, (CD3)2SO d6, δ in ppm): 1.46 (s: 6H); 5.16 (s: 2H); 7.55 (broad d, J=8.5 Hz: 2H); 7.65 (d, J=5 Hz: 1H); 7.69 (d, J=8.5 Hz: 2H); 7.70 (mt: 1H); 7.83 (ddd, J=8-7.5 and 1.5 Hz: 1H); 8.09 (broad d, J=8.5 Hz: 1H); 8.27 (broad d, J=8.5 Hz: 1H); 8.88 (d, J=5 Hz: 1H).

### Preparation of methyl 2-((quinol-4-ylmethyl)amino)propanoate

A mixture of 1.5 g of methyl o-aminoisobutyrate hydrochloride and 1.4 ml of triethylamine in 30 ml of dichloromethane is stirred at 0° C. for 20 minutes. Next, 1 g of magnesium sulfate and 1.5 g of quinoline-4-carbaldehyde are added. Stirring is continued for 15 hours at room temperature and the mixture is then concentrated under reduced pressure. The residue is taken up in 35 ml of methanol and the solution obtained is cooled to 0° C. 0.4 g of sodium borohydride is added portion-wise and stirring is continued at room temperature for 15 hours. The precipitate formed is filtered and the filtrate is concentrated under reduced pressure. The residue obtained is purified by recrystallization from diisopropyl ether. 600 mg of the expected product are obtained in the form of a pink oil.

Mass: DCI m/z=259 [M+H]+

m/z=199 [M+H-HCOOCH3]+

1H NMR spectrum (300 MHz, (CD3)2SO d6, δ in ppm): 1.36 (s: 6H); 2.68 (broad t, J=7 Hz: 1H); 3.69 (s: 3H); 4.11 (d, J=7 Hz: 2H); 7.60 (broad d, J=5 Hz: 1H); 7.63 (ddd, J=9-8.5 and 1 Hz: 1H); 7.76 (ddd, J=9-8.5 and 1 Hz: 1H); 8.03 (broad d, J=8.5 Hz: 1H); 8.20 (broad d, J=8.5 Hz: 1H); 8.85 (d, J=5 Hz: 1H).

### Example 1-60: 5,5-dimethyl-1-(3-chloro-6-methoxyquinol-4-ylmethyl)-3-(4-trifluoromethoxyphenyl)imidazolidine-2,4-dione

The product is prepared according to the procedure described in Example 1-59, starting with 180 mg of methyl 2-methyl-2-[(3-chloro-6-methoxyquinol-4-ylmethyl)amino]propanoate instead of methyl 2-methyl-2-[(quinol-4-ylmethyl)amino]propanoate used in Example 1-59 and 267 mg of 4-(trifluoromethoxy)phenyl isocyanate. After purification by flash chromatography on a column (SiO2, 80/20 cyclohexane/EtOAc by volume as eluent, Ar), 137 mg of the expected product are obtained.

Mass: EI m/z=493 M+. isotopic band of the peak

monochloro m/z=458 [M-Cl]+ base peak

1H NMR spectrum (300 MHz, (CD3)2SO d6, δ in ppm): 1.27 (s: 6H); 3.89 (s: 3H); 5.27 (s: 2H); 7.48 (dd, J=9 and 3 Hz: 1H); 7.56 (broad d, J=8.5 Hz: 2H); 7.68 (dt, J=8.5 and 2 Hz: 2H); 7.79 (d, J=3 Hz: 1H); 8.01 (d, J=9 Hz: 1H); 8.80 (s: 1H).

### Preparation of methyl 2-methyl-2-[(3-chloro-6-methoxyquinol-4-ylmethyl)amino]propanoate (P-31397-099-1)

The product is prepared according to the procedure described in Example 59, starting with 1 g of methyl α-aminoisobutyrate hydrochloride, 1.25 g of (3-chloro-6-methoxy)quinoline-4-carbaldehyde instead of quinoline-4-carbaldehyde used in Example 1-59, 0.66 g of triethylamine and 250 mg of sodium borohydride. After purification by flash chromatography (SiO2, 70/30 cyclohexane/EtOAc by volume as eluent, Ar), 180 mg of the expected product are obtained.

Mass: EI m/z=322 M+. isotopic band of the peak

monochloro m/z=263 [M-COOCH3]+ base peak

isotopic band of the monochloro peak m/z=206 [M-C₅H₁₀O₂N]+

isotopic band of the monochloro peak

1H NMR spectrum (300 MHz, (CD3)2SO d6, δ in ppm): 1.39 (s: 6H); 2.47 (broad t, J=7.5 Hz: 1H); 3.74 (s: 3H); 3.98 (s: 3H); 4.04 (d, J=7.5 Hz: 2H); 7.46 (dd, J=9 and 3 Hz: 1H); 7.66 (d, J=3 Hz: 1H); 7.97 (d, J=9 Hz: 1H); 8.71 (s: 1H).

### Preparation of (3-chloro-6-methoxyquinoline)-4-carbaldehyde (P-31397-097-1)

A solution of 2 g of 4-bromo-3-chloro-6-methoxyquinoline in 50 ml of THF is cooled to −78° C. 6.9 ml of a 1.6 M solution of nBuLi in dioxane are added. The solution is stirred for 2 hours at this temperature and 1.7 ml of DMF are then added. The mixture is stirred at −60° C. for 2 hours 30 minutes and the reaction medium is then allowed to warm to room temperature. 200 ml of water are then added. The organic phase is extracted with 200 ml of ethyl acetate, washed with 5×200 ml of water, dried over magnesium sulfate and concentrated under reduced pressure. The residue obtained is purified by flash chromatography on a column (SiO2, 80/20 cyclohexane/EtOAc by volume as eluent, Ar). 1.2 g of the expected product are obtained in the form of a yellow powder.

Mass: EI m/z=221 M+. base peak, isotopic band of the monochloro

m/z=193 [M-CO]+. isotopic band of the monochloro peak

m/z=150 [M-C3H3O2]+ isotopic band of the monochloro peak

1H NMR spectrum (300 MHz, (CD3)2SO d6, δ in ppm): 3.96 (s: 3H); 7.56 (dd, J=9 and 3 Hz: 1H); 8.07 (d, J=9 Hz: 1H); 8.22 (d, J=3 Hz: 1H); 8.93 (s: 1H); 10.77 (s: 1H).

Preparation of 4-bromo-3-chloro-6-methoxyquinoline is described in French patent FR 2 816 618 in Example 1-1.

### Example 1-61: 5,5-Dimethyl-1-pyrid-4-ylmethyl-3-(4-trifluoro-methanesulfanylphenyl)imidazolidine-2,4-dione

0.764 g of 4-(trifluoromethanesulfanylphenyl) isocyanate is added to a solution of 0.726 g of methyl 2-methyl-2-[(pyrid-4-ylmethyl)amino]propanoate in 10 ml of tetrahydrofuran. The reaction medium is stirred under an argon atmosphere for about 3 days at a temperature in the region of 20° C. The reaction mixture is taken up in ethyl acetate, washed successively with water and then with saturated sodium chloride solution. The organic phase is dried over magnesium sulfate and then concentrated under reduced pressure. The residue thus obtained is purified by flash chromatography on an AIT cartridge of reference FC-50SI filled with 50 g of silica conditioned and eluted with dichloromethane at a flow rate of 10 ml per minute. The fractions between 100 and 280 ml are concentrated under reduced pressure, the residue obtained is taken up in ethyl ether and the insoluble material is filtered off. 700 mg of 5,5-dimethyl-1-pyrid-4-ylmethyl-3-(4-trifluoromethanesulfanylphenyl)imidazolidine-2,4-dione are thus obtained in the form of a white powder, the characteristics of which are as follows:

1H NMR spectrum (300 MHz, (CD3)2SO d6, δ in ppm): 1.43 (s: 6H); 4.66 (s: 2H); 7.44 (broad d, J=6 Hz: 2H); 7.69 (d t, J=8.5 and 2.5 Hz: 2H); 7.87 (broad d, J=8.5 Hz: 2H); 8.55 (dd, J=6 and 1.5 Hz: 2H).

Mass IE m/z=395 M+. base peak

m/z=380 (M-CH3)+

m/z=219 C8H4NOSF3+.

m/z=92 C6H6N+

### Preparation of methyl 2-methyl-2-[(pyrid-4-ylmethyl)amino]propanoate (P-31402-151-1)

1.04 ml of triethylamine and then 0.659 g of pyridine-4-carbaldehyde are successively added to a solution of 0.945 g of methyl α-aminoisobutyrate hydrochloride in 28 ml of dichloroethane. The reaction mixture is stirred overnight at a temperature in the region of 20° C. The mixture is purified by filtration on Merck Lichroprep aminopropyl-grafted silica. The filtrate is concentrated under reduced pressure and the residue thus obtained is taken up in 25 ml of methanol, 0.372 g of sodium borohydride is added. The reaction mixture is stirred for 48 hours at a temperature in the region of 20° C. and then poured into a mixture of a normal solution of sodium hydroxide/ice. The mixture obtained is extracted three times with ethyl acetate. The organic phase is dried over magnesium sulfate and then concentrated under reduced pressure. 0.726 g of methyl 2-methyl-2-[(pyrid-4-ylmethyl)amino]propanoate is thus obtained in the form of an oil, the characteristics of which are as follows:

1H NMR spectrum (300 MHz, (CD3)2SO d6, δ in ppm): 1.27 (s: 6H); 2.69 (broad t, J=5 Hz: 1H); 3.64 (s: 3H); 3.65 (d, J=5 Hz: 2H); 7.35 (broad d, J=6 Hz: 2H); 8.47 (dd, J=6 and 1.5 Hz: 2H).

Mass IC m/z=209 MH+ base peak

m/z=149 (M-C2H4O2)+

### Example 1-62: 1-Pyrid-4-ylmethyl-3-(4-trifluoromethanesulfanylphenyl)imidazolidine-2,4-dione

0.087 g sodium hydride is added to a solution of 0.300 g of 3-(4-trifluoromethyl-sulfanylphenyl)imidazolidine-2,4-dione in 6 ml of anhydrous dimethylformamide, under an inert atmosphere of argon at a temperature in the region of 20° C., stirring is continued at this temperature for 30 minutes, 0.152 ml of triethylamine and 0.274 g of 4-(bromomethyl)pyridine hydrobromide are successively added, followed by addition of ice-cold water 10 minutes later. The reaction mixture is placed on a cartridge 37 mm in diameter packed with 50 g of Amicon 50 μm octadecyl-grafted silica of ref conditioned successively with a water/acetonitrile mixture (5/95, v/v) and then a water/acetonitrile mixture (95/5, v/v). The elution was performed with a water/acetonitrile mixture (95/5, v/v) over 20 minutes, followed by a linear gradient from 5% to 95% of acetonitrile over 60 minutes, at a flow rate of 10 ml/minute. The fractions between 580 and 630 ml are concentrated under reduced pressure. 0.220 g of 1-pyrid-4-ylmethyl-3-(4-trifluoromethylsulfanylphenyl)imidazolidine-2,4-dione is thus obtained in the form of a white powder, the characteristics of which are as follows:

1H NMR spectrum (300 MHz, (CD3)2SO d6, δ in ppm): 4.16 (s: 2H); 4.65 (s: 2H); 7.42 (broad d, J=6 Hz: 2H); 7.64 (broad d, J=8.5 Hz: 2H); 7.87 (broad d, J=8.5 Hz: 2H); 8.57 (broad d, J=6 Hz: 2H).

Mass IE m/z=367 M+. base peak

m/z=219 C8H4NOSF3+.

m/z=92 C6H6N+

The compound 3-(4-trifluoromethylsulfanylphenyl)imidazolidine-2,4-dione is described in patent U.S. Pat. No. 4,496,575.

### Example 1-63: 5,5-Dimethyl-1-pyrid-4-ylmethyl-3-(4-trifluoro-methoxyphenyl)imidazolidine-2,4-dione

0.042 g of sodium hydride is added to a solution of 0.150 g of 5,5-dimethyl-3-(4-trifluoromethoxyphenyl)imidazolidine-2,4-dione in 3 ml of anhydrous dimethylformamide, under an inert atmosphere of argon and at a temperature in the region of 20° C., stirring is continued at this temperature for 30 minutes, 0.094 ml of triethylamine and 0.132 g of 4-(bromomethyl)pyridine hydrobromide are successively added, followed by addition of ice-cold water 10 minutes later. The reaction mixture is placed on a cartridge 27 mm in diameter packed with 30 g of Amicon 50 μm octadecyl-grafted silica conditioned successively with a water/acetonitrile mixture (5/95, v/v) and then a water/acetonitrile mixture (95/5, v/v). The elution was performed with a water/acetonitrile mixture (95/5, v/v) over 20 minutes, followed by a linear gradient from 5% to 95% of acetonitrile over 60 minutes, at a flow rate of 10 ml/minute. The fractions between 300 and 450 ml are concentrated under reduced pressure. 0.1 g of mixture is thus obtained, which is repurified on a cartridge of 37 mm in diameter packed with 50 g of Amicon 50 μm octadecyl-grafted silica of ref. conditioned successively with a water/acetonitrile mixture (5/95, v/v) and then a water/acetonitrile mixture (95/5, v/v). The elution was performed with a water/acetonitrile mixture (95/5, v/v) over 20 minutes, followed by a linear gradient from 5% to 95% of acetonitrile over 60 minutes, at a flow rate of 10 ml/minute. The fractions between 550 and 750 ml are concentrated under reduced pressure. 0.060 g of 5,5-dimethyl-1-pyrid-4-ylmethyl-3-(4-tri-fluoromethoxyphenyl)imidazolidine-2,4-dione is thus obtained, the characteristics of which are as follows:

1H NMR spectrum (300 MHz, (CD3)2SO d6, δ in ppm): 1.42 (s: 6H); 4.65 (s: 2H); 7.44 (broad d, J=6 Hz: 2H); 7.53 (broad d, J=8.5 Hz: 2H); 7.64 (dt, J=8.5 and 2.5 Hz: 2H); 8.54 (broad d, J=6 Hz: 2H).

Mass IE m/z=379 M+. base peak

m/z=364 (M-CH3)+

m/z=203 C8H4NO2F3+.

m/z=92 C6H6N+

### Preparation of 5,5-dimethyl-3-(4-trifluoromethoxyphenyl)imidazolidine-2,4-dione

A solution of 7.08 g of 4-trifluoromethoxyaniline in 50 ml of toluene is added over 15 minutes to a suspension of 8.7 g of diphosgene and 1 g of plant charcoal in 100 ml of toluene, at a temperature in the region of −20° C. The mixture is stirred until the temperature is in the region of 20° C., and then refluxed for 3 hours. The mixture is cooled to a temperature in the region of 20° C. and then filtered through Celite, 5 g of methyl α-aminoisobutyrate hydrochloride, 50 ml of toluene and 10 ml of triethylamine are added to the filtrate. The mixture thus obtained is refluxed for 16 hours and then cooled to a temperature in the region of 20° C. The precipitate is filtered off and the filtrate is concentrated under reduced pressure, the residue obtained is purified by flash chromatography on a column packed with silica, conditioned and then eluted with a cyclohexane/ethyl acetate mixture (50/50, v/v). The fractions containing the expected product are concentrated under reduced pressure, and 3.4 g of 5,5-dimethyl-3-(4-trifluoromethoxyphenyl)imidazolidine-2,4-dione are thus obtained, the characteristics of which are as follows:

Mass IE m/z=288 M+. base peak

m/z=273 (M-CH3)+

m/z=203 C8H4NO2F3+.

1H NMR spectrum (300 MHz, (CD3)2SO d6, δ in ppm): 1.42 (s: 6H); 7.49 (d, J=9 Hz: 2H); 7.55 (d, J=9 Hz: 2H); 8.63 (unresolved peak: 1H).

### Example 1-64: 3-[4-(pentafluorothio)phenyl]-5,5-dimethyl-1-quinolin-4-ylmethylimidazolidine-2,4-dione trifluoroacetate

The compound is prepared from 0.16 mmol of resin, 0.48 mmol of Fmoc-AIB-(OH), 1.12 mmol of 4-quinolinecarboxaldehyde and 0.4 mmol of 4-(pentafluorothio)aniline, in the same manner as in Example 1-1. After purification by preparative LC-MS chromatography, 5 mg of the desired product are obtained.

EIMS ([M+H]⁺): 472

### Example 1-65: 3-[4-(pentafluorothio)phenyl]-5,5-dimethyl-1-pyrid-4-ylmethylimidazolidine-2,4-dione trifluoroacetate

The compound is prepared from 0.16 mmol of resin, 0.48 mmol of Fmoc-AIB-(OH), 1.12 mmol of 4-pyridinecarboxaldehyde and 0.4 mmol of 4-(pentafluorothio)aniline, in the same manner as in Example 1-1. After purification by preparative LC-MS chromatography, 13.2 mg of the desired product are obtained.

EIMS ([M+H]⁺): 422

### Example 1-66: 3-[4-(pentafluorothio)phenyl]-1-quinolin-4-ylmethylimidazolidine-2,4-dione trifluoroacetate

The compound is prepared from 0.1 mmol of resin, 0.3 mmol of N-Fmoc-Gly-(OH), 0.5 mmol of 4-quinolinecarboxaldehyde and 0.25 mmol of 4-(pentafluorothio)aniline, in the same manner as in Example 1-1. After purification by preparative LC-MS chromatography, 18 mg of the desired product are obtained.

EIMS ([M+H]⁺): 444

### Example 1-67: 3-[4-(pentafluorothio)phenyl]-1-pyrid-4-ylmethylimidazolidine-2,4-dione trifluoroacetate

The compound is prepared from 0.1 mmol of resin, 0.3 mmol of N-Fmoc-Gly-(OH), 0.5 mmol of 4-quinolinecarboxaldehyde and 0.25 mmol of 4-(pentafluorothio)aniline, in the same manner as in Example 1-1. After purification by preparative LC-MS chromatography, 18 mg of the desired product are obtained.

EIMS ([M+H]⁺): 394

### Example 1-68: 3-[4-(pentafluorothio)phenyl]-1-pyrid-2-yl-methylimidazolidine-2,4-dione trifluoroacetate

The compound is prepared from 0.1 mmol of resin, 0.3 mmol of N-Fmoc-Gly-(OH), 0.5 mmol of 4-quinolinecarboxaldehyde and 0.25 mmol of 4-(pentafluorothio)aniline, in the same manner as in Example 1-1. After purification by preparative LC-MS chromatography, 9 mg of the desired product are obtained.

EIMS ([M+H]⁺): 394

### Example 1-69: 3-[4-(pentafluorothio)phenyl]-1-pyrid-3-yl-methylimidazolidine-2,4-dione trifluoroacetate

The compound is prepared from 0.2 mmol of resin, 0.6 mmol of N-Fmoc-Gly-(OH), 1 mmol of 4-quinolinecarboxaldehyde and 0.5 mmol of 4-(pentafluorothio)aniline, in the same manner as in Example 1-1. After purification by preparative LC-MS chromatography, 42 mg of the desired product are obtained.

EIMS ([M+H]⁺): 394

The 6 reaction schemes below describe the preparation of compound of Formula (I) according to the present invention, especially among the products of Example 1-70 to Example 1-178 below.

Scheme 1 describes the preparation of hydantoin derivatives with amino substituents in the two positions of the pyridine ring (B²).

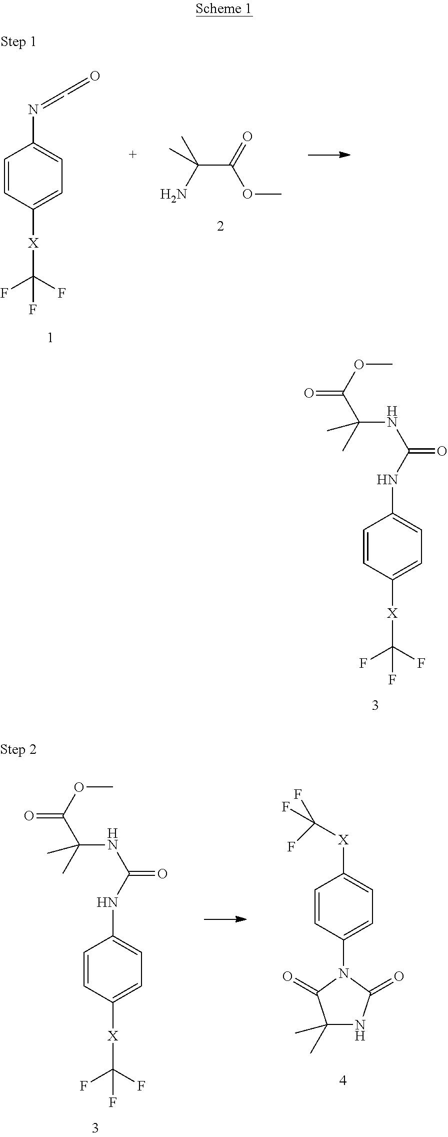

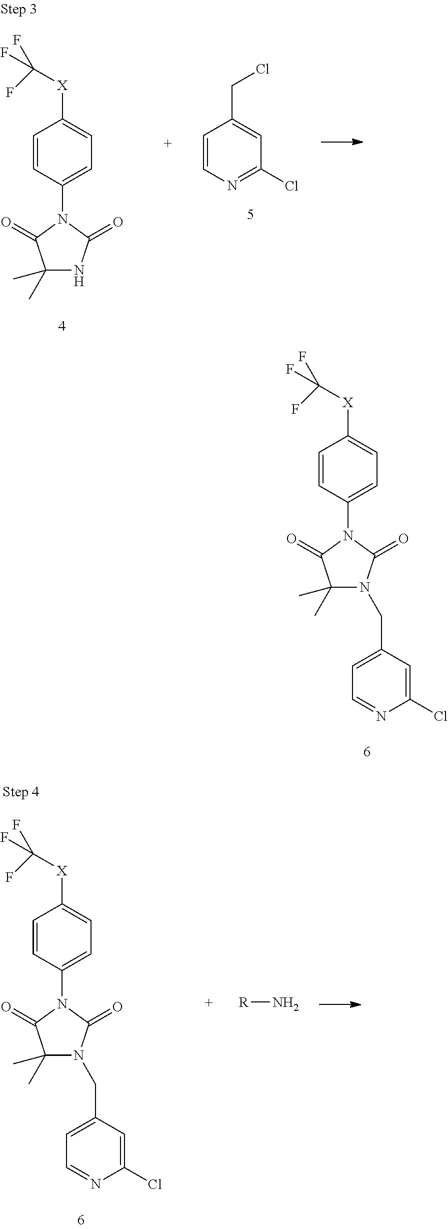

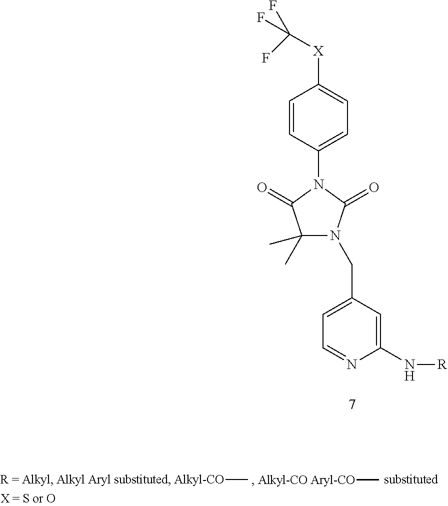

### Procedures for Scheme 1

### Step 1

(Syntheses Described for X=S, Analogous Scheme for X=O).

1-Isocyanato-4-trifluoromethylsulfanylbenzene (14.27 g, 65 mmol) is dissolved in 30 ml of dry CH₂Cl₂ and is cooled to 0° C. 7.5 g (65 mmol) of N-ethylmorpholine are added, followed by addition of 10 g (65 mmol) of methyl 2-amino-2-methylpropionate. The reaction mixture is left to reach 25° C. over a period of 6 hours, washed with water and, after removing the solvent, 21.8 g of methyl 2-methyl-2-[3-(4-trifluoro-methylsulfanylphenyl)ureido]propionate 3 are isolated.

### Step 2

30 g of 2-methyl-2-[3-(4-trifluoromethylsulfanylphenyl)ureido]propionate 3 are dissolved in a mixture of 225 ml of 3 N HCl and 230 ml of dioxane, refluxed for 6 hours and, after cooling to 4° C., the product 5,5-dimethyl-3-(4-trifluoromethylsulfanylphenyl)-imidazolidine-2,4-dione is isolated in the form of white crystals (24.14 g).

### Step 3

5,5-Dimethyl-3-(4-trifluoromethylsulfanylphenyl)imidazolidine-2,4-dione 3 (1 g, 3.29 mmol) is dissolved in 10 ml of DMF, 1.36 g of K₂CO₃ (3 eq, 9.87 mmol) and 909 mg of 2-chloro-4-chloromethylpyridine (3.95 mmol, 1.2 eq) are added, and the mixture is refluxed for 20 hours. The solvent is removed under vacuum and the residue is dissolved in CH₂Cl₂ and treated three times with active charcoal. After removing the solvent, the product 1-(2-chloropyrid-4-ylmethyl)-5,5-dimethyl-3-(4-trifluoromethylsulfanylphenyl)imidazolidine-2,4-dione is obtained (3.2 g; 56%).

### Analytical Data:

MS (LC-MS): 429.05; retention time: 2.49 min.

NMR: 1.4: s, 6H; 4.7: s, 2H; 7.45: m, 1H; 7.6: s, 1H; 7.7: d, 2H; 7.9: d, 2H; 8.4: m, 1H.

### Preparation of 2-chloro-4-chloromethylpyridine

10 g of 2-chloro-4-methylpyridine are dissolved in 30 ml of CH₃CN and a mixture of AIBN (3 g) and NCS (30 g) is added. The resulting mixture is refluxed for 4 hours. After removing the solvent, the crude product is further purified by distillation (boiling point: 70° C., 20 mtorr).

### Step 4

### General procedure for the palladium-catalyzed amination of the pyridine ring system in 1-(2-chloropyrid-4-ylmethyl)-5,5-dimethyl-3-(4-trifluoromethylsulfanyl-phenyl)imidazolidine-2,4-dione

100 mg (0.23 mmol) of 1-(2-chloropyrid-4-ylmethyl)-5,5-dimethyl-3-(4-trifluoro-methylsulfanylphenyl)imidazolidine-2,4-dione, 20 mg of Pd(OAc)₂, 60 mg of XANTPHOS and 300 mg of Cs₂CO₃ are transferred into a reaction tube with a screw stopper equipped with a rubber seal, and an argon atmosphere is generated in the tube. 1.5 equivalents (0.35 mmol) of the appropriate amine or amide are dissolved in 10 ml of toluene, the solution is transferred into the reaction tube mentioned above and the resulting mixture is heated at 95° C. for 6 to 10 hours depending on the reaction progress, monitored by LCMS.

After filtration, the solvent is removed under vacuum and the crude product is further purified by chromatography on an HPLC system.

Scheme 2 describes the preparation of urea and thiourea derivatives.

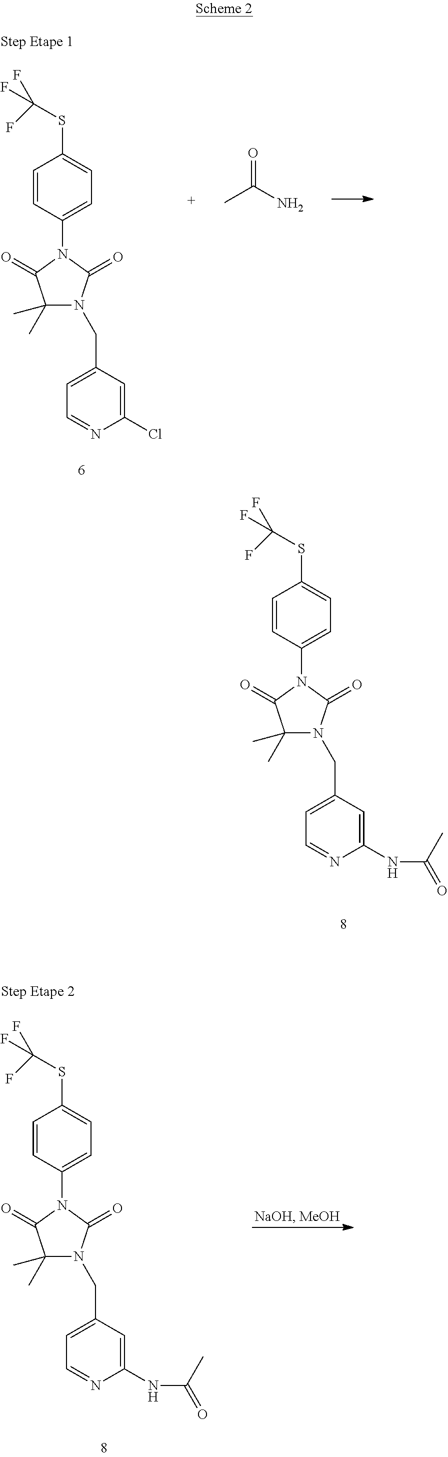

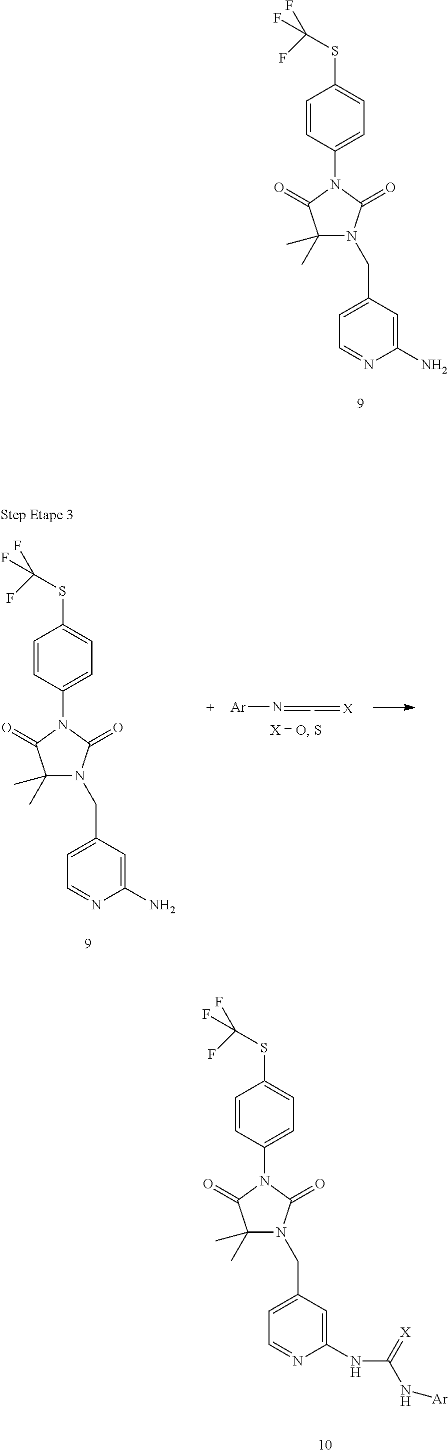

### Procedures for Scheme 2

### Step 1

This step is identical to step 4 of Scheme 1, with acetamide as reagent in the catalytic reaction.

Analytical data for N-{4-[5,5-dimethyl-2,4-dioxo-3-(4-trifluoromethylsulfanylphenyl) imidazolidin-1-ylmethyl]pyrid-2-yl}acetamide:

MS (LC-MS): 452.11; retention time: 1.82 min.

NMR: 1.4: s, 6H; 2.05: s, 3H; 4.65: s, 2H; 7.1: m, 1H; 7.65: d, 2H; 7.85: d, 2H; 8.15: m, 1H; 8.25: m, 1H.

### Step 2

500 mg (1.11 mmol) of N-{4-[5,5-dimethyl-2,4-dioxo-3-(4-trifluoro-methylsulfanylphenyl)imidazolidin-1-ylmethyl]pyrid-2-yl}acetamide are dissolved in MeOH, 1.5 mmol of NaOMe are added and the resulting mixture is refluxed for 4 hours. The solvent is removed, the residue is taken up in CH₂Cl₂ and washed twice with 10% NaHCO₃ solution and with water, and the organic phase is evaporated off. 340 mg (75%) of 1-(2-aminopyrid-4-ylmethyl)-5,5-dimethyl-3-(4-trifluoromethylsulfanylphenyl)imidazolidine-2,4-dione are isolated in this manner.

MS (LC-MS): 410.10; retention time: 1.57.

NMR: 1.4: s, 6H; 4.4: s, 2H; 6.35: s, 2H; 6.4: m, 1H; 6.5: m, 1H; 7.65: d, 2H; 7.9: m, 3H.

### Step 3

General procedure, all the urea and thiourea derivatives are prepared in this manner.

100 mg of 1-(2-aminopyrid-4-ylmethyl)-5,5-dimethyl-3-(4-trifluoro-methylsulfanylphenyl)imidazolidine-2,4-dione are dissolved in 5 ml of dioxane and 1.5 equivalents of the corresponding isocyanate or isothiocyanate are added. The reaction mixture is stirred at slightly elevated temperature up to the end, which is monitored by LCMS. The solvent is removed and further purification is performed by chromatography on an HPLC system.

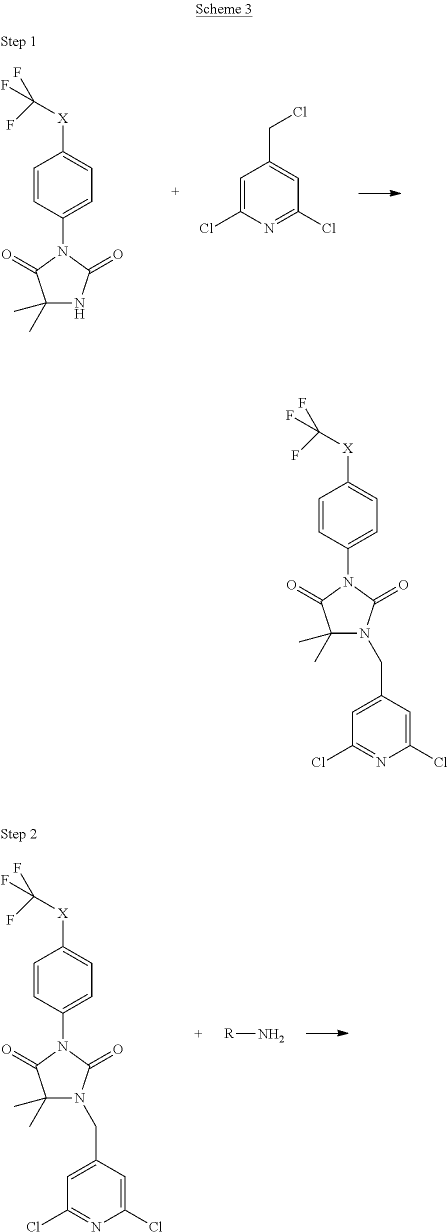

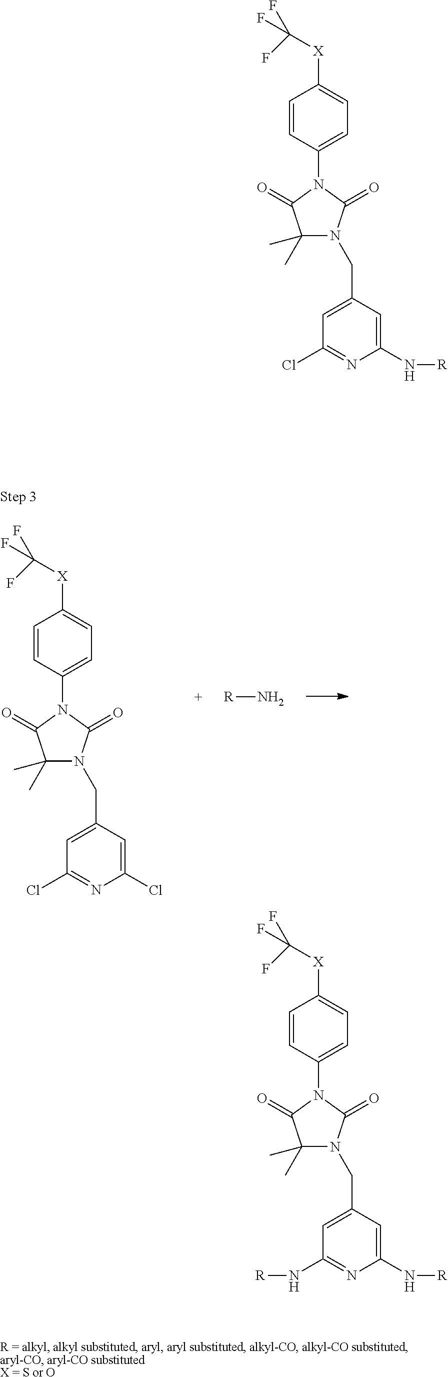

### Procedures for Scheme 3

### Step 1

(Syntheses Described for X=S, Analogous Scheme for X=O)

5,5-Dimethyl-3-(4-trifluoromethylsulfanylphenyl)imidazolidine-2,4-dione (1 000 mg/3.29 mmol) is dissolved in 20 ml of DMF, Cs₂CO₃ (3.21 g/9.9 mmol) and 2,6-dichloro-4-chloromethylpyridine (774 g/3.9 mmol) are added and the resulting mixture is heated at 80° C. for 6 hours. The solvent is removed and the residue is dissolved in CH₂Cl₂ and washed three times with water. After evaporating off the solvent, the crude material is further purified by chromatography on an HPLC system.

### Step 2

### General Procedure for the Pd-Catalyzed Monoamination of 1-(2,6-dichloropyrid-4-ylmethyl)-5, 5-dimethyl-3-(4-trifluoromethylsulfanylphenyl)imidazolidine-2,4-dione

This step is identical to Step 4 of Scheme 1, but only 1 equivalent of the corresponding amine or amide is used.

### Step 3

### General Procedure for the Pd-Catalyzed Bisamination of 1-(2,6-dichloropyrid-4-yl-methyl)-5, 5-dimethyl-3-(4-trifluoromethylsulfanylphenyl)imidazolidine-2,4-dione

This step is identical to Step 4 of Scheme 1, but in this case 2.2 equivalents of the corresponding amine or amide are used.

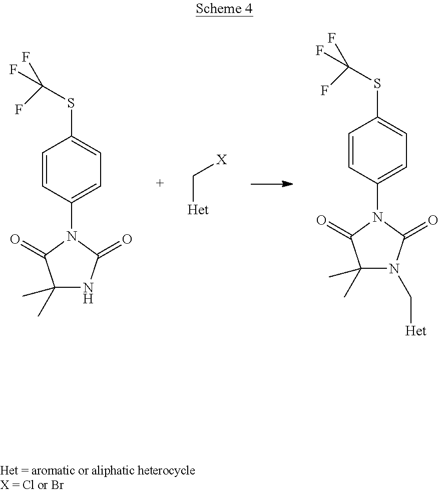

### Procedures for Scheme 4:

General procedure for the alkylation of 5,5-dimethyl-3-(4-trifluoro-methylsulfanylphenyl)imidazolidine-2,4-dione with aromatic or aliphatic heterocycles bearing a chloromethyl or bromomethyl substituent

5,5-Dimethyl-3-(4-trifluoromethylsulfanylphenyl)imidazolidine-2,4-dione (100 mg/0.33 mmol) is dissolved in 10 ml of DMF, Cs₂CO₃ (321 mg/0.99 mmol) and 0.49 mmol (1.5 equivalents) of the aromatic or aliphatic heterocycle substituted with a corresponding chloromethyl or bromomethyl group are added and the resulting mixture is heated at 80° C. for 6 hours. The solvent is removed and the crude material obtained is further purified by chromatography on an HPLC system.

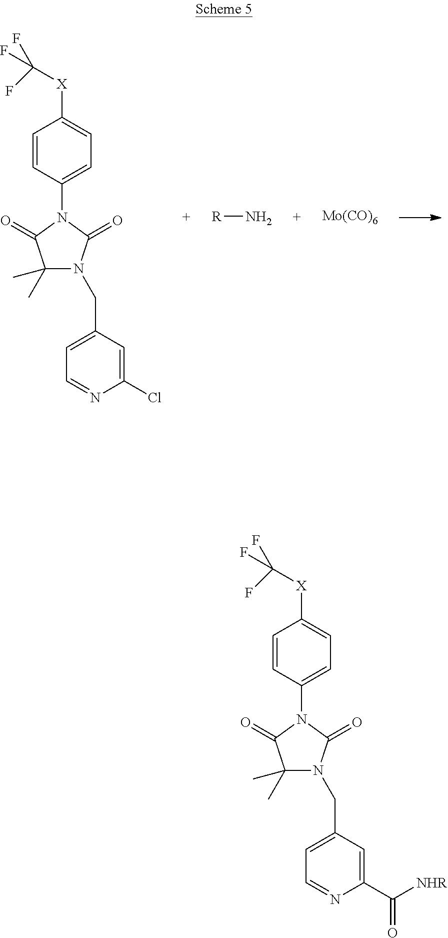

### Procedures for Scheme 5:

100 mg (0.23 mmol) of 1-(2-chloropyrid-4-ylmethyl)-5,5-dimethyl-3-(4-trifluoro-methyl sulfanylphenyl)imidazolidine-2,4-dione, 20 mg of Pd(OAc)₂, 60 mg of XANTPHOS, 300 mg of Cs₂CO₃ and 124 mg of Mo(CO)₆ (2 eq) are transferred into a reaction tube with a screw stopper, equipped with a rubber seal, and an argon atmosphere is generated in the tube.

1.5 equivalents (0.35 mmol) of the appropriate amine and 212 mg of DBU are dissolved in 10 ml of toluene, this solution is transferred into the reaction tube mentioned above and the resulting mixture is heated at 95° C. for 6 to 10 hours depending on the reaction progress, which is monitored by LCMS.

After filtration, the solvent is removed under vacuum and the crude product is further purified by chromatography on an HPLC system.

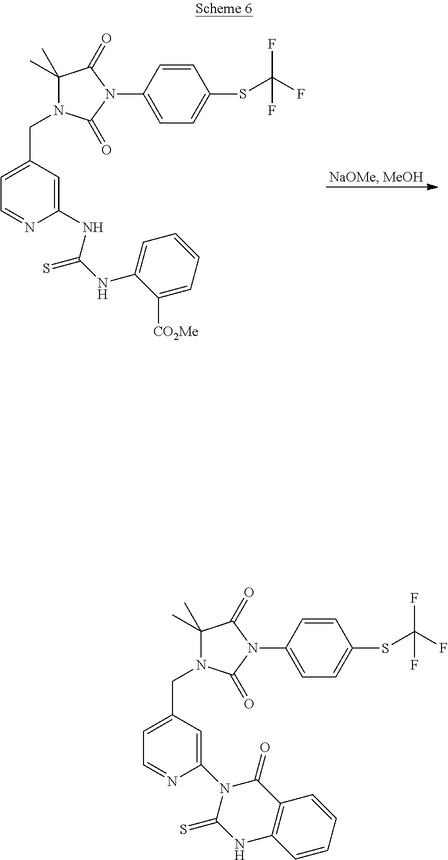 [SMILES: COC(=O)c1ccccc1NC(=S)Nc1cc(CN2C(=O)N(c3ccc(SC(F)(F)F)cc3)C(=O)C2(C)C)ccn1 ; CC1(C)C(=O)N(c2ccc(SC(F)(F)F)cc2)C(=O)N1Cc1ccnc(-n2c(=S)[nH]c3ccccc3c2=O)c1]

### Procedures for Scheme 6:

30 mg (0.05 mmol) of methyl 2-(3-{4-[5,5-dimethyl-2,4-dioxo-3-(4-trifluoro-methylsulfanylphenyl)imidazolidin-1-ylmethyl]pyrid-2-yl}thioureido)benzoate are dissolved in 2 ml of MeOH, 0.1 mmol of NaOMe is added and the resulting mixture is stirred overnight at room temperature. After evaporating off the solvent, the crude 5,5-dimethyl-1-[2-(4-oxo-2-thioxo-1,4-dihydro-2H-quinazolin-3-yl)pyrid-4-yl-methyl]-3-(4-trifluoromethylsulfanylphenyl)imidazolidine-2,4-dione is further purified by chromatography on an HPLC system.

### Example 1-70: N-{4-[5,5-dimethyl-2,4-dioxo-3-(4-trifluoro-methylsulfanylphenyl)imidazolidin-1-ylmethyl]pyrid-2-yl}propionamide; compound with trifluoroacetic acid

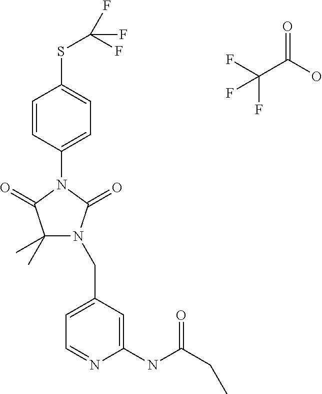 [SMILES: CCC(=O)Nc1cc(CN2C(=O)N(c3ccc(SC(F)(F)F)cc3)C(=O)C2(C)C)ccn1 ; O=C(O)C(F)(F)F]

MS (LC-MS): 466.13, Retention time: 1.83 min.

NMR: 1.05: t, 3H; 1.40: s, 6H; 2.35: q, 2H; 4.65: s, 2H; 7.1: d, 1H; 7.65: d, 2H; 7.85: d, 2H; 8.15: s, 1H; 8.25: d, 1H.

The synthesis is described in Scheme 1.

### Example 1-71: N-{4-[5,5-dimethyl-2,4-dioxo-3-(4-trifluoro-methylsulfanylphenyl)imidazolidin-1-ylmethyl]pyrid-2-yl}isobutyramide; compound with trifluoroacetic acid

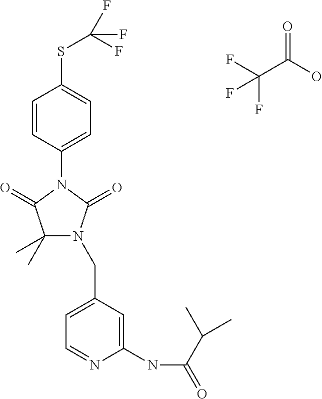 [SMILES: CC(C)C(=O)Nc1cc(CN2C(=O)N(c3ccc(SC(F)(F)F)cc3)C(=O)C2(C)C)ccn1 ; O=C(O)C(F)(F)F]

MS (LC-MS): 480.14, Retention time: 1.93 min.

NMR: 1.10: d, 6H; 1.40: s, 6H; 2.75: s, 1H; 4.65: s, 2H; 7.15: d, 1H; 7.65: d, 2H; 7.85: d, 2H; 8.15: s, 1H; 8.25: d, 1H.

The synthesis is described in Scheme 1.

### Example 1-72: N-{4-[5,5-dimethyl-2,4-dioxo-3-(4-trifluoromethyl-sulfanylphenyl)imidazolidin-1-ylmethyl]pyrid-2-yl}-3-morpholin-4-ylpropionamide; compound with trifluoroacetic acid

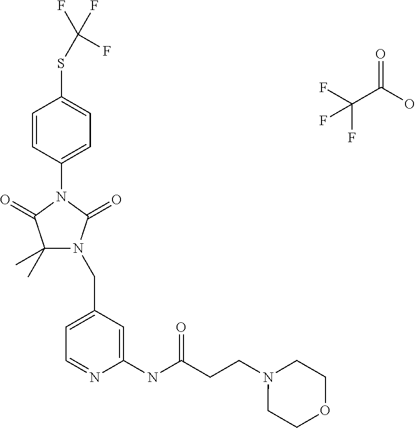 [SMILES: CC1(C)C(=O)N(c2ccc(SC(F)(F)F)cc2)C(=O)N1Cc1ccnc(NC(=O)CCN2CCOCC2)c1 ; O=C(O)C(F)(F)F]

MS (LC-MS): 551.18, Retention time: 1.54 min.

NMR: 1.40: s, 6H; 2.85: m, 2H; 3.1: m, 2H; 3.4: m, 2H; 3.95: m, 2H; 4.65: s, 2H; 7.15: d, 1H; 7.65: d, 2H; 7.85: d, 2H; 8.15: s, 1H; 8.25: d, 1H.

The synthesis is described in Scheme 1.

### Example 1-73: N-{4-[5,5-dimethyl-2,4-dioxo-3-(4-trifluoro-methylsulfanylphenyl)imidazolidin-1-ylmethyl]pyrid-2-yl}-3-[4-(2-hydroxyethyl)piperazin-1-yl]propionamide

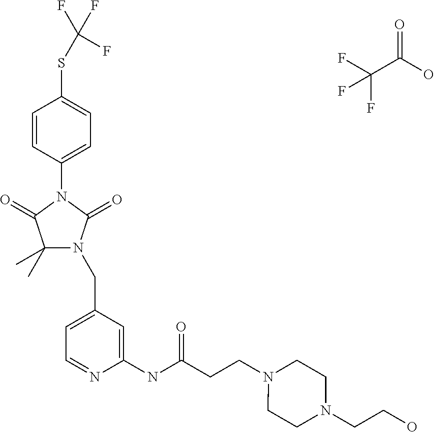 [SMILES: CC1(C)C(=O)N(c2ccc(SC(F)(F)F)cc2)C(=O)N1Cc1ccnc(NC(=O)CCN2CCN(CCO)CC2)c1 ; O=C(O)C(F)(F)F]

MS (LC-MS): 594.22, Retention time: 1.40 min.

NMR: 1.40: s, 6H; 2.55 to 3.50: m, 12H; 4.65: s, 2H; 7.15: m, 1H; 7.65: d, 2H; 7.85: d, 2H; 8.15: s, 1H; 8.25: d, 1H.

The synthesis is described in Scheme 1.

### Example 1-74: N-{4-[5,5-dimethyl-2,4-dioxo-3-(4-trifluoro-methylsulfanylphenyl)imidazolidin-1-ylmethyl]pyrid-2-yl}-3-(4-methylpiperazin-1-yl)propionamide

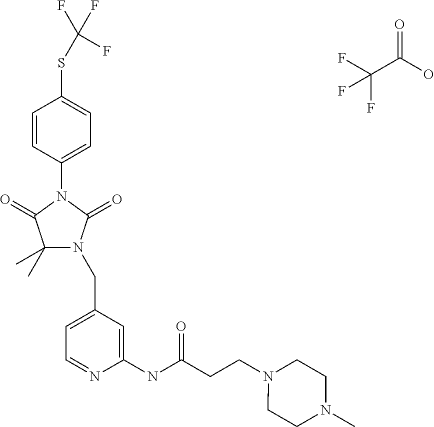 [SMILES: CN1CCN(CCC(=O)Nc2cc(CN3C(=O)N(c4ccc(SC(F)(F)F)cc4)C(=O)C3(C)C)ccn2)CC1 ; O=C(O)C(F)(F)F]

MS (LC-MS): 564.21, Retention time: 1.41 min.

NMR: 1.40: s, 6H; 2.55 to 3.50: m, 12H; 2.75: s, 3H; 4.65: s, 2H; 7.15: m, 1H; 7.65: d, 2H; 7.85: d, 2H; 8.15: s, 1H; 8.25: d, 1H.

The synthesis is described in Scheme 1.

### Example 1-75: {4-[5,5-dimethyl-2,4-dioxo-3-(4-trifluoromethyl-sulfanylphenyl)imidazolidin-1-ylmethyl]pyrid-2-yl}-cyclopropanecarboxamide

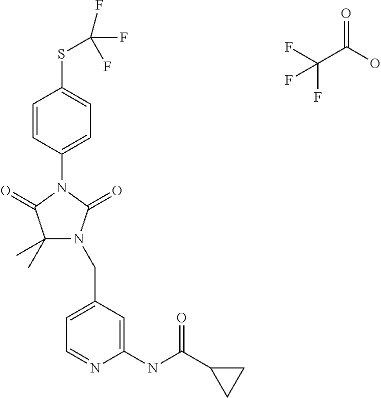 [SMILES: CC1(C)C(=O)N(c2ccc(SC(F)(F)F)cc2)C(=O)N1Cc1ccnc(NC(=O)C2CC2)c1 ; O=C(O)C(F)(F)F]

MS (LC-MS): 478.13, Retention time: 1.99 min.

NMR: 0.80: m, 4H; 1.15: t, 1H; 1.40: s, 6H; 4.65: s, 2H; 7.15: m, 1H; 7.65: d, 2H; 7.85: d, 2H; 8.15: s, 1H; 8.25: d, 1H.

The synthesis is described in Scheme 1.

### Example 1-76: 5,5-dimethyl-1-[2-(pyrid-2-ylamino)pyrid-4-ylmethyl]-3-(4-trifluoromethylsulfanylphenyl)imidazolidine-2,4-dione

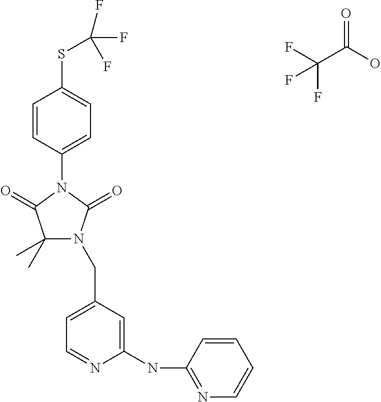 [SMILES: CC1(C)C(=O)N(c2ccc(SC(F)(F)F)cc2)C(=O)N1Cc1ccnc(Nc2ccccn2)c1 ; O=C(O)C(F)(F)F]

MS (LC-MS): 487.13, Retention time: 1.85 min.

NMR: 1.40: s, 6H; 4.65: s, 2H; 7.3: m, 4H; 7.65: d, 2H; 7.85: d, 2H; 8.05: s, 1H; 8.35: m, 1H.

The synthesis is described in Scheme 1.

### Example 1-77: N-{4-[5,5-dimethyl-2,4-dioxo-3-(4-trifluoro-methylsulfanylphenyl)imidazolidin-1-ylmethyl]pyrid-2-yl}-3-pyrrolidin-1-ylpropionamide

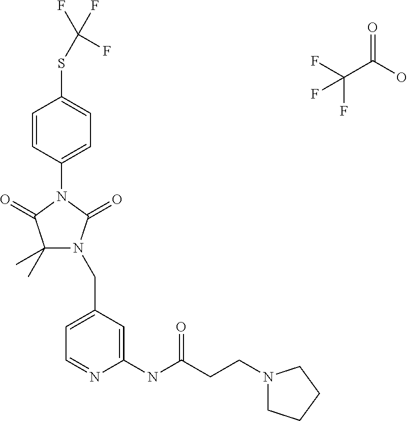 [SMILES: CC1(C)C(=O)N(c2ccc(SC(F)(F)F)cc2)C(=O)N1Cc1ccnc(NC(=O)CCN2CCCC2)c1 ; O=C(O)C(F)(F)F]

MS (LC-MS): 535.19, Retention time: 1.52 min.

NMR: 1.40: s, 6H; 1.8: m, 2H; 2.0: m, 2H; 2.85: m, 2H; 3.0: m, 2H; 3.4: m, 2H; 3.5: m, 2H; 4.65: s, 2H; 7.2: m, 1H; 7.65: d, 2H; 7.85: d, 2H; 8.15: s, 1H; 8.40: m, 1H.

The synthesis is described in Scheme 1.

### Example 1-78: 5,5-dimethyl-1-{2-[3-(4-methylpiperazin-1-yl)propylamino]pyrid-4-ylmethyl}-3-(4-trifluoromethylsulfanylphenyl)imidazolidine-2,4-dione

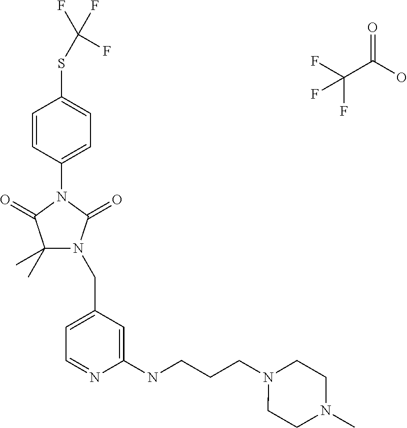 [SMILES: CN1CCN(CCCNc2cc(CN3C(=O)N(c4ccc(SC(F)(F)F)cc4)C(=O)C3(C)C)ccn2)CC1 ; O=C(O)C(F)(F)F]

MS (LC-MS): 550.23, Retention time: 1.52 min.

NMR: 1.40: s, 6H; 2.6 to 3.5: m, 14H; 2.75: s, 3H; 4.65: s, 2H; 6.85: m, 1H; 6.95: m, 1H; 7.65: d, 2H; 7.9: m, 3H.

The synthesis is described in Scheme 1.

### Example 1-79: 5,5-dimethyl-1-{2-[3-(4-ethylpiperazin-1-yl)propylamino]pyrid-4-ylmethyl}-3-(4-trifluoromethylsulfanylphenyl)imidazolidine-2,4-dione

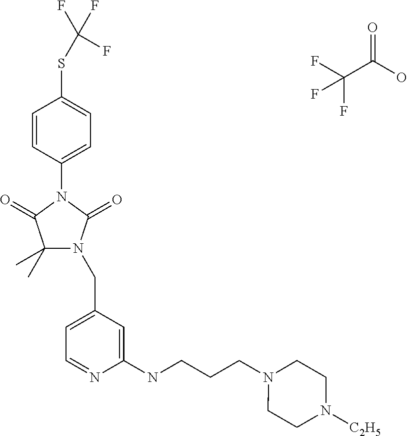 [SMILES: CCN1CCN(CCCNc2cc(CN3C(=O)N(c4ccc(SC(F)(F)F)cc4)C(=O)C3(C)C)ccn2)CC1 ; O=C(O)C(F)(F)F]

The synthesis is described in Scheme 1.

### Example 1-80: 1-[2-(3-methoxyphenylamino)pyrid-4-ylmethyl]-5,5-dimethyl-3-(4-trifluoromethylsulfanylphenyl)imidazolidine-2,4-dione

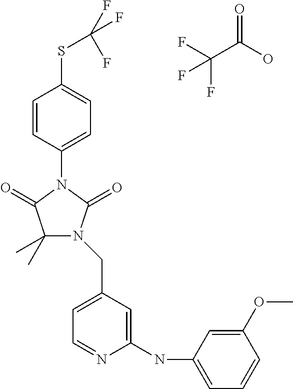 [SMILES: COc1cccc(Nc2cc(CN3C(=O)N(c4ccc(SC(F)(F)F)cc4)C(=O)C3(C)C)ccn2)c1 ; O=C(O)C(F)(F)F]

MS (LC-MS): 516.14, Retention time: 1.90 min.

NMR: 1.40: s, 6H; 3.7: s, 3H; 4.65: s, 2H; 6.6: m, 1H; 6.9: m, 2H; 7.15: m, 1H; 7.2: m, 1H; 7.25: m, 1H; 7.65: d, 2H; 7.85: d, 2H; 8.05: s, 1H.

The synthesis is described in Scheme 1.

### Example 1-81: N-{4-[5,5-dimethyl-2,4-dioxo-3-(4-trifluoromethyl-sulfanylphenyl)imidazolidin-1-ylmethyl]pyrid-2-yl}-3-(4-ethylpiperazin-1-yl)propionamide

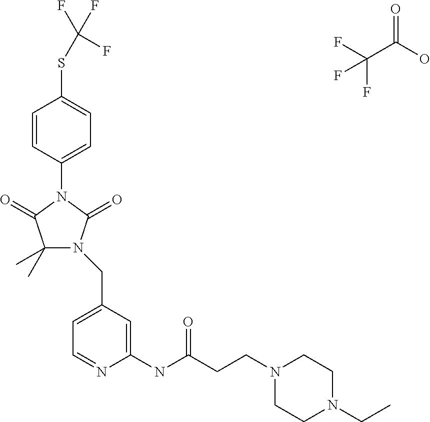 [SMILES: CCN1CCN(CCC(=O)Nc2cc(CN3C(=O)N(c4ccc(SC(F)(F)F)cc4)C(=O)C3(C)C)ccn2)CC1 ; O=C(O)C(F)(F)F]

MS (LC-MS): 516.14, Retention time: 1.90 min.

NMR: 1.15: t, 3H; 1.40: s, 6H; 2.5 to 3.7: m, 14H; 4.65: s, 2H; 7.15: m, 1H; 7.65: d, 2H; 7.85: d, 2H; 8.15: s, 1H; 8.25: m, 1H.

The synthesis is described in Scheme 1.

### Example 1-82: 5,5-dimethyl-1-[2-(3-methyl-2-oxopyrrolidin-1-yl)pyrid-4-ylmethyl]-3-(4-trifluoromethylsulfanylphenyl)imidazolidine-2,4-dione; compound with trifluoroacetic acid

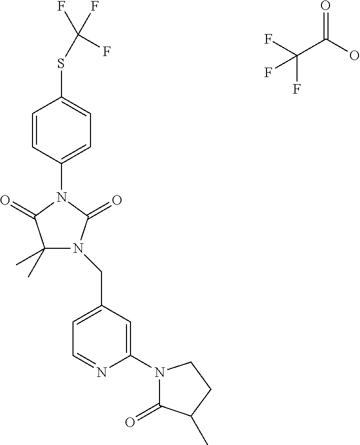 [SMILES: CC1CCN(c2cc(CN3C(=O)N(c4ccc(SC(F)(F)F)cc4)C(=O)C3(C)C)ccn2)C1=O ; O=C(O)C(F)(F)F]

MS (LC-MS): 492.14; Retention time: 2.23 min.

NMR: 1.15: d, 3H; 1.40: s, 6H; 1.60: m, 1H; 2.30: m, 1H; 2.70: m, 1H; 3.80: m, 1H; 4.0: m, 1H; 4.65: s, 2H; 7.15: m, 1H; 7.65: d, 2H; 7.85: d, 2H; 8.35: m, 2H.

The synthesis is described in Scheme 1.

### Example 1-83: 5,5-dimethyl-1-((2-(3-methyl-2-oxopyrrolidin-1-yl)pyridin-4-yl)methyl)-3-(4-((trifluoromethyl)thio)phenyl)imidazolidine-2,4-dione with trifluoracetic acid

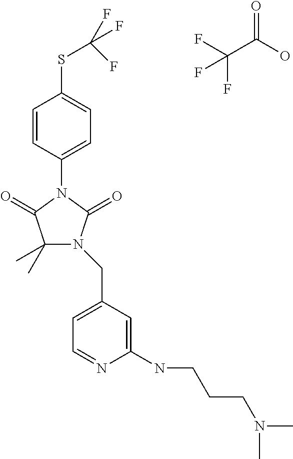 [SMILES: CN(C)CCCNc1cc(CN2C(=O)N(c3ccc(SC(F)(F)F)cc3)C(=O)C2(C)C)ccn1 ; O=C(O)C(F)(F)F]

MS (LC-MS): 495.19; Retention time: 1.43 min.

The synthesis is described in Scheme 1.

### Example 1-84: 5,5-dimethyl-1-[2-(4-pyrid-2-ylpiperazin-1-yl)pyrid-4-ylmethyl]-3-(4-trifluoromethylsulfanylphenyl)imidazolidine-2,4-dione; compound with trifluoroacetic acid

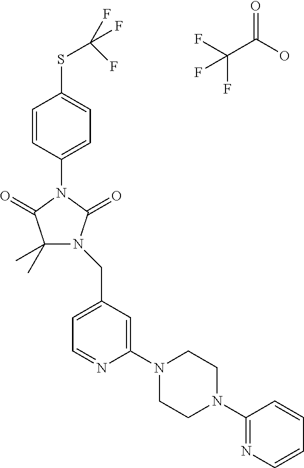 [SMILES: CC1(C)C(=O)N(c2ccc(SC(F)(F)F)cc2)C(=O)N1Cc1ccnc(N2CCN(c3ccccn3)CC2)c1 ; O=C(O)C(F)(F)F]

MS (LC-MS): 556.19; Retention time: 1.56 min.

The synthesis is described in Scheme 1.

### Example 1-85: N-{4-[5,5-dimethyl-2,4-dioxo-3-(4-trifluoro-methylsulfanylphenyl)imidazolidin-1-ylmethyl]pyrid-2-yl}-3-piperid-1-ylpropionamide; compound with trifluoroacetic acid

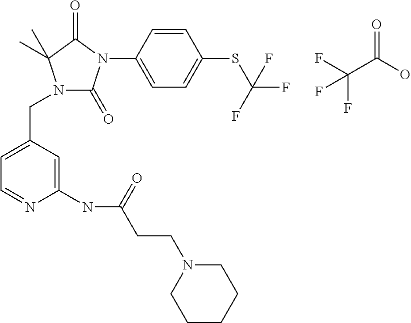

MS (LC-MS): 549.22; Retention time: 1.62 min.

NMR: 1.45: s, 6H; 1.55: m, 3H; 1.80: m, 2H; 2.80: m, 4H; 3.3 to 3.5: m, 7H; 4.65: s, 2H; 7.15: m, 1H; 7.65: d, 2H; 7.85: m, 2H; 8.15: m, 1H; 8.3: m, 1H; 9.0: m, 1H.

The synthesis is described in Scheme 1.

### Example 1-86: 1-[2-(3-imidazol-1-ylpropylamino)pyrid-4-ylmethyl]-5,5-dimethyl-3-(4-trifluoromethylsulfanylphenyl)imidazolidine-2,4-dione; compound with trifluoroacetic acid

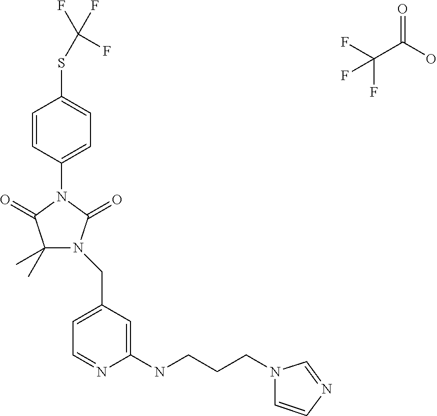 [SMILES: CC1(C)C(=O)N(c2ccc(SC(F)(F)F)cc2)C(=O)N1Cc1ccnc(NCCCn2ccnc2)c1 ; O=C(O)C(F)(F)F]

MS (LC-MS): 518.17; Retention time: 1.57 min.

The synthesis is described in Scheme 1.

### Example 1-87: 1-[2-(4-ethylpyrid-2-ylamino)pyrid-4-yl-methyl]-5,5-dimethyl-3-(4-trifluoromethylsulfanylphenyl)imidazolidine-2,4-dione

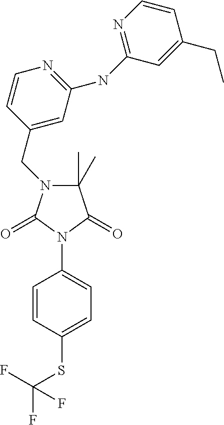 [SMILES: CCc1ccnc(Nc2cc(CN3C(=O)N(c4ccc(SC(F)(F)F)cc4)C(=O)C3(C)C)ccn2)c1]

MS (LC-MS): 515.16; Retention time: 1.85 min.

NMR: 1.15: t, 3H; 1.45: s, 6H; 2.7: q, 2H; 4.75: s, 2H; 7.1: broad s, 2H; 7.3: broad s 2HM; 7.65: d, 2H; 7.85: d, 2H; 8.25: s, 1H; 8.45: s, 1H.

The synthesis is described in Scheme 1.

### Example 1-88: 1-[2-(6-ethylpyrid-2-ylamino)pyrid-4-ylmethyl]-5,5-dimethyl-3-(4-trifluoromethylsulfanylphenyl)imidazolidine-2,4-dione

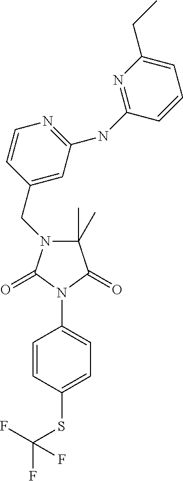 [SMILES: CCc1cccc(Nc2cc(CN3C(=O)N(c4ccc(SC(F)(F)F)cc4)C(=O)C3(C)C)ccn2)n1]

MS (LC-MS): 515.16; Retention time: 1.95 min.

NMR: 1.15: t, 3H; 1.45: s, 6H; 2.7: q, 2H; 4.75: s, 2H; 7.1: broad s, 2H; 7.3: broad s 2HM; 7.65: d, 2H; 7.85: d, 2H; 8.25: s, 1H; 8.45: s, 1H.

The synthesis is described in Scheme 1.

### Example 1-89: 5,5-dimethyl-1-[2-(quinolin-2-ylamino)pyrid-4-ylmethyl]-3-(4-trifluoromethylsulfanylphenyl)imidazolidine-2,4-dione

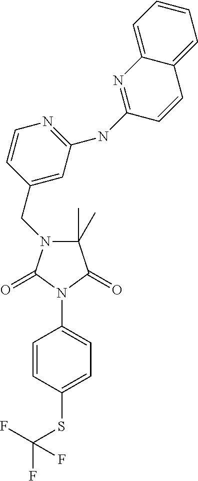 [SMILES: CC1(C)C(=O)N(c2ccc(SC(F)(F)F)cc2)C(=O)N1Cc1ccnc(Nc2ccc3ccccc3n2)c1]

MS (LC-MS): 537.14; Retention time: 1.95 min.

NMR: 1.45: s, 6H; 4.75: s, 2H; 7.4 to 7.6: m, 6H; 7.65: d, 2H; 7.85: d, 2H; 8.00: broad s, 1H; 8.50: broad s, 2H.

The synthesis is described in Scheme 1.

### Example 1-90: 5,5-dimethyl-1-[2-(4-methylpyrid-2-ylamino)pyrid-4-ylmethyl]-3-(4-trifluoromethylsulfanylphenyl)imidazolidine-2,4-dione

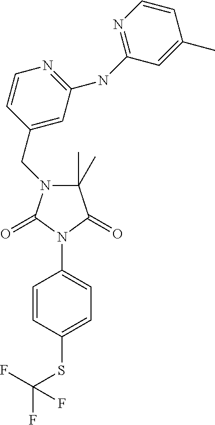 [SMILES: Cc1ccnc(Nc2cc(CN3C(=O)N(c4ccc(SC(F)(F)F)cc4)C(=O)C3(C)C)ccn2)c1]

MS (LC-MS): 501.14; Retention time: 1.80 min.

NMR: 1.45: s, 6H; 2.4: s, 3H; 4.75: s, 2H; 7.1: m, 2H; 7.25: m, 2H; 7.65: d, 2H; 7.85: d, 2H; 8.20: broad s, 1H; 8.45: broad s, 1H.

The synthesis is described in Scheme 1.

### Example 1-91: 5,5-dimethyl-1-[2-(6-methylpyrid-2-ylamino)pyrid-4-ylmethyl]-3-(4-trifluoromethylsulfanylphenyl)imidazolidine-2,4-dione

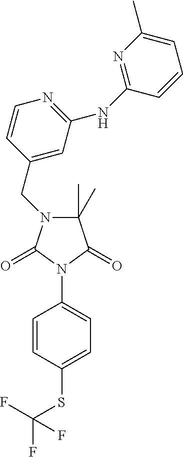 [SMILES: Cc1cccc(Nc2cc(CN3C(=O)N(c4ccc(SC(F)(F)F)cc4)C(=O)C3(C)C)ccn2)n1]

MS (LC-MS): 501.14; Retention time: 1.85 min.

NMR: 1.45: s, 6H; 2.55: s, 3H; 4.75: s, 2H; 7.1: m, 1H; 7.25: m, 2H; 7.4: s, 1H; 7.65: d, 2H; 7.85: m, 3H; 8.45: broad s, 1H.

The synthesis is described in Scheme 1.

### Example 1-92: 1-[2-(3,5-dichloropyrid-2-ylamino)pyrid-4-ylmethyl]-5,5-dimethyl-3-(4-trifluoromethylsulfanylphenyl)imidazolidine-2,4-dione

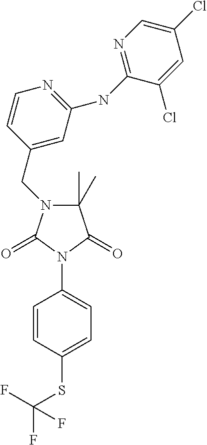 [SMILES: CC1(C)C(=O)N(c2ccc(SC(F)(F)F)cc2)C(=O)N1Cc1ccnc(Nc2ncc(Cl)cc2Cl)c1]

MS (LC-MS): 555.05; Retention time: 1.96 min.

NMR: 1.45: s, 6H; 4.70: s, 2H; 7.1: m, 1H; 7.20: m, 1H; 7.65: d, 2H; 7.85: m, 3H; 8.30: m, 3H.

The synthesis is described in Scheme 1.

### Example 1-93: 1-[2-(4,6-dimethylpyrid-2-ylamino)pyrid-4-ylmethyl]-5,5-dimethyl-3-(4-trifluoromethylsulfanylphenyl)imidazolidine-2,4-dione

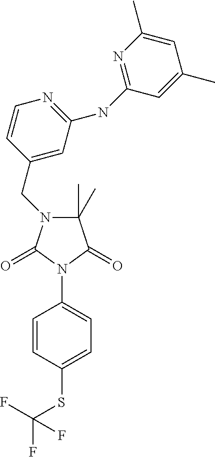 [SMILES: Cc1cc(C)nc(Nc2cc(CN3C(=O)N(c4ccc(SC(F)(F)F)cc4)C(=O)C3(C)C)ccn2)c1]

MS (LC-MS): 515.16; Retention time: 2.00 min.

NMR: 1.45: s; 2.35: s, 3H; 2.6: s, 3H; 6H; 4.75: s, 2H; 7.0: m, 2H; 7.35: m, 2H; 7.70: d, 2H; 7.9: d, 2H; 8.40: broad s, 1H.

The synthesis is described in Scheme 1.

### Example 1-94: 5,5-dimethyl-1-[2-(methylpyrid-2-ylamino)pyrid-4-ylmethyl]-3-(4-trifluoromethylsulfanylphenyl)imidazolidine-2,4-dione

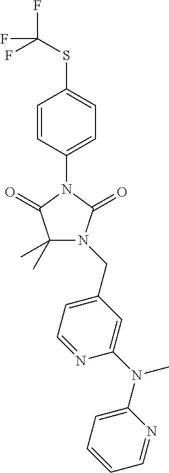 [SMILES: CN(c1ccccn1)c1cc(CN2C(=O)N(c3ccc(SC(F)(F)F)cc3)C(=O)C2(C)C)ccn1]

MS (LC-MS): 501.14; Retention time: 1.74 min.

The synthesis is described in Scheme 1.

### Example 1-95: 5,5-dimethyl-1-[2-(pyrid-4-ylamino)pyrid-4-ylmethyl]-3-(4-trifluoromethylsulfanylphenyl)imidazolidine-2,4-dione

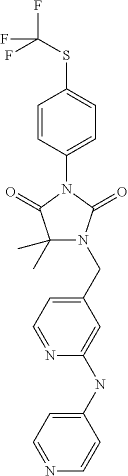 [SMILES: CC1(C)C(=O)N(c2ccc(SC(F)(F)F)cc2)C(=O)N1Cc1ccnc(Nc2ccncc2)c1]

MS (LC-MS): 487.13; Retention time: 1.69 min.

NMR: 1.45: s; 4.70: s, 2H; 7.15: m, 1H; 7.25: m, 1H; 7.65: d, 2H; 7.9: d, 2H; 8.10: broad s, 2H; 8.40: s, 1H; 8.50: m, 2H.

The synthesis is described in Scheme 1.

### Example 1-96: 5,5-dimethyl-1-[2-(pyrid-3-ylamino)pyrid-4-ylmethyl]-3-(4-trifluoromethylsulfanylphenyl)imidazolidine-2,4-dione

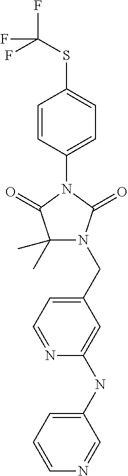 [SMILES: CC1(C)C(=O)N(c2ccc(SC(F)(F)F)cc2)C(=O)N1Cc1ccnc(Nc2cccnc2)c1]

MS (LC-MS): 487.13; Retention time: 1.69 min.

NMR: 1.45: s; 4.60: s, 2H; 6.95: s, 1H; 7.05: s, 1H; 7.7: d, 2H; 7.8: m, 1H; 7.85: d, 2H; 8.25: s, 1H; 8.35: s, 1H; 8.45: m, 1H; 9.4: s, 1H.

The synthesis is described in Scheme 1.

### Example 1-97: N-{4-[5,5-dimethyl-2,4-dioxo-3-(4-trifluoro-methylsulfanylphenyl)imidazolidin-1-ylmethyl]pyrid-2-yl}-3-(2-oxoazepan-1-yl)propionamide

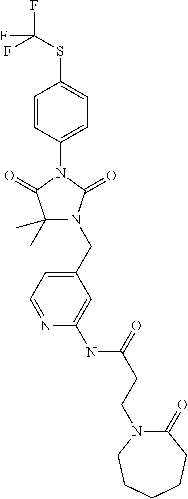 [SMILES: CC1(C)C(=O)N(c2ccc(SC(F)(F)F)cc2)C(=O)N1Cc1ccnc(NC(=O)CCN2CCCCCC2=O)c1]

MS (LC-MS): 577.20; Retention time: 2.00 min.

NMR: 1.45: s, 6H; 1.5: m, 4H; 1.55: m, 2H; 2.35: m, 2H; 2.6: m, 4H; 3.3 m, 2H; 4.65: s, 2H; 7.15: s, 1H; 7.65: d, 2H; 7.85: d, 2H; 8.1: s, 1H; 8.35: s, 1H.

The synthesis is described in Scheme 1.

### Example 1-98: 3-(benzylmethylamino)-N-{4-[5,5-dimethyl-2,4-dioxo-3-(4-trifluoromethylsulfanylphenyl)imidazolidin-1-ylmethyl]pyrid-2-yl}propionamide

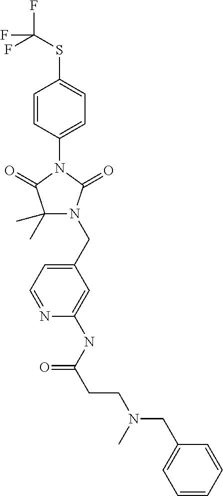 [SMILES: CN(CCC(=O)Nc1cc(CN2C(=O)N(c3ccc(SC(F)(F)F)cc3)C(=O)C2(C)C)ccn1)Cc1ccccc1]

MS (LC-MS): 585.20, Retention time: 1.73 min.

NMR: 1.45: s, 6H; 2.2: s, 3H; 2.95: m, 2H; 3.3: m, 1H; 4.3: m, 1H; 4.4: m, 1H; 4.65: s, 2H; 7.2: s, 1H; 7.45: m, 2H; 7.50: m, 2H; 7.65: d, 2H; 7.85: d, 2H; 8.1: m, 1H; 8.3: m, 1H.

The synthesis is described in Scheme 1.

### Example 1-99: N-{4-[5,5-dimethyl-2,4-dioxo-3-(4-trifluoro-methylsulfanylphenyl)imidazolidin-1-ylmethyl]pyrid-2-yl}-2-pyrrolidin-1-ylacetamide

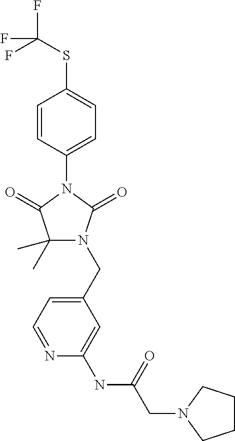 [SMILES: CC1(C)C(=O)N(c2ccc(SC(F)(F)F)cc2)C(=O)N1Cc1ccnc(NC(=O)CN2CCCC2)c1]

MS (LC-MS): 521.17, Retention time: 1.68 min.

NMR: 1.45: s, 6H; 1.85: m, 2H; 2.00: m, 2H; 3.1: m, 2H; 4.25: m, 2H; 4.7: s, 2H; 7.25: m, 1H; 7.65: d, 2H; 7.85: d, 2H; 8.1: broad s, 1H; 8.3: s, 1H.

The synthesis is described in Scheme 1.

### Example 1-100: N-{4-[5,5-dimethyl-2,4-dioxo-3-(4-trifluoro-methylsulfanylphenyl)imidazolidin-1-ylmethyl]pyrid-2-yl}-2-(4-pyrid-2-ylpiperazin-1-yl)acetamide

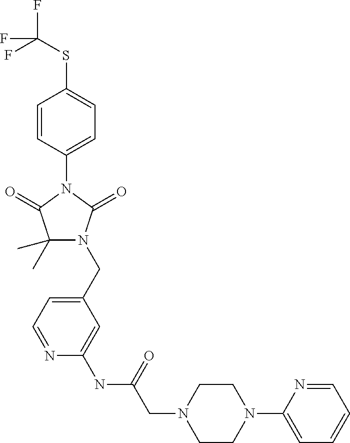 [SMILES: CC1(C)C(=O)N(c2ccc(SC(F)(F)F)cc2)C(=O)N1Cc1ccnc(NC(=O)CN2CCN(c3ccccn3)CC2)c1]

MS (LC-MS): 613.21, Retention time: 1.60 min.

NMR: 1.45: s, 6H; 3.3: broad m, 4H; 4.35: broad m, 4H; 4.65: s, 2H; 6.7: m, 1H; 6.9: m, 1H; 7.25: s, 1H; 7.65, m+d: 3H; 7.85: d, 2H; 8.15: m, 2H; 8.35: s, 1H.

The synthesis is described in Scheme 1.

### Example 1-101: 2-[(2-dimethylaminoethyl)methylamino]-N-{4-[5,5-dimethyl-2,4-dioxo-3-(4-trifluoromethylsulfanylphenyl)imidazolidin-1-ylmethyl]pyrid-2-yl}acetamide

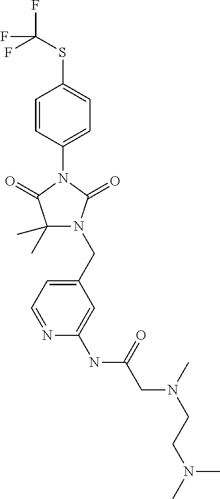 [SMILES: CN(C)CCN(C)CC(=O)Nc1cc(CN2C(=O)N(c3ccc(SC(F)(F)F)cc3)C(=O)C2(C)C)ccn1]

MS (LC-MS): 552.21, Retention time: 1.52 min.

NMR: 1.45: s, 6H; 2.75: s, 6H; 4.65: s, 2H; 7.20: m, 1H; 7.65: d, 2H; 7.85: d, 2H; 8.15: s, 1H; 8.30: m, 1H.

The synthesis is described in Scheme 1.

### Example 1-102: 3-[(2-dimethylaminoethyl)methylamino]-N-{4-[5,5-dimethyl-2,4-dioxo-3-(4-trifluoromethylsulfanylphenyl)imidazolidin-1-ylmethyl]pyrid-2-yl}propionamide

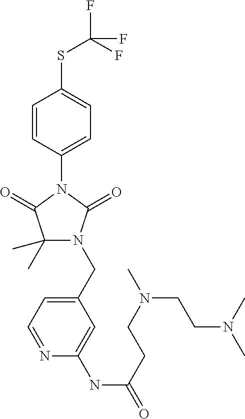 [SMILES: CN(C)CCN(C)CCC(=O)Nc1cc(CN2C(=O)N(c3ccc(SC(F)(F)F)cc3)C(=O)C2(C)C)ccn1]

MS (LC-MS): 566.23, Retention time: 1.37 min.

NMR: 1.45: s, 6H; 2.75: s, 6H; 2.8 to 3.4: broad m, 8H; 7.20: m, 1H; 7.65: d, 2H; 7.85: d, 2H; 8.15: s, 1H; 8.30: m, 1H.

The synthesis is described in Scheme 1.

### Example 1-103: 5,5-dimethyl-1-[2-(5-methylpyrid-2-ylamino)pyrid-4-ylmethyl]-3-(4-trifluoromethylsulfanylphenyl)imidazolidine-2,4-dione

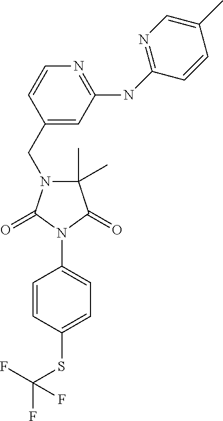 [SMILES: Cc1ccc(Nc2cc(CN3C(=O)N(c4ccc(SC(F)(F)F)cc4)C(=O)C3(C)C)ccn2)nc1]

MS (LC-MS): 501.14, Retention time: 1.83 min.

NMR: 1.45: s, 6H; 2.3: s, 3H; 4.7: s, 2H; 7.3: m, 3H; 7.7: d, 2H; 7.9: d, 2H; 8.2: m, 1H; 8.35: m, 1H.

The synthesis is described in Scheme 1.

### Example 1-104: N-{4-[5,5-dimethyl-2,4-dioxo-3-(4-trifluoro-methylsulfanylphenyl)imidazolidin-1-ylmethyl]pyrid-2-yl}-3,5-dimethoxybenzamide; compound with trifluoroacetic acid

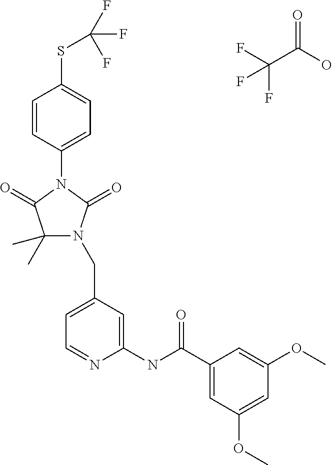 [SMILES: COc1cc(OC)cc(C(=O)Nc2cc(CN3C(=O)N(c4ccc(SC(F)(F)F)cc4)C(=O)C3(C)C)ccn2)c1 ; O=C(O)C(F)(F)F]

MS (LC-MS): 574.78, Retention time: 2.34 min.

NMR: 1.45: s, 6H; 2.3: s, 3H; 4.7: s, 2H; 6.7: s, 2H; 7.2: m, 2H; 7.25: m, 1H; 7.7: d, 2H; 7.9: d, 2H; 8.2: m, 1H; 8.3: m, 1H.

The synthesis is described in Scheme 1.

### Example 1-105: 2-(benzylmethylamino)-N-{4-[5,5-dimethyl-2,4-dioxo-3-(4-trifluoromethylsulfanylphenyl)imidazolidin-1-ylmethyl]pyrid-2-yl}acetamide; compound with trifluoroacetic acid

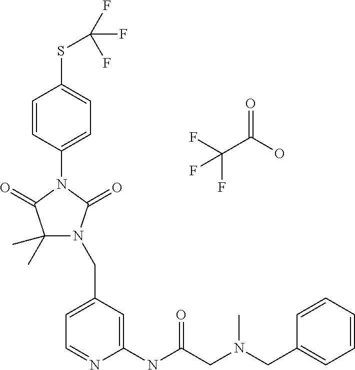 [SMILES: CN(CC(=O)Nc1cc(CN2C(=O)N(c3ccc(SC(F)(F)F)cc3)C(=O)C2(C)C)ccn1)Cc1ccccc1 ; O=C(O)C(F)(F)F]

MS (LC-MS): 574.78, Retention time: 2.34 min.

NMR: 1.45: s, 6H; 2.8: s, 4.0 to 4.4: m, 4H; 4.7: s, 2H; 7.2: m, 1H; 7.4: m, 2H; 7.5: m, 2H; 7.7: d, 2H; 7.9: d, 2H; 8.1: m, 1H; 8.3: m, 1H.

The synthesis is described in Scheme 1.

### Example 1-106: 5,5-dimethyl-1-[2-(pyrazin-2-ylamino)pyrid-4-ylmethyl]-3-(4-trifluoromethylsulfanylphenyl)imidazolidine-2,4-dione; compound with trifluoroacetic acid

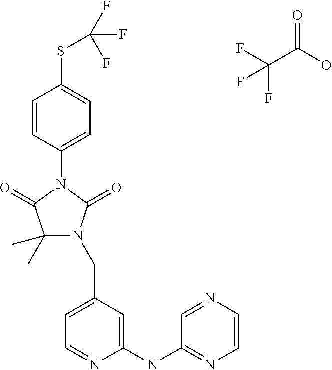 [SMILES: CC1(C)C(=O)N(c2ccc(SC(F)(F)F)cc2)C(=O)N1Cc1ccnc(Nc2cnccn2)c1 ; O=C(O)C(F)(F)F]

MS (LC-MS): 488.12, Retention time: 1.63.

NMR: 1.45: s, 6H; 4.7: s, 2H; 7.1: m, 1H; 7.6: m, 1H; 7.65: d, 2H; 7.9: d, 2H; 8.2: s, 1H; 8.3: m, 2H; 9.0: s, 1H.

The synthesis is described in Scheme 1.

### Example 1-107: N-{4-[5,5-dimethyl-2,4-dioxo-3-(4-trifluoro-methylsulfanylphenyl)imidazolidin-1-ylmethyl]pyrid-2-yl}-2-phenylbutyramide; compound with trifluoroacetic acid

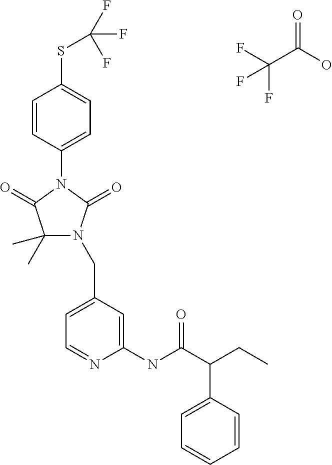 [SMILES: CCC(C(=O)Nc1cc(CN2C(=O)N(c3ccc(SC(F)(F)F)cc3)C(=O)C2(C)C)ccn1)c1ccccc1 ; O=C(O)C(F)(F)F]

MS (LC-MS): 566.18, Retention time: 2.43.

NMR: 0.7: t, 3H; 1.4: s, 6H; 1.7: m, 1H; 2.05: m, 1H; 4.6: s, 2H; 7.1: s, 1H; 7.25: m, 1H; 7.3: m, 1H; 7.4: m, 2H; 7.65: d, 2H; 7.85: d, 2H; 8.15: s, 1H; 8.25: m, 1H.

The synthesis is described in Scheme 1.

### Example 1-108: N-{4-[5,5-dimethyl-2,4-dioxo-3-(4-trifluoro-methylsulfanylphenyl)imidazolidin-1-ylmethyl]pyrid-2-yl}-3-(3-methylpiperid-1-yl)propionamide; compound with trifluoroacetic acid

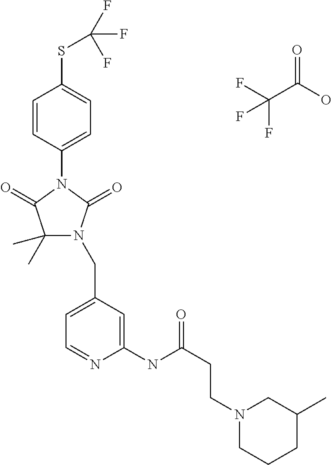 [SMILES: CC1CCCN(CCC(=O)Nc2cc(CN3C(=O)N(c4ccc(SC(F)(F)F)cc4)C(=O)C3(C)C)ccn2)C1 ; O=C(O)C(F)(F)F]

MS (LC-MS): 563.22, Retention time: 1.62.

The synthesis is described in Scheme 1.

### Example 1-109: 1-[2-(4-methoxyphenylamino)pyrid-4-ylmethyl]-5,5-dimethyl-3-(4-trifluoromethylsulfanylphenyl)imidazolidine-2,4-dione

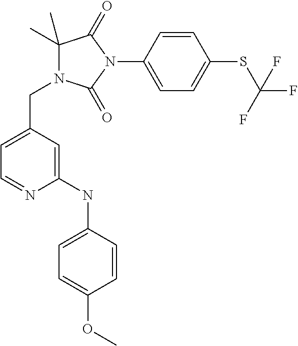 [SMILES: COc1ccc(Nc2cc(CN3C(=O)N(c4ccc(SC(F)(F)F)cc4)C(=O)C3(C)C)ccn2)cc1]

MS (LC-MS): 517.1; Retention time: 1.74.

NMR: 1.4: s, 6H; 4.6: s, 2H; 6.85: broad s, 1H; 7.0: m, 1H; 7.4: m, 2H; 7.6: d, 2H; 7.9: d, 2H; 8.95: s, 1H.

The synthesis is described in Scheme 1.

### Example 1-110: 5,5-dimethyl-1-[2-(2-oxopyrrolidin-1-yl)pyrid-4-ylmethyl]-3-(4-trifluoromethylsulfanylphenyl)imidazolidine-2,4-dione

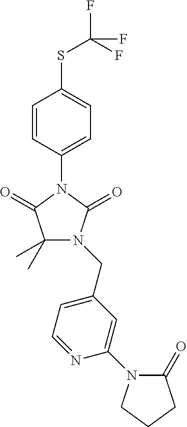 [SMILES: CC1(C)C(=O)N(c2ccc(SC(F)(F)F)cc2)C(=O)N1Cc1ccnc(N2CCCC2=O)c1]

MS (LC-MS): 478.13; Retention time: 1.83.

The synthesis is described in Scheme 1.

### Example 1-111: {4-[5,5-dimethyl-2,4-dioxo-3-(4-trifluoro-methylsulfanylphenyl)imidazolidin-1-ylmethyl]pyrid-2-yl}-pyrazine-2-carboxamide; compound with trifluoroacetic acid

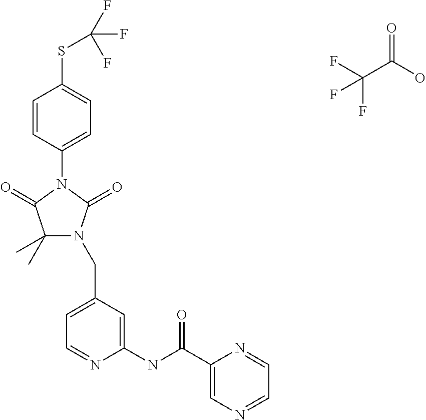 [SMILES: CC1(C)C(=O)N(c2ccc(SC(F)(F)F)cc2)C(=O)N1Cc1ccnc(NC(=O)c2cnccn2)c1 ; O=C(O)C(F)(F)F]

MS (LC-MS): 516.12; Retention time: 2.42.

NMR: 1.4: s, 6H; 4.7: s, 2H; 7.3: m, 1H; 7.65: d, 2H; 7.95: d, 2H; 8.3: s, 1H; 8.4: s, 1H; 8.7: s, 1H; 9.0: s, 1H; 9.4: s, 1H.

The synthesis is described in Scheme 1.

### Example 1-112: N-{4-[5,5-dimethyl-2,4-dioxo-3-(4-trifluoro-methylsulfanylphenyl)imidazolidin-1-ylmethyl]pyrid-2-yl}-2,2-dimethylpropionamide; compound with trifluoroacetic acid

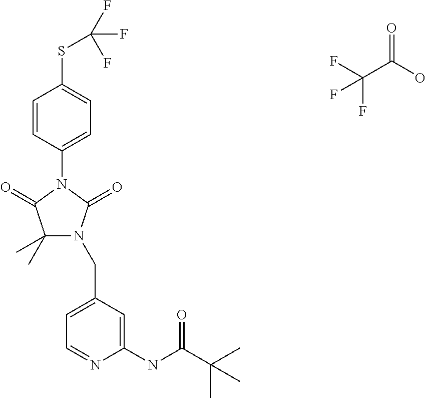 [SMILES: CC(C)(C)C(=O)Nc1cc(CN2C(=O)N(c3ccc(SC(F)(F)F)cc3)C(=O)C2(C)C)ccn1 ; O=C(O)C(F)(F)F]

MS (LC-MS): 494.16; Retention time: 2.48.

NMR: 1.2: s, 9H; 1.4: s, 6H; 4.7: s, 2H; 7.2: m, 1H; 7.65: d, 2H; 7.90: d, 2H; 8.1: s, 1H; 8.3: s, 1H.

The synthesis is described in Scheme 1.

### Example 1-113: {4-[5,5-dimethyl-2,4-dioxo-3-(4-trifluoro-methylsulfanylphenyl)imidazolidin-1-ylmethyl]pyrid-2-yl}-thiophene-2-carboxamide; compound with trifluoroacetic acid

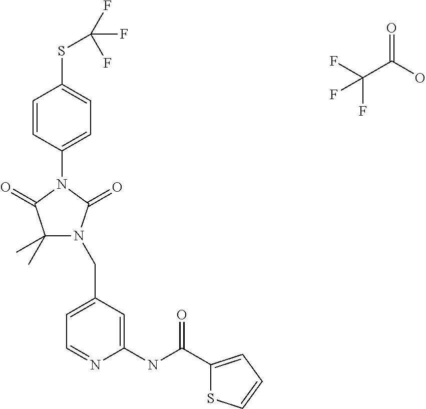 [SMILES: CC1(C)C(=O)N(c2ccc(SC(F)(F)F)cc2)C(=O)N1Cc1ccnc(NC(=O)c2cccs2)c1 ; O=C(O)C(F)(F)F]

MS (LC-MS): 520.09; Retention time: 2.14.

NMR: 1.4: s, 6H; 4.7: s, 2H; 7.2: m, 2H; 7.65: d, 2H; 7.8: m, 3H; 8.2: s, 1H; 8.25: s, 1H; 8.4: m, 1H.

The synthesis is described in Scheme 1.

### Example 1-114: N-{4-[5,5-dimethyl-2,4-dioxo-3-(4-trifluoro-methylsulfanylphenyl)imidazolidin-1-ylmethyl]pyrid-2-yl}-4-methylbenzamide; compound with trifluoroacetic acid

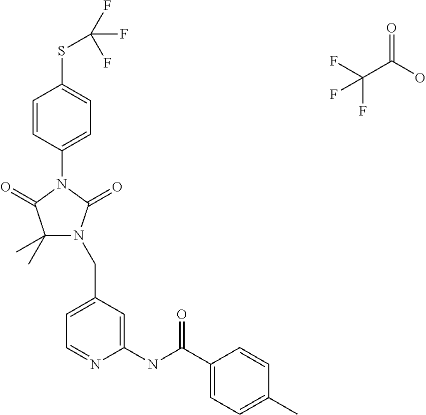 [SMILES: Cc1ccc(C(=O)Nc2cc(CN3C(=O)N(c4ccc(SC(F)(F)F)cc4)C(=O)C3(C)C)ccn2)cc1 ; O=C(O)C(F)(F)F]

MS (LC-MS): 528.16; Retention time: 2.23.

NMR: 1.4: s, 6H; 4.7: s, 2H; 7.2: m, 1H; 7.3: d, 2H; 7.7: d, 2H; 7.85: d, 2H; 7.95: d, 2H; 8.2: s, 1H; 8.3: s, 1H.

The synthesis is described in Scheme 1.

### Example 1-115: N-{4-[5,5-dimethyl-2,4-dioxo-3-(4-trifluoro-methylsulfanylphenyl)imidazolidin-1-ylmethyl]pyrid-2-yl}-3-(3,5-dimethylpiperid-1-yl)propionamide; compound with trifluoroacetic acid

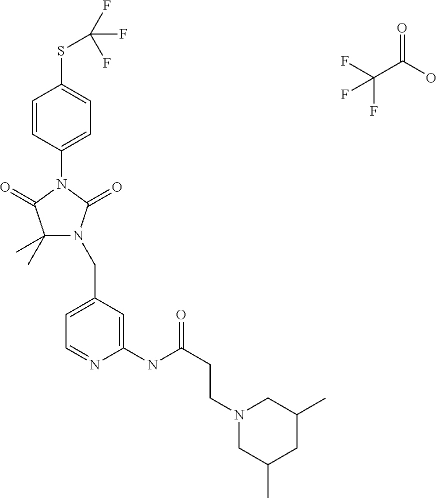 [SMILES: CC1CC(C)CN(CCC(=O)Nc2cc(CN3C(=O)N(c4ccc(SC(F)(F)F)cc4)C(=O)C3(C)C)ccn2)C1 ; O=C(O)C(F)(F)F]

MS (LC-MS): 577.23; Retention time: 1.72.

The synthesis is described in Scheme 1.

### Example 1-116: N-{4-[5,5-dimethyl-2,4-dioxo-3-(4-trifluoro-methylsulfanylphenyl)imidazolidin-1-ylmethyl]pyrid-2-yl}-3-(4-pyrid-2-ylpiperazin-1-yl)propionamide; compound with trifluoroacetic acid

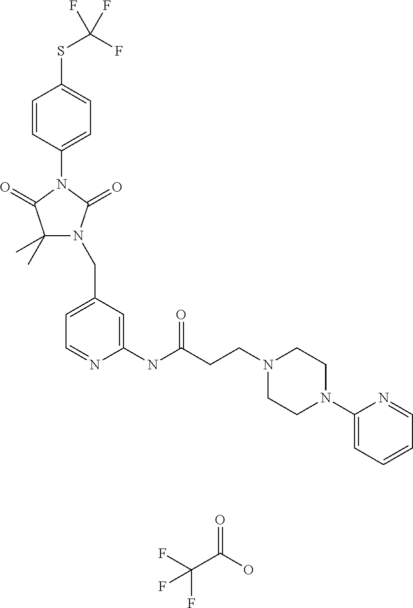 [SMILES: CC1(C)C(=O)N(c2ccc(SC(F)(F)F)cc2)C(=O)N1Cc1ccnc(NC(=O)CCN2CCN(c3ccccn3)CC2)c1 ; O=C(O)C(F)(F)F]

MS (LC-MS): 627.22; Retention time: 1.50.

NMR: 1.4: s, 6H; 2.9: m, 2H; 3.1: m, 2H; 3.4: m, 2H; 3.6: m, 2H; 4.4: m, 2H; 4.65: s, 2H; 6.7: m, 1H; 6.95: d, 1H; 7.2: m, 1H; 7.7: d, 2H; 7.9: d, 2H; 8.15: m, 2H; 8.3: m, 1H.

The synthesis is described in Scheme 1.

### Example 1-117: 5,5-dimethyl-1-[2-(5-trifluoromethylpyrid-2-ylamino)pyri ylmethyl]-3-(4-trifluoromethylsulfanylphenyl)imidazolidine-2,4-dione

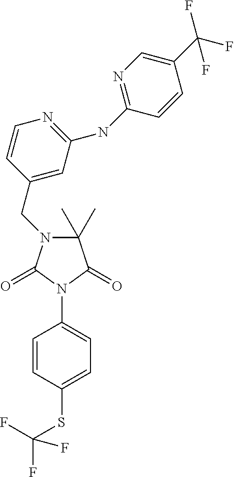 [SMILES: CC1(C)C(=O)N(c2ccc(SC(F)(F)F)cc2)C(=O)N1Cc1ccnc(Nc2ccc(C(F)(F)F)cn2)c1]

MS (LC-MS): 555.51; Retention time: 1.78.

The synthesis is described in Scheme 1.

### Example 1-118: N-{4-[5,5-dimethyl-2,4-dioxo-3-(4-trifluoro-methylsulfanylphenyl)imidazolidin-1-ylmethyl]pyrid-2-yl}-3-methoxybenzamide; compound with trifluoroacetic acid

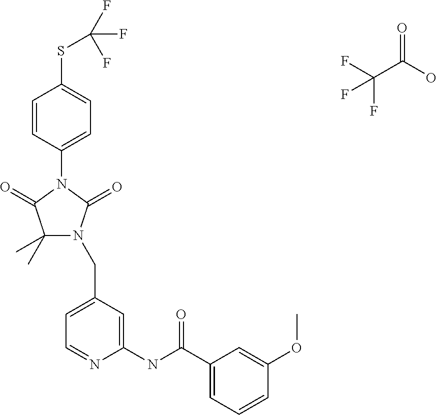 [SMILES: COc1cccc(C(=O)Nc2cc(CN3C(=O)N(c4ccc(SC(F)(F)F)cc4)C(=O)C3(C)C)ccn2)c1 ; O=C(O)C(F)(F)F]

MS (LC-MS): 544.14; Retention time: 2.15.

The synthesis is described in Scheme 1.

### Example 1-119: 1-(3,5-dichlorophenyl)-3-{4-[5,5-dimethyl-2,4-dioxo-3-(4-trifluoromethylsulfanylphenyl)imidazolidin-1-ylmethyl]pyrid-2-yl}urea

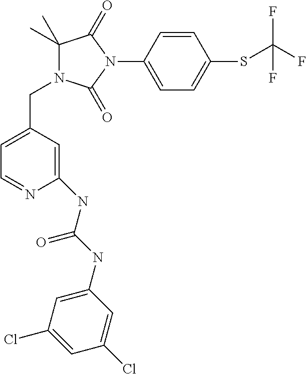 [SMILES: CC1(C)C(=O)N(c2ccc(SC(F)(F)F)cc2)C(=O)N1Cc1ccnc(NC(=O)Nc2cc(Cl)cc(Cl)c2)c1]

MS (LC-MS): 597.06; Retention time: 2.15 min.

The synthesis is described in Scheme 2.

### Example 1-120: Methyl 3-(3-{4-[5,5-dimethyl-2,4-dioxo-3-(4-trifluoromethylsulfanylphenyl)imidazolidin-1-ylmethyl]pyrid-2-yl}thioureido)benzoate

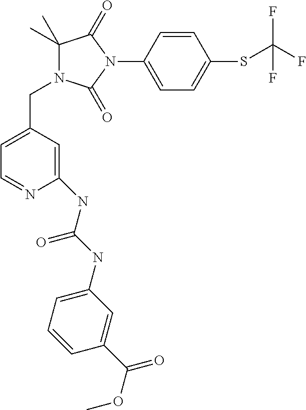 [SMILES: COC(=O)c1cccc(NC(=O)Nc2cc(CN3C(=O)N(c4ccc(SC(F)(F)F)cc4)C(=O)C3(C)C)ccn2)c1]

MS (LC-MS): 603.12; Retention time: 2.81 min.

The synthesis is described in Scheme 2.

### Example 1-121: 1-{4-[5,5-dimethyl-2,4-dioxo-3-(4-trifluoro-methylsulfanylphenyl)imidazolidin-1-ylmethyl]pyrid-2-yl}-3-phenylurea

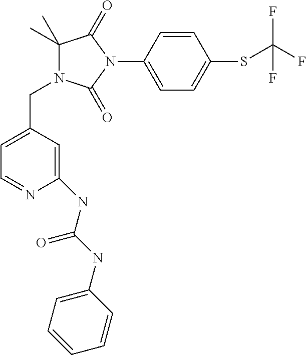 [SMILES: CC1(C)C(=O)N(c2ccc(SC(F)(F)F)cc2)C(=O)N1Cc1ccnc(NC(=O)Nc2ccccc2)c1]

MS (LC-MS): 529.14; Retention time: 2.33 min.

NMR: 1.50: s, 6H; 4.65: s, 1H; 7.0: m, 2H; 7.30: m, 2H; 7.5: m, 3H; 7.65: d, 2H; 7.85: d, 2H; 8.25: d, 1H; 9.4: s, 1H.

The synthesis is described in Scheme 2.

### Example 1-122: 1-(2,4-dichlorophenyl)-3-{4-[5,5-dimethyl-2,4-dioxo-3-(4-trifluoromethylsulfanylphenyl)imidazolidin-1-ylmethyl]pyrid-2-yl}urea

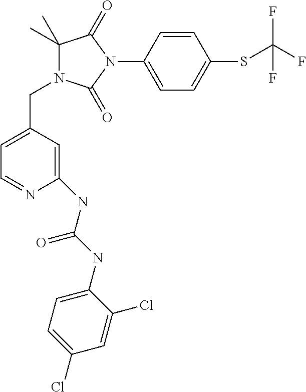 [SMILES: CC1(C)C(=O)N(c2ccc(SC(F)(F)F)cc2)C(=O)N1Cc1ccnc(NC(=O)Nc2ccc(Cl)cc2Cl)c1]

MS (LC-MS): 597.06; Retention time: 3.00 min.

The synthesis is described in Scheme 2.

### Example 1-123: 1-(3-chlorophenyl)-3-{4-[5,5-dimethyl-2,4-dioxo-3-(4-trifluoromethylsulfanylphenyl)imidazolidin-1-ylmethyl]pyrid-2-yl}thiourea

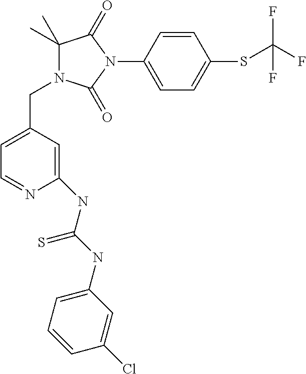 [SMILES: CC1(C)C(=O)N(c2ccc(SC(F)(F)F)cc2)C(=O)N1Cc1ccnc(NC(=S)Nc2cccc(Cl)c2)c1]

MS (LC-MS): 579.08; Retention time: 3.05 min.

The synthesis is described in Scheme 2.

### Example 1-124: Methyl 2-(3-{4-[5,5-dimethyl-2,4-dioxo-3-(4-trifluoromethylsulfanylphenyl)imidazolidin-1-ylmethyl]pyrid-2-yl}thioureido)benzoate

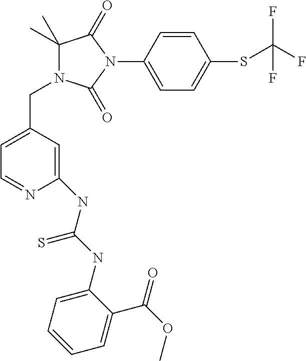 [SMILES: COC(=O)c1ccccc1NC(=S)Nc1cc(CN2C(=O)N(c3ccc(SC(F)(F)F)cc3)C(=O)C2(C)C)ccn1]

MS (LC-MS): 603.12; Retention time: 2.85 min.

The synthesis is described in Scheme 2.

### Example 1-125: 3,5-diacetoxy-2-acetoxymethyl-6-(3-{4-[5,5-dimethyl-2,4-dioxo-3-(4-trifluoromethylsulfanylphenyl)imidazolidin-1-ylmethyl]pyrid-2-yl}thioureido)tetrahydropyran-4-yl acetate

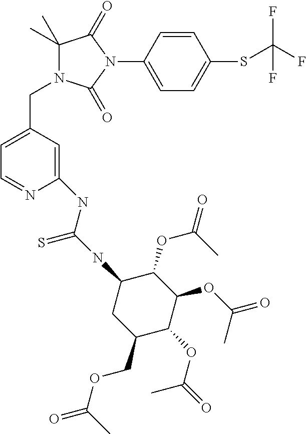 [SMILES: CC(=O)OC[C@H]1C[C@@H](NC(=S)Nc2cc(CN3C(=O)N(c4ccc(SC(F)(F)F)cc4)C(=O)C3(C)C)ccn2)[C@H](OC(C)=O)[C@@H](OC(C)=O)[C@@H]1OC(C)=O]

MS (LC-MS): 799.18, Retention time: 2.60 min.

The synthesis is described in Scheme 2.

### Example 1-126: 1-(4-dimethylaminophenyl)-3-{4-[5,5-dimethyl-2,4-dioxo-3-(4-trifluoromethylsulfanylphenyl)imidazolidin-1-ylmethyl]pyrid-2-yl}thiourea

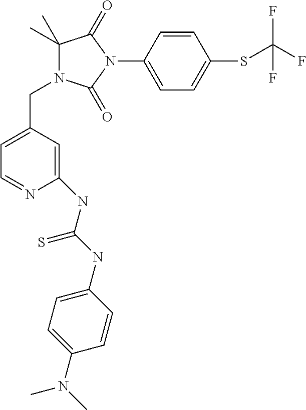 [SMILES: CN(C)c1ccc(NC(=S)Nc2cc(CN3C(=O)N(c4ccc(SC(F)(F)F)cc4)C(=O)C3(C)C)ccn2)cc1]

MS (LC-MS): 588.16; Retention time: 1.99.

NMR: 1.4: s, 6H; 2.9: s, 6H; 4.7: s, 2H; 6.7: d, 2H; 7.1: m, 1H; 7.25: s, 1H; 7.4: d, 2H; 7.7: d, 2H; 7.9: d, 2H; 8.25: m, 1H.

The synthesis is described in Scheme 2.

### Example 1-127: 1-(2,4-dimethoxyphenyl)-3-{4-[5,5-dimethyl-2,4-dioxo-3-(4-trifluoromethylsulfanylphenyl)imidazolidin-1-ylmethyl]pyrid-2-yl}urea

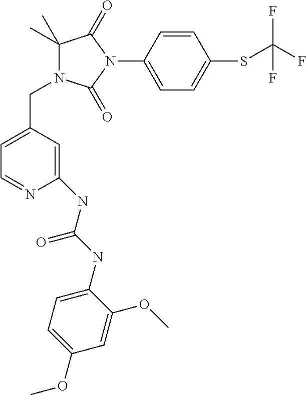 [SMILES: COc1ccc(NC(=O)Nc2cc(CN3C(=O)N(c4ccc(SC(F)(F)F)cc4)C(=O)C3(C)C)ccn2)c(OC)c1]

MS (LC-MS): 589.60; Retention time: 1.98.

The synthesis is described in Scheme 2.

### Example 1-128: 3-(3-{4-[5,5-dimethyl-2,4-dioxo-3-(4-trifluoromethylsulfanylphenyl)imidazolidin-1-ylmethyl]pyrid-2-yl}thioureido)benzoic acid

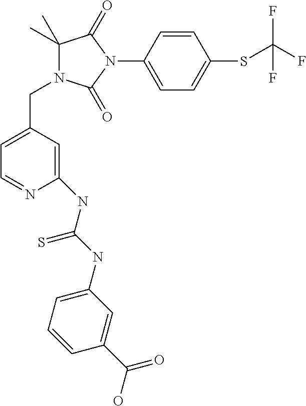 [SMILES: CC1(C)C(=O)N(c2ccc(SC(F)(F)F)cc2)C(=O)N1Cc1ccnc(NC(=S)Nc2cccc(C(=O)O)c2)c1]

MS (LC-MS): 589.11; Retention time: 2.30.

The synthesis is described in Scheme 2.

### Example 1-129: 1-{4-[5,5-dimethyl-2,4-dioxo-3-(4-trifluorometh-ylsulfanylphenyl)imidazolidin-1-ylmethyl]pyrid-2-yl}-3-(2-methoxyphenyl)urea

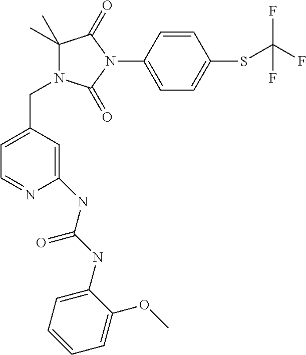 [SMILES: COc1ccccc1NC(=O)Nc1cc(CN2C(=O)N(c3ccc(SC(F)(F)F)cc3)C(=O)C2(C)C)ccn1]

MS (LC-MS): 559.15; Retention time: 1.88.

The synthesis is described in Scheme 2.

### Example 1-130: 1-(2-aminopyrid-4-ylmethyl)-5,5-dimethyl-3-(4-trifluoromethylsulfanylphenyl)imidazolidine-2,4-dione

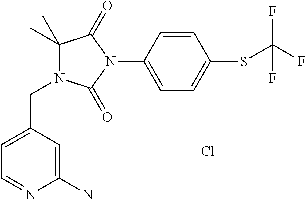 [SMILES: CC1(C)C(=O)N(c2ccc(SC(F)(F)F)cc2)C(=O)N1Cc1ccnc(N)c1 ; Cl]

MS (LC-MS): 410.10; Retention time: 1.57.

NMR: 1.4: s, 6H; 4.6: s, 2H; 6.9: d, 1H; 7.0: s, 1H; 7.7: d, 2H; 7.85: d, 2H; 7.95: d, 1H; 8.0: broad s, 1H.

The synthesis is described in Scheme 2.

### Example 1-131: 1-(2,6-dichloropyrid-4-yl)-3-{4-[5,5-dimethyl-2,4-dioxo-3-(4-trifluoromethylsulfanylphenyl)imidazolidin-1-ylmethyl]pyrid-2-yl}urea

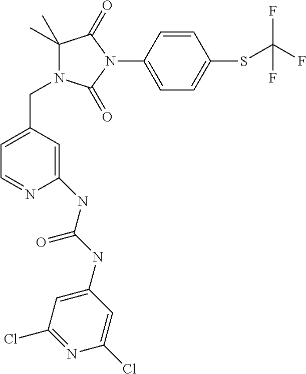 [SMILES: CC1(C)C(=O)N(c2ccc(SC(F)(F)F)cc2)C(=O)N1Cc1ccnc(NC(=O)Nc2cc(Cl)nc(Cl)c2)c1]

MS (LC-MS): 599.42; Retention time: 2.73 min.

NMR: 1.40, s, 6H; 4.65 s, 2H; 7.15: m, 1H; 7.65: d, 2H; 7.85: d, 2H; 8.15: s, 1H; 8.25: m, 1H.

The synthesis is described in Scheme 2.

### Example 1-132: 1-(2,6-dichlorophenyl)-3-{4-[5,5-dimethyl-2,4-dioxo-3-(4-trifluoromethylsulfanylphenyl)imidazolidin-1-ylmethyl]pyrid-2-yl}urea

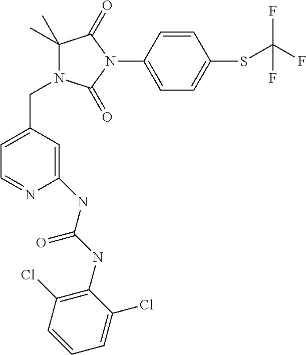 [SMILES: CC1(C)C(=O)N(c2ccc(SC(F)(F)F)cc2)C(=O)N1Cc1ccnc(NC(=O)Nc2c(Cl)cccc2Cl)c1]

MS (LC-MS): 597.06; Retention time: 2.44 min.

The synthesis is described in Scheme 2.

### Example 1-133: 1-(2,3-dichlorophenyl)-3-{4-[5,5-dimethyl-2,4-dioxo-3-(4-trifluoromethylsulfanylphenyl)imidazolidin-1-ylmethyl]pyrid-2-yl}urea

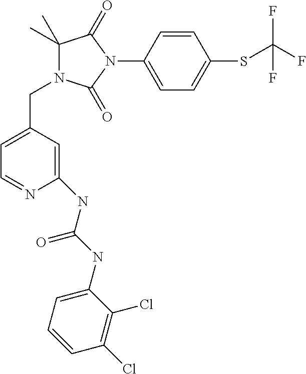 [SMILES: CC1(C)C(=O)N(c2ccc(SC(F)(F)F)cc2)C(=O)N1Cc1ccnc(NC(=O)Nc2cccc(Cl)c2Cl)c1]

MS (LC-MS): 597.06; Retention time: 2.08 min.

The synthesis is described in Scheme 2.

### Example 1-134: 1-{4-[5,5-dimethyl-2,4-dioxo-3-(4-trifluoro-methylsulfanylphenyl)imidazolidin-1-ylmethyl]pyrid-2-yl}-3-pyrid-3-ylthiourea

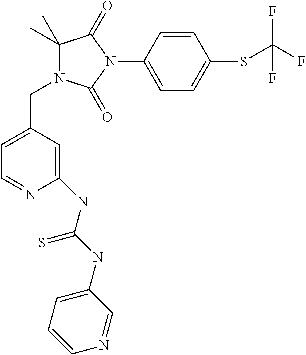 [SMILES: CC1(C)C(=O)N(c2ccc(SC(F)(F)F)cc2)C(=O)N1Cc1ccnc(NC(=S)Nc2cccnc2)c1]

MS (LC-MS): 546.11; Retention time: 1.89 min.

The synthesis is described in Scheme 2.

### Example 1-135: 1-[2-chloro-6-(4-methylthiazol-2-ylamino)pyrid-4-ylmethyl]-5,5-dimethyl-3-(4-trifluoromethylsulfanylphenyl)imidazolidine-2,4-dione

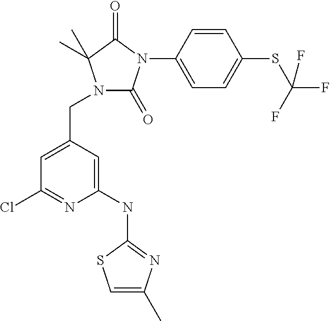 [SMILES: Cc1csc(Nc2cc(CN3C(=O)N(c4ccc(SC(F)(F)F)cc4)C(=O)C3(C)C)cc(Cl)n2)n1]

MS (LC-MS): 541.06; Retention time: 2.49 min.

The synthesis is described in Scheme 3.

### Example 1-136: N-{6-chloro-4-[5,5-dimethyl-2,4-dioxo-3-(4-trifluoromethylsulfanylphenyl)imidazolidin-1-ylmethyl]pyrid-2-yl}-2-phenylbutyramide

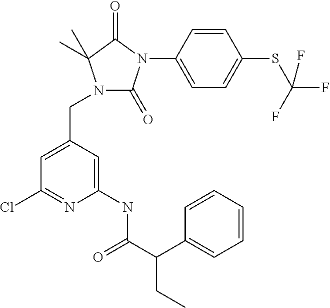 [SMILES: CCC(C(=O)Nc1cc(CN2C(=O)N(c3ccc(SC(F)(F)F)cc3)C(=O)C2(C)C)cc(Cl)n1)c1ccccc1]

MS (LC-MS): 541.06; Retention time: 2.49 min.

The synthesis is described in Scheme 3.

### Example 1-137: N-{6-acetylamino-4-[5,5-dimethyl-2,4-dioxo-3-(4-trifluoromethylsulfanylphenyl)imidazolidin-1-ylmethyl]pyrid-2-yl}acetamide

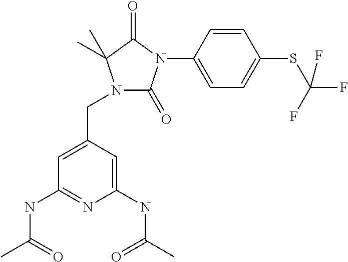 [SMILES: CC(=O)Nc1cc(CN2C(=O)N(c3ccc(SC(F)(F)F)cc3)C(=O)C2(C)C)cc(NC(C)=O)n1]

MS (LC-MS): 509.13; Retention time: 1.73 min.

The synthesis is described in Scheme 3.

### Example 1-138: 1-[2-chloro-6-(4-methylpyrid-2-ylamino)pyrid-4-ylmethyl]-5,5-dimethyl-3-(4-trifluoromethylsulfanylphenyl)imidazolidine-2,4-dione

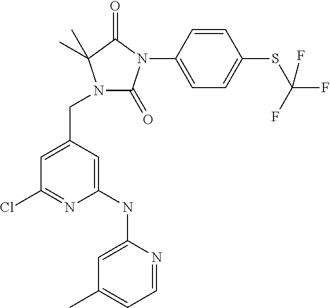 [SMILES: Cc1ccnc(Nc2cc(CN3C(=O)N(c4ccc(SC(F)(F)F)cc4)C(=O)C3(C)C)cc(Cl)n2)c1]

MS (LC-MS): 535.11; Retention time: 1.90 min.

The synthesis is described in Scheme 3.

### Example 1-139: 1-[2,6-bis(3-methoxyphenylamino)pyrid-4-ylmethyl]-5,5-dimethyl-3-(4-trifluoromethoxyphenyl)imidazolidine-2,4-dione; compound with trifluoroacetic acid

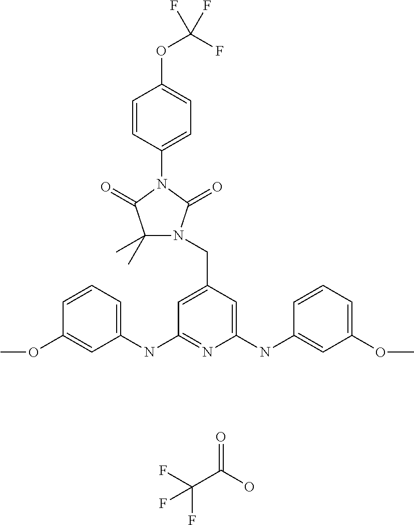

MS (LC-MS): 621.22; Retention time: 1.95 min.

The synthesis is described in Scheme 3.

### Example 1-140: 1-[2,6-bis(2,4-dimethoxyphenylamino)pyrid-4-ylmethyl]-5,5-dimethyl-3-(4-trifluoromethoxyphenyl)imidazolidine-2,4-dione; compound with trifluoroacetic acid

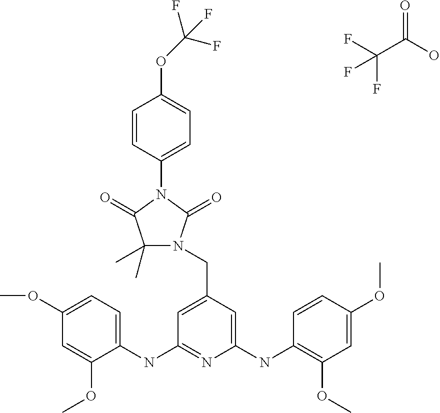

MS (LC-MS): 681.24; Retention time: 1.83 min.

The synthesis is described in Scheme 3.

### Example 1-141: 1-[2,6-bis(4-methoxyphenylamino)pyrid-4-ylmethyl]-5,5-dimethyl-3-(4-trifluoromethoxyphenyl)imidazolidine-2,4-dione; compound with trifluoroacetic acid

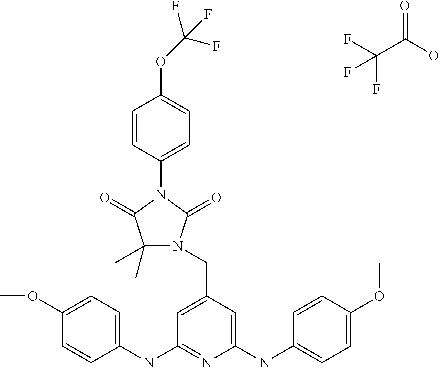

MS (LC-MS): 621.22; Retention time: 1.63 min.

The synthesis is described in Scheme 3.

### Example 1-142: N-{4-[5,5-dimethyl-2,4-dioxo-3-(4-trifluoro-methoxyphenyl)imidazolidin-1-ylmethyl]-6-estobutyrylaminopyrid-2-yl}isobutyramide; compound with trifluoroacetic acid

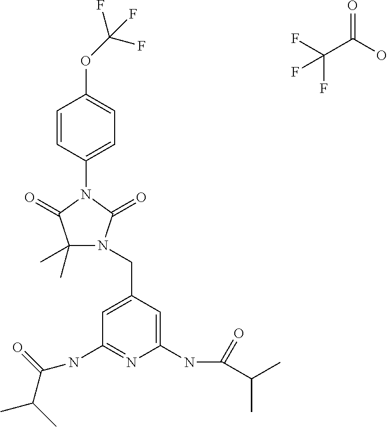 [SMILES: CC(C)C(=O)Nc1cc(CN2C(=O)N(c3ccc(OC(F)(F)F)cc3)C(=O)C2(C)C)cc(NC(=O)C(C)C)n1 ; O=C(O)C(F)(F)F]

MS (LC-MS): 549.22; Retention time: 1.83 min.

The synthesis is described in Scheme 3.

### Example 1-143: 1-[2-chloro-6-(pyrid-4-ylamino)pyrid-4-ylmethyl]-5,5-dimethyl-3-(4-trifluoromethoxyphenyl)imidazolidine-2,4-dione; compound with trifluoroacetic acid

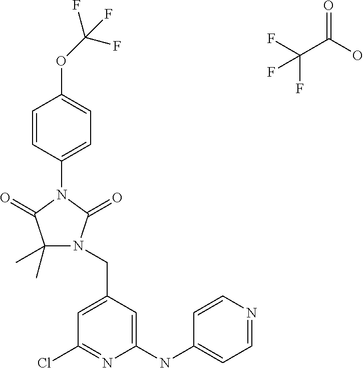 [SMILES: CC1(C)C(=O)N(c2ccc(OC(F)(F)F)cc2)C(=O)N1Cc1cc(Cl)nc(Nc2ccncc2)c1 ; O=C(O)C(F)(F)F]

MS (LC-MS): 563.19; Retention time: 2.61 min.

The synthesis is described in Scheme 3.

### Example 1-144: 1-[2-chloro-6-(pyrid-4-ylamino)pyrid-4-ylmethyl]-5,5-dimethyl-3-(4-trifluoromethylsulfanylphenyl)imidazolidine-2,4-dione

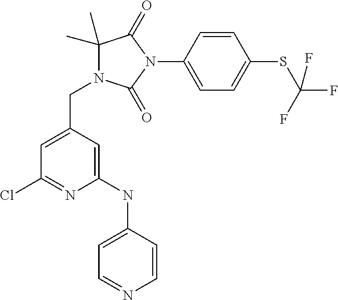 [SMILES: CC1(C)C(=O)N(c2ccc(SC(F)(F)F)cc2)C(=O)N1Cc1cc(Cl)nc(Nc2ccncc2)c1]

MS (LC-MS): 521.09; Retention time: 1.91 min.

The synthesis is described in Scheme 3.

### Example 1-145: N-{6-chloro-4-[5,5-dimethyl-2,4-dioxo-3-(4-trifluoromethylsulfanylphenyl)imidazolidin-1-ylmethyl]pyridin-2-yl}propionamide

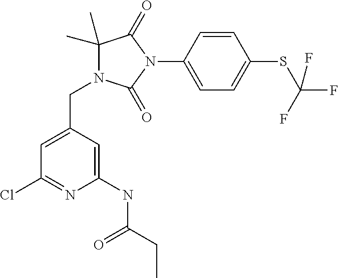 [SMILES: CCC(=O)Nc1cc(CN2C(=O)N(c3ccc(SC(F)(F)F)cc3)C(=O)C2(C)C)cc(Cl)n1]

MS (LC-MS): 500.09; Retention time: 2.46.

The synthesis is described in Scheme 3.

### Example 1-146: N-{6-chloro-4-[5,5-dimethyl-2,4-dioxo-3-(4-trifluoromethylsulfanylphenyl)imidazolidin-1-ylmethyl]pyrid-2-yl}acetamide

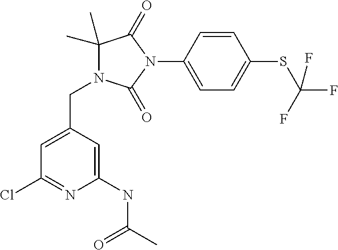 [SMILES: CC(=O)Nc1cc(CN2C(=O)N(c3ccc(SC(F)(F)F)cc3)C(=O)C2(C)C)cc(Cl)n1]

MS (LC-MS): 486.90; Retention time: 2.15.

The synthesis is described in Scheme 3.

### Example 1-147: 1-[2-chloro-6-(pyrid-3-ylamino)pyrid-4-ylmethyl]-5,5-dimethyl-3-(4-trifluoromethoxyphenyl)imidazolidine-2,4-dione; compound with trifluoroacetic acid

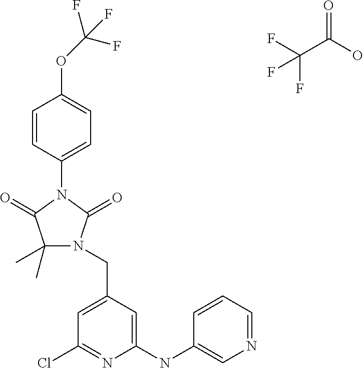 [SMILES: CC1(C)C(=O)N(c2ccc(OC(F)(F)F)cc2)C(=O)N1Cc1cc(Cl)nc(Nc2cccnc2)c1 ; O=C(O)C(F)(F)F]

MS (LC-MS): 505.11; Retention time: 2.27.

The synthesis is described in Scheme 3.

### Example 1-148: {6-chloro-4-[5,5-dimethyl-2,4-dioxo-3-(4-trifluoromethylsulfanylphenyl)imidazolidin-1-ylmethyl]pyrid-2-yl}cyclopropanecarboxamide

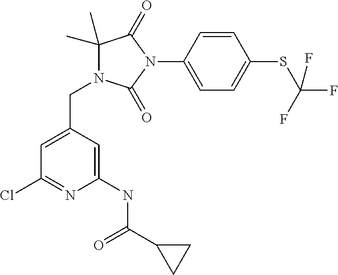 [SMILES: CC1(C)C(=O)N(c2ccc(SC(F)(F)F)cc2)C(=O)N1Cc1cc(Cl)nc(NC(=O)C2CC2)c1]

MS (LC-MS): 512.09; Retention time: 2.32.

The synthesis is described in Scheme 3.

### Example 1-149: N-{6-chloro-4-[5,5-dimethyl-2,4-dioxo-3-(4-trifluoromethylsulfanylphenyl)imidazolidin-1-ylmethyl]pyrid-2-yl}isobutyramide

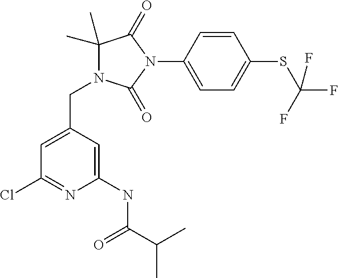 [SMILES: CC(C)C(=O)Nc1cc(CN2C(=O)N(c3ccc(SC(F)(F)F)cc3)C(=O)C2(C)C)cc(Cl)n1]

MS (LC-MS): 514.11; Retention time: 1.63.

The synthesis is described in Scheme 3.

### Example 1-150: 1-[2,6-bis(pyrid-2-ylamino)pyrid-4-ylmethyl]-5,5-dimethyl-3-(4-trifluoromethoxyphenyl)imidazolidine-2,4-dione; compound with trifluoroacetic acid

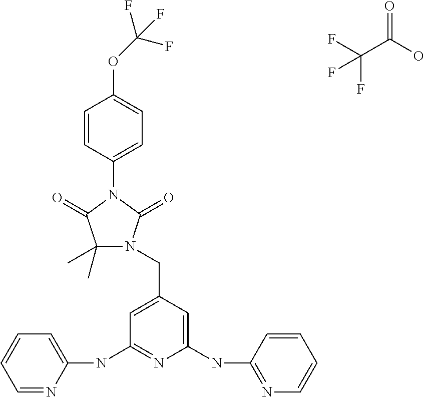 [SMILES: CC1(C)C(=O)N(c2ccc(OC(F)(F)F)cc2)C(=O)N1Cc1cc(Nc2ccccn2)nc(Nc2ccccn2)c1 ; O=C(O)C(F)(F)F]

MS (LC-MS): 563.15; Retention time: 1.70 min.

The synthesis is described in Scheme 3.

### Example 1-151: 1-[2-chloro-6-(pyrid-2-ylamino)pyrid-4-ylmethyl]-5,5-dimethyl-3-(4-trifluoromethylsulfanylphenyl)imidazolidine-2,4-dione

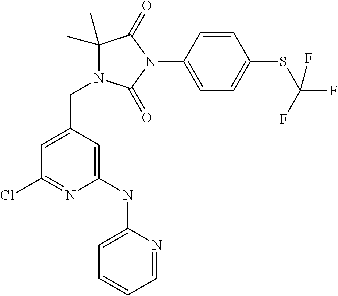 [SMILES: CC1(C)C(=O)N(c2ccc(SC(F)(F)F)cc2)C(=O)N1Cc1cc(Cl)nc(Nc2ccccn2)c1]

MS (LC-MS): 521.09; Retention time: 1.78 min.

The synthesis is described in Scheme 3.

### Example 1-152: 5,5-dimethyl-1-(5-phenyl[1,2,4]oxadiazol-3-ylmethyl)-3-(4-trifluoromethylsulfanylphenyl)imidazolidine-2,4-dione

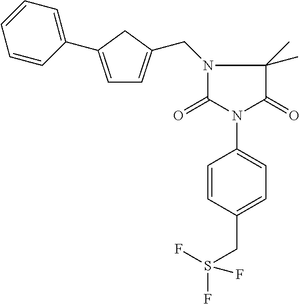 [SMILES: CC1(C)C(=O)N(c2ccc(CS(F)(F)F)cc2)C(=O)N1CC1=CC=C(c2ccccc2)C1]

MS (LC-MS): 462.10; Retention time: 2.77 min.

The synthesis is described in Scheme 4.

### Example 1-153: 1-(2-imidazol-1-ylethyl)-5,5-dimethyl-3-(4-trifluoromethylsulfanylphenyl)imidazolidine-2,4-dione

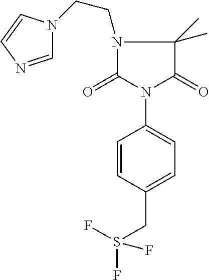 [SMILES: CC1(C)C(=O)N(c2ccc(CS(F)(F)F)cc2)C(=O)N1CCn1ccnc1]

MS (LC-MS): 398.10; Retention time: 1.49 min.

The synthesis is described in Scheme 4.

### Example 1-154: 1-[2-(4-chlorophenyl)oxazol-5-ylmethyl]-5,5-dimethyl-3-(4-trifluoromethylsulfanylphenyl)imidazolidine-2,4-dione

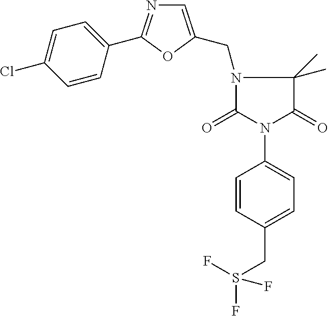 [SMILES: CC1(C)C(=O)N(c2ccc(CS(F)(F)F)cc2)C(=O)N1Cc1cnc(-c2ccc(Cl)cc2)o1]

MS (LC-MS): 495.06; Retention time: 2.99 min.

The synthesis is described in Scheme 4.

### Example 1-155: Preparation of 1-isoquinolin-5-yl-5,5-dimethyl-3-(4-trifluoromethylsulfanylphenyl)imidazolidine-2,4-dione

500 mg (2.4 mmol) of 2-(isoquinolin-5-ylamino)-2-methylpropionitrile, 1.6 mg (0.013 mmol) of benzoic acid and 526 mg (2.4 mmol) of 4-(trifluoromethylthio)phenyl isocyanate in 4 ml of chlorobenzene were refluxed for 2 days. The resulting mixture was filtered and the filtrate was evaporated to dryness. The intermediate imine was separated out by preparative HPLC chromatography (RP 18, acetonitrile, water, 0.01% TFA). 60 mg of the resulting product were stirred for 1 hour at 40° C. with 1N hydrochloric acid. The solvent was evaporated off and the residue was taken up in sodium hydrogen carbonate solution and extracted with ethyl acetate. The combined organic phases were dried and the residue remaining after evaporation was purified by flash chromatography (SiO₂, methylene chloride/methanol=98/2), to give 38 mg of the desired product.

M+H⁺=432.

LC/MS Retention time=1.245.

### Example 1-156: 5,5-dimethyl-1-(5-phenyloxazol-4-ylmethyl)-3-(4-trifluoromethylsulfanylphenyl)imidazolidine-2,4-dione

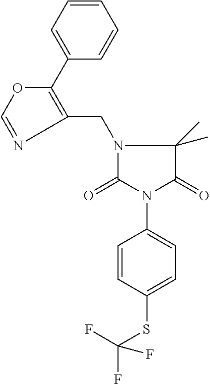 [SMILES: CC1(C)C(=O)N(c2ccc(SC(F)(F)F)cc2)C(=O)N1Cc1ncoc1-c1ccccc1]

MS (LC-MS): 461.10, Retention time: 2.63 min.

The synthesis is described in Scheme 4.

### Example 1-157: 5,5-dimethyl-1-(1-methylpiperid-3-ylmethyl)-3-(4-trifluoromethylsulfanylphenyl)imidazolidine-2,4-dione

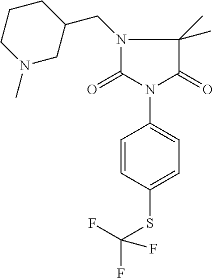 [SMILES: CN1CCCC(CN2C(=O)N(c3ccc(SC(F)(F)F)cc3)C(=O)C2(C)C)C1]

MS (LC-MS): 415.15; Retention time: 1.63.

The synthesis is described in Scheme 4.

### Example 1-158: 5,5-dimethyl-1-(1-methylpiperid-3-ylmethyl)-3-(4-trifluoromethoxyphenyl)imidazolidine-2,4-dione

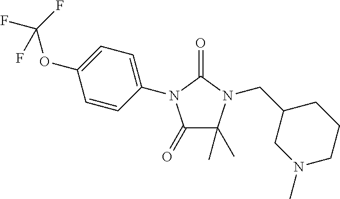 [SMILES: CN1CCCC(CN2C(=O)N(c3ccc(OC(F)(F)F)cc3)C(=O)C2(C)C)C1]

MS (LC-MS): 499.18; Retention time: 1.39.

The synthesis is described in Scheme 4.

### Example 1-159: 1-[2-(4-chlorophenyl)thiazol-4-ylmethyl]-5,5-dimethyl-3-(4 trifluoromethylsulfanylphenyl)imidazolidine-2,4-dione

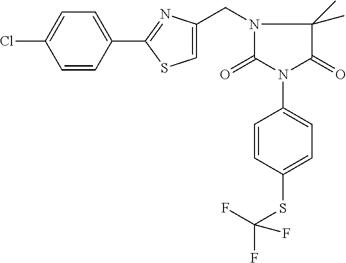 [SMILES: CC1(C)C(=O)N(c2ccc(SC(F)(F)F)cc2)C(=O)N1Cc1csc(-c2ccc(Cl)cc2)n1]

MS (LC-MS): 511.04; Retention time: 3.12.

The synthesis is described in Scheme 4.

### Example 1-160: 5,5-dimethyl-1-(1-methyl-1H-imidazol-2-ylmethyl)-3-(4-trifluoromethylsulfanylphenyl)imidazolidine-2,4-dione

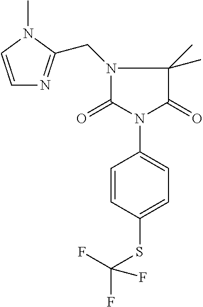 [SMILES: Cn1ccnc1CN1C(=O)N(c2ccc(SC(F)(F)F)cc2)C(=O)C1(C)C]

MS (LC-MS): 398.10; Retention time: 1.53.

The synthesis is described in Scheme 4.

### Example 1-161: 1-(7-methoxy-2-oxo-2H-chromen-4-ylmethyl)-5,5-dimethyl-3-(4-trifluoromethylsulfanylphenyl)imidazolidine-2,4-dione

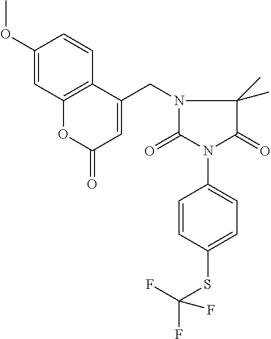 [SMILES: COc1ccc2c(CN3C(=O)N(c4ccc(SC(F)(F)F)cc4)C(=O)C3(C)C)cc(=O)oc2c1]

MS (LC-MS): 492.48; Retention time: 1.84.

The synthesis is described in Scheme 4.

### Example 1-162: 5,5-dimethyl-1-(5-methylestoxazol-3-ylmethyl)-3-(4-trifluoromethylsulfanylphenyl)imidazolidine-2,4-dione

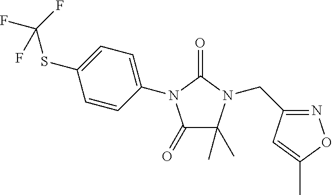 [SMILES: Cc1cc(CN2C(=O)N(c3ccc(SC(F)(F)F)cc3)C(=O)C2(C)C)no1]

MS (LC-MS): 500.09; Retention time: 2.52.

The synthesis is described in Scheme 4.

### Example 1-163: 1-[5-(4-methoxyphenyl)[1,2,4]oxadiazol-3-ylmethyl]-5,5-dimethyl-3-(4-trifluoromethylsulfanylphenyl)imidazolidine-2,4-dione

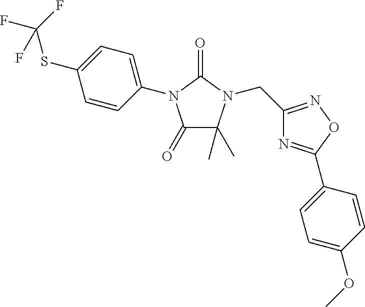 [SMILES: COc1ccc(-c2nc(CN3C(=O)N(c4ccc(SC(F)(F)F)cc4)C(=O)C3(C)C)no2)cc1]

MS (LC-MS): 492.11; Retention time: 1.87.

The synthesis is described in Scheme 4.

### Example 1-164: 1-[5-(4-methoxyphenyl)[1,2,4]oxadiazol-3-ylmethyl]-5,5-dimethyl-3-(4-trifluoromethoxyphenyl)imidazolidine-2,4-dione

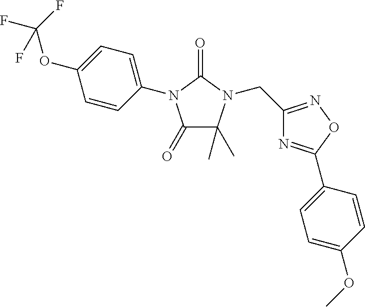 [SMILES: COc1ccc(-c2nc(CN3C(=O)N(c4ccc(OC(F)(F)F)cc4)C(=O)C3(C)C)no2)cc1]

MS (LC-MS): 476.13; Retention time: 2.03.

The synthesis is described in Scheme 4.

### Example 1-165: Methyl 5-[5,5-dimethyl-2,4-dioxo-3-(4-tri-fluoromethylsulfanylphenyl)imidazolidin-1-ylmethyl]-furan-2-carboxylate

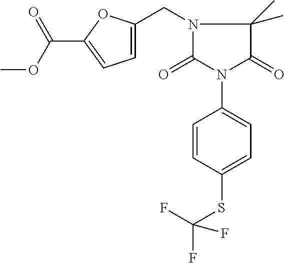

MS (LC-MS): 442.42; Retention time: 1.74.

The synthesis is described in Scheme 4.

### Example 1-166: 1-(1-benzyl-1H-imidazol-2-ylmethyl)-5,5-dimethyl-3-(4-trifluoromethylsulfanylphenyl)imidazolidine-2,4-dione

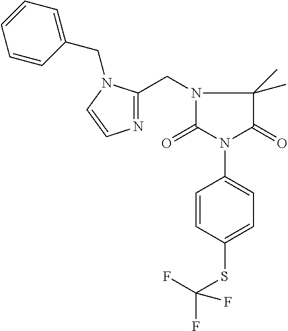 [SMILES: CC1(C)C(=O)N(c2ccc(SC(F)(F)F)cc2)C(=O)N1Cc1nccn1Cc1ccccc1]

MS (LC-MS): 474.13; Retention time: 2.02.

The synthesis is described in Scheme 4.

### Example 1-167: 1-(4-dimethylaminopyrimidin-2-ylmethyl)-5,5-dimethyl-3-(4-trifluoromethylsulfanylphenyl)imidazolidine-2,4-dione

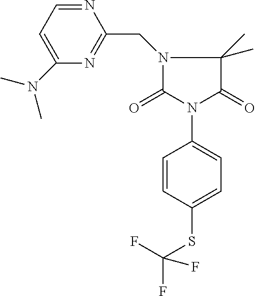

MS (LC-MS): 439.46; Retention time: 1.91.

The synthesis is described in Scheme 4.

### Example 1-168: 1-[1-(4-methoxybenzyl)-1H-imidazol-2-ylmethyl]-5,5-dimethyl-3-(4-trifluoromethylsulfanylphenyl)imidazolidine-2,4-dione

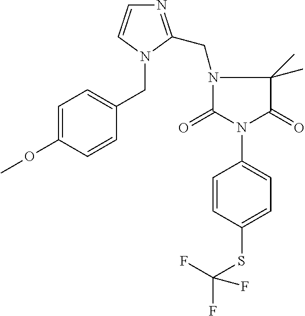

MS (LC-MS): 504.14; Retention time: 2.24.

The synthesis is described in Scheme 4.

### Example 1-169: 5,5-dimethyl-1-[2-(1-methylpyrrolidin-2-yl)-ethyl]-3-(4-trifluoromethylsulfanylphenyl)imidazolidine-2,4-dione

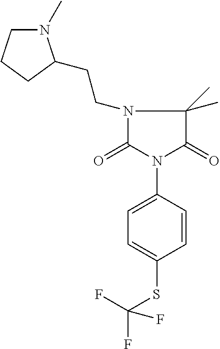 [SMILES: CN1CCCC1CCN1C(=O)N(c2ccc(SC(F)(F)F)cc2)C(=O)C1(C)C]

MS (LC-MS): 415.15; Retention time: 1.66 min.

The synthesis is described in Scheme 4.

### Example 1-170: 5,5-dimethyl-1-(2-morpholin-4-ylethyl)-3-(4-trifluoromethylsulfanylphenyl)imidazolidine-2,4-dione

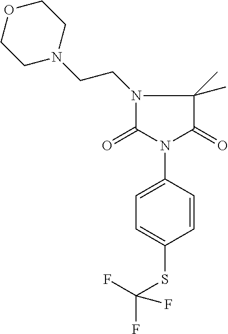 [SMILES: CC1(C)C(=O)N(c2ccc(SC(F)(F)F)cc2)C(=O)N1CCN1CCOCC1]

MS (LC-MS): 417.13; Retention time: 1.62 min.

The synthesis is described in Scheme 4.

### Example 1-171: 1-[5-(2-methoxyphenyl)[1,2,4]oxadiazol-3-ylmethyl]-5,5-dimethyl-3-(4-trifluoromethylsulfanylphenyl)imidazolidine-2,4-dione

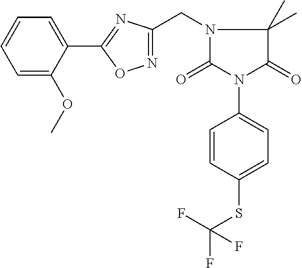 [SMILES: COc1ccccc1-c1nc(CN2C(=O)N(c3ccc(SC(F)(F)F)cc3)C(=O)C2(C)C)no1]

MS (LC-MS): 492.11; Retention time: 2.67 min.

The synthesis is described in Scheme 4.

### Example 1-172: 5,5-dimethyl-1-(5-methylestoxazol-3-ylmethyl)-3-(4-trifluoromethoxyphenyl)imidazolidine-2,4-dione

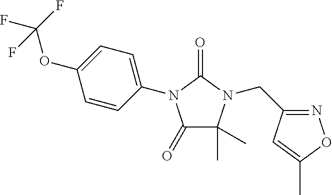 [SMILES: Cc1cc(CN2C(=O)N(c3ccc(OC(F)(F)F)cc3)C(=O)C2(C)C)no1]

MS (LC-MS): 383.33; Retention time: 1.95 min.

The synthesis is described in Scheme 4.

### Example 1-173: 1-(5-tert-butyl[1,2,4]oxadiazol-3-ylmethyl)-5,5-dimethyl-3-(4-trifluoromethylsulfanylphenyl)imidazolidine-2,4-dione

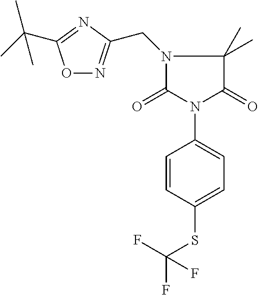 [SMILES: CC(C)(C)c1nc(CN2C(=O)N(c3ccc(SC(F)(F)F)cc3)C(=O)C2(C)C)no1]

MS (LC-MS): 442.16; Retention time: 2.64 min.

The synthesis is described in Scheme 4.

### Example 1-174: 4-[5,5-dimethyl-2,4-dioxo-3-(4-trifluoro-methylsulfanylphenyl)imidazolidin-1-ylmethyl]pyridine-2-carboxylic acid 4-methylbenzylamide with trifluoroacetic acid

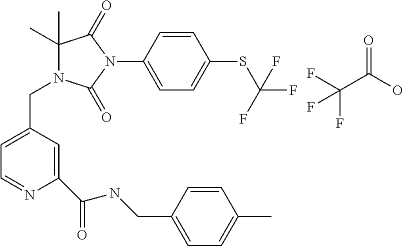

MS (LC-MS): 542.16; Retention time: 2.80 min.

The synthesis is described in Scheme 5.

### Example 1-175: 4-[5,5-dimethyl-2,4-dioxo-3-(4-trifluoro-methylsulfanylphenyl)imidazolidin-1-ylmethyl]pyridine-2-carboxylic acid (2-p-tolylethyl)amide

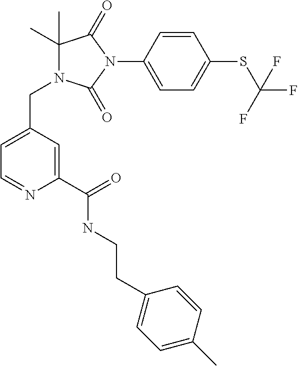 [SMILES: Cc1ccc(CCNC(=O)c2cc(CN3C(=O)N(c4ccc(SC(F)(F)F)cc4)C(=O)C3(C)C)ccn2)cc1]

MS (LC-MS): 556.18; Retention time: 2.31.

The synthesis is described in Scheme 5.

### Example 1-176: 4-[5,5-dimethyl-2,4-dioxo-3-(4-trifluoro-methylsulfanylphenyl)imidazolidin-1-ylmethyl]pyridine-2-carboxylic acid 3,4-dimethoxybenzylamide

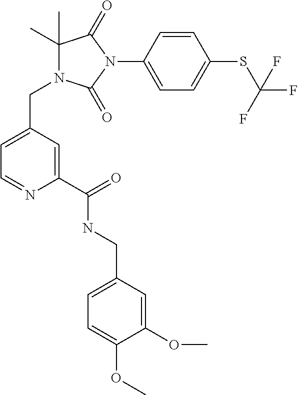 [SMILES: COc1ccc(CNC(=O)c2cc(CN3C(=O)N(c4ccc(SC(F)(F)F)cc4)C(=O)C3(C)C)ccn2)cc1OC]

MS (LC-MS): 588.16; Retention time: 1.58.

The synthesis is described in Scheme 5.

### Example 1-177: 4-[5,5-dimethyl-2,4-dioxo-3-(4-trifluoro-methylsulfanylphenyl)imidazolidin-1-ylmethyl]pyridine-2-carboxylic acid benzylamide

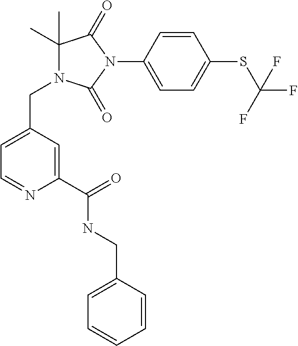 [SMILES: CC1(C)C(=O)N(c2ccc(SC(F)(F)F)cc2)C(=O)N1Cc1ccnc(C(=O)NCc2ccccc2)c1]

MS (LC-MS): 528.14; Retention time: 1.77.

The synthesis is described in Scheme 5.

### Example 1-178: 5,5-dimethyl-1-[2-(4-oxo-2-thioxo-1,4-dihydro-2H-quinazolin-3-yl)pyrid-4-ylmethyl]-3-(4-trifluoromethylsulfanylphenyl)imidazolidine-2,4-dione

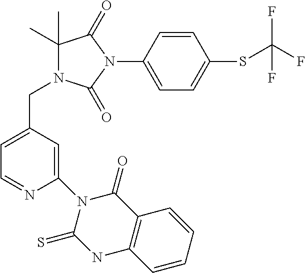 [SMILES: CC1(C)C(=O)N(c2ccc(SC(F)(F)F)cc2)C(=O)N1Cc1ccnc(-n2c(=S)[nH]c3ccccc3c2=O)c1]

MS (LC-MS): 571.10; Retention time: 2.3.

The synthesis is described in Scheme 6.

The two reaction schemes below describe the synthesis of compound of Formula (I) according to the present invention, especially among the products of Example 1-178 to Example 1-199 below.

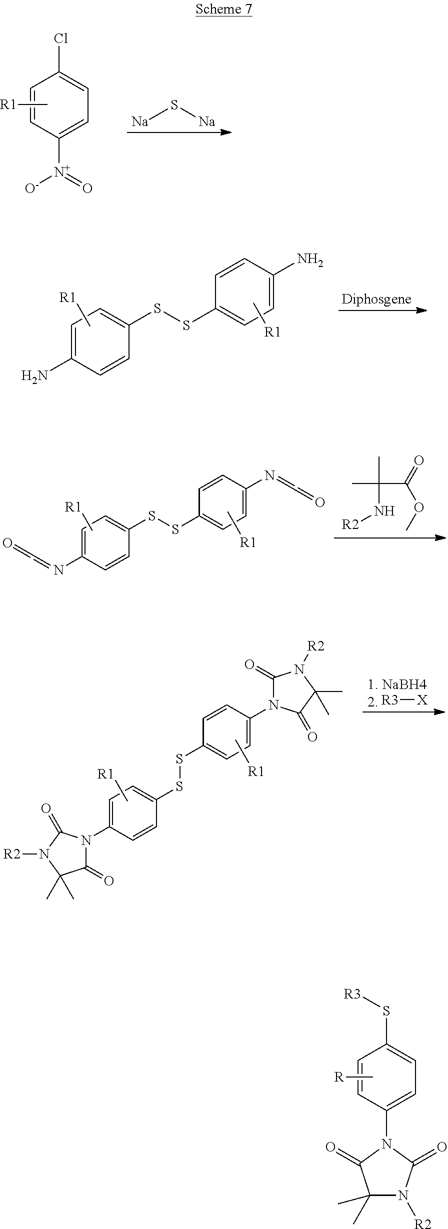

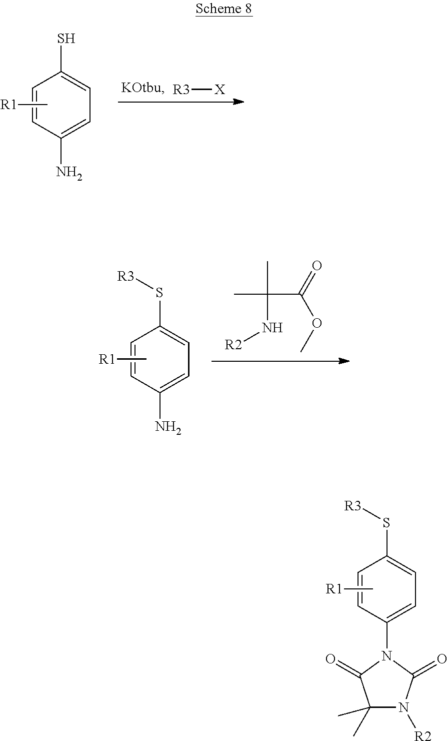

### Bis(4-amino-2-chlorophenyl) disulfide

76.8 g (0.4 mol) of 1,2-dichloro-4-nitrobenzene, suspended in 120 ml of water, were heated to 90° C. and treated with a solution of 62.4 g (0.8 mol) of sodium sulfide and 12.8 g (0.4 mol) of sulfur in 200 ml of water.

After refluxing the mixture for 5 hours, 6 g of carbon dioxide were bubbled through the solution, followed by a regular stream of air. The pH was adjusted to 5.5, the mixture was cooled to room temperature and the resulting precipitate was collected by filtration.

The crude material was recrystallized from isopropanol to give 45 g (71%) of the desired product.

M+H⁺=318.

LC/MS retention time=1.526.

### Bis[(2-chloro-4-(4,4-dimethyl-2, 5-dioxo-3-pyrid-4-ylmethylimidazolidin-1-yl)]phenyl disulfide

A solution of 13.7 g (69.5 mmol) of diphosgene in 100 ml of toluene was cooled to −20° C. and treated with a solution of 5.0 g (15.8 mmol) of bis(4-amino-2-chlorophenyl) disulfide. The resulting mixture was stirred for 30 minutes at room temperature, refluxed for 1 hour and then evaporated to dryness.

2.53 g (about 6.86 mmol) of this material were dissolved in THF and treated with 2.0 g (9.5 mmol) of methyl 2-methyl-2-[(pyrid-4-ylmethyl)amino]propionate. The mixture was stirred for 3 hours at room temperature and evaporated to dryness, and the resulting solid was purified by flash chromatography (SiO₂, methylene chloride:methanol=97:3) to give 1.8 g (37%) of the desired product.

M+H⁺=722.

LC/MS retention time=1.176.

### Example 1-179: 3-(4-tert-butylsulfanyl-3-chlorophenyl)-5,5-dimethyl-1-pyrid-4-yl-methylimidazolidine-2,4-dione trifluoroacetate

200 mg (0.28 mol) of bis[(2-chloro-4-(4,4-dimethyl-2,5-dioxo-3-pyrid-4-yl-methylimidazolidin-1-yl)]phenyl disulfide were dissolved in 10 ml of methanol and treated with 22 mg (0.56 mmol) of sodium borohydride. After stirring for 30 minutes at room temperature, the mixture was evaporated to dryness. The residue was dissolved in 10 ml of sulfuric acid (75%) and added to 20 ml of sulfuric acid (75%) saturated with isobutylene. The mixture was stirred for 20 minutes at room temperature and then added cautiously to a cooled solution of sodium hydroxide in water. The alkaline aqueous phase was extracted three times with ethyl acetate, the combined organic phases were dried with sodium sulfate and the material remaining after evaporation was filtered off on silica gel (methylene chloride/methanol=95/5). The resulting crude product was purified by preparative HPLC chromatography (RP 18, acetonitrile, water, 0.01% TFA) to give 120 mg (81%) of the desired product.

M+H⁺=418.

LC/MS retention time=1.249.

### General Procedure 1A: Preparation of 3-(3-chloro-4-alkylsulfanylphenyl)-5,5-dimethyl-1-pyrid-4-ylmethylimidazolidine-2,4-diones

445 mg (0.62 mol) of bis[(2-chloro-4-(4,4-dimethyl-2,5-dioxo-3-pyrid-4-yl-methylimidazolidin-1-yl)]phenyl disulfide were dissolved in 15 ml of methanol and treated with 49 mg (1.24 mmol) of sodium borohydride. After stirring for 30 minutes at room temperature, 1.24 mmol of the respective alkyl halide were added and the resulting mixture was refluxed for 1 hour. The solvent was removed by evaporation and the remaining crude materials were purified by preparative HPLC chromatography (RP 18, acetonitrile, water, 0.01% TFA).

### Example 1-180: 3-(3-chloro-4-isopropylsulfanylphenyl)-5,5-dimethyl-1-pyrid-4-yl-methylimidazolidine-2,4-dione

Synthesis according to the general procedure 1A.

M+H⁺=404.

LC/MS retention time=1.196.

### Example 1-181: 3-(3-chloro-4-isobutylsulfanylphenyl)-5,5-dimethyl-1-pyrid-4-yl-methylimidazolidine-2,4-dione trifluoroacetate

Prepared according to the general procedure 1A.

M+H⁺=418.

LC/MS retention time=1.272.

### Example 1-182: 3-(3-chloro-4-methylsulfanylphenyl)-5,5-dimethyl-1-pyrid-4-yl-methylimidazolidine-2,4-dione

Prepared according to the general procedure 1A.

M+H⁺=376.

LC/MS retention time=1.054.

### Example 1-183: 3-[3-chloro-4-(3-methoxypropylsulfanyl)phenyl]-5,5-dimethyl-1-pyrid-4-ylmethylimidazolidine-2,4-dione trifluoroacetate

Prepared according to the general procedure 1A.

M+H⁺=434.

LC/MS retention time=1.096.

### Example 1-184: 3-[3-chloro-4-(2-morpholin-4-ylethylsulfanyl)phenyl]-5,5-dimethyl-1-pyrid-4-ylmethylimidazolidine-2,4-dione trifluoroacetate

Prepared according to the general procedure 1A.

M+H⁺=475.

LC/MS retention time=0.792

### Example 1-185: 3-{3-chloro-4-[2-(1-methylpyrrolidin-2-yl)ethylsulfanyl]phenyl}-5,5-dimethyl-1-pyrid-4-ylmethylimidazolidine-2,4-dione trifluoroacetate

Prepared according to the general procedure 1A.

M+H⁺=473.

LC/MS retention time=0.865.

### Example 1-186: 3-{3-chloro-4-[3-(4-methylpiperazin-1-yl)propylsulfanyl]phenyl}-5,5-dimethyl-1-pyrid-4-ylmethylimidazolidine-2,4-dione trifluoroacetate

Prepared according to the general procedure 1A.

M+H⁺=502.

LC/MS retention time=0.795.

### Example 1-187: 3-[3-chloro-4-(3-hydroxypropylsulfanyl)phenyl]-5,5-dimethyl-1-pyrid-4-ylmethylimidazolidine-2,4-dione trifluoroacetate

Prepared according to the general procedure 1A.

M+H⁺=420.

LC/MS retention time=0.968.

### Example 1-188: 3-[3-chloro-4-(2-hydroxyethylsulfanyl)phenyl]-5,5-dimethyl-1-pyrid-4-ylmethylimidazolidine-2,4-dione trifluoroacetate

Prepared according to the general procedure 1A.

M+H⁺=406.

LC/MS retention time=0.921.

### Example 1-189: [2-chloro-4-(4,4-dimethyl-2,5-dioxo-3-pyrid-4-ylmethylimidazolidin-1-yl)phenylsulfanyl]acetonitrile trifluoroacetate

Prepared according to the general procedure 1A.

M+H⁺=401.

LC/MS retention time=0.999.

### Example 1-190: 3-nitro-4-trifluoromethoxyaniline

20 g (112.9 mmol) of 4-trifluoromethoxyaniline were dissolved in 50 ml of concentrated sulfuric acid at a temperature of 0 to 5° C., treated with 30 ml of a 4/1 mixture of sulfuric acid and nitric acid. The mixture was stirred for 5 hours at 0° C. and then poured into an ice-cold water mixture and made alkaline with 200 ml of concentrated aqueous ammonia solution. Extraction with EE, drying with sodium sulfate, evaporation to dryness and recrystallization from ethyl acetate/heptane gave 15.5 g (63%) of the desired product.

M+H⁺=223.

LC/MS retention time=1.378.

### Example 1-191: Trifluoroacetic acid; 5,5-dimethyl-3-(3-nitro-4-trifluoro-methoxyphenyl)-1-pyrid-4-ylmethylimidazolidine-2,4-dione

2.27 g (11.5 mmol) of diphosgene in 1,2-dichloroethane were added at −20° C. to 1.0 g (4.5 mmol) of 3-nitro-4-trifluoromethoxyaniline, dissolved in 20 ml of 1,2-dichloroethane. The mixture was stirred for 1 hour while warming to room temperature and was then heated at 50° C. for 2 hours. After leaving to stand overnight, the solvent was evaporated off and the residue was taken up in dry THF and treated with 937 mg (4.5 mmol) of methyl 2-methyl-2-[(pyrid-4-ylmethyl)amino]propionate. The mixture was stirred for 2 hours at room temperature and for 1 hour at 40° C., and then evaporated to dryness and the remaining residue was purified (RP 18, acetonitrile, water, 0.01% TFA) to give 1.15 g (61%) of the desired product.

M+H⁺=425.

LC/MS retention time=1.324.

### Example 1-192: 3-(3-amino-4-trifluoro-methoxyphenyl)-5,5-dimethyl-1-pyrid-4-yl-methylimidazolidine-2,4-dione

9.63 g (22.7 mmol) of 5,5-dimethyl-3-(3-nitro-4-trifluoromethoxyphenyl)-1-pyrid-4-ylmethylimidazolidine-2,4-dione trifluoroacetate were dissolved in semi-concentrated hydrochloric acid, heated to reflux and treated with 30 g of zinc powder. After 1.5 hours, the mixture was cooled, filtered and extracted twice with methyl tert-butyl ether. The aqueous phase was made alkaline with 6 N sodium hydroxide and extracted with methyl tert-butyl ether, and the combined organic phases were dried over sodium sulfate. Evaporation of the solvent gave 5.0 g (56%) of the desired product.

M+H⁺=395.

LC/MS retention time=0.948.

### Example 1-193: 5,5-dimethyl-1-pyrid-4-ylmethyl-3-[4-(2,2,2-trifluoroethoxy)phenyl]imidazolidine-2,4-dione

688 mg (3.6 mmol) of 2,2,2-trifluoroethoxyaniline in 30 ml of methylene chloride were added at 0° C. to 356 mg (3.6 mmol) of triethylamine and a solution of 356 mg (1.2 mmol) of triphosgene in 30 ml of methylene chloride. The mixture was stirred overnight while warming to room temperature. Next, the solvent was evaporated off, the residue was taken up in 15 ml of THF and treated with 100 mg (0.48 mmol) of methyl 2-methyl-2-[(pyrid-4-ylmethyl)amino]propionate and 48.5 mg (0.48 mmol) of triethylamine. The mixture was stirred for 4 hours at room temperature and for 1 hour at 50° C., and then evaporated to dryness, and the remaining residue was purified by preparative HPLC chromatography (RP 18, acetonitrile, water, 0.01% TFA) to give 67 g (36%) of the desired product.

M+H⁺=394.

LC/MS retention time=1.032.

### Example 1-194: 3-(8-chloro-3,4,4-trimethylthiochroman-6-yl)-5,5-dimethyl-1-pyrid-4-ylmethylimidazolidine-2,4-dione

200 mg (0.28 mol) of bis[(2-chloro-4-(4,4-dimethyl-2,5-dioxo-3-pyrid-4-yl-methylimidazolidin-1-yl)]phenyl disulfide were dissolved in 10 ml of methanol and treated with 22 mg (0.56 mmol) of sodium borohydride. After stirring for 30 minutes at room temperature, the mixture was evaporated to dryness. The residue was dissolved in 10 ml of sulfuric acid (75%) and added to 20 ml of sulfuric acid (75%) saturated with isobutylene. The mixture was stirred for 30 minutes at 40° C., while a regular airstream of isobutylene was bubbled through the solution. After leaving to stand overnight, the mixture was added cautiously to a cooled solution of sodium hydroxide in water. The alkaline aqueous phase was extracted three times with ethyl acetate, the combined organic phases were dried over sodium sulfate and the material remaining after evaporation was filtered on silica gel (methylene chloride/methanol=95/5). The resulting crude product was purified by preparative HPLC chromatography (RP 18, acetonitrile, water, 0.01% TFA) to give 70 mg (56%) of the desired product.

M+H⁺=444.

LC/MS retention time=1.240.

### Example 1-195: 1-(2-chlorothiazol-5-ylmethyl)-5,5-dimethyl-3-(4-trifluoro-methylsulfanylphenyl)imidazolidine-2,4-dione trifluoroacetate

200 mg (0.65 mmol) of 5,5-dimethyl-3-(4-trifluoro-methylsulfanylphenyl)-imidazolidine-2,4-dione, 115 mg (0.82 mmol) of potassium carbonate and 276 mg (1.64 mmol) of 2-chloro-5-chloromethylthiazole were dissolved in 2 ml of DMF and stirred at room temperature for 2 days. The mixture was poured into water, extracted three times with ethyl acetate, the combined organic phases were dried over sodium sulfate and the material remaining after evaporation was purified by preparative HPLC chromatography (RP 18, acetonitrile, water, 0.01% TFA) to give 143 mg (50%) of the desired product.

M+H⁺=436.

LC/MS retention time=1.784.

### Example 1-196: 3-[3-chloro-4-(propane-2-sulfonyl)phenyl]-5,5-dimethyl-1-pyrid-4-ylmethylimidazolidine-2,4-dione

40 mg (0.1 mmol) of 3-(3-chloro-4-isopropylsulfanylphenyl)-5,5-dimethyl-1-pyrid-4-ylmethylimidazolidine-2,4-dione were dissolved in 2 ml of methylene chloride and treated with a solution of 37 mg (0.15 mmol) of m-chloroperbenzoic acid in 2 ml of methylene chloride. The reaction was monitored by TLC and stopped when the sulfoxide and the sulfone were formed in equal amounts. The mixture was evaporated to dryness and purified by flash chromatography (SiO₂, methylene chloride/methanol=98/2) to give 4 mg of 3-[3-chloro-4-(propane-2-sulfonyl)phenyl]-5,5-dimethyl-1-pyrid-4-ylmethylimidazolidine-2,4-dione and 7 mg of 3-[3-chloro-4-(propane-2-sulfinyl)phenyl]-5,5-dimethyl-1-pyrid-4-ylmethylimidazolidine-2,4-dione.

M+H⁺=436.

LC/MS retention time=0.970.

### Example 1-197: 3-[3-chloro-4-(propane-2-sulfinyl)phenyl]-5,5-dimethyl-1-pyrid-4-ylmethylimidazolidine-2,4-dione

Prepared as described above.

M+H⁺=420.

LC/MS retention time=0.932.

### 2-(Isoquinolin-5-ylamino)-2-methylpropionitrile

5.0 g (34.7 mmol) of 5-aminoisoquinoline, 5.1 ml (69.4 mmol) of acetone and 945 mg (6.94 mmol) of zinc chloride were dissolved in 100 ml of acetonitrile and treated at 0° C. with 6.9 g (69.4 mmol) of trimethylsilyl cyanide. The mixture was refluxed for 3 hours, the solvent was then evaporated off and the residue was taken up in 200 ml of sodium hydrogen carbonate and extracted three times with ethyl acetate. The combined organic phases were dried and the residue remaining after evaporation was purified by flash chromatography (SiO₂, methylene chloride/methanol=95/5) to give 6.0 g (82%) of the desired product.

M+H⁺=212.

LC/MS retention time=0.696.

### General Procedure 2: Synthesis of Alkylsulfanylanilines

1.1 g (8 mmol) of 4-aminothiophenol and 896 mg (8 mmol) of potassium tert-butoxide were dissolved in 10 ml of DMF and stirred for 45 minutes under an argon atmosphere. Next, 8.8 mmol of the corresponding alkyl bromide were added and the mixture was stirred for 3 hours at room temperature, poured into water and extracted with ethyl acetate. The combined organic phases were dried and evaporated. The residual product was essentially pure and could be used without further purification.

### 4-(3-Methoxypropylsulfanyl)phenylamine

Prepared according to the general procedure 2.

M+H⁺=197.

LC/MS retention time=0.777.

### 4-Isopropylsulfanylphenylamine

Prepared according to the general procedure 2.

M+H⁺=266.

LC/MS retention time=0.173.

### 4-[3-(4-Methylpiperazin-1-yl)propylsulfanyl]phenylamine

Prepared according to the general procedure 2.

M+H⁺=168.

LC/MS retention time=0.894.

### General procedure 3: Preparation of 3-(3-chloro-4-alkylsulfanylphenyl)-5,5-dimethyl-1-pyrid-4-ylmethylimidazolidine-2,4-diones

371 mg (2.3 mmol) of carbonyldiimidazole, 41 mg (0.5 mmol) of imidazole and 1.9 mmol of the respective alkylsulfanylaniline were dissolved at 0° C. in 10 ml of THF and stirred for 1 hour. Next, 280 mg (1.35 mmol) of methyl 2-methyl-2-[(pyrid-4-yl-methyl)amino]propionate dissolved in 5 ml of THF were added and the mixture was refluxed for 5 hours. Next, the solvent was evaporated off and the material remaining after evaporation was purified by preparative HPLC chromatography (RP 18, acetonitrile, water, 0.01% TFA).

### Preparation of 5,5-dimethyl-3-(4-methyl sulfanylphenyl)-1-pyrid-4-ylmethylimidazolidine-2,4-dione trifluoroacetate

Prepared according to the general procedure 3.

M+H⁺=342.

LC/MS retention time=0.942.

### Example 1-198: 3-[4-(3-methoxypropylsulfanyl)phenyl]-5,5-dimethyl-1-pyrid-4-yl-methylimidazolidine-2,4-dione trifluoroacetate

Prepared according to the general procedure 3.

M+H⁺=400.

LC/MS retention time=1.007.

### Example 1-199: 3-(4-isopropylsulfanylphenyl)-5,5-dimethyl-1-pyrid-4-yl-methylimidazolidine-2,4-dione trifluoroacetate

Prepared according to the general procedure 3.

M+H⁺=370.

LC/MS retention time=1.096.

### Example 1-200: 5,5-dimethyl-3-{4-[3-(4-methylpiperazin-1-yl)propylsulfanyl]phenyl}-1-pyrid-4-ylmethylimidazolidine-2,4-dione trifluoroacetate

Prepared according to the general procedure 3.

M+H⁺=468.

LC/MS retention time=0.689

### Example 1-201: 5,5-Dimethyl-1-(6-methoxyquinolin-4-ylmethyl)-3-(4-trifluoro-methoxyphenyl)imidazolidine-2,4-dione

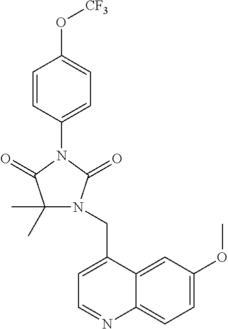 [SMILES: COc1ccc2nccc(CN3C(=O)N(c4ccc(OC(F)(F)F)cc4)C(=O)C3(C)C)c2c1]

0.1 g of sodium hydride (at 60%) is added to a solution of 0.65 g of 5,5-dimethyl-3-(4-trifluoromethoxyphenyl)imidazolidine-2,4-dione in 40 ml of anhydrous THF, under an inert atmosphere of argon at a temperature in the region of 20° C. Stirring is maintained at this temperature for 30 minutes. 0.47 g of 4-chloromethyl-6-methoxyquinoline dissolved in 10 ml of THF is added. The reaction medium is refluxed for 16 hours. After cooling, 100 ml of water and 75 ml of ethyl acetate are added. After separation of the phases by settling, the organic phase is dried over sodium sulfate, filtered and then concentrated under reduced pressure. The brown oil obtained is purified by flash chromatography (SiO₂, EtOAc/cyclohexane, 50/50 by volume, as eluent, Ar). The fractions containing the product are concentrated under reduced pressure. 0.36 g of 5,5-dimethyl-1-(6-methoxyquinolin-4-ylmethyl)-3-(4-trifluoromethoxyphenyl)imidazolidine-2,4-dione is thus obtained, the characteristics of which product are as follows:

¹H NMR spectrum (300 MHz, (CD₃)₂SO d6, δ in ppm): 1.45 (s: 6H); 3.96 (s: 3H); 5.10 (broad s: 2H); 7.46 (dd, J=9 and 3 Hz: 1H); 7.54 (d, J=3 Hz: 1H); 7.56 (broad d, J=9 Hz: 2H); 7.61 (d, J=4.5 Hz: 1H); 7.68 (broad d, J=9 Hz: 2H); 8.00 (d, J=9 Hz: 1H); 8.72 (d, J=4.5 Hz: 1H).

Mass IE m/z=459 M+. base peak

m/z=213 C₁₂H₉N₂O₂⁺

m/z=172 C₁₁H₁₀NO⁺

The compound 5,5-dimethyl-3-(4-trifluoromethoxyphenyl)imidazolidine-2,4-dione is prepared according to the process described in Example 1-63.

### Preparation of 4-chloromethyl-6-methoxyquinoline

1.16 ml of triethylamine and 0.64 ml of methanesulfonyl chloride are successively added to a solution of 1.2 g of 4-hydroxymethyl-6-methoxyquinoline in 45 ml of dichloromethane, under an inert atmosphere of argon at a temperature in the region of 20° C. Stirring is maintained at this temperature for 3 hours. The reaction medium is concentrated under reduced pressure to give a brown residue. The product is used without further purification in the following step.

¹H NMR spectrum (300 MHz, (CD₃)₂SO d6, δ in ppm): 3.97 (s: 3H); 5.30 (s: 2H); 7.48 (dd, J=9 and 3 Hz: 1H); 7.52 (d, J=3 Hz: 1H); 7.62 (d, J=4.5 Hz: 1H); 8.00 (d, J=9 Hz: 1H); 8.75 (d, J=4.5 Hz: 1H).

Mass IE m/z=207 M⁺.

m/z=172 (M-Cl)⁺ base peak

m/z=157 (m/z=172-CH₃)⁺

m/z=129 (m/z=157-CO)⁺

### Preparation of 4-hydroxymethyl-6-methoxyquinoline

17 ml of a 1M solution of LiAlH₄ in THF are added dropwise to a solution of 4 g of 4-thoxycarbonyl-6-methoxyquinoline in 100 ml of anhydrous THF, under an inert atmosphere of argon at a temperature in the region of 5° C. The reaction medium is maintained with stirring for 16 hours at a temperature in the region of 20° C. 50 ml of water and 50 ml of ethyl acetate are added. After separation of the phases by settling, the organic phase is dried over sodium sulfate, filtered and then concentrated under reduced pressure. The foam obtained is purified by flash chromatography (SiO₂, EtOAc as eluent, Ar). The fractions containing the product are concentrated under reduced pressure. 0.86 g of 4-hydroxymethyl-6-methoxyquinoline is thus obtained, the characteristics of which product are as follows:

¹H NMR spectrum (300 MHz, (CD₃)₂SO d6, δ in ppm): 3.93 (s: 3H); 5.02 (d, J=5.5 Hz: 2H); 5.57 (t, J=5.5 Hz: 1H); 7.30 (d, J=3 Hz: 1H); 7.38 (dd, J=9 and 3 Hz: 1H); 7.50 (d, J=4.5 Hz: 1H); 7.92 (d, J=9 Hz: 1H); 8.69 (d, J=4.5 Hz: 1H).

Mass IE, m/z=189 M⁺. base peak

m/z=174 (M-CH₃)⁺

m/z=160 (M-CHO)⁺

m/z=146 (m/z=174-CO)⁺

m/z=117 (m/z=146-CHO)⁺.

### Example 1-202: 5,5-Dimethyl-1-(6-hydroxyquinolin-4-ylmethyl)-3-(4-trifluoro-methoxyphenyl)imidazolidine-2,4-dione

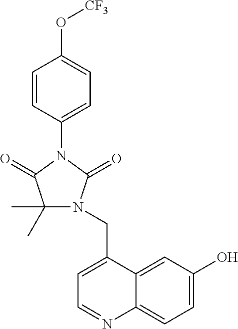 [SMILES: CC1(C)C(=O)N(c2ccc(OC(F)(F)F)cc2)C(=O)N1Cc1ccnc2ccc(O)cc12]

6 ml of a 1M solution of boron tribromide in CH₂Cl₂ are added to a solution of 0.26 g of 5,5-dimethyl-1-(6-methoxyquinolin-4-ylmethyl)-3-(4-trifluoromethoxyphenyl)imidazolidine-2,4-dione in 40 ml of dichloromethane, under an inert atmosphere of argon at a temperature in the region of 0° C. The reaction medium is stirred at a temperature in the region of 20° C. for 16 hours. 5 ml of methanol are added dropwise. After stirring for 30 minutes at this temperature, 50 ml of water, 30 ml of CH₂Cl₂ and 10 ml of saturated NaHCO₃ are added. After separation of the phases by settling, the organic phase is dried over sodium sulfate, filtered and then concentrated under reduced pressure. The beige-colored foam obtained is purified by flash chromatography (SiO₂, EtOAc as eluent, Ar). The fractions containing the product are concentrated under reduced pressure. 0.17 g of 5,5-dimethyl-1-(6-hydroxyquinolin-4-ylmethyl)-3-(4-trifluoromethoxyphenyl)imidazolidine-2,4-dione is thus obtained, the characteristics of which product are as follows:

¹H NMR spectrum (300 MHz, (CD₃)₂SO d6, δ in ppm): 1.46 (s: 6H); 5.00 (broad s: 2H); 7.30 to 7.40 (mt: 2H); 7.51 (d, J=4.5 Hz: 1H); 7.55 (broad d, J=8.5 Hz: 2H); 7.69 (d, J=8.5 Hz: 2H); 7.94 (d, J=9 Hz: 1H); 8.64 (d, J=4.5 Hz: 1H); 10.12 (unresolved complex: 1H).

Mass IE m/z=445 M⁺.

m/z=199 C₁₁H₇N₂O₂⁺

m/z=158 C₁₀H₈NO⁺ base peak

### Example 1-203: 5,5-Dimethyl-1-(7-methoxyquinolin-4-ylmethyl)-3-(4-trifluoro-methoxyphenyl)imidazolidine-2,4-dione

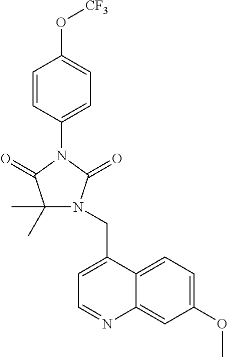 [SMILES: COc1ccc2c(CN3C(=O)N(c4ccc(OC(F)(F)F)cc4)C(=O)C3(C)C)ccnc2c1]

The product is prepared according to the procedure described in Example 59, starting with 0.94 g of methyl 2-methyl-2-[(7-methoxyquinolin-4-ylmethyl)amino]propanoate instead of methyl 2-methyl-2-[(quinolin-4-ylmethyl)amino]propanoate used in Example 1-59, and 1.7 g of 4-(trifluoromethoxyphenyl) isocyanate. After purification by flash chromatography on a column (SiO₂, cyclohexane/ethyl acetate 70/30 by volume and then CH2Cl2/MeOH 90/10 by volume as eluents, Ar), 1.45 g of the expected product are obtained.

¹H NMR spectrum (300 MHz, (CD₃)₂SO d6, δ in ppm): 1.44 (s: 6H); 3.96 (s: 3H); 5.12 (broad s: 2H); 7.35 (dd, J=9 and 3 Hz: 1H); 7.47 (d, J=3 Hz: 1H); 7.49 (d, J=4.5 Hz: 1H); 7.55 (broad d, J=9 Hz: 2H); 7.68 (broad d, J=9 Hz: 2H); 8.19 (d, J=9 Hz: 1H); 8.80 (d, J=4.5 Hz: 1H).

Mass IE m/z=459 M⁺. base peak

m/z=444 (M-CH₃)⁺

m/z=213 C₁₂H₉N₂O₂⁺

m/z=172 C₁₁H₁₀NO⁺

### Preparation of methyl 2-((7-methoxyquinolin-4-ylmethyl)amino)propanoate

A mixture of 1.23 g of methyl ca-aminoisobutyrate hydrochloride and 1.12 ml of triethylamine in 30 ml of dichloromethane is stirred at 0° C. for 20 minutes. Next, 1 g of magnesium sulfate and 1.5 g of 7-methoxyquinoline-4-carbaldehyde are added. Stirring is continued for 15 hours at room temperature and the mixture is then concentrated under reduced pressure. The residue is taken up in 35 ml of methanol, the solution obtained is cooled to 0° C. and 0.31 g of sodium borohydride is then added portionwise. The reaction medium is stirred at a temperature in the region of 20° C. for 15 hours. The reaction medium is concentrated under reduced pressure. The residue obtained is taken up in 100 ml of EtOAc. The precipitate formed is filtered off and the filtrate is concentrated under reduced pressure and then purified by flash chromatography on a column (SiO₂, EtOAc as eluent, Ar), and 0.95 g of the expected product is obtained.

¹H NMR spectrum (300 MHz, (CD₃)₂SO d6, δ in ppm): 1.35 (s: 6H); 2.63 (t, J=7.5 Hz: 1H); 3.70 (s: 3H); 3.93 (s: 3H); 4.05 (d, J=7.5 Hz: 2H); 7.28 (dd, J=9 and 3 Hz: 1H); 7.41 (d, J=3 Hz: 1H); 7.43 (d, J=4.5 Hz: 1H); 8.12 (d, J=9 Hz: 1H); 8.76 (d, J=4.5 Hz: 1H).

Mass IC m/z=289 MH⁺ base peak

m/z=229 (M-C₂H₄O₂)⁺

### Preparation of 7-methoxyquinoline-4-carbaldehyde

A mixture of 1.9 g of selenium oxide dissolved in 35 ml of dioxane is added dropwise to a solution of 2.7 g of 4-methyl-7-methoxyquinoline in 35 ml of dioxane preheated to 65° C. At the end of the addition, the brown suspension is heated to a temperature in the region of 80° C. for 5 hours. The reaction medium is stirred for 16 hours at a temperature in the region of 20° C. The greenish suspension is suction-filtered and then washed with EtOAc. The filtrate concentrated under reduced pressure. The suspension obtained is crystallized from isopropyl ether to give 1.54 g of 7-methoxyquinoline-4-carbaldehyde.

¹H NMR spectrum (300 MHz, (CD₃)₂SO d6, δ in ppm): 3.99 (s: 3H); 7.48 (dd, J=9 and 3 Hz: 1H); 7.57 (d, J=3 Hz: 1H); 7.90 (d, J=4.5 Hz: 1H); 8.89 (d, J=9 Hz: 1H); 9.19 (d, J=4.5 Hz: 1H); 10.52 (s: 1H).

Mass IC m/z=188 MH⁺ base peak

### Preparation of 4-methyl-7-methoxyquinoline

4 g of triphenylphosphine, 5.3 g of lithium chloride, 14 ml of tetramethyltin and 2.1 g of bis(triphenylphosphine)palladium(II) chloride are added to a solution of 6 g of 4-bromo-7-methoxyquinoline in 100 ml of DMF, under an inert atmosphere of argon at a temperature in the region of 20° C. The reaction medium is heated at a temperature in the region of 120° C. for 16 hours. After cooling, the insoluble material is filtered off. The filtrate is concentrated under reduced pressure. The residue obtained is taken up in 300 ml of EtOAc and 300 ml of water. After separation of the phases by settling, the organic phase is dried over magnesium sulfate, filtered and then concentrated under reduced pressure. The oil obtained is taken up in 300 ml of EtOAc and 300 ml of water and then acidified with hydrochloric acid to pH 1. The aqueous phase is basified with sodium hydroxide to pH 10 and then extracted with 300 ml of EtOAc. After separation of the phases by settling, the organic phase is dried over magnesium sulfate, filtered and then concentrated under reduced pressure to give 2.7 g of 4-methyl-7-methoxyquinoline, the characteristics of which product are as follows:

¹H NMR spectrum (300 MHz, (CD₃)₂SO d6, δ in ppm): 2.65 (s: 3H); 3.94 (s: 3H); 7.23 (broad d, J=4.5 Hz: 1H); 7.28 (dd, J=9 and 3 Hz: 1H); 7.40 (d, J=3 Hz: 1H); 8.01 (d, J=9 Hz: 1H); 8.58 (d, J=4.5 Hz: 1H).

Mass IE m/z=173 M⁺. base peak

m/z=158 (M-CH₃)⁺

m/z=143 (M-CH₂O)⁺.

m/z=130 (m/z=158-CO)⁺

### Preparation of 4-bromo-7-methoxyquinoline

22.74 g of 4-hydroxy-7-methoxyquinoline are added to 200 g of phosphorus oxybromide preheated to a temperature in the region of 110° C. The reaction medium is heated at this same temperature for 3 hours. The reaction medium is poured, while hot, into a mixture of 500 ml of EtOAc and 500 ml of ice-cold water. The medium is neutralized with potassium carbonate to pH 7. After separation of the phases by settling, the organic phase is dried over magnesium sulfate, evaporated under reduced pressure and then purified by column chromatography (SiO₂, EtOAc/cyclohexane 50/50 by volume as eluents, Ar), and 14.6 g of the expected product are obtained.

¹H NMR spectrum (300 MHz, (CD₃)₂SO d6, δ in ppm): 3.97 (s: 3H); 7.43 (dd, J=9 and 3 Hz: 1H); 7.49 (d, J=3 Hz: 1H); 7.79 (d, J=4.5 Hz: 1H); 8.06 (d, J=9 Hz: 1H); 8.67 (d, J=4.5 Hz: 1H).

Mass IE m/z=237 M⁺. base peak

4-Hydroxy-7-methoxyquinoline is prepared according to the process described in: J. Am. Chem. Soc., 68, 1268, 1946.

### Example 1-204: 5,5-Dimethyl-1-(7-hydroxyquinolin-4-ylmethyl)-3-(4-trifluoro-methoxyphenyl)imidazolidine-2,4-dione

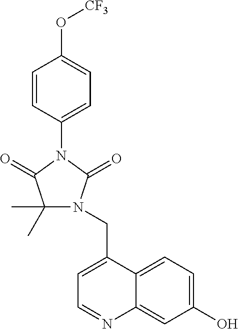 [SMILES: CC1(C)C(=O)N(c2ccc(OC(F)(F)F)cc2)C(=O)N1Cc1ccnc2cc(O)ccc12]

A mixture of 0.89 g of 5,5-dimethyl-1-(7-methoxyquinolin-4-ylmethyl)-3-(4-trifluoro-methoxyphenyl)imidazolidine-2,4-dione and 5.5 g of pyridine hydrochloride is heated at a temperature in the region of 220° C. for 4 hours. After cooling, 200 ml of water and 100 ml of CH₂Cl₂ are added. After separation of the phases by settling, the organic phase is dried over sodium sulfate, filtered and then concentrated under reduced pressure. The solid obtained is purified by flash chromatography (SiO₂, EtOAc/CH₂Cl₂ 40/60 by volume as eluent, Ar). The fractions containing the product are concentrated under reduced pressure. 85 mg of 5,5-dimethyl-1-(7-hydroxyquinolin-4-ylmethyl)-3-(4-trifluoromethoxyphenyl)imidazolidine-2,4-dione are thus obtained, the characteristics of which product are as follows:

¹H NMR spectrum (300 MHz, (CD₃)₂SO d6, δ in ppm): 1.45 (s: 6H); 5.08 (broad s: 2H); 7.24 (dd, J=9 and 3 Hz: 1H); 7.30 (d, J=3 Hz: 1H); 7.38 (d, J=4.5 Hz: 1H); 7.55 (broad d, J=9 Hz: 2H); 7.68 (broad d, J=9 Hz: 2H); 8.10 (d, J=9 Hz: 1H); 8.71 (d, J=4.5 Hz: 1H); from 9.90 to 10.50 (broad unresolved complex: 1H).

Mass IE m/z=445 M⁺. base peak

m/z=430 (M-CH₃)⁺

m/z=199 C₁₁H₇N₂O₂⁺

m/z=158 C₁₀H₈NO⁺

### Example 1-205: 5,5-Dimethyl-1-(2-aminoquinolin-4-ylmethyl)-3-(4-trifluoro-methoxyphenyl)imidazolidine-2,4-dione

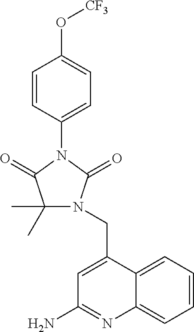 [SMILES: CC1(C)C(=O)N(c2ccc(OC(F)(F)F)cc2)C(=O)N1Cc1cc(N)nc2ccccc12]

0.44 g of tosyl chloride is added to a solution of 0.8 g of 5,5-dimethyl-1-(N-oxyquinolin-4-ylmethyl)-3-(4-trifluoromethoxyphenyl)imidazolidine-2,4-dione in 10 ml of chloroform, under an inert atmosphere of argon at a temperature in the region of 5° C. After stirring for 30 minutes at this same temperature, 1.5 ml of 32% aqueous ammonia are added. The temperature is allowed to rise to 20° C. The reaction medium is stirred at a temperature in the region of 20° C. for 16 hours. 100 ml of water are added. After separation of the phases by settling, the organic phase is dried over sodium sulfate, filtered and then concentrated under reduced pressure. The solid obtained is purified by flash chromatography (SiO₂, EtOAc as eluent, Ar). The fractions containing the product are concentrated under reduced pressure. 200 mg of 5,5-dimethyl-1-(2-aminoquinolin-4-ylmethyl)-3-(4-trifluoromethoxyphenyl)imidazolidine-2,4-dione are thus obtained, the characteristics of which product are as follows:

¹H NMR spectrum (300 MHz, (CD₃)₂SO d6, δ in ppm): 1.49 (s: 6H); 4.96 (broad s: 2H); 6.36 (broad s: 2H); 6.81 (s: 1H); 7.23 (mt: 1H); 7.51 (mt: 2H); 7.58 (broad d, J=9 Hz: 2H); 7.68 (broad d, J=9 Hz: 2H); 7.91 (d, J=8.5 Hz: 1H).

Mass IE m/z=444M⁺. base peak

m/z=429 (M-CH₃)⁺

m/z=198 C₁₁H₈N₃O⁺

m/z=158 C₁₀H₁₀N₂⁺

### Example 1-206: 5,5-Dimethyl-1-(N-oxyquinolin-4-ylmethyl)-3-(4-trifluoro-methoxyphenyl)imidazolidine-2,4-dione

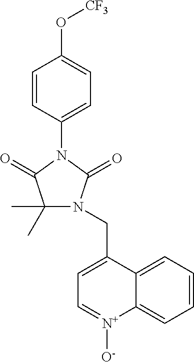 [SMILES: CC1(C)C(=O)N(c2ccc(OC(F)(F)F)cc2)C(=O)N1Cc1cc[n+]([O-])c2ccccc12]

0.78 g of m-chloroperbenzoic acid is added to a solution of 1.95 g of 5,5-dimethyl-1-(quinolin-4-ylmethyl)-3-(4-trifluoromethoxyphenyl)imidazolidine-2,4-dione in 200 ml of chloroform and 10 ml of methanol, under an inert atmosphere of argon, at a temperature in the region of 20° C. The reaction medium is stirred at a temperature in the region of 20° C. for 4 hours. A further 0.8 g of m-chloroperbenzoic acid is added. The reaction medium is concentrated under reduced pressure. The product is crystallized from isopropyl ether to give 1.76 g of 5,5-dimethyl-1-(N-oxyquinolin-4-ylmethyl)-3-(4-trifluoromethoxyphenyl)imidazolidine-2,4-dione, the characteristics of which product are as follows:

¹H NMR spectrum (300 MHz, (CD₃)₂SO d6, δ in ppm): 1.45 (s: 6H); 5.09 (broad s: 2H); 7.55 (broad d, J=8.5 Hz: 2H); 7.63 (d, J=4.5 Hz: 1H); 7.68 (broad d, J=8.5 Hz: 2H); 7.85 (broad t, J=8.5 Hz: 1H); 7.91 (broad t, J=8.5 Hz: 1H); 8.35 (broad d, J=8.5 Hz: 1H); 8.56 (d, J=4.5 Hz: 1H); 8.64 (broad d, J=8.5 Hz: 1H).

Mass ES m/z=446 MH⁺ base peak

### Example 1-207: 5,5-Dimethyl-1-(2-chloroquinolin-4-ylmethyl)-3-(4-trifluoro-methoxyphenyl)imidazolidine-2,4-dione

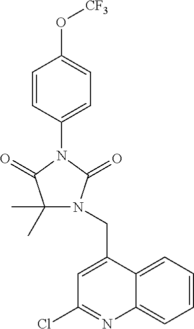 [SMILES: CC1(C)C(=O)N(c2ccc(OC(F)(F)F)cc2)C(=O)N1Cc1cc(Cl)nc2ccccc12]

0.17 ml of phosphorus oxychloride is added to a solution of 4 ml of DMF and 2 ml of toluene, under an inert atmosphere of argon at a temperature in the region of 5° C. After stirring for 30 minutes at this same temperature, 0.4 g of 5,5-dimethyl-1-(N-oxyquinolin-4-ylmethyl)-3-(4-trifluoromethoxyphenyl)imidazolidine-2,4-dione in 10 ml of toluene is added. The reaction medium is stirred at a temperature in the region of 5° C. for 2 and a half hours. The temperature is allowed to return to 20° C. The reaction medium is concentrated under reduced pressure. The residue obtained is taken up in 50 ml of EtOAc and washed with saturated NaHCO₃ solution. After separation of the phases by settling, the organic phase is dried over sodium sulfate, filtered and then concentrated under reduced pressure. The product is crystallized from isopropyl ether to give 0.37 g of 5,5-dimethyl-1-(2-chloroquinolin-4-ylmethyl)-3-(4-trifluoromethoxyphenyl)imidazolidine-2,4-dione, the characteristics of which product are as follows:

¹H NMR spectrum (300 MHz, (CD₃)₂SO d6, δ in ppm): 1.50 (s: 6H); 5.17 (broad s: 2H); 7.56 (broad d, J=8.5 Hz: 2H); 7.71 (broad d, J=8.5 Hz: 2H); 7.76 (s: 1H); 7.76 (split t, J=8 and 1.5 Hz: 1H); 7.89 (split t, J=8 and 1.5 Hz: 1H); 8.03 (broad d, J=8 Hz: 1H); 8.31 (broad d, J=8 Hz: 1H).

Mass IE m/z=463 M⁺. base peak

m/z=448 (M-CH₃)⁺

m/z=428 (M-Cl)⁺

m/z=358 (m/z=428-C₄H₆O)⁺

m/z=217 C₁₁H₆N₂OCl⁺

m/z=176 C₁₀H₇NCl⁺

### Example 1-208: 5,5-Dimethyl-1-(2-chloropyrid-4-ylmethyl)-3-(4-trifluoro-methoxyphenyl)imidazolidine-2,4-dione

0.028 g of sodium hydride is added to a solution of 0.1 g of 5,5-dimethyl-3-(4-trifluoro-methoxyphenyl)imidazolidine-2,4-dione in 2 ml of anhydrous dimethylformamide, under an inert atmosphere of argon at a temperature in the region of 20° C. Stirring is continued at this temperature for 30 minutes. A solution of 0.1 g of 2-chloro-4-(bromomethyl)pyridine in 2 ml of dimethylformamide is added, followed by addition of ice-cold water after reaction for 10 minutes. The reaction medium is placed on a cartridge 20 mm in diameter packed with 17 g of octadecyl-grafted 50 μm silica conditioned successively with acetonitrile and then with water. The elution is performed by gradient using the mixture (water/acetonitrile) of from 0 to 100% acetonitrile. The fractions containing the expected product are concentrated under reduced pressure. 0.130 g of crude product is thus obtained, which product is purified by a double chromatography using a cartridge packed with 10 g of 20-40 μm of conditioned silica, and then eluted with a mixture (cyclohexane/ethyl acetate), (8/2) (v/v) at a flow rate of 5 ml/minute. The fractions between 30 and 75 ml are concentrated under reduced pressure. 0.1 g of 5,5-dimethyl-1-(2-chloropyrid-4-ylmethyl)-3-(4-trifluoromethoxyphenyl)imidazolidine-2,4-dione is thus obtained in the form of a white powder, the characteristics of which product are as follows:

m.p. 134° C.

Mass IE m/z=413 M⁺. base peak

m/z=398 (M-CH₃)⁺

m/z=203 C₈H₄NO₂F₃⁺.

m/z=167 C₇H₄N₂OCl⁺

m/z=126 C₆H₅NCl⁺

¹H NMR spectrum (300 MHz, (CD₃)₂SO d6, δ in ppm): 1.44 (s: 6H); 4.67 (s: 2H); 7.50 (broad d, J=5.5 Hz: 1H); 7.54 (broad d, J=9 Hz: 2H); 7.61 (broad s: 1H); 7.66 (broad d, J=9 Hz: 2H); 8.40 (d, J=5.5 Hz: 1H).

The compound 5,5-dimethyl-3-(4-trifluoromethoxyphenyl)imidazolidine-2,4-dione is described in Example 1-63.

### Example 1-209: 5,5-Dimethyl-1-(2-ethoxypyrid-4-ylmethyl)-3-(4-trifluoro-methoxyphenyl)imidazolidine-2,4-dione

0.015 g of sodium hydride is added to a solution of 0.175 g of 5,5-dimethyl-3-(4-trifluoromethoxyphenyl)imidazolidine-2,4-dione in 2 ml of anhydrous dimethylformamide, under an inert atmosphere of argon at a temperature in the region of 20° C. Stirring is maintained at this temperature for 30 minutes. A solution of 0.185 g of 2-ethoxy-4-(bromomethyl)pyridine in 2 ml of anhydrous dimethylformamide is added, followed by addition of ice-cold water after reaction for 10 minutes. The reaction mixture is placed on a cartridge 37 mm in diameter packed with 65 g of octadecyl-grafted 40-60 μm silica conditioned successively with the mixture (water/acetonitrile) (5/95) (v/v) and then with the mixture (water/acetonitrile) (95/5) (v/v). The elution is performed with a mixture (water/acetonitrile) (95/5) (v/v) over 20 minutes, followed by a linear gradient of from 5% to 95% acetonitrile over 60 minutes, at a flow rate of 10 ml/minute. The fractions between 700 and 760 ml are concentrated under reduced pressure. 0.145 g of 5,5-dimethyl-1-(2-ethoxypyrid-4-ylmethyl)-3-(4-trifluoromethoxyphenyl)imidazolidine-2,4-dione is thus obtained, the characteristics of which product are as follows:

Mass IE m/z=423 M⁺.

m/z=408 (M-CH₃)⁺ base peak

m/z=395 (M-C₂H₄)⁺.

m/z=203 C₈H₄NO₂F₃⁺.

¹H NMR spectrum (300 MHz, (CD₃)₂SO d6, δ in ppm): 1.32 (t, J=7 Hz: 3H); 1.42 (s: 6H); 4.30 (q, J=7 Hz: 2H); 4.58 (broad s: 2H); 6.85 (broad s: 1H); 7.01 (broad d, J=5.5 Hz: 1H); 7.52 (broad d, J=8.5 Hz: 2H); 7.63 (broad d, J=8.5 Hz: 2H); 8.10 (d, J=5.5 Hz: 1H).

The compound 5,5-dimethyl-3-(4-trifluoromethoxyphenyl)imidazolidine-2,4-dione is described in Example 1-63.

### Example 1-210: 5,5-Dimethyl-1-(2-ethylpyrid-4-ylmethyl)-3-(4-trifluoro-methoxyphenyl)imidazolidine-2,4-dione

0.009 g of sodium hydride is added to a solution of 0.064 g of 5,5-dimethyl-3-(4-tri-fluoromethoxyphenyl)imidazolidine-2,4-dione in 1 ml of anhydrous dimethylformamide, under an inert atmosphere of argon, at a temperature in the region of 20° C. Stirring is continued at this temperature for 30 minutes. A solution of 0.062 g of 2-ethyl-4-(bromomethyl)pyridine in 0.5 ml of anhydrous dimethylformamide is added, followed by addition of ice-cold water after reaction for 10 minutes. The reaction mixture is placed on a cartridge 27 mm in diameter packed with 25 g of octadecyl-grafted 40-60 μm silica conditioned successively with the mixture (water/acetonitrile) (5/95) (v/v) and then with the mixture (water/acetonitrile) (95/5) (v/v). The elution is performed with a mixture (water/acetonitrile) (95/5) (v/v) over 20 minutes, followed by a linear gradient of from 5% to 95% acetonitrile over 60 minutes, at a flow rate of 10 ml/minute. The fractions between 750 and 790 ml are concentrated under reduced pressure. 0.06 g of 5,5-dimethyl-1-(2-ethylpyrid-4-ylmethyl)-3-(4-trifluoromethoxyphenyl)imidazolidine-2,4-dione is thus obtained, the characteristics of which product are as follows:

Mass IE m/z=407 M⁺. base peak

m/z=392 (M-CH₃)⁺

m/z=203 C₈H₄NO₂F₃⁺.

m/z=120 C₈H₁₀N⁺

¹H NMR spectrum (400 MHz, (CD₃)₂SO d6, δ in ppm): 1.27 (t, J=7.5 Hz: 3H); 1.44 (s: 6H); 2.75 (q, J=7.5 Hz: 2H); 4.54 (s: 2H); 7.10 (broad d, J=5.5 Hz: 1H); 7.15 (broad s: 1H); 7.39 (broad d, J=8.5 Hz: 2H); 7.49 (d, J=8.5 Hz: 2H); 8.26 (d, J=5.5 Hz: 1H).

The compound 5,5-dimethyl-3-(4-trifluoromethoxy-phenyl)imidazolidine-2,4-dione is described in Example 1-63.

### Example 1-211: 5,5-Dimethyl-1-(2-chloropyrid-4-ylmethyl)-3-(4-trifluoromethane-sulfanylphenyl)imidazolidine-2,4-dione

0.046 g of sodium hydride is added to a solution of 0.175 g of 5,5-dimethyl-3-(4-trifluoromethylsulfanylphenyl)imidazolidine-2,4-dione in 3 ml of anhydrous dimethyl-formamide, under an inert atmosphere of argon, at a temperature in the region of 20° C. Stirring is continued at this temperature for 30 minutes. A solution of 0.166 g of 2-chloro-4-(bromomethyl)pyridine in 2 ml of anhydrous dimethylformamide is added, followed by addition of ice-cold water after reaction for 10 minutes. The reaction mixture is placed on a cartridge 37 mm in diameter packed with 65 g of octadecyl-grafted 40-60 μm silica conditioned successively with the mixture (water/acetonitrile) (5/95) (v/v) and then with the mixture (water/acetonitrile) (95/5) (v/v). The elution is performed with a mixture (water/acetonitrile) (95/5) (v/v) over 20 minutes, followed by a linear gradient of from 5 to 95% acetonitrile over 60 minutes, at a flow rate of 10 ml/minute. The fractions between 740 and 780 ml are concentrated under reduced pressure. 0.03 g of 5,5-dimethyl-1-(2-chloropyrid-4-ylmethyl)-3-(4-trifluoromethanesulfanylphenyl)imidazolidine-2,4-dione is thus obtained in the form of a white powder, the characteristics of which product are as follows:

m.p. 111° C.

Mass IC

m/z=447 MNH₄⁺

m/z=430 MH⁺ base peak

¹H NMR spectrum (300 MHz, (CD₃)₂SO d6, δ in ppm): 1.44 (s: 6H); 4.67 (broad s: 2H); 7.49 (broad d, J=5.5 Hz: 1H); 7.61 (broad s: 1H); 7.70 (broad d, J=8.5 Hz: 2H); 7.88 (broad d, J=8.5 Hz: 2H); 8.38 (d, J=5.5 Hz: 1H).

### Preparation of 5,5-dimethyl-3-(4-trifluoromethylsulfanylphenyl)imidazolidine-2,4-dione

5.12 ml of triethylamine and 2.8 g of methyl α-aminoisobutyrate hydrochloride are added to a solution of 4 g of 4-trifluoromethylsulfanylphenyl isocyanate in 40 ml of toluene, under an inert atmosphere of argon at a temperature in the region of 20° C. The mixture thus obtained is refluxed for 24 hours and then cooled to a temperature in the region of 20° C. The reaction mixture is concentrated to dryness under reduced pressure and the residue obtained is taken up in ethyl ether and filtered. 5.3 g of 5,5-dimethyl-3-(4-trifluoromethylsulfanylphenyl)imidazolidine-2,4-dione are thus obtained, the characteristics of which product are as follows:

Mass IC

m/z=322 MNH₄⁺

m/z=102 triethylamineH⁺ base peak

¹H NMR spectrum (300 MHz, (CD₃)₂SO d6, δ in ppm): 1.44 (s: 6H); 7.62 (broad d, J=8.5 Hz: 2H); 7.85 (broad d, J=8.5 Hz: 2H); 8.72 (unresolved complex: 1H).

The insoluble material thus obtained is taken up in dichloromethane and then washed with water. 2.76 g of 5,5-dimethyl-3-(4-trifluoromethylsulfanylphenyl)imidazolidine-2,4-dione are thus obtained, which product is used for the subsequent synthesis.

### Example 1-212: 5,5-Dimethyl-1-(2-ethoxypyrid-4-yl-methyl)-3-(4-trifluoro-methanesulfanylphenyl)imidazolidine-2,4-dione

0.049 g of sodium hydride is added to a solution of 0.185 g of 5,5-dimethyl-3-(4-trifluoromethylsulfanylphenyl)imidazolidine-2,4-dione in 3.2 ml of anhydrous dimethyl-formamide, under an inert atmosphere of argon at a temperature in the region of 20° C.

Stirring is continued at this temperature for 30 minutes. A solution of 0.184 g of 2-ethoxy-4-(bromomethyl)pyridine in 2 ml of dimethylformamide is added, followed by addition of ice-cold water after reaction for 10 minutes. The reaction mixture is placed on a cartridge 37 mm in diameter packed with 65 g of octadecyl-grafted 40-60 μm silica conditioned successively with the mixture (water/acetonitrile) (5/95) (v/v) and then the mixture (water/acetonitrile) (95/5) (v/v). The elution was performed with a mixture (water/acetonitrile) (95/5) (v/v) over 20 minutes, followed by a linear gradient of from 5% to 95% of acetonitrile over 60 minutes, at a flow rate of 10 ml/minute. The fractions between 520 and 700 ml are concentrated under reduced pressure. The residue thus obtained is purified by flash chromatography using a cartridge packed with 2 g of conditioned 15-35 μm silica, and then eluted with a mixture (cyclohexane/ethyl acetate) (9/1) (v/v). The fractions containing the expected product are concentrated under reduced pressure. 0.01 g of 5,5-dimethyl-1-(2-ethoxypyrid-4-ylmethyl)-3-(4-trifluoromethanesulfanylphenyl)imidazolidine-2,4-dione is thus obtained, the characteristics of which product are as follows:

Mass IE

m/z=439 M⁺.

m/z=424 (M-CH₃)⁺ base peak

m/z=411 (M-C₂H₄)⁺.

m/z=219 C₈H₄NOSF₃⁺.

¹H NMR spectrum (300 MHz, (CD₃)₂SO d6, δ in ppm): 1.32 (t, J=7 Hz: 3H); 1.42 (s: 6H); 4.30 (q, J=7 Hz: 2H); 4.58 (broad s: 2H); 6.86 (broad s: 1H); 7.01 (broad d, J=5.5 Hz: 1H); 7.69 (broad d, J=8.5 Hz: 2H); 7.88 (broad d, J=8.5 Hz: 2H); 8.10 (broad d, J=5.5 Hz: 1H).

The compound 5,5-dimethyl-3-(4-trifluoromethylsulfanylphenyl)imidazolidine-2,4-dione is described in Example 1-210.

### Example 1-213: 5,5-Dimethyl-1-(2-ethylpyrid-4-yl-methyl)-3-(4-trifluoromethane-sulfanylphenyl)imidazolidine-2,4-dione

0.021 g of sodium hydride is added to a solution of 0.135 g of 5,5-dimethyl-3-(4-trifluoromethylsulfanylphenyl)imidazolidine-2,4-dione in 2 ml of anhydrous dimethyl-formamide, under an inert atmosphere of argon at a temperature in the region of 20° C. Stirring is continued at this temperature for 30 minutes. A solution of 0.126 g of 2-ethyl-4-(bromomethyl)pyridine in 1 ml of anhydrous dimethylformamide is added, followed by addition of ice-cold water after reaction for 10 minutes. The reaction mixture is placed on a cartridge 37 mm in diameter packed with 65 g of octadecyl-grafted 40-60 μm silica conditioned successively with the mixture (water/acetonitrile) (5/95) (v/v) and then the mixture (water/acetonitrile) (95/5) (v/v). The elution was performed with a mixture (water/acetonitrile) (95/5) (v/v) over 20 minutes, followed by a linear gradient of from 5% to 95% of acetonitrile over 60 minutes and by an elution with 100% acetonitrile for 10 minutes, at a flow rate of 10 ml/minute. The fractions between 800 and 880 ml are concentrated under reduced pressure. 0.120 g of 5,5-dimethyl-1-(2-ethylpyrid-4-ylmethyl)-3-(4-trifluoromethanesulfanylphenyl)imidazolidine-2,4-dione is thus obtained, the characteristics of which product are as follows:

Mass IE

m/z=423 M⁺. base peak

m/z=408 (M-CH₃)⁺

m/z=219 C₈H₄NOSF₃⁺.

m/z=120 C₈H₁₀N⁺

¹H NMR spectrum (400 MHz, (CD₃)₂SO d6, δ in ppm): 1.23 (t, J=7.5 Hz: 3H); 1.42 (s: 6H); 2.75 (q, J=7.5 Hz: 2H); 4.62 (broad s: 2H); 7.25 (broad d, J=5.5 Hz: 1H); 7.30 (broad s: 1H); 7.70 (d, J=8.5 Hz: 2H); 7.88 (d, J=8.5 Hz: 2H); 8.43 (d, J=5.5 Hz: 1H).

The compound 5,5-dimethyl-3-(4-trifluoromethylsulfanylphenyl)imidazolidine-2,4-dione is described in Example 1-210.

### Example 1-214: 5,5-Dimethyl-1-(2-bromopyrid-4-ylmethyl)-3-(4-trifluoro-methoxyphenyl)imidazolidine-2,4-dione

0.023 g of sodium hydride is added to a solution of 0.081 g of 5,5-dimethyl-3-(4-trifluoromethoxyphenyl)imidazolidine-2,4-dione in 2 ml of anhydrous dimethylformamide, under an inert atmosphere of argon at a temperature in the region of 20° C. Stirring is continued at this temperature for 35 minutes. A solution of 0.071 g of 2-bromo-4-(bromomethyl)pyridine in 1 ml of anhydrous dimethylformamide is added, followed by addition of ice-cold water after reaction for 15 minutes. The reaction mixture is placed on a cartridge 16 mm in diameter packed with 5 g of octadecyl-grafted 40-60 μm silica conditioned successively with the mixture (water/acetonitrile) (5/95) (v/v) and then the mixture (water/acetonitrile) (95/5) (v/v). The elution was performed with a linear gradient of from 5% to 95% of acetonitrile over 30 minutes then with a mixture (water/acetonitrile) (5/95) (v/v) over 10 minutes, at a flow rate of 5 ml/minute. The fractions between 90 and 100 ml are concentrated under reduced pressure. 0.015 g of 5,5-dimethyl-1-(2-bromopyrid-4-ylmethyl)-3-(4-trifluoromethoxyphenyl)imidazolidine-2,4-dione is thus obtained in the form of a white foam, the characteristics of which product are as follows:

Mass IE

m/z=457 M⁺. base peak

m/z=442 (M-CH₃)⁺

m/z=211 C₇H₄N₂OBr⁺

m/z=203 C₈H₄NO₂F₃⁺.

m/z=170 C₆H₅NBr⁺

¹H NMR spectrum (300 MHz, (CD₃)₂SO d6, δ in ppm): 1.44 (s: 6H); 4.67 (broad s: 2H); 7.52 (broad d, J=5.5 Hz: 1H); 7.54 (broad d, J=9 Hz: 2H); 7.66 (broad d, J=9 Hz: 2H); 7.75 (broad s: 1H); 8.37 (d, J=5.5 Hz: 1H).

The compound 5,5-dimethyl-3-(4-trifluoromethoxyphenyl)imidazolidine-2,4-dione is described in Example 1-63.

### Example 1-215: 5,5-Dimethyl-1-(2-fluoropyrid-4-ylmethyl)-3-(4-trifluoromethane-sulfanylphenyl)imidazolidine-2,4-dione

0.025 g of sodium hydride is added to a solution of 0.096 g of 5,5-dimethyl-3-(4-trifluoromethylsulfanylphenyl)imidazolidine-2,4-dione in 2 ml of anhydrous dimethyl-formamide, under an inert atmosphere of argon at a temperature in the region of 20° C. Stirring is continued at this temperature for 25 minutes. A solution of 0.060 g of 2-fluoro-4-(bromomethyl)pyridine in 1 ml of anhydrous dimethylformamide is added, followed by addition of ice-cold water after reaction for 15 minutes. The reaction mixture is placed on a cartridge 16 mm in diameter packed with 5 g of octadecyl-grafted 40-60 μm silica conditioned successively with the mixture (water/acetonitrile) (5/95) (v/v) and then the mixture (water/acetonitrile) (95/5) (v/v). The elution was performed with a linear gradient of from 5% to 95% of acetonitrile over 30 minutes then with a mixture (water/acetonitrile (5/95) (v/v) over 10 minutes, at a flow rate of 5 ml/minute. The fractions between 105 and 125 ml are concentrated under reduced pressure. 0.069 g of 5,5-dimethyl-1-(2-fluoropyrid-4-ylmethyl)-3-(4-trifluoromethanesulfanylphenyl)imidazolidine-2,4-dione is thus obtained in the form of a white solid, the characteristics of which are as follows:

m.p.: 82° C.

Mass IE

m/z=413 M⁺. base peak

m/z=398 (M-CH₃)⁺

m/z=219 C₈H₄NOSF₃⁺.

m/z=151 C₇H₄N₂OF⁺

m/z=110 C₆H₅NF⁺

¹H NMR spectrum (300 MHz, (CD₃)₂SO d6, δ in ppm): 1.45 (s: 6H); 4.72 (broad s: 2H); 7.29 (broad s: 1H); 7.43 (broad d, J=5.5 Hz: 1H); 7.71 (broad d, J=8.5 Hz: 2H); 7.89 (broad d, J=8.5 Hz: 2H); 8.23 (d, J=5.5 Hz: 1H).

The compound 5,5-dimethyl-3-(4-trifluoromethylsulfanylphenyl)imidazolidine-2,4-dione is described in Example 1-210.

### Example 1-216: 5,5-Dimethyl-1-(2-cyanopyrid-4-ylmethyl)-3-(4-trifluoromethane-sulfanylphenyl)imidazolidine-2,4-dione

0.037 g of sodium hydride is added to a solution of 0.139 g of 5,5-dimethyl-3-(4-trifluoromethylsulfanylphenyl)imidazolidine-2,4-dione in 4 ml of anhydrous dimethyl-formamide, under an inert atmosphere of argon at a temperature in the region of 20° C. Stirring is continued at this temperature for 30 minutes. A solution of 0.090 g of 2-cyano-4-(bromomethyl)pyridine in 1 ml of anhydrous dimethylformamide is added, followed by addition of ice-cold water after reaction for 15 minutes. The reaction mixture is placed on a cartridge 20 mm in diameter packed with 10 g of octadecyl-grafted 40-60 μm silica conditioned successively with the mixture (water/acetonitrile) (5/95) (v/v) and then the mixture (water/acetonitrile) (95/5) (v/v). The elution was performed with a linear gradient of from 5% to 95% of acetonitrile over 30 minutes then with a mixture (water-acetonitrile) (5/95) (v/v) over 10 minutes, at a flow rate of 5 ml/minute. The fractions between 210 and 230 ml are concentrated under reduced pressure. 0.1 g of 5,5-dimethyl-1-(2-cyanopyrid-4-ylmethyl)-3-(4-trifluoromethanesulfanylphenyl)imidazolidine-2,4-dione is thus obtained in the form of a solid, the characteristics of which are as follows:

m.p.: 148° C.

Mass IE

m/z=420 M⁺. base peak

m/z=405 (M-CH₃)⁺

m/z=219 C₈H₄NOSF₃⁺.

m/z=158 C₈H₄N₃O⁺

m/z=117 C₇H₅N₂⁺

¹H NMR spectrum (300 MHz, (CD₃)₂SO d6, δ in ppm): 1.44 (s: 6H); 4.74 (broad s: 2H); 7.72 (broad d, J=9 Hz: 2H); 7.81 (dd, J=5.5 and 2 Hz: 1H); 7.89 (broad d, J=9 Hz: 2H); 8.15 (broad s: 1H); 8.72 (broad d, J=5.5 Hz: 1H).

The compound 5,5-dimethyl-3-(4-trifluoromethylsulfanylphenyl)imidazolidine-2,4-dione is described in Example 1-210.

### Example 1-217: 5,5-Dimethyl-1-(2-hydroxycarbonylpyrid-4-ylmethyl)-3-(4-trifluoromethanesulfanylphenyl)imidazolidine-2,4-dione

A solution of 0.09 g of 5,5-dimethyl-1-(2-cyanopyrid-4-ylmethyl)-3-(4-trifluoromethane-sulfanylphenyl)imidazolidine-2,4-dione in 5 ml of 5N hydrochloric acid is refluxed for about 16 hours. The reaction mixture is concentrated under reduced pressure and the crude product thus obtained is purified by flash chromatography using a cartridge 20 mm in diameter packed with 10 g of conditioned 20-40 μm silica, and then eluted with a mixture (dichloromethane/methanol) (9/1) (v/v) at a flow rate of 8 ml/minute. The fractions between 40 and 200 ml are concentrated under reduced pressure. 0.06 g of 5,5-dimethyl-1-(2-hydroxycarbonylpyrid-4-ylmethyl)-3-(4-trifluoromethanesulfanylphenyl)imidazolidine-2,4-dione is thus obtained, the characteristics of which product are as follows:

Mass IE

m/z=439 M⁺.

m/z=395 (M-CONH₂)⁺ base peak

m/z=219 C₈H₄NOSF₃⁺.

m/z=185 C₇H₉N₂O₂S⁺

¹H NMR spectrum (300 MHz, (CD₃)₂SO d6, δ in ppm): 1.45 (s: 6H); 4.76 (broad s: 2H); 7.70 (d, J=8.5 Hz: 2H); 7.72 (mt: 1H); 7.89 (d, J=8.5 Hz: 2H); 8.12 (broad s: 1H); 8.68 (d, J=5.5 Hz: 1H).

### Example 1-218: 5,5-Dimethyl-1-[2-(methylamino)carbonylpyrid-4-ylmethyl]-3-(4-trifluoromethanesulfanylphenyl)imidazolidine-2,4-dione

0.025 g of methylamine hydrochloride, 0.005 g of hydroxybenzotriazole hydrate, 0.105 ml of triethylamine and 72 mg of 1-(3-dimethylaminopropyl)-3-ethylcarbodiimide hydrochloride are successively added to a solution of 0.055 g of 5,5-dimethyl-1-(2-hydroxycarbonylpyrid-4-ylmethyl)-3-(4-trifluoromethanesulfanylphenyl)imidazolidine-2,4-dione in 2 ml of dichloromethane, under an inert atmosphere of argon, at a temperature in the region of 20° C. Stirring is continued at this temperature for about 16 hours. The reaction mixture is washed with water and the organic phase is dried over magnesium sulfate, filtered and then concentrated under reduced pressure. The crude product thus obtained is purified by flash chromatography using a cartridge 16 mm in diameter packed with 5 g of conditioned 20-40 m silica, and then eluted with a mixture (dichloromethane/methanol) (95/05) (v/v) at a flow rate of 10 ml/minute. The fractions containing the expected product are concentrated under reduced pressure. 0.013 g of 5,5-dimethyl-1-[2-(methylamino)carbonylpyrid-4-ylmethyl]-3-(4-trifluoromethanesulfanylphenyl)imidazolidine-2,4-dione is thus obtained in the form of a white powder, the characteristics of which are as follows:

Mass IE

m/z=452 M⁺.

m/z=395 (M-C₂H₃ON)⁺ base peak

m/z=219 C₈H₄NOSF₃⁺.

m/z=148 C₈H₈N₂O⁺

¹H NMR spectrum (300 MHz, (CD₃)₂SO d6, δ in ppm): 1.44 (s: 6H); 2.85 (d, J=5 Hz: 3H); 4.76 (broad s: 2H); 7.67 (dd, J=5 and 2 Hz: 1H); 7.70 (broad d, J=8.5 Hz: 2H); 7.89 (broad d, J=8.5 Hz: 2H); 8.10 (broad s: 1H); 8.60 (d, J=5 Hz: 1H); 8.75 (broad q, J=5 Hz: 1H).

### Example 1-219: 5,5-Dimethyl-1-(2-aminocarbonylpyrid-4-ylmethyl)-3-(4-trifluoro-methanesulfanylphenyl)imidazolidine-2,4-dione

A solution of 0.04 g of 5,5-dimethyl-1-(2-cyanopyrid-4-ylmethyl)-3-(4-trifluoromethane-sulfanylphenyl)imidazolidine-2,4-dione in 1 ml of 98% sulfuric acid is maintained at 40° C. for 30 minutes. The mixture is taken up in 15 ml of ice-cold water and then neutralized with normal sodium hydroxide solution. The solution thus obtained is extracted with 100 ml of ethyl acetate. The organic phase is dried over magnesium sulfate, filtered and then concentrated under reduced pressure. The crude product thus obtained is purified by flash chromatography using a cartridge 16 mm in diameter packed with 5 g of conditioned 20-40 m silica, and then eluted with dichloromethane at a flow rate of 10 ml/minute. The fractions containing the expected product are concentrated under reduced pressure. 0.017 g of 5,5-dimethyl-1-(2-aminocarbonylpyrid-4-ylmethyl)-3-(4-trifluoromethanesulfanylphenyl)imidazolidine-2,4-dione is thus obtained in the form of an amorphous white powder, the characteristics of which product are as follows:

Mass IE

m/z=438 M⁺. base peak

m/z=423 (M-CH₃)⁺

m/z=393 (M-CH₃NO)⁺

m/z=219 C₈H₄NOSF₃⁺.

m/z=135 C₇H₇N₂O⁺

¹H NMR spectrum (300 MHz, (CD₃)₂SO d6, δ in ppm): 1.44 (s: 6H); 4.76 (broad s: 2H); from 7.60 to 7.70 (mt: 2H); 7.71 (broad d, J=8.5 Hz: 2H); 7.89 (broad d, J=8.5 Hz: 2H); 8.11 (broad s: 2H); 8.60 (d, J=5.5 Hz: 1H).

### Example 1-220: 5,5-Dimethyl-1-(2-morpholinopyrid-4-ylmethyl)-3-(4-trifluoro-methoxyphenyl)imidazolidine-2,4-dione

A sealed 2.5 ml tube containing 0.05 g of 5,5-dimethyl-1-(2-chloropyrid-4-ylmethyl)-3-(4-trifluoromethoxyphenyl)imidazolidine-2,4-dione, 0.5 ml of dimethylformamide, 0.034 g of potassium carbonate and 0.021 ml of morpholine is placed in a Personal Chemistry Emrys Optimiser microwave oven. The mixture is irradiated with magnetic stirring at 210° C. for about 50 minutes. The reaction mixture is placed on a Bond Elut Varian cartridge of reference 1225-6053 containing 2 g of SCX phase conditioned with dimethylformamide. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with a mixture (cyclohexane/ethyl acetate) (8/2) (v/v) at a flow rate of 10 ml/minute. The fractions between 160 and 260 ml are concentrated to dryness under reduced pressure. 0.01 g of 5,5-dimethyl-1-(2-morpholinopyrid-4-ylmethyl)-3-(4-trifluoromethoxyphenyl)imidazolidine-2,4-dione is thus obtained in the form of a powder, the characteristics of which product are as follows:

Mass IE

m/z=464 M⁺.

m/z=433 (M-CH₃O)⁺ base peak

m/z=419 (M-C₂H₅O)⁺

m/z=407 (M-C₃H₅O)⁺

m/z=379 (M-C₄H₅NO)⁺

m/z=203 C₈H₄NO₂F₃⁺.

m/z=176 C₁₀H₁₂N₂⁺.

¹H NMR spectrum (300 MHz, (CD₃)₂SO d6, δ in ppm): 1.42 (s: 6H); 3.44 (broad t, J=5 Hz: 4H); 3.72 (broad t, J=5 Hz: 4H); 4.53 (broad s: 2H); 6.75 (broad d, J=5.5 Hz: 1H); 6.85 (broad s: 1H); 7.54 (broad d, J=8.5 Hz: 2H); 7.64 (d, J=8.5 Hz: 2H); 8.10 (d, J=5.5 Hz: 1H).

### Example 1-221: 5,5-Dimethyl-1-(2-morpholinopyrid-4-ylmethyl)-3-(4-trifluoro-methanesulfanylphenyl)imidazolidine-2,4-dione

A sealed 2.5 ml tube containing 0.05 g of 5,5-dimethyl-1-(2-chloropyrid-4-ylmethyl)-3-(4-trifluoromethanesulfanylphenyl)imidazolidine-2,4-dione and 0.5 ml of morpholine is placed in a Personal Chemistry Emrys Optimiser microwave oven. The mixture is irradiated with magnetic stirring at 160° C. for about 70 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6053 containing 2 g of SCX phase conditioned with methanol. The cartridge is washed with methanol and then eluted with 2M ammoniacal methanol. The ammoniacal fractions are concentrated to dryness under reduced pressure. 0.03 g of 5,5-dimethyl-1-(2-morpholinopyrid-4-ylmethyl)-3-(4-trifluoromethanesulfanylphenyl)imidazolidine-2,4-dione is thus obtained in the form of a powder, the characteristics of which are as follows:

Mass IE

m/z=480 M⁺.

m/z=449 (M-CH₃O)⁺ base peak

m/z=435 (M-C₂H₅O)⁺

m/z=423 (M-C₃H₅O)⁺

m/z=219 C₈H₄NOSF₃⁺.

m/z=176 C₁₀H₁₂N₂⁺.

¹H NMR spectrum (300 MHz, (CD₃)₂SO d6, δ in ppm): 1.42 (s: 6H); 3.45 (broad t, J=5 Hz: 4H); 3.71 (broad t, J=5 Hz: 4H); 4.54 (broad s: 2H); 6.75 (broad d, J=5.5 Hz: 1H); 6.84 (broad s: 1H); 7.68 (broad d, J=8.5 Hz: 2H); 7.88 (d, J=8.5 Hz: 2H); 8.09 (d, J=5.5 Hz: 1H).

### Example 1-222: 5,5-Dimethyl-1-(2-dimethylaminopyrid-4-ylmethyl)-3-(4-trifluoro-methanesulfanylphenyl)imidazolidine-2,4-dione

A sealed 2.5 ml tube containing 0.05 g of 5,5-dimethyl-1-(2-chloropyrid-4-ylmethyl)-3-(4-trifluoromethanesulfanylphenyl)imidazolidine-2,4-dione, 0.4 ml of isopropylamine and 0.1 ml of dimethylformamide is placed in a Personal Chemistry Emrys Optimiser microwave oven. The mixture is irradiated with magnetic stirring at 140° C. for about 130 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6053 containing 2 g of SCX phase conditioned with methanol. The cartridge is washed with methanol and then eluted with 2M ammoniacal methanol. The ammoniacal fractions are concentrated to dryness under reduced pressure. 0.013 g of 5,5-dimethyl-1-(2-dimethylaminopyrid-4-ylmethyl)-3-(4-trifluoromethanesulfanylphenyl)imidazolidine-2,4-dione is thus obtained in the form of a powder, the characteristics of which are as follows:

Mass IC

m/z=439 MH⁺ base peak

¹H NMR spectrum (300 MHz, (CD₃)₂SO d6, δ in ppm): 1.42 (s: 6H); 3.03 (s: 6H); 4.53 (broad s: 2H); 6.62 (broad d, J=5.5 Hz: 1H); 6.65 (broad s: 1H); 7.68 (broad d, J=8.5 Hz: 2H); 7.87 (d, J=8.5 Hz: 2H); 8.03 (d, J=5.5 Hz: 1H).

### Example 1-223: 5,5-Dimethyl-1-(2-methylaminopyrid-4-ylmethyl)-3-(4-trifluoro-methanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.5 ml of N-methyl-2-pyrrolidone, 0.016 mg of methylamine hydrochloride and 0.064 ml of triethylamine is placed in a Personal Chemistry Emrys Optimiser microwave oven. The mixture is irradiated with magnetic stirring at 180° C. for about 80 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6054 containing 3 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions containing the expected product are concentrated to dryness under reduced pressure. 0.021 g of 5,5-dimethyl-1-(2-methylaminopyrid-4-ylmethyl)-3-(4-trifluoromethanesulfanylphenyl)imidazolidine-2,4-dione is thus obtained in the form of an amorphous white powder, the characteristics of which product are as follows:

Mass IE

m/z=424 M⁺. base peak

m/z=396 (M-CH₂N)⁺

m/z=219 C₈H₄NOSF₃⁺.

m/z=120 C₇H₈N₂⁺.

¹H NMR spectrum (300 MHz, (CD₃)₂SO d6, δ in ppm): 1.42 (s: 6H); 2.77 (d, J=5 Hz: 3H); 4.48 (broad s: 2H); 6.42 (mt: 1H); 6.46 (broad s: 1H); 6.52 (broad d, J=5.5 Hz: 1H); 7.69 (broad d, J=8.5 Hz: 2H); 7.89 (broad d, J=8.5 Hz: 2H); 7.94 (d, J=5.5 Hz: 1H).

### Example 1-224: 5,5-Dimethyl-1-(2-cyclohexylaminopyrid-4-ylmethyl)-3-(4-trifluoromethanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.2 ml of N-methyl-2-pyrrolidone and 0.4 ml of cyclohexylamine is placed in a Personal Chemistry Emrys Optimiser microwave oven. The mixture is irradiated with magnetic stirring at 200° C. for about 50 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6054 containing 3 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions between 15 and 45 ml are concentrated to dryness under reduced pressure. 0.017 g of 5,5-dimethyl-1-(2-cyclohexylaminopyrid-4-ylmethyl)-3-(4-trifluoromethanesulfanylphenyl)imidazolidine-2,4-dione is thus obtained in the form of an amorphous powder, the characteristics of which product are as follows:

Mass IE

m/z=492 M⁺.

m/z=449 (M-C₃H₇)⁺

m/z=435 (M-C₃H₇)⁺

m/z=410 (M-C₆H₁₀)⁺. base peak

m/z=219 C₈H₄NOSF₃⁺.

m/z=175 C₁₁H₁₅N₂⁺

m/z=98 C₆H₁₂N⁺

¹H NMR spectrum (400 MHz, (CD₃)₂SO d6, δ in ppm): from 1.10 to 1.40 (mt: 5H); 1.43 (s: 6H); from 1.55 to 2.00 (mt: 5H); 3.67 (mt: 1H); 4.45 (s: 2H); 6.28 (d, J=8 Hz: 1H); 6.44 (broad s: 1H); 6.46 (broad d, J=5.5 Hz: 1H); 7.68 (broad d, J=9 Hz: 2H); from 7.85 to 7.95 (mt: 3H).

### Example 1-225: 5,5-Dimethyl-1-(2-isopropylaminopyrid-4-ylmethyl)-3-(4-trifluoro-methanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.2 ml of N-methyl-2-pyrrolidone and 0.4 ml of isopropylamine is placed in a Personal Chemistry Emrys Optimiser microwave oven. The mixture is irradiated with magnetic stirring at 200° C. for about 70 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions between 10 and 35 ml are concentrated to dryness under reduced pressure. 0.008 g of 5,5-dimethyl-1-(2-isopropylaminopyrid-4-ylmethyl)-3-(4-trifluoromethanesulfanylphenyl)imidazolidine-2,4-dione is thus obtained in the form of an amorphous powder, the characteristics of which product are as follows:

Mass IE

m/z=452 M⁺.

m/z=437 (M-CH₃)⁺ base peak

m/z=410 (M-C₃H₆)⁺

m/z=219 C₈H₄NOSF₃⁺.

m/z=134 C₈H₁₀N₂⁺.

m/z=58 C₃H₈N⁺

¹H NMR spectrum (300 MHz, (CD₃)₂SO d6, δ in ppm): 1.13 (d, J=6.5 Hz: 6H); 1.42 (s: 6H); 3.98 (mt: 1H); 4.45 (broad s: 2H); 6.27 (broad d, J=7.5 Hz: 1H); 6.42 (broad s: 1H); 6.46 (broad d, J=5.5 Hz: 1H); 7.68 (broad d, J=8 Hz: 2H); 7.88 (broad d, J=8 Hz: 2H); 7.90 (d, J=5.5 Hz: 1H).

### Example 1-226: 5,5-Dimethyl-1-(2-piperidinopyrid-4-ylmethyl)-3-(4-trifluoro-methanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.2 ml of N-methyl-2-pyrrolidone and 0.4 ml of piperidine is placed in a Personal Chemistry Emrys Optimiser microwave oven. The mixture is irradiated with magnetic stirring at 200° C. for about 30 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 m silica, and then eluted with dichloromethane, at a flow rate of 5 ml/minute. The fractions between 10 and 30 ml are concentrated to dryness under reduced pressure. 0.07 g of 5,5-dimethyl-1-(2-piperidinopyrid-4-ylmethyl)-3-(4-trifluoromethanesulfanylphenyl)imidazolidine-2,4-dione is thus obtained in the form of an amorphous powder, the characteristics of which product are as follows:

Mass IE

m/z=478 M⁺. base peak

m/z=449 (M-C₂H₅)⁺

m/z=422 (M-C₄H₈)⁺.

m/z=395 (M-C₅H₉N)⁺

m/z=219 C₈H₄NOSF₃⁺.

m/z=161 C₁₀H₁₃N₂⁺

m/z=84 C₅H₁₀N⁺

¹H NMR spectrum (300 MHz, (CD₃)₂SO d6, δ in ppm): 1.42 (s: 6H); from 1.45 to 1.65 (mt: 6H); 3.52 (broad t, J=5 Hz: 4H); 4.52 (broad s: 2H); 6.64 (broad d, J=5.5 Hz: 1H); 6.83 (broad s: 1H); 7.68 (d, J=8.5 Hz: 2H); 7.88 (d, J=8.5 Hz: 2H); 8.04 (d, J=5.5 Hz: 1H).

### Example 1-227: 5,5-Dimethyl-1-[2-(4-methylpiperazino)pyrid-4-ylmethyl]-3-(4-trifluoromethanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.2 ml of N-methyl-2-pyrrolidone and 0.4 ml of N-methylpiperazine is placed in a Personal Chemistry Emrys Optimiser microwave oven. The mixture is irradiated with magnetic stirring at 200° C. for about 30 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions between 105 and 135 ml are concentrated to dryness under reduced pressure. 0.028 g of 5,5-dimethyl-1-[2-(4-methylpiperazino)pyrid-4-ylmethyl]-3-(4-trifluoromethanesulfanylphenyl)imidazolidine-2,4-dione is thus obtained in the form of an amorphous powder, the characteristics of which product are as follows:

Mass IE

m/z=493 M⁺.

m/z=423 (M-C₄H₈N)⁺. base peak

m/z=219 C₈H₄NOSF₃⁺.

m/z=176 C₁₀H₁₄N₃⁺

¹H NMR spectrum (300 MHz, (CD₃)₂SO d6, δ in ppm): 1.42 (s: 6H); 2.23 (s: 3H); 2.40 (broad t, J=5 Hz: 4H); 3.50 (broad t, J=5 Hz: 4H); 4.53 (broad s: 2H); 6.70 (broad d, J=5.5 Hz: 1H); 6.85 (broad s: 1H); 7.69 (d, J=8.5 Hz: 2H); 7.88 (d, J=8.5 Hz: 2H); 8.07 (d, J=5.5 Hz: 1H).

### Example 1-228: 5,5-Dimethyl-1-(2-phenylaminopyrid-4-ylmethyl)-3-(4-trifluoro-methanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.2 ml of N-methyl-2-pyrrolidone and 0.4 ml of aniline is placed in a Personal Chemistry Emrys Optimiser microwave oven. The mixture is irradiated with magnetic stirring at 200° C. for about 30 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 m silica, and then eluted with dichloromethane, at a flow rate of 5 ml/minute. The fractions between 40 and 85 ml are concentrated to dryness under reduced pressure. 0.082 g of 5,5-dimethyl-1-(2-phenylaminopyrid-4-ylmethyl)-3-(4-trifluoromethanesulfanylphenyl)imidazolidine-2,4-dione is thus obtained in the form of an amorphous powder, the characteristics of which product are as follows:

Mass IE

m/z=486 M⁺.

m/z=485 (M−H)⁺ base peak

m/z=417 (M-—CF₃)⁺

m/z=182 C₁₂H₁₀N₂⁺.

¹H NMR spectrum (300 MHz, (CD₃)₂SO d6, δ in ppm): 1.46 (s: 6H); 4.55 (broad s: 2H); 6.80 (broad d, J=5.5 Hz: 1H); 6.84 (broad s: 1H); 6.88 (broad t, J=7.5 Hz: 1H); 7.25 (dd, J=8 and 7.5 Hz: 2H); 7.67 (broad d, J=8 Hz: 2H); 7.69 (d, J=8.5 Hz: 2H); 7.88 (broad d, J=8.5 Hz: 2H); 8.01 (d, J=5.5 Hz: 1H); 9.01 (broad s: 1H).

### Example 1-229: 5,5-Dimethyl-1-[2-(4-piperazino)pyrid-4-ylmethyl]-3-(4-trifluoro-methanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.6 ml of N-methyl-2-pyrrolidone and 0.123 g of piperazine is placed in a Personal Chemistry Emrys Optimiser microwave oven. The mixture is irradiated with magnetic stirring at 200° C. for about 30 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions between 45 and 110 ml are concentrated to dryness under reduced pressure. 0.042 g of 5,5-dimethyl-1-[2-(4-piperazino)pyrid-4-ylmethyl]-3-(4-trifluoromethanesulfanylphenyl)imidazolidine-2,4-dione is thus obtained in the form of an amorphous powder, the characteristics of which product are as follows:

Mass IC

m/z=480 MH⁺ base peak

¹H NMR spectrum (300 MHz, (CD₃)₂SO d6, δ in ppm): 1.43 (s: 6H); 2.79 (broad t, J=5 Hz: 4H); 3.41 (broad t, J=5 Hz: 4H); 4.52 (broad s: 2H); 6.69 (broad d, J=5.5 Hz: 1H); 6.82 (broad s: 1H); 7.69 (broad d, J=8.5 Hz: 2H); 7.88 (d, J=8.5 Hz: 2H); 8.06 (d, J=5.5 Hz: 1H).

### Example 1-230: 5,5-Dimethyl-1-(2-ethylaminopyrid-4-ylmethyl)-3-(4-trifluoro-methanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.5 ml of N-methyl-2-pyrrolidone, 0.019 g of ethylamine hydrochloride and 0.064 ml of triethylamine is placed in a Personal Chemistry Emrys Optimiser microwave oven. The mixture is irradiated with magnetic stirring at 200° C. for about 120 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions between 280 and 300 ml are concentrated to dryness under reduced pressure. 0.033 g of 5,5-dimethyl-1-(2-ethyl aminopyrid-4-ylmethyl)-3-(4-trifluoromethanesulfanylphenyl)imidazolidine-2,4-dione is thus obtained in the form of an amorphous powder, the characteristics of which product are as follows:

m.p.=136° C.

Mass IE

m/z=438 M⁺.

m/z=423 (M-CH₃)⁺ base peak

m/z=395 (M-C₂H₅N)⁺

m/z=369 (M-—CF₃)⁺

m/z=121 C₇H₉N₂⁺.

m/z=44 C₂H₆N⁺

¹H NMR spectrum (300 MHz, (CD₃)₂SO d6, δ in ppm): 1.14 (t, J=7 Hz: 3H); 1.44 (s: 6H); 3.26 (mt: 2H); 4.47 (broad s: 2H); 6.40 (broad t, J=5.5 Hz: 1H); 6.45 (broad s: 1H); 6.50 (broad d, J=5.5 Hz: 1H); 7.69 (broad d, J=8.5 Hz: 2H); 7.89 (broad d, J=8.5 Hz: 2H); 7.92 (d, J=5.5 Hz: 1H).

### Example 1-231: 5,5-Dimethyl-1-(2-benzylaminopyrid-4-ylmethyl)-3-(4-trifluoro-methanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.2 ml of N-methyl-2-pyrrolidone and 0.4 ml of benzylamine is placed in a Personal Chemistry Emrys Optimiser microwave oven. The mixture is irradiated with magnetic stirring at 200° C. for about 30 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions between 10 and 50 ml are concentrated to dryness under reduced pressure. 0.035 g of 5,5-dimethyl-1-(2-benzylaminopyrid-4-ylmethyl)-3-(4-trifluoromethanesulfanylphenyl)imidazolidine-2,4-dione is thus obtained in the form of an amorphous powder, the characteristics of which are as follows:

m.p. 144° C.

Mass IE

m/z=500 M⁺. base peak

m/z=431 (M-—CF₃)⁺

m/z=196 C₁₃H₁₂N₂⁺.

m/z=106 C₇H₈N⁺

m/z=91 C₇H₇⁺

¹H NMR spectrum (300 MHz, (CD₃)₂SO d6, δ in ppm): 1.39 (s: 6H); 4.47 (broad s: 2H); 4.48 (d, J=6 Hz: 2H); 6.51 (broad s: 1H); 6.54 (broad d, J=5.5 Hz: 1H); 7.02 (broad t, J=6 Hz: 1H); from 7.15 to 7.35 (mt: 5H); 7.68 (broad d, J=8.5 Hz: 2H); 7.89 (d, J=8.5 Hz: 2H); 7.92 (d, J=5.5 Hz: 1H).

### Example 1-232: 5,5-Dimethyl-1-[2-(4-methoxybenzylamino)pyrid-4-ylmethyl]-3-(4-trifluoromethanesulfanylphenyl)imidazolidine-2,4-dione

A sealed 2.5 ml tube containing 0.1 g of 5,5-dimethyl-1-(2-chloropyrid-4-ylmethyl)-3-(4-trifluoromethanesulfanylphenyl)imidazolidine-2,4-dione, 0.2 ml of N-methyl-2-pyrrolidone and 0.4 ml of para-methoxybenzylamine is placed in a Personal Chemistry Emrys Optimiser microwave oven. The mixture is irradiated with magnetic stirring at 200° C. for about 30 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 16 mm in diameter packed with 2 g of conditioned 15-35 μm silica, and then eluted with dichloromethane, at a flow rate of 5 ml/minute. The fractions between 5 and 55 ml are concentrated to dryness under reduced pressure. 0.035 g of 5,5-dimethyl-1-[2-(4-methoxybenzylamino)pyrid-4-ylmethyl]-3-(4-trifluoromethanesulfanylphenyl)imidazolidine-2,4-dione is thus obtained in the form of an amorphous powder, the characteristics of which product are as follows:

Mass IE

m/z=530 M⁺.

m/z=461 (M-—CF₃)⁺

m/z=136 C₈H₁₀NO⁺

m/z=121 C₈H₉O⁺ base peak

¹H NMR spectrum (300 MHz, (CD₃)₂SO d6, δ in ppm): 1.39 (s: 6H); 3.72 (s: 3H); 4.39 (d, J=5.5 Hz: 2H); 4.46 (broad s: 2H); 6.48 (broad s: 1H); 6.53 (broad d, J=5.5 Hz: 1H); 6.87 (broad d, J=8.5 Hz: 2H); 6.93 (t, J=5.5 Hz: 1H); 7.25 (broad d, J=8.5 Hz: 2H); 7.68 (broad d, J=8.5 Hz: 2H); 7.89 (broad d, J=8.5 Hz: 2H); 7.92 (d, J=5.5 Hz: 1H).

### Example 1-233: 5,5-Dimethyl-1-(2-aminopyrid-4-ylmethyl)-3-(4-trifluoromethane-sulfanylphenyl)imidazolidine-2,4-dione

0.5 ml of trifluoroacetic acid is added to a solution of 0.035 g of 5,5-dimethyl-1-[2-(4-methoxybenzylamino)pyrid-4-ylmethyl]-3-(4-trifluoromethanesulfanylphenyl)imidazolidine-2,4-dione in 0.5 ml of dichloromethane, at a temperature in the region of 20° C. Stirring is continued at this temperature for 5 hours. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. 0.015 g of 5,5-dimethyl-1-(2-aminopyrid-4-ylmethyl)-3-(4-trifluoromethanesulfanylphenyl)imidazolidine-2,4-dione is thus obtained in the form of a powder, the characteristics of which product are as follows:

m.p.: 161° C.

Mass IE

m/z=410 M⁺. base peak

m/z=395 (M-CH₃)⁺

m/z=219 C₈H₄NOSF₃⁺.

m/z=148 C₇H₆N₃O⁺

m/z=107 C₆H₇N₂⁺

¹H NMR spectrum (300 MHz, (CD₃)₂SO d6, δ in ppm): 1.43 (s: 6H); 4.46 (broad s: 2H); 5.86 (broad s: 2H); 6.46 (broad s: 1H); 6.53 (broad d, J=5.5 Hz: 1H); 7.69 (broad d, J=8.5 Hz: 2H); 7.84 (d, J=5.5 Hz: 1H); 7.89 (broad d, J=8.5 Hz: 2H).

### Example 1-234: 5,5-Dimethyl-1-(2-acetamidopyrid-4-ylmethyl)-3-(4-trifluoro-methanesulfanylphenyl)imidazolidine-2,4-dione

A solution of 0.050 g of 5,5-dimethyl-1-(2-aminopyrid-4-ylmethyl)-3-(4-trifluoro-methanesulfanylphenyl)imidazolidine-2,4-dione in 5 ml of acetic anhydride is stirred under an inert atmosphere of argon for 24 hours at a temperature in the region of 80° C. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. 0.006 g of 5,5-dimethyl-1-(2-acetamidopyrid-4-ylmethyl)-3-(4-trifluoromethanesulfanylphenyl)imidazolidine-2,4-dione is thus obtained in the form of a powder, the characteristics of which product are as follows:

Mass IE

m/z=452 M⁺.

m/z=410 (M-C₂H₂O)⁺. base peak

m/z=395 (m/z=410-CH₃)⁺

m/z=219 C₈H₄NOSF₃⁺.

m/z=150 C₈H₁₀N₂O⁺.

m/z=107 C₆H₇N₂⁺

m/z=43 C₂H₃O⁺

¹H NMR spectrum (300 MHz, (CD₃)₂SO d6, δ in ppm): 1.44 (broad s: 6H); 2.11 (s: 3H); 4.65 (broad s: 2H); 7.15 (broad d, J=5.5 Hz: 1H); 7.68 (broad d, J=8.5 Hz: 2H); 7.89 (broad d, J=8.5 Hz: 2H); 8.14 (broad s: 1H); 8.27 (d, J=5.5 Hz: 1H); 10.49 (unresolved complex: 1H).

### Example 1-235: 5,5-Dimethyl-1-(2-tert-butoxycarbonylaminopyrid-4-ylmethyl)-3-(4-trifluoromethanesulfanylphenyl)imidazolidine-2,4-dione

0.05 g of 5,5-dimethyl-1-(2-aminopyrid-4-ylmethyl)-3-(4-trifluoromethanesulfanyl-phenyl)imidazolidine-2,4-dione is added portionwise to a solution of 0.029 g of di-tert-butyl dicarbonate in 0.65 ml of tert-butyl alcohol, under an inert atmosphere of argon at a temperature in the region of 20° C. Stirring is continued at this temperature for 24 hours. The reaction mixture is concentrated to dryness under reduced pressure. The residue thus obtained is purified by flash chromatography using a cartridge 16 mm in diameter packed with 5 g of conditioned 20-40 μm silica, and then eluted with a mixture (cyclohexane/ethyl acetate) (8/2) (v/v). The fractions between 260 and 400 ml are concentrated under reduced pressure. 0.035 g of 5,5-dimethyl-1-(2-tert-butoxycarbonylaminopyrid-4-ylmethyl)-3-(4-trifluoromethanesulfanylphenyl)imidazolidine-2,4-dione is thus obtained, the characteristics of which product are as follows:

Mass IC

m/z=511 MH⁺ base peak

¹H NMR spectrum (300 MHz, (CD₃)₂SO d6, δ in ppm): 1.43 (s: 6H); 1.49 (s: 9H); 4.63 (broad s: 2H); 7.08 (broad dd, J=5.5 and 1.5 Hz: 1H); 7.68 (broad d, J=9 Hz: 2H); 7.84 (broad s: 1H); 7.87 (broad d, J=9 Hz: 2H); 8.19 (d, J=5.5 Hz: 1H); 9.73 (broad s: 1H).

### Example 1-236: 5,5-Dimethyl-1-(2-methylsulfonylaminopyrid-4-ylmethyl)-3-(4-trifluoromethanesulfanylphenyl)imidazolidine-2,4-dione

0.010 ml of methanesulfonyl chloride is added to a solution of 0.050 g of 5,5-dimethyl-1-(2-aminopyrid-4-ylmethyl)-3-(4-trifluoromethanesulfanylphenyl)imidazolidine-2,4-dione in 0.4 ml of pyridine, under an inert atmosphere of argon at a temperature in the region of 20° C.

Stirring is continued at this temperature for 2 hours. The reaction mixture is poured into 50 ml of saturated sodium bicarbonate solution and then extracted with 2×50 ml of ethyl acetate. The organic phase is dried over magnesium sulfate, filtered and then concentrated to dryness under reduced pressure. 0.020 g of 5,5-dimethyl-1-(2-methylsulfonylaminopyrid-4-ylmethyl)-3-(4-trifluoromethanesulfanylphenyl)imidazolidine-2,4-dione is thus obtained in the form of a white powder, the characteristics of which product are as follows: m.p.: 208° C.

Mass IE

m/z=488 M⁺. base peak

m/z=473 (M-CH₃)⁺

m/z=409 (M-SO₂CH₃)⁺

m/z=219 C₈H₄NOSF₃⁺.

m/z=185 C₇H₉N₂O₂S⁺

¹H NMR spectrum (300 MHz, (CD₃)₂SO d6, δ in ppm): 1.45 (s: 6H); 3.27 (unresolved complex: 3H); 4.63 (broad s: 2H); from 6.95 to 7.10 (unresolved complex: 2H); 7.68 (broad d, J=8.5 Hz: 2H); 7.89 (broad d, J=8.5 Hz: 2H); 8.17 (unresolved complex: 1H).

### Example 1-237: 5,5-Dimethyl-1-(2-methoxycarbonylaminopyrid-4-ylmethyl)-3-(4-trifluoromethanesulfanylphenyl)imidazolidine-2,4-dione

0.056 ml of methyl chloroformate is added to a solution of 0.100 g of 5,5-dimethyl-1-(2-aminopyrid-4-ylmethyl)-3-(4-trifluoromethanesulfanylphenyl)imidazolidine-2,4-dione in 1.5 ml of pyridine, under an inert atmosphere of argon at a temperature in the region of 20° C. Stirring is continued at this temperature for 20 hours. A further 0.56 ml of methyl chloroformate is added. Stirring is continued at this temperature for 20 hours. The reaction mixture is taken up in 5 ml of ethyl acetate, and then washed successively with 5 ml of water, 5 ml of saturated sodium chloride solution and then 5 ml of water. The organic phase is dried over magnesium sulfate, filtered and then concentrated to dryness under reduced pressure. The residue obtained is purified on a Kromasil C18, 5 μm column (100×20 mm), eluting with a linear gradient of from 5% to 95% acetonitrile containing 0.07% (v/v) of trifluoroacetic acid (TFA) in water containing 0.07% (v/v) TFA, at a flow rate of 10 ml/minute. The fractions containing the expected product are concentrated to dryness under reduced pressure. The residue obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. 0.024 g of 5,5-dimethyl-1-(2-methoxycarbonylaminopyrid-4-ylmethyl)-3-(4-trifluoromethanesulfanylphenyl)imidazolidine-2,4-dione is thus obtained in the form of a white solid, the characteristics of which product are as follows:

Mass IE

m/z=468 M⁺. base peak

m/z=453 (M-CH₃)⁺

m/z=410 (M-C₂H₂O₂)⁺.

m/z=219 C₈H₄NOSF₃⁺.

m/z=166 C₈H₁₀N₂O₂⁺.

¹H NMR spectrum (300 MHz, (CD₃)₂SO d6, δ in ppm): 1.44 (s: 6H); 3.70 (s: 3H); 4.65 (broad s: 2H); 7.12 (dd, J=5.5 and 2 Hz: 1H); 7.69 (broad d, J=9 Hz: 2H); 7.89 (broad d, J=9 Hz: 2H); 7.90 (broad s: 1H); 8.23 (d, J=5.5 Hz: 1H); 10.18 (broad s: 1H).

### Example 1-238: 5,5-Dimethyl-1-(2-chloropyrid-4-ylmethyl)-3-(3-chloro-4-trifluoro-methanesulfanylphenyl)imidazolidine-2,4-dione

0.1 g of sodium hydride is added to a solution of 0.417 g of 5,5-dimethyl-3-(3-chloro-4-trifluoromethylsulfanylphenyl)imidazolidine-2,4-dione in 20 ml of anhydrous dimethylformamide, under an inert atmosphere of argon at a temperature in the region of 0° C. Stirring is continued at this temperature for 30 minutes. A solution of 0.380 g of 2-chloro-4-(bromomethyl)pyridine in 20 ml of anhydrous dimethylformamide is added, followed by addition of ice-cold water after reaction for 10 minutes. The reaction mixture is taken up in 400 ml of ethyl acetate and then washed with 400 ml of water. The organic phase is dried over magnesium sulfate, filtered and then concentrated to dryness under reduced pressure. The residue thus obtained is purified by flash chromatography using a cartridge 27 mm in diameter packed with 25 g of conditioned 20-40 μm silica, and then eluted with a mixture (cyclohexane/ethyl acetate) (8/2) (v/v). The fractions between 150 and 350 ml are concentrated to dryness under reduced pressure. 0.3 g of 5,5-dimethyl-1-(2-chloropyrid-4-ylmethyl)-3-(3-chloro-4-trifluoromethanesulfanylphenyl)imidazolidine-2,4-dione is thus obtained, the characteristics of which product are as follows:

Mass IE

m/z=463 M⁺. base peak

m/z=448 (M-CH₃)⁺

m/z=253 C₈H₃NOSClF₃⁺.

m/z=167 C₇H₄N₂OCl⁺

m/z=126 C₆H₅NCl⁺

¹H NMR spectrum (300 MHz, (CD₃)₂SO d6, δ in ppm): 1.43 (s: 6H); 4.68 (broad s: 2H); 7.49 (broad d, J=5.5 Hz: 1H); 7.63 (broad s: 1H); 7.71 (dd, J=9 and 3 Hz: 1H); 7.97 (d, J=3 Hz: 1H); 8.05 (d, J=9 Hz: 1H); 8.38 (d, J=5.5 Hz: 1H).

### Preparation of 5,5-dimethyl-3-(3-chloro-4-trifluoromethylsulfanylphenyl)imidazolidine-2,4-dione

A solution of 2 g of 3-chloro-4-trifluoromethylsulfanylaniline in 22 ml of toluene is added over 30 minutes to a suspension of 1.97 g of diphosgene and 0.03 g of vegetable charcoal in 22 ml of toluene, at a temperature in the region of −20° C. The mixture is stirred to a temperature in the region of 20° C. and is then refluxed for 3 hours. The mixture is cooled to a temperature in the region of 20° C. and then filtered through Celite. 1.35 g of methyl α-aminoisobutyrate hydrochloride, 15 ml of toluene and 2.23 ml of triethylamine are added to the filtrate. The mixture thus obtained is refluxed for 24 hours and then cooled to a temperature in the region of 20° C. The reaction mixture is concentrated under reduced pressure. The residue obtained is purified by flash chromatography on a cartridge 37 mm in diameter packed with conditioned 20-40 μm silica and then eluted with a mixture (cyclohexane/ethyl acetate) (5/5) (v/v). The fractions between 110 and 250 ml are concentrated under reduced pressure, and 2 g of 5,5-dimethyl-3-(3-chloro-4-trifluoro-methylsulfanylphenyl)imidazolidine-2,4-dione are thus obtained, the characteristics of which product are as follows:

Mass IE

m/z=338 M⁺. base peak

m/z=253 C₈H₃NOSClF₃⁺.

m/z=184 (m/z=253-—CF₃)⁺

m/z=84 C₄H₆NO⁺

¹H NMR spectrum (300 MHz, (CD₃)₂SO d6, δ in ppm): 1.42 (s: 6H); 7.63 (dd, J=8.5 and 2.5 Hz: 1H); 7.90 (d, J=2.5 Hz: 1H); 8.02 (d, J=8.5 Hz: 1H); 8.75 (unresolved complex: 1H).

### Example 1-239: 5,5-Dimethyl-1-[2-(4-methoxybenzylamino)pyrid-4-ylmethyl]-3-(3-chloro-4-trifluoromethanesulfanylphenyl)imidazolidine-2,4-dione

A sealed 2.5 ml tube containing 0.3 g of 5,5-dimethyl-1-(2-chloropyrid-4-ylmethyl)-3-(3-chloro-4-trifluoromethanesulfanylphenyl)imidazolidine-2,4-dione, 0.2 ml of N-methyl-2-pyrrolidone and 0.4 ml of para-methoxybenzylamine is placed in a Personal Chemistry Emrys Optimiser microwave oven. The mixture is irradiated with magnetic stirring at 200° C. for about 70 minutes. The reaction mixture is purified by flash chromatography using a cartridge 37 mm in diameter packed with 50 g of 20-40 μm conditioned silica, and then eluted with a mixture (cyclohexane/ethyl acetate) (8/2) (v/v). The fractions between 470 and 640 ml are concentrated to dryness under reduced pressure. 0.091 g of 5,5-dimethyl-1-[2-(4-methoxybenzylamino)pyrid-4-ylmethyl]-3-(3-chloro-4-trifluoro-methanesulfanylphenyl)imidazolidine-2,4-dione is thus obtained, the characteristics of which are as follows:

Mass IE

m/z=564 M⁺.

m/z=495 (M-—CF₃)⁺

m/z=136 C₈H₁₀NO⁺

m/z=121 C₈H₉O⁺ base peak

¹H NMR spectrum (300 MHz, (CD₃)₂SO d6, δ in ppm): 1.38 (s: 6H); 3.71 (s: 3H); 4.39 (d, J=5.5 Hz: 2H); 4.45 (broad s: 2H); 6.48 (broad s: 1H); 6.53 (broad d, J=5.5 Hz: 1H); 6.85 (d, J=8.5 Hz: 2H); 6.89 (t, J=5.5 Hz: 1H); 7.24 (d, J=8.5 Hz: 2H); 7.69 (dd, J=8.5 and 2.5 Hz: 1H); 7.92 (d, J=5.5 Hz: 1H); 7.95 (d, J=2.5 Hz: 1H); 8.06 (d, J=8.5 Hz: 1H).

### Example 1-240: 5,5-Dimethyl-1-(2-ethylaminopyrid-4-ylmethyl)-3-(3-chloro-4-trifluoromethanesulfanylphenyl)imidazolidine-2,4-dione

A sealed 2.5 ml tube containing 0.1 g of 5,5-dimethyl-1-(2-chloropyrid-4-ylmethyl)-3-(3-chloro-4-trifluoromethanesulfanylphenyl)imidazolidine-2,4-dione, 0.4 ml of N-methyl-2-pyrrolidone, 0.044 g of ethylamine hydrochloride and 0.120 ml of triethylamine is placed in a Personal Chemistry Emrys Optimiser microwave oven. The mixture is irradiated with magnetic stirring at 200° C. for about 70 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. 0.029 g of 5,5-dimethyl-1-(2-ethylaminopyrid-4-ylmethyl)-3-(3-chloro-4-trifluoromethanesulfanylphenyl)imidazolidine-2,4-dione is thus obtained, the characteristics of which are as follows:

Mass IE

m/z=472 M⁺.

m/z=457 (M-CH₃)⁺ base peak

m/z=429 (M-C₂H₅N)⁺

m/z=403 (M-—CF₃)⁺

m/z=121 C₇H₉N₂⁺.

m/z=44 C₂H₆N⁺

¹H NMR spectrum (300 MHz, (CD₃)₂SO d6, δ in ppm): 1.14 (t, J=7 Hz: 3H); 1.43 (s: 6H); 3.27 (mt: 2H); 4.46 (broad s: 2H); 6.38 (broad t, J=5.5 Hz: 1H); 6.45 (broad s: 1H); 6.51 (broad d, J=5.5 Hz: 1H); 7.70 (dd, J=8.5 and 2.5 Hz: 1H); 7.92 (d, J=5.5 Hz: 1H); 7.96 (d, J=2.5 Hz: 1H); 8.06 (d, J=8.5 Hz: 1H).

### Example 1-241: 5,5-Dimethyl-1-(2-methylaminopyrid-4-ylmethyl)-3-(3-chloro-4-trifluoromethanesulfanylphenyl)imidazolidine-2,4-dione

A sealed 2.5 ml tube containing 0.1 g of 5,5-dimethyl-1-(2-chloropyrid-4-ylmethyl)-3-(3-chloro-4-trifluoromethanesulfanylphenyl)imidazolidine-2,4-dione, 0.5 ml of N-methyl-2-pyrrolidone, 0.030 g of methylamine hydrochloride and 0.120 ml of triethylamine is placed in a Personal Chemistry Emrys Optimiser microwave oven. The mixture is irradiated with magnetic stirring at 200° C. for about 70 minutes. The reaction mixture is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. The residue thus obtained is purified by flash chromatography using a cartridge 20 mm in diameter packed with 10 g of 20-40 μm conditioned silica, and then eluted with dichloromethane at a flow rate of 8 ml/minute. The fractions between 70 and 100 ml are concentrated to dryness under reduced pressure. 0.015 g of 5,5-dimethyl-1-(2-methylaminopyrid-4-ylmethyl)-3-(3-chloro-4-trifluoromethanesulfanylphenyl)imidazolidine-2,4-dione is thus obtained in the form of an amorphous white powder, the characteristics of which are as follows:

¹H NMR spectrum (300 MHz, (CD₃)₂SO d6, δ in ppm): 1.43 (s: 6H); 2.77 (d, J=5 Hz: 3H); 4.48 (broad s: 2H); 6.40 (broad q, J=5 Hz: 1H); 6.46 (broad s: 1H); 6.54 (broad d, J=5.5 Hz: 1H); 7.70 (dd, J=8.5 and 3 Hz: 1H); 7.94 (d, J=5.5 Hz: 1H); 7.97 (d, J=3 Hz: 1H); 8.06 (d, J=8.5 Hz: 1H).

### Example 1-242: 5,5-Dimethyl-1-(2-aminopyrid-4-ylmethyl)-3-(3-chloro-4-trifluoro-methanesulfanylphenyl)imidazolidine-2,4-dione

3 ml of trifluoroacetic acid are added to a solution of 0.080 g of 5,5-dimethyl-1-[2-(4-methoxybenzylamino)pyrid-4-ylmethyl]-3-(3-chloro-4-trifluoromethanesulfanylphenyl)-imidazolidine-2,4-dione in 1 ml of dichloromethane, at a temperature in the region of 20° C. Stirring is continued at this temperature for about 16 hours. The reaction mixture is concentrated to dryness under reduced pressure. The residue obtained is purified by preparative LC/MS. The fractions containing the expected product are concentrated to dryness under reduced pressure. The residue thus obtained is taken up in methanol and then placed on a Bond Elut Varian cartridge of reference 1225-6027 containing 5 g of SCX phase conditioned with methanol. The cartridge is washed with methanol and then eluted with 2M ammoniacal methanol. The ammoniacal fractions are concentrated to dryness under reduced pressure. 0.027 g of 5,5-dimethyl-1-(2-aminopyrid-4-ylmethyl)-3-(3-chloro-4-trifluoromethanesulfanylphenyl)imidazolidine-2,4-dione is thus obtained in the form of a powder, the characteristics of which are as follows:

Mass IE

m/z=444 M⁺. base peak

m/z=429 (M-CH₃)⁺

m/z=148 C₇H₆N₃O⁺

m/z=107 C₆H₇N₂⁺

¹H NMR spectrum (300 MHz, (CD₃)₂SO d6, δ in ppm): 1.43 (s: 6H); 4.46 (broad s: 2H); 5.86 (broad s: 2H); 6.46 (broad s: 1H); 6.54 (broad dd, J=5.5 and 1.5 Hz: 1H); 7.70 (dd, J=8.5 and 2.5 Hz: 1H); 7.86 (d, J=5.5 Hz: 1H); 7.97 (d, J=2.5 Hz: 1H); 8.06 (d, J=8.5 Hz: 1H).

### Example 1-243: 5,5-Dimethyl-1-(2,6-dibromopyrid-4-ylmethyl)-3-(4-trifluoro-methoxyphenyl)imidazolidine-2,4-dione

0.036 g of sodium hydride is added to a solution of 0.230 g of 5,5-dimethyl-3-(4-trifluoromethylsulfanylphenyl)imidazolidine-2,4-dione in 2 ml of anhydrous dimethyl-formamide, under an inert atmosphere of argon at a temperature in the region of 20° C. Stirring is continued at this temperature for 30 minutes. A solution of 0.349 g of 2,6-dibromo-4-(bromomethyl)pyridine in 3 ml of anhydrous dimethylformamide is added, followed by addition of ice-cold water after reaction for 20 minutes. The reaction mixture is taken up in ethyl acetate and then washed with water. The organic phase is dried over magnesium sulfate, filtered and then concentrated to dryness under reduced pressure. The residue thus obtained is purified by flash chromatography using a cartridge 27 mm in diameter packed with 25 g of 20-40 μm conditioned silica, and then eluted with dichloromethane. The fractions between 65 and 135 ml are concentrated under reduced pressure. 0.25 g of 5,5-dimethyl-1-(2,6-dibromopyrid-4-ylmethyl)-3-(4-trifluoromethoxy-phenyl)imidazolidine-2,4-dione is thus obtained, the characteristics of which product are as follows:

Mass IC

m/z=569 MNH₄⁺ base peak

m/z=552 MH⁺

m/z=474 (M-Br+2H)⁺

m/z=396 (m/z=474-Br+H)⁺

¹H NMR spectrum (300 MHz, (CD₃)₂SO d6, δ in ppm): 1.45 (s: 6H); 4.67 (broad s: 2H); 7.72 (broad d, J=8 Hz: 2H); 7.83 (s: 2H); 7.89 (broad d, J=8 Hz: 2H).

The compound 5,5-dimethyl-3-(4-trifluoromethylsulfanylphenyl)imidazolidine-2,4-dione is described in Example 1-210.

### Synthesis of the Reagents

### Reference Example 1-208 a: 2-Chloro-4-(bromomethyl)pyridine

A solution of 0.2 g of 2-chloro-4-(hydroxymethyl)pyridine in 2.5 ml of dichloromethane is added dropwise to a solution of 0.706 g of dibromotriphenylphosphorane in 9.5 ml of dichloromethane, under an inert atmosphere of argon at a temperature in the region of 20° C. Stirring is continued at this temperature for about 1 hour. The reaction medium is taken up in 50 ml of dichloromethane and then washed with 3×50 ml of water. The organic phase is dried over magnesium sulfate, filtered and then concentrated to dryness under reduced pressure. The residue thus obtained is purified by flash chromatography using a cartridge 20 mm in diameter packed with 10 g of 20-40 μm conditioned silica, and then eluted with a mixture (cyclohexane/ethyl acetate) (8/2) (v/v) at a flow rate of 8 ml/minute. The fractions between 8 and 24 ml are concentrated under reduced pressure. 0.1 g of 2-chloro-4-(bromomethyl)pyridine is thus obtained.

### Reference Example 1-208 b: 2-chloro-4-(hydroxymethyl)pyridine

2.04 g of sodium borohydride are added portionwise to a solution of 1.7 g of ethyl 2-chloropyridine-4-carboxylate in 20 ml of ethanol, under an inert atmosphere of argon at a temperature in the region of 0° C. The reaction mixture is refluxed with stirring for 3 hours and then concentrated to dryness under reduced pressure. The residue thus obtained is taken up in dichloromethane and then washed with water. The organic phase is dried over magnesium sulfate, filtered and then concentrated to dryness under reduced pressure. 1 g of 2-chloro-4-(hydroxymethyl)pyridine is thus obtained.

### Reference Example 1-208 c: Ethyl 2-chloropyridine-4-carboxylate

1 ml of concentrated sulfuric acid is added to a solution of 2.2 g of 2-chloropyridine-4-carboxylic acid in 30 ml of ethanol. The reaction mixture is refluxed with stirring for 16 hours and then concentrated to dryness under reduced pressure. The residue thus obtained is taken up in 30 ml of water and then extracted with 3×30 ml of dichloromethane. The organic phase is dried over magnesium sulfate, filtered and then concentrated to dryness under reduced pressure. The oil thus obtained is purified by filtration through 13 g of silica, eluting with dichloromethane. The fractions containing the expected product are concentrated to dryness under reduced pressure. 2.1 g of ethyl 2-chloropyridine-4-carboxylate are thus obtained.

### Reference Example 1-209 a: 2-ethoxy-4-(bromomethyl)pyridine

A solution of 0.31 g of 2-ethoxy-4-(hydroxymethyl)pyridine in 6 ml of dichloromethane is added dropwise to a solution of 1.025 g of dibromotriphenylphosphorane in 12 ml of dichloromethane, under an inert atmosphere of argon at a temperature in the region of 20° C. Stirring is continued at this temperature for about 1 hour. The reaction medium is taken up in 100 ml of dichloromethane and then washed with 3×100 ml of water. The organic phase is dried over magnesium sulfate, filtered and then concentrated to dryness under reduced pressure. The residue thus obtained is purified by flash chromatography, using a cartridge 27 mm in diameter packed with 20 g of 20-40 μm conditioned silica, and then eluted with a mixture (cyclohexane/ethyl acetate) (9/1) (v/v) at a flow rate of 8 ml/minute. The fractions between 80 and 180 ml are concentrated under reduced pressure. 0.37 g of 2-ethoxy-4-(bromomethyl)pyridine is thus obtained.

### Reference Example 1-209 b: 2-ethoxy-4-(hydroxymethyl)pyridine

0.508 g of sodium borohydride is added portionwise to a solution of 0.524 g of ethyl 2-ethoxypyridine-4-carboxylate in 5.2 ml of ethanol, under an inert atmosphere of argon at a temperature in the region of 0° C. The reaction mixture is refluxed with stirring for 3 hours and then concentrated to dryness under reduced pressure. The residue thus obtained is taken up in 50 ml of dichloromethane and then washed with 50 ml of water. The organic phase is dried over magnesium sulfate, filtered and then concentrated to dryness under reduced pressure. 0.310 g of 2-ethoxy-4-(hydroxymethyl)pyridine is thus obtained.

### Reference Example 1-209 c: Ethyl 2-ethoxypyridine-4-carboxylate

0.5 ml of concentrated sulfuric acid is added to a solution of 1 g of 2-fluoropyridine-4-carboxylic acid in 15 ml of ethanol. The reaction mixture is refluxed with stirring for 48 hours and then concentrated to dryness under reduced pressure. The residue thus obtained is taken up in 100 ml of water and then extracted with 3×100 ml of dichloromethane. The organic phase is dried over magnesium sulfate, filtered and then concentrated to dryness under reduced pressure. The crude product thus obtained is purified by flash chromatography using a cartridge 37 mm in diameter packed with 50 g of 20-40 μm conditioned silica, and then eluted with a mixture (dichloromethane/methanol) (90/10) (v/v), at a flow rate of 8 ml par minute. The fractions between 350 ml and 390 ml are concentrated under reduced pressure. 0.524 g of ethyl 2-ethoxypyridine-4-carboxylate is thus obtained in the form of an oil.

### Reference Example 1-210 a: 2-Ethyl-4-(bromomethyl)pyridine

A solution of 0.3 g of 2-ethyl-4-(hydroxymethyl)pyridine in 3.5 ml of dichloromethane is added dropwise to a solution of 1.25 g of dibromotriphenylphosphorane in 4 ml of dichloromethane, under an inert atmosphere of argon at a temperature in the region of 0° C. The mixture is stirred to a temperature in the region of 20° C. over about 1 hour. The reaction medium is taken up in 50 ml of dichloromethane and then washed with 3×50 ml of water. The organic phase is dried over magnesium sulfate, filtered and then concentrated to dryness under reduced pressure. The residue thus obtained is purified by flash chromatography using a cartridge 20 mm in diameter packed with 10 g of 20-40 μm conditioned silica, and then eluted with a mixture (cyclohexane/ethyl acetate) (9/1) (v/v), at a flow rate of 8 ml/minute. The fractions between 80 and 120 ml are concentrated under reduced pressure. 0.062 g of 2-ethyl-4-(bromomethyl)pyridine is thus obtained.

### Reference Example 210 b: 2-ethyl-4-(hydroxymethyl)pyridine

5.36 g of sodium borohydride are added portionwise to a solution of 5.08 g of ethyl 2-ethylpyridine-4-carboxylate in 53 ml of ethanol, under an inert atmosphere of argon at a temperature in the region of 0° C. The reaction mixture is refluxed with stirring for 3 hours and then concentrated to dryness under reduced pressure. The residue thus obtained is taken up in 500 ml of dichloromethane and then washed with 500 ml of water. The organic phase is dried over magnesium sulfate, filtered and then concentrated to dryness under reduced pressure. 3.16 g of 2-ethyl-4-(hydroxymethyl)pyridine are thus obtained.

### Reference Example 1-210 c: Ethyl 2-ethylpyridine-4-carboxylate

2.35 ml of concentrated sulfuric acid are added to a solution of 5 g of 2-ethylpyridine-4-carboxylic acid in 75 ml of ethanol. The reaction mixture is refluxed with stirring for about 64 hours and then concentrated to dryness under reduced pressure. The residue thus obtained is taken up in 500 ml of water and then extracted with 500 ml of dichloromethane. The organic phase is dried over magnesium sulfate, filtered and then concentrated to dryness under reduced pressure. 5.08 g of ethyl 2-ethylpyridine-4-carboxylate are thus obtained in the form of an oil.

### Reference Example 1-214 a: 2-bromo-4-(bromomethyl)pyridine

0.073 ml of acetic acid is added to a solution of 0.172 g of 2-bromo-4-methylpyridine in 2.5 ml of carbon tetrachloride, under an inert atmosphere of argon at a temperature in the region of 20° C. The mixture brought, with stirring, to a temperature of 50° C. 0.356 g of N-bromosuccinimide and 0.048 g of benzoyl peroxide are added successively at this temperature. The mixture is maintained at 80° C. for about 18 hours. After cooling, the reaction medium is placed on a cartridge 20 mm in diameter packed with 10 g of 20-40 m silica not conditioned beforehand, and then eluted with dichloromethane at a flow rate of 5 ml/minute. The fractions between 30 and 40 ml are concentrated to dryness under reduced pressure. 0.071 g of 2-bromo-4-(bromomethyl)pyridine is thus obtained.

### Reference Example 1-215 a: 2-Fluoro-4-(bromomethyl)pyridine

0.073 ml of acetic acid is added to a solution of 0.111 g of 2-fluoro-4-methylpyridine in 2.5 ml of carbon tetrachloride, under an inert atmosphere of argon at a temperature in the region of 20° C. The mixture is brought, with stirring, to a temperature of 50° C. 0.356 g of N-bromosuccinimide and 0.048 g of benzoyl peroxide are successively added at this temperature. The mixture is brought to 80° C. over about 16 hours. After cooling, the reaction medium is placed on a cartridge 20 mm in diameter packed with 10 g of 20-40 μm silica not conditioned beforehand, and then eluted with dichloromethane at a flow rate of 5 ml/minute. The fractions between 28 and 38 ml are concentrated to dryness under reduced pressure. 0.063 g of 2-fluoro-4-(bromomethyl)pyridine is thus obtained.

### Reference Example 1-216 a: 2-Cyano-4-(bromomethyl)pyridine

0.146 ml of acetic acid is added to a solution of 0.236 g of 2-cyano-4-methylpyridine in 5 ml of carbon tetrachloride, under an inert atmosphere of argon at a temperature in the region of 20° C. The mixture is brought, with stirring, to a temperature of 50° C. 0.712 g of N-bromosuccinimide and 0.096 g of benzoyl peroxide are successively added at this temperature. The mixture is maintained at 80° C. for about 18 hours. After cooling, the reaction medium is placed on a cartridge 27 mm in diameter packed with 25 g of 20-40 m silica not conditioned beforehand, and then eluted with dichloromethane at a flow rate of 10 ml/minute. The fractions between 140 and 175 ml are concentrated to dryness under reduced pressure. 0.097 g of 2-cyano-4-(bromomethyl)pyridine is thus obtained.

### Reference Example 1-243 a: 2,6-dibromo-4-(bromomethyl)pyridine

A solution of 0.5 g of 2,6-dibromo-4-(hydroxymethyl)pyridine in 7 ml of dichloromethane is added dropwise to a solution of 0.95 g of dibromotriphenylphosphorane in 5 ml of dichloromethane, under an inert atmosphere of argon at a temperature in the region of 0° C. The mixture is stirred to a temperature in the region of 20° C. over about 2 hours. The reaction medium is taken up in 100 ml of dichloromethane and then washed with 100 ml of water. The organic phase is dried over magnesium sulfate, filtered and then concentrated to dryness under reduced pressure. The residue thus obtained is purified by flash chromatography using a cartridge 37 mm in diameter packed with 50 g of 20-40 μm conditioned silica, and then eluted with a mixture (cyclohexane/ethyl acetate) (9/1) (v/v), at a flow rate of 8 ml/minute. The fractions between 135 and 200 ml are concentrated under reduced pressure. 0.349 g of 2,6-dibromo-4-(bromomethyl)pyridine is thus obtained.

### General Procedure for the Products of Example 1-243 to Example 1-254

A solution of 0.481 mM of anilino derivative in 0.6 ml of toluene is added to a suspension of 0.064 ml of diphosgene (0.53 mM) and 20 mg of vegetable charcoal in 1 ml of toluene, at a temperature in the region of −20° C. The mixture is stirred to a temperature in the region of 20° C., and then refluxed for 3 hours. The mixture is cooled to a temperature in the region of 20° C. and then filtered through Celite. A solution of 100 mg of methyl 2-methyl-2-[(pyrid-4-ylmethyl)amino]propanoate (0.48 mM) in 0.6 ml of toluene is added to the filtrate. The mixture thus obtained is refluxed for 16 hours and then cooled to a temperature in the region of 20° C. The precipitate is filtered off and the filtrate is concentrated under reduced pressure. The residue obtained is purified by preparative LC/MS.

### Summary Table of the Starting Anilino Derivatives and of the Products Prepared

TABLE 2
Example 1-244 through Example 1-255

| | | | Product Obtained | | | |
|---|---|---|---|---|---|---|
| Starting Anilino Derivative | | | | | | Amount |
| | Amount | Example | | Empirical | Molecular | Obtained |
| Name | (mg) | No. | Name | Formula | Weight | (mg) |
| 2-NITRO-4-(TRIFLUORO- | 106.7 | Example | 5,5-Dimethyl-3-(2-nitro-4- | C₁₈H₁₅F₃N₄O₅ | 424.34 | 54.2 |
| METHOXY)ANILINE | | 1-244 | trifluoromethoxyphenyl)- | | | |
| | | | 1-pyrid-4-yl- | | | |
| | | | methylimidazolidine-2,4- | | | |
| | | | dione 2 | | | |
| 4-(1,1,2,2- | 100.4 | Example | 5,5-Dimethyl-1-pyrid-4-yl- | C₁₉H₁₇F₄N₃O₃ | 411.36 | 32.1 |
| TETRAFLUOROETHOXY)ANILINE | | 1-245 | methyl-3-[4-(1,1,2,2- | | | |
| | | | tetrafluoroethoxy)phenyl] | | | |
| | | | imidazolidine-2,4-dione 2 | | | |
| 4-(DIFLUOROMETHOXY)ANILINE | 76.4 | Example | 3-(4-Difluoromethoxy- | C₁₈H₁₇F₂N₃O₃ | 361.35 | 3.9 |
| | | 1-246 | phenyl)-5,5-dimethyl-1- | | | |
| | | | pyrid-4-yl- | | | |
| | | | methylimidazolidine-2,4- | | | |
| | | | dione 2 | | | |
| 3-CHLORO-4-(TRIFLUORO- | 101.6 | Example | 3-(3-Chloro-4-trifluoro- | C₁₈H₁₅ClF₃N₃O₃ | 413.79 | 46.2 |
| METHOXY)ANILINE | | 1-247 | methoxyphenyl)-5,5- | | | |
| | | | dimethyl-1-pyrid-4-yl- | | | |
| | | | methylimidazolidine-2,4- | | | |
| | | | dione 2 | | | |
| 3-BROMO-4-(TRIFLUORO- | 122.9 | Example | 3-(3-Bromo-4-trifluoro- | C₁₈H₁₅BrF₃N₃O₃ | 458.24 | 43.2 |
| METHOXY)ANILINE | | 1-248 | methoxyphenyl)-5,5- | | | |
| | | | dimethyl-1-pyrid-4-yl | | | |
| | | | methylimidazolidine-2,4- | | | |
| | | | dione 2 | | | |
| 2,6-DICHLORO-4-(TRIFLUORO- | 118.1 | Example | 3-(2,6-Dichloro-4- | C₁₈H₁₄C₁₂F₃N₃O₃ | 448.23 | 19.9 |
| METHOXY)ANILINE | | 1-249 | trifluoromethoxyphenyl)- | | | |
| | | | 5,5-dimethyl-1-pyrid-4-yl- | | | |
| | | | methylimidazolidine-2,4- | | | |
| | | | dione 2 | | | |
| 3-CHLORO-4-(TRIFLUORO- | 109.3 | Example | 3-(3-Chloro-4-trifluoro- | C₁₈H₁₅ClF₃N₃O₂S | 429.85 | 33 |
| METHYLTHIO)ANILINE | | 1-250 | methylsulfanylphenyl)-5,5- | | | |
| | | | dimethyl-1-pyrid-4-yl- | | | |
| | | | methylimidazolidine-2,4- | | | |
| | | | dione 2 | | | |
| 2,2-DIFLUORO-5- | 83.1 | Example | 3-(2,2-Difluoro-1,3- | C18 H15 F2 N3 O4 | 375.33 | 7.4 |
| AMINOBENZODIOXOLE | | 1-251 | benzodioxol-5-yl)-5,5- | | | |
| | | | dimethyl-1-pyrid-4-yl | | | |
| | | | methylimidazolidine-2,4- | | | |
| | | | dione 2 | | | |
| 2-BROMO-4-(TRIFLUORO- | 122.9 | Example | 3-(2-Bromo-4-trifluoro- | C18 H15 Br F3 N3 O3 | 458.24 | 21.9 |
| METHOXY)ANILINE | | 1-252 | methoxyphenyl)-5,5- | | | |
| | | | dimethyl-1-pyrid-4-yl- | | | |
| | | | methylimidazolidine-2,4- | | | |
| | | | dione 2 | | | |
| 4-(HEPTAFLUOROPROPYL- | 140.8 | Example | 3-(4- | C20 H16 F7 N3 O2 S | 495.42 | 38.5 |
| THIO)ANILINE | | 1-253 | Heptafluoropropylsulfanyl- | | | |
| | | | phenyl)-5,5-dimethyl-1- | | | |
| | | | pyrid-4-yl- | | | |
| | | | methylimidazolidine-2,4- | | | |
| | | | dione 2 | | | |
| 2-METHYL-4-(TRIFLUORO- | 91.8 | Example | 5,5-Dimethyl-3-(2- | C19 H18 F3 N3 O3 | 393.37 | 68.8 |
| METHOXY)ANILINE | | 1-254 | methyl-4-trifluoro- | | | |
| | | | methoxyphenyl)-1-pyrid-4 | | | |
| | | | ylmethylimidazolidine-2,4- | | | |
| | | | dione 2 | | | |
| 2-CHLORO-4-(TRIFLUORO- | 101.6 | Example | 3-(2-Chloro-4-trifluoro- | C18 H15 Cl F3 N3 O3 | 413.79 | 77.6 |
| METHOXY)ANILINE | | 1-255 | methoxyphenyl)-5,5- | | | |
| | | | dimethyl-1-pyrid-4-yl- | | | |
| | | | methylimidazolidine-2,4- | | | |
| | | | dione 2 | | | |

TABLE 3
Summary Physicochemical Characteristics of Example 1-244 through Example 1-255

| | | | Description of the NMR |
|---|---|---|---|
| | | Description of the mass | spectrum |
| | | spectrum (analytical | (1H NMR spectrum |
| | Description of the mass | LC/MS) retention time, | (300 MHz, (CD3)2SO- |
| Example No. | spectrum (electron impact) | m/z | d6, δ in ppm) |
| Example 1-256 | m/z = 424 M+. | | 1.44 (broad s: 6H); |
| | m/z = 409 (M − CH3)+ | | 4.69 (broad s: 2H); |
| | m/z = 407 (M − OH)+ | | 7.41 (broad d, J = 5.5 Hz: |
| | m/z = 378 (M − NO2)+. | | 2H); 7.98 (d, J = 8.5 Hz: |
| | m/z = 147 C9H11N2+ | | 1H); 8.07 (broad |
| | m/z = 133 C7H5N2O+ | | d, J = 8.5 Hz: |
| | m/z = 92 C6H6N+ base peak | | 1H); 8.29 (d, J = 2.5 Hz: |
| | | | 1H); 8.56 (broad |
| | | | d, J = 5.5 Hz: |
| | | | 2H) |
| Example 1-257 | m/z = 411 M+. base peak | | 1.43 (s: 6H); 4.65 (s: |
| | m/z = 396 (M − CH3)+ | | 2H); 6.85 (tt, J = 51 and |
| | m/z = 294 (m/z = 396 − C2H2F4)+ | | 3 Hz: 1H); 7.46 (mt: 4H); |
| | m/z = 235 C9H5NO2F4+. | | 7.62 (dt, J = 8.5 and 2.5 Hz: |
| | m/z = 147 C9H11N2+ | | 2H); 8.56 (dd, J = 6 |
| | m/z = 133 C7H5N2O+ | | and 1.5 Hz: 2H) |
| | m/z = 92 C6H6N+ | | |
| Example 1-258 | | t = 2.64 min | |
| | | m/z = 362 (M + 1) | |
| Example 1-259 | m/z = 413 M+. | | 1.43 (s: 6H); 4.66 (s: |
| | m/z = 398 (M − CH3)+ | | 2H); 7.46 (d, J = 6 Hz: |
| | m/z = 237 C8H3NO2ClF3+. | | 2H); 7.66 (dd, J = 8.5 and |
| | m/z = 147 C9H11N2+ | | 2.5 Hz: 1H); 7.77 (broad |
| | m/z = 133 C7H5N2O+ | | dd, J = 8.5 and 1 Hz: |
| | m/z = 92 C6H6N+ base peak | | 1H); 7.93 (d, J = 2.5 Hz: |
| | | | 1H); 8.55 (broad |
| | | | d, J = 6 Hz: 2H) |
| Example 1-260 | m/z = 457 M+. | | 1.43 (s: 6H); 4.65 (s: |
| | m/z = 442 (M − CH3)+ | | 2H); 7.46 (broad d, J = 6 Hz: |
| | m/z = 281 C8H3NO2BrF3+. | | 2H); 7.68 (dd, J = 8.5 |
| | m/z = 147 C9H11N2+ | | and 2.5 Hz: 1H); |
| | m/z = 133 C7H5N2O+ | | 7.74 (broad d, J = 8.5 Hz: |
| | m/z = 92 C6H6N+ base peak | | 1H); 8.05 (d, J = 2.5 Hz: |
| | | | 1H); 8.55 (dd, J = 6 |
| | | | and |
| | | | 1.5 Hz: 2H) |
| Example 1-261 | m/z = 447 M+. | | 1.47 (s: 6H); 4.73 (s: |
| | m/z = 432 (M − CH3)+ | | 2H); 7.47 (broad d, J = 6 Hz: |
| | m/z = 412 (M − Cl)+ | | 2H); 8.00 (broad s: |
| | m/z = 271 C8H2NO2Cl2F3+. | | 2H); 8.58 (dd, J = 6 and |
| | m/z = 147 C9H11N2+ | | 1.5 Hz: 2H) |
| | m/z = 133 C7H5N2O+ | | |
| | m/z = 92 C6H6N+ base peak | | |
| Example 1-262 | m/z = 429 M+. base peak | | 1.43 (s: 6H); 4.66 (s: |
| | m/z = 414 (M − CH3)+ | | 2H); 7.46 (broad d, J = 6 Hz: |
| | m/z = 253 C8H3NOSClF3+. | | 2H); 7.70 (dd, J = 8.5 |
| | m/z = 147 C9H11N2+ | | and 2 Hz: 1H); 7.97 (d, J = 2 Hz: |
| | m/z = 133 C7H5N2O+ | | 1H); 8.05 (d, J = 8.5 Hz: |
| | m/z = 92 C6H6N+ | | 1H); 8.54 (dd, J = 6 |
| | | | and |
| | | | 1.5 Hz: 2H) |
| Example 1-263 | | t = 2.80 min | |
| | | m/z = 376 (M + 1) | |
| Example 1-264 | m/z = 457 M+. | | 1.43 (s: 3H); 1.48 (s: |
| | m/z = 414 (M − CH3)+ | | 3H); 4.68 (limit AB, J = 17 Hz: |
| | m/z = 378 (M − Br)+ base peak | | 2H); 7.42 (broad |
| | m/z = 350 (m/z = 378 − CO)+ | | d, J = 6 Hz: 2H); |
| | m/z = 281 C8H3NO2BrF3+. | | 7.66 (broad dd, J = 8.5 and |
| | m/z = 244 (m/z = 378 − C7H6N2O)+ | | 2 Hz: 1H); 7.83 (d, J = 8.5 Hz: |
| | m/z = 147 C9H11N2+ | | 1H); 8.07 (broad |
| | m/z = 133 C7H5N2O+ | | d, J = 2 Hz: 1H); |
| | m/z = 92 C6H6N+ | | 8.56 (dd, J = 6 and 1.5 Hz: |
| | | | 2H) |
| Example 1-265 | m/z = 495 M+. base peak | | 1.43 (s: 6H); 4.66 (s: |
| | m/z = 480 (M − CH3)+ | | 2H); 7.46 (broad d, J = 6 Hz: |
| | m/z = 319 C10H4NOSF7+. | | 2H); 7.71 (broad d, J = 8.5 Hz: |
| | m/z = 150 (m/z = 319 − C3F7)+ | | 2H); |
| | m/z = 147 C9H11N2+ | | 7.89 (broad d, J = 8.5 Hz: |
| | m/z = 133 C7H5N2O+ | | 2H); 8.55 (dd, J = 6 and |
| | m/z = 92 C6H6N+ | | 1.5 Hz: 2H) |
| Example 1-266 | m/z = 393 M+. base peak | | 1.43 and 1.44 (2 s: 6H in |
| | m/z = 378 (M − CH3)+ | | total); 2.21 (s: 3H); |
| | m/z = 217 C9H6NO2F3+. | | 4.65 (broad s: 2H); |
| | m/z = 147 C9H11N2+ | | 7.36 (broad d, J = 8.5 Hz: |
| | m/z = 133 C7H5N2O+ | | 1H); 7.42 (broad d, J = 6 Hz: |
| | m/z = 92 C6H6N+ | | 2H); 7.45 (broad s: |
| | | | 1H); 7.55 (d, J = 8.5 Hz: |
| | | | 1H); 8.55 (dd, J = 6 and |
| | | | 1.5 Hz: 2H) |
| Example 1-267 | m/z = 413 M+. | | 1.43 (s: 3H); 1.48 (s: |
| | m/z = 398 (M − CH3)+ | | 3H); 4.67 (broad s: 2H); |
| | m/z = 378 (M − Cl)+ base peak | | 7.42 (broad d, J = 6 Hz: |
| | m/z = 350 (m/z = 378 − CO)+ | | 2H); 7.62 (ddd, J = 8.5-2.5 |
| | m/z = 237 C8H3NO2ClF3+. | | and 1 Hz: 1H); |
| | m/z = 147 C9H11N2+ | | 7.85 (d, J = 8.5 Hz: |
| | m/z = 133 C7H5N2O+ | | 1H); 7.88 (broad |
| | m/z = 92 C6H6N+ | | d, J = 2.5 Hz: |
| | | | 1H); 8.57 (dd, J = 6 |
| | | | and |
| | | | 1.5 Hz: 2H) |

The products of Example 1-268 to Example 1-275 were obtained according to the procedure described above.

### Example 1-268: Methyl 5-(5-hydroxy-4,4-dimethyl-2-oxo-3-quinolin-4-ylmethyl-imidazolidin-1-yl)-2-trifluoromethoxybenzoate

1H NMR (DMSO)

(P-31800-112-1): 1.47 ppm (s, 6H); 3.89 ppm (s, 3H); 5.15 ppm (s, 2H); from 7.65 to 7.73 ppm (m, 3H); 7.82 ppm (bt, J=8.5 Hz, 1H); 7.92 ppm (dd, J=2.5-8.5 Hz, 1H); 8.07 ppm (bd, J=8.5 Hz, 1H); 8.17 ppm (d, J=2.5 Hz, 1H); 8.26 ppm (bd, J=8.5 Hz, 1H); 8.86 ppm (d, J=4.5 Hz, 1H)

LC/MS: MH⁺=488

### Example 1-269: 3-(3-Bromo-4-trifluoromethoxyphenyl)-5,5-dimethyl-1-quinolin-4-ylmethylimidazolidine-2,4-dione

1H NMR (DMSO)

(P-31800-101-1): 1.45 ppm (s, 6H); 5.15 ppm (s, 2H); from 7.62 to 7.72 ppm (m, 4H); 7.80 ppm (bt, J=8.5 Hz, 1H); 8.08 ppm (m, 2H); 8.26 ppm (bd, J=8.5 Hz, 1H); 8.86 ppm (d, J=4.5 Hz).

LC/SM: MH+=508

### Example 1-270: 3-(3-Chloro-4-trifluoromethoxyphenyl)-5,5-dimethyl-1-quinolin-4-ylmethylimidazoline-2,4-dione

1H NMR (DMSO):

P-31800-105-1 NMR No. 6354R: 1.44 ppm (s, 6H); 5.14 ppm (s, 2H); from 7.61 to 7.84 ppm (m, 5H); 7.94 ppm (d, J=2.5 Hz, 1H); 8.07 ppm (bd, J=8.5 Hz, 1H); 8.24 ppm (bd, J=8.5 Hz, 1H); 8.83 ppm (d, J=4.5 Hz, 1H).

LC/MS: MH+=464

### Example 1-271: 5-(4,4-Dimethyl-2,5-dioxo-3-quinolin-4-ylmethylimidazolidin-1-yl)-2-trifluoromethylbenzoic acid

1H NMR (DMSO)

(P-29798-111-1): 1.47 ppm (s, 6H); 5.15 ppm (s, 2H); from 7.62 to 7.72 ppm (m, 3H); 7.81 ppm (bt, J=8.5 Hz, 1H); 7.88 ppm (dd, J=2.5-8.5, 1H); 8.08 ppm (bt, J=8.5 Hz, 1H); 8.15 ppm (d, J=2.5 Hz, 1H); 8.26 ppm (bd, J=8.5 Hz, 1H); 8.87 ppm (d, J=4.5 Hz, 1H); 13.5 ppm (bs, 1H).

LC/MS: MH⁺=474

### Example 1-272: 5,5-Dimethyl-3-{3-[4-(4-methylpiperazin-1-yl)butyryl]-4-trifluoro-methoxyphenyl}-1-quinolin-4-ylmethylimidazolidine-2,4-dione

1H NMR (DMSO)

(P-29798-112-1): 1.51 ppm (s, 6H); 1.97 ppm (m, 2H); 2.82 ppm (s, 3H); from 3.0 to 4.0 ppm (broad multiplet, 12H); 5.35 ppm (s, 2H); 7.63 ppm (dq, J=1.5-8.5 Hz, 1H); 7.75 ppm (dd, J=2.5-8.5 Hz, 1H); 7.82 ppm (d, J=2.5 Hz, 1H); 7.94 ppm (bt, J=8.5 Hz, 1H); 8.10 ppm (m, 2H); 8.39 ppm (bd, J=8.5 Hz, 1H); 8.51 ppm (bd, J=8.5 Hz, 1H); 8.72 ppm (bt, J=5.5 Hz, 1H); 9.15 ppm (d, J=5 Hz, 1H); 11.97 ppm (bs, 1H).

MS/EI: M+=612+

### Example 1-273: 5-(5-Hydroxy-4,4-dimethyl-2-oxo-3-quinolin-4-ylmethylimidazolidin-1-yl)-2-trifluoromethoxybenzoic acid

The following are introduced into a 30 ml round-bottomed flask equipped with a magnetic stirrer and a septum on which is mounted a nitrogen inlet:

200 mg (0.41 mmol) of ester in 2 ml of ether;

8 mg (0.205 mmol) of LiAlH₄, i.e., 8 ml of a 1M solution in 2 ml of THF.

The reaction medium is stirred at room temperature for 24 hours.

By TLC; 8/2 CH₂Cl₂/EtOAc: a product more polar than the starting material is formed, but starting material remains.

A further 0.5 equivalent of LiAlH₄ is added and the mixture is stirred for 5 hours. A small amount of starting material still remains, but the reaction mixture is worked up.

The reaction medium is hydrolyzed by adding water thereto, and is extracted with 3 times 10 ml of ethyl acetate. The organic fractions are combined and dried over magnesium sulfate, and the EtOAc is evaporated off.

The crude reaction product is purified using a CH₂Cl₂/EtOAc eluent mixture (9/1).

60 mg of alcohol are thus obtained.

1H NMR (DMSO): 1.19 ppm (s, 3H); 1.24 ppm (s, 3H); 3.86 ppm (s, 3H); 4.92 ppm (AB system, J=18 Hz, 2H); 5.36 ppm (s, 1H); 7.51 ppm (dm, J=9.0 Hz, 1H); 7.58 ppm (d, J=4.5 Hz, 1H); 7.66 (m, 1H); 7.80 ppm (m, 1H); 8.00 ppm (dd, J=2.5-9.0 Hz, 1H); 8.06 ppm (dd, J=1.5-8.5 Hz, 1H); 8.28 ppm (dd, J=1.5-8.5 Hz, 1H); 8.33 ppm (d, J=2.5 Hz, 1H); 8.84 ppm (df, J=4.5 Hz, 1H).

LC/MS: MH+=489

### Example 1-274: 4,4-dimethyl-3-quinolin-4-ylmethyl-5-thioxo-1-(4-trifluoro-methoxyphenyl)imidazolidin-2-one

The following reaction mixture:

317 mg (0.739×10⁻³ mol) of hydantoin SB 31051-139
78 mg (0.19×10⁻³ mol) of Lawesson's reagent
1 cm³ of toluene is refluxed for 9 hours and then cooled, filtered and concentrated under reduced pressure. A yellow foam is recovered, and is purified by chromatography several times on silica, with chloroform/acetone eluent (97/3). 231 mg of pure yellow resin are obtained: Rf=0.38 Th=329 mg Yield=70%

1H NMR (DMSO) ppm

(P-31051-146-2): 1.57 ppm (s, 6H); 5.24 ppm (s, 2H); 7.57 ppm (m, 2H); 7.63 ppm (d, J=4.5 Hz, 1H); 7.70 ppm (m, 3H); 7.81 ppm (bt, J=8.5 Hz, 1H); 8.08 ppm (dd, J=1.5-8.5 Hz, 1H); 8.26 ppm (dd, J=1.5-8.5 Hz, 1H); 8.86 ppm (d, J=4.5 Hz, 1H).

LC/SM: MH+=446

### Example 1-275: 5,5-dimethyl-1-quinolin-4-ylmethyl-2-thioxo-3-(4-trifluoro-methoxyphenyl)imidazolidin-4-one

The following reaction mixture:

0.6 g (mmol) of quinoline derivative
0.918 g (mmol) of thioisocyanate
12 cm³ of anhydrous THF

### 0.2 cm³ of TEA

is stirred at room temperature for 20 hours.

The mixture is concentrated under reduced pressure and then purified on a 25 g AIT column, eluent 95/5 DCM/acetone.

The product of Rf=0.12 is recovered, i.e., 266 mg

Yield=26%

1H NMR (DMSO)

(P-31051-160-2): 1.55 ppm (s, 6H); 5.56 ppm (s, 2H); 7.55 ppm (m, 3H); 7.69 ppm (m, 3H); 7.82 ppm (bt, J=8.5 Hz, 1H); 8.08 ppm (dd, J=1.5-8.5, 1H); 8.28 ppm (dd, J=1.5-8.5, 1H); 8.83 ppm (d, J=4.5 Hz, 1H).

MS: M⁺=445⁺

The products of Example 1-276 to Example 1-344 are described in Table 1 below: these products are prepared as indicated in the experimental section of the present patent application and especially as indicated for the products of Example 1-70 to Example 1-200, and thus constitute Example 1-276 to Example 1-344 of the present invention.

TABLE 4
Example 1-264 through Example 1-332

| Examples | Structure |
|---|---|
| Example 1-276 | 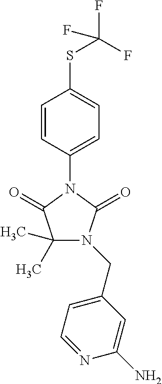 [SMILES: CC1(C)C(=O)N(c2ccc(SC(F)(F)F)cc2)C(=O)N1Cc1ccnc(N)c1] |
| Example 1-277 | 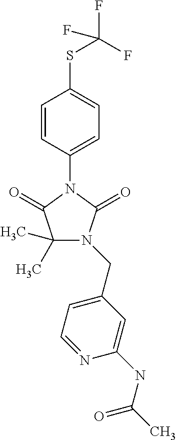 [SMILES: CC(=O)Nc1cc(CN2C(=O)N(c3ccc(SC(F)(F)F)cc3)C(=O)C2(C)C)ccn1] |
| Example 1-278 | 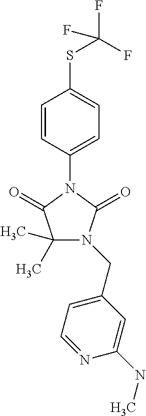 [SMILES: CNc1cc(CN2C(=O)N(c3ccc(SC(F)(F)F)cc3)C(=O)C2(C)C)ccn1] |
| Example 1-279 | 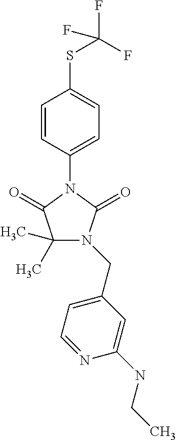 [SMILES: CCNc1cc(CN2C(=O)N(c3ccc(SC(F)(F)F)cc3)C(=O)C2(C)C)ccn1] |
| Example 1-280 | 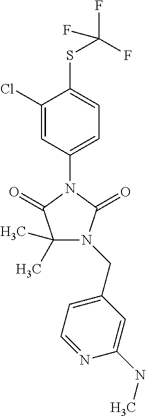 [SMILES: CNc1cc(CN2C(=O)N(c3ccc(SC(F)(F)F)c(Cl)c3)C(=O)C2(C)C)ccn1] |
| Example 1-281 | 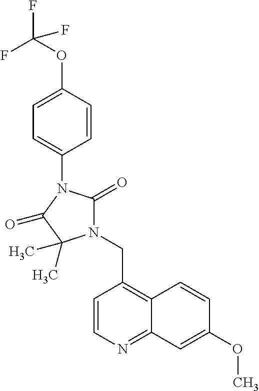 [SMILES: COc1ccc2c(CN3C(=O)N(c4ccc(OC(F)(F)F)cc4)C(=O)C3(C)C)ccnc2c1] |
| Example 1-282 | 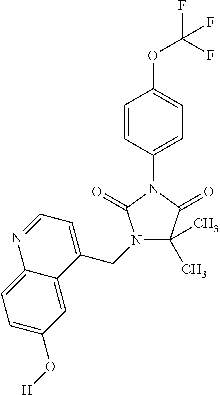 [SMILES: CC1(C)C(=O)N(c2ccc(OC(F)(F)F)cc2)C(=O)N1Cc1ccnc2ccc(O)cc12] |
| Example 1-283 | 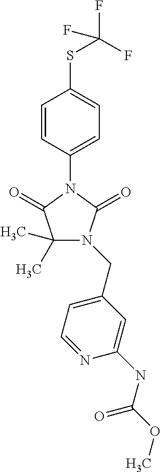 [SMILES: COC(=O)Nc1cc(CN2C(=O)N(c3ccc(SC(F)(F)F)cc3)C(=O)C2(C)C)ccn1] |
| Example 1-284 | 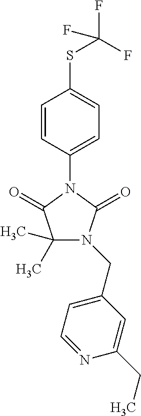 [SMILES: CCc1cc(CN2C(=O)N(c3ccc(SC(F)(F)F)cc3)C(=O)C2(C)C)ccn1] |
| Example 1-285 | 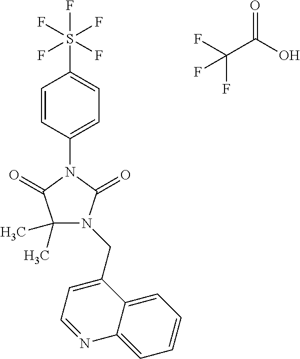 [SMILES: CC1(C)C(=O)N(c2ccc(S(F)(F)(F)(F)F)cc2)C(=O)N1Cc1ccnc2ccccc12 ; O=C(O)C(F)(F)F] |
| Example 1-286 | 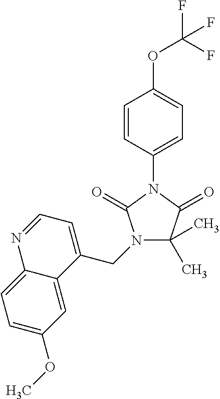 [SMILES: COc1ccc2nccc(CN3C(=O)N(c4ccc(OC(F)(F)F)cc4)C(=O)C3(C)C)c2c1] |
| Example 1-287 | 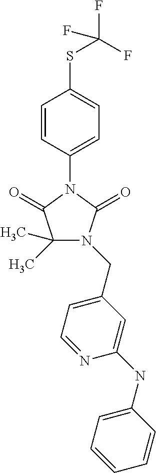 [SMILES: CC1(C)C(=O)N(c2ccc(SC(F)(F)F)cc2)C(=O)N1Cc1ccnc(Nc2ccccc2)c1] |
| Example 1-288 | 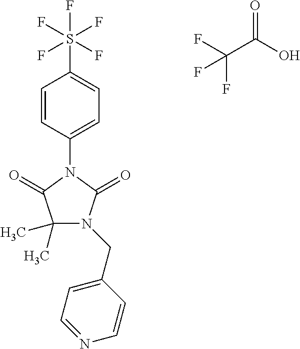 [SMILES: CC1(C)C(=O)N(c2ccc(S(F)(F)(F)(F)F)cc2)C(=O)N1Cc1ccncc1 ; O=C(O)C(F)(F)F] |
| Example 1-289 | 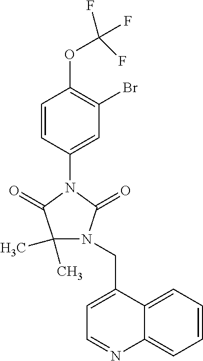 [SMILES: CC1(C)C(=O)N(c2ccc(OC(F)(F)F)c(Br)c2)C(=O)N1Cc1ccnc2ccccc12] |
| Example 1-290 | 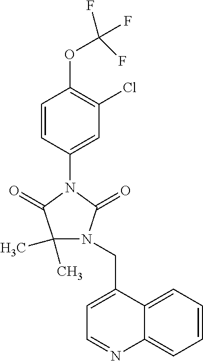 [SMILES: CC1(C)C(=O)N(c2ccc(OC(F)(F)F)c(Cl)c2)C(=O)N1Cc1ccnc2ccccc12] |
| Example 1-291 | 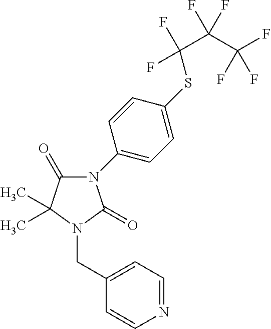 [SMILES: CC1(C)C(=O)N(c2ccc(SC(F)(F)C(F)(F)C(F)(F)F)cc2)C(=O)N1Cc1ccncc1] |
| Example 1-292 | 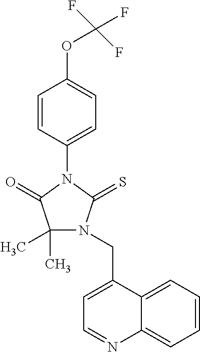 [SMILES: CC1(C)C(=O)N(c2ccc(OC(F)(F)F)cc2)C(=S)N1Cc1ccnc2ccccc12] |
| Example 1-293 | 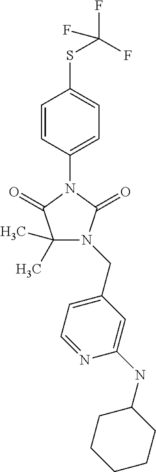 [SMILES: CC1(C)C(=O)N(c2ccc(SC(F)(F)F)cc2)C(=O)N1Cc1ccnc(NC2CCCCC2)c1] |
| Example 1-294 | 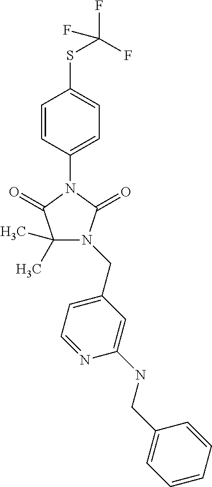 [SMILES: CC1(C)C(=O)N(c2ccc(SC(F)(F)F)cc2)C(=O)N1Cc1ccnc(NCc2ccccc2)c1] |
| Example 1-295 | 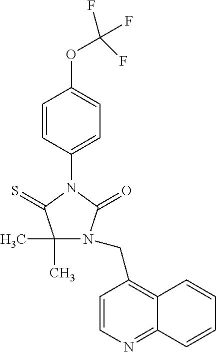 [SMILES: CC1(C)C(=S)N(c2ccc(OC(F)(F)F)cc2)C(=O)N1Cc1ccnc2ccccc12] |
| Example 1-296 | 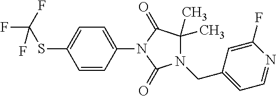 [SMILES: CC1(C)C(=O)N(c2ccc(SC(F)(F)F)cc2)C(=O)N1Cc1ccnc(F)c1] |
| Example 1-297 | 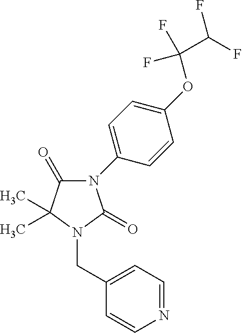 [SMILES: CC1(C)C(=O)N(c2ccc(OC(F)(F)C(F)F)cc2)C(=O)N1Cc1ccncc1] |
| Example 1-298 | 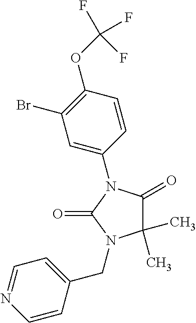 [SMILES: CC1(C)C(=O)N(c2ccc(OC(F)(F)F)c(Br)c2)C(=O)N1Cc1ccncc1] |
| Example 1-299 | 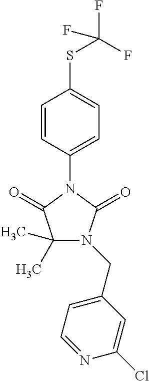 [SMILES: CC1(C)C(=O)N(c2ccc(SC(F)(F)F)cc2)C(=O)N1Cc1ccnc(Cl)c1] |
| Example 1-300 | 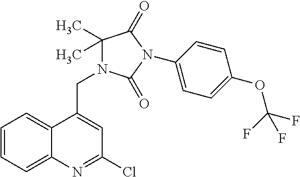 [SMILES: CC1(C)C(=O)N(c2ccc(OC(F)(F)F)cc2)C(=O)N1Cc1cc(Cl)nc2ccccc12] |
| Example 1-301 | 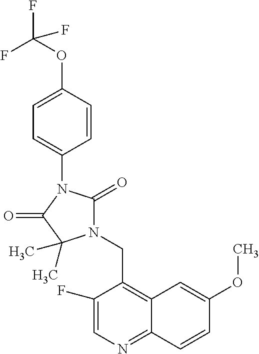 [SMILES: COc1ccc2ncc(F)c(CN3C(=O)N(c4ccc(OC(F)(F)F)cc4)C(=O)C3(C)C)c2c1] |
| Example 1-302 | 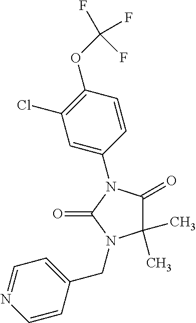 [SMILES: CC1(C)C(=O)N(c2ccc(OC(F)(F)F)c(Cl)c2)C(=O)N1Cc1ccncc1] |
| Example 1-303 | 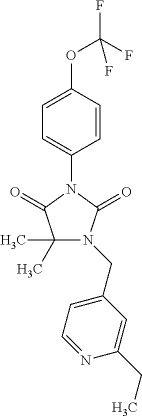 [SMILES: CCc1cc(CN2C(=O)N(c3ccc(OC(F)(F)F)cc3)C(=O)C2(C)C)ccn1] |
| Example 1-304 | 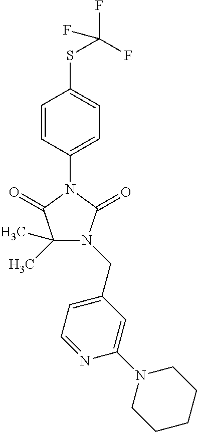 [SMILES: CC1(C)C(=O)N(c2ccc(SC(F)(F)F)cc2)C(=O)N1Cc1ccnc(N2CCCCC2)c1] |
| Example 1-305 | 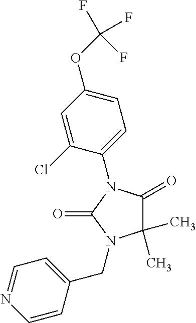 [SMILES: CC1(C)C(=O)N(c2ccc(OC(F)(F)F)cc2Cl)C(=O)N1Cc1ccncc1] |
| Example 1-306 | 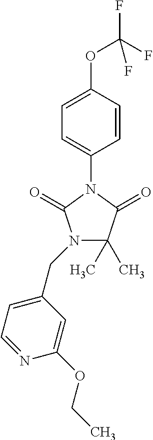 [SMILES: CCOc1cc(CN2C(=O)N(c3ccc(OC(F)(F)F)cc3)C(=O)C2(C)C)ccn1] |
| Example 1-307 | 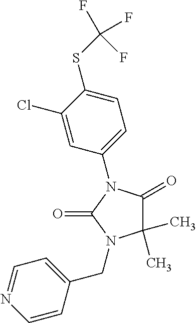 [SMILES: CC1(C)C(=O)N(c2ccc(SC(F)(F)F)c(Cl)c2)C(=O)N1Cc1ccncc1] |
| Example 1-308 | 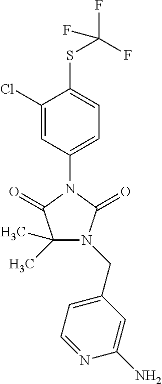 [SMILES: CC1(C)C(=O)N(c2ccc(SC(F)(F)F)c(Cl)c2)C(=O)N1Cc1ccnc(N)c1] |
| Example 1-309 | 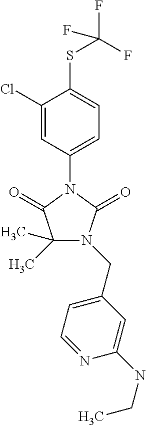 [SMILES: CCNc1cc(CN2C(=O)N(c3ccc(SC(F)(F)F)c(Cl)c3)C(=O)C2(C)C)ccn1] |
| Example 1-310 | 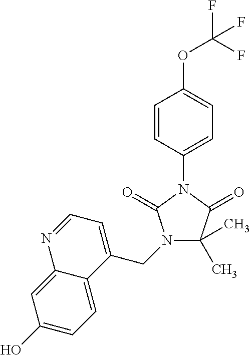 [SMILES: CC1(C)C(=O)N(c2ccc(OC(F)(F)F)cc2)C(=O)N1Cc1ccnc2cc(O)ccc12] |
| Example 1-311 | 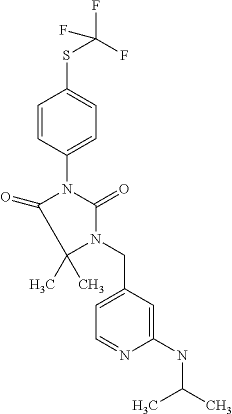 [SMILES: CC(C)Nc1cc(CN2C(=O)N(c3ccc(SC(F)(F)F)cc3)C(=O)C2(C)C)ccn1] |
| Example 1-312 | 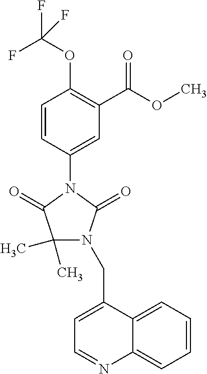 [SMILES: COC(=O)c1cc(N2C(=O)N(Cc3ccnc4ccccc34)C(C)(C)C2=O)ccc1OC(F)(F)F] |
| Example 1-313 | 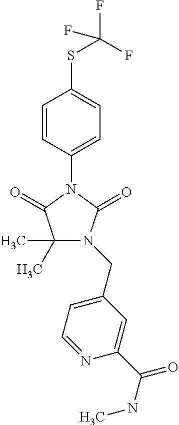 [SMILES: CNC(=O)c1cc(CN2C(=O)N(c3ccc(SC(F)(F)F)cc3)C(=O)C2(C)C)ccn1] |
| Example 1-314 | 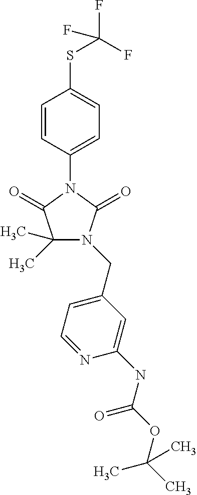 [SMILES: CC(C)(C)OC(=O)Nc1cc(CN2C(=O)N(c3ccc(SC(F)(F)F)cc3)C(=O)C2(C)C)ccn1] |
| Example 1-315 | 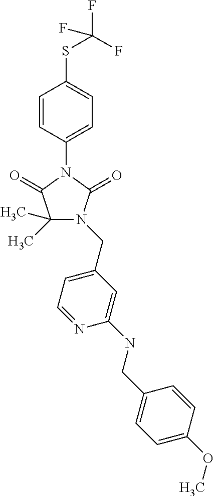 [SMILES: COc1ccc(CNc2cc(CN3C(=O)N(c4ccc(SC(F)(F)F)cc4)C(=O)C3(C)C)ccn2)cc1] |
| Example 1-316 | 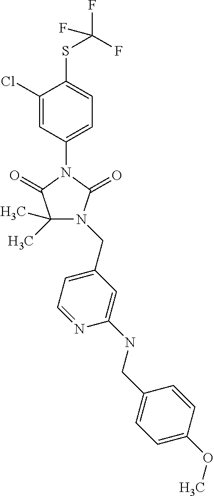 [SMILES: COc1ccc(CNc2cc(CN3C(=O)N(c4ccc(SC(F)(F)F)c(Cl)c4)C(=O)C3(C)C)ccn2)cc1] |
| Example 1-317 | 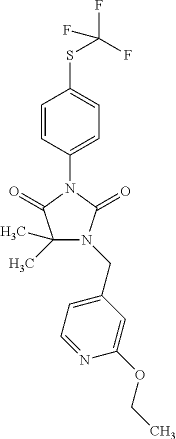 [SMILES: CCOc1cc(CN2C(=O)N(c3ccc(SC(F)(F)F)cc3)C(=O)C2(C)C)ccn1] |
| Example 1-318 | 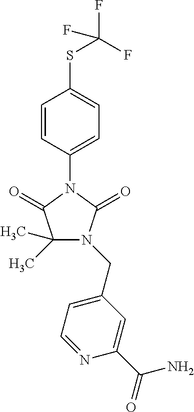 [SMILES: CC1(C)C(=O)N(c2ccc(SC(F)(F)F)cc2)C(=O)N1Cc1ccnc(C(N)=O)c1] |
| Example 1-319 | 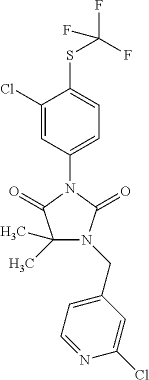 [SMILES: CC1(C)C(=O)N(c2ccc(SC(F)(F)F)c(Cl)c2)C(=O)N1Cc1ccnc(Cl)c1] |
| Example 1-320 | 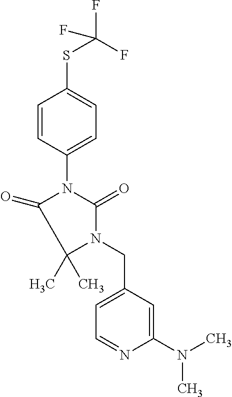 [SMILES: CN(C)c1cc(CN2C(=O)N(c3ccc(SC(F)(F)F)cc3)C(=O)C2(C)C)ccn1] |
| Example 1-321 | 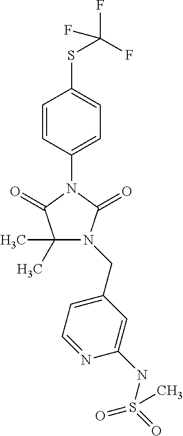 [SMILES: CC1(C)C(=O)N(c2ccc(SC(F)(F)F)cc2)C(=O)N1Cc1ccnc(NS(C)(=O)=O)c1] |
| Example 1-322 | 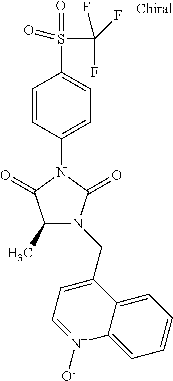 [SMILES: C[C@H]1C(=O)N(c2ccc(S(=O)(=O)C(F)(F)F)cc2)C(=O)N1Cc1cc[n+]([O-])c2ccccc12] |
| Example 1-323 | 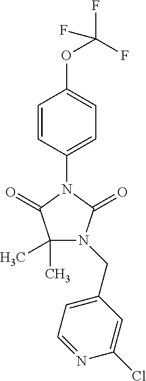 [SMILES: CC1(C)C(=O)N(c2ccc(OC(F)(F)F)cc2)C(=O)N1Cc1ccnc(Cl)c1] |
| Example 1-324 | 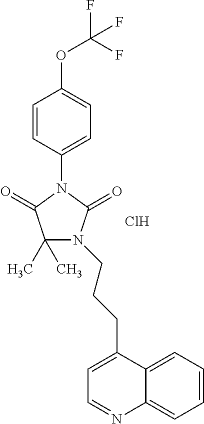 [SMILES: CC1(C)C(=O)N(c2ccc(OC(F)(F)F)cc2)C(=O)N1CCCc1ccnc2ccccc12 ; Cl] |
| Example 1-325 | 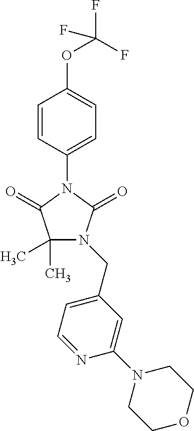 [SMILES: CC1(C)C(=O)N(c2ccc(OC(F)(F)F)cc2)C(=O)N1Cc1ccnc(N2CCOCC2)c1] |
| Example 1-326 | 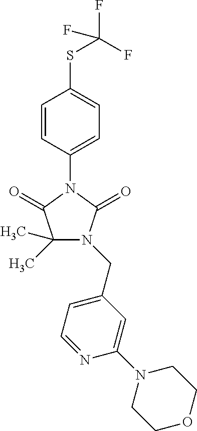 [SMILES: CC1(C)C(=O)N(c2ccc(SC(F)(F)F)cc2)C(=O)N1Cc1ccnc(N2CCOCC2)c1] |
| Example 1-327 | 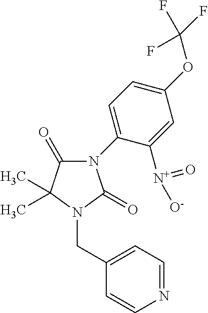 [SMILES: CC1(C)C(=O)N(c2ccc(OC(F)(F)F)cc2[N+](=O)[O-])C(=O)N1Cc1ccncc1] |
| Example 1-328 | 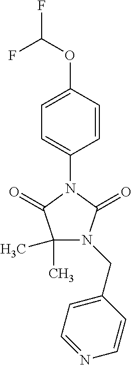 [SMILES: CC1(C)C(=O)N(c2ccc(OC(F)F)cc2)C(=O)N1Cc1ccncc1] |
| Example 1-329 | 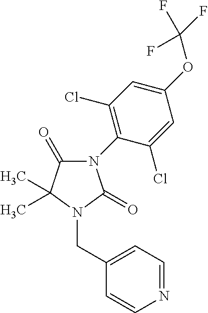 [SMILES: CC1(C)C(=O)N(c2c(Cl)cc(OC(F)(F)F)cc2Cl)C(=O)N1Cc1ccncc1] |
| Example 1-330 | 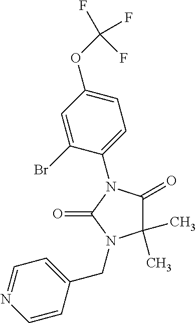 [SMILES: CC1(C)C(=O)N(c2ccc(OC(F)(F)F)cc2Br)C(=O)N1Cc1ccncc1] |
| Example 1-331 | 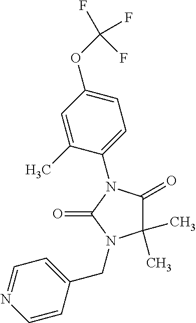 [SMILES: Cc1cc(OC(F)(F)F)ccc1N1C(=O)N(Cc2ccncc2)C(C)(C)C1=O] |
| Example 1-332 | 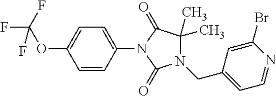 [SMILES: CC1(C)C(=O)N(c2ccc(OC(F)(F)F)cc2)C(=O)N1Cc1ccnc(Br)c1] |
| Example 1-333 | 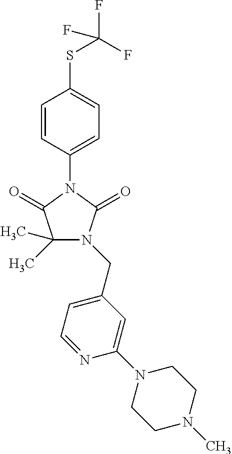 [SMILES: CN1CCN(c2cc(CN3C(=O)N(c4ccc(SC(F)(F)F)cc4)C(=O)C3(C)C)ccn2)CC1] |
| Example 1-334 | 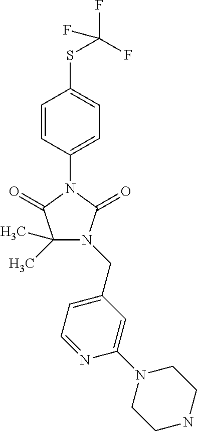 [SMILES: CC1(C)C(=O)N(c2ccc(SC(F)(F)F)cc2)C(=O)N1Cc1ccnc(N2CCNCC2)c1] |
| Example 1-335 | 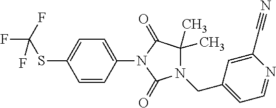 [SMILES: CC1(C)C(=O)N(c2ccc(SC(F)(F)F)cc2)C(=O)N1Cc1ccnc(C#N)c1] |
| Example 1-336 | 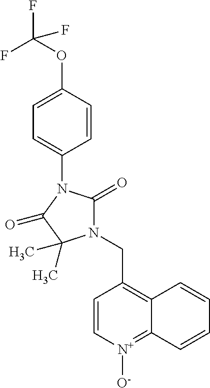 [SMILES: CC1(C)C(=O)N(c2ccc(OC(F)(F)F)cc2)C(=O)N1Cc1cc[n+]([O-])c2ccccc12] |
| Example 1-337 | 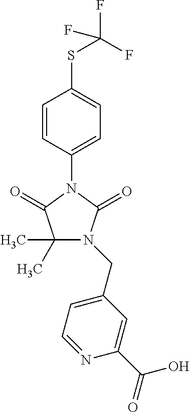 [SMILES: CC1(C)C(=O)N(c2ccc(SC(F)(F)F)cc2)C(=O)N1Cc1ccnc(C(=O)O)c1] |
| Example 1-338 | 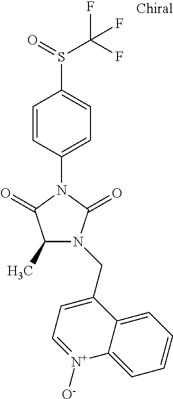 [SMILES: C[C@H]1C(=O)N(c2ccc(S(=O)C(F)(F)F)cc2)C(=O)N1Cc1cc[n+]([O-])c2ccccc12] |
| Example 1-339 | 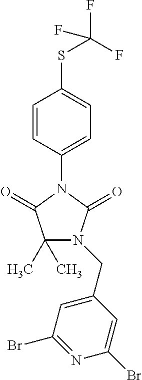 [SMILES: CC1(C)C(=O)N(c2ccc(SC(F)(F)F)cc2)C(=O)N1Cc1cc(Br)nc(Br)c1] |
| Example 1-340 | 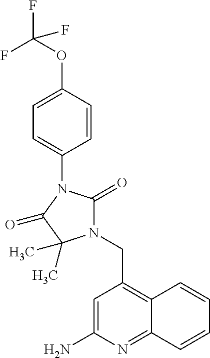 [SMILES: CC1(C)C(=O)N(c2ccc(OC(F)(F)F)cc2)C(=O)N1Cc1cc(N)nc2ccccc12] |
| Example 1-341 | 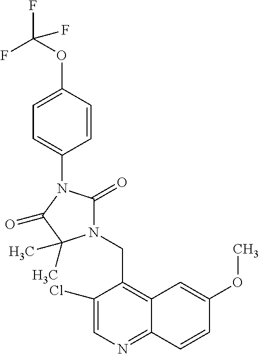 [SMILES: COc1ccc2ncc(Cl)c(CN3C(=O)N(c4ccc(OC(F)(F)F)cc4)C(=O)C3(C)C)c2c1] |
| Example 1-342 | 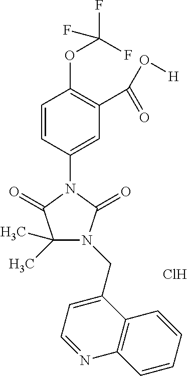 [SMILES: CC1(C)C(=O)N(c2ccc(OC(F)(F)F)c(C(=O)O)c2)C(=O)N1Cc1ccnc2ccccc12 ; Cl] |
| Example 1-343 | 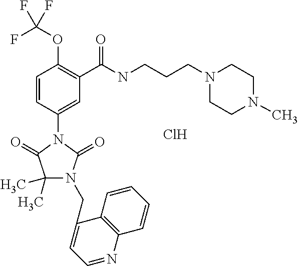 [SMILES: CN1CCN(CCCNC(=O)c2cc(N3C(=O)N(Cc4ccnc5ccccc45)C(C)(C)C3=O)ccc2OC(F)(F)F)CC1 ; Cl] |
| Example 1-344 | 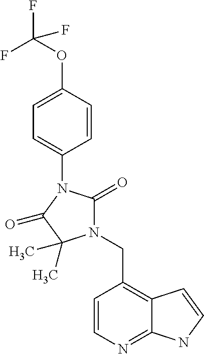 [SMILES: CC1(C)C(=O)N(c2ccc(OC(F)(F)F)cc2)C(=O)N1Cc1ccnc2[nH]ccc12] |

### Example 2: In Vitro Studies

### Example 2-1: Cell-Based TrkA Activity Assay

Nerve growth factor inhibition activity was tested using the CellSensor® TrkA-NFAT-bla CHO-K1 cell line as described by the manufacturer (Invitrogen—Carlsbad, Calif.). The CHO-K1 cells contain a beta-lactamase reporter gene under control of the NFAT Response Element that has been stably integrated into the cells along with TrkA. This cell line is a clonal population isolated by flow cytometry that has been tested for robust assay performance by assessing a variety of assay parameters. TrkA-NFAT-bla CHO-K1 cells express beta-lactamase upon stimulation with nerve growth factor 2.5 s (NGF 2.5 s). Briefly, cells were plated overnight. A dose curve of inhibitor was added for 30 minutes, followed by 5 hours of stimulation with NGF 2.5 s. FRET peptide was then added for 2 hours and the assay plate was read on a fluorescence plate reader. Results are shown in FIG. 1.

1. A method of treating inflammatory diseases and/or defects of bone metabolism in a patient in need thereof comprising administering to the patient a cyclic urea compound comprising the structure of Formula (I): 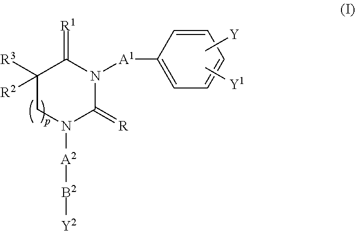
wherein:
p is 0, 1 or 2;
R and R¹, which may be identical or different, are O or NH;
R² and R³, which may be identical or different, are hydrogen, alkyl, alkenyl, alkynyl, cycloalkyl, aryl and heteroaryl that are optionally substituted, or R² and R³ taken together with the carbon atom to which they are attached form 3- to 10-membered carbocyclyl that is optionally being substituted or 3- to 10-membered heterocyclyl containing one or more hetero atoms chosen from O, S, N and NR⁷ that is optionally being substituted;
A¹ is single bond, alkyl, alkenyl or alkynyl;
Y and Y¹, which may be identical or different, are such that one of Y and Y¹ is —OCF₃, —O—F₂—CHF₂, —O—CHF₂, —O—CH₂—CF₃, —SO₂NR⁵R⁶, —SF₅ and —S(O)ₙ-alkyl and the other of Y and Y¹ is —OCF₃, —O—F₂—CHF₂, —O—CHF₂, —O—CH₂—CF₃, SO₂NR⁵R⁶, —SF₅, —S(O)ₙ-alkyl, hydrogen, halogen, hydroxyl, alkoxy, nitro, —CN, —NR⁵R⁶, optionally substituted alkyl, optionally substituted aryl, optionally substituted heteroaryl, —CF₃, —O-alkenyl, —O-alkynyl, —O-cycloalkyl, —S(O)ₙ-alkenyl, —S(O)ₙ-alkynyl, —S(O)ₙ-cycloalkyl, —CONR⁵R⁶, or free, salified or esterified carboxyl, or the 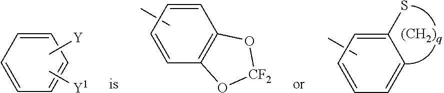
that is optionally substituted with one or more alkyl that are optionally substituted, wherein
q is 2, 3 or 4;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
A², which may be identical to or different from A¹, is defined as A¹ or is CO or SO₂,
B² is saturated or unsaturated, 3- to 10-membered monocyclic or bicyclic heterocyclyl containing one or more hetero atoms chosen from O, S, N and NR⁷ that is optionally substituted with one or more identical or different substituents defined as Y², wherein
R⁷ is hydrogen, alkyl, cycloalkyl, phenyl, acyl, —S(O)₂Alk, —S(O)₂Aryl, —S(O)₂heteroaryl or —S(O)₂NR⁵R⁶,
Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹⁰—CS—NR⁵R⁶ or —NR¹⁰—COOR¹³, all of which are optionally substituted;
all the alkyl, alkenyl, alkynyl and alkoxy above are linear or branched and contain not more than 6 carbon atoms,
all the cycloalkyl and heterocyclyl above contain not more than 7 carbon atoms,
all the aryl and heteroaryl above contain not more than 10 carbon atoms,
all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²,
all the aryl and heteroaryl above are optionally substituted with one or more substituents selected from the group consisting of alkyl, alkoxy and alkylenedioxy,
all the cyclic radicals above, and also the ring formed by R⁵ and R⁶ with the nitrogen atom to which they are attached, are optionally substituted with one or more substituents selected from the group consisting of oxo and thioxo,
n is 0 to 2, and wherein
R⁸ is alkyl, alkenyl, cycloalkyl, cycloalkylalkyl, heterocyclyl, heterocyclylalkyl, aryl, arylalkyl, heteroaryl and heteroarylalkyl,
R⁹ is defined as R⁸ or is hydrogen,
R¹⁰ is hydrogen or alkyl,
R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl, or R¹¹ and R¹² taken together with the nitrogen atom to which they are attached form 5- to 7-membered cyclic radical containing one or more hetero atoms chosen from O, S, N and NR⁷, preferably a cyclic amine, and
R¹³, which may be identical to or different to R⁵ or R⁶, is defined as R⁵ or R⁶, or
a racemic, enantiomeric or diastereoisomeric isomer form of the compound of Formula (I), addition salt with mineral or organic acid or with mineral or organic base thereof, with the proviso:
a) when p is 0, R and R¹ are oxygen, A¹ is single bond or alkyl, Y and Y¹, which may be identical or different, are at least one is —OCF₃ or —S-alk, A² is single bond or alkyl and B² is an optionally substituted heterocyclyl, then R² and R³ are not one hydrogen and the other imidazolylalkyl;
b) when p is 0, R and R¹ are oxygen, A¹ is single bond or alkyl, Y and Y¹, which may be identical or different, are at least one is —OCF₃, —SO-Alk, —S(O)₂-alk or —SO₂NH₂, A² is CH₂ and B² is an optionally substituted heterocyclyl, then R² and R³ are not one hydrogen and the other alkyl optionally interrupted with O, S or N-alk; always substituted with a hydroxamate (—CO—NHOH);
c) when p is 0, R and R¹ are oxygen, A¹ is a single bond or alkyl, Y and Y¹, which may be identical or different, are at least one is —S(O)ₙ-alk, A² is single bond and B² is an optionally substituted 5- or 6-membered aromatic heterocyclyl, then R² and R³ are not selected from the group consisting of hydrogen, alkyl, arylalkyl, aryl and heteroaryl; or
d) when p is 0 to 2, R and R¹ are oxygen, A¹ is single bond, Y and Y¹, which may be identical or different, are one is —SO₂Alk or SO₂NH₂ and the other is NR⁵R⁶, A² is single bond or alkylene and B² is optionally substituted 5- to 10-membered heterocyclyl, then R² and R³ are not both hydrogen.

2. The method according to claim 1 comprising administering to the patient a cyclic urea compound comprising the structure of Formula (II): 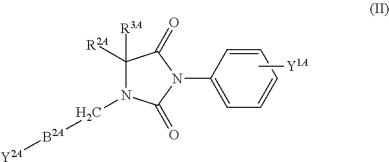
wherein:
Y^{1A} is —OCF₃, —S(O)ₙ—CF₃ and —SO₂CHF₂;
B^{2A} is 4-quinolyl and 4-pyridyl optionally substituted with one or more radicals chosen from the definition of Y^{2A};
Y^{2A} is defined as Y²;
R^{2A} and R^{3A} taken together with the carbon atom to which they are attached form a C₃-C₁₀ cycloalkyl or heterocyclyl,
all the alkyl and phenyl above are optionally substituted with one or more radicals chosen from halogen, —OH, alk, —O-alk, —OCF₃, —S(O)ₙ—CF₃, —CF₃, —NH₂, —NH-Alk and —N(Alk)₂ and
Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹—CS—NR⁵R⁶ or —NR¹⁰—COOR¹³, all of which are optionally substituted;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
R⁷ is hydrogen, alkyl, cycloalkyl, phenyl, acyl, —S(O)₂Alk, —S(O)₂Aryl, —S(O)₂heteroaryl or —S(O)₂NR⁵R⁶,
except for Y^{1A}, B^{2A}, R^{2A} and R^{3A}, all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙR⁸, —S(O)ₙ—R⁸, —N(R¹⁰)—S(O)ₙ—NR¹¹R¹² and —S(O)ₙ—NR¹¹R¹²,
except for B^{2A}, R^{2A} and R^{3A}, all the cyclic radicals above, and also the ring formed by R⁵ and R⁶ with the nitrogen atom to which they are attached, are optionally substituted with one or more substituents selected from the group consisting of oxo and thioxo,
R⁸ is alkyl, alkenyl, cycloalkyl, cycloalkylalkyl, heterocyclyl, heterocyclylalkyl, aryl, arylalkyl, heteroaryl and heteroarylalkyl,
R⁹ is defined as R⁸ or is hydrogen,
R¹⁰ is hydrogen or alkyl,
R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl,
or R¹¹ and R¹² taken together with the nitrogen atom to which they are attached form 5- to 7-membered cyclic radical containing one or more hetero atoms chosen from O, S, N and NR, R¹³, which may be identical to or different to R⁵ or R⁶, is defined as R⁵ or R⁶, and
n is 0 to 2; or
a racemic, enantiomeric or diastereoisomeric isomer form of said compound, or addition salt with a mineral or organic acid or with mineral or organic base thereof.

3. The method of claim 2, wherein the cyclic urea compound is:

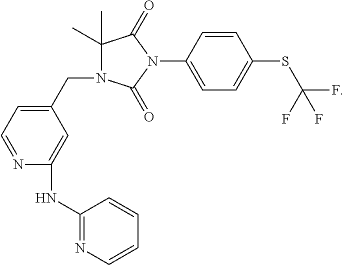 [SMILES: CC1(C)C(=O)N(c2ccc(SC(F)(F)F)cc2)C(=O)N1Cc1ccnc(Nc2ccccn2)c1]

4. The method of claim 2, wherein the cyclic urea compound is:

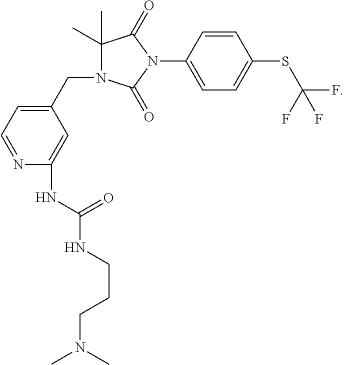 [SMILES: CN(C)CCCNC(=O)Nc1cc(CN2C(=O)N(c3ccc(SC(F)(F)F)cc3)C(=O)C2(C)C)ccn1]

5. The method of claim 1, wherein the cyclic urea compound is administered orally, intravenously, intra-peritonally, intra-articularly and/or locally to the site of desired action.

6-16. (canceled)

17. A method of treating pain in a patient in need thereof comprising administering to the patient a compound according to Formula (I): 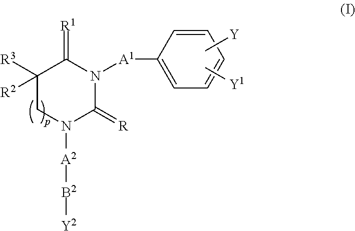
wherein:
p is 0, 1 or 2;
R and R¹, which may be identical or different, are O or NH;
R² and R³, which may be identical or different, are hydrogen, alkyl, alkenyl, alkynyl, cycloalkyl, aryl and heteroaryl that are optionally substituted, or R² and R³ taken together with the carbon atom to which they are attached form 3- to 10-membered carbocyclyl that is optionally being substituted or 3- to 10-membered heterocyclyl containing one or more hetero atoms chosen from O, S, N and NR⁷ that is optionally being substituted;
A¹ is single bond, alkyl, alkenyl or alkynyl;
Y and Y¹, which may be identical or different, are such that one of Y and Y¹ is —OCF₃, —O—F₂—CHF₂, —O—CHF₂, —O—CH₂—CF₃, —SO₂NR⁵R⁶, —SF₅ and —S(O)ₙ-alkyl and the other of Y and Y¹ is —OCF₃, —O—F₂—CHF₂, —O—CHF₂, —O—CH₂—CF₃, SO₂NR⁵R⁶, —SF₅, —S(O)ₙ-alkyl, hydrogen, halogen, hydroxyl, alkoxy, nitro, —CN, —NR⁵R⁶, optionally substituted alkyl, optionally substituted aryl, optionally substituted heteroaryl, —CF₃, —O-alkenyl, —O-alkynyl, —O-cycloalkyl, —S(O)ₙ-alkenyl, —S(O)ₙ-alkynyl, —S(O)ₙ-cycloalkyl, —CONR⁵R⁶, or free, salified or esterified carboxyl, or the 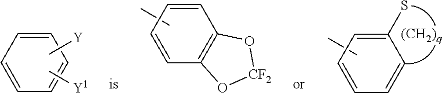
that is optionally substituted with one or more alkyl that are optionally substituted, wherein
q is 2, 3 or 4;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
A², which may be identical to or different from A¹, is defined as A¹ or is CO or SO₂,
B² is saturated or unsaturated, 3- to 10-membered monocyclic or bicyclic heterocyclyl containing one or more hetero atoms chosen from O, S, N and NR⁷ that is optionally substituted with one or more identical or different substituents defined as Y², wherein
R⁷ is hydrogen, alkyl, cycloalkyl, phenyl, acyl, —S(O)₂Alk, —S(O)₂Aryl, —S(O)₂heteroaryl or —S(O)₂NR⁵R⁶,
Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹⁰—CS—NR⁵R⁶ or —NR¹⁰—COOR¹³, all of which are optionally substituted;
all the alkyl, alkenyl, alkynyl and alkoxy above are linear or branched and contain not more than 6 carbon atoms,
all the cycloalkyl and heterocyclyl above contain not more than 7 carbon atoms,
all the aryl and heteroaryl above contain not more than 10 carbon atoms,
all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²,
all the aryl and heteroaryl above are optionally substituted with one or more substituents selected from the group consisting of alkyl, alkoxy and alkylenedioxy,
all the cyclic radicals above, and also the ring formed by R⁵ and R⁶ with the nitrogen atom to which they are attached, are optionally substituted with one or more substituents selected from the group consisting of oxo and thioxo,
n is 0 to 2, and wherein
R⁸ is alkyl, alkenyl, cycloalkyl, cycloalkylalkyl, heterocyclyl, heterocyclylalkyl, aryl, arylalkyl, heteroaryl and heteroarylalkyl,
R⁹ is defined as R⁸ or is hydrogen,
R¹⁰ is hydrogen or alkyl,
R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl, or R¹¹ and R¹² taken together with the nitrogen atom to which they are attached form 5- to 7-membered cyclic radical containing one or more hetero atoms chosen from O, S, N and NR⁷, preferably a cyclic amine, and
R¹³, which may be identical to or different to R⁵ or R⁶, is defined as R⁵ or R⁶, or a racemic, enantiomeric or diastereoisomeric isomer form of the compound of Formula (I), addition salt with mineral or organic acid or with mineral or organic base thereof, with the proviso:
a) when p is 0, R and R¹ are oxygen, A¹ is single bond or alkyl, Y and Y¹, which may be identical or different, are at least one is —OCF₃ or —S-alk, A² is single bond or alkyl and B² is an optionally substituted heterocyclyl, then R² and R³ are not one hydrogen and the other imidazolylalkyl;
b) when p is 0, R and R¹ are oxygen, A¹ is single bond or alkyl, Y and Y¹, which may be identical or different, are at least one is —OCF₃, —SO-Alk, —S(O)₂-alk or —SO₂NH₂, A² is CH₂ and B² is an optionally substituted heterocyclyl, then R² and R³ are not one hydrogen and the other alkyl optionally interrupted with O, S or N-alk; always substituted with a hydroxamate (—CO—NHOH);
c) when p is 0, R and R¹ are oxygen, A¹ is a single bond or alkyl, Y and Y¹, which may be identical or different, are at least one is —S(O)ₙ-alk, A² is single bond and B² is an optionally substituted 5- or 6-membered aromatic heterocyclyl, then R² and R³ are not selected from the group consisting of hydrogen, alkyl, arylalkyl, aryl and heteroaryl; or
d) when p is 0 to 2, R and R¹ are oxygen, A¹ is single bond, Y and Y¹, which may be identical or different, are one is —SO₂Alk or SO₂NH₂ and the other is NR⁵R⁶, A² is single bond or alkylene and B² is optionally substituted 5- to 10-membered heterocyclyl, then R² and R³ are not both hydrogen.

18. The method of claim 17, comprising administering to the patient a cyclic urea compound comprising the structure of Formula (II): 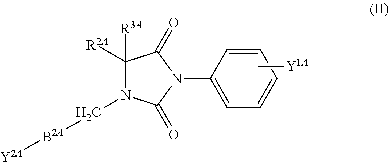
wherein:
Y^{1A} is —OCF₃, —S(O)ₙ—CF₃ and —SO₂CHF₂;
B^{2A} is 4-quinolyl and 4-pyridyl optionally substituted with one or more radicals chosen from the definition of Y^{2A};
Y^{2A} is defined as Y²;
R^{2A} and R^{3A} taken together with the carbon atom to which they are attached form a C₃-C₁₀ cycloalkyl or heterocyclyl,
all the alkyl and phenyl above are optionally substituted with one or more radicals chosen from halogen, —OH, alk, —O-alk, —OCF₃, —S(O)ₙ—CF₃, —CF₃, —NH₂, —NH-Alk and —N(Alk)₂ and
Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹—CS—NR⁵R⁶ or —NR¹⁰—COOR¹³, all of which are optionally substituted;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
R⁷ is hydrogen, alkyl, cycloalkyl, phenyl, acyl, —S(O)₂Alk, —S(O)₂Aryl, —S(O)₂heteroaryl or —S(O)₂NR⁵R⁶,
except for Y^{1A}, B^{2A}, R^{2A} and R^{3A}, all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²,
except for B^{2A}, R^{2A} and R^{3A}, all the cyclic radicals above, and also the ring formed by R⁵ and R⁶ with the nitrogen atom to which they are attached, are optionally substituted with one or more substituents selected from the group consisting of oxo and thioxo,
R⁸ is alkyl, alkenyl, cycloalkyl, cycloalkylalkyl, heterocyclyl, heterocyclylalkyl, aryl, arylalkyl, heteroaryl and heteroarylalkyl,
R⁹ is defined as R⁸ or is hydrogen,
R¹⁰ is hydrogen or alkyl,
R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl,
or R¹¹ and R¹² taken together with the nitrogen atom to which they are attached form 5- to 7-membered cyclic radical containing one or more hetero atoms chosen from O, S, N and NR⁷, R¹³, which may be identical to or different to R⁵ or R⁶, is defined as R⁵ or R⁶, and
n is 0 to 2
a racemic, enantiomeric or diastereoisomeric isomer form of said compound, or addition salt with a mineral or organic acid or with mineral or organic base thereof.

19. The method of claim 18, wherein the cyclic urea compound is:

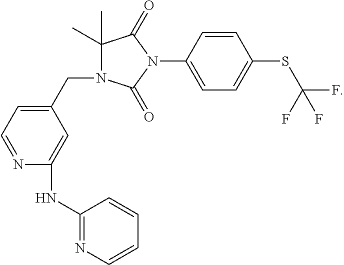 [SMILES: CC1(C)C(=O)N(c2ccc(SC(F)(F)F)cc2)C(=O)N1Cc1ccnc(Nc2ccccn2)c1]

20. The method of claim 18, wherein the cyclic urea compound is:

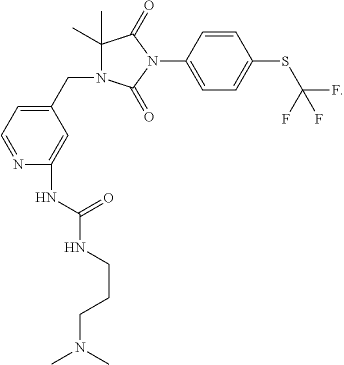 [SMILES: CN(C)CCCNC(=O)Nc1cc(CN2C(=O)N(c3ccc(SC(F)(F)F)cc3)C(=O)C2(C)C)ccn1]

21. The method of claim 17, wherein the cyclic urea compound is administered orally, intravenously, intra-peritonally, intra-articularly and/or locally to the site of desired action.

22-32. (canceled)

33. A method of inhibiting a tropomyosin-receptor kinase in a patient comprising administering to the patient a compound according to Formula (I): 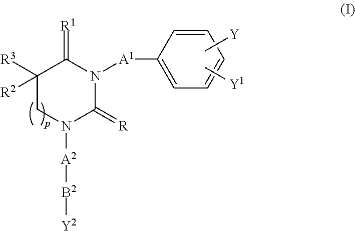
wherein:
p is 0, 1 or 2;
R and R¹, which may be identical or different, are O or NH;
R² and R³, which may be identical or different, are hydrogen, alkyl, alkenyl, alkynyl, cycloalkyl, aryl and heteroaryl that are optionally substituted, or R² and R³ taken together with the carbon atom to which they are attached form 3- to 10-membered carbocyclyl that is optionally being substituted or 3- to 10-membered heterocyclyl containing one or more hetero atoms chosen from O, S, N and NR⁷ that is optionally being substituted;
A¹ is single bond, alkyl, alkenyl or alkynyl;
Y and Y¹, which may be identical or different, are such that one of Y and Y¹ is —OCF₃, —O—F₂—CHF₂, —O—CHF₂, —O—CH₂—CF₃, —SO₂NR⁵R⁶, —SF₅ and —S(O)ₙ-alkyl and the other of Y and Y¹ is —OCF₃, —O—F₂—CHF₂, —O—CHF₂, —O—CH₂—CF₃, SO₂NR⁵R⁶, —SF₅, —S(O)ₙ-alkyl, hydrogen, halogen, hydroxyl, alkoxy, nitro, —CN, —NR⁵R⁶, optionally substituted alkyl, optionally substituted aryl, optionally substituted heteroaryl, —CF₃, —O-alkenyl, —O-alkynyl, —O-cycloalkyl, —S(O)ₙ-alkenyl, —S(O)ₙ-alkynyl, —S(O)ₙ-cycloalkyl, —CONR⁵R⁶, or free, salified or esterified carboxyl, or the 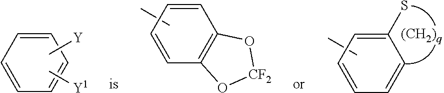
that is optionally substituted with one or more alkyl that are optionally substituted, wherein
q is 2, 3 or 4;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
A², which may be identical to or different from A¹, is defined as A¹ or is CO or SO₂,
B² is saturated or unsaturated, 3- to 10-membered monocyclic or bicyclic heterocyclyl containing one or more hetero atoms chosen from O, S, N and NR⁷ that is optionally substituted with one or more identical or different substituents defined as Y², wherein
R⁷ is hydrogen, alkyl, cycloalkyl, phenyl, acyl, —S(O)₂Alk, —S(O)₂Aryl, —S(O)₂heteroaryl or —S(O)₂NR⁵R⁶,
Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹—CS—NR⁵R⁶ or —NR¹⁰—COOR¹³, all of which are optionally substituted;
all the alkyl, alkenyl, alkynyl and alkoxy above are linear or branched and contain not more than 6 carbon atoms,
all the cycloalkyl and heterocyclyl above contain not more than 7 carbon atoms,
all the aryl and heteroaryl above contain not more than 10 carbon atoms,
all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²,
all the aryl and heteroaryl above are optionally substituted with one or more substituents selected from the group consisting of alkyl, alkoxy and alkylenedioxy,
all the cyclic radicals above, and also the ring formed by R⁵ and R⁶ with the nitrogen atom to which they are attached, are optionally substituted with one or more substituents selected from the group consisting of oxo and thioxo,
n is 0 to 2, and wherein
R⁸ is alkyl, alkenyl, cycloalkyl, cycloalkylalkyl, heterocyclyl, heterocyclylalkyl, aryl, arylalkyl, heteroaryl and heteroarylalkyl,
R⁹ is defined as R⁸ or is hydrogen,
R¹⁰ is hydrogen or alkyl,
R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl, or R¹¹ and R¹² taken together with the nitrogen atom to which they are attached form 5- to 7-membered cyclic radical containing one or more hetero atoms chosen from O, S, N and NR⁷, preferably a cyclic amine, and
R¹³, which may be identical to or different to R⁵ or R⁶, is defined as R⁵ or R⁶, or a racemic, enantiomeric or diastereoisomeric isomer form of the compound of Formula (I), addition salt with mineral or organic acid or with mineral or organic base thereof, with the proviso:
a) when p is 0, R and R¹ are oxygen, A¹ is single bond or alkyl, Y and Y¹, which may be identical or different, are at least one is —OCF₃ or —S-alk, A² is single bond or alkyl and B² is an optionally substituted heterocyclyl, then R² and R³ are not one hydrogen and the other imidazolylalkyl;
b) when p is 0, R and R¹ are oxygen, A¹ is single bond or alkyl, Y and Y¹, which may be identical or different, are at least one is —OCF₃, —SO-Alk, —S(O)₂-alk or —SO₂NH₂, A² is CH₂ and B² is an optionally substituted heterocyclyl, then R² and R³ are not one hydrogen and the other alkyl optionally interrupted with O, S or N-alk; always substituted with a hydroxamate (—CO—NHOH);
c) when p is 0, R and R¹ are oxygen, A¹ is a single bond or alkyl, Y and Y¹, which may be identical or different, are at least one is —S(O)ₙ-alk, A² is single bond and B² is an optionally substituted 5- or 6-membered aromatic heterocyclyl, then R² and R³ are not selected from the group consisting of hydrogen, alkyl, arylalkyl, aryl and heteroaryl; or
d) when p is 0 to 2, R and R¹ are oxygen, A¹ is single bond, Y and Y¹, which may be identical or different, are one is —SO₂Alk or SO₂NH₂ and the other is NR⁵R⁶, A² is single bond or alkylene and B² is optionally substituted 5- to 10-membered heterocyclyl, then R² and R³ are not both hydrogen.

34. The method of claim 33, comprising administering to the patient a cyclic urea compound comprising the structure of Formula (II): 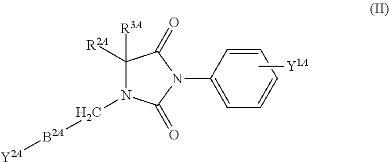
wherein:
Y^{1A} is —OCF₃, —S(O)ₙ—CF₃ and —SO₂CHF₂;
B^{2A} is 4-quinolyl and 4-pyridyl optionally substituted with one or more radicals chosen from the definition of Y^{2A};
Y^{2A} is defined as Y²;
R^{2A} and R^{3A} taken together with the carbon atom to which they are attached form a C₃-C₁₀ cycloalkyl or heterocyclyl,
all the alkyl and phenyl above are optionally substituted with one or more radicals chosen from halogen, —OH, alk, —O-alk, —OCF₃, —S(O)ₙ—CF₃, —CF₃, —NH₂, —NH-Alk and —N(Alk)₂ and
Y² is hydrogen, halogen, hydroxyl, cyano, alkyl, alkoxy, cycloalkyl, heterocyclyl, aryl, heteroaryl, —O-alkenyl, —O-alkynyl, —O-cycloalkyl, —S(O)ₙ-alkyl, —S(O)ₙ-alkenyl, —S(O)ₙ-alkynyl, —S(O)ₙ-cycloalkyl, —COOR¹³, —OCOR¹³, NR⁵R⁶, CONR⁵R⁶, —S(O)ₙ—NR⁵R⁶, —NR¹⁰—CO—R¹³, —NR¹⁰—SO₂—R¹³, NH—SO₂—NR⁵R⁶, —NR¹⁰—CO—NR⁵R⁶, —NR¹⁰—CS—NR⁵R⁶ or —NR¹⁰—COOR¹³, all of which are optionally substituted;
R⁵ and R⁶, which may be identical or different, are hydrogen, alkyl, alkenyl, alkynyl, cycloalkyl, cycloalkenyl, heterocyclyl, optionally substituted aryl or optionally substituted heteroaryl, or R⁵ and R⁶ taken together with the nitrogen atom to which they are attached form 3- to 10-membered heterocyclyl containing one or more hetero atoms chosen from O, S, N and NR⁷ that is optionally being substituted;
R⁷ is hydrogen, alkyl, cycloalkyl, phenyl, acyl, —S(O)₂Alk, —S(O)₂Aryl, —S(O)₂heteroaryl or —S(O)₂NR⁵R⁶,
except for Y^{1A}, B^{2A}, R^{2A} and R^{3A}, all the carbocyclyl, heterocyclyl, alkyl, alkenyl, alkynyl, alkoxy, cycloalkyl, heterocyclyl, aryl and heteroaryl above, and also the ring formed by R⁵ and R⁶ with the nitrogen atom to which they are attached, are optionally substituted with one or more substituents, which may be identical or different, selected from the group consisting of halogen, cyano, hydroxyl, alkoxy, —CF₃, nitro, aryl, heteroaryl, —C(═O)—OR⁹, —C(═O)—R⁸, —NR¹¹R¹², —C(═O)—NR¹¹R¹², —N(R¹⁰)—C(═O)—R⁸, —N(R¹⁰)—C(═O)—OR⁹, —N(R¹⁰)—C(═O)—NR¹¹R¹², —N(R¹⁰)—S(O)ₙ—R⁸, —S(O)ₙ—R⁸, —N(R¹⁰)—S(O)ₙ—NR¹¹R¹² and —S(O)ₙ—NR¹¹R¹²;
except for B^{2A}, R^{2A} and R^{3A}, all the cyclic radicals above, and also the ring formed by R⁵ and R⁶ with the nitrogen atom to which they are attached, are optionally substituted with one or more substituents selected from the group consisting of oxo and thioxo,
R⁸ is alkyl, alkenyl, cycloalkyl, cycloalkylalkyl, heterocyclyl, heterocyclylalkyl, aryl, arylalkyl, heteroaryl and heteroarylalkyl,
R⁹ is defined as R⁸ or is hydrogen,
R¹⁰ is hydrogen or alkyl,
R¹¹ and R¹², which may be identical or different, are hydrogen, C₃-C₆ cycloalkyl, C₁-C₄ alkyl or phenyl, optionally substituted with one or more substituents, which may be identical or different, selected from the group consisting of halogen, cyano, hydroxyl, alkoxy, —CF₃, nitro, phenyl, and free, salified, esterified or amidated carboxyl,
or R¹¹ and R¹² taken together with the nitrogen atom to which they are attached form 5- to 7-membered cyclic radical containing one or more hetero atoms chosen from O, S, N and NR⁷, R¹³, which may be identical to or different to R⁵ or R⁶, is defined as R⁵ or R⁶, and
n is 0 to 2; or
a racemic, enantiomeric or diastereoisomeric isomer form of said compound, or addition salt with a mineral or organic acid or with mineral or organic base thereof.

35. The method of claim 34, wherein the cyclic urea compound is:

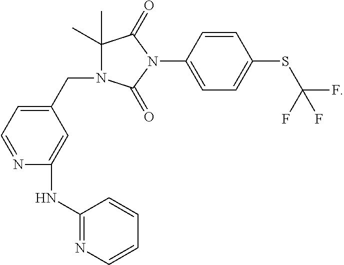 [SMILES: CC1(C)C(=O)N(c2ccc(SC(F)(F)F)cc2)C(=O)N1Cc1ccnc(Nc2ccccn2)c1]

36. The method of claim 34, wherein the cyclic urea compound is:

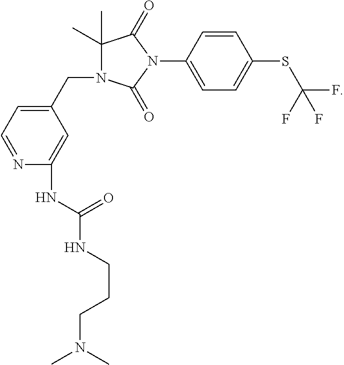 [SMILES: CN(C)CCCNC(=O)Nc1cc(CN2C(=O)N(c3ccc(SC(F)(F)F)cc3)C(=O)C2(C)C)ccn1]

37. The method of claim 33, wherein the tropomyosin-receptor kinase is tropomyosin-related kinase A.

38-40. (canceled)

41. The method of claim 33, wherein the tropomyosin-receptor kinase is tropomyosin-related kinase B.

42-44. (canceled)

45. The method of claim 33, wherein the tropomyosin-receptor kinase is tropomyosin-related kinase C.

46-48. (canceled)

49. The method of claim 33, wherein the cyclic urea compound is administered orally, intravenously, intra-peritonally, intra-articularly and/or locally to the site of desired action.

50-52. (canceled)
